(19) United States
(12) Patent Application Publication
(10) Pub. No.: US 2025/0313563 A1
(43) Pub. Date: Oct. 9, 2025

(54) GLYCOGEN SYNTHASE KINASE 3 INHIBITORS AND USES THEREOF

(71) Applicants: The Broad Institute, Inc., Cambridge, MA (US); Biogen MA Inc., Cambridge, MA (US)

(72) Inventors: Florence Fevrier Wagner, Canbridge, MA (US); Andrew George Capacci, Cambridge, MA (US); Zain Yousaf, Cambridge, MA (US); TeYu Chen, Cambridge, MA (US); Bin Ma, Cambridge, MA (US); Brian Stuart Lucas, Cambridge, MA (US); Mrinal Shekhar, Cambridge, MA (US); Kwaku Kyei-Baffour, Cambridge, MA (US); Michel Weïwer, Cambridge, MA (US); Alexandra Gould, Cambridge, MA (US)

(73) Assignees: The Broad Institute, Inc., Cambridge, MA (US); Biogen MA Inc., Cambridge, MA (US)

(21) Appl. No.: 19/175,422

(22) Filed: Apr. 10, 2025

Related U.S. Application Data
(63) Continuation of application No. PCT/US2023/076922, filed on Oct. 13, 2023.
(60) Provisional application No. 63/415,868, filed on Oct. 13, 2022.

(51) Int. Cl. C07D 471/20; A61K 31/437; A61K 31/4375; A61K 31/438; A61K 31/444; A61K 31/4545; A61K 31/4709; A61K 31/4745; A61K 31/4747; A61K 31/496; A61K 31/497; A61K 31/4985; A61K 31/501; A61K 31/5025; A61K 31/506; A61K 31/519; A61K 31/5365; A61K 31/5377; A61K 31/538; A61K 31/5383; A61K 31/55; C07D 471/04; C07D 471/10; C07D 471/14; C07D 491/20; C07D 491/22; C07D 495/20; C07D 495/22; C07D 519/00

(52) U.S. Cl. CPC C07D 471/20; A61K 31/437; A61K 31/4375; A61K 31/438; A61K 31/444; A61K 31/4545; A61K 31/4709; A61K 31/4745; A61K 31/4747; A61K 31/496; A61K 31/497; A61K 31/4985; A61K 31/501; A61K 31/5025; A61K 31/506; A61K 31/519; A61K 31/5365; A61K 31/5377; A61K 31/538; A61K 31/5383; A61K 31/55; C07D 471/04; C07D 471/10; C07D 471/14; C07D 491/20; C07D 491/22; C07D 495/20; C07D 495/22; C07D 519/00

(57) ABSTRACT

The present disclosure provides compounds of Formulae I-A, I-B, II-A, II-B, III-A, III-B, IV-A, IV-B, V-A, V-B, VI-A, and VI-B, and compounds shown in Table 13, Table 13A, and Table 14, and pharmaceutically acceptable salts, solvates, hydrates, polymorphs, co-crystals, tautomers, stereoisomers, isotopically labeled compounds, and prodrugs thereof, which are GSK3 inhibitors. The present disclosure also provides pharmaceutical compositions, combination therapies, and kits comprising the compounds, and pharmaceutically acceptable salts, solvates, hydrates, polymorphs, co-crystals, tautomers, stereoisomers, isotopically labeled compounds, or prodrugs thereof, and methods of treating or preventing diseases and disorders associated with GSK3.

### RELATED APPLICATIONS

The present application claims priority under 35 U.S.C. § 119 (e) to U.S. Provisional Patent Application No. 63/415,868, filed Oct. 13, 2022, which is incorporated herein by reference.

### BACKGROUND

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-I) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, metabolic disorders (e.g., diabetes), and hormone-related diseases. Accordingly, there remains a need to find protein kinase inhibitors, particularly glycogen synthase kinase 3 (GSK3) inhibitors, useful as therapeutic agents. GSK3 inhibitors have been reported in, e.g., U.S. patent application numbers US-2014-0107141-A1 and US-2016-0375006-A1, each of which is incorporated herein by reference in its entirety.

### SUMMARY OF THE DISCLOSURE

The present disclosure relates in part to compounds (e.g., compounds of Formulae I-A, I-B, II-A, II-B, III-A, III-B, IV-A, IV-B, V-A, V-B, VI-A, and VI-B, and shown in Table 13, Table 13A, and Table 14, and pharmaceutically acceptable salts, solvates, hydrates, polymorphs, co-crystals, tautomers, stereoisomers, isotopically labeled compounds, and prodrugs thereof (collectively, “compounds provided herein”)). The compounds provided herein may inhibit GSK3. The compounds provided herein may selectively inhibit GSK3 (e.g., selectively inhibit GSK3α over GSK3β or selectively inhibit GSK3β over GSK3α). The compounds provided herein may be advantageous over known GSK3 inhibitors (e.g., non-selective GSK3 inhibitors) at least in part because the former may reduce or eliminate off-target effects. The present disclosure also provides pharmaceutical compositions and kits comprising the compounds provided herein. The present disclosure also provides methods of treating or preventing a disease, as well as methods of inhibiting the activity and/or production of a GSK3.

In one aspect, the present disclosure provides compounds of Formula I-A or I-B:

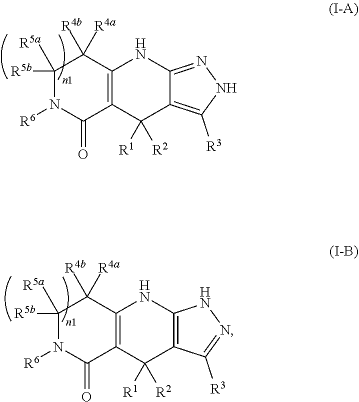

and pharmaceutically acceptable salts, solvates, hydrates, polymorphs, co-crystals, tautomers, stereoisomers, isotopically labeled compounds, and prodrugs thereof, wherein the moieties and variables included in Formula I-A and I-B are as defined herein.

In another aspect, the present disclosure provides compounds of Formula II-A or II-B:

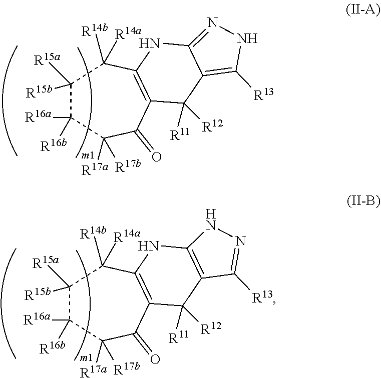

and pharmaceutically acceptable salts, solvates, hydrates, polymorphs, co-crystals, tautomers, stereoisomers, isotopically labeled compounds, and prodrugs thereof, wherein the moieties and variables included in Formula II-A or II-B are as defined herein.

In another aspect, the present disclosure provides compounds of Formula III-A or III-B:

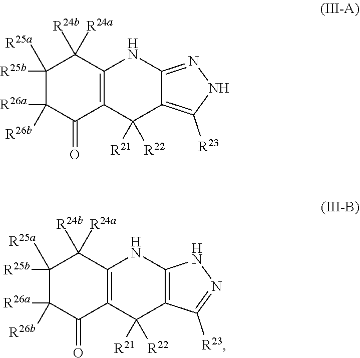

and pharmaceutically acceptable salts, solvates, hydrates, polymorphs, co-crystals, tautomers, stereoisomers, isotopically labeled compounds, and prodrugs thereof, wherein the moieties and variables included in Formula III-A or III-B are as defined herein.

In another aspect, the present disclosure provides compounds of Formula IV-A or IV-B:

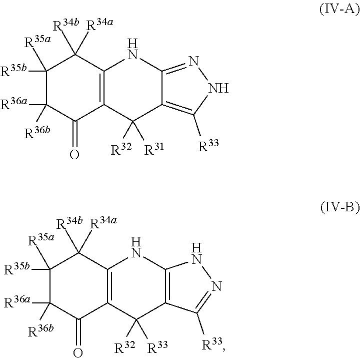

and pharmaceutically acceptable salts, solvates, hydrates, polymorphs, co-crystals, tautomers, stereoisomers, isotopically labeled compounds, and prodrugs thereof, wherein the moieties and variables included in Formula IV-A or IV-B are as defined herein.

In another aspect, the present disclosure provides compounds of Formula V-A or V-B:

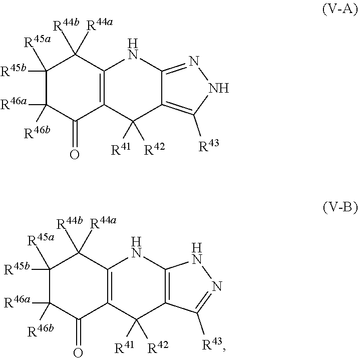

and pharmaceutically acceptable salts, solvates, hydrates, polymorphs, co-crystals, tautomers, stereoisomers, isotopically labeled compounds, and prodrugs thereof, wherein the moieties and variables included in Formula V-A or V-B are as defined herein.

In another aspect, the present disclosure provides compounds of Formula VI-A or VI-B:

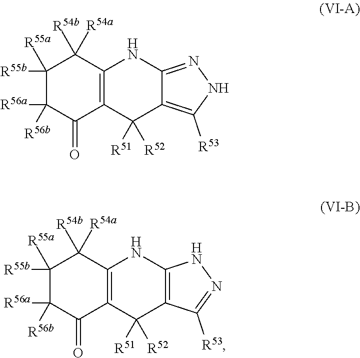

and pharmaceutically acceptable salts, solvates, hydrates, polymorphs, co-crystals, tautomers, stereoisomers, isotopically labeled compounds, and prodrugs thereof, wherein the moieties and variables included in Formula VI-A or VI-B are as defined herein.

In another aspect, the present disclosure provides pharmaceutical compositions comprising a compound provided herein and optionally a pharmaceutically acceptable excipient.

In another aspect, the present disclosure provides kits comprising a compound provided herein or pharmaceutical composition provided herein and instructions for using the compound or pharmaceutical composition.

In another aspect, the present disclosure provides methods for treating diseases in a subject in need thereof, the methods comprising administering to the subject an effective amount of a compound provided herein or a compound of the formula:

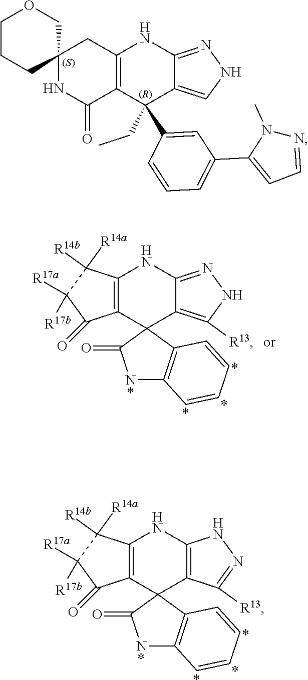

or a pharmaceutically acceptable salt, solvate, hydrate, polymorph, co-crystal, tautomer, stereoisomer, isotopically labeled compound, or prodrug thereof, wherein the moieties and variables are as defined herein.

In another aspect, the present disclosure provides methods for preventing diseases in a subject in need thereof, the methods comprising administering to the subject an effective amount of a compound provided herein or a compound of the formula:

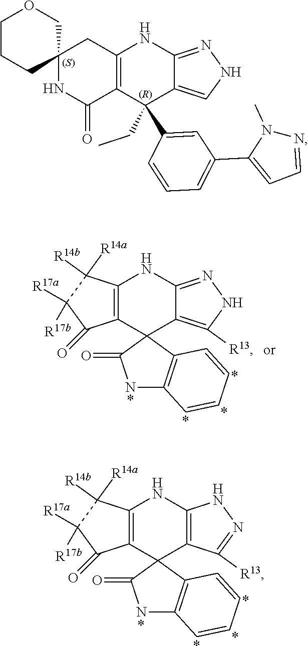

or a pharmaceutically acceptable salt, solvate, hydrate, polymorph, co-crystal, tautomer, stereoisomer, isotopically labeled compound, or prodrug thereof, wherein the moieties and variables are as defined herein.

In another aspect, the present disclosure provides methods of inhibiting the activity and/or production of a glycogen synthase kinase 3 (GSK3) in a subject in need thereof, the method comprising administering to the subject an effective amount of a compound provided herein or a compound of the formula:

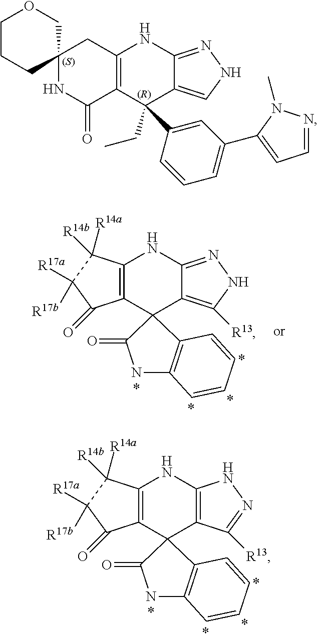

or a pharmaceutically acceptable salt, solvate, hydrate, polymorph, co-crystal, tautomer, stereoisomer, isotopically labeled compound, or prodrug thereof, wherein the moieties and variables are as defined herein.

In another aspect, the present disclosure provides methods of inhibiting the activity and/or production of a glycogen synthase kinase 3 (GSK3) in a cell, tissue, or biological sample the method comprising contacting the cell, tissue, or biological sample with an effective amount of a compound provided herein or a compound of the formula:

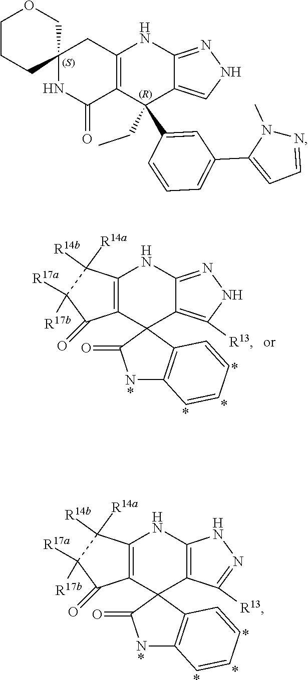

or a pharmaceutically acceptable salt, solvate, hydrate, polymorph, co-crystal, tautomer, stereoisomer, isotopically labeled compound, or prodrug thereof, wherein the moieties and variables are as defined herein.

The details of one or more embodiments of the disclosure are set forth herein. Other features, objects, and advantages of the disclosure will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. In some embodiments, isomers are isolated from mixtures by methods known to those skilled in the art, including chiral high pressure liquid chromatography (HPLC) and the formation and crystallization of chiral salts; or preferred isomers are prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 5 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include-CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to carbon atoms (“C₁₋₆ alkenyl”). In some embodiments, an alkenyl group has 1 to 5 carbon atoms (“C₁₋₅ alkenyll”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). In some embodiments, the one or more carbon-carbon double bonds is internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In some embodiments, in an alkenyl group, a C═C double bond for which the stereochemistry is not specified (e.g., —CH═CHCH₃ or

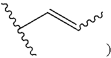

is in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₂ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₆ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). In some embodiments, the one or more carbon-carbon triple bonds is internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to carbon atoms, at least one triple bond, and 1 or 2 heteroatoms within the parent chain (“heteroC₁₋alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to ring carbon atoms (“C₃₋₁₀ carbocyclyl”). In some embodiments, a carbocyclyl group has 3 to 8 ring carbon atoms (“C₃₋₂ carbocyclyl”). In some embodiments, a carbocyclyl group has 3 to 7 ring carbon atoms (“C₃₋₇ carbocyclyl”). In some embodiments, a carbocyclyl group has 3 to ring carbon atoms (“C₃₋₆ carbocyclyl”). In some embodiments, a carbocyclyl group has 4 to ring carbon atoms (“C₄₋₆ carbocyclyl”). In some embodiments, a carbocyclyl group has 5 to ring carbon atoms (“C₅₋₆ carbocyclyl”). In some embodiments, a carbocyclyl group has 5 to ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₋₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and is saturated or contains one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In some embodiments, in heterocyclyl groups that contain one or more nitrogen atoms, the point of attachment is a carbon or nitrogen atom, as valency permits. In some embodiments, a heterocyclyl group is either monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and is either saturated or contains one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having 26 ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₋₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In some embodiments, in heteroaryl groups that contain one or more nitrogen atoms, the point of attachment is a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. In some embodiments, in polycyclic heteroaryl groups wherein one ring does not contain a heteroatom (e.g., indolyl, quinolinyl, carbazolyl, and the like) the point of attachment is on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

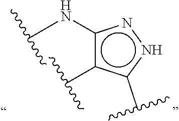

refers to

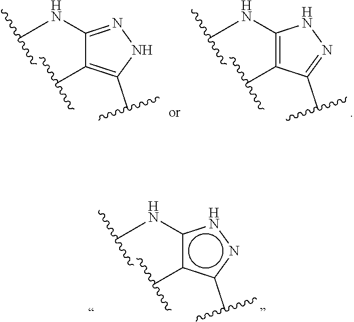

refers to

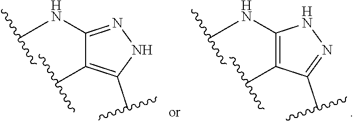 [SMILES: *Nc1n[nH]c(*)c1* ; *Nc1[nH]nc(*)c1*]

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, =NNR^{bb}C(═O)R^{aa}, =NNR^{bb}C(═O)OR^{aa}, =NNR^{bb}S(═O)₂R^{aa}, =NR^{bb}, or =NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc}), —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SO₂R^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R⁶ groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, CN, —NO₂, —N₃, —SO₂H, —SO₃H, —OH, —OR^{ee}, —ON(R^{ff})₂, N(R^{ff})₂, N(R_{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(O)R^{ee}, —Si(R^{ee})₃, —OSi(Rₑₑ)₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ee} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, N(C₁₋₆ alkyl)₃⁺X⁻, —NH(C₁₋₆ alkyl)₂⁺X⁻, —NH₂(C₁₋₆ alkyl) ⁺X⁻, —NH₃⁺X⁻, —N(OC₁₋₆ alkyl)(C₁₋₆ alkyl), —N(OH) (C₁₋₆ alkyl), —NH(OH), —SH, —SC₁₋₆ alkyl, —SS(C₁₋₆ alkyl), —C(═O)(C₁₋₆ alkyl), —CO₂H, —CO₂ (C₁₋₆ alkyl), —OC(═O)(C₁₋₆ alkyl), —OCO₂(C₁₋₆ alkyl), —C(═O)NH₂, —C(═O) N(C₁₋₆ alkyl)₂, OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S) N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents are joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “halo” or “halogen” refers to fluorine (fluoro, —F), chlorine (chloro, —C₁), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S)N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{bb})₂, and —NHP(═O)(N(R^{bb})₂)₂, wherein R^{aa}, R^{bb} and R^{cc} are as defined herein, and wherein Rob of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}COR^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)Rₐₐ, —C(═O)OR^{aa}, —C(═O)—O—C(═O)R^{aa}, —C(═O)SR^{aa}, —C(═O)N(R^{bb})₂, —C(═S)R^{aa}, —C(═S) N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. In some embodiments, the term “acyl” refers to a group having the general formula —C(═O)R^{aa}, —C(═O)OR^{aa}, —C(═O)—O—C(═O)R^{aa}, —C(═O)SR^{aa}, or —C(═O)N(R^{bb})₂.

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O) NR^{bb}SOR^{aa}, —C(═S)N(R^{bb})₂), and imines (—C(═NR^{bb})R^{aa}, —C(═NR^{bb})O)R^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein. Nitrogen atoms are substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —COR^{aa}, —SO₂R^{aa}, —C(═NR^{bb})Rₐₐ, —C(═NR^{cc})OR^{aa}, —C(═NR^{cc})N(R^{cc})₂, —SO₂N(R^{cc})₂, —SO₂R^{cc}₂, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitrophenylacetamide, o-nitrophenoxyacetamide, acetoacetamide, (N′-dithiobenzyloxyacylamino) acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo) fluorenylmethyl carbamate, 9-(2,7-dibromo) fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, f-butyl carbamate (BOC or Boc), 1-adamantyl carbamate (Adoc), vinyl carbamate (Voc), allyl carbamate (Alloc), I-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-f-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxy benzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl) ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N—[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N-(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SOR^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p, p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri (p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3′″-[N-(imidazolylethyl) carbamoyl]trityl Ether (IETr-OR), 1,1-bis(4-methoxyphenyl)-1′-pyrenylmethyl, 9-anthryl, 9-(9-phenyl) xanthenyl, 9-(9-phenyl-10-oxo) anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), f-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl) phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′ N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc}, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors. A “counterion” or “anionic counterion” is a negatively charged group associated with a positively charged group in order to maintain electronic neutrality. In some embodiments, an anionic counterion is monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinato, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THE, diethyl ether, and the like. In some embodiments, the compounds provided herein are prepared, e.g., in crystalline form. In some embodiments, the compounds provided herein are prepared, e.g., in crystalline form, and are solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound, or a salt thereof, that is associated with water. Typically, the number of the water molecules contained in a hydrate of a compound, or a salt thereof, is in a definite ratio to the number of the compound molecules in the hydrate. Therefore, in some embodiments, a hydrate of a compound, or a salt thereof, is represented, for example, by the general formula R·x H₂O, wherein R is the compound, or a salt thereof, and x is a number greater than 0. A given compound, or a salt thereof, may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base.

Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. In some embodiments, an enantiomer is characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. In some embodiments, co-crystals are useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility.

Recrystallization solvent, rate of crystallization, storage temperature, and other factors may cause one crystal form to dominate. In some embodiments, various polymorphs of a compound (or a salt, hydrate, or solvate thereof) are prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. In some embodiments, C₁-C₈alkyl, C₂-C₅ alkenyl, C₂-C₅ alkynyl, aryl, C₇-C₁₂ substituted aryl, and C₇-C₁₂ arylalkyl esters of the compounds described herein are preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. In some embodiments, the non-human animal is a male or female at any stage of development. In some embodiments, the non-human animal is a transgenic animal or genetically engineered animal. The term “patient” refers to a human subject in need of treatment of a disease.

The term “tissue” refers to any biological tissue of a subject (including a group of cells, a body part, or an organ) or a part thereof, including blood and/or lymph vessels. In some embodiments, “tissue” is the object to which a compound, particle, and/or composition of the disclosure is delivered. In some embodiments, a tissue is an abnormal or unhealthy tissue, which may need to be treated. A tissue may also be a normal or healthy tissue that is under a higher than normal risk of becoming abnormal or unhealthy, which may need to be prevented.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “kinase” represents transferase class enzymes that are able to transfer a phosphate group from a donor molecule to an acceptor molecule, e.g., an amino acid residue of a protein or a lipid molecule. Representative, non-limiting examples of kinases include Abl, ACK, Akt1/PKBα, Akt2/PKBβ, Akt3/PKBγ, ALK1, ALK2, Alk4, AMPKα1/β1/γ1, AMPKα1/β1/γ2, AMPKα1/β1/γ3, AMPKα1/β2/γ1, AMPKα2/β1/γ1, AMPKα2/β2/γ2, Abl2, ARKS, Ask1, Aurora A, Aurora B, Aurora C, Axl, BARK1, Blk, Bmx, B-Raf, Brk, BrSK1, BrSK2, Btk, CaMK1α, CaMK1β, CaMK1γ, CaMK1δ, CAMK2α, CaMK2β, CAMK2δ, CAMK2γ, CAMK4, CAMKK1, CAMKK2, CDK1, CDK2, CDK3, CDK4, CDK5, CDK6, CDK7, CDK9, CDK1/cyclin B, CDK2/cyclin A, CDK2/cyclin E, CDK3/cyclin E, CDK5/p25, CDK5/p35, CDK6/cyclinD3, CDK7/cyclin H/MAT1, CDK9/cyclin T1, CHK1, CHK2, CK1α, CK1γ, CK1δ, CK1ε, CK1β1, CK1γ1, CK1γ2, CK1γ3, CK2α1, CK2α2, cKit, c-RAF, CLK1, CLK2, CLK3, COT, Csk, DAPK1, DAPK2, DAPK3, DCAMLK2, DDR2, DMPK, DRAK1, DYRK1A, DYRK2, DYRK3, eEF2K, EGFR, EPHA1, EPHA2, EPHA3, EPHA4, EPHA5, EPHA6, EPHA7, EPHA8, EphB1, EphB2, EphB3, EphB4, ErbB4, Erk1, Erk2, FAK, Fer, Fes, FGFR1, Flt2, Flt4, FLT3 D835Y, FGFR2, FGFR3, FGFR4, Fgr, Flt1, Flt3, Fms, FRK, FynA, GCK, GPRK5, GRK2, GRK4, GRK6, GRK7, GSK3α, GSK3β, Hck, HER2, HER4, HIPK1, HIPK2, HIPK3, HIPK4, IGF1R, IKKβ, IKKα, IKKε, IR, InsR, IRR, IRAK1, IRAK2, IRAK4, Itk, JAK2, JAK3, JNK1, JNK2, JNK3, KDR, KHS1, Kit, Lck, LIMK1, LKB1, LOK, LRRK2, Lyn A, Lyn B, MAPK1, MAPK2, MAPK12, MAPKAP-K2, MAPKAP-K3, MAPKAPK2, MAPKAPK3, MAPKAPK5, MARK1, MARK2, MARK3, MARK4, MELK, MEK1, MEK2, MEKK2, MEKK3, Mer, Met, MET M1250T, MINK, MKK4, MKK6, MKK7β, MLCK, MLK1, MLK3, MNK1, MNK2, MRCKα, MRCKβ, MSK1, MSK2, MSSK1, STK23, STK4, STK3, STK24, MST1, MST2, MST3, MST4, MUSK, mTOR, MYO3β, MYT1, NDR1, NEK11, NEK2, NEK3, NEK6, NEK7, NEK9, NLK, NUAK2, p38α, p38β, p38δ, p38γ, p70S6K, S6K, SRK, PAK1/CDC42, PAK2, PAK3, PAK4, PAK5, PAK6, PAR-1Bα, PASK, PBK, PDGFRα, PDGFRβ, PDK1, PEK, PHKG2, PI3Kα, PI3Kβ, PI3Kγ, PI3Kδ, Pim1, Pim2, PKAcα, PKAcβ, PKAcγ, PKA (b), PKA, PKBα, PKBβ, PKBγ, PKCα, PKCβ1, PKCβ2, PKCβ11, PKCδ, PKCε, PKCγ, PKCμ, PKCη, PKC₁, PKCθ, PKCζ, PKD1, PKD2, PKD3, PKG1α, PKG1β, PKN1, PKN2, PKR, PLK1, PLK2, PLK3, PLK4, Polo, PRAK, PRK2, PrKX, PTK5, PYK2, QIK, Raf1, Ret, RIPK2, RIPK5, ROCK1, ROCK2, RON, ROS, Rse, RSK1, RSK2, RSK3, RSK4, SAPK2a, SAPK2b, SAPK3, SAPK4, SGK1, SGK2, SGK3, SIK, MLCK, SLK, Snk, Src, SRDK1, SRDK2, STK33, SYK, TAK1-TAB1, TAK1, TBK1, TAO1, TAO2, TAO3, TBK1, TEC, TESK1, TGFβR1, TGFβR2, Tie2, TLK2, TrkA, TrkB, TrkC, TSSK1, TSSK2, TTK, TXK, TYK2, TYRO3, ULK1, ULK2, WEE1, WNK2, WNK3, Yes1, YSK1, ZAK, ZAP70, ZC3, and ZIPK.

The term “administer,” “administering,” or “administration” refers to implanting, absorbing, ingesting, injecting, inhaling, or otherwise introducing a compound provided herein, a compound useful in a provided method, or a pharmaceutical composition provided herein, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment is administered after one or more signs or symptoms of the disease have developed or have been observed. In other embodiments, treatment is administered in the absence of signs or symptoms of the disease. For example, in some embodiments, treatment is administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound described herein refers to an amount sufficient to elicit the desired biological response. An effective amount of a compound described herein May vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting the activity and/or production of a GSK3. In certain embodiments, a therapeutically effective amount is an amount sufficient for treating a disease. In certain embodiments, a therapeutically effective amount is an amount sufficient for inhibiting the activity and/or production of a GSK3 and treating a disease.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting the activity and/or production of a GSK3. In certain embodiments, a prophylactically effective amount is an amount sufficient for preventing a disease. In certain embodiments, a prophylactically effective amount is an amount sufficient for inhibiting the activity and/or production of a GSK3 and preventing a disease.

The term “inhibit” or “inhibition”, for example, in the context of a GSK3, refers to a reduction in activity or production. In some embodiments, the term refers to a reduction of the level of activity and/or production, e.g., GSK3 activity and/or production, to a level that is statistically significantly lower than an initial level, which may, for example, be a baseline level of activity and/or production. In some embodiments, the term refers to a reduction of the level of activity and/or production, e.g., GSK3 activity and/or production, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of activity and/or production.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include Aarskog-Scott syndrome, Aase syndrome, achondroplasia, acrodysostosis, addiction, adrenoleukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher's disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25^{th} ed.; Hensyl ed.; Williams & Wilkins: Philadelphia, 1990. The cancer may be a solid tumor. The cancer may be a hematological malignancy. Exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, dermatitis (e.g., stasis dermatitis, allergic contact dermatitis, atopic dermatitis, irritant contact dermatitis, neurodermatitis perioral dermatitis, seborrheic dermatitis), hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, necrotizing enterocolitis, inflammatory rosacea. An ocular inflammatory disease includes post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barre syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenia purpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including Alzheimer's disease, Parkinson's disease, amyotrophic lateral sclerosis, tauopathies (including frontotemporal dementia), and Huntington's disease. Examples of neurological diseases include headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizacus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawl symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “metabolic disease” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include the endocrine (hormonal) control system (e.g., the insulin pathway, the enteroendocrine hormones including GLP-1, PYY or the like), the neural control system (e.g., GLP-1 in the brain), or the like. Examples of metabolic disorders include diabetes (e.g., Type I diabetes, Type II diabetes, gestational diabetes), hyperglycemia, hyperinsulinemia, insulin resistance, and obesity.

The term “psychiatric disorder” refers to a condition or disorder relating to the functioning of the brain and the cognitive processes or behavior. Psychiatric disorders may be further classified based on the type of neurological disturbance affecting the mental faculties. Psychiatric disorders are expressed primarily in abnormalities of thought, feeling, emotion, and/or behavior producing either distress or impairment of function (for example, impairment of mental function such with dementia or senility). The term “psychiatric disorder” is, accordingly, sometimes used interchangeably with the term “mental disorder” or the term “mental illness”.

A psychiatric disorder is often characterized by a psychological or behavioral pattern that occurs in an individual and is thought to cause distress or disability that is not expected as part of normal development or culture. Definitions, assessments, and classifications of mental disorders can vary, but guideline criteria listed in the International Classification of Diseases and Related Health Problems (ICD, published by the World Health Organization, WHO), or the Diagnostic and Statistical Manual of Mental Disorders (DSM, published by the American Psychiatric Association, APA) and other manuals are widely accepted by mental health professionals. Individuals may be evaluated for various psychiatric disorders using criteria set forth in these and other publications accepted by medical practitioners in the field and the manifestation and severity of a psychiatric disorder may be determined in an individual using these publications.

Categories of diagnoses in these schemes may include dissociative disorders, mood disorders, anxiety disorders, psychotic disorders, eating disorders, developmental disorders, personality disorders, and other categories. There are different categories of mental disorder, and many different facets of human behavior and personality that can become disordered.

One group of psychiatric disorders includes disorders of thinking and cognition, such as schizophrenia and delirium. A second group of psychiatric disorders includes disorders of mood, such as affective disorders and anxiety. A third group of psychiatric disorders includes disorders of social behavior, such as character defects and personality disorders. And a fourth group of psychiatric disorders includes disorders of learning, memory, and intelligence, such as mental retardation and dementia. Accordingly, psychiatric disorders encompass schizophrenia, delirium, attention deficit disorder (ADD), schizoaffective disorder, depression (e.g., lithium-resistant depression), mania, attention deficit disorders, drug addiction, dementia, agitation, apathy, anxiety, psychoses, personality disorders, bipolar disorders, unipolar affective disorder, obsessive-compulsive disorders, eating disorders, post-traumatic stress disorders, irritability, adolescent conduct disorder and disinhibition.

Some diseases classified as neurodegenerative diseases, for example Alzheimer's disease, also sometimes show aspects of psychiatric disorders as listed herein, for example disorders of memory or dementia. Some neurodegenerative diseases or manifestations thereof can, accordingly, also be referred to as psychiatric disorders. These terms are, therefore, not mutually exclusive.

The state of anxiety or fear can become disordered, so that it is unusually intense or generalized over a prolonged period of time. Commonly recognized categories of anxiety disorders include specific phobia, generalized anxiety disorder, social anxiety disorder, panic disorder, agoraphobia, obsessive-compulsive disorder, post-traumatic stress disorder.

Relatively long lasting affective states can also become disordered. Mood disorder involving unusually intense and sustained sadness, melancholia or despair is known as clinical depression (or major depression), and may more generally be described as emotional dysregulation. Milder but prolonged depression can be diagnosed as dysthymia. Bipolar disorder involves abnormally “high” or pressured mood states, known as mania or hypomania, alternating with normal or depressed mood.

Patterns of belief, language use and perception can become disordered. Psychotic disorders centrally involving this domain include schizophrenia and delusional disorder. schizoaffective disorder is a category used for individuals showing aspects of both schizophrenia and affective disorders. Schizotypy is a category used for individuals showing some of the traits associated with schizophrenia but without meeting cut-off criteria.

The fundamental characteristics of a person that influence his or her cognitions, motivations, and behaviors across situations and time—can be seen as disordered due to being abnormally rigid and maladaptive. Categorical schemes list a number of different personality disorders, such as those classed as eccentric (e.g., paranoid personality disorder, schizoid personality disorder, schizotypal personality disorder), those described as dramatic or emotional (antisocial personality disorder, Borderline personality disorder, histrionic personality disorder, narcissistic personality disorder) or those seen as fear-related (avoidant personality disorder, dependent personality disorder, obsessive-compulsive personality disorder).

### BRIEF DESCRIPTION OF THE DRAWINGS

FIGS. 1A to ID show that both GSK3α and GSKβ isoforms phosphorylated tau at multiple epitopes including the disease enriched epitopes Thr231 and S202/Thr205 (FIG. 1A). The percent inhibition of pTh231 was then compared using AZ1080 (FIG. 1B), BRD0705 (BRD-0705; FIG. 1C), and 837646 (FIG. 1D).

FIGS. 2A to 2B show binding of GSK3 small molecule inhibitors as a function of concentration at ATP binding pocket and had similar residence times. Inhibitor compounds bound competitively with the kinase tracer (FIG. 2A). Inhibitors had similar residence times for GSK isoforms (FIG. 2B).

FIGS. 3A to 3C show the combined inhibition of both GSK3 paralogs may be necessary for nuclear translocation of β-catenin in SH-SYSY cells. FIGS. 3A to 3C show IC₅₀ curves demonstrating potency of GSK3 inhibitors to cause β-catenin translocation to the nucleus, exemplary pictures at the 20 μM, and IC₅₀ data for β-catenin, Thr231 assay for GSKα, and Thr231 assay for GSKβ for compound BRD-0705 (FIG. 3A), 837646 (FIG. 3B), and AZ 1080 (FIG. 3C).

FIGS. 4A to 4D show selective GSK3α inhibition by 837646 reduced tau phosphorylation at disease relevant sites in vivo. FIG. 4A shows T231 normalized to total tau versus time post dose. FIG. 4B shows total concentration over time. FIG. 4C shows percent engagement of GSK3α over time. FIG. 4D shows percent engagement of GSK3ß over time.

FIG. 5 shows the elucidation of a GSK3α crystal structure.

FIGS. 6A to 6D show Thr231 cell-based assay results comparing percent inhibition of pThr231, percent to untreated mBU, and BRET ratios in 947651 and 948546. FIG. 6A shows potency of 947651 and 948546 as determined by Thr231 cell-based assay. FIG. 6B shows potency of 947651 and 948546 as determined by nanobret assay. FIG. 6C shows residence times of 947651 and 948546 for GSK3α. FIG. 6D shows potency of 947651 and 948546 to induce translocation of β-catenin to the nucleus.

FIGS. 7A to 7B depict 948546 bound GSK3α versus GSKβ (FIG. 7A), as well as α/β pocket surface overlay (FIG. 7B).

FIG. 8 shows the GSK3 isoform phosphorylation of disease-relevant epitopes of tau.

FIG. 9 shows the specificity of Thr231 using a plate based assay.

FIGS. 10A to 10B show activity of GSK3 inhibitors BRD0705 (FIG. 10A) and 837646 (FIG. 10B) on tau phosphorylation at Ser202 and Thr205 as a function of log concentration.

FIG. 11 shows the EC₅₀ for Tracer 8 at GSK3α (“GSK-A”; FIG. 11A) and GSK3β (“GSK-B”; FIG. 11B) in HEK293T cells transiently transfected with either NanoLuc-GSK3α or NanoLuc-GSK3β plasmids, treated for two hours with NanoBRET Tracer-8 the following day, and incubated in the presence or absence of a saturating dose of GSK3 inhibitor compound 837646 at 10 μM.

FIG. 12 shows the PK properties for PPB Fu % (rat) 837646.

FIG. 13 depicts free drug concentration based on plasma protein binding for 837646.

FIG. 14 shows the activation of β-catenin following treatment with 947651 or 948546.

### DETAILED DESCRIPTION OF CERTAIN EMBODIMENTS

The present disclosure provides compounds (e.g., compounds of Formulae I-A, I-B, II-A, II-B, III-A, III-B, IV-A, IV-B, V-A, V-B, VI-A, and VI-B, and shown in Table 13, Table 13A, and Table 14, pharmaceutically acceptable salts, solvates, hydrates, polymorphs, co-crystals, tautomers, stereoisomers, isotopically labeled compounds, and prodrugs thereof), and pharmaceutical compositions and kits thereof. Also provided herein are methods of treating and/or preventing a disease in a subject in need thereof, as well as methods of inhibiting the activity and/or production GSK3 in a subject in need thereof, cell, or tissue in vivo or in vitro.

The contents of International PCT Application Publication Number WO 2018/187630 are incorporated herein by reference in their entirety.

### Compounds

In one aspect, provided herein is a compound, or a pharmaceutically acceptable salt, solvate, hydrate, polymorph, co-crystal, tautomer, stereoisomer, isotopically labeled compound, or prodrug thereof, wherein the compound is of Formula I-A or I-B:

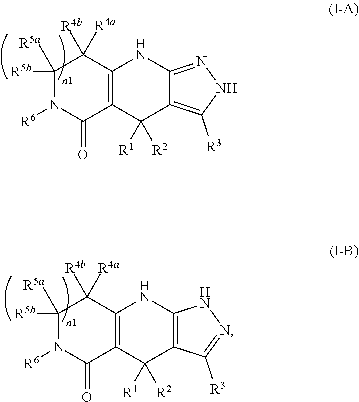

wherein:
[symbol] is a single or double bond;
R¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R² is optionally substituted alkyl, optionally substituted alkenyl, or optionally substituted alkynyl;
or R¹ and R² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
R³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)N(R^{A}), —OC(═O)R^{A}, —OC(═O)OR^{A}, —OC(═O)SR^{A}, —OC(═O)N(R^{A})₂, —OC(═NR^{A})R^{A}, —OC(NR^{A})OR^{A}, —OC(═NR^{A})SR^{A}, —OC(═NR^{A})N(R^{A})₂, —OS(O)R^{A}, —OS(═O)OR^{A}, —OS(═O)SR^{A}, —OS(═O)N(R^{A})₂, —OS(═O)R^{A}, —OS(O)OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, or —OSi(OR^{A})₃;
each of R^{4a} and R^{4b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each instance of R^{A} is independently hydrogen, optionally substituted alkyl, optionally substituted alkenyl, optionally substituted alkynyl, optionally substituted heteroalkyl, optionally carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring; and
or R^{4a} and R^{4b} are taken together with their intervening atom to form an optionally substituted, monocyclic, carbocyclic or heterocyclic ring;
R⁶ is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl; and
n1 is 0 or 1;
when n1 is 1:
   each of R^{5a} and R^{5b} independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or R^{5a} and R^{5b} are taken together with their intervening atom to form an optionally substituted, monocyclic, carbocyclic or heterocyclic ring;
   or R^{4b} and R^{5a} are taken together with their intervening atom to form an optionally substituted, monocyclic, carbocyclic, heterocyclic, aryl, or heteroaryl ring;
provided that the compound is not of the formula:

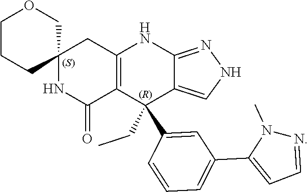

In some embodiments, the compound is of the formula:

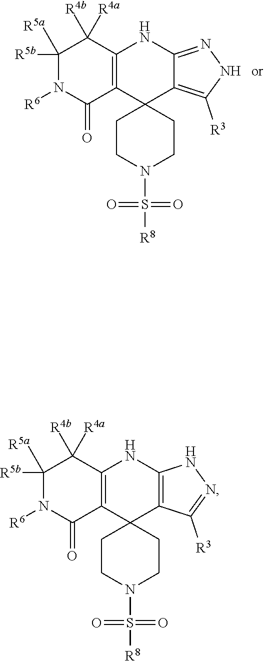

or a pharmaceutically acceptable salt, solvate, hydrate, polymorph, co-crystal, tautomer, stereoisomer, isotopically labeled compound, or prodrug thereof.

In some embodiments, the compound is of the formula:

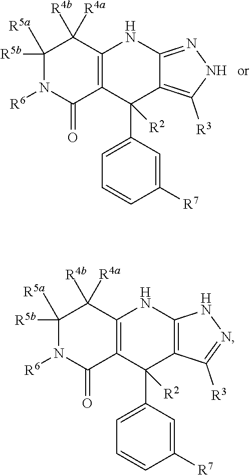

or a pharmaceutically acceptable salt, solvate, hydrate, polymorph, co-crystal, tautomer, stereoisomer, isotopically labeled compound, or prodrug thereof.

In some embodiments, the compound is of the formula:

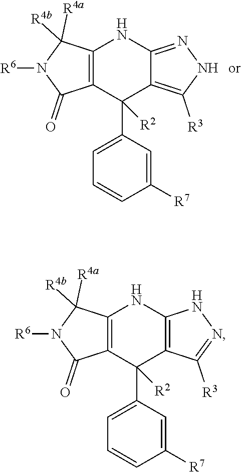

or a pharmaceutically acceptable salt, solvate, hydrate, polymorph, co-crystal, tautomer, stereoisomer, isotopically labeled compound, or prodrug thereof.

In some embodiments, the compound is of the formula:

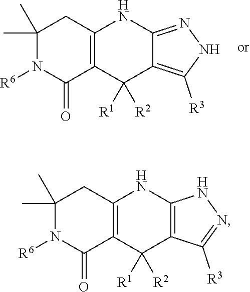

or a pharmaceutically acceptable salt, solvate, hydrate, polymorph, co-crystal, tautomer, stereoisomer, isotopically labeled compound, or prodrug thereof.

In some embodiments, [symbol] is a single bond. In some embodiments, [symbol] is a double bond.

In some embodiments, n1 is 0. In some embodiments, n1 is 1.

In some embodiments,

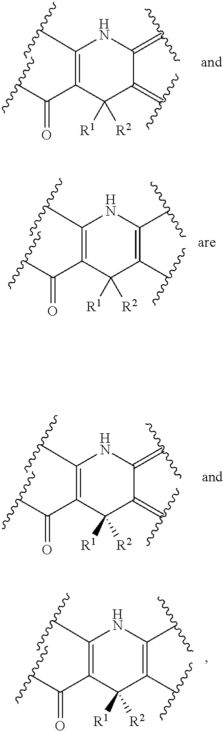

respectively. In some embodiments,

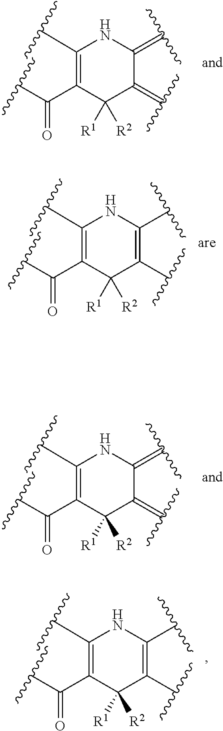

respectively.

In some embodiments, R¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl. In some embodiments, R¹ is optionally substituted C₁₋₁₀ alkyl, optionally substituted C₁₋₁₀ alkenyl, or optionally substituted C₁₋₁₀ alkynyl.

In some embodiments, R¹ is optionally substituted carbocyclyl or optionally substituted heterocyclyl. In some embodiments, R¹ is optionally substituted C₃₋₁₄ carbocyclyl. In some embodiments, R¹ is optionally substituted monocyclic C₃₋₇ carbocyclyl. In some embodiments, R¹ is optionally substituted monocyclic C₃₋₄ carbocyclyl. In some embodiments, R¹ is optionally substituted monocyclic C₅₋₇ carbocyclyl. In some embodiments, R¹ is saturated carbocyclyl. In some embodiments, R¹ is carbocyclyl comprising only one unsaturated bond (e.g., C═C bond) in the carbocyclic ring system.

In some embodiments, R¹ is 3- to 14-membered optionally substituted heterocyclyl. In some embodiments, R¹ is optionally substituted monocyclic 3- to 7-membered heterocyclyl. In some embodiments, R¹ is optionally substituted 3- to 14-membered heterocyclyl comprising one or more O and/or S atoms but no N atoms. In some embodiments, R¹ is optionally substituted 3- to 14-membered heterocyclyl comprising one or more N atoms and optionally one or more O and/S atoms. In some embodiments, R¹ is optionally substituted monocyclic 3- to 7-membered heterocyclyl comprising one or more O and/or S atoms but no N atoms. In some embodiments, R¹ is optionally substituted monocyclic 3- to 7-membered heterocyclyl comprising one or more N atoms and optionally one or more O and/or S atoms. In some embodiments, R¹ is saturated heterocyclyl. In some embodiments, R¹ is heterocyclyl comprising only one unsaturated bond (e.g., C═C bond) in the heterocyclic ring system.

In some embodiments, R¹ is optionally substituted aryl. In some embodiments, R¹ is optionally substituted monocyclic aryl. In some embodiments, R¹ is optionally substituted bicyclic aryl. In some embodiments, R¹ is optionally substituted C₆₋₁₄ aryl. In some embodiments, R¹ is optionally substituted C₆₋₁₀ aryl. In some embodiments, R¹ is optionally substituted phenyl. In some embodiments, R¹ is optionally substituted naphthyl.

In some embodiments, R¹ is optionally substituted heteroaryl. In some embodiments, R¹ is optionally substituted monocyclic heteroaryl. In some embodiments, R¹ is optionally substituted bicyclic heteroaryl. In some embodiments, R¹ is optionally substituted 5- to 14-membered heteroaryl. In some embodiments, R¹ is optionally substituted 5- to 10-membered heteroaryl. In some embodiments, R¹ is optionally substituted 5- to 6-membered monocyclic heteroaryl. In some embodiments, R¹ is optionally substituted 9- to 10-membered bicyclic heteroaryl.

In some embodiments, R¹ is optionally substituted aryl fused with optionally substituted monocyclic C₃₋₇ carbocyclyl. In some embodiments, R¹ is optionally substituted C₆₋₁₄ aryl fused with optionally substituted monocyclic C₃₋₇ carbocyclyl. In some embodiments, R¹ is optionally substituted C₆₋₁₀ aryl fused with optionally substituted monocyclic C₃₋₇ carbocyclyl. In some embodiments, R¹ is optionally substituted phenyl fused with optionally substituted monocyclic C₃₋₇ carbocyclyl. In some embodiments, R¹ is optionally substituted naphthyl fused with optionally substituted monocyclic C₃₋₇ carbocyclyl.

In some embodiments, R¹ is optionally substituted aryl fused with optionally substituted monocyclic 3- to 7-membered heterocyclyl. In some embodiments, R¹ is optionally substituted C₆₋₁₄ aryl fused with optionally substituted monocyclic 3- to 7-membered heterocyclyl. In some embodiments, R¹ is optionally substituted C₆₋₁₀ aryl fused with optionally substituted monocyclic 3- to 7-membered heterocyclyl. In some embodiments, R¹ is optionally substituted phenyl fused with optionally substituted monocyclic 3- to 7-membered heterocyclyl. In some embodiments, R¹ is optionally substituted naphthyl fused with optionally substituted monocyclic 3- to 7-membered heterocyclyl.

In some embodiments, R¹ is optionally substituted heteroaryl fused with optionally substituted monocyclic C₃₋₇ carbocyclyl. In some embodiments, R¹ is optionally substituted 5- to 14-membered heteroaryl fused with optionally substituted monocyclic C₃₋₇ carbocyclyl. In some embodiments, R¹ is optionally substituted 5- to 10-membered heteroaryl fused with optionally substituted monocyclic C₃₋₇ carbocyclyl. In some embodiments, R¹ is optionally substituted 5- to 6-membered monocyclic heteroaryl fused with optionally substituted monocyclic C₃₋₇ carbocyclyl. In some embodiments, R¹ is optionally substituted 9- to 10-membered bicyclic heteroaryl fused with optionally substituted monocyclic C₃₋₇ carbocyclyl.

In some embodiments, R¹ is optionally substituted heteroaryl fused with optionally substituted monocyclic 3- to 7-membered heterocyclyl. In some embodiments, R¹ is optionally substituted 5- to 14-membered heteroaryl fused with optionally substituted monocyclic 3- to 7-14 membered heterocyclyl. In some embodiments, R¹ is optionally substituted 5- to 10-membered heteroaryl fused with optionally substituted monocyclic 3- to 7-membered heterocyclyl. In some embodiments, R¹ is optionally substituted 5- to 6-membered monocyclic heteroaryl fused with optionally substituted monocyclic 3- to 7-membered heterocyclyl. In some embodiments, R¹ is optionally substituted 9- to 10-membered bicyclic heteroaryl fused with optionally substituted monocyclic 3- to 7-membered heterocyclyl.

In some embodiments, R¹ is

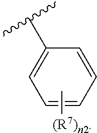

Each instance of R⁷ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(—NR^{D})R^{D}, —SC(═NR^{D})OR^{D}, —SC(═NR^{D})SR^{D}, —SC(—NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃.

n2 is 0, 1, 2, 3, 4, or 5. In some embodiments, n2 is 0, 1, 2, 3, or 4. In some embodiments, n2 is 0, 1, 2, or 3. In some embodiments, n2 is 0, 1, or 2. In some embodiments, n2 is 0 or 1. In some embodiments, n2 is 0. In some embodiments, n2 is 1. In some embodiments, n2 is 2. In some embodiments, n2 is 3. In some embodiments, n2 is 4. In some embodiments, n2 is 5.

In some embodiments, R¹ is

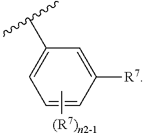

In some embodiments, R¹ is

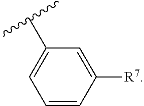

In some embodiments, R¹ is

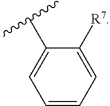

In some embodiments, R¹ is

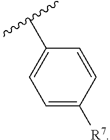

In some embodiments, R¹ is

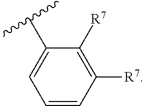

In some embodiments, R¹ is

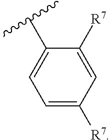

In some embodiments, R¹ is

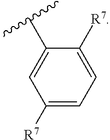

In some embodiments, R¹ is

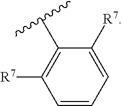

In some embodiments, R¹ is

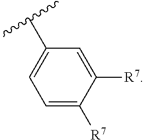

In some embodiments, R¹ is

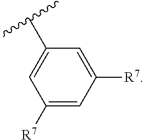

In some embodiments, R⁷ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{D}, —SR^{D}, or —N(R^{D})₂. In some embodiments, R⁷ is optionally substituted C₁₋₁₀ alkyl, optionally substituted C₁₋₁₀ alkenyl, substituted C₁₋₁₀ heteroalkenyl, or optionally substituted C₁₋₁₀ heteroalkynyl. In some embodiments, R⁷ is C₁₋₁₀ haloalkyl. In some embodiments, R⁷ is C₁₋₄ haloalkyl. In some embodiments, R⁷ is C₁₋₄ fluoroalkyl (e.g., C₁₋₄ perfluoroalkyl). In some embodiments, R⁷ is —CF₃.

In some embodiments, R⁷ is halogen. In some embodiments, R⁷ is bromine, chlorine, or fluorine. In some embodiments, R⁷ is bromine or chlorine. In some embodiments, R⁷ is chlorine or fluorine. In some embodiments, R⁷ is bromine. In some embodiments, R⁷ is chlorine. In some embodiments, R⁷ is fluorine.

In some embodiments, R⁷ is —OR^{D}, —SR^{D}, or —N(R^{D})₂ (e.g., wherein R^{D} is hydrogen or optionally substituted alkyl, or optionally substituted phenyl). In some embodiments, R⁷ is —OH. In some embodiments, R⁷ is —SH. In some embodiments, R⁷ is —NH₂. In some embodiments, R⁷ is —CN, —SCN, —SSR^{D}, —N₃, —NO, or —NO₂. In some embodiments, R⁷ is —C(═O)R^{D}, —C(═O)OR^{D}, —C(═O)SR^{D}, or —C(═O)N(R^{D})₂ (e.g., wherein R^{D} is hydrogen or optionally substituted alkyl, or optionally substituted phenyl). In some embodiments, R⁷ is —C(═NR^{D})R^{D}, —C(═NR^{D})OR^{D}, —C(═NR^{D})SR^{D}, or —C(═NR^{D})N(R^{D})₂ (e.g., wherein R^{D} is hydrogen or optionally substituted alkyl, or optionally substituted phenyl). In some embodiments, R⁷ is —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, or —S(═O)₂N(R^{D})₂ (e.g., wherein R^{D} is hydrogen or optionally substituted alkyl, or optionally substituted phenyl). In some embodiments, R⁷ is —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, or —ON(R^{D})₂ (e.g., wherein R^{D} is hydrogen or optionally substituted alkyl, or optionally substituted phenyl). In some embodiments, R⁷ is —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, or —SC(═NR^{D})N(R^{D})₂ (e.g., wherein R^{D} is hydrogen or optionally substituted alkyl, or optionally substituted phenyl). In some embodiments, R⁷ is —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, or —NR^{D}S(═O)₂N(R^{D})₂ (e.g., wherein R^{D} is hydrogen or optionally substituted alkyl, or optionally substituted phenyl). In some embodiments, R⁷ is —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃ (e.g., wherein R^{D} is hydrogen or optionally substituted alkyl, or optionally substituted phenyl).

In some embodiments, R⁷ is optionally substituted carbocyclyl, optionally substituted heterocyclyl, optionally substituted aryl, or optionally substituted heteroaryl. In some embodiments, R⁷ is optionally substituted C₃₋₁₄ carbocyclyl. In some embodiments, R⁷ is optionally substituted monocyclic C₃₋₇ carbocyclyl. In some embodiments, R⁷ is optionally substituted monocyclic C₃₋₄ carbocyclyl. In some embodiments, R⁷ is optionally substituted monocyclic C₅₋₇ carbocyclyl. In some embodiments, R⁷ is saturated carbocyclyl. In some embodiments, R⁷ is carbocyclyl comprising only one unsaturated bond (e.g., C═C bond) in the carbocyclic ring system.

In some embodiments, R⁷ is optionally substituted 3- to 14-membered heterocyclyl. In some embodiments, R² is optionally substituted monocyclic 3- to 7-membered heterocyclyl. In some embodiments, R¹ is optionally substituted 3- to 14-membered heterocyclyl comprising one or more O and/or S atoms but no N atoms. In some embodiments, R⁷ is optionally substituted 3- to 14-membered heterocyclyl comprising one or more N atoms and optionally one or more O and/S atoms. In some embodiments, R⁷ is optionally substituted monocyclic 3- to 7-membered heterocyclyl comprising one or more O and/or S atoms but no N atoms. In some embodiments, R⁷ is optionally substituted monocyclic 3- to 7-membered heterocyclyl comprising one or more N atoms and optionally one or more O and/or S atoms. In some embodiments, R⁷ is saturated heterocyclyl. In some embodiments, R⁷ is heterocyclyl comprising only one unsaturated bond (e.g., C═C bond) in the heterocyclic ring system.

In some embodiments, R⁷ is optionally substituted monocyclic aryl. In some embodiments, R⁷ is optionally substituted bicyclic aryl. In some embodiments, R⁷ is optionally substituted C₆₋₁₄ aryl. In some embodiments, R⁷ is optionally substituted C₆₋₁₀ aryl. In some embodiments, R⁷ is optionally substituted phenyl. In some embodiments, R⁷ is optionally substituted naphthyl.

In some embodiments, R⁷ is optionally substituted monocyclic heteroaryl. In some embodiments, R⁷ is optionally substituted bicyclic heteroaryl. In some embodiments, R⁷ is optionally substituted 5- to 14-membered heteroaryl. In some embodiments, R⁷ is optionally substituted 5- to 10-membered heteroaryl. In some embodiments, R⁷ is optionally substituted 5- to 6-membered monocyclic heteroaryl. In some embodiments, R⁷ is optionally substituted heteroaryl comprising one or more N atoms. In some embodiments, R⁷ is optionally substituted pyridinyl, optionally substituted isoquinolinyl, optionally substituted quinolinyl, or optionally substituted pyrazolyl. In some embodiments, R⁷ is optionally substituted pyridinyl or optionally substituted pyrazolyl.

In some embodiments, at least one instance of R^{D} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{D} is optionally substituted carbocyclyl, optionally substituted heterocyclyl, optionally substituted aryl, or optionally substituted heteroaryl. In some embodiments, at least one instance of R^{D} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{D} is independently hydrogen, optionally substituted C₁₋₁₀ alkyl, or optionally substituted phenyl. In some embodiments, at least one instance of R^{D} is a nitrogen protecting group when attached to a nitrogen atom, an oxygen protecting group when attached to an oxygen atom, or a sulfur protecting group when attached to a sulfur atom. In some embodiments, two instances of R^{D} attached to the same intervening atom are joined together with the intervening atom to form an optionally substituted, monocyclic, heterocyclic or heteroaryl ring.

In some embodiments, R² is optionally substituted alkyl, optionally substituted alkenyl, or optionally substituted alkynyl. In some embodiments, R² is optionally substituted C₁₋₄ alkyl, optionally substituted C₁₋₄ alkenyl, or optionally substituted C₁₋₄ alkynyl. In some embodiments, R² is optionally substituted alkyl. In some embodiments, R² is optionally substituted C₁₋₄ alkyl. In some embodiments, R² is unsubstituted C₁-C₄ alkyl. In some embodiments, R² is unsubstituted methyl. In some embodiments, R² is unsubstituted ethyl.

In some embodiments, R¹ and R² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring. In some embodiments, R¹ and R² are taken together with their intervening atom to form an optionally substituted, monocyclic, carbocyclic ring. In some embodiments, R¹ and R² are taken together with their intervening atom to form an optionally substituted, monocyclic, 3- to 7-membered carbocyclic ring. In some embodiments, R¹ and R² are taken together with their intervening atom to form an optionally substituted, monocyclic, 5- to 6-membered carbocyclic ring. In some embodiments, R¹ and R² are taken together with their intervening atom to form an optionally substituted, monocyclic, heterocyclic ring. In some embodiments, R¹ and R² are taken together with their intervening atom to form an optionally substituted, monocyclic, 3- to 7-membered heterocyclic ring. In some embodiments, R¹ and R² are taken together with their intervening atom to form an optionally substituted, monocyclic, 5- to 6-membered heterocyclic ring. In some embodiments, R¹ and R² are taken together with their intervening atom to form an optionally substituted, monocyclic, heterocyclic ring comprising one or more N atoms. In some embodiments, R¹ and R² are taken together with their intervening atom to form an optionally substituted, monocyclic, 3- to 7-membered heterocyclic ring comprising one or more N atoms. In some embodiments, R¹ and R² are taken together with their intervening atom to form optionally substituted piperidinyl. In some embodiments, R¹ and R² are taken together with their intervening atom to form

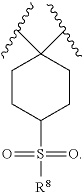

R⁸ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a sulfur protecting group. In some embodiments, R⁸ is hydrogen, optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, optionally substituted C₁₋₁₀ heteroalkynyl, optionally substituted C₃₋₁₄ carbocyclyl, optionally substituted 3- to 14-membered heterocyclyl, optionally substituted C₆₋₁₄ aryl, optionally substituted 5-14-membered heteroaryl, or a sulfur protecting group.

In some embodiments, R³ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{A}, —SR^{A}, or —N(R^{A})₂. In some embodiments, R³ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R³ is Cie haloalkyl. In some embodiments, R³ is C₁₋₄ haloalkyl. In some embodiments, R³ is C₁₋₄ fluoroalkyl (e.g., C₁₋₄ perfluoroalkyl). In some embodiments, R³ is —CF₃. In some embodiments, R³ is hydrogen, optionally substituted C₁-C₆ alkyl, or halogen. In some embodiments, R³ is hydrogen, fluorine, —CH₃, —CH₂F, —CHF₂, or —CF₃. In some embodiments, R³ is hydrogen, fluorine, —CH₃, or —CF₃. In some embodiments, R³ is hydrogen or halogen. In some embodiments, R³ is hydrogen or fluorine. In some embodiments, R³ is hydrogen or optionally substituted C₁-C₆ alkyl. In some embodiments, R³ is hydrogen, unsubstituted C₁₋₆ alkyl, or C₁₋₆ haloalkyl. In some embodiments, R³ is hydrogen.

In some embodiments, R³ is halogen. In some embodiments, R³ is bromine, chlorine, or fluorine. In some embodiments, R³ is bromine or chlorine. In some embodiments, R³ is chlorine or fluorine. In some embodiments, R³ is bromine. In some embodiments, R³ is chlorine. In some embodiments, R³ is fluorine.

In some embodiments, R³ is —OR^{A}, —SR^{A}, or —N(R^{A})₂ (e.g., wherein R^{A} is hydrogen or optionally substituted alkyl, or optionally substituted phenyl). In some embodiments, R³ is —OH. In some embodiments, R³ is —SH. In some embodiments, R³ is —NH₂. In some embodiments, R³ is —CN, —SCN, —SSR^{A}, —N₃, —NO, or —NO₂. In some embodiments, R³ is —C(═O)R^{A}, —C(═O)OR^{A}, —C(═O)SR^{A}, or —C(═O)N(R^{A}), (e.g., wherein R^{A} is hydrogen or optionally substituted alkyl, or optionally substituted phenyl). In some embodiments, R³ is —C(═NR^{A})R^{A}, —C(═NR^{A})OR^{A}, —C(═NR^{A})SR^{A}, or —C(═NR^{A})N(R^{A}), (e.g., wherein R^{A} is hydrogen or optionally substituted alkyl, or optionally substituted phenyl). In some embodiments, R³ is —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, or —S(═O)₂N(R^{A})₂ (e.g., wherein R^{A} is hydrogen or optionally substituted alkyl, or optionally substituted phenyl). In some embodiments, R³ is —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, or —ON(R^{A})₂ (e.g., wherein R^{A} is hydrogen or optionally substituted alkyl, or optionally substituted phenyl). In some embodiments, R³ is —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, or —SC(═NR^{A})N(R^{A})₂ (e.g., wherein R^{A} is hydrogen or optionally substituted alkyl, or optionally substituted phenyl). In some embodiments, R³ is —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, or —NR^{A}S(═O)₂N(R^{A})₂ (e.g., wherein R^{A} is hydrogen or optionally substituted alkyl, or optionally substituted phenyl). In some embodiments, R³ is —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, or —OSi(OR^{A})₃ (e.g., wherein R^{A} is hydrogen or optionally substituted alkyl, or optionally substituted phenyl).

In some embodiments, R³ is optionally substituted carbocyclyl, optionally substituted heterocyclyl, optionally substituted aryl, or optionally substituted heteroaryl. In some embodiments, R³ is optionally substituted C₃₋₁₄ carbocyclyl. In some embodiments, R³ is optionally substituted monocyclic C₃₋₇ carbocyclyl. In some embodiments, R³ is optionally substituted monocyclic C₃₋₄ carbocyclyl. In some embodiments, R³ is optionally substituted monocyclic C₅₋₇ carbocyclyl. In some embodiments, R³ is saturated carbocyclyl. In some embodiments, R³ is carbocyclyl comprising only one unsaturated bond (e.g., C═C bond) in the carbocyclic ring system.

In some embodiments, R³ is optionally substituted 3- to 14-membered heterocyclyl. In some embodiments, R³ is optionally substituted monocyclic 3- to 7-membered heterocyclyl. In some embodiments, R³ is optionally substituted 3- to 14-membered heterocyclyl comprising one or more O and/or S atoms but no N atoms. In some embodiments, R³ is optionally substituted 3- to 14-membered heterocyclyl comprising one or more N atoms and optionally one or more O and/S atoms. In some embodiments, R³ is optionally substituted monocyclic 3- to 7-membered heterocyclyl comprising one or more O and/or S atoms but no N atoms. In some embodiments, R³ is optionally substituted monocyclic 3- to 7-membered heterocyclyl comprising one or more N atoms and optionally one or more O and/or S atoms. In some embodiments, R³ is saturated heterocyclyl. In some embodiments, R³ is heterocyclyl comprising only one unsaturated bond (e.g., C═C bond) in the heterocyclic ring system.

In some embodiments, R³ is optionally substituted monocyclic aryl. In some embodiments, R³ is optionally substituted bicyclic aryl. In some embodiments, R³ is optionally substituted C₆₋₁₄ aryl. In some embodiments, R³ is optionally substituted C₆₋₁₀ aryl. In some embodiments, R³ is optionally substituted phenyl. In some embodiments, R³ is optionally substituted naphthyl.

In some embodiments, R³ is optionally substituted monocyclic heteroaryl. In some embodiments, R³ is optionally substituted bicyclic heteroaryl. In some embodiments, R³ is optionally substituted 5- to 14-membered heteroaryl. In some embodiments, R³ is optionally substituted 5- to 10-membered heteroaryl. In some embodiments, R³ is optionally substituted 5- to 6-membered monocyclic heteroaryl.

In some embodiments, at least one instance of R^{A} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{A} is optionally substituted carbocyclyl, optionally substituted heterocyclyl, optionally substituted aryl, or optionally substituted heteroaryl. In some embodiments, at least one instance of R^{A} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{A} is independently hydrogen, optionally substituted C₁₋₁₀ alkyl, or optionally substituted phenyl. In some embodiments, at least one instance of R^{A} is a nitrogen protecting group when attached to a nitrogen atom, an oxygen protecting group when attached to an oxygen atom, or a sulfur protecting group when attached to a sulfur atom. In some embodiments, two instances of R^{A} attached to the same intervening atom are joined together with the intervening atom to form an optionally substituted, monocyclic, heterocyclic or heteroaryl ring.

In some embodiments, at least one of R^{4a} and R^{4b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, at least one of R^{4a} and R^{4b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, at least one of R^{4a} and R^{4b} is independently hydrogen, halogen, or optionally substituted alkyl. In some embodiments, each of R^{4a} and R^{4b} is independently hydrogen, halogen, or optionally substituted alkyl. In some embodiments, at least one of R^{4a} and R^{4b} is independently hydrogen, halogen, or optionally substituted C₁₋₄ alkyl. In some embodiments, at least one of R^{4a} and R^{4b} is independently hydrogen, fluorine, or optionally substituted C₁₋₄ alkyl. In some embodiments, at least one of R^{4a} and R^{4b} is independently hydrogen or halogen. In some embodiments, at least one of R^{4a} and R^{4b} is independently hydrogen or fluorine. In some embodiments, at least one of R^{4a} and R^{4b} is independently hydrogen or optionally substituted C₁₋₄ alkyl. In some embodiments, at least one of R^{4a} and R^{4b} is hydrogen. In some embodiments, at least one of R^{4a} and R^{4b} is hydrogen. In some embodiments, at least one of R^{4a} and R^{4b} is optionally substituted C₁₋₄ alkyl. In some embodiments, at least one of R^{4a} and R^{4b} is unsubstituted methyl. In some embodiments, at least one of R^{4a} and R^{4b} is optionally substituted C₁₋₄ alkyl. In some embodiments, at least one of R^{4a} and R^{4b} is unsubstituted methyl. In some embodiments, at least one of R^{4a} and R^{4b} is fluorine.

In some embodiments, R^{4a} and R^{4b} are taken together with their intervening atom to form an optionally substituted, monocyclic, carbocyclic or heterocyclic ring. In some embodiments, R^{4a} and R^{4b} are taken together with their intervening atom to form an optionally substituted, monocyclic, C₃₋₁₄ carbocyclic ring. In some embodiments, R^{4a} and R^{4b} are taken together with their intervening atom to form an optionally substituted, monocyclic, C₃₋₇ carbocyclic ring. In some embodiments, R^{4a} and R^{4b} are taken together with their intervening atom to form an optionally substituted, monocyclic, 3- to 14-membered heterocyclic ring. In some embodiments, R^{4a} and R^{4b} are taken together with their intervening atom to form an optionally substituted, monocyclic, 3- to 7-membered heterocyclic ring.

In some embodiments, R^{4b} and R^{5a} are taken together with their intervening atom to form an optionally substituted, monocyclic, carbocyclic, heterocyclic, aryl, or heteroaryl ring. In some embodiments, R^{4b} and R^{5a} are taken together with their intervening atom to form an optionally substituted, monocyclic, carbocyclic or heterocyclic ring. In some embodiments, R^{4b} and R^{5a} are taken together with their intervening atom to form an optionally substituted, monocyclic, C₃₋₁₄ carbocyclic ring. In some embodiments, R^{4b} and R^{5a} are taken together with their intervening atom to form an optionally substituted, monocyclic, C₃₋₇ carbocyclic ring. In some embodiments, R^{4b} and R^{5a} are taken together with their intervening atom to form an optionally substituted, monocyclic, 3- to 14-membered heterocyclic ring. In some embodiments, R^{4b} and R^{5a} are taken together with their intervening atom to form an optionally substituted, monocyclic, 3- to 7-membered heterocyclic ring. In some embodiments, R^{4b} and R^{5a} are taken together with their intervening atom to form an optionally substituted, monocyclic, aryl, or heteroaryl ring. In some embodiments, R^{4b} and R^{5a} are taken together with their intervening atom to form an optionally substituted phenyl ring. In some embodiments, R^{4b} and R^{5a} are taken together with their intervening atom to form an optionally substituted, monocyclic, 5- or 6-membered heteroaryl ring.

In some embodiments, at least one of R^{5a} and R^{5b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, at least one of R^{5a} and R^{5b} is independently hydrogen, halogen, or optionally substituted alkyl. In some embodiments, each of R^{5a} and R^{5b} is independently hydrogen, halogen, or optionally substituted alkyl. In some embodiments, at least one of R^{5a} and R^{5b} is independently hydrogen, —CH₃, or —CF₃. In some embodiments, at least one of R^{5a} and R^{5b} is hydrogen. In some embodiments, at least one of R^{5a} and R^{5b} is independently optionally substituted C₁₋₁₀ alkyl, optionally substituted C₁₋₁₀ alkenyl, or optionally substituted C₁₋₁₀ alkynyl. In some embodiments, at least one of R^{5a} and R^{5b} is independently optionally substituted C₁₋₁₀ heteroalkyl, optionally substituted C₁₋₁₀ heteroalkenyl, or optionally substituted C₁₋₁₀ heteroalkynyl. In some embodiments, at least one of R^{5a} and R^{5b} is independently optionally substituted C₁₋₁₀ alkyl or optionally substituted C₁₋₁₀ heteroalkyl. In some embodiments, at least one of R^{5a} and R^{5b} is optionally substituted C₁₋₄ alkyl. In some embodiments, at least one of R^{5a} and R^{5b} is —CH₃. In some embodiments, at least one of R^{5a} and R^{5b} is —CF₃. In some embodiments, at least one of R^{5a} and R^{5b} is optionally substituted C₁₋₄ heteroalkyl. In some embodiments, at least one of R^{5a} and R^{5b} is optionally substituted C₁₋₄ heteroalkyl comprising an O atom. In some embodiments, at least one of R^{5a} and R^{3b} is optionally substituted C₆₋₁₄ aryl. In some embodiments, at least one of R^{5a} and R^{5b} is optionally substituted C₆₋₁₀ aryl. In some embodiments, at least one of R^{5a} and R^{5b} is optionally substituted phenyl. In some embodiments, R^{5a} and R^{5b} are —CH₃. In some embodiments, R^{5a} and R^{5b} are hydrogen.

In some embodiments, R^{5a} and R^{5b} are taken together with their intervening atom to form an optionally substituted, monocyclic, carbocyclic or heterocyclic ring. In some embodiments, R^{5a} and R^{5b} are taken together with their intervening atom to form an optionally substituted, monocyclic, carbocyclic ring. In some embodiments, R^{5a} and R^{5b} are taken together with their intervening atom to form an optionally substituted, monocyclic, C₃₋₁₄ carbocyclic ring. In some embodiments, R^{5a} and R^{5b} are taken together with their intervening atom to form an optionally substituted, monocyclic, C₃₋₇ carbocyclic ring. In some embodiments, R^{5a} and R^{5b} are taken together with their intervening atom to form an optionally substituted cyclopropyl, cyclobutyl, cyclopentyl, or cyclohexyl ring. In some embodiments, R^{5a} and R^{5b} are taken together with their intervening atom to form an optionally substituted, monocyclic, heterocyclic ring. In some embodiments, R^{5a} and R^{5b} are taken together with their intervening atom to form an optionally substituted, monocyclic, heterocyclic ring comprising O and/or S heteroatom(s) as the only heteroatoms in the heterocyclic ring. In some embodiments, R^{5a} and R^{5b} are taken together with their intervening atom to form an optionally substituted, monocyclic, heterocyclic ring comprising O and/or N heteroatom(s) as the only heteroatoms in the heterocyclic ring. In some embodiments, R^{5a} and R^{5b} are taken together with their intervening atom to form an optionally substituted, monocyclic, 3- to 14-membered heterocyclic ring. In some embodiments, R^{5a} and R^{5b} are taken together with their intervening atom to form an optionally substituted, monocyclic, 3- to 7-membered heterocyclic ring. In some embodiments, R^{5a} and R^{5b} are taken together with their intervening atom to form an optionally substituted, monocyclic, 5- to 6-membered heterocyclic ring. In some embodiments, R^{5a} and R^{5b} are taken together with their intervening atom to form optionally substituted tetrahydrofuran, optionally substituted tetrahydropyran, optionally substituted pyrrolidine.

In some embodiments, R⁶ is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl. In some embodiments, R⁶ is hydrogen. In some embodiments, R⁶ is optionally substituted C₁₋₁₀ alkyl, optionally substituted C₁₋₁₀ alkenyl, or C₁₋₁₀ alkynyl. In some embodiments, R⁶ is optionally substituted alkyl. In some embodiments, R⁶ is optionally substituted C₁₋₁₀ alkyl. In some embodiments, R⁶ is optionally substituted C₁₋₆ alkyl. In some embodiments, R⁶ is optionally substituted C₁₋₄ alkyl. In some embodiments, R⁶ is unsubstituted methyl. In some embodiments, R⁶ is benzyl.

In some embodiments, R⁶ is optionally substituted carbocyclyl or optionally substituted heterocyclyl. In some embodiments, R⁶ is optionally substituted C₃₋₁₄ carbocyclyl. In some embodiments, R⁶ is optionally substituted monocyclic C₃₋₇ carbocyclyl. In some embodiments, R⁶ is optionally substituted monocyclic C₅₋₇ carbocyclyl. In some embodiments, R⁶ is saturated carbocyclyl. In some embodiments, R⁶ is carbocyclyl comprising only one unsaturated bond (e.g., C═C bond) in the carbocyclic ring system.

In some embodiments, R⁶ is optionally substituted 3- to 14-membered heterocyclyl. In some embodiments, R⁶ is optionally substituted monocyclic 3- to 7-membered heterocyclyl. In some embodiments, R⁶ is optionally substituted heterocyclyl comprising one or more O and/or S atoms, but no N atoms. In some embodiments, R⁶ is optionally substituted 3- to 14-membered heterocyclyl comprising one or more N atoms and optionally one or more O and/S atoms. In some embodiments, R⁶ is optionally substituted monocyclic 3- to 7-membered heterocyclyl comprising one or more O and/or S atoms but no N atoms. In some embodiments, R⁶ is optionally substituted monocyclic 3- to 7-membered heterocyclyl comprising one or more N atoms and optionally one or more O and/or S atoms. In some embodiments, R⁶ is saturated heterocyclyl. In some embodiments, R⁶ is heterocyclyl comprising only one unsaturated bond (e.g., C═C bond) in the heterocyclic ring system.

In some embodiments, R⁶ is optionally substituted aryl or optionally substituted heteroaryl. In some embodiments, R⁶ is optionally substituted monocyclic aryl. In some embodiments, R⁶ is optionally substituted bicyclic aryl. In some embodiments, R⁶ is optionally substituted C₆₋₁₄ aryl. In some embodiments, R⁶ is optionally substituted C₆₋₁₀ aryl. In some embodiments, R⁶ is optionally substituted phenyl. In some embodiments, R⁶ is optionally substituted naphthyl.

In some embodiments, R⁶ is optionally substituted monocyclic heteroaryl. In some embodiments, R⁶ is optionally substituted bicyclic heteroaryl. In some embodiments, R⁶ is optionally substituted 5- to 14-membered heteroaryl. In some embodiments, R⁶ is optionally substituted 5- to 10-membered heteroaryl. In some embodiments, R⁶ is optionally substituted 5- to 6-membered monocyclic heteroaryl. In some embodiments, R⁶ is optionally substituted heteroaryl comprising one or more N atoms. In some embodiments, R⁶ is optionally substituted pyridyl, optionally substituted phenyl, optionally substituted pyrimidinyl, optionally substituted imidazolyl, optionally substituted pyrazolyl, or optionally substituted isothiazolyl. In some embodiments, R⁶ is optionally substituted pyridyl, optionally substituted phenyl, or optionally substituted pyrimidinyl.

In some embodiments, Formula I-A or I-B is any one of the formulae shown in Table 1:

TABLE 1

| | |
|---|---|
| 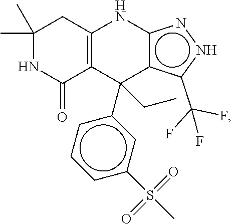 | 947799 |
| 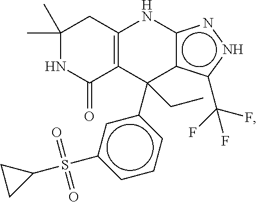 | 947859 |
| 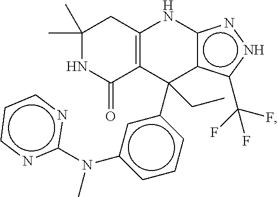 | 948176 |
| 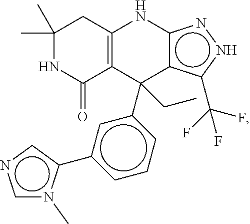 | 948180 |
| 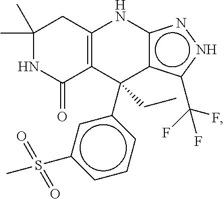 | 948418 |
| 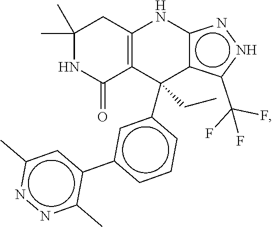 | 949917 |
| 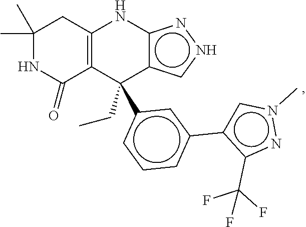 | 950563 |
| 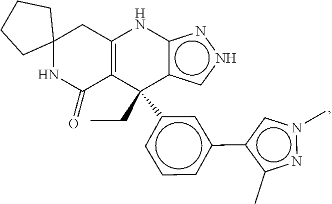 | 950252 |
| 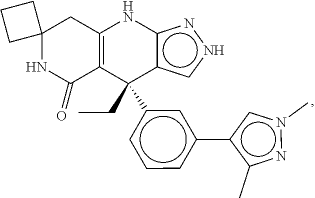 | 950055 |
| 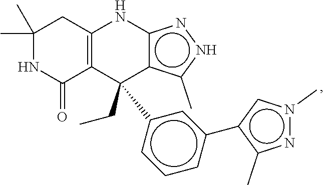 | 946001 |
| 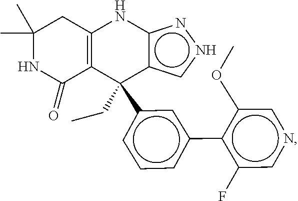 | 948150 |
| 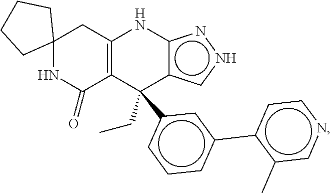 | 948022 |
| 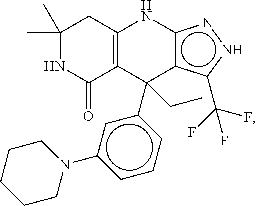 | 947861 |
| 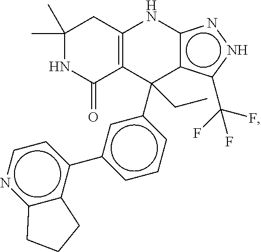 | 947862 |
| 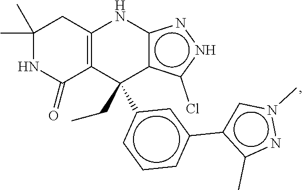 | 949336 |
| 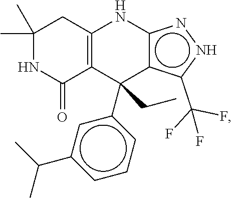 | 950494 |
| 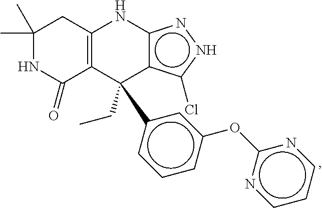 | 951606 |
| 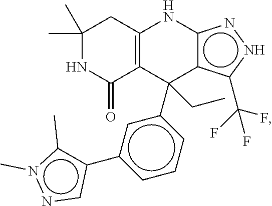 | 947403 |
| 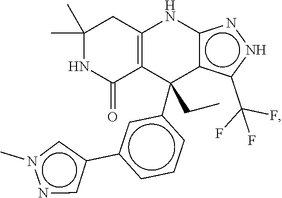 | 947877 |
| 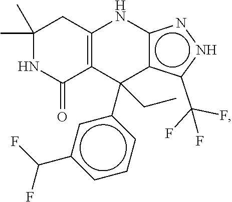 | 948305 |
| 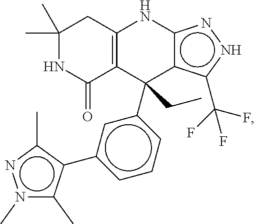 | 948421 |
| 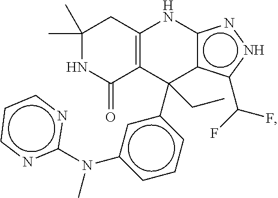 | 950484 |
| 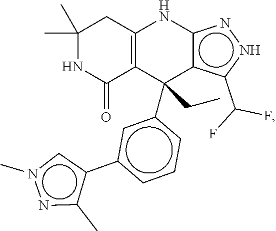 | 950497 |
| 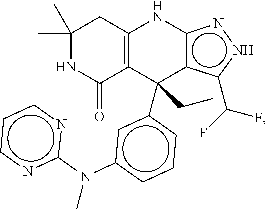 | 951740 |
| 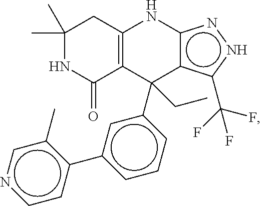 | 947404 |
| 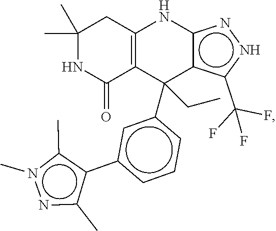 | 947568 |
| 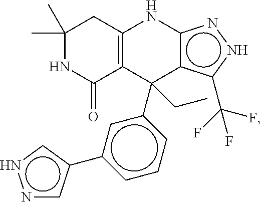 | 947677 |
| 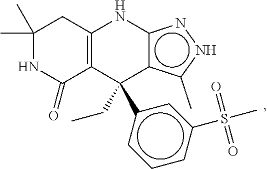 | 947811 |
| 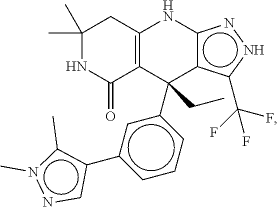 | 947819 |
| 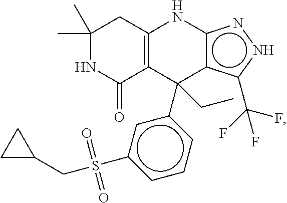 | 948179 |
| 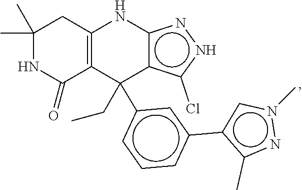 | 948768 |
| 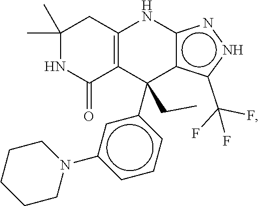 | 949183 |
| 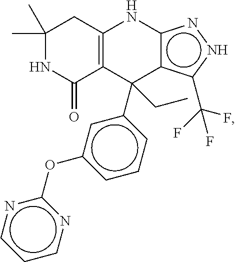 | 950323 |
| 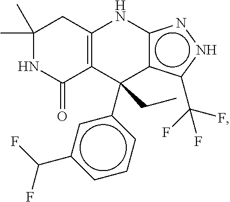 | 951948 |
| 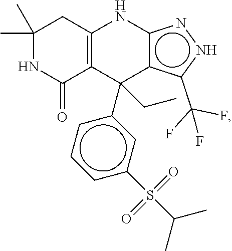 | 947800 |
| 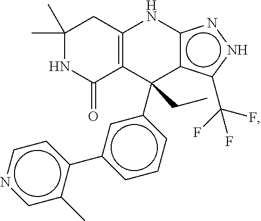 | 947814 |
| 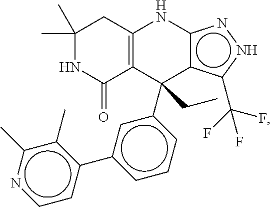 | 948185 |
| 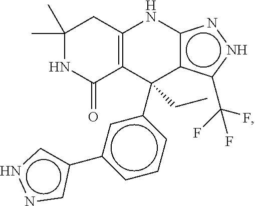 | 948307 |
| 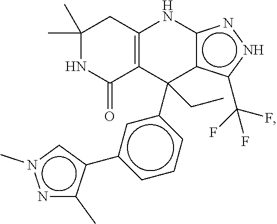 | 948314 |
| 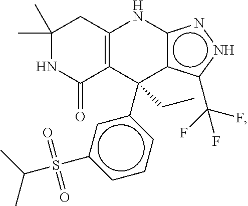 | 948419 |
| 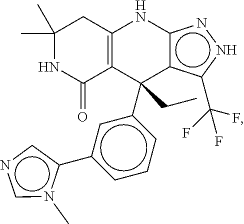 | 949010 |
| 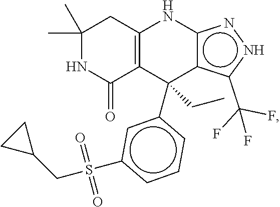 | 949175 |
| 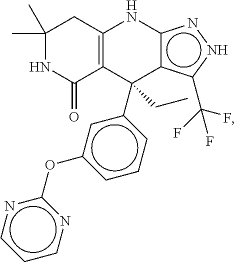 | 951607 |
| 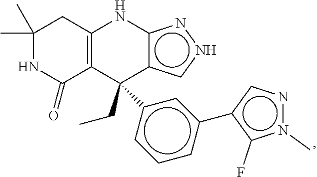 | 949395 |
| 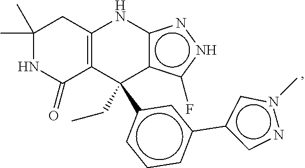 | 949002 |
| 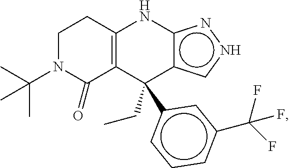 | 945634 |
| 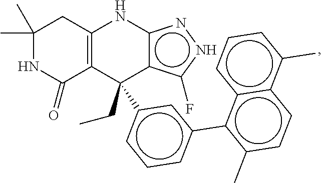 | 949392 |
| 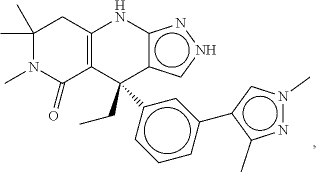 | 949008 |
| 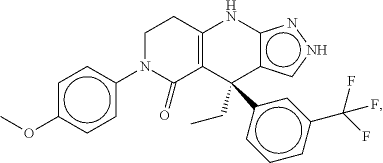 | 948227 |
| 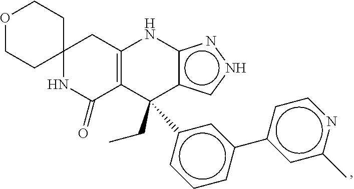 | 948524 |
| 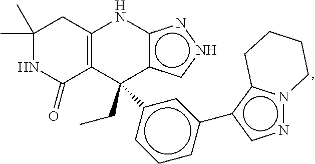 | 947415 |
| 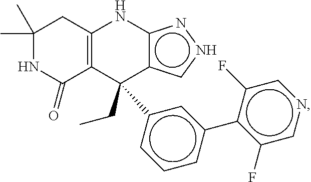 | 948514 |
| 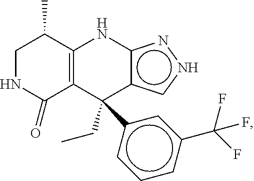 | 945384 |
| 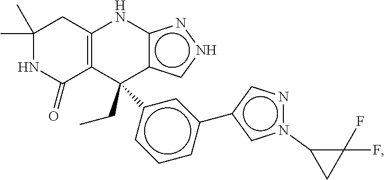 | 949393 |
| 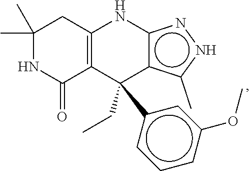 | 949328 |
| 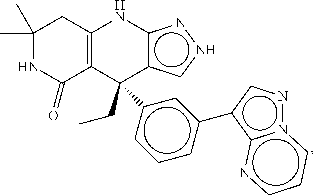 | 949389 |
| 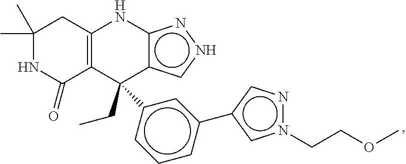 | 949114 |
| 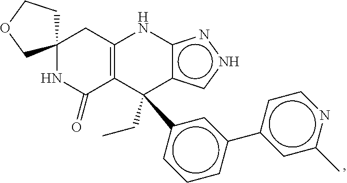 | 948148 |
| 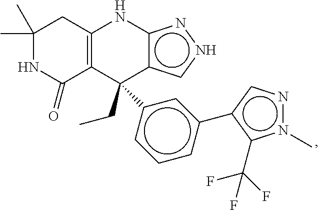 | 948826 |
| 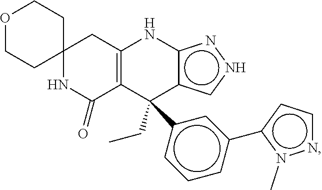 | 948526 |
| 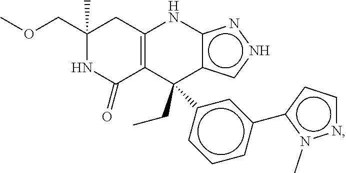 | 947907 |
| 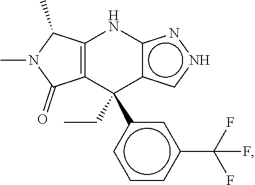 | 913475 |
| 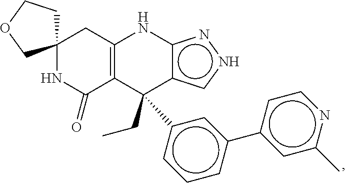 | 948141 |
| 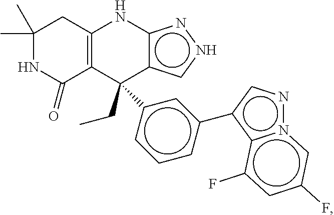 | 949390 |
| 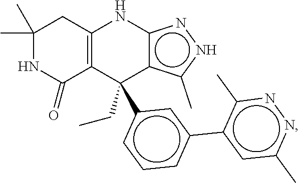 | 949332 |
| 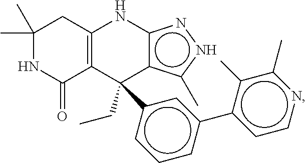 | 948183 |
| 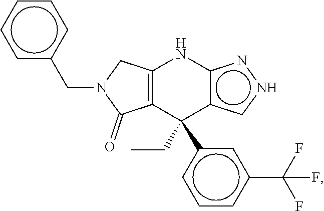 | 929389 |
| 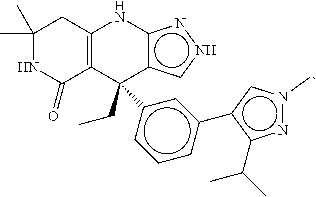 | 949106 |
| 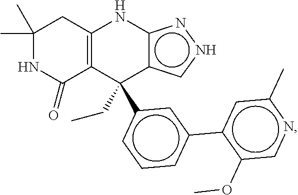 | 949512 |
| 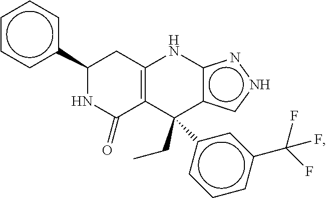 | 945362 |
| 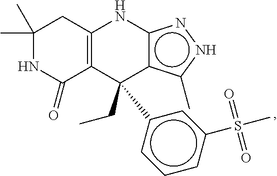 | 947815 |
| 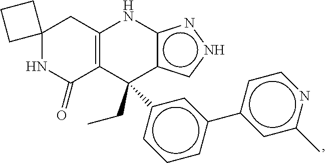 | 949517 |
| 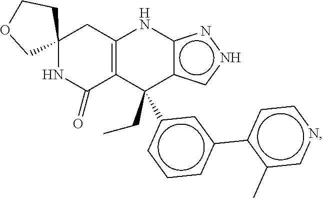 | 948662 |
| 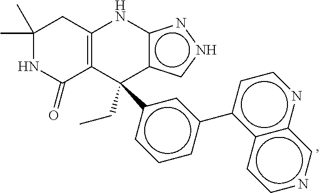 | 949110 |
| 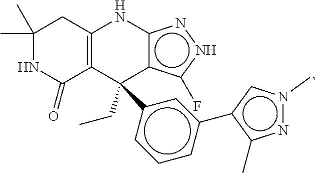 | 949176 |
| 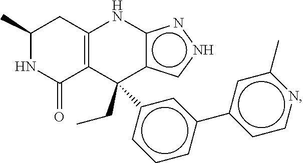 | 926299 |
| 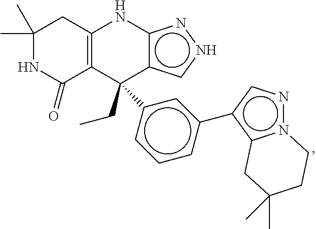 | 949265 |
| 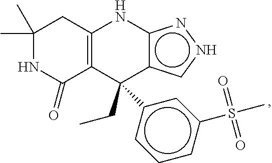 | 945621 |
| 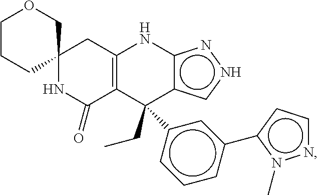 | 948365 |
| 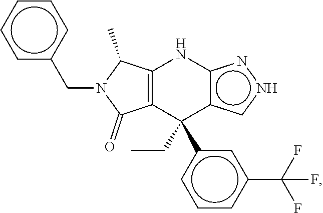 | 930332 |
| 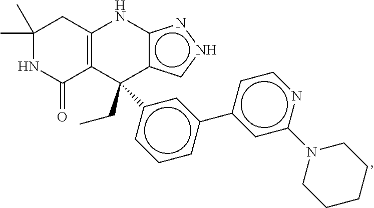 | 948646 |
| 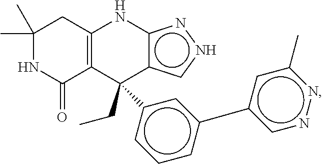 | 944071 |
| 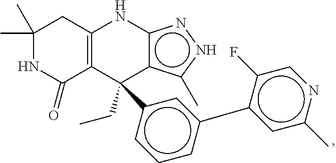 | 949327 |
| 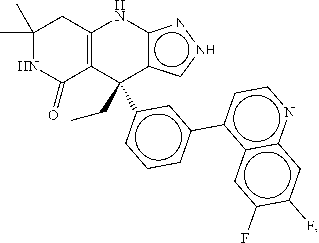 | 948664 |
| 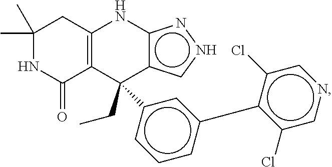 | 948824 |
| 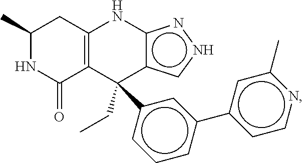 | 926302 |
| 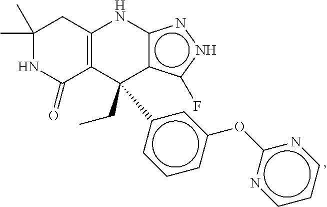 | 951944 |
| 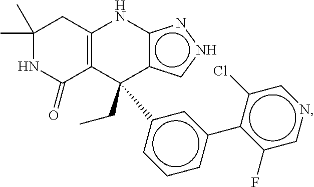 | 948363 |
| 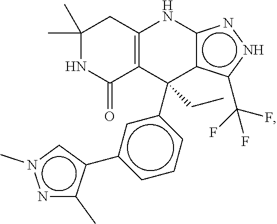 | 948309 |
| 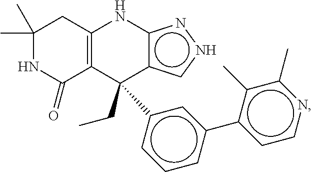 | 948188 |
| 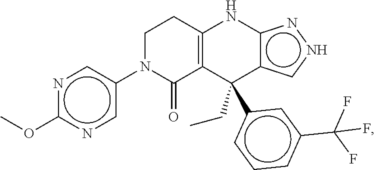 | 949127 |
| 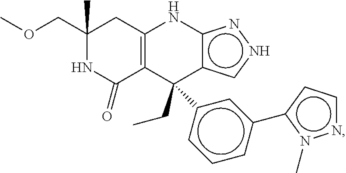 | 947908 |
| 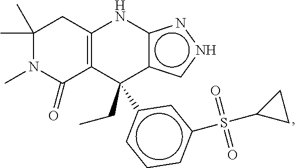 | 949184 |
| 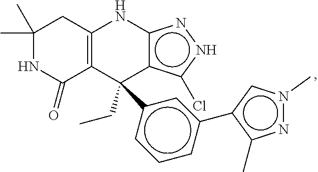 | 949329 |
| 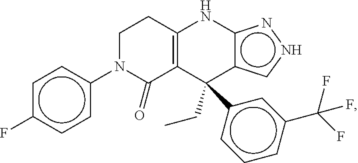 | 948225 |
| 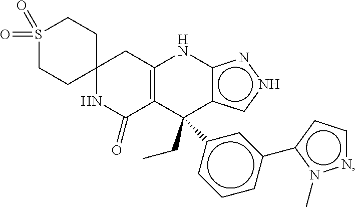 | 949400 |
| 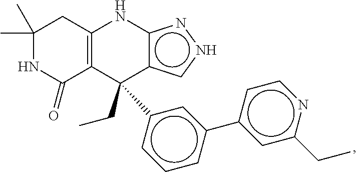 | 948360 |
| 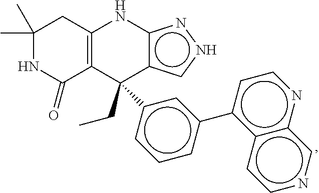 | 948819 |
| 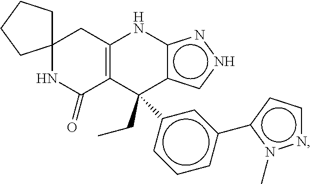 | 948019 |
| 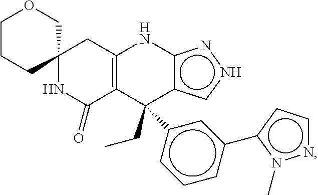 | 948366 |
| 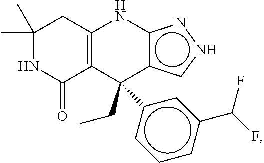 | 949007 |
| 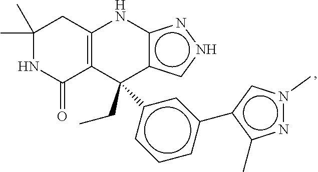 | 945480 |
| 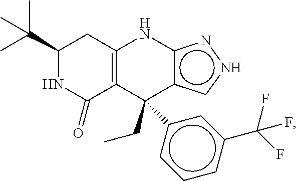 | 945921 |
| 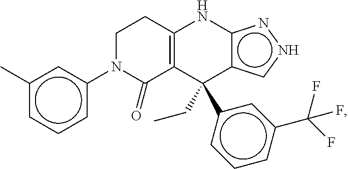 | 949041 |
| 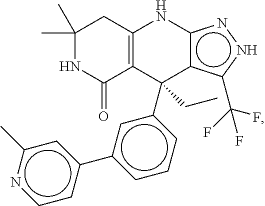 | 948773 |
| 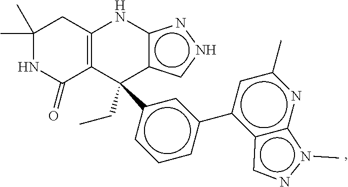 | 952563 |
| 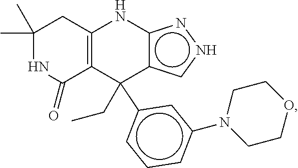 | 945602 |
| 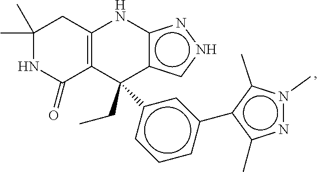 | 947413 |
| 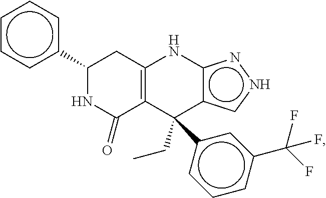 | 945363 |
| 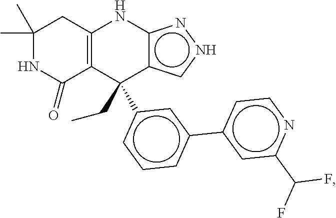 | 949388 |
| 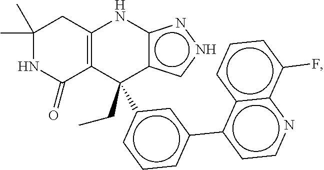 | 948813 |
| 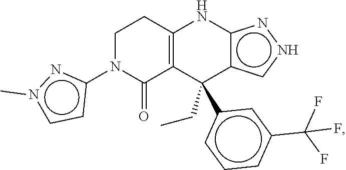 | 949836 |
| 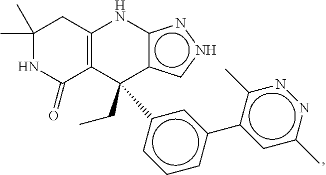 | 949334 |
| 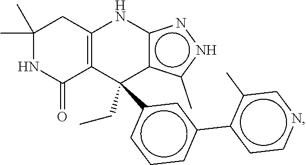 | 947409 |
| 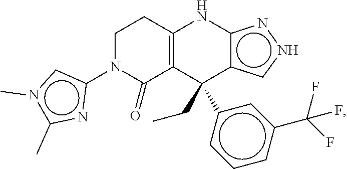 | 952478 |
| 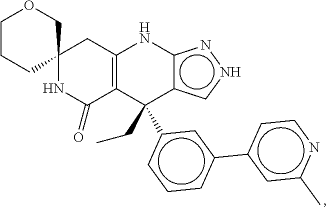 | 948146 |
| 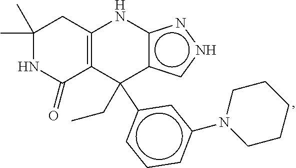 | 947566 |
| 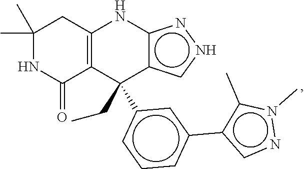 | 945617 |
| 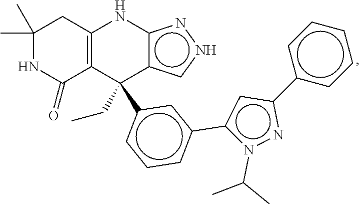 | 952411 |
| 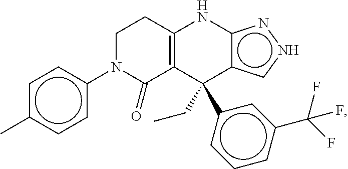 | 949119 |
| 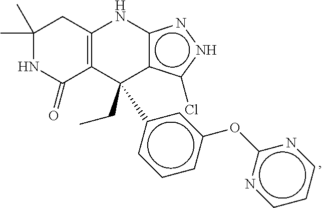 | 951605 |
| 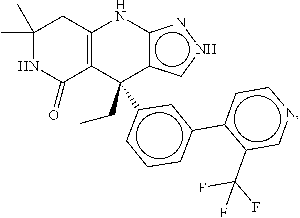 | 949516 |
| 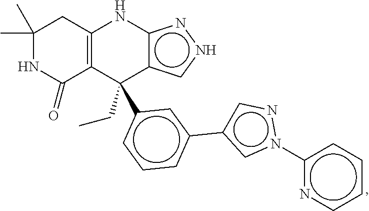 | 952566 |
| 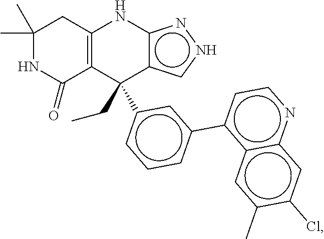 | 948645 |
| 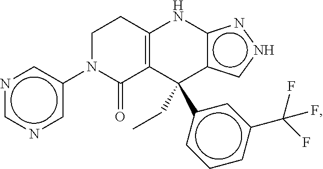 | 948380 |
| 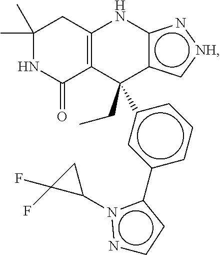 | 949518 |
| 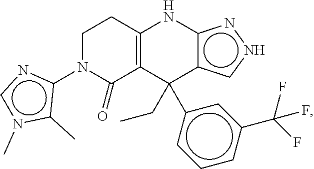 | 951598 |
| 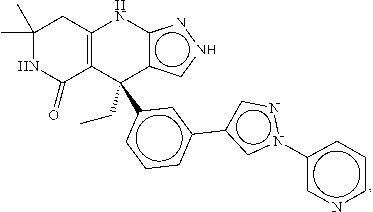 | 952024 |
| 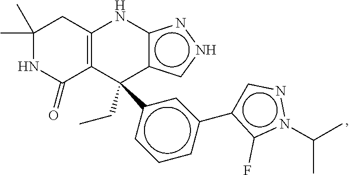 | 950061 |
| 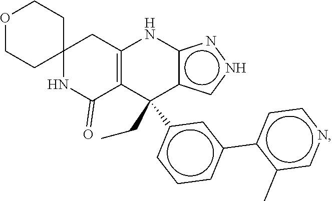 | 948367 |
| 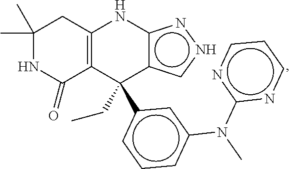 | 949179 |
| 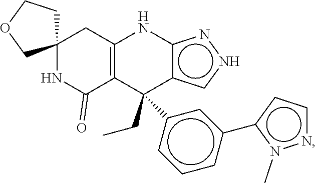 | 948014 |
| 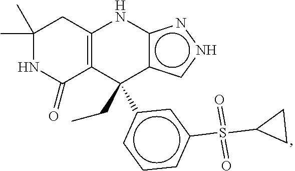 | 946004 |
| 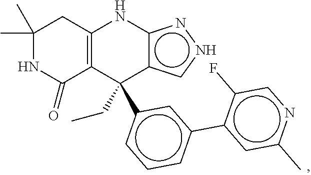 | 947822 |
| 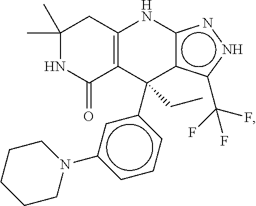 | 949178 |
| 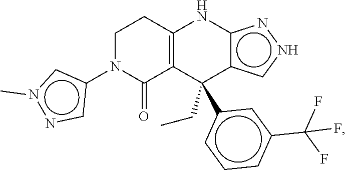 | 949271 |
| 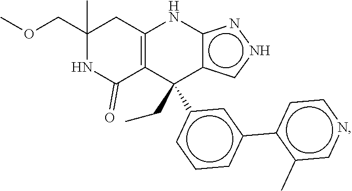 | 948127 |
| 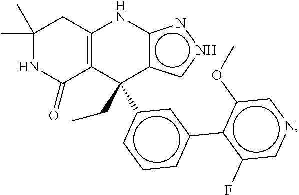 | 948145 |
| 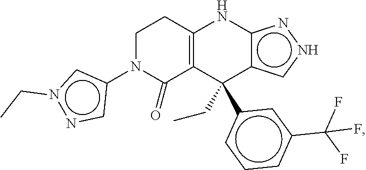 | 949868 |
| 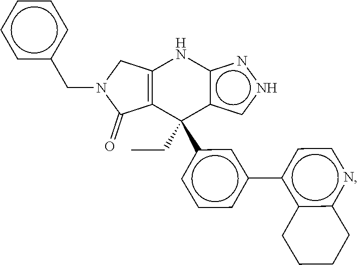 | 948552 |
| 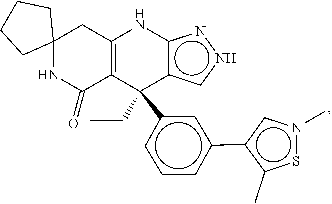 | 950255 |
| 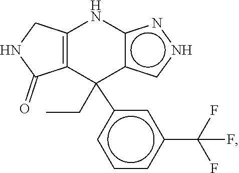 | 913156 |
| 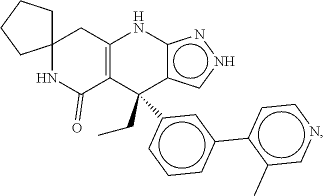 | 948021 |
| 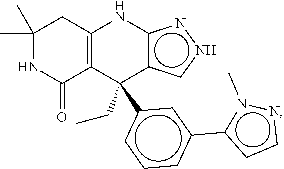 | 927390 |
| 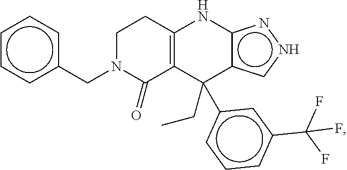 | 897749 |
| 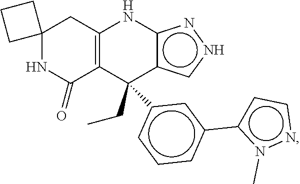 | 948522 |
| 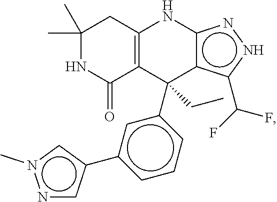 | 949911 |
| 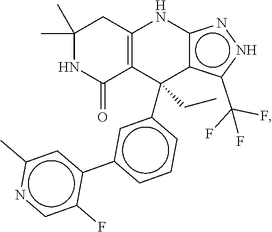 | 949644 |
| 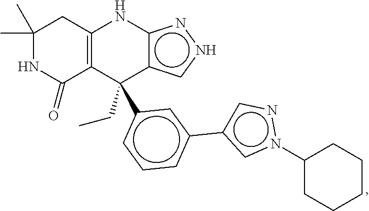 | 951707 |
| 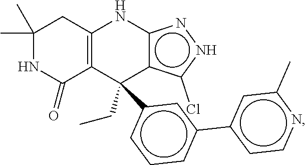 | 949421 |
| 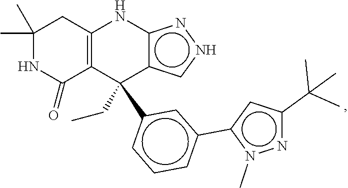 | 951843 |
| 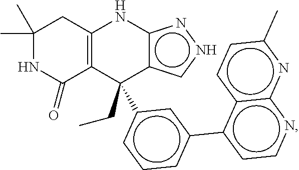 | 948151 |
| 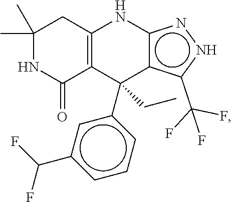 | 951946 |
| 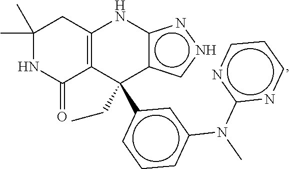 | 943965 |
| 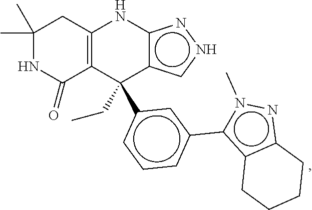 | 948652 |
| 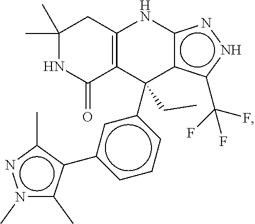 | 948417 |
| 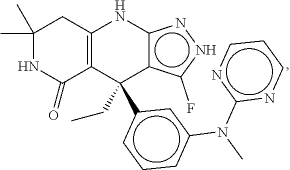 | 949647 |
| 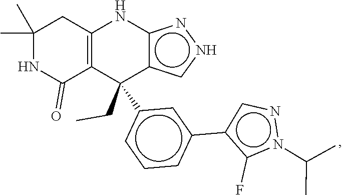 | 949267 |
| 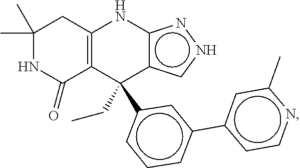 | 927326 |
| 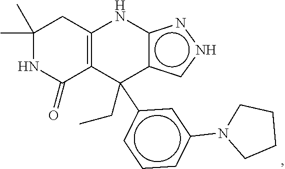 | 945723 |
| 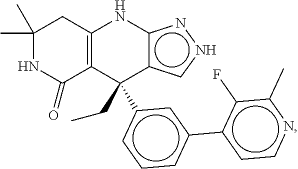 | 948311 |
| 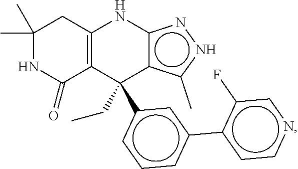 | 947805 |
| 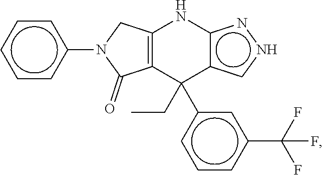 | 918313 |
| 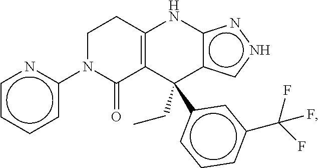 | 949311 |
| 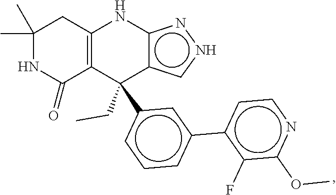 | 948531 |
| 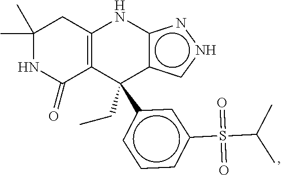 | 947823 |
| 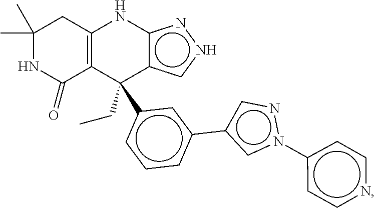 | 952912 |
| 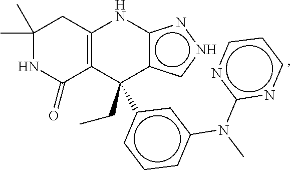 | 947825 |
| 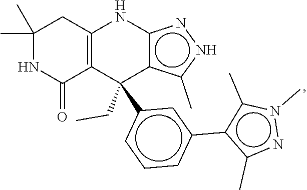 | 947573 |
| 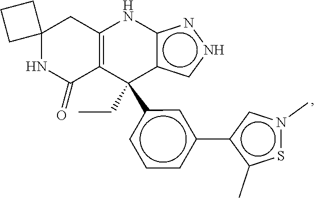 | 950056 |
| 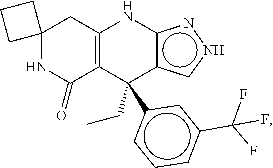 | 914344 |
| 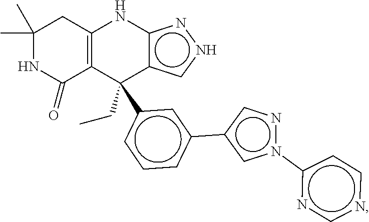 | 952016 |
| 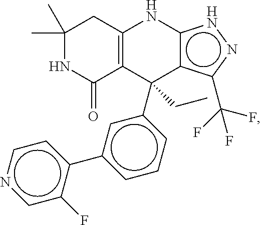 | 948770 |
| 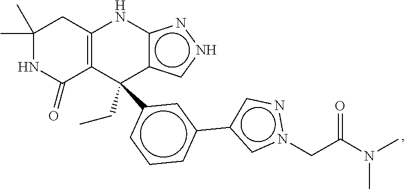 | 952208 |
| 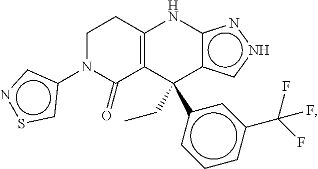 | 951742 |
| 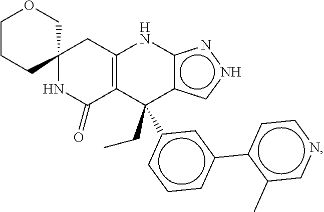 | 947912 |
| 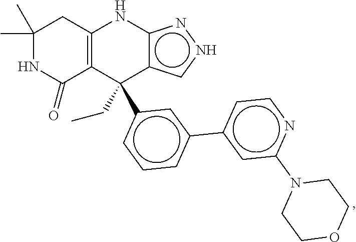 | 948825 |
| 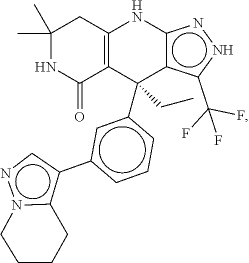 | 948772 |
| 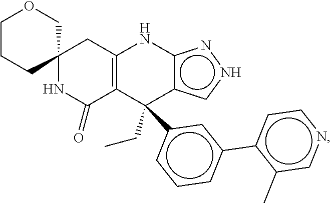 | 947909 |
| 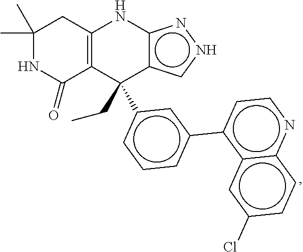 | 948530 |
| 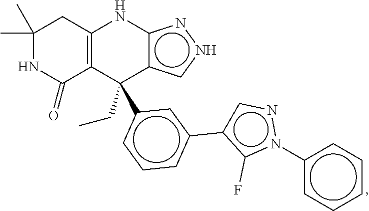 | 950821 |
| 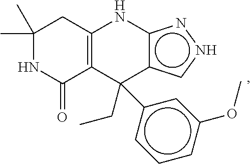 | 948296 |
| 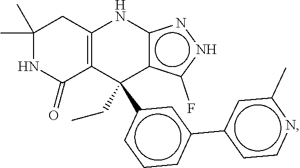 | 949001 |
| 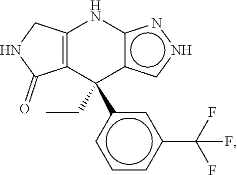 | 917158 |
| 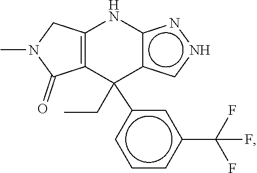 | 917294 |
| 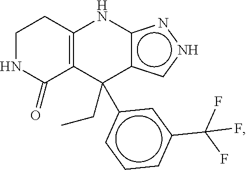 | 870263 |
| 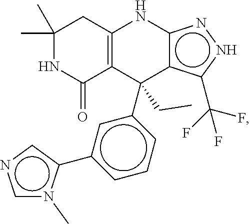 | 949003 |
| 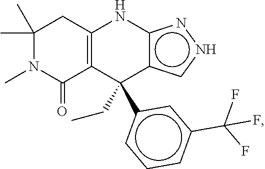 | 826791 |
| 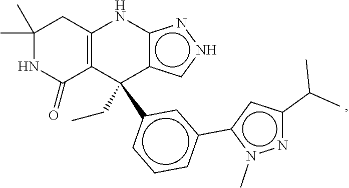 | 951842 |
| 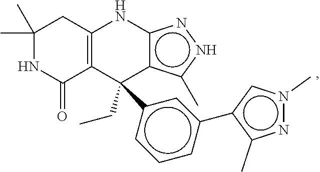 | 945996 |
| 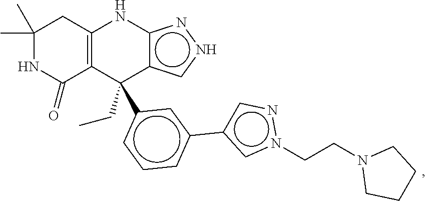 | 952022 |
| 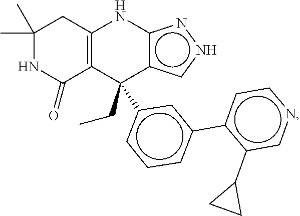 | 948642 |
| 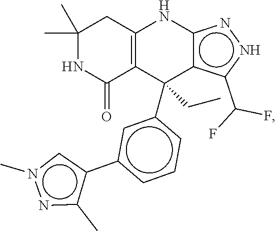 | 950490 |
| 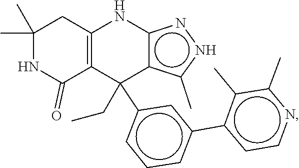 | 947399 |
| 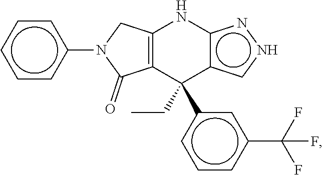 | 945377 |
| 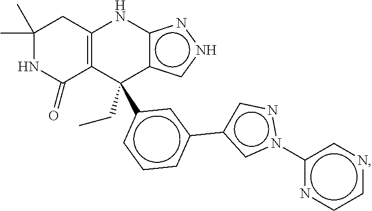 | 952207 |
| 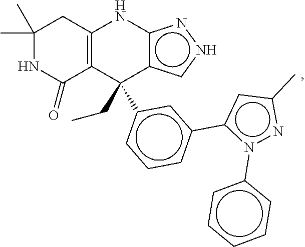 | 951845 |
| 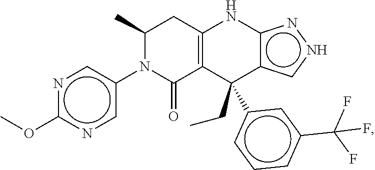 | 949354 |
| 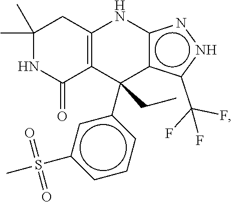 | 948422 |
| 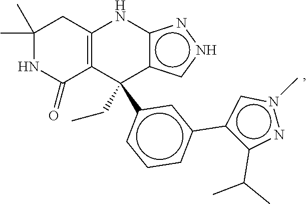 | 949111 |
| 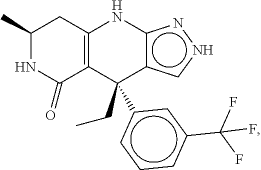 | 912097 |
| 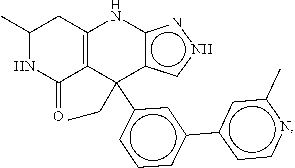 | 913357 |
| 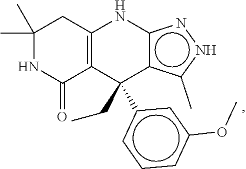 | 949337 |
| 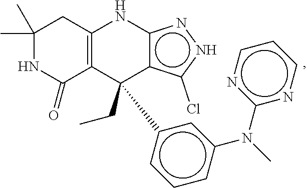 | 951618 |
| 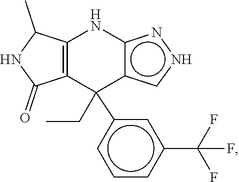 | 917295 |
| 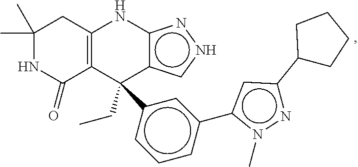 | 952023 |
| 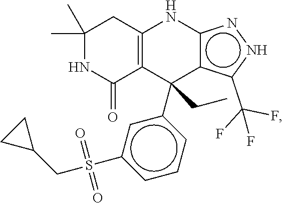 | 949186 |
| 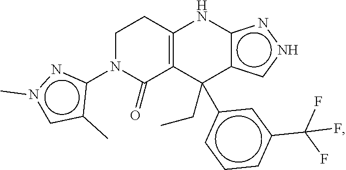 | 950486 |
| 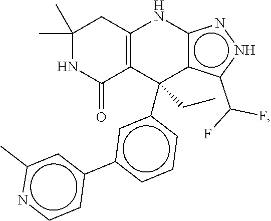 | 950162 |
| 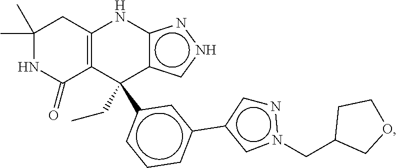 | 951836 |
| 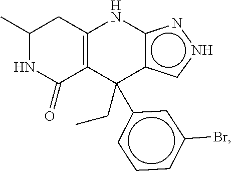 | 913225 |
| 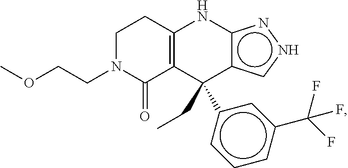 | 949599 |
| 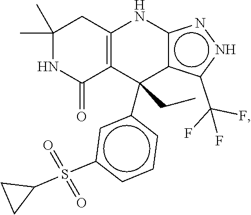 | 948776 |
| 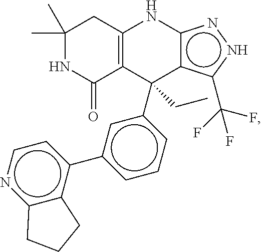 | 948769 |
| 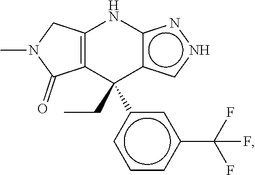 | 945367 |
| 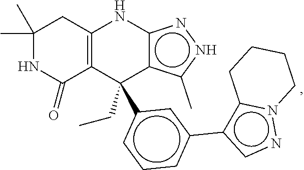 | 947809 |
| 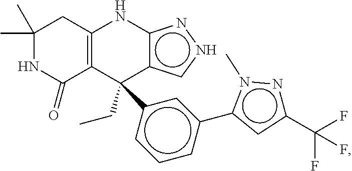 | 952018 |
| 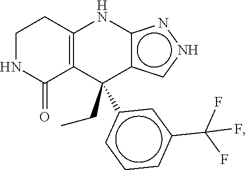 | 912622 |
| 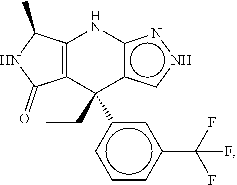 | 945379 |
| 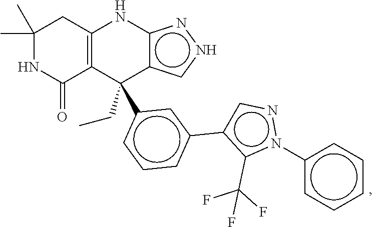 | 951837 |
| 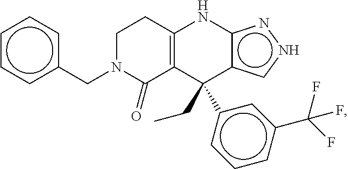 | 912005 |
| 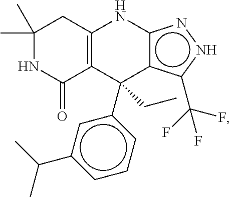 | 950493 |
| 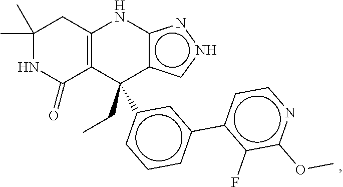 | 948520 |
| 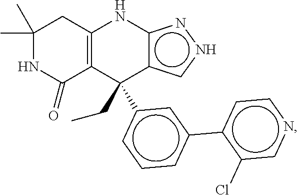 | 948020 |
| 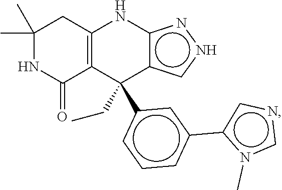 | 947820 |
| 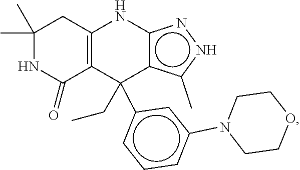 | 947564 |
| 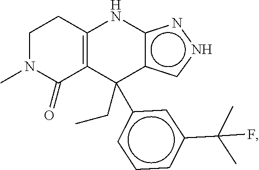 | 913178 |
| 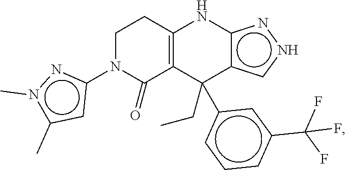 | 950608 |
| 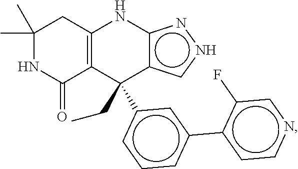 | 947874 |
| 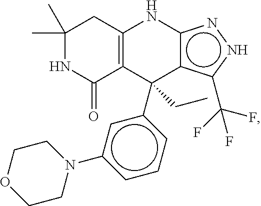 | 949000 |
| 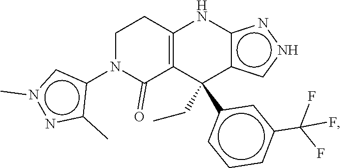 | 949642 |
| 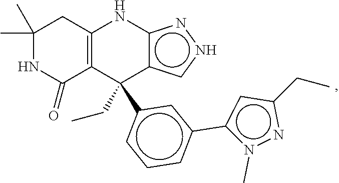 | 952561 |
| 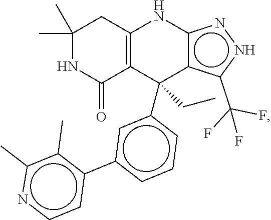 | 948181 |
| 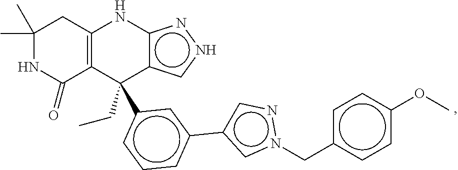 | 951838 |
| 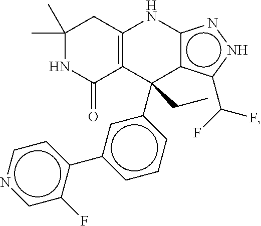 | 951599 |
| 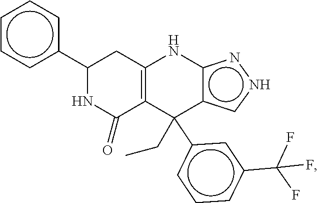 | 917284 |
| 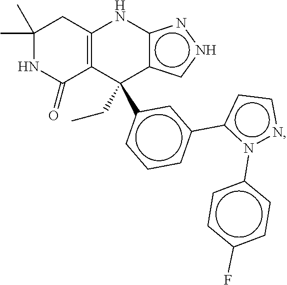 | 950822 |
| 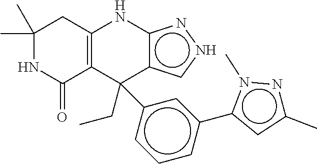 | 943371 |
| 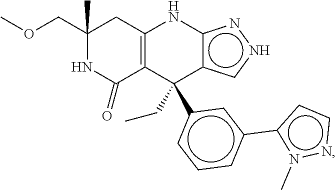 | 947905 |
| 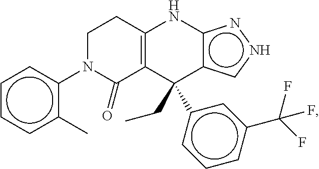 | 949128 |
| 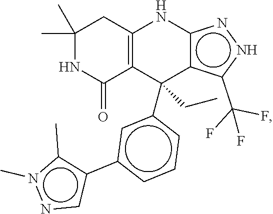 | 947807 |
| 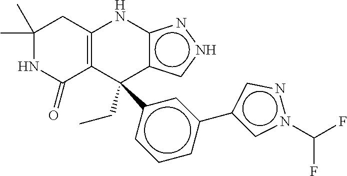 | 950254 |
| 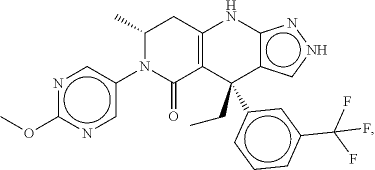 | 949353 |
| 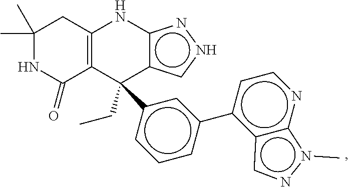 | 952913 |
| 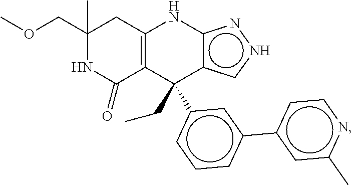 | 948521 |
| 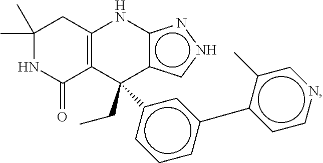 | 947576 |
| 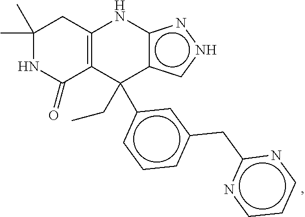 | 950151 |
| 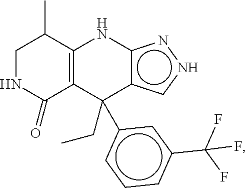 | 919318 |
| 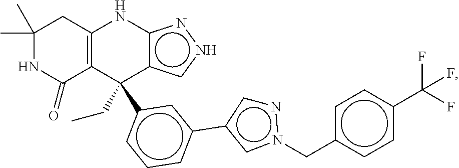 | 951849 |
| 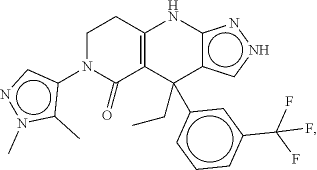 | 950612 |
| 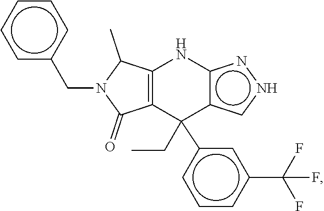 | 917162 |
| 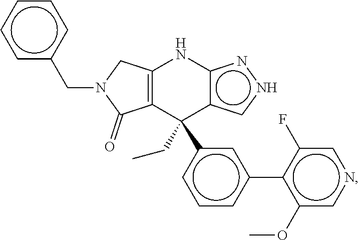 | 948550 |
| 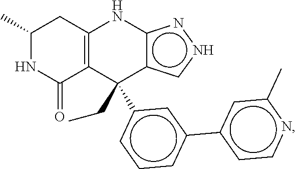 | 926212 |
| 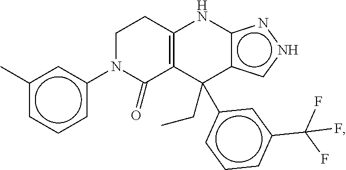 | 948052 |
| 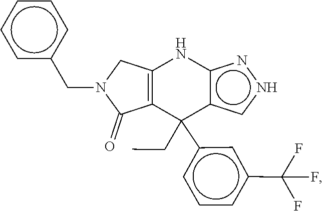 | 917159 |
| 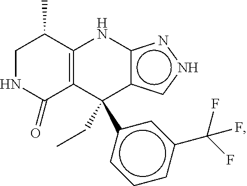 | 945382 |
| 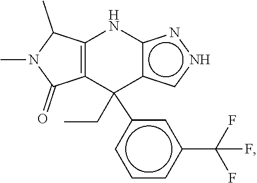 | 911957 |
| 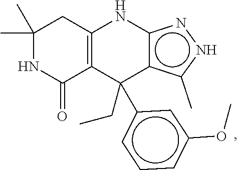 | 948763 |
| 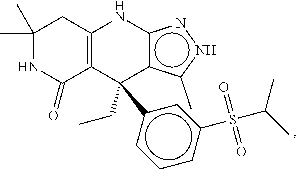 | 947870 |
| 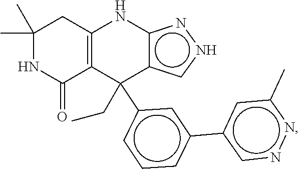 | 943496 |
| 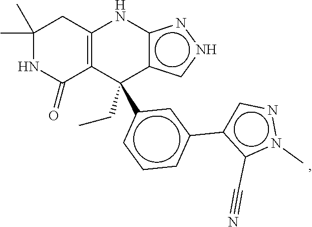 | 948827 |
| 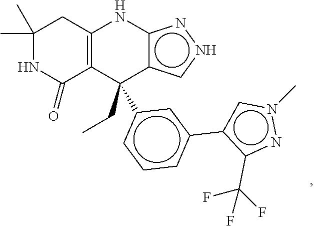 | 949266 |
| 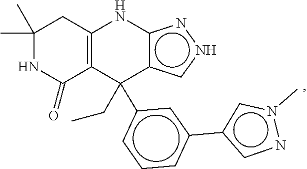 | 943912 |
| 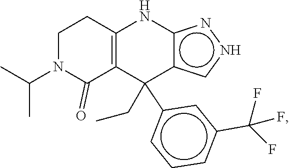 | 825755 |
| 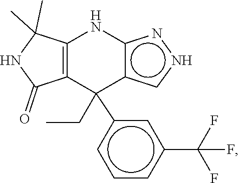 | 912104 |
| 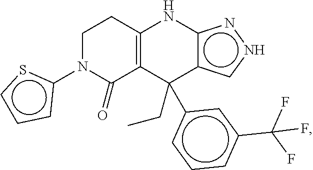 | 950614 |
| 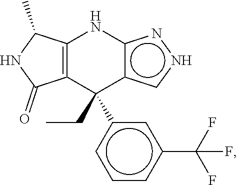 | 945378 |
| 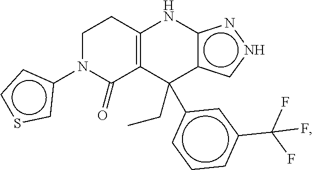 | 950776 |
| 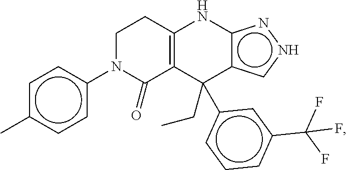 | 948241 |
| 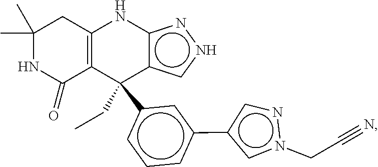 | 952210 |
| 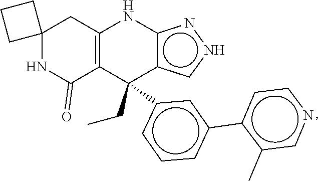 | 948523 |
| 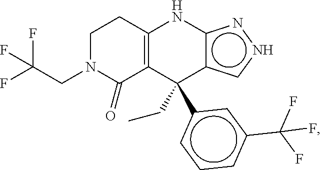 | 912206 |
| 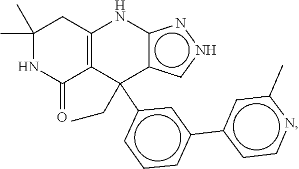 | 914930 |
| 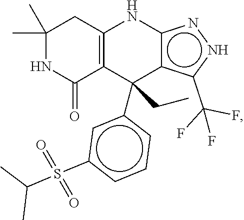 | 948423 |
| 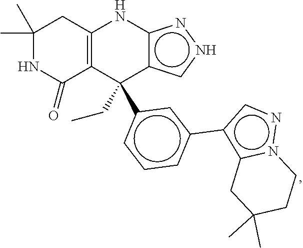 | 950564 |
| 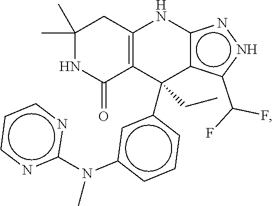 | 951736 |
| 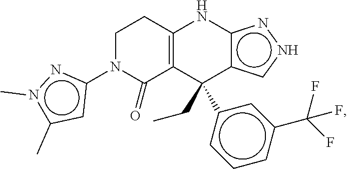 | 951939 |
| 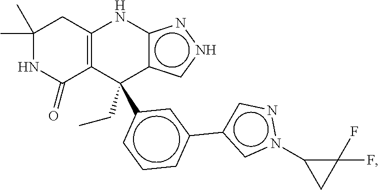 | 950060 |
| 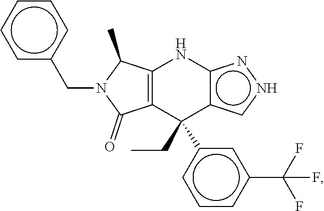 | 930331 |
| 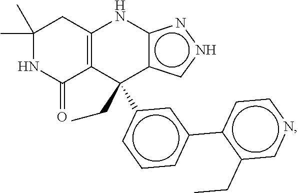 | 948639 |
| 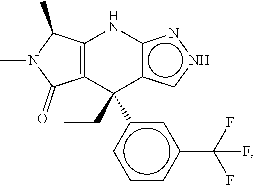 | 913473 |
| 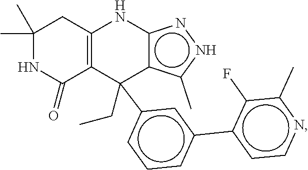 | 947563 |
| 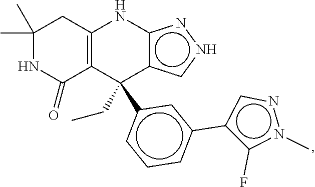 | 950562 |
| 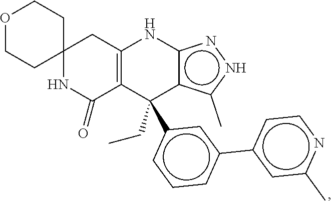 | 948513 |
| 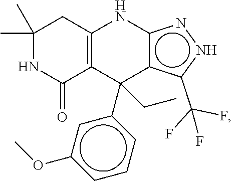 | 949748 |
| 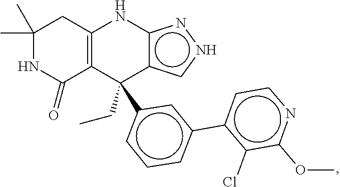 | 952754 |
| 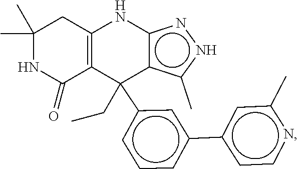 | 947798 |
| 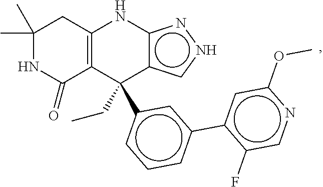 | 949109 |
| 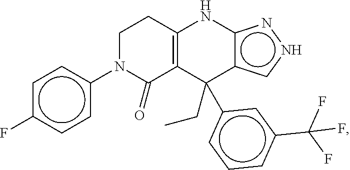 | 947748 |
| 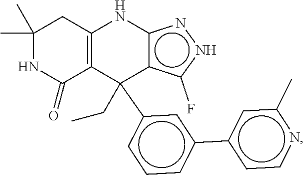 | 948299 |
| 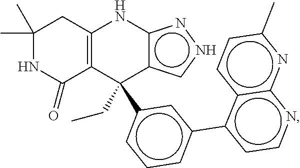 | 948143 |
| 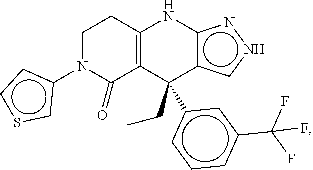 | 952630 |
| 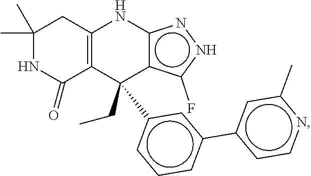 | 949012 |
| 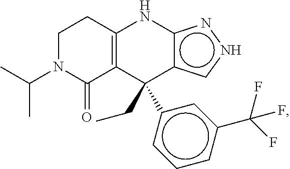 | 857336 |
| 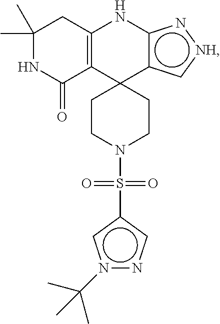 | 947835 |
| 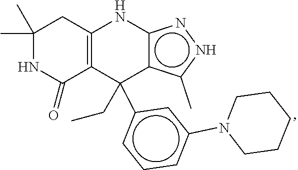 | 947674 |
| 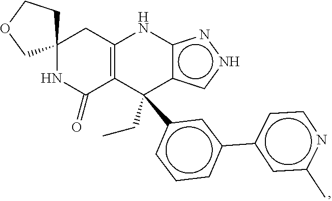 | 948149 |
| 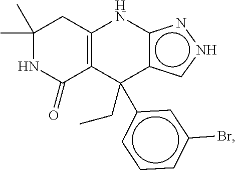 | 913439 |
| 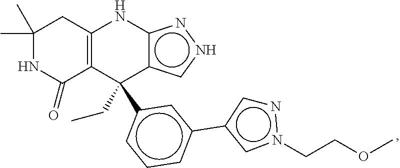 | 949105 |
| 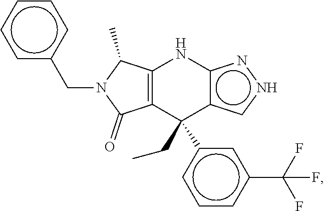 | 930330 |
| 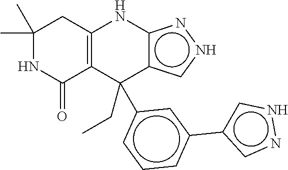 | 944022 |
| 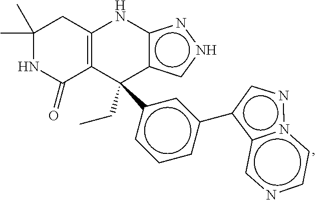 | 952565 |
| 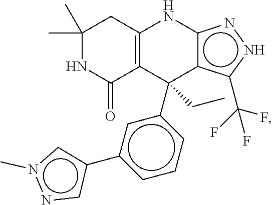 | 947871 |
| 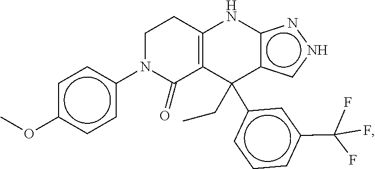 | 947332 |
| 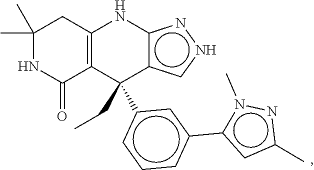 | 943966 |
| 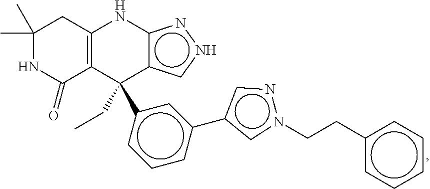 | 952017 |
| 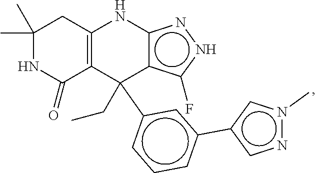 | 948300 |
| 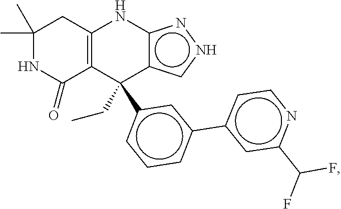 | 950561 |
| 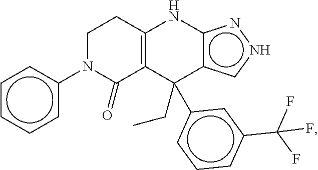 | 917211 |
| 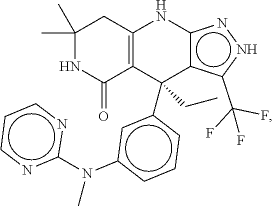 | 949180 |
| 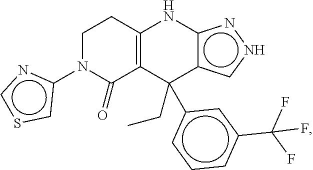 | 950777 |
| 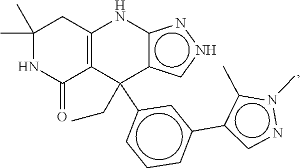 | 944021 |
| 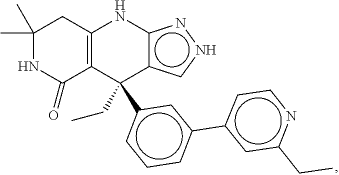 | 948370 |
| 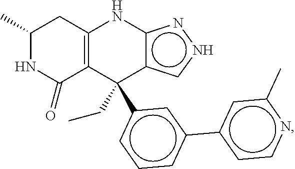 | 926300 |
| 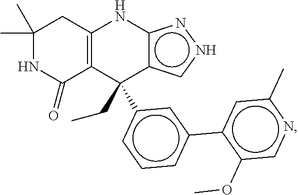 | 949522 |
| 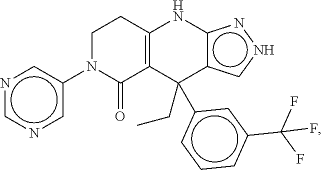 | 948238 |
| 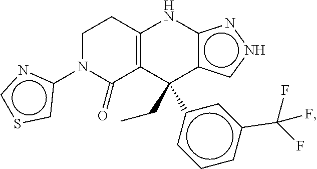 | 952629 |
| 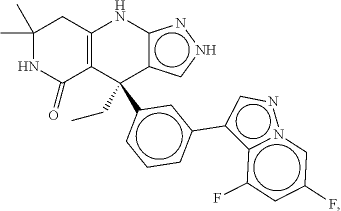 | 950565 |
| 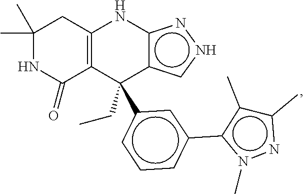 | 950062 |
| 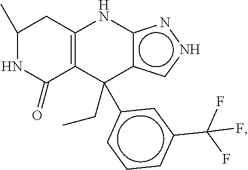 | 911956 |
| 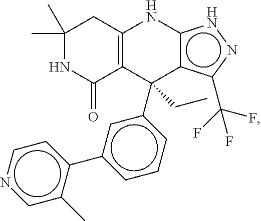 | 947812 |
| 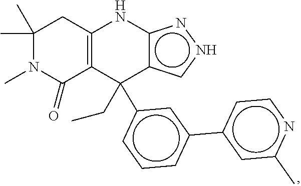 | 948302 |
| 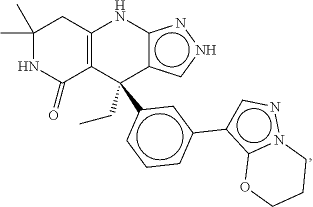 | 952019 |
| 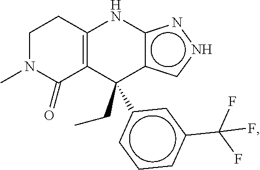 | 917282 |
| 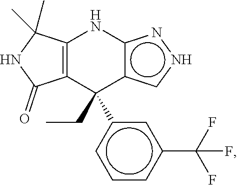 | 913451 |
| 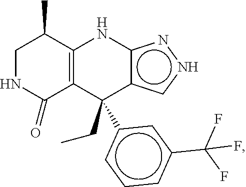 | 945383 |
| 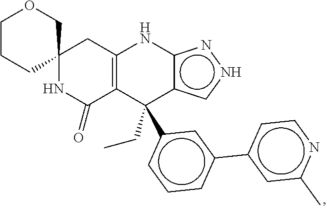 | 948152 |
| 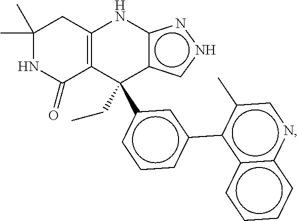 | 949398 |
| 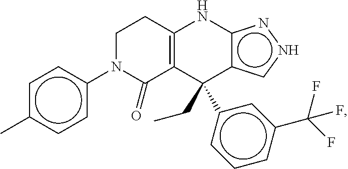 | 949118 |
| 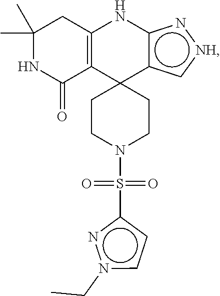 | 947836 |
| 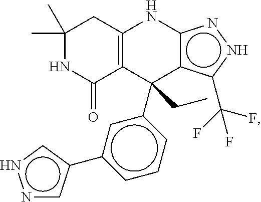 | 948312 |
| 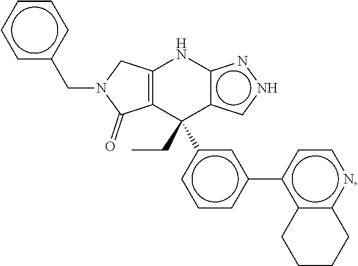 | 948551 |
| 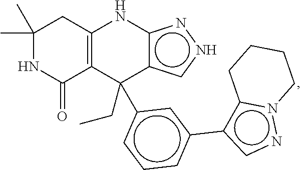 | 945603 |
| 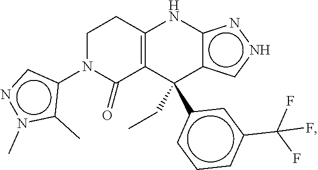 | 952257 |
| 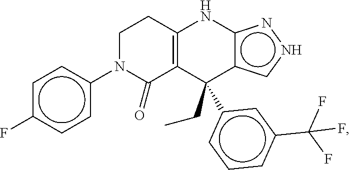 | 948224 |
| 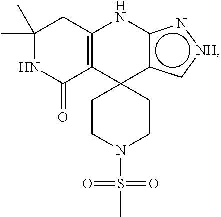 | 943959 |
| 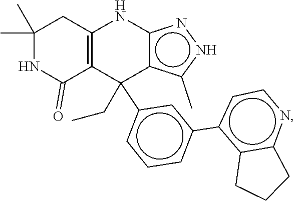 | 947675 |
| 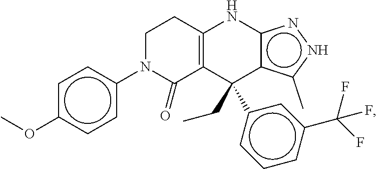 | 948226 |
| 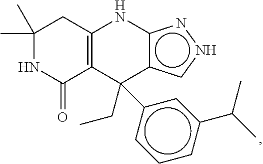 | 949326 |
| 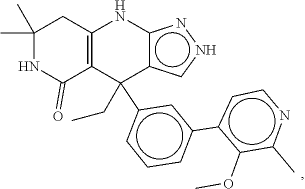 | 948794 |
| 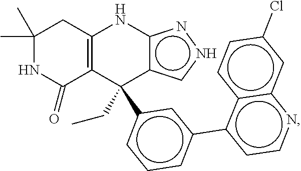 | 948368 |
| 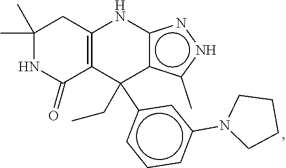 | 947673 |
| 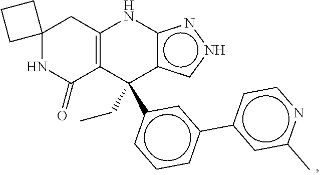 | 949520 |
| 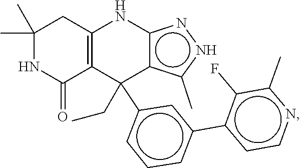 | 947402 |
| 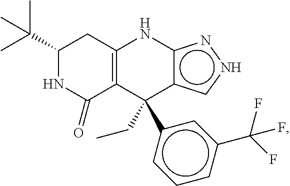 | 945922 |
| 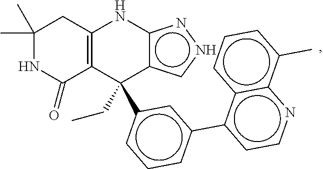 | 952909 |
| 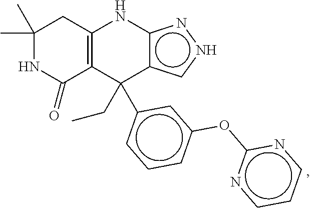 | 948764 |
| 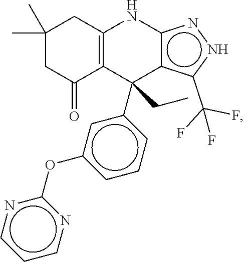 | 951604 |
| 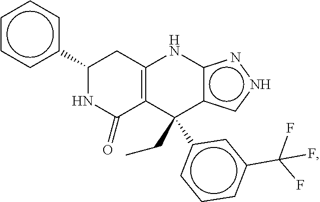 | 945361 |
| 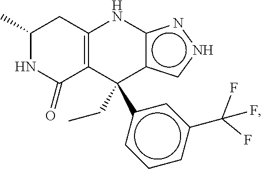 | 912098 |
| 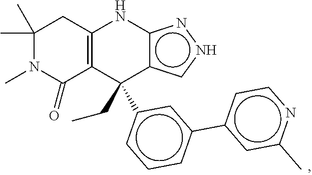 | 949004 |
| 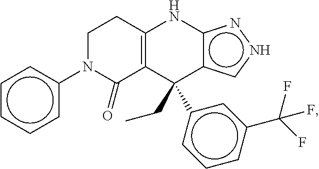 | 929474 |
| 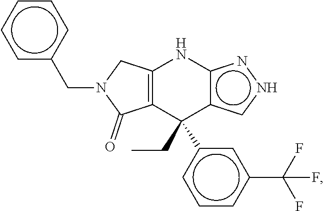 | 929388 |
| 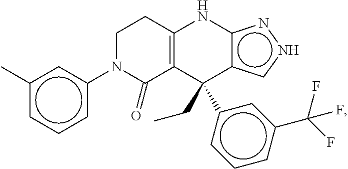 | 949040 |
| 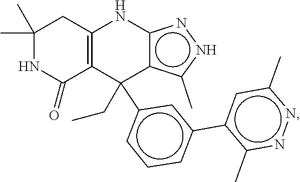 | 948766 |
| 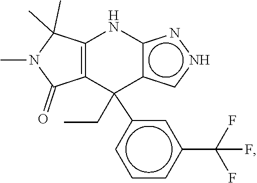 | 913198 |
| 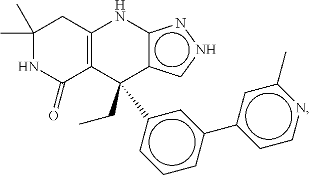 | 927325 |
| 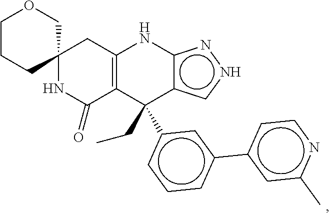 | 948144 |
| 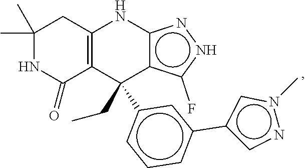 | 949011 |
| 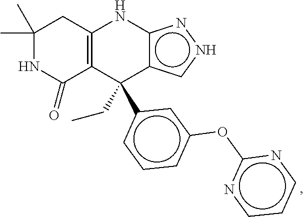 | 949330 |
| 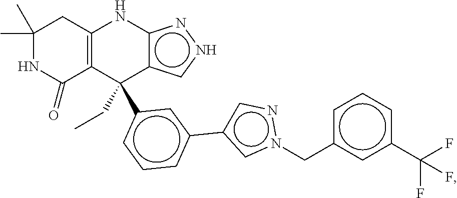 | 951848 |
| 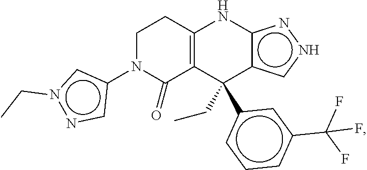 | 949867 |
| 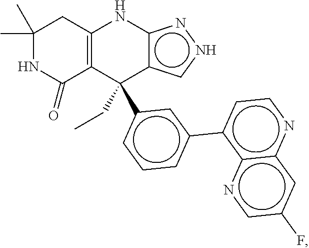 | 952758 |
| 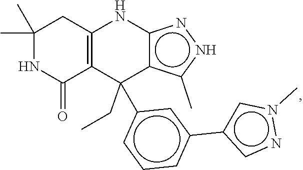 | 944027 |
| 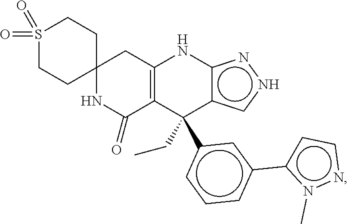 | 949394 |
| 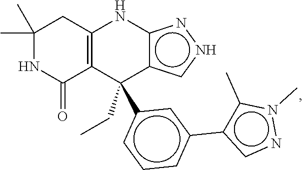 | 945608 |
| 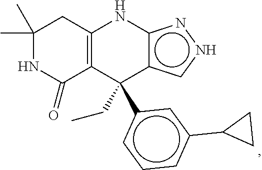 | 950828 |
| 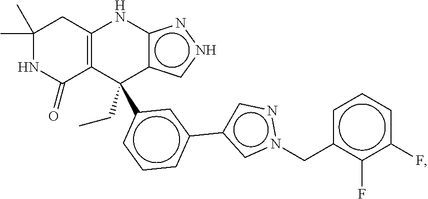 | 951840 |
| 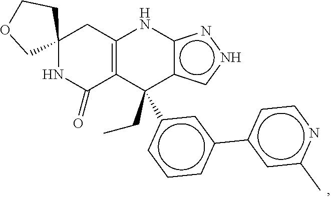 | 948140 |
| 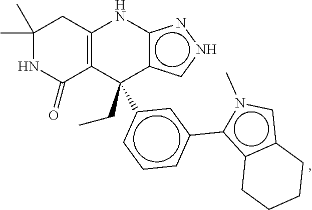 | 948655 |
| 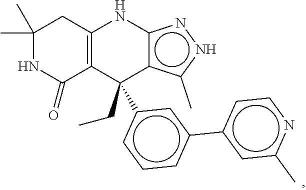 | 948420 |
| 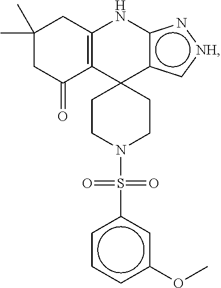 | 943962 |
| 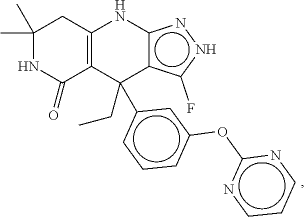 | 950615 |
| 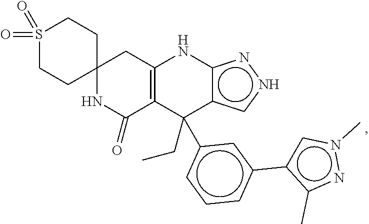 | 950813 |
| 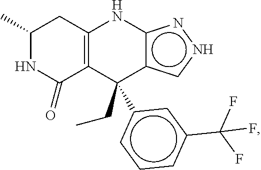 | 912096 |
| 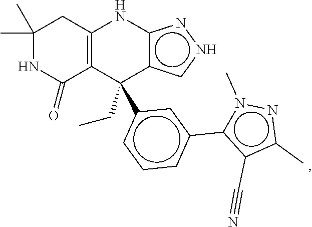 | 951844 |
| 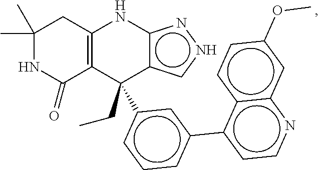 | 948661 |
| 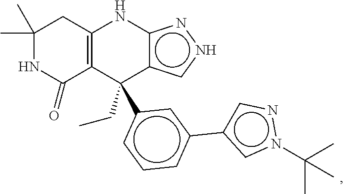 | 949112 |
| 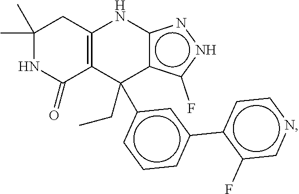 | 948999 |
| 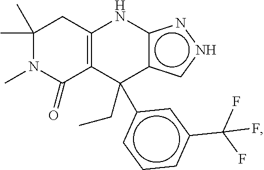 | 823130 |
| 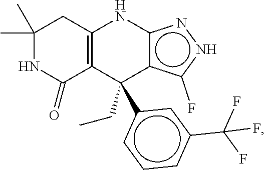 | 957518 |
| 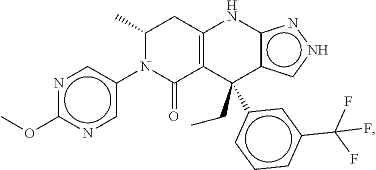 | 949351 |
| 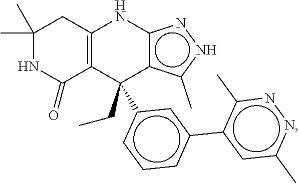 | 949333 |
| 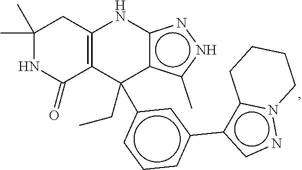 | 945993 |
| 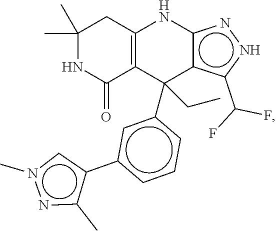 | 949170 |
| 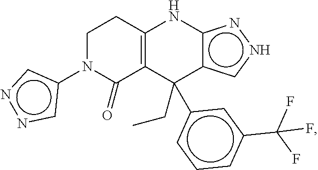 | 950479 |
| 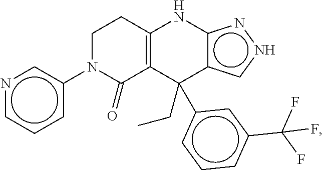 | 945637 |
| 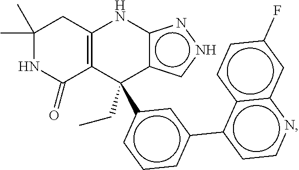 | 948528 |
| 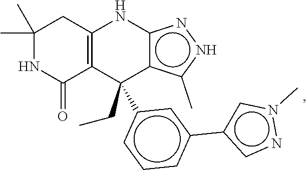 | 945614 |
| 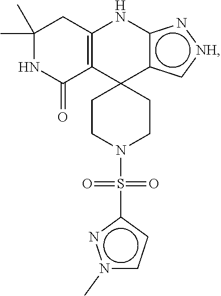 | 943961 |
| 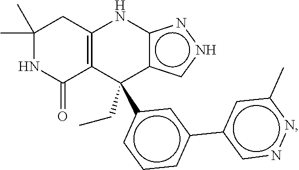 | 944074 |
| 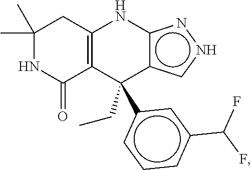 | 949006 |
| 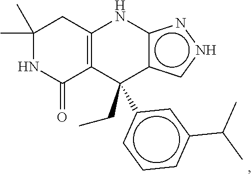 | 949761 |
| 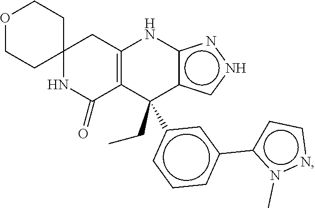 | 948516 |
| 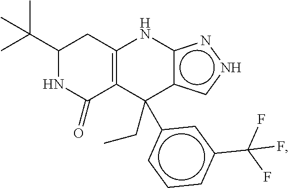 | 945635 |
| 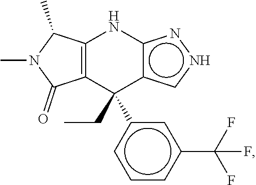 | 913454 |
| 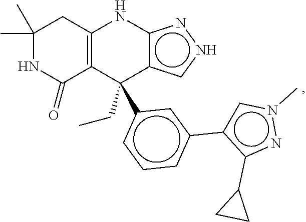 | 951706 |
| 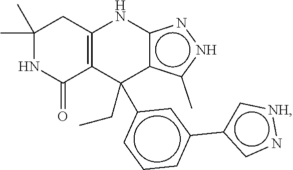 | 945459 |
| 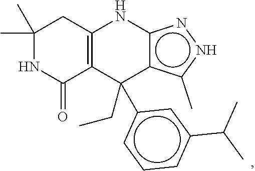 | 949173 |
| 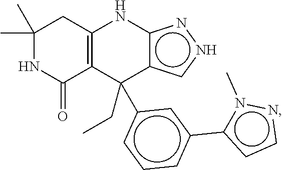 | 914971 |
| 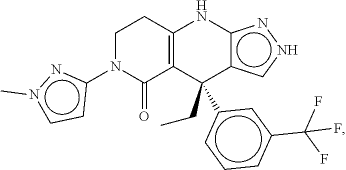 | 949835 |
| 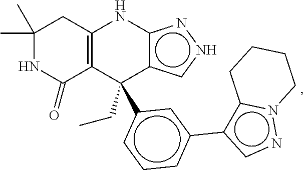 | 947410 |
| 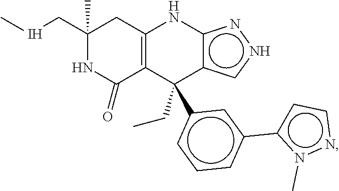 | 947906 |
| 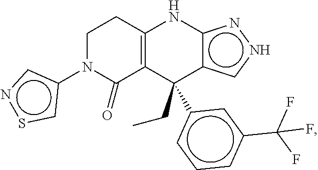 | 951733 |
| 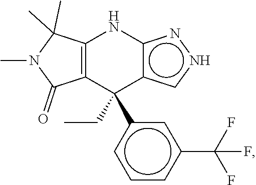 | 918252 |
| 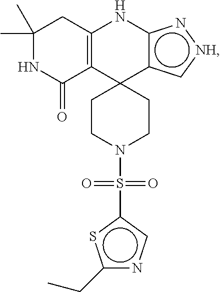 | 947837 |
| 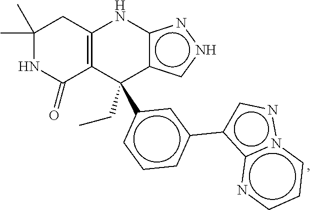 | 950830 |
| 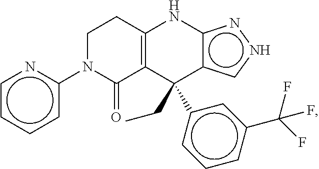 | 949310 |
| 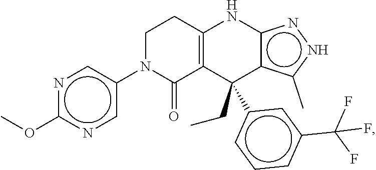 | 949126 |
| 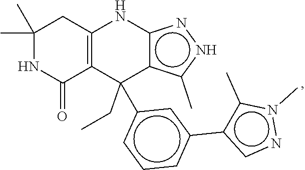 | 944028 |
| 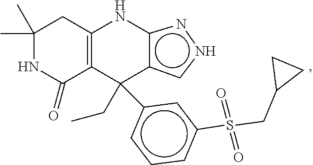 | 948177 |
| 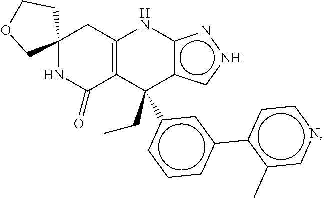 | 948640 |
| 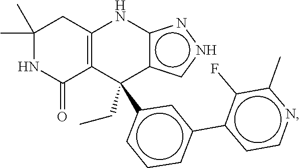 | 948306 |
| 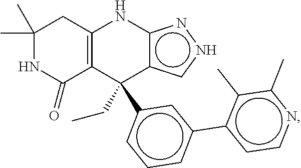 | 948184 |
| 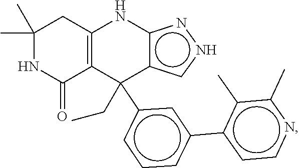 | 945994 |
| 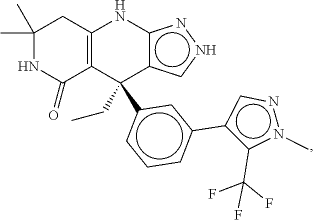 | 948815 |
| 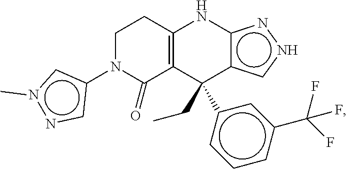 | 949270 |
| 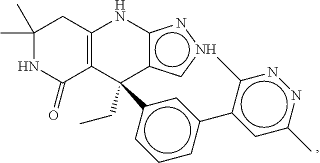 | 949331 |
| 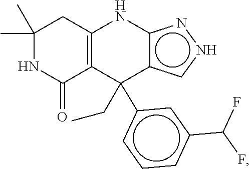 | 947864 |
| 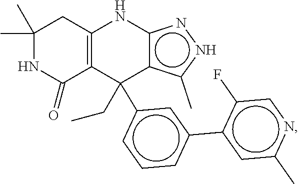 | 948761 |
| 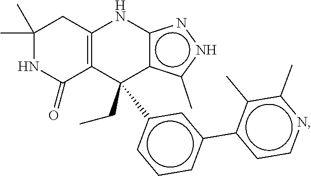 | 948187 |
| 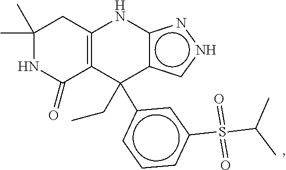 | 945464 |
| 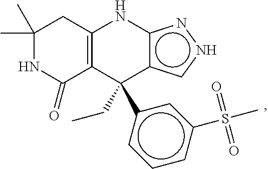 | 945604 |
| 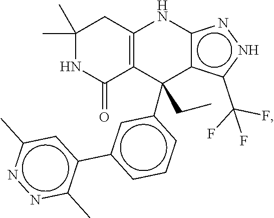 | 949910 |
| 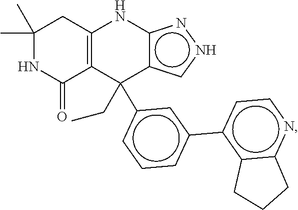 | 947676 |
| 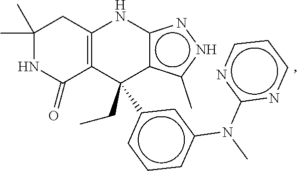 | 949182 |
| 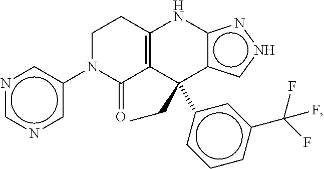 | 948379 |
| 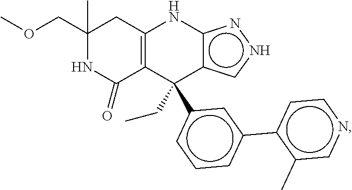 | 948147 |
| 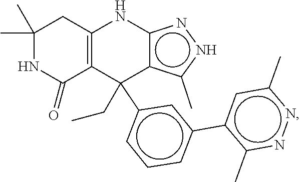 | 948765 |
| 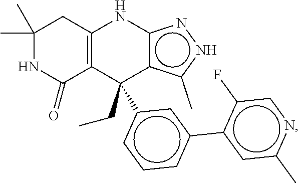 | 949338 |
| 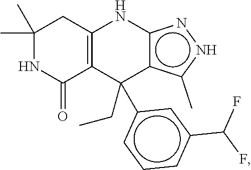 | 947865 |
| 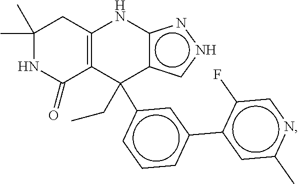 | 947401 |
| 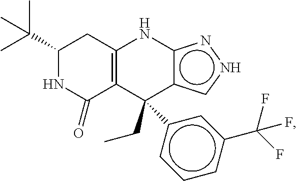 | 945920 |
| 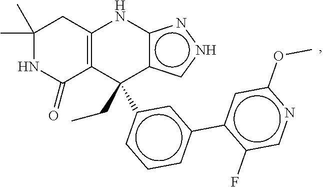 | 949108 |
| 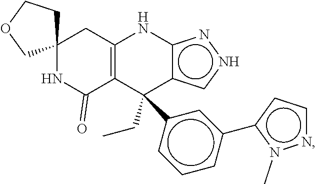 | 948015 |
| 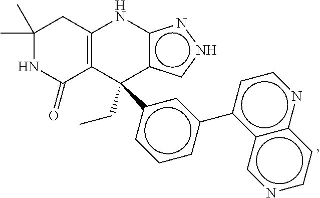 | 952910 |
| 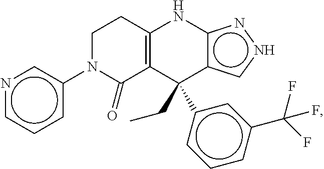 | 947432 |
| 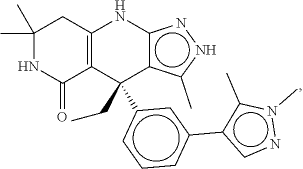 | 945731 |
| 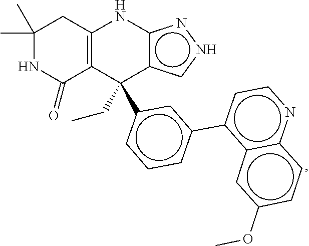 | 948527 |
| 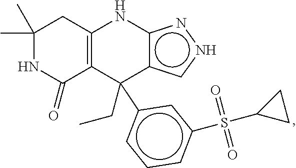 | 945465 |
| 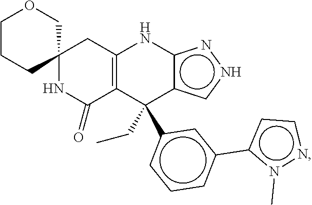 | 949115 |
| 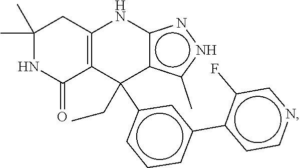 | 945722 |
| 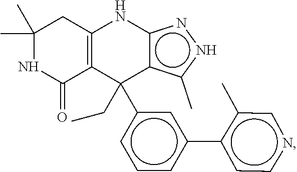 | 945599 |
| 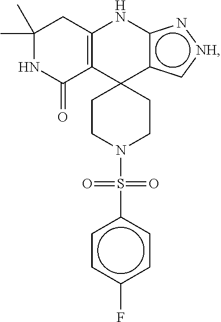 | 943963 |
| 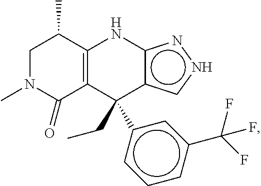 | 883979 |
| 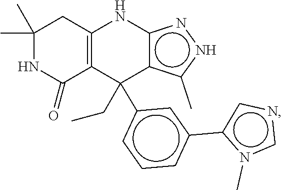 | 947565 |
| 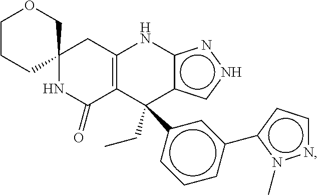 | 948529 |
| 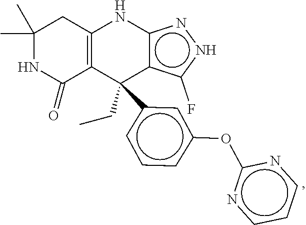 | 951949 |
| 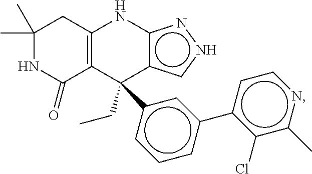 | 948361 |
| 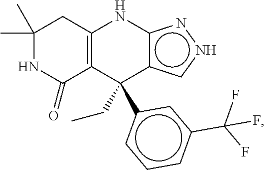 | 913006 |
| 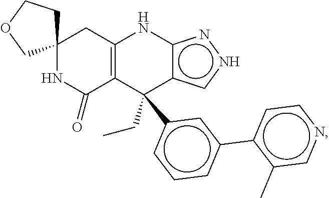 | 948644 |
| 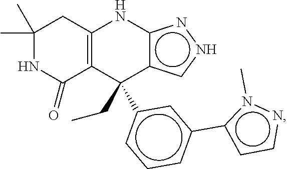 | 927389 |
| 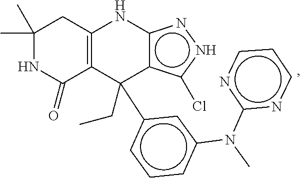 | 949417 |
| 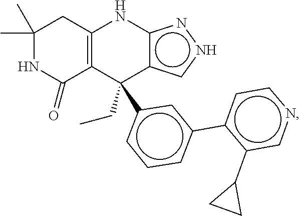 | 950059 |
| 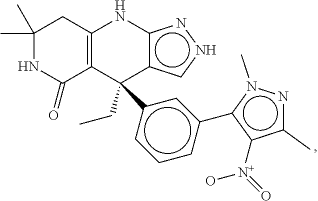 | 952209 |
| 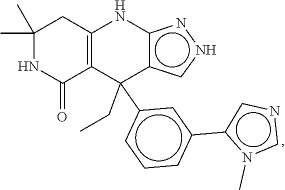 | 945597 |
| 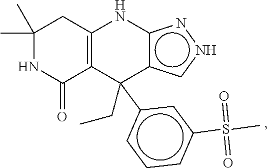 | 944020 |
| 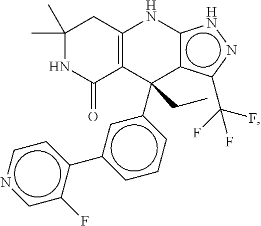 | 948777 |
| 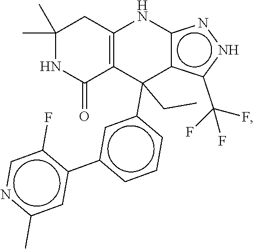 | 948762 |
| 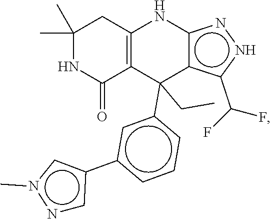 | 949171 |
| 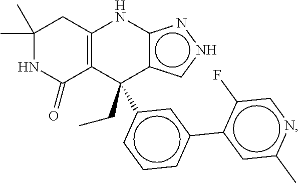 | 947804 |
| 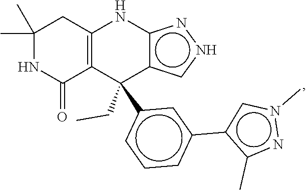 | 945470 |
| 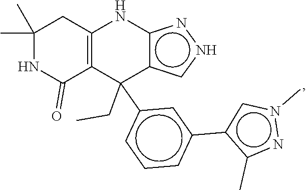 | 944030 |
| 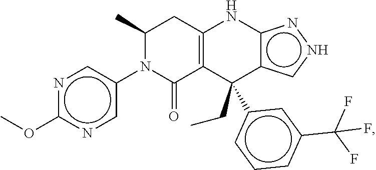 | 949352 |
| 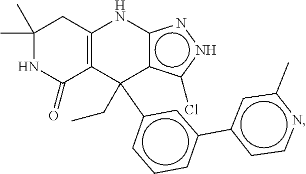 | 948998 |
| 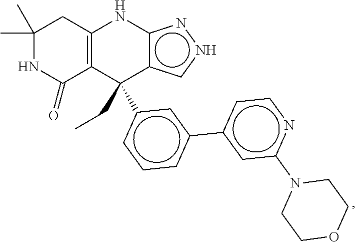 | 948653 |
| 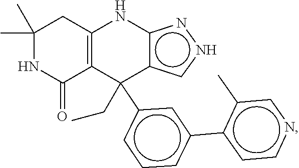 | 945461 |
| 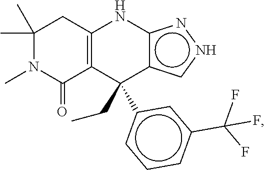 | 826790 |
| 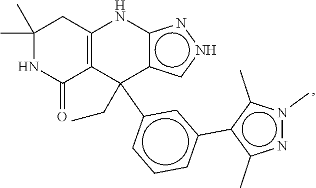 | 945462 |
| 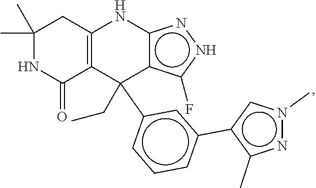 | 948301 |
| 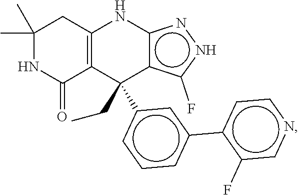 | 949651 |
| 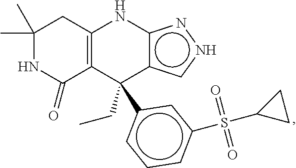 | 945999 |
| 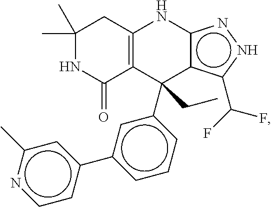 | 950163 |
| 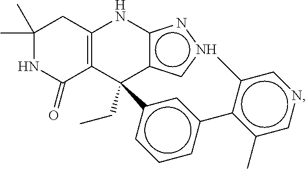 | 948812 |
| 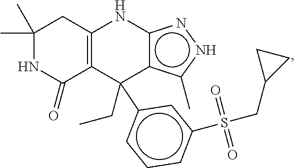 | 948178 |
| 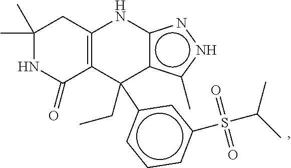 | 947396 |
| 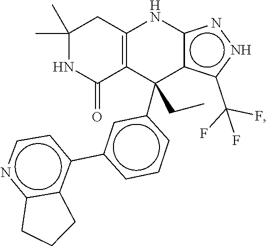 | 948778 |
| 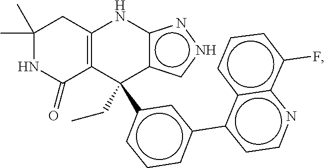 | 948828 |
| 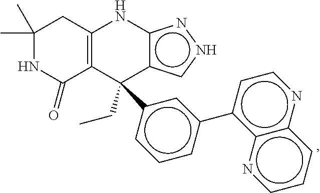 | 952560 |
| 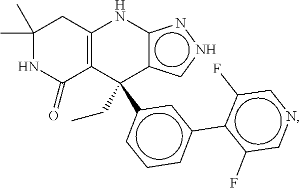 | 948525 |
| 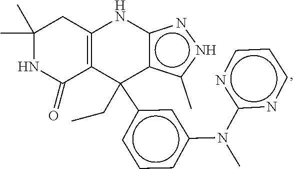 | 948175 |
| 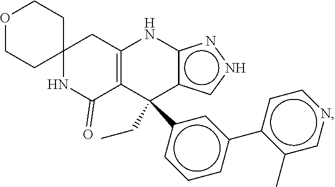 | 948359 |
| 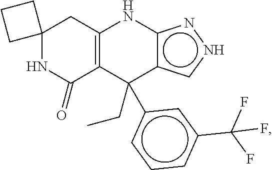 | 902202 |
| 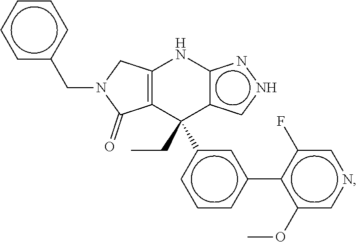 | 948549 |
| 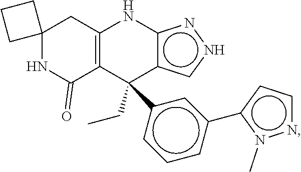 | 948358 |
| 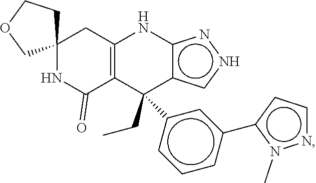 | 948017 |
| 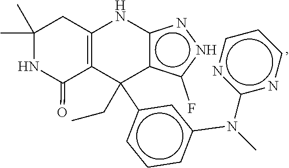 | 949172 |
| 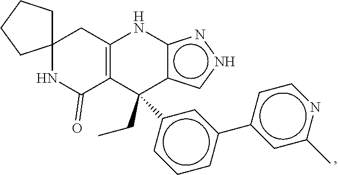 | 948660 |
| 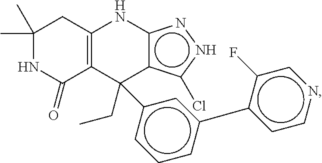 | 949324 |
| 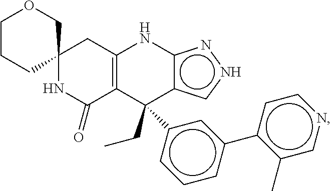 | 947911 |
| 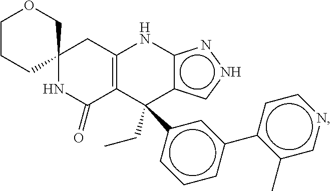 | 947910 |
| 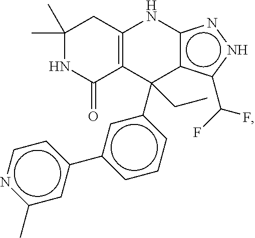 | 949169 |
| 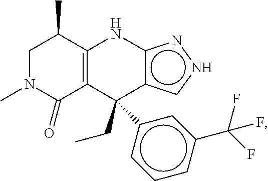 | 883980 |
| 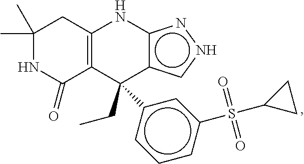 | 947803 |
| 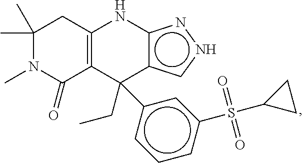 | 948303 |
| 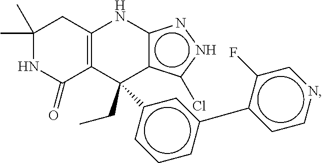 | 949757 |
| 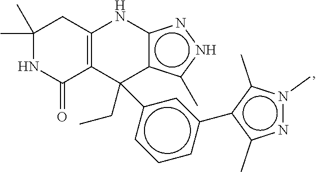 | 945598 |
| 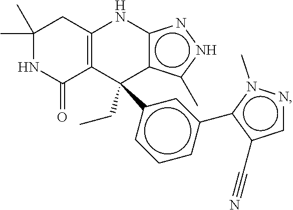 | 952410 |
| 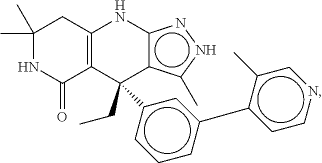 | 947414 |
| 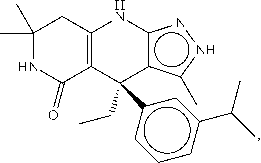 | 949649 |
| 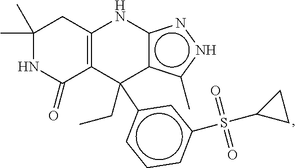 | 947562 |
| 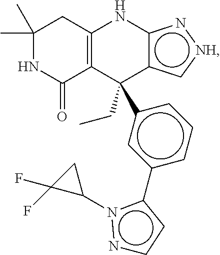 | 950824 |
| 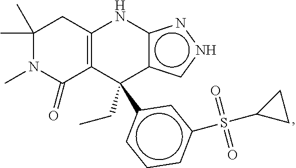 | 949177 |
| 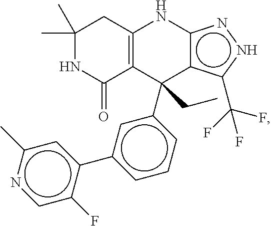 | 949650 |
| 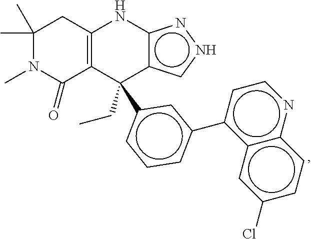 | 948517 |
| 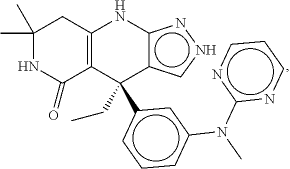 | 947826 |
| 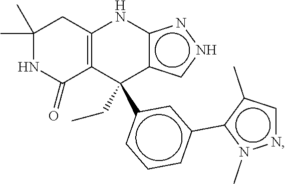 | 952021 |
| 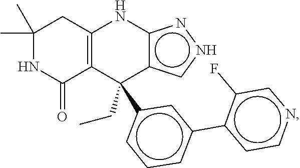 | 947869 |
| 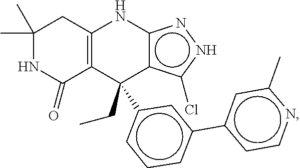 | 949422 |
| 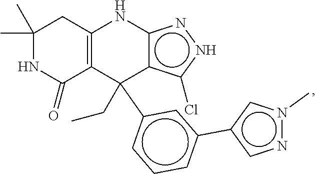 | 948767 |
| 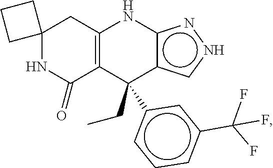 | 914308 |
| 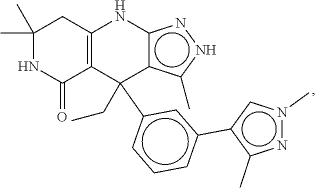 | 945460 |
| 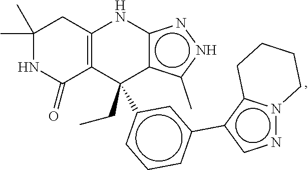 | 947817 |
| 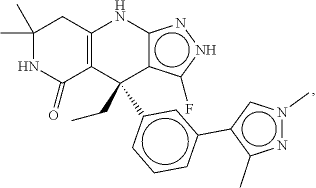 | 949185 |
| 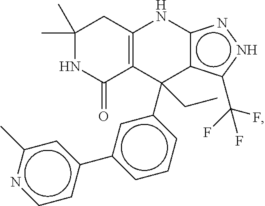 | 947858 |
| 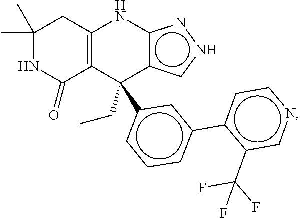 | 952025 |
| 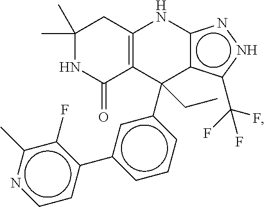 | 948297 |
| 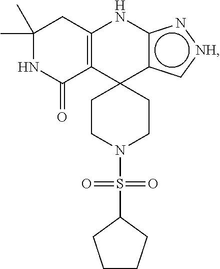 | 943960 |
| 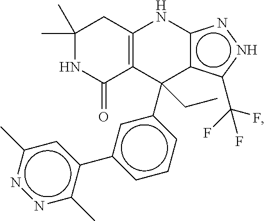 | 949174 |
| 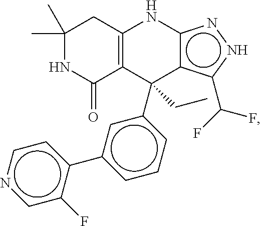 | 951612 |
| 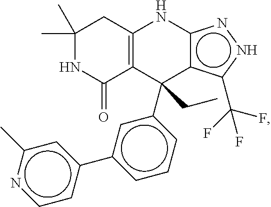 | 948774 |
| 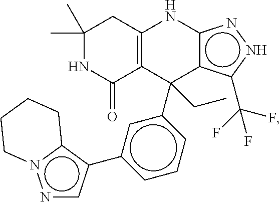 | 947801 |
| 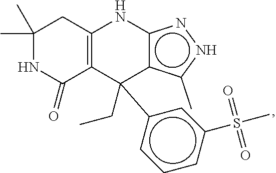 | 947395 |
| 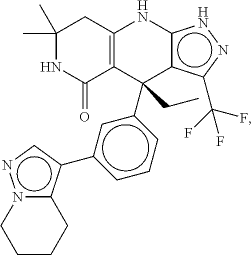 | 948775 |
| 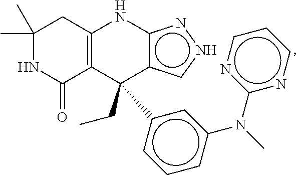 | 943964 |
| 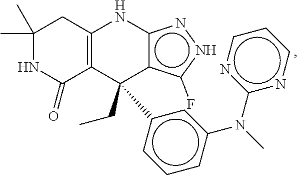 | 949648 |
| 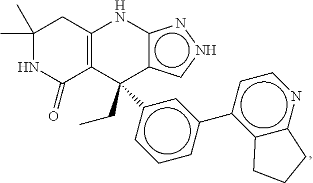 | 948313 |
| 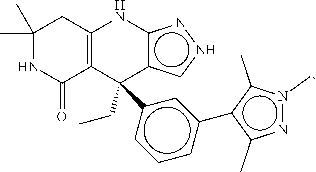 | 947408 |
| 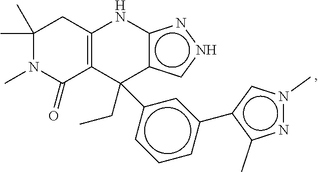 | 948304 |
| 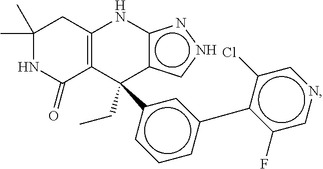 | 948371 |
| 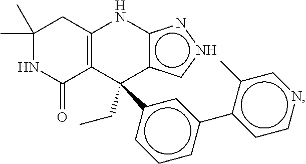 | 947574 |
| 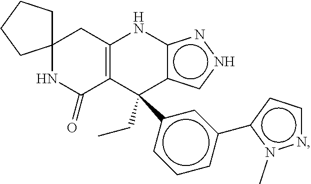 | 948018 |
| 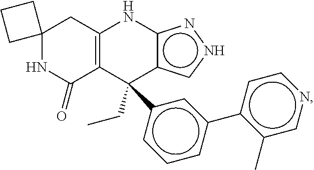 | 948512 |
| 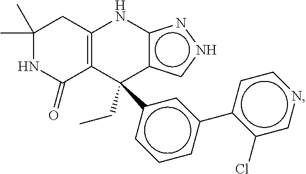 | 948142 |
| 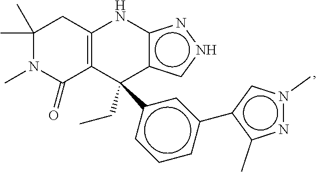 | 949005 |
| 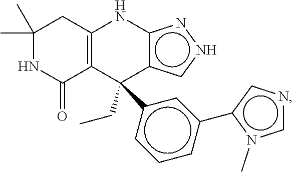 | 947806 |
| 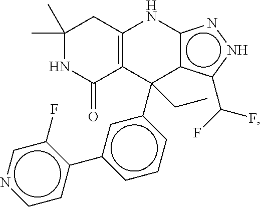 | 950320 |
| 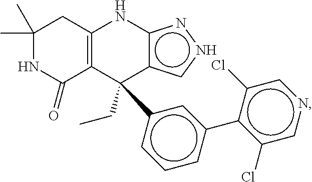 | 948515 |
| 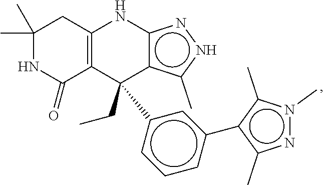 | 947575 |
| 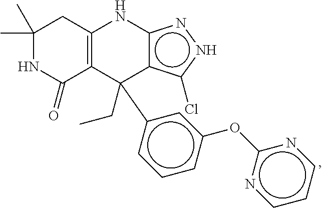 | 950318 |
| 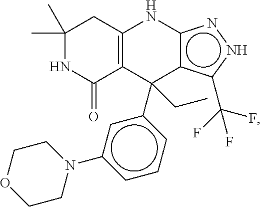 | 947863 |
| 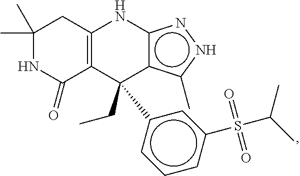 | 947876 |
| 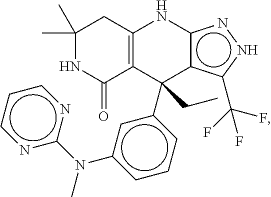 | 949181 |
| 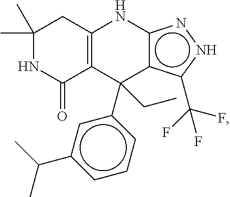 | 949747 |
| 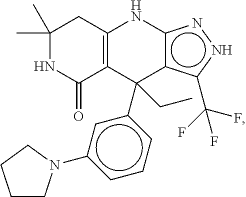 | 947860 |
| 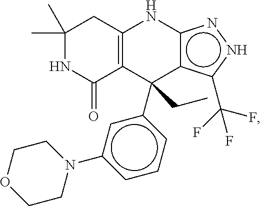 | 949013 |
| 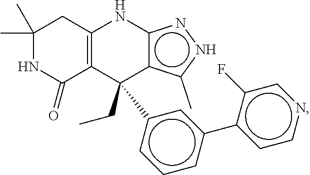 | 947821 |
| 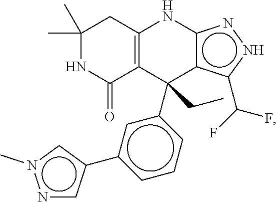 | 949919 |
| 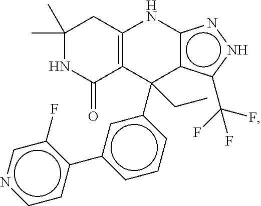 | 947866 |
| 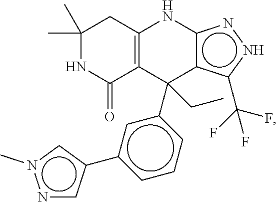 | 947405 |
| 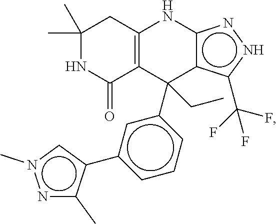 | 947567 |
| 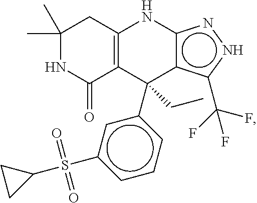 | 948771 |
| 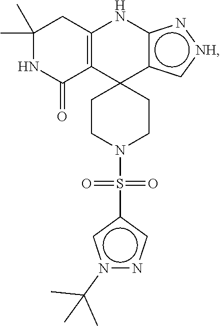 | 947835 |
| 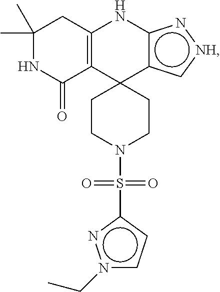 | 947836 |
| 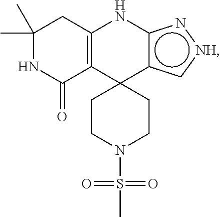 | 943959 |
| 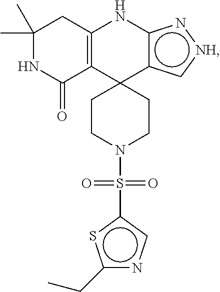 | 947837 |
| 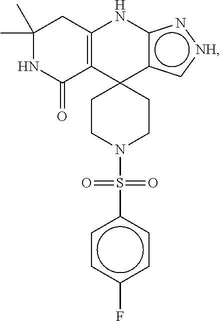 | 943963 |
| 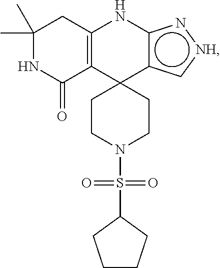 | 943960 |

In some embodiments, Formula I-A or I-B is any one of the formulae shown in Table 1A:

TABLE 1A

| | |
|---|---|
| 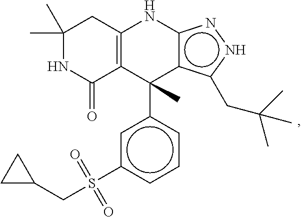 | 108005 |
| 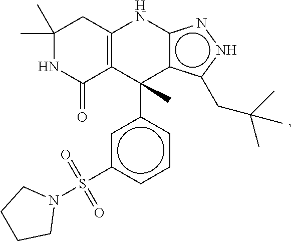 | 108791 |
| 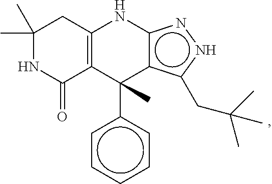 | 114531 |
| 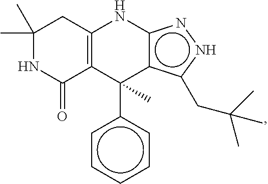 | 114532 |

or

| | |
|---|---|
| 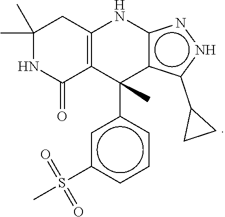 | 114679 |

In some embodiments, Formula I-A or I-B is any one of the formulae shown in Table 2:

TABLE 2

| | |
|---|---|
| 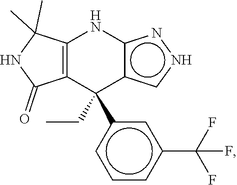 | 913452 |
| 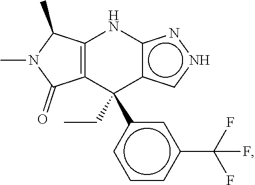 | 913495 |
| 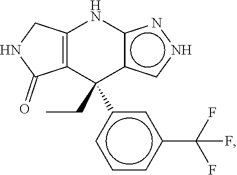 | 917157 |
| 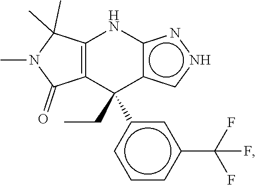 | 918251 |
| 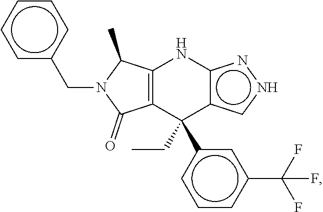 | 930333 |
| 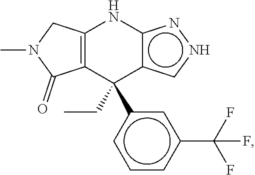 | 945368 |
| 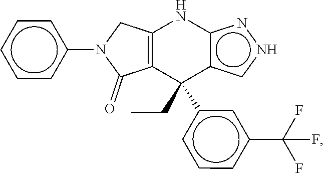 | 945376 |
| 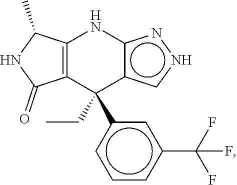 | 945380 |
| 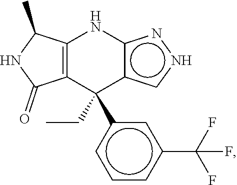 | 945381 |
| 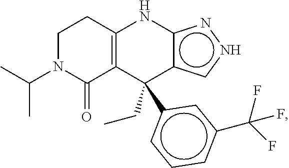 | 857362 |
| 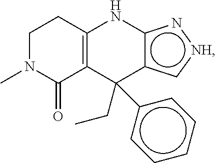 | 883867 |
| 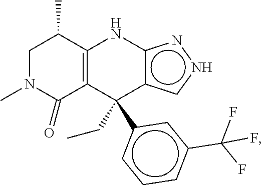 | 883981 |
| 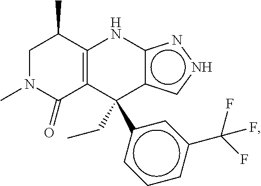 | 883982 |
| 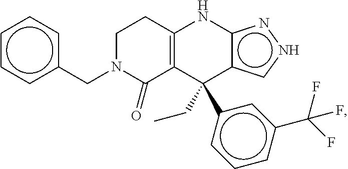 | 912006 |
| 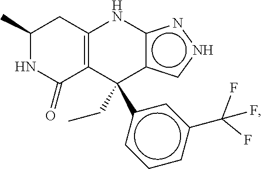 | 912099 |
| 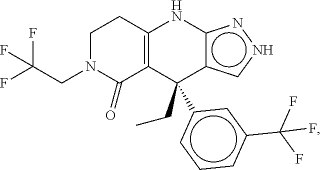 | 912205 |
| 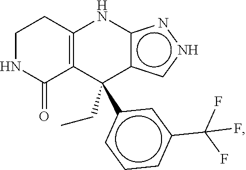 | 912668 |
| 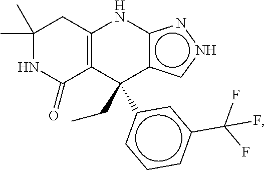 | 913004 |
| 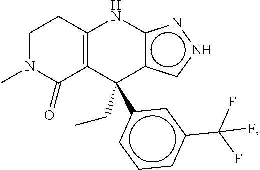 | 917283 |
| 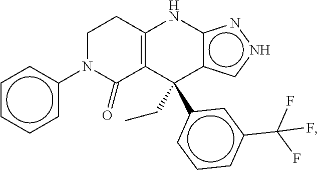 | 929475 |
| 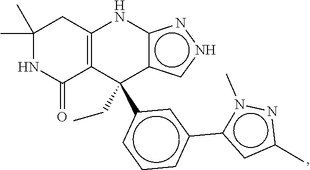 | 943967 |
| 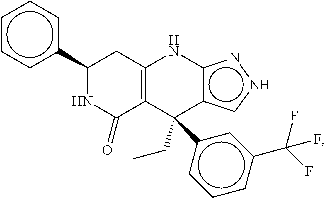 | 945364 |
| 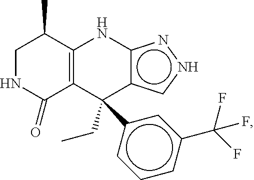 | 945385 |
| 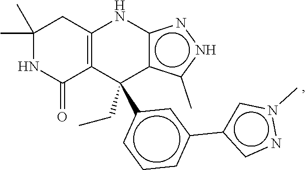 | 945611 |
| 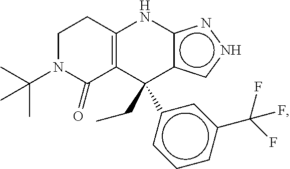 | 945633 |
| 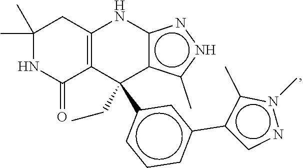 | 945727 |
| 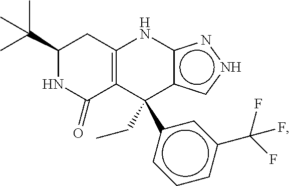 | 945923 |
| 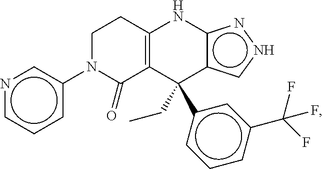 | 947433 |
| 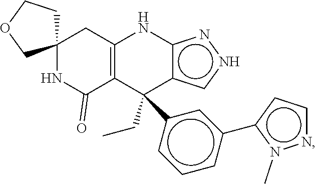 | 948016 |
| 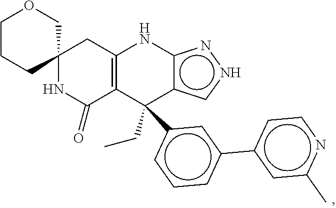 | 948153 |
| 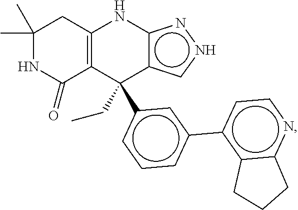 | 948308 |
| 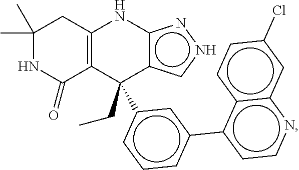 | 948362 |
| 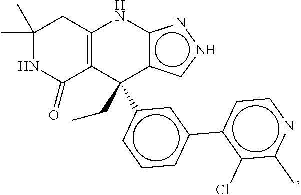 | 948369 |
| 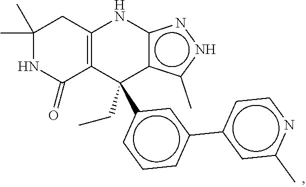 | 948414 |
| 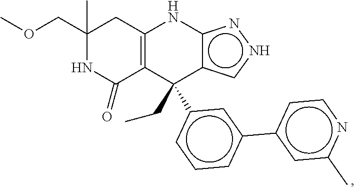 | 948493 |
| 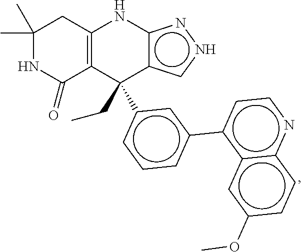 | 948518 |
| 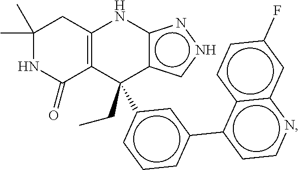 | 948519 |
| 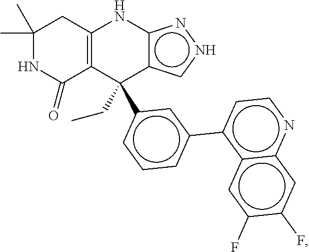 | 948638 |
| 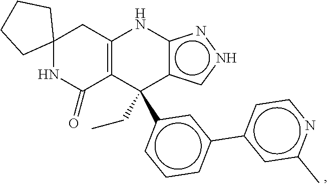 | 948641 |
| 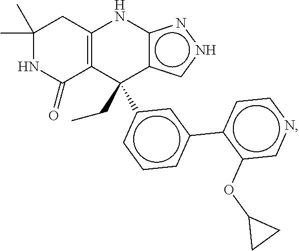 | 948643 |
| 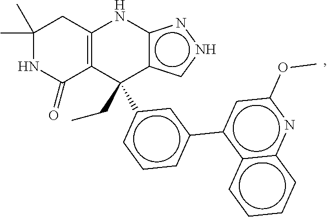 | 948647 |
| 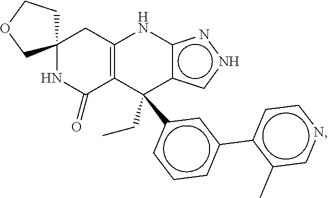 | 948648 |
| 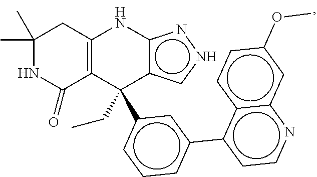 | 948649 |
| 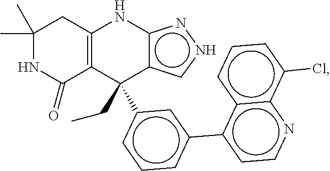 | 948650 |
| 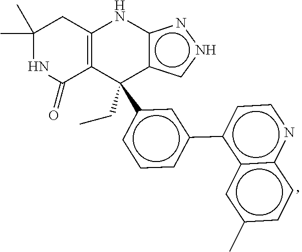 | 948651 |
| 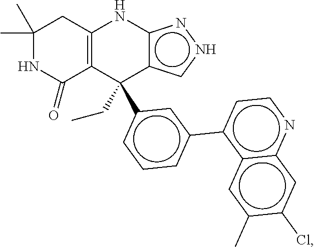 | 948654 |
| 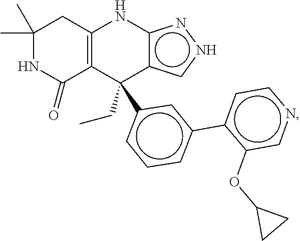 | 948656 |
| 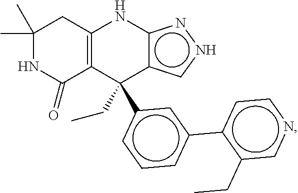 | 948657 |
| 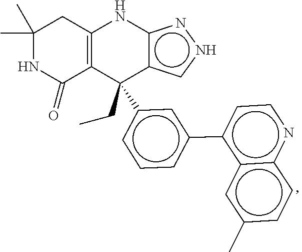 | 948658 |
| 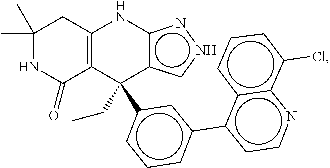 | 948659 |
| 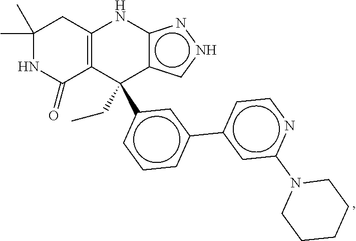 | 948663 |
| 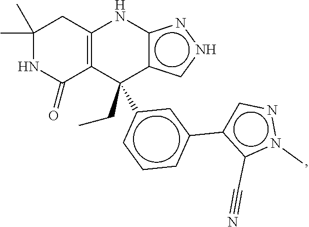 | 948814 |
| 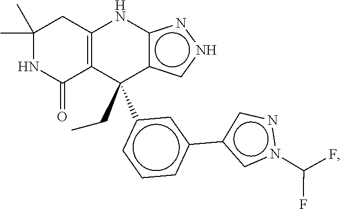 | 948816 |
| 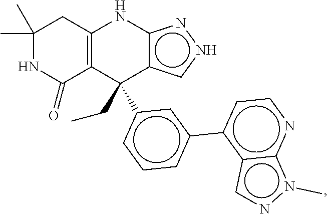 | 948817 |
| 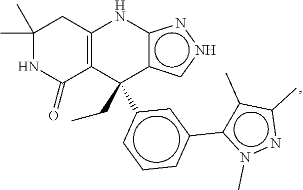 | 948818 |
| 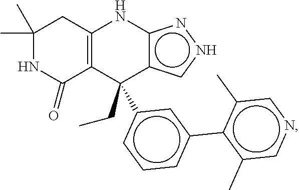 | 948829 |
| 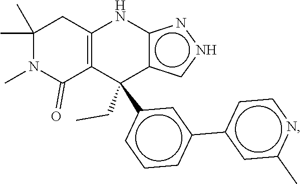 | 949009 |
| 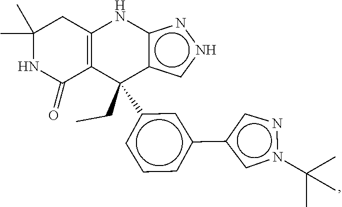 | 949107 |
| 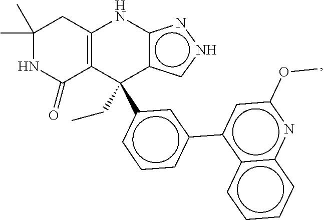 | 949116 |
| 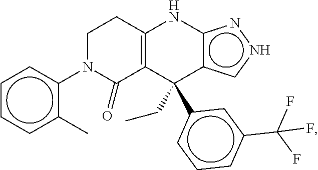 | 949129 |
| 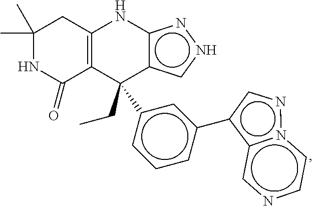 | 949263 |
| 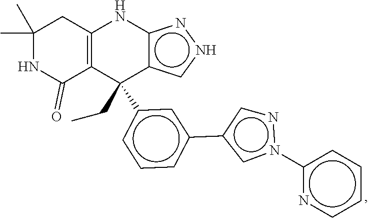 | 949264 |
| 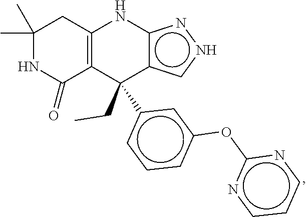 | 949335 |
| 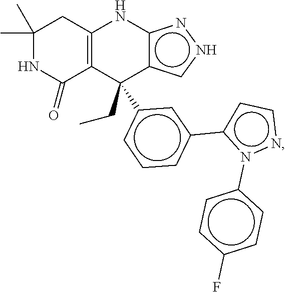 | 949396 |
| 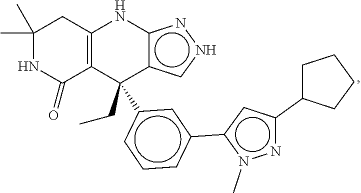 | 949509 |
| 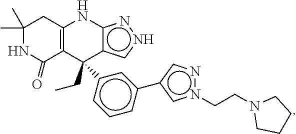 | 949510 |
| 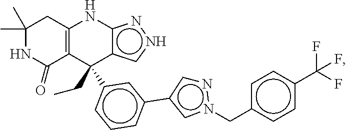 | 949511 |
| 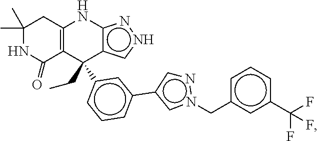 | 949513 |
| 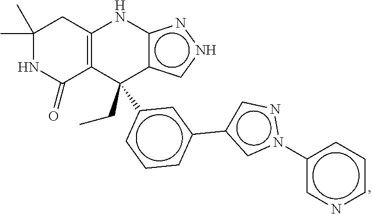 | 949515 |
| 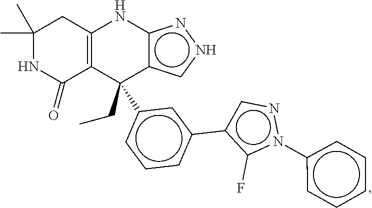 | 949519 |
| 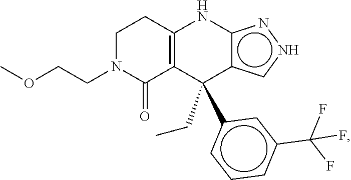 | 949600 |
| 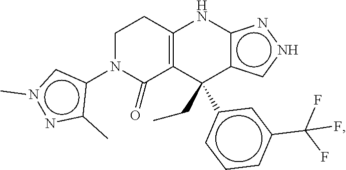 | 949641 |
| 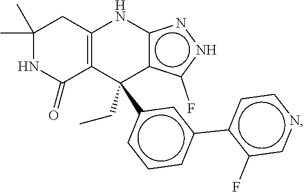 | 949643 |
| 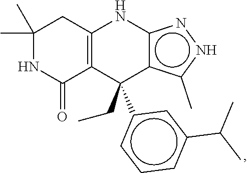 | 949646 |
| 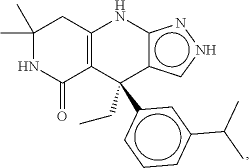 | 949752 |
| 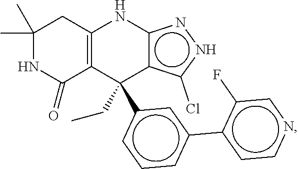 | 949756 |
| 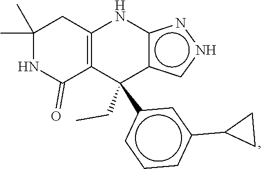 | 950817 |
| 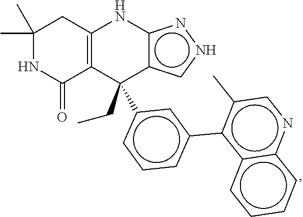 | 950825 |
| 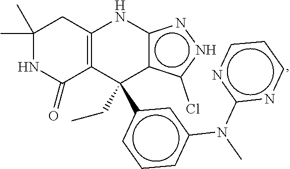 | 951592 |
| 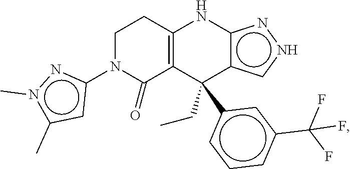 | 951954 |
| 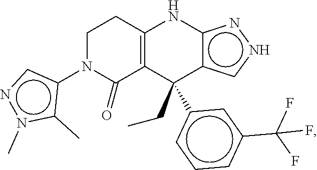 | 952262 |
| 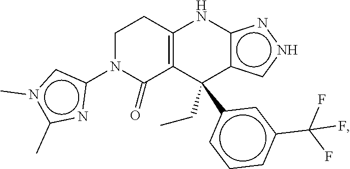 | 952479 |
| 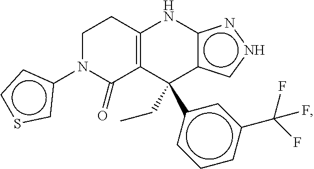 | 952636 |
| 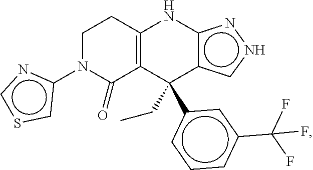 | 952638 |
| 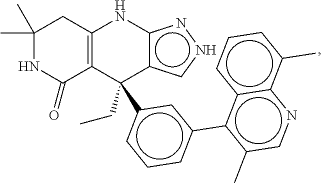 | 952760 |

or

| | |
|---|---|
| 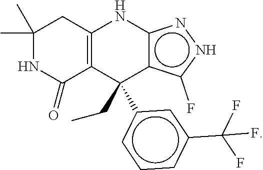 | 957497 |

In some embodiments, Formula I-A or I-B is any one of the formulae shown in Table 2A:

TABLE 2A

| 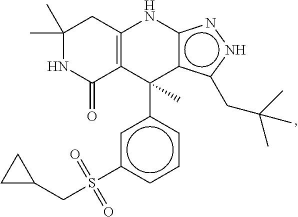 | 108006 |
|---|---|
| 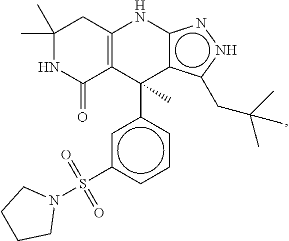 | 108794 |
| or | |
| 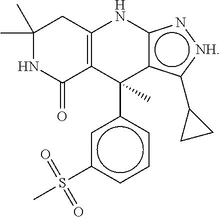 | 114744 |

In some embodiments, Formula I-A or I-B is not any one of the formulae shown in Table 2. In some embodiments, Formula I-A or I-B is not any one of the formulae shown in Table 2A.

In another aspect, provided herein is a compound, or a pharmaceutically acceptable salt, solvate, hydrate, polymorph, co-crystal, tautomer, stereoisomer, isotopically labeled compound, or prodrug thereof, wherein the compound is of Formula II-A or II-B:

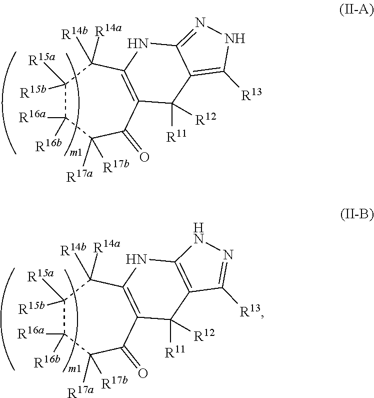

wherein:
each instance of [symbol] is independently a single or double bond;
R¹¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R¹² is optionally substituted alkyl, optionally substituted alkenyl, or optionally substituted alkynyl;
or R¹¹ and R¹² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
R¹³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{B}, —SCN, —SR^{B}, —SSR^{B}, —N₃, —NO, —N(R^{B})₂, —NO₂, —C(═O)R^{B}, —C(═O)OR^{B}, —C(═O)SR^{B}, —C(═O)N(R^{B})₂, —C(═NR^{B})R^{B}, —C(═NR^{B})OR^{B}, —C(═NR^{B})SR^{B}, —C(═NR^{B})N(R^{B})₂, —S(═O)R^{B}, —S(═O)OR^{B}, —S(═O)SR^{B}, —S(═O)N(R^{B})₂, —S(═O)₂R^{B}, —S(═O)₂OR^{B}, —S(═O)₂SR^{B}, —S(═O)₂N(R^{B})₂, —OC(═O)R^{B}, —OC(═O)OR^{B}, —OC(═O)SR^{B}, —OC(═O)N(R^{B})₂, —OC(═NR^{B})R^{B}, —OC(═NR^{B})OR^{B}, —OC(═NR^{B})SR^{B}, —OC(═NR^{B})N(R^{B})₂, —OS(═O)R^{B}, —OS(═O)OR^{B}, —OS(═O)SR^{B}, —OS(═O)N(R^{B})₂, —OS(═O)₂R^{B}, —OS(═O)₂OR^{B}, —OS(═O)₂SR^{B}, —OS(═O)₂N(R^{B})₂, —ON(R^{B})₂, —SC(═O)R^{B}, —SC(═O)OR^{B}, —SC(═O)SR^{B}, —SC(═O)N(R^{B})₂, —SC(═NR^{B})R^{B}, —SC(═NR^{B})OR^{B}, —SC(═NR^{B})SR^{B}, —SC(═NR^{B})N(R^{B})₂, —NR^{B}C(═O)R^{B}, —NR^{B}C(═O)OR^{B}, —NR^{B}C(═O)SR^{B}, —NR^{B}C(═O)N(R^{B})₂, —NR^{B}C(═NR^{B})R^{B}, —NR^{B}C(═NR^{B})OR^{B}, —NR^{B}C(═NR^{B})SR^{B}, —NR^{B}C(═NR^{B})N(R^{B})₂, —NR^{B}S(═O)R^{B}, —NR^{B}S(═O)OR^{B}, —NR^{B}S(═O)SR^{B}, —NR^{B}S(═O)N(R^{B})₂, —NR^{D}S(═O)₂R^{B}, —NR^{B}S(═O)₂OR^{B}, —NR^{B}S(═O)₂SR^{B}, —NR^{B}S(═O)₂N(R^{B})₂, —Si(R^{B})₃, —Si(R^{B})₂OR^{B}, —Si(R^{B})(OR^{B})₂, —Si(OR^{B})₃, —OSi(R^{B})₃, —OSi(R^{B})₂OR^{B}, —OSi(R^{B})(OR^{B})₂, or —OSi(OR^{B})₃;
each of R^{14a}, R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each instance of R^{B} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{B} attached to the same intervening atom are joined together with the intervening atom to form an optionally substituted, monocyclic, heterocyclic or heteroaryl ring; and
or R^{14a} and R^{14b} are taken together with their intervening atom to form an optionally substituted, monocyclic, carbocyclic or heterocyclic ring;
or R^{17a} and R^{17b} are taken together with their intervening atom to form an optionally substituted, monocyclic, carbocyclic or heterocyclic ring;
or R^{14b} and R^{17a} are taken together to form optionally substituted alkylene, optionally substituted alkenylene, optionally substituted alkynylene, optionally substituted heteroalkylene, optionally substituted heteroalkenylene, or optionally substituted heteroalkynylene, each of which independently comprises 1, 2, 3, or 4 backbone atoms; and
m1 is 0 or 1;
when m1 is 1:
   2 each of R^{15a}, R^{15b}, R^{16a} and R^{16b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
   or R^{14b} and R^{15a} are taken together with their intervening atom to form an optionally substituted, monocyclic, carbocyclic, heterocyclic, aryl, or heteroaryl ring;
   or R^{15a} and R^{15b} are taken together with their intervening atom to form an optionally substituted, monocyclic, carbocyclic or heterocyclic ring;
   or R^{15b} and R^{16a} are taken together with their intervening atom to form an optionally substituted, monocyclic, carbocyclic, heterocyclic, aryl, or heteroaryl ring;
   or R^{16a} and R^{16b} are taken together with their intervening atom to form an optionally substituted, monocyclic, carbocyclic or heterocyclic ring;
   or R^{16b} and R^{17a} are taken together with their intervening atom to form an optionally substituted, monocyclic, carbocyclic, heterocyclic, aryl, or heteroaryl ring; provided that the compound is not of the formula:

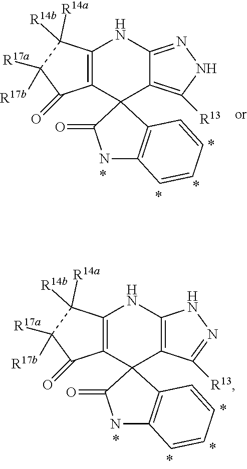

or a pharmaceutically acceptable salt, solvate, hydrate, polymorph, co-crystal, tautomer, stereoisomer, isotopically labeled compound, or prodrug thereof, wherein each instance of the atoms marked with * is independently optionally substituted.

In some embodiments, the compound is of the formula:

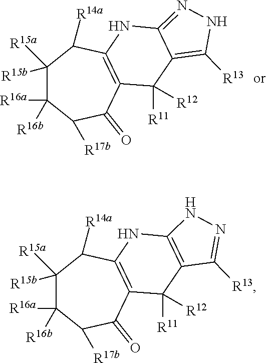

or a pharmaceutically acceptable salt, solvate, hydrate, polymorph, co-crystal, tautomer, stereoisomer, isotopically labeled compound, or prodrug thereof.

In some embodiments, the compound is of the formula:

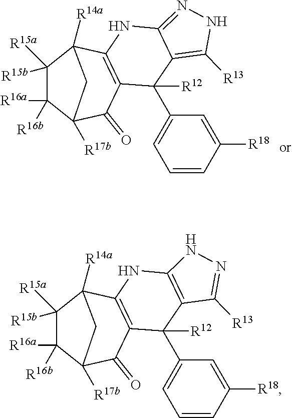

or a pharmaceutically acceptable salt, solvate, hydrate, polymorph, co-crystal, tautomer, stereoisomer, isotopically labeled compound, or prodrug thereof.

In some embodiments, the compound is of the formula:

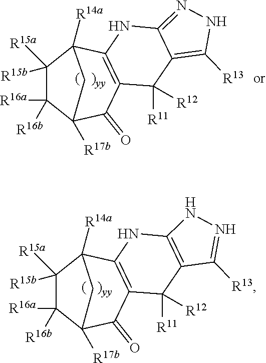

or a pharmaceutically acceptable salt, solvate, hydrate, polymorph, co-crystal, tautomer, stereoisomer, isotopically labeled compound, or prodrug thereof, wherein yy is 2, 3, or 4. In some embodiments, yy is 2 or 3. In some embodiments, yy is 3 or 4. In some embodiments, yy is or 4. In some embodiments, yy is 2. In some embodiments, yy is 3. In some embodiments, yy is 4.

In some embodiments, the compound is of the formula:

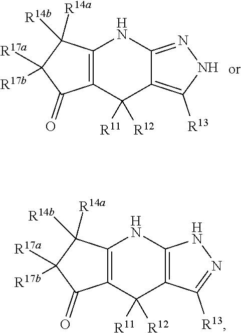

or a pharmaceutically acceptable salt, solvate, hydrate, polymorph, co-crystal, tautomer, stereoisomer, isotopically labeled compound, or prodrug thereof.

In some embodiments, the compound is of the formula:

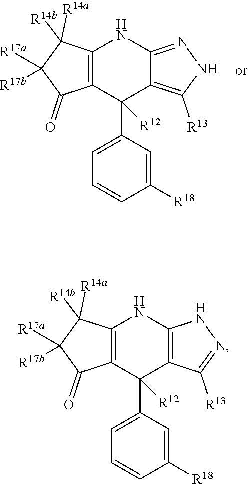

or a pharmaceutically acceptable salt, solvate, hydrate, polymorph, co-crystal, tautomer, stereoisomer, isotopically labeled compound, or prodrug thereof.

In some embodiments, at least one instance of [symbol] is a single bond. In some embodiments, one instance of [symbol] is a single bond. In some embodiments, two instances of [symbol] are single bonds. In some embodiments, each instance of [symbol] is a single bond. In some embodiments, at least one instance of [symbol] is a double bond. In some embodiments, one instance of [symbol] is a double bond. In some embodiments, two instances of [symbol] are double bonds.

In some embodiments, m1 is 0. In some embodiments, m1 is 1.

In some embodiments,

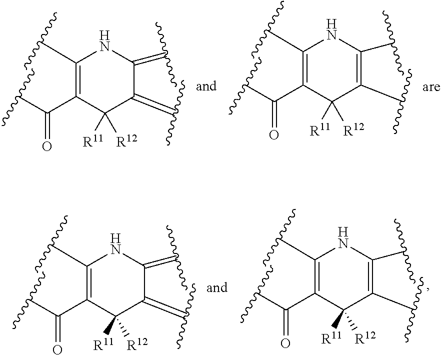

respectively. In some embodiments,

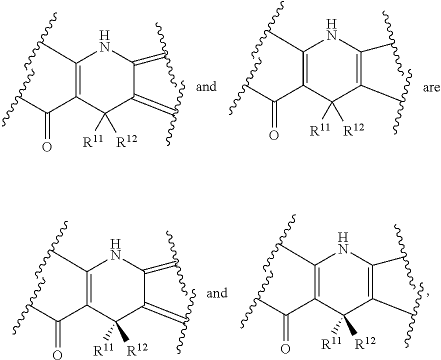

respectively.

In some embodiments, R¹¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl. In some embodiments, R¹¹ is optionally substituted C₁₋₁₀ alkyl, optionally substituted C₁₋₁₀ alkenyl, or optionally substituted C₁₋₁₀ alkynyl.

In some embodiments, R¹¹ is optionally substituted carbocyclyl or optionally substituted heterocyclyl. In some embodiments, R¹¹ is optionally substituted C₃₋₁₄ carbocyclyl. In some embodiments, R¹¹ is optionally substituted monocyclic C₃₋₇ carbocyclyl. In some embodiments, R¹¹ is optionally substituted monocyclic C₃₋₄ carbocyclyl. In some embodiments, R¹¹ is optionally substituted monocyclic C₅₋₇ carbocyclyl. In some embodiments, R¹¹ is saturated carbocyclyl. In some embodiments, R¹¹ is carbocyclyl comprising only one unsaturated bond (e.g., C═C bond) in the carbocyclic ring system.

In some embodiments, R¹¹ is 3- to 14-membered optionally substituted heterocyclyl. In some embodiments, R¹¹ is optionally substituted monocyclic 3- to 7-membered heterocyclyl. In some embodiments, R¹¹ is optionally substituted 3- to 14-membered heterocyclyl comprising one or more O and/or S atoms but no N atoms. In some embodiments, R¹¹ is optionally substituted 3- to 14-membered heterocyclyl comprising one or more N atoms and optionally one or more O and/S atoms. In some embodiments, R¹¹ is optionally substituted monocyclic 3- to 7-membered heterocyclyl comprising one or more O and/or S atoms but no N atoms. In some embodiments, R¹¹ is optionally substituted monocyclic 3- to 7-membered heterocyclyl comprising one or more N atoms and optionally one or more O and/or S atoms. In some embodiments, R¹¹ is saturated heterocyclyl. In some embodiments, R¹¹ is heterocyclyl comprising only one unsaturated bond (e.g., C═C bond) in the heterocyclic ring system.

In some embodiments, R¹¹ is optionally substituted aryl. In some embodiments, R¹¹ is optionally substituted monocyclic aryl. In some embodiments, R¹¹ is optionally substituted bicyclic aryl. In some embodiments, R¹¹ is optionally substituted C₆₋₁₄ aryl. In some embodiments, R¹¹ is optionally substituted C₆₋₁₀ aryl. In some embodiments, R¹ is optionally substituted phenyl. In some embodiments, R¹¹ is optionally substituted naphthyl.

In some embodiments, R¹¹ is optionally substituted heteroaryl. In some embodiments, R¹¹ is optionally substituted monocyclic heteroaryl. In some embodiments, R¹l is optionally substituted bicyclic heteroaryl. In some embodiments, R¹¹ is optionally substituted 5- to 14-membered heteroaryl. In some embodiments, R¹¹ is optionally substituted 5- to 10-membered heteroaryl. In some embodiments, R¹¹ is optionally substituted 5- to 6-membered monocyclic heteroaryl. In some embodiments, R¹¹ is optionally substituted 9- to 10-membered bicyclic heteroaryl.

In some embodiments, R¹¹ is optionally substituted aryl fused with optionally substituted monocyclic C₃₋₇ carbocyclyl. In some embodiments, R¹¹ is optionally substituted C₆₋₁₄ aryl fused with optionally substituted monocyclic C₃₋₇ carbocyclyl. In some embodiments, R¹¹ is optionally substituted C₆₋₁₀ aryl fused with optionally substituted monocyclic C₃₋₇ carbocyclyl. In some embodiments, R¹ is optionally substituted phenyl fused with optionally substituted monocyclic C₃₋₇ carbocyclyl. In some embodiments, R¹¹ is optionally substituted naphthyl fused with optionally substituted monocyclic C₃₋₇ carbocyclyl.

In some embodiments, R¹¹ is optionally substituted aryl fused with optionally substituted monocyclic 3- to 7-membered heterocyclyl. In some embodiments, R¹¹ is optionally substituted C₆₋₁₄ aryl fused with optionally substituted monocyclic 3- to 7-membered heterocyclyl. In some embodiments, R¹¹ is optionally substituted C₆₋₁₀ aryl fused with optionally substituted monocyclic 3- to 7-membered heterocyclyl. In some embodiments, R¹¹ is optionally substituted phenyl fused with optionally substituted monocyclic 3- to 7-membered heterocyclyl. In some embodiments, R¹¹ is optionally substituted naphthyl fused with optionally substituted monocyclic 3- to 7-membered heterocyclyl.

In some embodiments, R¹¹ is optionally substituted heteroaryl fused with optionally substituted monocyclic C₃₋₇ carbocyclyl. In some embodiments, R¹¹ is optionally substituted 5- to 14-membered heteroaryl fused with optionally substituted monocyclic C₃₋₇ carbocyclyl. In some embodiments, R¹¹ is optionally substituted 5- to 10-membered heteroaryl fused with optionally substituted monocyclic C₃₋₇ carbocyclyl. In some embodiments, R¹¹ is optionally substituted 5- to 6-membered monocyclic heteroaryl fused with optionally substituted monocyclic C₃₋₇ carbocyclyl. In some embodiments, R¹¹ is optionally substituted 9- to 10-membered bicyclic heteroaryl fused with optionally substituted monocyclic C₃₋₇ carbocyclyl.

In some embodiments, R¹¹ is optionally substituted heteroaryl fused with optionally substituted monocyclic 3- to 7-membered heterocyclyl. In some embodiments, R¹¹ is optionally substituted 5- to 14-membered heteroaryl fused with optionally substituted monocyclic 3- to 7-membered heterocyclyl. In some embodiments, R¹¹ is optionally substituted 5- to 10-membered heteroaryl fused with optionally substituted monocyclic 3- to 7-membered heterocyclyl. In some embodiments, R¹¹ is optionally substituted 5- to 6-membered monocyclic heteroaryl fused with optionally substituted monocyclic 3- to 7-membered heterocyclyl. In some embodiments, R¹¹ is optionally substituted 9- to 10-membered bicyclic heteroaryl fused with optionally substituted monocyclic 3- to 7-membered heterocyclyl.

In some embodiments, R¹¹ is

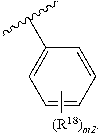

Each instance of R¹⁸ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O) ZN(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃.

m2 is 0, 1, 2, 3, 4, or 5. In some embodiments, m2 is 0, 1, 2, 3, or 4. In some embodiments, m2 is 0, 1, 2, or 3. In some embodiments, m2 is 0, 1, or 2. In some embodiments, m2 is 0 or 1. In some embodiments, m2 is 0. In some embodiments, m2 is 1. In some embodiments, m2 is 2. In some embodiments, m2 is 3. In some embodiments, m2 is 4. In some embodiments, m2 is 5.

In some embodiments, R¹¹ is

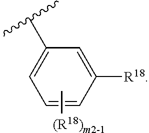

In some embodiments, R¹¹ is

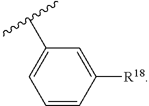

In some embodiments, R¹¹ is

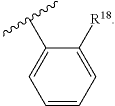

In some embodiments, R¹¹ is

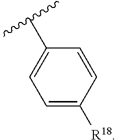

In some embodiments, R¹¹ is

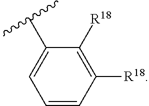

In some embodiments, R¹ is

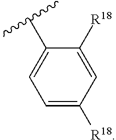

In some embodiments, R¹¹ is

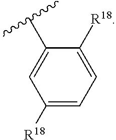

In some embodiments, R¹¹ is

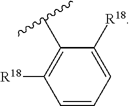

In some embodiments, R¹¹ is

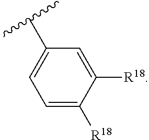

In some embodiments, R¹¹ is

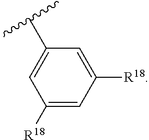

In some embodiments, at least one instance of R¹⁸ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{D}, —SR^{D}, or —N(R^{D})₂. In some embodiments, at least one instance of R¹⁸ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, at least one instance of R¹⁸ is C₁₋₁₀ haloalkyl. In some embodiments, at least one instance of R¹⁸ is C₁₋₄ haloalkyl. In some embodiments, at least one instance of R¹⁸ is C₁₋₄ fluoroalkyl (e.g., C₁₋₄ perfluoroalkyl). In some embodiments, at least one instance of R¹⁸ is —CF₃.

In some embodiments, at least one instance of R¹⁸ is halogen. In some embodiments, at least one instance of R¹⁸ is bromine, chlorine, or fluorine. In some embodiments, at least one instance of R¹⁸ is bromine or chlorine. In some embodiments, at least one instance of R¹⁸ is chlorine or fluorine. In some embodiments, at least one instance of R¹² is bromine. In some embodiments, at least one instance of R¹³ is chlorine. In some embodiments, at least one instance of R¹⁸ is fluorine.

In some embodiments, at least one instance of R¹⁸ is —OR^{D}, —SR^{D}, or —N(R^{D})₂ (e.g., wherein R^{D} is hydrogen or optionally substituted alkyl, or optionally substituted phenyl). In some embodiments, at least one instance of R¹⁸ is —OH. In some embodiments, at least one instance of R¹⁸ is —SH. In some embodiments, at least one instance of R¹⁸ is —NH₂. In some embodiments, at least one instance of R¹⁸ is —NHR^{D}. In some embodiments, at least one instance of R¹⁸ is —NMeR^{D}. In some embodiments, at least one instance of R¹⁸ is —N(heteroaryl) R^{D}. In some embodiments, at least one instance of R¹⁸ is —CN, —SCN, —SSR^{D}, —N₃, —NO, or —NO₂. In some embodiments, at least one instance of R¹⁸ is —C(═O)R^{D}, —C(═O)OR^{D}, —C(═O)SR^{D}, or —C(═O)N(R^{D})₂ (e.g., wherein R^{D} is hydrogen or optionally substituted alkyl, or optionally substituted phenyl). In some embodiments, at least one instance of R¹⁸ is —C(═NR^{D})R^{D}, —C(═NR^{D})OR^{D}, —C(═NR^{D})SR^{D}, or —C(═NR^{D})N(R^{D})₂ (e.g., wherein R^{D} is hydrogen or optionally substituted alkyl, or optionally substituted phenyl). In some embodiments, at least one instance of R¹⁸ is —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, or —S(═O)₂N(R^{D})₂ (e.g., wherein R^{D} is hydrogen or optionally substituted alkyl, or optionally substituted phenyl). In some embodiments, at least one instance of R¹⁸ is —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, or —ON(R^{D})₂ (e.g., wherein R^{D} is hydrogen or optionally substituted alkyl, or optionally substituted phenyl). In some embodiments, at least one instance of R¹⁸ is —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, or —SC(═NR^{D})N(R^{D})₂ (e.g., wherein R^{D} is hydrogen or optionally substituted alkyl, or optionally substituted phenyl). In some embodiments, at least one instance of R¹⁸ is —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(NR^{D})OR^{D}, —NR^{D}C(NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, or —NR^{D}S(═O)₂N(R^{D})₂ (e.g., wherein R^{D} is hydrogen or optionally substituted alkyl, or optionally substituted phenyl). In some embodiments, at least one instance of R¹⁸ is —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃ (e.g., wherein R^{D} is hydrogen or optionally substituted alkyl, or optionally substituted phenyl).

In some embodiments, at least one instance of R¹⁸ is optionally substituted carbocyclyl, optionally substituted heterocyclyl, optionally substituted aryl, or optionally substituted heteroaryl. In some embodiments, at least one instance of R¹⁸ is optionally substituted C₃₋₁₄ carbocyclyl. In some embodiments, at least one instance of R¹⁸ is optionally substituted monocyclic C₃₋₇ carbocyclyl. In some embodiments, at least one instance of R¹⁸ is optionally substituted monocyclic C₃₋₄ carbocyclyl. In some embodiments, at least one instance of R¹⁸ is optionally substituted monocyclic C₅₋₇ carbocyclyl. In some embodiments, at least one instance of R¹⁸ is saturated carbocyclyl. In some embodiments, at least one instance of R¹⁸ is carbocyclyl comprising only one unsaturated bond (e.g., C═C bond) in the carbocyclic ring system.

In some embodiments, at least one instance of R¹⁸ is optionally substituted 3- to 14-membered heterocyclyl. In some embodiments, at least one instance of R¹⁸ is optionally substituted monocyclic 3- to 7-membered heterocyclyl. In some embodiments, at least one instance of R¹⁸ is optionally substituted 3- to 14-membered heterocyclyl comprising one or more O and/or S atoms but no N atoms. In some embodiments, at least one instance of R¹⁸ is optionally substituted 3- to 14-membered heterocyclyl comprising one or more N atoms and optionally one or more O and/S atoms. In some embodiments, at least one instance of R¹⁸ is optionally substituted monocyclic 3- to 7-membered heterocyclyl comprising one or more O and/or S atoms but no N atoms. In some embodiments, at least one instance of R¹⁸ is optionally substituted monocyclic 3- to 7-membered heterocyclyl comprising one or more N atoms and optionally one or more O and/or S atoms. In some embodiments, at least one instance of R¹⁸ is saturated heterocyclyl. In some embodiments, at least one instance of R¹⁸ is heterocyclyl comprising only one unsaturated bond (e.g., C═C bond) in the heterocyclic ring system.

In some embodiments, at least one instance of R¹⁸ is optionally substituted monocyclic aryl. In some embodiments, at least one instance of R¹⁸ is optionally substituted bicyclic aryl. In some embodiments, at least one instance of R¹⁸ is optionally substituted C₆₋₁₄ aryl. In some embodiments, at least one instance of R¹⁸ is optionally substituted C₆₋₁₀ aryl. In some embodiments, at least one instance of R¹⁸ is optionally substituted phenyl. In some embodiments, at least one instance of R¹⁸ is optionally substituted naphthyl.

In some embodiments, at least one instance of R¹⁸ is optionally substituted monocyclic heteroaryl. In some embodiments, at least one instance of R¹⁸ is optionally substituted bicyclic heteroaryl. In some embodiments, at least one instance of R¹⁸ is optionally substituted 5- to 14-membered heteroaryl. In some embodiments, at least one instance of R¹⁸ is optionally substituted 5- to 10-membered heteroaryl. In some embodiments, at least one instance of R¹⁸ is optionally substituted 5- to 6-membered monocyclic heteroaryl. In some embodiments, at least one instance of R¹⁸ is optionally substituted heteroaryl comprising one or more N atoms. In some embodiments, at least one instance of R¹⁸ is optionally substituted pyridinyl, optionally substituted triazolyl, or optionally substituted pyrazolyl. In some embodiments, at least one instance of R¹⁸ is optionally substituted pyridinyl. In some embodiments, at least one instance of R¹⁸ is optionally substituted pyrazolyl.

In some embodiments, at least one instance of R^{D} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{D} is optionally substituted carbocyclyl, optionally substituted heterocyclyl, optionally substituted aryl, or optionally substituted heteroaryl. In some embodiments, at least one instance of R^{D} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{D} is independently hydrogen, optionally substituted C₁₋₁₀ alkyl, or optionally substituted phenyl. In some embodiments, at least one instance of R^{D} is a nitrogen protecting group when attached to a nitrogen atom, an oxygen protecting group when attached to an oxygen atom, or a sulfur protecting group when attached to a sulfur atom. In some embodiments, two instances of R^{D} attached to the same intervening atom are joined together with the intervening atom to form an optionally substituted, monocyclic, heterocyclic or heteroaryl ring.

In some embodiments, R¹² is optionally substituted alkyl, optionally substituted alkenyl, or optionally substituted alkynyl. In some embodiments, R¹² is optionally substituted C₁₋₄ alkyl, optionally substituted C₁₋₄ alkenyl, or optionally substituted C₁₋₄ alkynyl. In some embodiments, R¹² is optionally substituted alkyl. In some embodiments, R¹² is optionally substituted C₁₋₄ alkyl. In some embodiments, R¹² is unsubstituted C₁-C₄ alkyl. In some embodiments, R¹² is unsubstituted methyl. In some embodiments, R¹² is unsubstituted ethyl.

In some embodiments, R¹¹ and R¹² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring. In some embodiments, R¹¹ and R¹² are taken together with their intervening atom to form an optionally substituted, monocyclic, carbocyclic ring. In some embodiments, R¹¹ and R¹² are taken together with their intervening atom to form an optionally substituted, monocyclic, 3- to 7-membered carbocyclic ring. In some embodiments, R¹¹ and R¹² are taken together with their intervening atom to form an optionally substituted, monocyclic, 5- to 6-membered carbocyclic ring. In some embodiments, R¹ and R¹² are taken together with their intervening atom to form an optionally substituted, monocyclic, heterocyclic ring. In some embodiments, R¹¹ and R¹² are taken together with their intervening atom to form an optionally substituted, monocyclic, 3- to 7-membered heterocyclic ring. In some embodiments, R¹¹ and R¹² are taken together with their intervening atom to form an optionally substituted, monocyclic, 5- to 6-membered heterocyclic ring. In some embodiments, R¹¹ and R¹² are taken together with their intervening atom to form an optionally substituted, monocyclic, heterocyclic ring comprising one or more N atoms. In some embodiments, R¹¹ and R¹² are taken together with their intervening atom to form an optionally substituted, monocyclic, 3- to 7-membered heterocyclic ring comprising one or more N atoms. In some embodiments, R¹¹ and R¹² are taken together with their intervening atom to form optionally substituted tetrahydropyranyl.

In some embodiments, R¹³ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{B}, —SR^{B}, or —N(R^{B})₂. In some embodiments, R¹³ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₀ heteroalkynyl. In some embodiments, R¹³ is C₁₋₁₀ haloalkyl. In some embodiments, R¹³ is C₁₋₄ haloalkyl. In some embodiments, R¹³ is C₁₋₄ fluoroalkyl (e.g., C₁₋₄ perfluoroalkyl). In some embodiments, R¹³ is —CF₃. In some embodiments, R¹³ is hydrogen, optionally substituted C₁-C₆ alkyl, or halogen. In some embodiments, R¹³ is hydrogen, fluorine, —CH₃, —CH₂F, —CHF₂, or —CF₃. In some embodiments, R¹³ is hydrogen, fluorine, —CH₃, or —CF₃. In some embodiments, R¹³ is hydrogen or halogen. In some embodiments, R¹³ is hydrogen or fluorine. In some embodiments, R¹³ is hydrogen or optionally substituted C₁-C₆ alkyl. In some embodiments, R¹³ is hydrogen, unsubstituted C₁-C₆ alkyl, or C₁₋₆ haloalkyl. In some embodiments, R¹³ is hydrogen.

In some embodiments, R¹³ is halogen. In some embodiments, R¹³ is bromine, chlorine, or fluorine. In some embodiments, R¹³ is bromine or chlorine. In some embodiments, R¹³ is chlorine or fluorine. In some embodiments, R¹³ is bromine. In some embodiments, R¹³ is chlorine. In some embodiments, R¹³ is fluorine.

In some embodiments, R¹³ is —OR^{B}, —SR^{B}, or —N(R^{B})₂ (e.g., wherein R^{B} is hydrogen or optionally substituted alkyl, or optionally substituted phenyl). In some embodiments, R¹³ is —OH. In some embodiments, R¹³ is —SH. In some embodiments, R¹³ is —NH₂. In some embodiments, R¹³ is —CN, —SCN, —SSR^{B}, —N₃, —NO, or —NO₂. In some embodiments, R¹³ is —C(═O)R^{B}, —C(═O)OR^{B}, —C(═O)SR^{B}, or —C(═O)N(R^{B})₂ (e.g., wherein R^{B} is hydrogen or optionally substituted alkyl, or optionally substituted phenyl). In some embodiments, R¹³ is —C(═NR^{B})R^{B}, —C(═NR^{B})OR^{B}, —C(═NR^{B})SR^{B}, or —C(═NR^{B})N(R^{B})₂ (e.g., wherein R^{B} is hydrogen or optionally substituted alkyl, or optionally substituted phenyl). In some embodiments, R¹³ is —S(═O)R^{B}, —S(═O)OR^{B}, —S(═O)SR^{B}, —S(═O)N(R^{B})₂, —S(═O)₂R^{B}, —S(═O)₂OR^{B}, —S(═O)₂SR^{B}, or —S(═O)₂N(R^{B})₂ (e.g., wherein R^{B} is hydrogen or optionally substituted alkyl, or optionally substituted phenyl). In some embodiments, R¹³ is —OC(═O)R^{B}, —OC(═O)OR^{B}, —OC(═O)SR^{B}, —OC(═O)N(R^{B})₂, —OC(═NR^{B})R^{B}, —OC(═NR^{B})OR^{B}, —OC(═NR^{B})SR^{B}, —OC(═NR^{B})N(R^{B})₂, —OS(═O)R^{B}, —OS(═O)OR^{B}, —OS(═O)SR^{B}, —OS(═O)N(R^{B})₂, —OS(═O)₂R^{B}, —OS(═O)₂OR^{B}, —OS(═O)₂SR^{B}, —OS(═O)₂N(R^{B})₂, or —ON(R^{B})₂ (e.g., wherein R^{B} is hydrogen or optionally substituted alkyl, or optionally substituted phenyl). In some embodiments, R¹³ is —SC(═O)R^{B}, —SC(═O)OR^{B}, —SC(═O)SR^{B}, —SC(═O)N(R^{B})₂, —SC(═NR^{B})R^{B}, —SC(═NR^{B})OR^{B}, —SC(═NR^{B})SR^{B}, or —SC(═NR^{B})N(R^{B})₂ (e.g., wherein R^{B} is hydrogen or optionally substituted alkyl, or optionally substituted phenyl). In some embodiments, R¹³ is —NR^{B}C(═O)R^{B}, —NR^{B}C(═O)OR^{B}, —NR^{B}C(═O)SR^{B}, —NR^{B}C(═O)N(R^{B})₂, —NR^{B}C(═NR^{B})R^{B}, —NR^{B}C(═NR^{B})OR^{B}, —NR^{B}C(═NR^{B})SR^{B}, —NR^{B}C(═NR^{B})N(R^{B})₂, —NR^{B}S(═O)R^{B}, —NR^{B}S(═O)OR^{B}, —NR^{B}S(═O)SR^{B}, —NR^{B}S(═O)N(R^{B})₂, —NR^{B}S(═O)₂R^{B}, —NR^{B}S(═O)₂OR^{B}, —NR^{B}S(═O)₂SR^{B}, or —NR^{B}S(═O)₂N(R^{B})₂ (e.g., wherein R^{B} is hydrogen or optionally substituted alkyl, or optionally substituted phenyl). In some embodiments, R¹³ is —Si(R^{B})₃, —Si(R^{B})₂OR^{B}, —Si(R^{B})(OR^{B})₂, —Si(OR^{B})₃, —OSi(R^{B})₃, —OSi(R^{B})₂OR^{B}, —OSi(R^{B})(OR^{B})₂, or —OSi(OR^{B})₃ (e.g., wherein R^{B} is hydrogen or optionally substituted alkyl, or optionally substituted phenyl).

In some embodiments, R¹³ is optionally substituted carbocyclyl, optionally substituted heterocyclyl, optionally substituted aryl, or optionally substituted heteroaryl. In some embodiments, R¹³ is optionally substituted C₃₋₁₄ carbocyclyl. In some embodiments, R¹³ is optionally substituted monocyclic C₃₋₇ carbocyclyl. In some embodiments, R¹³ is optionally substituted monocyclic C₃₋₄ carbocyclyl. In some embodiments, R¹³ is optionally substituted monocyclic C₅₋₇ carbocyclyl. In some embodiments, R¹³ is saturated carbocyclyl. In some embodiments, R¹³ is carbocyclyl comprising only one unsaturated bond (e.g., C═C bond) in the carbocyclic ring system.

In some embodiments, R¹³ is optionally substituted 3- to 14-membered heterocyclyl. In some embodiments, R¹³ is optionally substituted monocyclic 3- to 7-membered heterocyclyl. In some embodiments, R¹³ is optionally substituted 3- to 14-membered heterocyclyl comprising one or more O and/or S atoms but no N atoms. In some embodiments, R¹³ is optionally substituted 3-14 to 14-membered heterocyclyl comprising one or more N atoms and optionally one or more O and/S atoms. In some embodiments, R³³ is optionally substituted monocyclic 3- to 7-membered heterocyclyl comprising one or more O and/or S atoms but no N atoms. In some embodiments, R¹³ is optionally substituted monocyclic 3- to 7-membered heterocyclyl comprising one or more N atoms and optionally one or more O and/or S atoms. In some embodiments, R¹³ is saturated heterocyclyl. In some embodiments, R¹³ is heterocyclyl comprising only one unsaturated bond (e.g., C═C bond) in the heterocyclic ring system.

In some embodiments, R¹³ is optionally substituted monocyclic aryl. In some embodiments, R¹³ is optionally substituted bicyclic aryl. In some embodiments, R¹³ is optionally substituted C₆₋₁₄ aryl. In some embodiments, R¹³ is optionally substituted C₆₋₁₀ aryl. In some embodiments, R¹³ is optionally substituted phenyl. In some embodiments, R¹³ is optionally substituted naphthyl.

In some embodiments, R¹³ is optionally substituted monocyclic heteroaryl. In some embodiments, R¹³ is optionally substituted bicyclic heteroaryl. In some embodiments, R¹³ is optionally substituted 5- to 14-membered heteroaryl. In some embodiments, R¹³ is optionally substituted 5- to 10-membered heteroaryl. In some embodiments, R¹³ is optionally substituted 5- to 6-membered monocyclic heteroaryl.

In some embodiments, at least one instance of R^{B} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{B} is optionally substituted carbocyclyl, optionally substituted heterocyclyl, optionally substituted aryl, or optionally substituted heteroaryl. In some embodiments, at least one instance of R^{B} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{B} is independently hydrogen, optionally substituted C₁₋₁₀ alkyl, or optionally substituted phenyl. In some embodiments, at least one instance of R^{B} is a nitrogen protecting group when attached to a nitrogen atom, an oxygen protecting group when attached to an oxygen atom, or a sulfur protecting group when attached to a sulfur atom. In some embodiments, two instances of R^{B} attached to the same intervening atom are joined together with the intervening atom to form an optionally substituted, monocyclic, heterocyclic or heteroaryl ring.

In some embodiments, each of R^{14a}. R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, each of R^{14a}, R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, each of R^{14a}, R^{14b}, R^{17a}, and R^{17b} is independently hydrogen, halogen, or optionally substituted alkyl. In some embodiments, each of R^{14a}, R^{14b}, R^{17a}, and R^{17b} is independently hydrogen, halogen, or optionally substituted C₁₋₄ alkyl. In some embodiments, each of R^{14a}, R^{14b}, R^{17a}, and R^{17b} is independently hydrogen or optionally substituted C₁₋₄ alkyl. In some embodiments, at least one of R^{14a}, R^{14b}, R^{17a}, and R^{17b} is hydrogen. In some embodiments, each of R^{14a}, R^{14b}, R^{17a} and R^{17b} is hydrogen. In some embodiments, at least one of R^{14a}, R^{14b}, R^{17a}, and R^{17b} is optionally substituted C₁₋₄ alkyl. In some embodiments, at least one of R^{14a}, R^{14b}, R^{17a}, and R^{17b} is unsubstituted methyl.

In some embodiments, R^{14a} and R^{14b} are independently hydrogen or optionally substituted C₁₋₆ alkyl. In some embodiments, at least one of R^{14a} and R^{14b} is hydrogen. In some embodiments, R^{14a} and R^{14b} are hydrogen. In some embodiments, at least one of R^{14a} and R^{14b} is independently optionally substituted C₁₋₆ alkyl. In some embodiments, R^{14a} and R^{14b} are independently optionally substituted C₁₋₆ alkyl.

In some embodiments, R^{17a} and R^{17b} are independently hydrogen or optionally substituted C₁₋₆ alkyl. In some embodiments, at least one of R^{17a} and R^{17b} is hydrogen. In some embodiments, R^{17a} and R^{17b} are hydrogen. In some embodiments, at least one of R^{17a} and R^{17b} is independently optionally substituted C₁₋₆ alkyl. In some embodiments, R^{17a} and R^{17b} are independently optionally substituted C₁₋₆ alkyl. In some embodiments, at least one of R^{17a} and R^{17b} is unsubstituted methyl. In some embodiments, R^{17a} and R^{17b} are unsubstituted methyl.

In some embodiments, R^{14a} and R^{14b} are taken together with their intervening atom to form an optionally substituted, monocyclic, carbocyclic or heterocyclic ring. In some embodiments, R^{14a} and R¹⁴ are taken together with their intervening atom to form an optionally substituted, monocyclic, C₃₋₁₄ carbocyclic ring. In some embodiments, R^{14a} and R^{14b} are taken together with their intervening atom to form an optionally substituted, monocyclic, C₃₋₇ carbocyclic ring. In some embodiments, R^{14a} and R^{14b} are taken together with their intervening atom to form an optionally substituted, monocyclic, 3- to 14-membered heterocyclic ring. In some embodiments, R^{14a} and R^{14b} are taken together with their intervening atom to form an optionally substituted, monocyclic, 3- to 7-membered heterocyclic ring.

In some embodiments, R^{17a} and R^{17b} are taken together with their intervening atom to form an optionally substituted, monocyclic, carbocyclic or heterocyclic ring. In some embodiments, R^{17a} and R^{17b} are taken together with their intervening atom to form an optionally substituted, monocyclic, C₃₋₁₄ carbocyclic ring. In some embodiments, R^{17a} and R^{17b} are taken together with their intervening atom to form an optionally substituted, monocyclic, C₃₋₇ carbocyclic ring. In some embodiments, R^{17a} and R^{17b} are taken together with their intervening atom to form an optionally substituted, monocyclic, 3- to 14-membered heterocyclic ring. In some embodiments, R^{17a} and R^{17b} are taken together with their intervening atom to form an optionally substituted, monocyclic, 3- to 7-membered heterocyclic ring.

In some embodiments, R^{14b} and R^{17a} are taken together to form optionally substituted alkylene, optionally substituted alkenylene, or optionally substituted alkynylene, each of which independently comprises 1, 2, 3, or 4 backbone atoms. In some embodiments, R^{14b} and R^{17a} are taken together to form optionally substituted alkylene or optionally substituted alkenylene, each of which independently comprises 1, 2, 3, or 4 backbone atoms. In some embodiments, R^{14b} and R^{17a} are taken together to form optionally substituted alkylene, comprising 1 or 2 backbone atoms. In some embodiments, R^{14b} and R^{17a} are taken together to form optionally substituted alkylene, comprising 1 backbone atom. In some embodiments, R^{14b} and R^{17a} are taken together to form optionally substituted alkylene, comprising 2 backbone atoms. In some embodiments, R^{14b} and R^{17a} are taken together to form optionally substituted alkenylene, comprising 3 or 4 backbone atoms.

In some embodiments, R^{14b} and R^{17a} are taken together to form optionally substituted heteroalkylene, optionally substituted heteroalkenylene, or optionally substituted heteroalkynylene, each of which independently comprises 1, 2, 3, or 4 backbone atoms. In some embodiments, R^{14b} and R^{17a} are taken together to form optionally substituted heteroalkylene or optionally substituted heteroalkenylene, each of which independently comprises 1, 2, 3, or 4 backbone atoms. In some embodiments, R^{14b} and R^{17a} are taken together to form optionally substituted heteroalkylene, comprising 1 or 2 backbone atoms. In some embodiments, R^{14b} and R^{17a} are taken together to form optionally substituted heteroalkylene, comprising 1 backbone atom. In some embodiments, R^{14b} and R^{17a} are taken together to form optionally substituted heteroalkylene, comprising 2 backbone atoms. In some embodiments, R^{14b} and R^{17a} are taken together to form optionally substituted heteroalkenylene, comprising 3 or 4 backbone atoms.

In some embodiments, each of R^{15a}, R^{15a}, R^{16a} and R^{16b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, each of R^{15a}, R^{15b}, R^{16a} and R^{16b} is independently hydrogen, halogen, or optionally substituted alkyl. In some embodiments, each of R^{15a}, R^{15b}, R^{16a} and R^{16b} is independently hydrogen, —CH₃, or —CF₃. In some embodiments, at least one of R^{15a}, R^{15b}, R^{16a} and R^{16b} is hydrogen. In some embodiments, at least one of R^{15a}, R^{15b}, R^{16a} and R^{16b} is independently optionally substituted C₁₋₁₀ alkyl, optionally substituted C₁₋₁₀ alkenyl, or optionally substituted C₁₋₁₀ alkynyl. In some embodiments, at least one of R^{15a}, R^{15b}, R^{16a} and R^{16b} is independently optionally substituted C₁₋₁₀ heteroalkyl, optionally substituted C₁₋₁₀ heteroalkenyl, or optionally substituted C₁₋₁₀ heteroalkynyl. In some embodiments, at least one of R^{15a}, R^{15b}, R^{16a} and R^{16b} is independently optionally substituted C₁₋₁₀ alkyl or optionally substituted C₁₋₁₀ heteroalkyl. In some embodiments, at least one of R^{15a}. R^{15b}, R^{16a} and R^{16b} is optionally substituted C₁₋₄ alkyl. In some embodiments, at least one of R^{15a}, R^{15b}, R^{16a} and R^{16b} is —CH₃. In some embodiments, at least one of R^{15a}, R^{15b}, R^{16a} and R^{16b} is —CF₃. In some embodiments, at least one of R^{15a}. R^{15b}, R^{16a} and R^{16b} is optionally substituted C₁₋₄ heteroalkyl. In some embodiments, at least one of R^{15a}, R^{15b}, R^{16a} and R^{16b} is optionally substituted C₁₋₄ heteroalkyl comprising an O atom. In some embodiments, at least one of R^{15a}. R^{15b}, R^{16a} and R^{16b} is optionally substituted C₆₋₁₄ aryl. In some embodiments, at least one of R^{15a}, R^{15b}, R^{16a} and R^{16b} is optionally substituted C₆₋₁₀ aryl. In some embodiments, at least one of R^{15a}, R^{15b}, Rica and R^{16b} is optionally substituted phenyl. In some embodiments, each of R^{15a}, R^{15b}, R^{16a} and R^{16b} is hydrogen.

In some embodiments, R^{14b} and R^{15a} are taken together with their intervening atom to form an optionally substituted, monocyclic, carbocyclic, heterocyclic, aryl, or heteroaryl ring. In some embodiments, R^{14b} and R^{15a} are taken together with their intervening atom to form an optionally substituted, monocyclic, carbocyclic or heterocyclic ring. In some embodiments, R^{14b} and R^{15a} are taken together with their intervening atom to form an optionally substituted, monocyclic, C₃₋₁₄ carbocyclic ring. In some embodiments, R^{14b} and R^{15a} are taken together with their intervening atom to form an optionally substituted, monocyclic, C₃₋₇ carbocyclic ring. In some embodiments, R^{14b} and R^{15a} are taken together with their intervening atom to form an optionally substituted, monocyclic, 3- to 14-membered heterocyclic ring. In some embodiments, R^{14b} and R^{15a} are taken together with their intervening atom to form an optionally substituted, monocyclic, 3- to 7-membered heterocyclic ring. In some embodiments, R^{14b} and R^{15a} are taken together with their intervening atom to form an optionally substituted, monocyclic, aryl, or heteroaryl ring. In some embodiments, R^{14b} and R^{15a} are taken together with their intervening atom to form an optionally substituted phenyl ring. In some embodiments, R^{14b} and R^{15a} are taken together with their intervening atom to form an optionally substituted, monocyclic, 5- or 6-membered heteroaryl ring.

In some embodiments, R^{15a} and R^{15b} are taken together with their intervening atom to form an optionally substituted, monocyclic, carbocyclic or heterocyclic ring. In some embodiments, R^{15a} and R^{15b} are taken together with their intervening atom to form an optionally substituted, monocyclic, carbocyclic ring. In some embodiments, R^{15a} and R^{15b} are taken together with their intervening atom to form an optionally substituted, monocyclic, C₃₋₁₄ carbocyclic ring. In some embodiments, R^{15a} and R^{15b} are taken together with their intervening atom to form an optionally substituted, monocyclic, C₃₋₇ carbocyclic ring. In some embodiments, R^{15a} and R^{15b} are taken together with their intervening atom to form an optionally substituted cyclopropyl, cyclobutyl, cyclopentyl, or cyclohexyl ring. In some embodiments, R^{15a} and R^{15b} are taken together with their intervening atom to form an optionally substituted, monocyclic, heterocyclic ring. In some embodiments, R^{15a} and R^{15b} are taken together with their intervening atom to form an optionally substituted, monocyclic, heterocyclic ring comprising O and/or S heteroatom(s) as the only heteroatoms in the heterocyclic ring. In some embodiments, R^{15a} and R^{15b} are taken together with their intervening atom to form an optionally substituted, monocyclic, heterocyclic ring comprising O and/or N heteroatom(s) as the only heteroatoms in the heterocyclic ring. In some embodiments, R^{15a} and R^{15b} are taken together with their intervening atom to form an optionally substituted, monocyclic, 3- to 14-membered heterocyclic ring. In some embodiments, R^{15a} and R^{15b} are taken together with their intervening atom to form an optionally substituted, monocyclic, 3- to 7-membered heterocyclic ring. In some embodiments, R^{15a} and R^{15b} are taken together with their intervening atom to form an optionally substituted, monocyclic, 5- to 6-membered heterocyclic ring.

In some embodiments, R^{15b} and R^{16a} are taken together with their intervening atom to form an optionally substituted, monocyclic, carbocyclic, heterocyclic, aryl, or heteroaryl ring. In some embodiments, R^{15b} and R^{16a} are taken together with their intervening atom to form an optionally substituted, monocyclic, carbocyclic or heterocyclic ring. In some embodiments, R^{15b} and R^{16a} are taken together with their intervening atom to form an optionally substituted, monocyclic, C₃₋₁₄ carbocyclic ring. In some embodiments, R^{15b} and R^{16a} are taken together with their intervening atom to form an optionally substituted, monocyclic, C₃₋₇ carbocyclic ring. In some embodiments, R^{15b} and R^{16a} are taken together with their intervening atom to form an optionally substituted, monocyclic, 3- to 14-membered heterocyclic ring. In some embodiments, R^{15b} and R^{16a} are taken together with their intervening atom to form an optionally substituted, monocyclic, 3- to 7-membered heterocyclic ring. In some embodiments, R^{15b} and R^{16a} are taken together with their intervening atom to form an optionally substituted, monocyclic, aryl, or heteroaryl ring. In some embodiments, R^{15b} and R^{16a} are taken together with their intervening atom to form an optionally substituted phenyl ring. In some embodiments, R^{15b} and R^{16a} are taken together with their intervening atom to form an optionally substituted, monocyclic, 5- or 6-membered heteroaryl ring.

In some embodiments, R^{16a} and R^{16b} are taken together with their intervening atom to form an optionally substituted, monocyclic, carbocyclic or heterocyclic ring. In some embodiments, R^{16a} and R^{16b} are taken together with their intervening atom to form an optionally substituted, monocyclic, carbocyclic ring. In some embodiments, R^{16a} and R^{16b} are taken together with their intervening atom to form an optionally substituted, monocyclic, C₃₋₁₄ carbocyclic ring. In some embodiments, R^{16a} and R^{16b} are taken together with their intervening atom to form an optionally substituted, monocyclic, C₃₋₇ carbocyclic ring. In some embodiments, R^{16a} and R^{16b} are taken together with their intervening atom to form an optionally substituted cyclopropyl, cyclobutyl, cyclopentyl, or cyclohexyl ring. In some embodiments, R^{16a} and R^{16b} are taken together with their intervening atom to form an optionally substituted, monocyclic, heterocyclic ring. In some embodiments, R^{16a} and R^{16b} are taken together with their intervening atom to form an optionally substituted, monocyclic, heterocyclic ring comprising O and/or S heteroatom(s) as the only heteroatoms in the heterocyclic ring. In some embodiments, R^{16a} and R^{16b} are taken together with their intervening atom to form an optionally substituted, monocyclic, heterocyclic ring comprising O and/or N heteroatom(s) as the only heteroatoms in the heterocyclic ring. In some embodiments, R^{16a} and R^{16b} are taken together with their intervening atom to form an optionally substituted, monocyclic, 3- to 14-membered heterocyclic ring. In some embodiments, R^{16a} and R^{16b} are taken together with their intervening atom to form an optionally substituted, monocyclic, 3- to 7-membered heterocyclic ring. In some embodiments, R^{16a} and R^{16b} are taken together with their intervening atom to form an optionally substituted, monocyclic, 5- to 6-membered heterocyclic ring.

In some embodiments, R^{16b} and R^{17a} are taken together with their intervening atom to form an optionally substituted, monocyclic, carbocyclic, heterocyclic, aryl, or heteroaryl ring. In some embodiments, R^{16b} and R^{17a} are taken together with their intervening atom to form an optionally substituted, monocyclic, carbocyclic or heterocyclic ring. In some embodiments, R^{16b} and R^{17a} are taken together with their intervening atom to form an optionally substituted, monocyclic, C₃₋₁₄ carbocyclic ring. In some embodiments, R^{16b} and R^{17a} are taken together with their intervening atom to form an optionally substituted, monocyclic, C₃₋₇ carbocyclic ring. In some embodiments, R^{16b} and R^{17a} are taken together with their intervening atom to form an optionally substituted, monocyclic, 3- to 14-membered heterocyclic ring. In some embodiments, R^{16b} and R^{17a} are taken together with their intervening atom to form an optionally substituted, monocyclic, 3- to 7-membered heterocyclic ring. In some embodiments, R^{16b} and R^{17a} are taken together with their intervening atom to form an optionally substituted, monocyclic, aryl, or heteroaryl ring. In some embodiments, R^{16b} and R^{17a} are taken together with their intervening atom to form an optionally substituted phenyl ring. In some embodiments, R^{16b} and R^{17a} are taken together with their intervening atom to form an optionally substituted, monocyclic, 5- or 6-membered heteroaryl ring.

In some embodiments, Formula II-A or II-B is any one of the formulae shown in Table 3:

TABLE 3

| | |
|---|---|
| 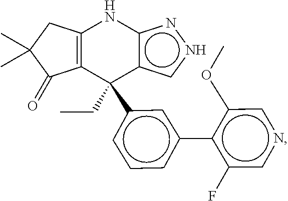 | 952562 |
| 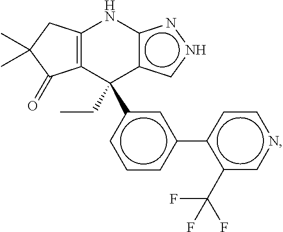 | 952757 |
| 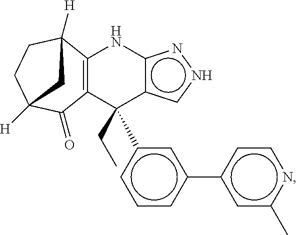 | 943921 |
| 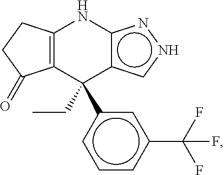 | 951566 |
| 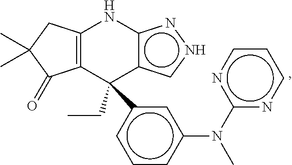 | 952205 |
| 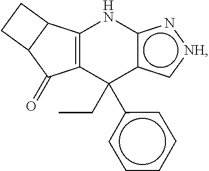 | 861716 |
| 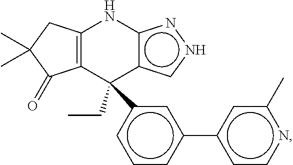 | 950283 |
| 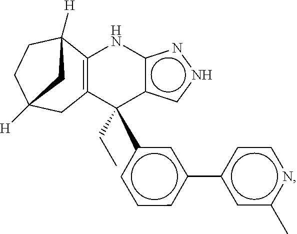 | 943920 |
| 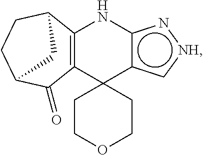 | 912081 |
| 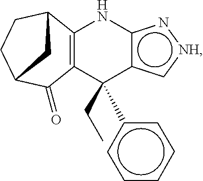 | 791270 |
| 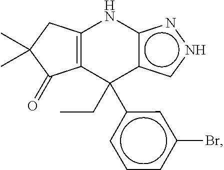 | 949805 |
| 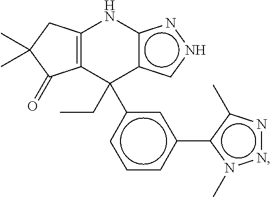 | 950815 |
| 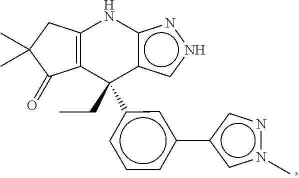 | 950819 |
| 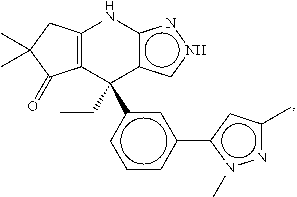 | 950818 |
| 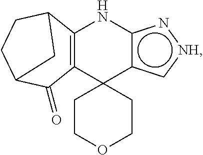 | 901464 |
| 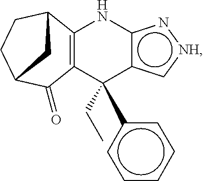 | 791269 |
| 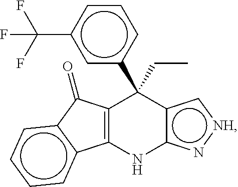 | 949742 |
| 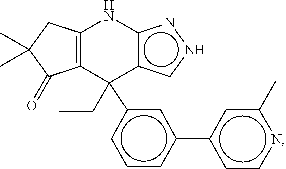 | 949840 |
| 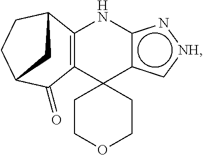 | 912080 |
| 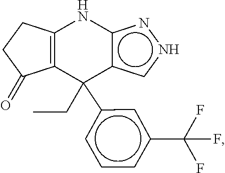 | 949639 |
| 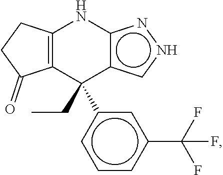 | 951565 |
| 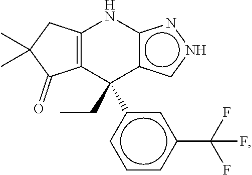 | 950272 |
| 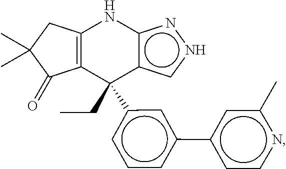 | 950282 |
| 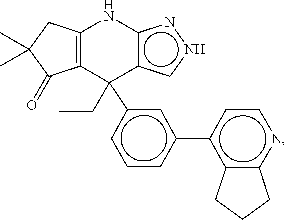 | 952557 |
| 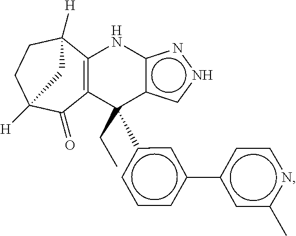 | 943917 |
| 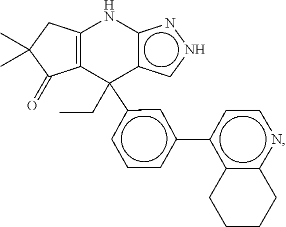 | 952752 |
| 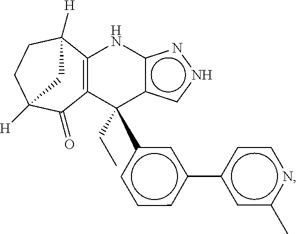 | 943916 |
| 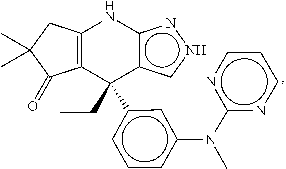 | 952212 |
| 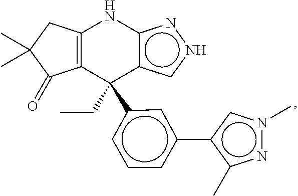 | 951702 |
| 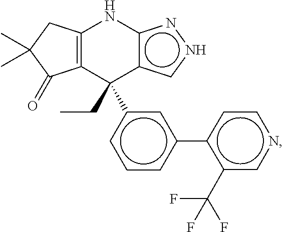 | 952759 |
| 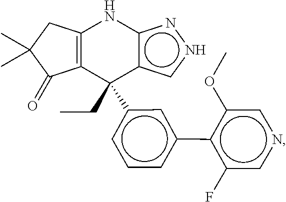 | 952564 |
| 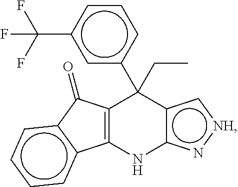 | 948205 |
| 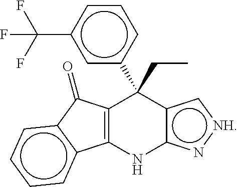 | 949743 |

In some embodiments, Formula II-A or II-B is any one of the formulae shown in Table 4:

TABLE 4

| | |
|---|---|
| 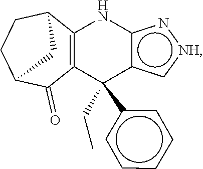 | 791266 |
| 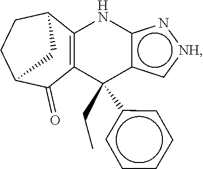 | 791268 |
| 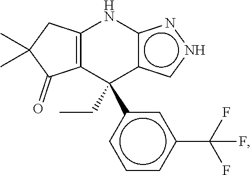 | 950273 |
| 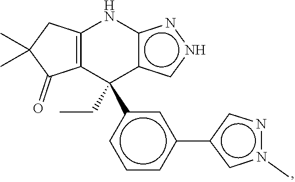 | 950826 |
| 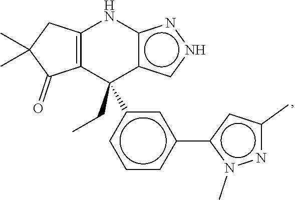 | 950827 |
| 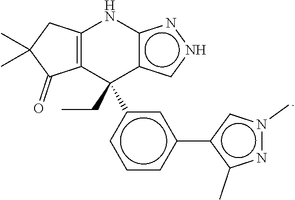 | 951710 |

In some embodiments, Formula II-A or II-B is not any one of the formulae shown in Table 4.

In another aspect, provided herein is a compound, or a pharmaceutically acceptable salt, solvate, hydrate, polymorph, co-crystal, tautomer, stereoisomer, isotopically labeled compound, or prodrug thereof, wherein the compound is of Formula III-A or III-B:

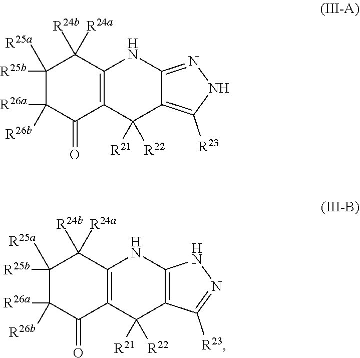

wherein:
R²¹ and R²² are taken together with their intervening atom to form a substituted monocyclic carbocyclic ring or an optionally substituted, monocyclic heterocyclic ring;
optionally two substituents on the monocyclic carbocyclic ring or monocyclic heterocyclic ring are taken together with their intervening atom(s) to form an additional optionally substituted, monocyclic, carbocyclic or heterocyclic ring;
R²³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{C}, —SCN, —SR^{C}, —SSR^{C}, —N₃, —NO, —N(R^{C})₂, —NO₂, —C(═O)R^{C}, —C(═O)OR^{C}, —C(═O)SR^{C}, —C(═O)N(R^{C})₂, —C(═NR^{C})R^{C}, —C(═NR^{C})OR^{C}, —C(═NR^{C})SR^{C}, —C(═NR^{C})N(R^{C})₂, —S(═O)R^{C}, —S(═O)OR^{C}, —S(═O)SR^{C}, —S(═O)N(R^{C})₂, —S(═O)₂R^{C}, —S(═O)OR^{C}, —S(═O)₂SR^{C}, —S(═O)₂N(R^{C})₂, —OC(═O)R^{C}, —OC(═O)OR^{C}, —OC(═O)SR^{C}, —OC(═O)N(R^{C})₂, —OC(═NR^{C})R^{C}, —OC(═NR^{C})OR^{C}, —OC(═NR^{C})SR^{C}, —OC(═NR^{C})N(R^{C})₂, —OS(—O)R^{C}, —OS(O)OR^{C}, —OS(═O)SR^{C}, —OS(═O)N(R^{C})₂, —OS(═O)₂R^{C}, —OS(═O)₂OR^{C}, —OS(═O)₂SR^{C}, —OS(═O)₂N(R^{C})₂, —ON(R^{C})₂, —SC(═O)R^{C}, —SC(═O)OR^{C}, —SC(═O)SR^{C}, —SC(═O)N(R^{C})₂, —SC(═NR^{C})R^{C}, —SC(═NR^{C})OR^{C}, —SC(═NR^{C})SR^{C}, —SC(═NR^{C})N(R^{C})₂, —NR^{C}C(═O)R^{C}, —NR^{C}C(═O)OR^{C}, —NR^{C}C(═O)SR^{C}, —NR^{C}C(═O)N(R^{C})₂, —NR^{C}C(═NR^{C})R^{C}, —NR^{C}C(═NRS)OR^{C}, —NR^{C}C(═NR^{C})SR^{C}, —NR^{C}C(═NR^{C})N(R^{C})₂, —NR^{C}S(═O)R^{C}, —NR^{C}S(═O)OR^{C}, —NR^{C}S(═O)SR^{C}, —NR^{C}S(═O)N(R^{C})₂, —NR^{C}S(═O)₂R^{C}, —NR^{C}S(═O)₂OR^{C}, —NR^{C}S(═O)₂SR^{C}, —NR^{C}S(═O)₂N(R^{C})₂, —Si(R^{C})₃, —Si(R^{C})₂OR^{C}, —Si(R^{C})(OR^{C})₂, —Si(OR^{C})₃, —OSi(R^{C})₃, —OSi(R^{C})₂OR^{C}, —OSi(R^{C})(OR^{C})₂, or —OSi(OR^{C})₃;
each of R^{24a}, R^{24b}, R^{25a}, R^{25b}, R^{26a}, and R^{26b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl; and
each instance of R^{C} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{C} attached to the same intervening atom are joined together with the intervening atom to form an optionally substituted, monocyclic, heterocyclic or heteroaryl ring;
or R^{24a} and R^{24b} are taken together with their intervening atom to form an optionally substituted, monocyclic, carbocyclic or heterocyclic ring;
or R^{24b} and R^{25a} are taken together with their intervening atom to form an optionally substituted, monocyclic, carbocyclic or heterocyclic ring;
or R^{25a} and R^{25b} are taken together with their intervening atom to form an optionally substituted, monocyclic, carbocyclic or heterocyclic ring;
or R^{25b} and R^{26a} are taken together with their intervening atom to form an optionally substituted, monocyclic, carbocyclic or heterocyclic ring;
or R^{26a} and R^{26b} are taken together with their intervening atom to form an optionally substituted, monocyclic, carbocyclic or heterocyclic ring;
provided that the compound is not of the formula:

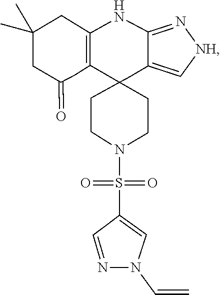 [SMILES: C=CN1C=C(S(=O)(=O)N2CCC3(CC2)Cc2c[nH]nc2NC2=C3CC(=O)CC(C)(C)C2)CC=N1]

or a pharmaceutically acceptable salt, solvate, hydrate, polymorph, co-crystal, tautomer, stereoisomer, isotopically labeled compound, or prodrug thereof.

In some embodiments, the compound is of the formula:

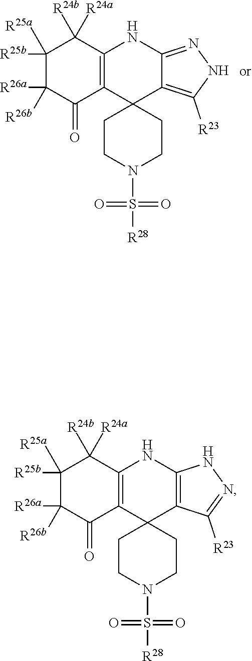

or a pharmaceutically acceptable salt, solvate, hydrate, polymorph, co-crystal, tautomer, stereoisomer, isotopically labeled compound, or prodrug thereof,
wherein:
R²⁸ is optionally substituted aryl or optionally substituted heteroaryl.

In some embodiments, the compound is of the formula:

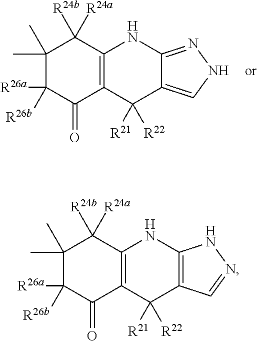

or a pharmaceutically acceptable salt, solvate, hydrate, polymorph, co-crystal, tautomer, stereoisomer, isotopically labeled compound, or prodrug thereof,
wherein:
R²¹ and R²² are taken together with their intervening atom to form an optionally substituted, monocyclic 5- to 7-membered N-heterocyclic ring.

In some embodiments, the compound is of the formula:

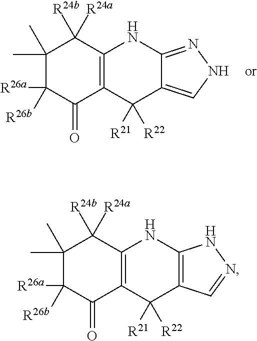

or a pharmaceutically acceptable salt, solvate, hydrate, polymorph, co-crystal, tautomer, stereoisomer, isotopically labeled compound, or prodrug thereof,
wherein:
R²¹ and R²² are taken together with their intervening atom to form a substituted monocyclic 4- to 6-membered carbocyclic ring. In some embodiments,

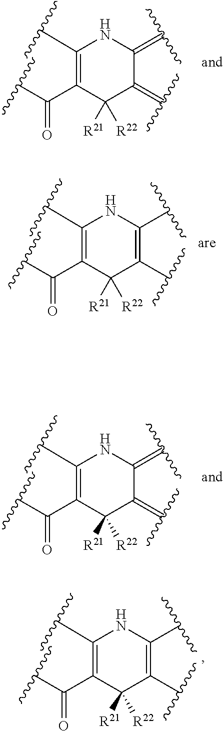

respectively. In some embodiments,

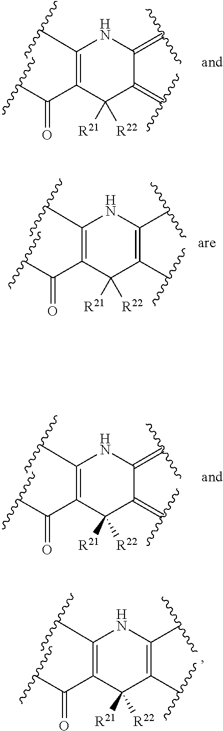

respectively.

In some embodiments, R²¹ and R²² are taken together with their intervening atom to form a substituted monocyclic carbocyclic ring, wherein optionally two substituents on the monocyclic carbocyclic ring are taken together with their intervening atom(s) to form an additional optionally substituted, monocyclic, carbocyclic or heterocyclic ring. In some embodiments, R²¹ and R²² are taken together with their intervening atom to form a substituted monocyclic carbocyclic ring. In some embodiments, R²¹ and R²² are taken together with their intervening atom to form a substituted monocyclic C₄₋₇ carbocyclic ring.

In some embodiments, R²¹ and R²² are taken together with their intervening atom to form a monocyclic carbocylic ring substituted with at least one instance of halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{C}, —SCN, —SR^{C}, —SSR^{C}, —N₃, —NO, —N(R^{C})₂, —NO₂, —C(═O)R^{C}, —C(═O)OR^{C}, —C(═O)SR^{C}, —C(═O)N(R^{C})₂, —C(═NR^{C})R^{C}, —C(═NR^{C})OR^{C}, —C(═NR^{C})SR^{C}, —C(NR^{C})N(R^{C})₂, —S(O)R^{C}, —S(═O)OR^{C}, —S(═O)SR^{C}, —S(═O)N(R^{C})₂, —S(═O)₂R^{C}, —S(═O)₂OR^{C}, —S(═O)₂SR^{C}, —S(═O)₂N(R^{C})₂, —OC(═O)R^{C}, —OC(═O)OR^{C}, —OC(═O)SR^{C}, —OC(═O)N(R^{C})₂, —OC(═NR^{C})R^{C}, —OC(NR^{C})OR^{C}, —OC(NR^{C})SR^{C}, —OC(═NR^{C})N(R^{C})₂, —OS(═O)R^{C}, —OS(═O)OR^{C}, —OS(═O)SR^{C}, —OS(═O)N(R^{C})₂, —OS(═O)₂R^{C}, —OS(═O)₂OR^{C}, —OS(═O)SR^{C}, —OS(═O)₂N(R^{C})₂, —ON(R^{C})₂, —SC(═O)R^{C}, —SC(═O)OR^{C}, —SC(═O)SR^{C}, —SC(═O)N(R^{C})₂, —SC(NR^{C})R^{C}, —SC(═NR^{C})OR^{C}, —SC(═NR^{C})SR^{C}, —SC(═NR^{C})N(R^{C})₂, —NR^{C}C(═O)R^{C}, —NR^{C}C(═O)OR^{C}, —NR^{C}C(═O)SR^{C}, —NR^{C}C(═O)N(R^{C})₂, —NR^{C}C(═NR^{C})R^{C}, —NR^{C}C(═NR^{C})OR^{C}, —NR^{C}C(═NR^{C})SR^{C}, —NR^{C}C(═NR^{C})N(R^{C})₂, —NR^{C}S(═O)R^{C}, —NR^{C}S(═O)OR^{C}, —NR^{C}S(═O)SR^{C}, —NR^{C}S(═O)N(R^{C})₂, —NR^{C}S(═O)₂R^{C}, —NR^{C}S(═O)₂OR^{C}, —NR^{C}S(═O)₂SR^{C}, —NR^{C}S(═O)N(R^{C})₂, —Si(R^{C})₃, —Si(R^{C})₂OR^{C}, —Si(R^{C})(OR^{C})₂, —Si(OR^{C})₃, —OSi(R^{C})₃, —OSi(R^{C})₂OR^{C}, —OSi(R^{C})(OR^{C})₂, or —OSi(OR^{C})₃.

In some embodiments, R²¹ and R²² are taken together with their intervening atom to form a monocyclic carbocylic ring substituted with at least one instance of halogen, optionally substituted alkyl, optionally substituted alkenyl, optionally substituted alkynyl, optionally substituted heteroalkyl, optionally substituted heteroalkenyl, optionally substituted heteroalkynyl, —OR^{C}, —SR^{C}, or —N(R^{C})₂. In some embodiments, R²¹ and R²² are taken together with their intervening atom to form a monocyclic carbocylic ring substituted with at least one instance of optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R²¹ and R²² are taken together with their intervening atom to form a monocyclic carbocylic ring substituted with at least one instance of C₁₋₁₀ haloalkyl. In some embodiments, R²¹ and R²² are taken together with their intervening atom to form a monocyclic carbocylic ring substituted with at least one instance of C₁₋₄ haloalkyl. In some embodiments, R²¹ and R²² are taken together with their intervening atom to form a monocyclic carbocylic ring substituted with at least one instance of C₁₋₄ fluoroalkyl (e.g., C₁₋₄ perfluoroalkyl). In some embodiments, R²¹ and R²² are taken together with their intervening atom to form a monocyclic carbocylic ring substituted with at least one instance of —CF₃. In some embodiments, R²¹ and R²² are taken together with their intervening atom to form a monocyclic carbocylic ring substituted with at least one instance of optionally substituted C₁-C₆ alkyl or halogen. In some embodiments, R²¹ and R²² are taken together with their intervening atom to form a monocyclic carbocylic ring substituted with at least one instance of fluorine, —CH₃, or —CF₃.

In some embodiments, R²¹ and R²² are taken together with their intervening atom to form a monocyclic carbocylic ring substituted with at least one instance of halogen. In some embodiments, R²¹ and R²² are taken together with their intervening atom to form a monocyclic carbocylic ring substituted with at least one instance of bromine, chlorine, or fluorine. In some embodiments, R²¹ and R²² are taken together with their intervening atom to form a monocyclic carbocylic ring substituted with at least one instance of bromine or chlorine. In some embodiments, R²¹ and R²² are taken together with their intervening atom to form a monocyclic carbocylic ring substituted with at least one instance of chlorine or fluorine. In some embodiments, R²¹ and R²² are taken together with their intervening atom to form a monocyclic carbocylic ring substituted with at least one instance of bromine. In some embodiments, R²¹ and R²² are taken together with their intervening atom to form a monocyclic carbocylic ring substituted with at least one instance of chlorine. In some embodiments, R²¹ and R²² are taken together with their intervening atom to form a monocyclic carbocylic ring substituted with at least one instance of fluorine.

In some embodiments, R²¹ and R²² are taken together with their intervening atom to form a monocyclic carbocylic ring substituted with at least one instance of —OR^{C}, —SR^{C}, or —N(R^{C})₂ (e.g., wherein R^{C} is hydrogen or optionally substituted alkyl, or optionally substituted phenyl). In some embodiments, R²¹ and R²² are taken together with their intervening atom to form a monocyclic carbocylic ring substituted with at least one instance of —OH. In some embodiments, R²¹ and R²² are taken together with their intervening atom to form a monocyclic carbocylic ring substituted with at least one instance of —SH. In some embodiments, R²¹ and R²² are taken together with their intervening atom to form a monocyclic carbocylic ring substituted with at least one instance of —NH₂. In some embodiments, R²¹ and R²² are taken together with their intervening atom to form a monocyclic carbocylic ring substituted with at least one instance of —CN, —SCN, —SSR^{C}, —N₃, —NO, or —NO₂. In some embodiments, R²¹ and R²² are taken together with their intervening atom to form a monocyclic carbocylic ring substituted with at least one instance of —C(═O)R^{C}, —C(═O)OR^{C}, —C(═O)SR^{C}, or —C(═O)N(R^{C})₂ (e.g., wherein R^{C} is hydrogen or optionally substituted alkyl, or optionally substituted phenyl). In some embodiments, R²¹ and R²² are taken together with their intervening atom to form a monocyclic carbocylic ring substituted with at least one instance of —C(═NR^{C})OR^{C}, —C(NR^{C})OR^{C}, —C(NR^{C})SR^{C}, or —C(═NR^{C})N(R^{C})₂ (e.g., wherein R^{C} is hydrogen or optionally substituted alkyl, or optionally substituted phenyl). In some embodiments, R²¹ and R²² are taken together with their intervening atom to form a monocyclic carbocylic ring substituted with at least one instance of —S(═O)R^{C}, —S(═O)OR^{C}, —S(═O)SR^{C}, —S(═O)N(R^{C})₂, —S(═O)₂R^{C}, —S(═O)OR^{C}, —S(═O)SR^{C}, or —S(═O)₂N(R^{C})₂ (e.g., wherein R^{C} is hydrogen or optionally substituted alkyl, or optionally substituted phenyl). In some embodiments, R²¹ and R²² are taken together with their intervening atom to form a monocyclic carbocylic ring substituted with at least one instance of —OC(═O)R^{C}, —OC(═O)OR^{C}, —OC(═O)SR^{C}, —OC(═O)N(R^{C})₂, —OC(═NR^{C})R^{C}, —OC(═NR^{C})OR^{C}, —OC(═NR^{C})SR^{C}, —OC(═NR^{C})N(R^{C})₂, —OS(═O)R^{C}, —OS(═O)OR^{C}, —OS(═O)SR^{C}, —OS(═O)N(R^{C})₂, —OS(═O)₂R^{C}, —OS(═O)₂OR^{C}, —OS(═O)₂SR^{C}, —OS(═O)₂N(R^{C})₂, or —ON(R^{C})₂ (e.g., wherein R^{C} is hydrogen or optionally substituted alkyl, or optionally substituted phenyl). In some embodiments, R²¹ and R²² are taken together with their intervening atom to form a monocyclic carbocylic ring substituted with at least one instance of —SC(═O)R^{C}, —SC(═O)OR^{C}, —SC(═O)SR^{C}, —SC(═O)N(R⁹)₂, —SC(═NR^{C})R^{C}, —SC(═NR^{C})OR^{C}, —SC(═NR^{C})SR^{C}, or —SC(═NR^{C})N(R^{C})₂ (e.g., wherein R^{C} is hydrogen or optionally substituted alkyl, or optionally substituted phenyl). In some embodiments, R²¹ and R²² are taken together with their intervening atom to form a monocyclic carbocylic ring substituted with at least one instance of —NR^{C}C(═O)R^{C}, —NR^{C}C(═O)OR^{C}, —NR^{C}C(═NR^{C})N(R^{C})₂, —NR^{C}S(═O)R^{C}, —NR^{C}S(═O)OR^{C}, —NR^{C}S(═O)SR^{C}, —NR^{C}S(═O)N(R^{C})₂, —NR^{C}S(═O)₂R^{C}, —NR^{C}S(═O)₂OR^{C}, —NR^{C}S(═O)₂SR^{C}, or —NR^{C}S(═O)₂N(R^{C})₂ (e.g., wherein R^{C} is hydrogen or optionally substituted alkyl, or optionally substituted phenyl). In some embodiments, R²¹ and R²² are taken together with their intervening atom to form a monocyclic carbocylic ring substituted with at least one instance of —Si(R^{C}), —Si(R^{C})₂OR^{C}, —Si(R^{C})(OR^{C})₂, —Si(OR^{C})₃, —OSi(R^{C})₃, —OSi(R^{C})₂OR^{C}, —OSi(R^{C})(OR^{C})₂, or —OSi(OR^{C})₃ (e.g., wherein R^{C} is hydrogen or optionally substituted alkyl, or optionally substituted phenyl).

In some embodiments, R²¹ and R²² are taken together with their intervening atom to form a monocyclic carbocylic ring substituted with at least one instance of optionally substituted carbocyclyl, optionally substituted heterocyclyl, optionally substituted aryl, or optionally substituted heteroaryl. In some embodiments, R²¹ and R²² are taken together with their intervening atom to form a monocyclic carbocylic ring substituted with at least one instance of optionally substituted C₃₋₁₄ carbocyclyl. In some embodiments, R²¹ and R²² are taken together with their intervening atom to form a monocyclic carbocylic ring substituted with at least one instance of optionally substituted monocyclic C₃₋₇ carbocyclyl. In some embodiments, R²¹ and R²² are taken together with their intervening atom to form a monocyclic carbocylic ring substituted with at least one instance of optionally substituted monocyclic C₃₋₄ carbocyclyl. In some embodiments, R²¹ and R²² are taken together with their intervening atom to form a monocyclic carbocylic ring substituted with at least one instance of optionally substituted monocyclic C₅₋₇ carbocyclyl. In some embodiments, R²¹ and R²² are taken together with their intervening atom to form a monocyclic carbocylic ring substituted with at least one instance of saturated carbocyclyl. In some embodiments, R²¹ and R²² are taken together with their intervening atom to form a monocyclic carbocylic ring substituted with at least one instance of carbocyclyl comprising only one unsaturated bond (e.g., C═C bond) in the carbocyclic ring system.

In some embodiments, R²¹ and R²² are taken together with their intervening atom to form a monocyclic carbocylic ring substituted with at least one instance of optionally substituted 3- to 14-membered heterocyclyl. In some embodiments, R²¹ and R²² are taken together with their intervening atom to form a monocyclic carbocylic ring substituted with at least one instance of optionally substituted monocyclic 3- to 7-membered heterocyclyl. In some embodiments, R²¹ and R²² are taken together with their intervening atom to form a monocyclic carbocylic ring substituted with at least one instance of optionally substituted 3- to 14-membered heterocyclyl comprising at least one O and/or S atoms but no N atoms. In some embodiments, R²¹ and R²² are taken together with their intervening atom to form a monocyclic carbocylic ring substituted with at least one instance of optionally substituted 3- to 14-membered heterocyclyl comprising at least one N atoms and optionally at least one O and/S atoms. In some embodiments, R²¹ and R²² are taken together with their intervening atom to form a monocyclic carbocylic ring substituted with at least one instance of optionally substituted monocyclic 3- to 7-membered heterocyclyl comprising at least one O and/or S atoms but no N atoms. In some embodiments, R²¹ and R²² are taken together with their intervening atom to form a monocyclic carbocylic ring substituted with at least one instance of optionally substituted monocyclic 3- to 7-membered heterocyclyl comprising at least one N atoms and optionally at least one O and/or S atoms. In some embodiments, R²¹ and R²² are taken together with their intervening atom to form a monocyclic carbocylic ring substituted with at least one instance of saturated heterocyclyl. In some embodiments, R²¹ and R²² are taken together with their intervening atom to form a monocyclic carbocylic ring substituted with at least one instance of heterocyclyl comprising only one unsaturated bond (e.g., C═C bond) in the heterocyclic ring system.

In some embodiments, R²¹ and R²² are taken together with their intervening atom to form a monocyclic carbocylic ring substituted with at least one instance of optionally substituted monocyclic aryl. In some embodiments, R²¹ and R²² are taken together with their intervening atom to form a monocyclic carbocylic ring substituted with at least one instance of optionally substituted bicyclic aryl. In some embodiments, R²¹ and R²² are taken together with their intervening atom to form a monocyclic carbocylic ring substituted with at least one instance of optionally substituted C₆₋₁₄ aryl. In some embodiments, R²¹ and R²² are taken together with their intervening atom to form a monocyclic carbocylic ring substituted with at least one instance of optionally substituted C₆₋₁₀ aryl. In some embodiments, R²¹ and R²² are taken together with their intervening atom to form a monocyclic carbocylic ring substituted with at least one instance of optionally substituted phenyl. In some embodiments, R²¹ and R²² are taken together with their intervening atom to form a monocyclic carbocylic ring substituted with at least one instance of optionally substituted naphthyl.

In some embodiments, R²¹ and R²² are taken together with their intervening atom to form a monocyclic carbocylic ring substituted with at least one instance of optionally substituted monocyclic heteroaryl. In some embodiments, R²¹ and R²² are taken together with their intervening atom to form a monocyclic carbocylic ring substituted with at least one instance of optionally substituted bicyclic heteroaryl. In some embodiments, R²¹ and R²² are taken together with their intervening atom to form a monocyclic carbocylic ring substituted with at least one instance of optionally substituted 5- to 14-membered heteroaryl. In some embodiments, R²¹ and R²² are taken together with their intervening atom to form a monocyclic carbocylic ring substituted with at least one instance of optionally substituted 5- to 10-membered heteroaryl. In some embodiments, R²¹ and R²² are taken together with their intervening atom to form a monocyclic carbocylic ring substituted with at least one instance of optionally substituted 5- to 6-membered monocyclic heteroaryl.

In some embodiments, R²¹ and R²² are taken together with their intervening atom to form a monocyclic carbocyclic ring substituted with at least one instances of halogen, —OMe, —OBn, —CN, optionally substituted C₁₋₄ alkyl, optionally substituted aryl, optionally substituted heteroaryl, —C(═O) NHMe, —C(═O) NMe₂, —C(═O)OMe, ═O, or —S(═O)₂Me.

In some embodiments, R²¹ and R²² are taken together with their intervening atom to form a substituted monocyclic carbocyclic ring, wherein two substituents on the monocyclic carbocyclic ring are taken together with their intervening atom(s) to form an additional optionally substituted, monocyclic, carbocyclic or heterocyclic ring. In some embodiments, R²¹ and R²² are taken together with their intervening atom to form a substituted monocyclic carbocyclic ring, wherein two substituents on the monocyclic carbocyclic ring are taken together with their intervening atom(s) to form an additional optionally substituted, monocyclic, carbocyclic ring. In some embodiments, R²¹ and R²² are taken together with their intervening atom to form a substituted monocyclic carbocyclic ring, wherein two substituents on the monocyclic carbocyclic ring are taken together with their intervening atom(s) to form an additional optionally substituted, monocyclic, C₃₋₇ carbocyclic ring. In some embodiments, R²¹ and R²² are taken together with their intervening atom to form a substituted monocyclic carbocyclic ring, wherein two substituents on the monocyclic carbocyclic ring are taken together with their intervening atom(s) to form an additional optionally substituted, monocyclic, C₃₋₄ carbocyclic ring. In some embodiments, R²¹ and R²² are taken together with their intervening atom to form a substituted monocyclic carbocyclic ring, wherein two substituents on the monocyclic carbocyclic ring are taken together with their intervening atom(s) to form an additional optionally substituted, monocyclic, C₅₋₇ carbocyclic ring.

In some embodiments, R²¹ and R²² are taken together with their intervening atom to form a substituted monocyclic carbocyclic ring, wherein two substituents on the monocyclic carbocyclic ring are taken together with their intervening atom(s) to form an additional optionally substituted, monocyclic, heterocyclic ring. In some embodiments, R²¹ and R²² are taken together with their intervening atom to form a substituted monocyclic carbocyclic ring, wherein two substituents on the monocyclic carbocyclic ring are taken together with their intervening atom(s) to form an additional optionally substituted, monocyclic, 3- to 7-membered heterocyclic ring. In some embodiments, R²¹ and R²² are taken together with their intervening atom to form a substituted monocyclic carbocyclic ring, wherein two substituents on the monocyclic carbocyclic ring are taken together with their intervening atom(s) to form an additional optionally substituted, monocyclic, 3- to 4-membered heterocyclic ring. In some embodiments, R²¹ and R²² are taken together with their intervening atom to form a substituted monocyclic carbocyclic ring, wherein two substituents on the monocyclic carbocyclic ring are taken together with their intervening atom(s) to form an additional optionally substituted, monocyclic, 5- to 7-membered heterocyclic ring.

In some embodiments, R²¹ and R²² are taken together with their intervening atom to form an optionally substituted monocyclic heterocyclic ring, wherein optionally two substituents on the monocyclic heterocyclic ring are taken together with their intervening atom(s) to form an additional optionally substituted, monocyclic, carbocyclic or heterocyclic ring. In some embodiments, R²¹ and R²² are taken together with their intervening atom to form an optionally substituted monocyclic heterocyclic ring. In some embodiments, R²¹ and R²² are taken together with their intervening atom to form an optionally substituted, 4- to 7-membered monocyclic heterocyclic ring. In some embodiments, R²¹ and R²² are taken together with their intervening atom to form an optionally substituted, 5- to 6-membered monocyclic heterocyclic ring. In some embodiments, the monocyclic heterocyclic ring comprises O and/or N heteroatom(s) as the only heteroatoms in the heterocyclic ring. In some embodiments, R²¹ and R²² are taken together with their intervening atom to form optionally substituted tetrahydropyranyl. In some embodiments, R²¹ and R²² are taken together with their intervening atom to form an optionally substituted piperidine ring. In some embodiments, R²¹ and R²² are taken together with their intervening atom to form

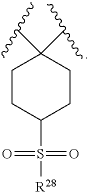

R²⁸ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a sulfur protecting group. In some embodiments, R²⁸ is hydrogen, optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, optionally substituted C₁₋₁₀ heteroalkynyl, optionally substituted C₃₋₁₄ carbocyclyl, optionally substituted 3- to 14-membered heterocyclyl, optionally substituted C₆₋₁₄ aryl, optionally substituted 5-14-membered heteroaryl, or a sulfur protecting group.

In some embodiments, R²¹ and R²² are taken together with their intervening atom to form an optionally substituted monocyclic heterocyclic ring, wherein two substituents on the monocyclic heterocyclic ring are taken together with their intervening atom(s) to form an additional optionally substituted, monocyclic, carbocyclic or heterocyclic ring. In some embodiments, R²¹ and R²² are taken together with their intervening atom to form an optionally substituted monocyclic heterocyclic ring, wherein two substituents on the monocyclic heterocyclic ring are taken together with their intervening atom(s) to form an additional optionally substituted, monocyclic, carbocyclic ring. In some embodiments, R²¹ and R²² are taken together with their intervening atom to form an optionally substituted monocyclic heterocyclic ring, wherein two substituents on the monocyclic heterocyclic ring are taken together with their intervening atom(s) to form an additional optionally substituted, monocyclic, heterocyclic ring. In some embodiments, two substituents on the monocyclic heterocyclic ring are taken together with their intervening atom(s) to form an additional 4- to 7-membered optionally substituted, monocyclic, carbocyclic or heterocyclic ring. In some embodiments, R²¹ and R²² are taken together with their intervening atom to form

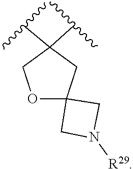

R²⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group. In some embodiments, R²⁹ is hydrogen, optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, optionally substituted C₁₋₁₀ heteroalkynyl, optionally substituted C₃₋₁₄ carbocyclyl, optionally substituted 3- to 14-membered heterocyclyl, optionally substituted C₆₋₁₄ aryl, optionally substituted 5-14-membered heteroaryl, or a nitrogen protecting group.

In some embodiments, R²³ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{C}, —SR^{C}, or —N(R^{C})₂. In some embodiments, R²³ is optionally substituted C₁₋₁₀ alkyl, optionally substituted C₁₋₁₀ alkenyl, substituted C₁₋₁₀ heteroalkenyl, or optionally substituted C₁₋₁₀ heteroalkynyl. In some embodiments, R²³ is C₁₋₁₀ haloalkyl. In some embodiments, R²³ is C₁₋₄ haloalkyl. In some embodiments, R²³ is C₁₋₄ fluoroalkyl (e.g., C₁₋₄ perfluoroalkyl). In some embodiments, R²³ is —CF₃. In some embodiments, R²³ is hydrogen, optionally substituted C₁-C₆ alkyl, or halogen. In some embodiments, R²³ is hydrogen, fluorine, —CH₃, —CH₂F, —CHF₂, or —CF₃. In some embodiments, R²³ is hydrogen, fluorine, —CH₃, or —CF₃. In some embodiments, R²³ is hydrogen or halogen. In some embodiments, R²³ is hydrogen or fluorine. In some embodiments, R²³ is hydrogen or optionally substituted C₁-C₆ alkyl. In some embodiments, R²³ is hydrogen, unsubstituted C₁-C₆ alkyl, or C₁₋₆ haloalkyl. In some embodiments, R²³ is hydrogen.

In some embodiments, R²³ is halogen. In some embodiments, R²³ is bromine, chlorine, or fluorine. In some embodiments, R²³ is bromine or chlorine. In some embodiments, R²³ is chlorine or fluorine. In some embodiments, R²³ is bromine. In some embodiments, R²³ is chlorine. In some embodiments, R²³ is fluorine.

In some embodiments, R²³ is —OR^{C}, —SR^{C}, or —N(R^{C})₂ (e.g., wherein R^{C} is hydrogen or optionally substituted alkyl, or optionally substituted phenyl). In some embodiments, R²³ is —OH. In some embodiments, R²³ is —SH. In some embodiments, R²³ is —NH₂. In some embodiments, R²³ is —CN, —SCN, —SSR^{C}, —N₃, —NO, or —NO₂. In some embodiments, R²³ is —C(═O)R^{C}, —C(═O)OR^{C}, —C(═O)SR^{C}, or —C(═O)N(R^{C})₂ (e.g., wherein R^{C} is hydrogen or optionally substituted alkyl, or optionally substituted phenyl). In some embodiments, R²³ is —C(═NR^{C})R^{C}, —C(═NR^{C})OR^{C}, —C(═NR^{C})SR^{C}, or —C(═NR^{C})N(R^{C})₂ (e.g., wherein R^{C} is hydrogen or optionally substituted alkyl, or optionally substituted phenyl). In some embodiments, R²³ is —S(═O)R^{C}, —S(═O)OR^{C}, —S(═O)SR^{C}, —S(═O)N(R^{C})₂, —S(═O)R^{C}, —S(═O)₂OR^{C}, —S(═O)₂SR^{C}, or —S(═O)₂N(R^{C})₂ (e.g., wherein R^{C} is hydrogen or optionally substituted alkyl, or optionally substituted phenyl). In some embodiments, R²³ is —OC(═O)R^{C}, —OC(═O)OR^{C}, —OC(═O)SR^{C}, —OC(═O)N(R^{C})₂, —OC(═NR^{C})R^{C}, —OC(═NR^{C})OR^{C}, —OC(═NR^{C})SR^{C}, —OC(═NR^{C})N(R^{C})₂, —OS(═O)R^{C}, —OS(═O)OR^{C}, —OS(═O)SR^{C}, —OS(═O)N(R^{C})₂, —OS(═O)₂R^{C}, —OS(═O)₂OR^{C}, —OS(═O)₂SR^{C}, —OS(═O)₂N(R^{C})₂, or —ON(R^{C})₂ (e.g., wherein R^{C} is hydrogen or optionally substituted alkyl, or optionally substituted phenyl). In some embodiments, R²³ is —SC(═O)R^{C}, —SC(═O)OR^{C}, —SC(═O)SR^{C}, —SC(═O)N(R^{C})₂, —SC(═NR^{C})R^{C}, —SC(═NR^{C})OR^{C}, —SC(═NR^{C})SR^{C}, or —SC(═NR^{C})N(R^{C})₂ (e.g., wherein R^{C} is hydrogen or optionally substituted alkyl, or optionally substituted phenyl). In some embodiments, R²³ is —NR^{C}C(═O)R^{C}, —NR^{C}C(═O)OR^{C}, —NR^{C}C(═O)SR^{C}, —NR^{C}C(═O)N(R^{C})₂, —NR^{C}C(═NR^{C})R^{C}, —NR^{C}C(═NR^{C})OR^{C}, —NR^{C}C(═NR^{C})SR^{C}, —NR^{C}C(═NR^{C})N(R^{C})₂, —NR^{C}S(═O)R^{C}, —NR^{C}S(═O)OR^{C}, —NR^{C}S(═O)SR^{C}, —NR^{C}S(═O)N(R^{C})₂, —NR^{C}S(═O)₂R^{C}, —NR^{C}S(═O)₂OR^{C}, —NR^{C}S(═O)₂SR^{C}, or —NR^{C}S(═O)₂N(R^{C})₂ (e.g., wherein R^{C} is hydrogen or optionally substituted alkyl, or optionally substituted phenyl). In some embodiments, R²³ is —Si(R^{C})₃, —Si(R^{C})₂OR^{C}, —Si(R^{C})(OR^{C})₂, —Si(OR^{C})₃, —OSi(R^{C})₃, —OSi(R^{C})₂OR^{C}, —OSi(R^{C})(OR^{C})₂, or —OSi(OR^{C})₃ (e.g., wherein R^{C} is hydrogen or optionally substituted alkyl, or optionally substituted phenyl).

In some embodiments, R²³ is optionally substituted carbocyclyl, optionally substituted heterocyclyl, optionally substituted aryl, or optionally substituted heteroaryl. In some embodiments, R²³ is optionally substituted C₃₋₁₄ carbocyclyl. In some embodiments, R²³ is optionally substituted monocyclic C₃₋₇ carbocyclyl. In some embodiments, R²³ is optionally substituted monocyclic C₃₋₄ carbocyclyl. In some embodiments, R²³ is optionally substituted monocyclic C₅₋₇ carbocyclyl. In some embodiments, R²³ is saturated carbocyclyl. In some embodiments, R²³ is carbocyclyl comprising only one unsaturated bond (e.g., C═C bond) in the carbocyclic ring system.

In some embodiments, R²³ is optionally substituted 3- to 14-membered heterocyclyl. In some embodiments, R²³ is optionally substituted monocyclic 3- to 7-membered heterocyclyl. In some embodiments, R²³ is optionally substituted 3- to 14-membered heterocyclyl comprising one or more O and/or S atoms but no N atoms. In some embodiments, R²³ is optionally substituted 3- to 14-membered heterocyclyl comprising one or more N atoms and optionally one or more O and/S atoms. In some embodiments, R²³ is optionally substituted monocyclic 3- to 7-membered heterocyclyl comprising one or more O and/or S atoms but no N atoms. In some embodiments, R²³ is optionally substituted monocyclic 3- to 7-membered heterocyclyl comprising one or more N atoms and optionally one or more O and/or S atoms. In some embodiments, R²³ is saturated heterocyclyl. In some embodiments, R²³ is heterocyclyl comprising only one unsaturated bond (e.g., C═C bond) in the heterocyclic ring system.

In some embodiments, R²³ is optionally substituted monocyclic aryl. In some embodiments, R²³ is optionally substituted bicyclic aryl. In some embodiments, R²³ is optionally substituted C₆₋₁₄ aryl. In some embodiments, R²³ is optionally substituted C₆₋₁₀ aryl. In some embodiments, R²³ is optionally substituted phenyl. In some embodiments, R²³ is optionally substituted naphthyl.

In some embodiments, R²³ is optionally substituted monocyclic heteroaryl. In some embodiments, R²³ is optionally substituted bicyclic heteroaryl. In some embodiments, R²³ is optionally substituted 5- to 14-membered heteroaryl. In some embodiments, R²³ is optionally substituted 5- to 10-membered heteroaryl. In some embodiments, R²³ is optionally substituted 5- to 6-membered monocyclic heteroaryl.

In some embodiments, at least one instance of R^{C} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{C} is optionally substituted carbocyclyl, optionally substituted heterocyclyl, optionally substituted aryl, or optionally substituted heteroaryl. In some embodiments, at least one instance of R^{C} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{C} is independently hydrogen, optionally substituted C₁₋₁₀ alkyl, or optionally substituted phenyl. In some embodiments, at least one instance of R^{C} is a nitrogen protecting group when attached to a nitrogen atom, an oxygen protecting group when attached to an oxygen atom, or a sulfur protecting group when attached to a sulfur atom. In some embodiments, two instances of R^{C} attached to the same intervening atom are joined together with the intervening atom to form an optionally substituted, monocyclic, heterocyclic or heteroaryl ring.

In some embodiments, each of R^{24a}, R^{24b}, R^{25a}, R^{25b}, R^{26a}, and R^{26b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, each of R^{24a}, R^{24b}, R^{25a}, R^{25b}, R^{26a}, and R^{26b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, each of R^{24a}, R^{24b}, R^{25a}, R^{25b}, R^{26a}, and R^{26b} is independently hydrogen, halogen, or optionally substituted alkyl. In some embodiments, each of R^{24a}, R^{24b}, R^{25a}, R^{25b}, R^{26a}, and R^{26b} is independently hydrogen, halogen, or optionally substituted C₁₋₄ alkyl. In some embodiments, each of R^{24a}, R^{24b}, R^{25a}, R^{25b}, R^{26a}, and R^{26b} is independently hydrogen, fluorine, or optionally substituted C₁₋₄ alkyl. In some embodiments, each of R^{24a}, R^{24b}, R^{25a}, R^{25b}, R^{26a}, and R^{26b} is independently hydrogen or halogen. In some embodiments, each of R^{24a} and R^{24b} is independently hydrogen or fluorine. In some embodiments, each of R^{24a}, R^{24b}, R^{25a}, R^{25b}, R^{26a}, and R^{26b} is independently hydrogen or optionally substituted C₁₋₄ alkyl. In some embodiments, at least one of R^{24a}, R^{24b}, R^{25a}, R^{25b}, R^{26a}, and R^{26b} is hydrogen. In some embodiments, each of R^{24a}, R^{24b}, R^{25a}, R^{25b}, and R^{26b} is hydrogen. In some embodiments, at least one of R^{24a}, R^{24b}, R^{25a}, R^{25b}, R^{26a}, and R^{26b} is optionally substituted C₁₋₄ alkyl. In some embodiments, at least one of R^{24a}, R^{24b}, R^{25a}, R^{25b}, R^{26a}, and R^{26b} is unsubstituted methyl. In some embodiments, at least one of R^{24a}, R^{24b}, R^{25a}, R^{25b}, R^{26a}, and R^{26b} is fluorine.

In some embodiments, each of R^{24a} and R^{24b} is independently hydrogen, halogen, or optionally substituted alkyl. In some embodiments, at least one of R^{24a} and R^{24b} is fluorine.

In some embodiments, each of R^{25a} and R^{25b} is independently hydrogen, halogen, optionally substituted alkyl, or optionally substituted aryl. In some embodiments, R^{25a} and R^{25b} are —CH₃.

In some embodiments, each of R^{26a} and R^{26b} is independently hydrogen, halogen, or optionally substituted alkyl. In some embodiments, at least one of R^{26a} and R^{26b} is fluorine.

In some embodiments, R^{24a} and R^{24b} are taken together with their intervening atom to form an optionally substituted, monocyclic, carbocyclic or heterocyclic ring. In some embodiments, R^{24a} and R^{24b} are taken together with their intervening atom to form an optionally substituted, monocyclic, C₃₋₁₄ carbocyclic ring. In some embodiments, R^{24a} and R^{24b} are taken together with their intervening atom to form an optionally substituted, monocyclic, C₃₋₇ carbocyclic ring. In some embodiments, R^{24a} and R^{24b} are taken together with their intervening atom to form an optionally substituted, monocyclic, 3- to 14-membered heterocyclic ring. In some embodiments, R^{24a} and R^{24b} are taken together with their intervening atom to form an optionally substituted, monocyclic, 3- to 7-membered heterocyclic ring.

In some embodiments, R^{24b} and R^{25a} are taken together with their intervening atom to form an optionally substituted, monocyclic, carbocyclic or heterocyclic ring. In some embodiments, R^{24b} and R^{25a} are taken together with their intervening atom to form an optionally substituted, monocyclic, C₃₋₁₄ carbocyclic ring. In some embodiments, R^{24b} and R^{25a} are taken together with their intervening atom to form an optionally substituted, monocyclic, C₃₋₇ carbocyclic ring. In some embodiments, R^{24b} and R^{25a} are taken together with their intervening atom to form an optionally substituted, monocyclic, 3- to 14-membered heterocyclic ring. In some embodiments, R^{24b} and R^{25a} are taken together with their intervening atom to form an optionally substituted, monocyclic, 3- to 7-membered heterocyclic ring.

In some embodiments, R^{25a} and R^{25b} are taken together with their intervening atom to form an optionally substituted, monocyclic, carbocyclic or heterocyclic ring. In some embodiments, R^{25a} and R^{25b} are taken together with their intervening atom to form an optionally substituted, monocyclic, carbocyclic ring. In some embodiments, R^{25a} and R^{25b} are taken together with their intervening atom to form an optionally substituted, monocyclic, C₃₋₁₄ carbocyclic ring. In some embodiments, R^{25a} and R^{25b} are taken together with their intervening atom to form an optionally substituted, monocyclic, C₃₋₇ carbocyclic ring. In some embodiments, R^{25a} and R^{25b} are taken together with their intervening atom to form an optionally substituted, monocyclic, heterocyclic ring. In some embodiments, R^{25a} and R^{25b} are taken together with their intervening atom to form an optionally substituted, monocyclic, 3- to 14-membered heterocyclic ring. In some embodiments, R^{25a} and R^{25b} are taken together with their intervening atom to form an optionally substituted, monocyclic, 3- to 7-membered heterocyclic ring.

In some embodiments, R^{25b} and R^{26a} are taken together with their intervening atom to form an optionally substituted, monocyclic, carbocyclic or heterocyclic ring. In some embodiments, R^{25b} and R^{26a} are taken together with their intervening atom to form an optionally substituted, monocyclic, C₃₋₁₄ carbocyclic ring. In some embodiments, R^{25b} and R^{26a} are taken together with their intervening atom to form an optionally substituted, monocyclic, C₃₋₇ carbocyclic ring. In some embodiments, R^{25b} and R^{26a} are taken together with their intervening atom to form an optionally substituted, monocyclic, 3- to 14-membered heterocyclic ring. In some embodiments, R^{25b} and R^{26a} are taken together with their intervening atom to form an optionally substituted, monocyclic, 3- to 7-membered heterocyclic ring.

In some embodiments, R^{26a} and R^{26b} are taken together with their intervening atom to form an optionally substituted, monocyclic, carbocyclic or heterocyclic ring. In some embodiments, R^{26a} and R^{26b} are taken together with their intervening atom to form an optionally substituted, monocyclic, C₃₋₁₄ carbocyclic ring. In some embodiments, R^{26a} and R^{26b} are taken together with their intervening atom to form an optionally substituted, monocyclic, C₃₋₇ carbocyclic ring. In some embodiments, R^{26a} and R^{26b} are taken together with their intervening atom to form an optionally substituted, monocyclic, 3- to 14-membered heterocyclic ring. In some embodiments, R^{26a} and R^{26b} are taken together with their intervening atom to form an optionally substituted, monocyclic, 3- to 7-membered heterocyclic ring.

In some embodiments, Formula III-A or III-B is any one of the formulae shown in Table 5:

TABLE 5

| | |
|---|---|
| 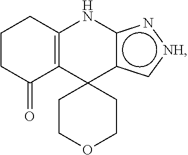 | 884111 |
| 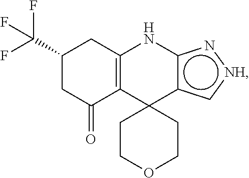 | 901477 |
| 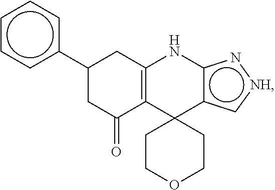 | 893309 |
| 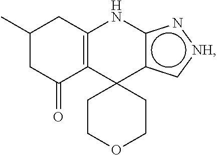 | 884112 |
| 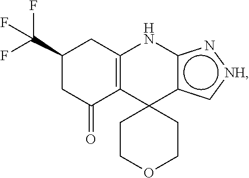 | 901478 |
| 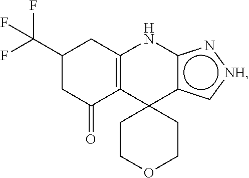 | 889572 |
| 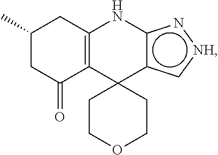 | 912078 |
| 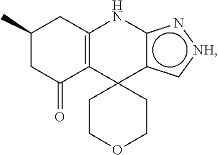 | 912079 |
| 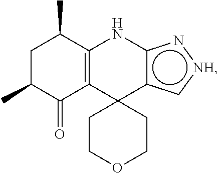 | 893316 |
| 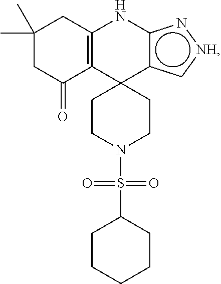 | 913128 |
| 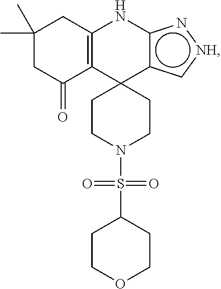 | 913127 |
| 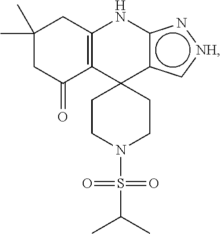 | 913129 |
| 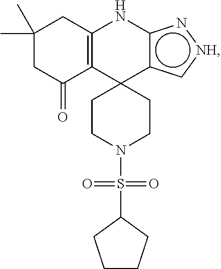 | 909206 |
| 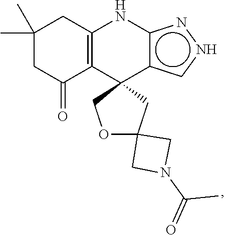 | 926861 |
| 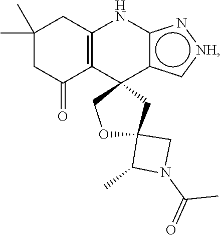 | 948437 |
| 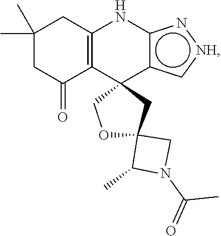 | 948439 |
| 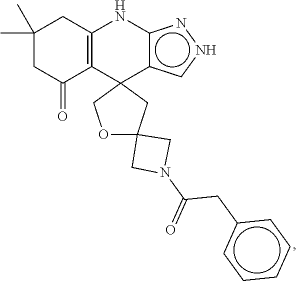 | 947306 |
| 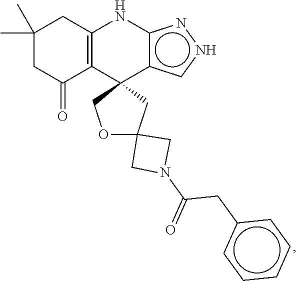 | 948037 |
| 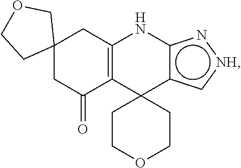 | 889573 |
| 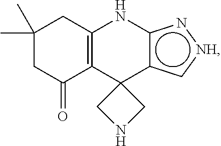 | 912075 |
| 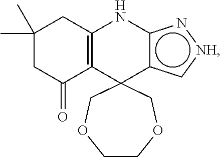 | 912094 |
| 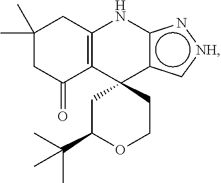 | 912113 |
| 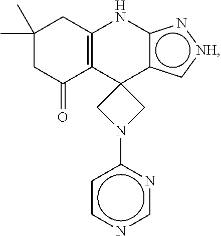 | 909209 |
| 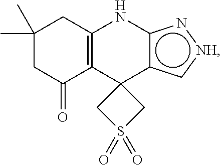 | 912170 |
| 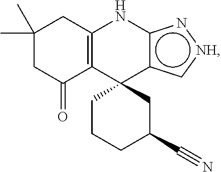 | 946078 |
| 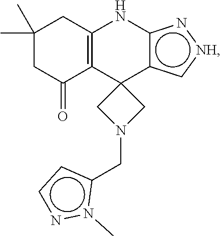 | 932653 |
| 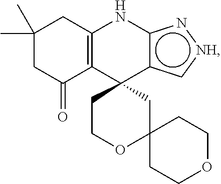 | 942301 |
| 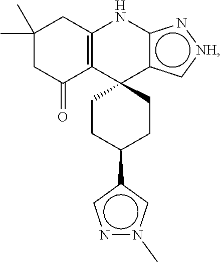 | 949323 |
| 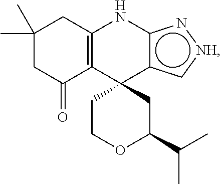 | 884013 |
| 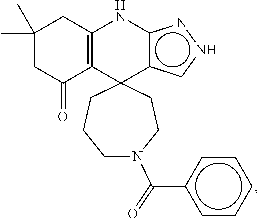 | 889577 |
| 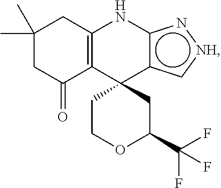 | 884023 |
| 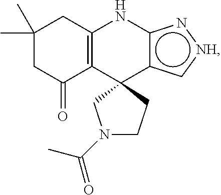 | 889583 |
| 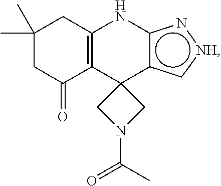 | 893314 |
| 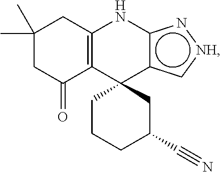 | 946080 |
| 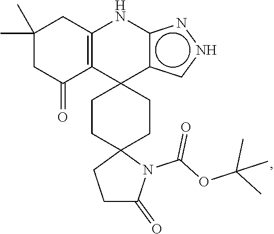 | 912217 |
| 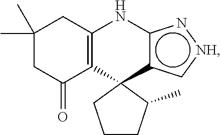 | 874319 |
| 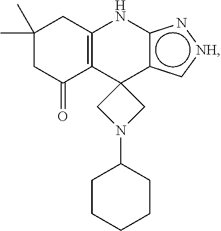 | 929446 |
| 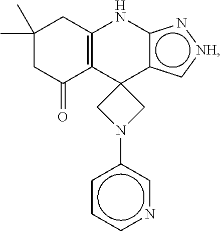 | 947813 |
| 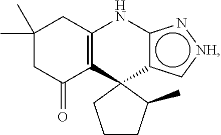 | 874320 |
| 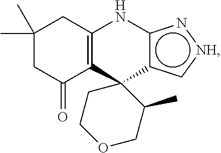 | 924309 |
| 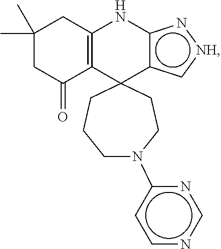 | 917145 |
| 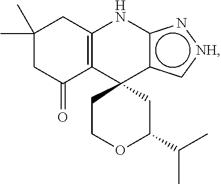 | 884014 |
| 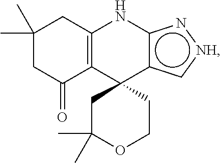 | 924298 |
| 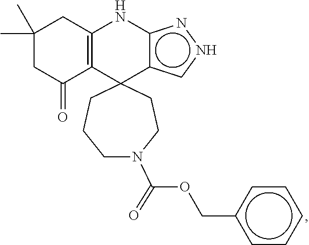 | 884016 |
| 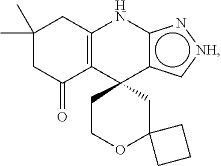 | 897755 |
| 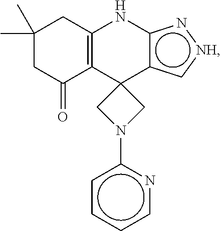 | 945477 |
| 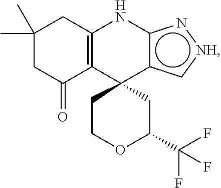 | 884024 |
| 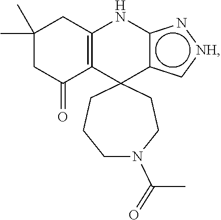 | 889575 |
| 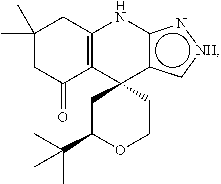 | 912106 |
| 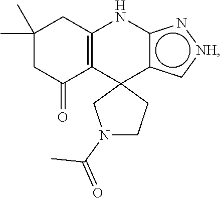 | 826763 |
| 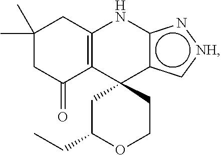 | 907328 |
| 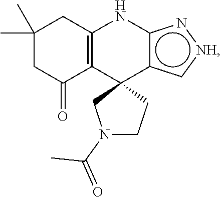 | 889584 |
| 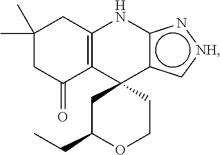 | 907329 |
| 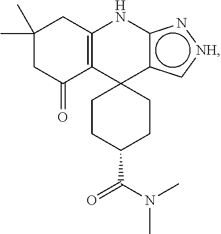 | 945395 |
| 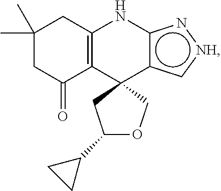 | 913184 |
| 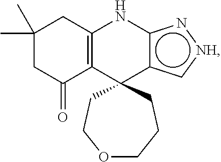 | 874536 |
| 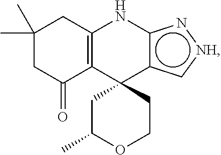 | 899559 |
| 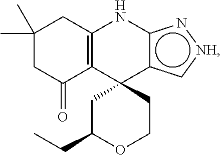 | 924311 |
| 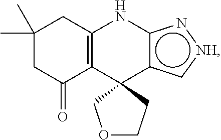 | 877314 |
| 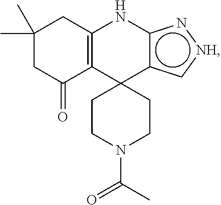 | 826649 |
| 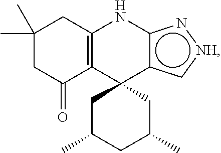 | 884113 |
| 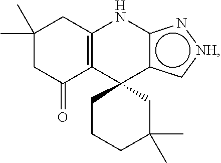 | 883846 |
| 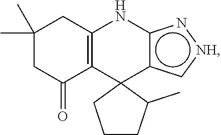 | 826765 |
| 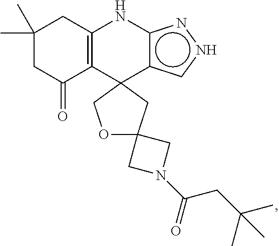 | 947301 |
| 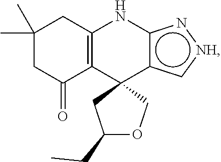 | 913214 |
| 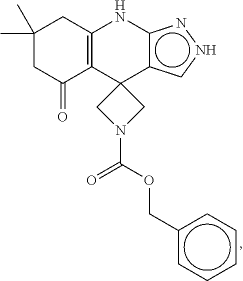 | 889568 |
| 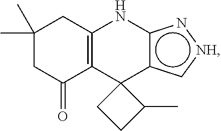 | 949640 |
| 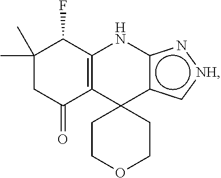 | 883833 |
| 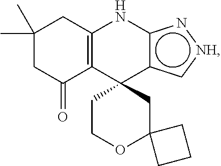 | 897756 |
| 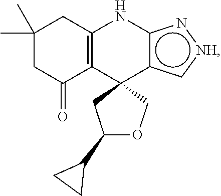 | 913185 |
| 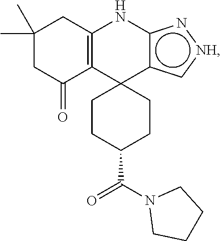 | 945394 |
| 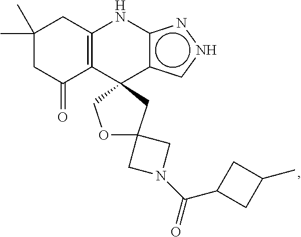 | 947732 |
| 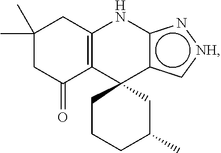 | 874537 |
| 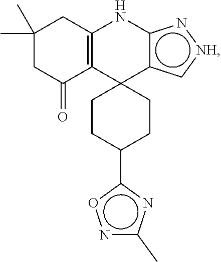 | 943895 |
| 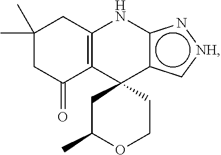 | 899560 |
| 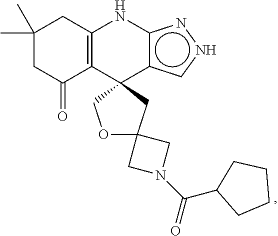 | 943958 |
| 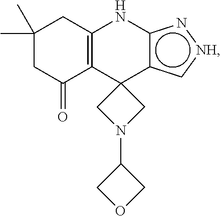 | 912076 |
| 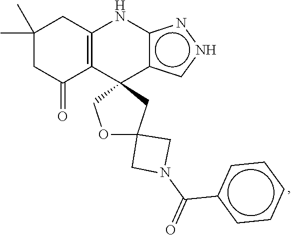 | 943956 |
| 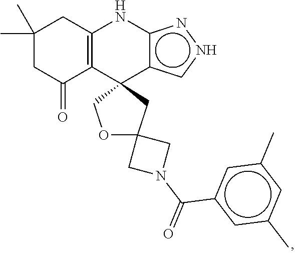 | 948222 |
| 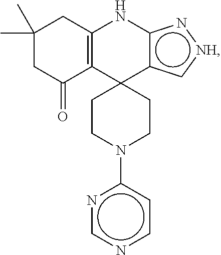 | 874529 |
| 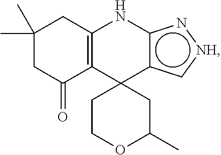 | 883794 |
| 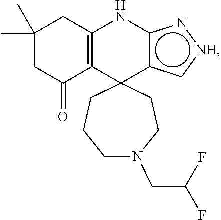 | 889574 |
| 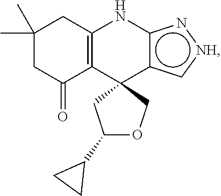 | 913185 |
| 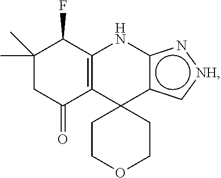 | 883834 |
| 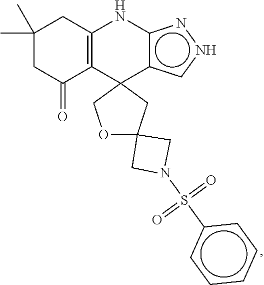 | 913002 |
| 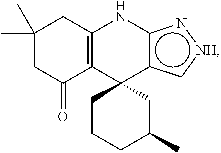 | 893319 |
| 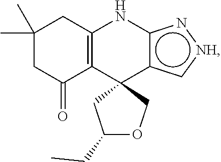 | 913215 |
| 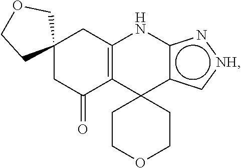 | 901475 |
| 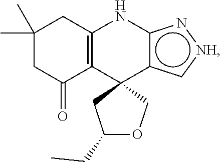 | 913213 |
| 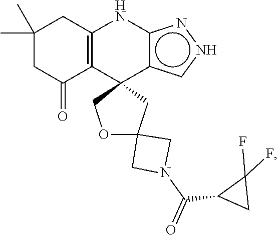 | 948217 |
| 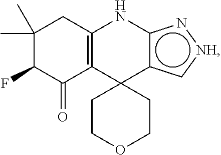 | 883836 |
| 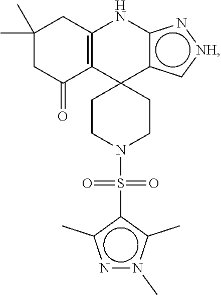 | 947257 |
| 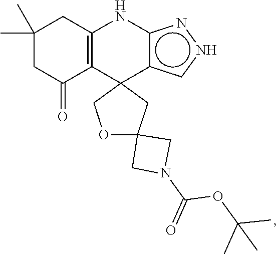 | 911083 |
| 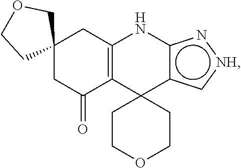 | 901476 |
| 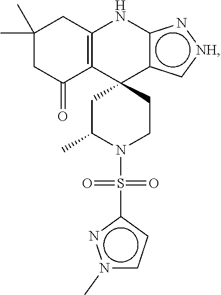 | 917160 |
| 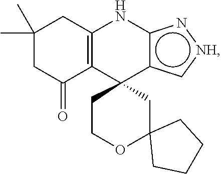 | 907326 |
| 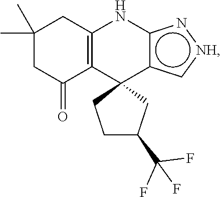 | 952434 |
| 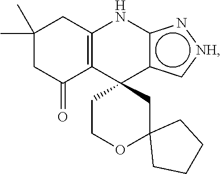 | 907327 |
| 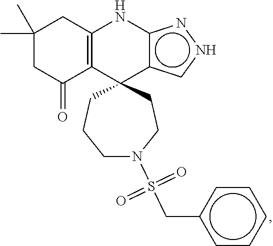 | 945483 |
| 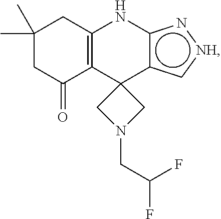 | 912073 |
| 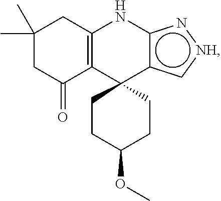 | 884120 |
| 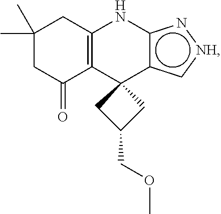 | 912173 |
| 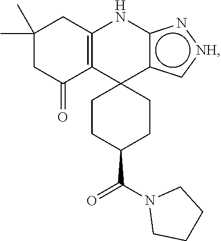 | 945396 |
| 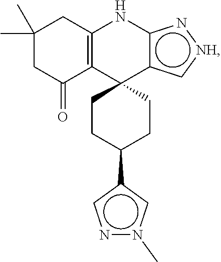 | 949322 |
| 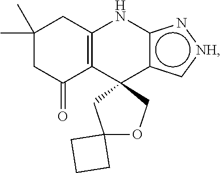 | 917129 |
| 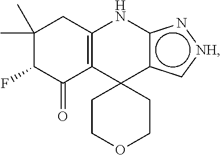 | 993935 |
| 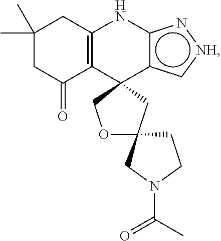 | 948320 |
| 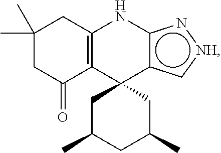 | 884114 |
| 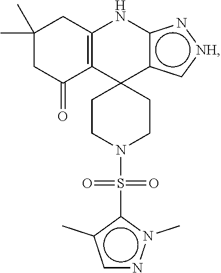 | 945658 |
| 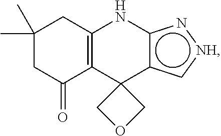 | 889569 |
| 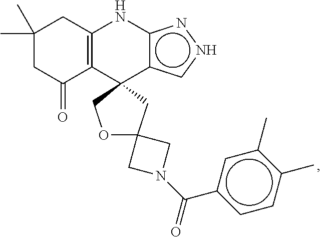 | 948039 |
| 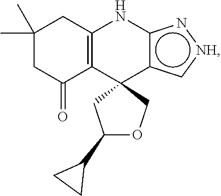 | 913442 |
| 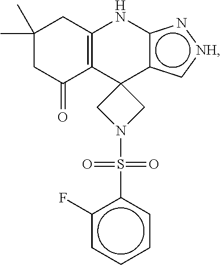 | 913263 |
| 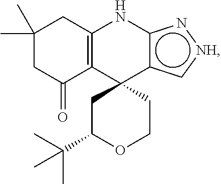 | 912112 |
| 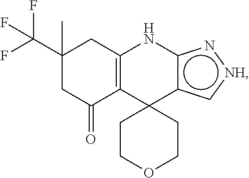 | 901458 |
| 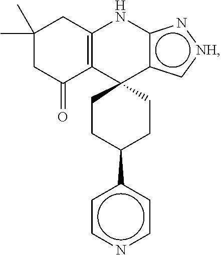 | 949320 |
| 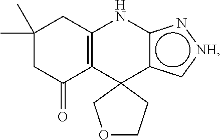 | 826713 |
| 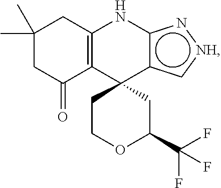 | 884022 |
| 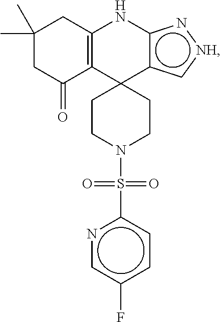 | 947251 |
| 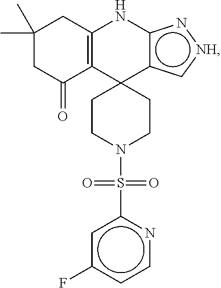 | 947262 |
| 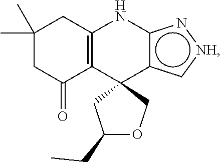 | 913216 |
| 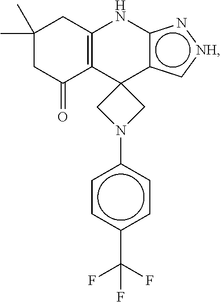 | 945478 |
| 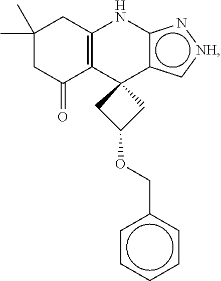 | 909210 |
| 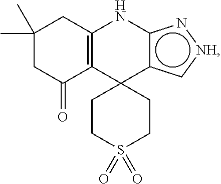 | 896013 |
| 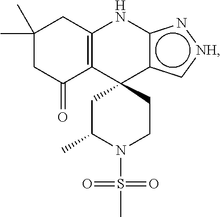 | 918315 |
| 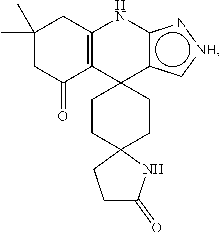 | 913056 |
| 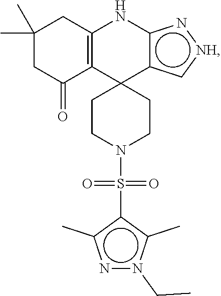 | 945679 |
| 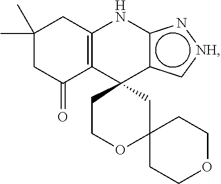 | 924306 |
| 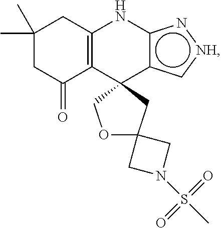 | 926823 |
| 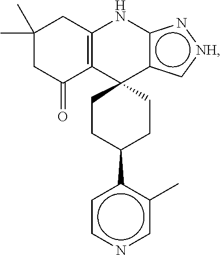 | 949318 |
| 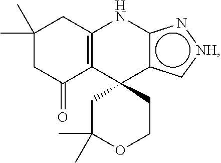 | 924299 |
| 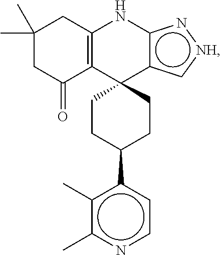 | 950477 |
| 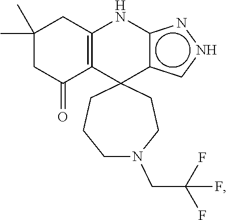 | 889576 |
| 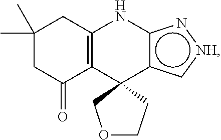 | 877313 |
| 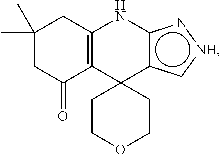 | 826648 |
| 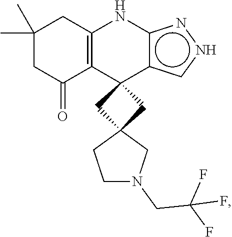 | 913138 |
| 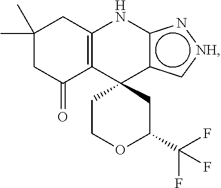 | 884021 |
| 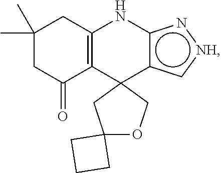 | 911973 |
| 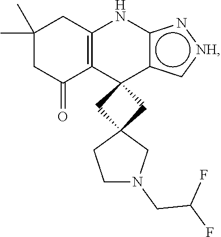 | 913139 |
| 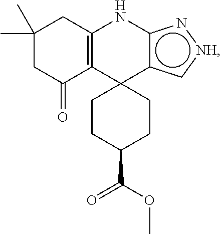 | 918270 |
| 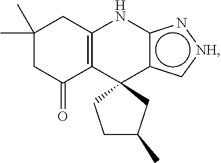 | 883840 |
| 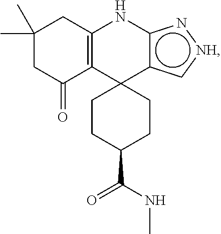 | 945397 |
| 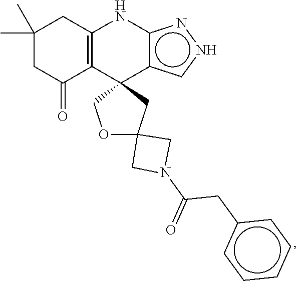 | 948036 |
| 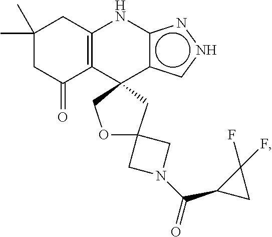 | 948221 |
| 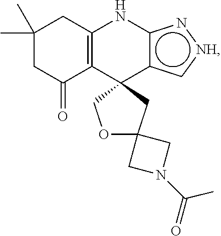 | 926872 |
| 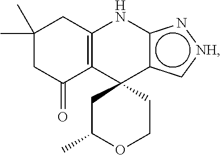 | 924308 |
| 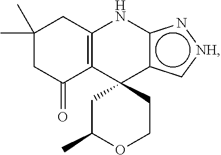 | 924307 |
| 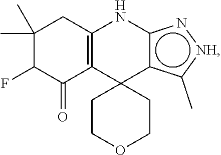 | 912072 |
| 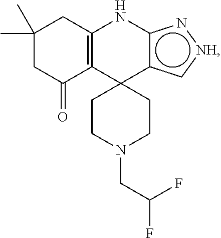 | 883962 |
| 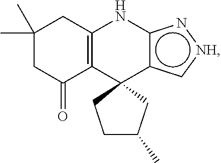 | 883841 |
| 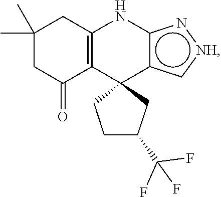 | 952437 |
| 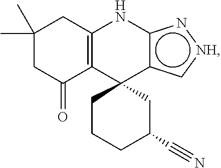 | 945906 |
| 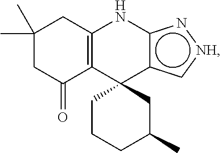 | 893318 |
| 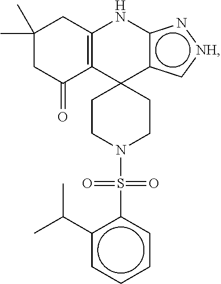 | 947473 |
| 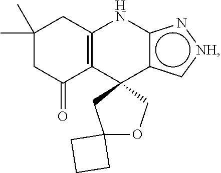 | 917128 |
| 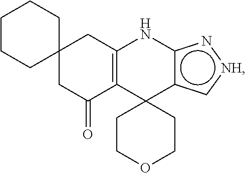 | 893307 |
| 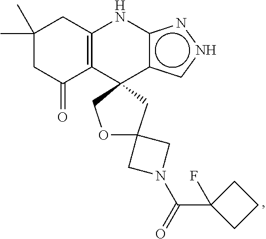 | 947736 |
| 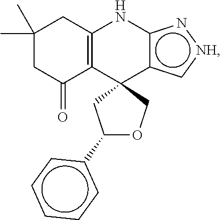 | 946072 |
| 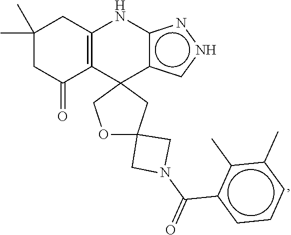 | 947284 |
| 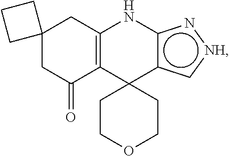 | 909208 |
| 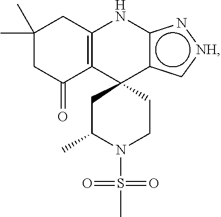 | 918316 |
| 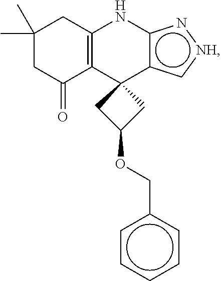 | 909211 |
| 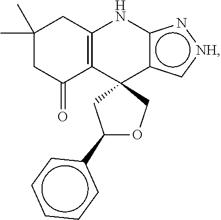 | 946073 |
| 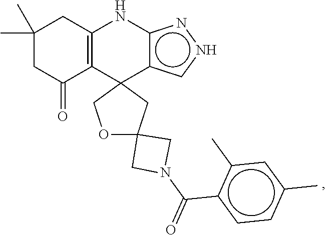 | 947294 |
| 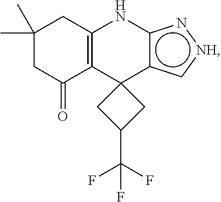 | 874527 |
| 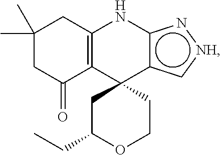 | 924715 |
| 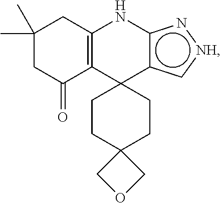 | 883977 |
| 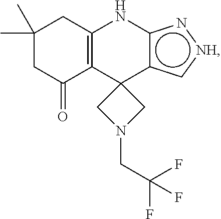 | 912074 |
| 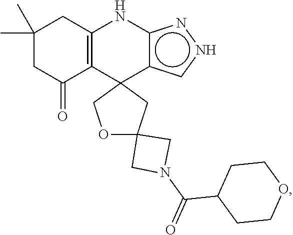 | 945407 |
| 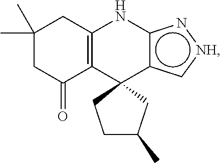 | 897751 |
| 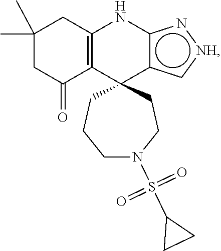 | 932659 |
| 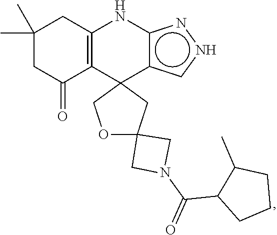 | 947274 |
| 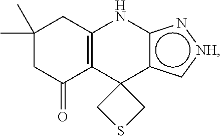 | 889567 |
| 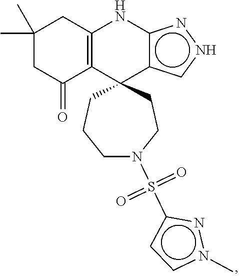 | 932660 |
| 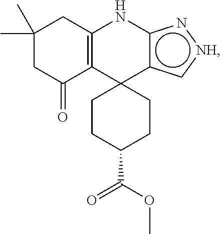 | 918277 |
| 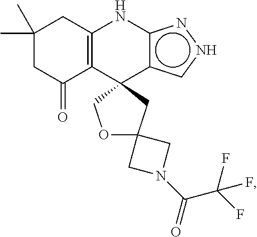 | 948156 |
| 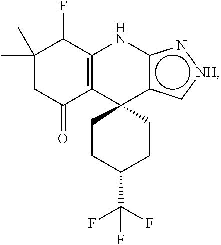 | 917136 |
| 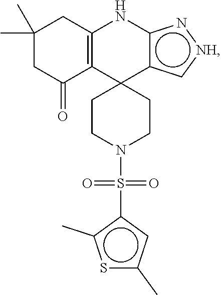 | 945674 |
| 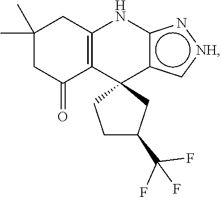 | 952435 |
| 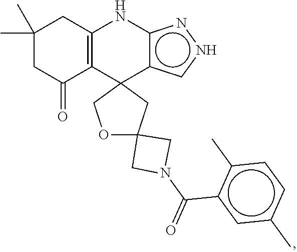 | 947285 |
| 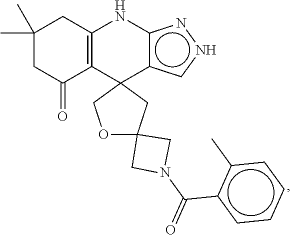 | 947287 |
| 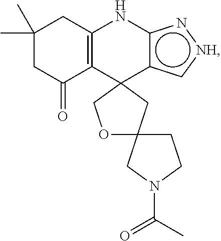 | 948040 |
| 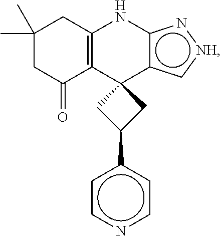 | 889664 |
| 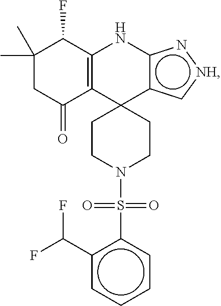 | 951839 |
| 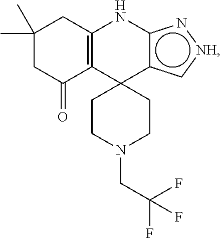 | 874528 |
| 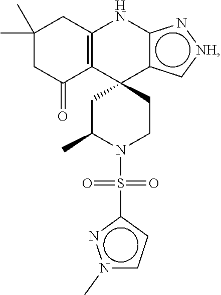 | 926832 |
| 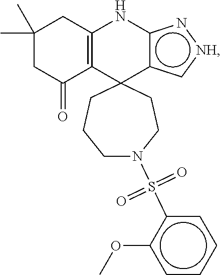 | 917142 |
| 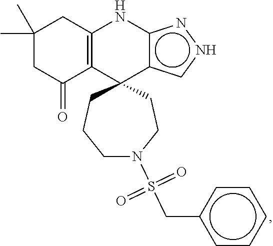 | 945473 |
| 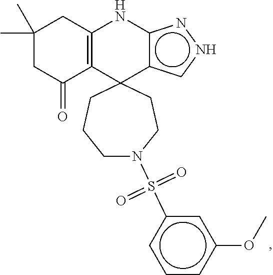 | 917140 |
| 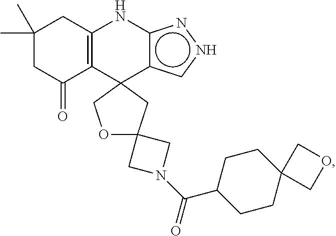 | 944075 |
| 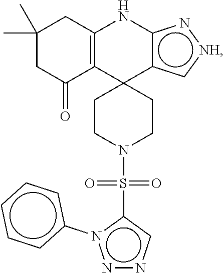 | 947466 |
| 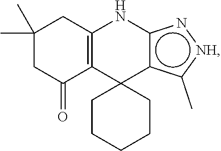 | 883865 |
| 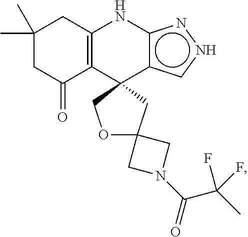 | 947839 |
| 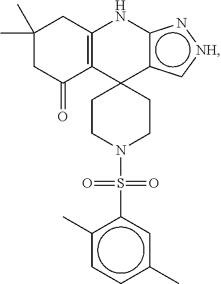 | 947471 |
| 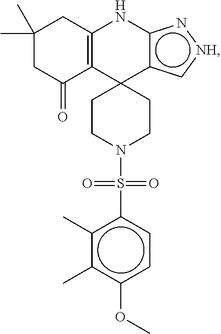 | 945662 |
| 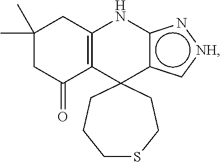 | 913126 |
| 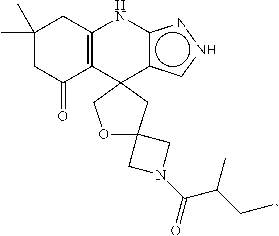 | 947270 |
| 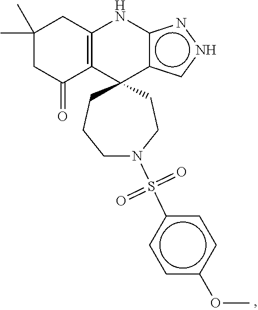 | 932641 |
| 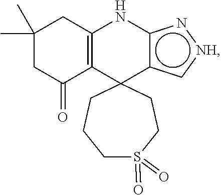 | 927343 |
| 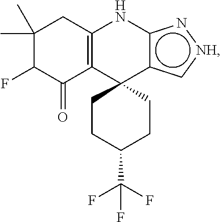 | 917134 |
| 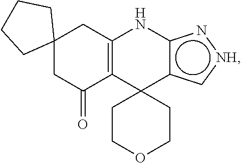 | 893308 |
| 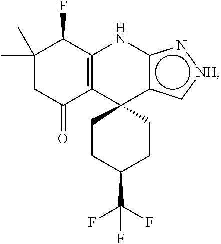 | 918284 |
| 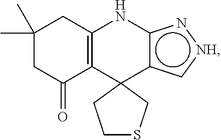 | 883973 |
| 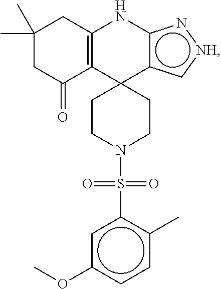 | 947245 |
| 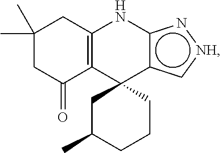 | 874532 |
| 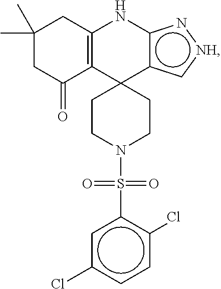 | 948122 |
| 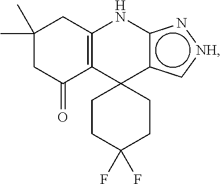 | 883972 |
| 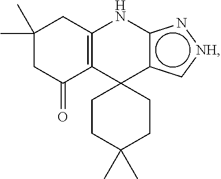 | 884107 |
| 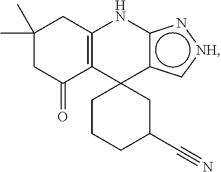 | 923675 |
| 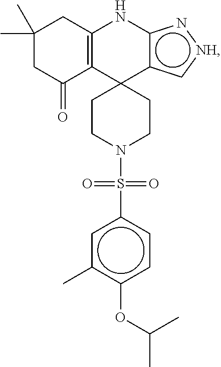 | 945678 |
| 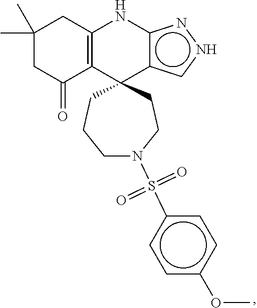 | 932661 |
| 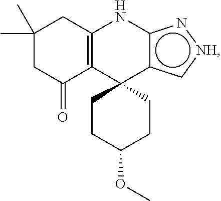 | 884119 |
| 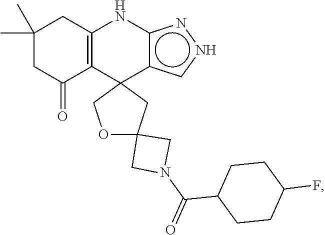 | 947267 |
| 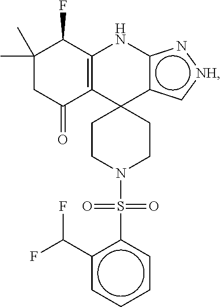 | 951846 |
| 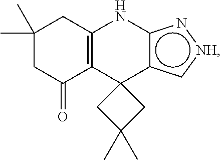 | 883864 |
| 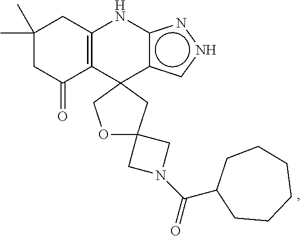 | 947308 |
| 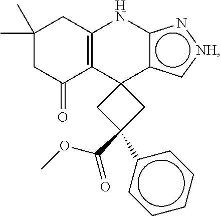 | 942344 |
| 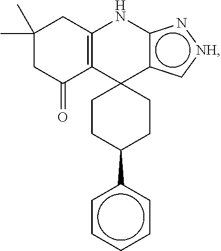 | 874975 |
| 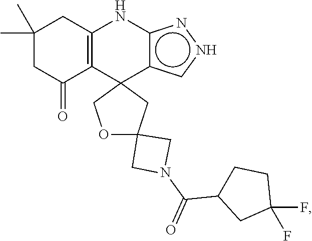 | 947265 |
| 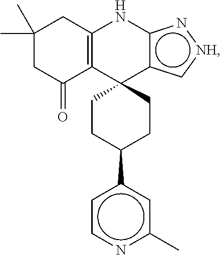 | 948416 |
| 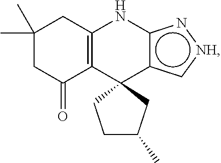 | 897750 |
| 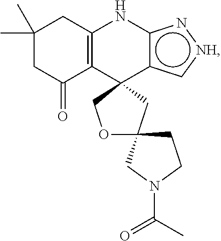 | 948319 |
| 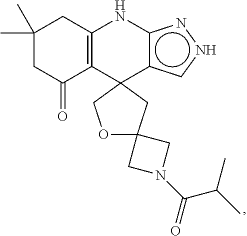 | 947304 |
| 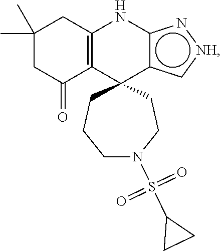 | 932639 |
| 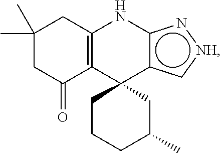 | 893320 |
| 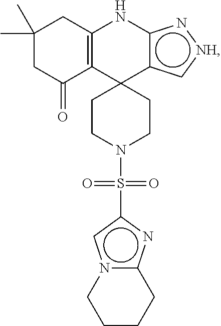 | 947472 |
| 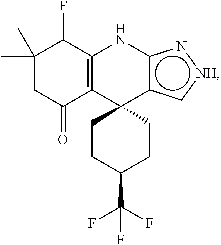 | 917135 |
| 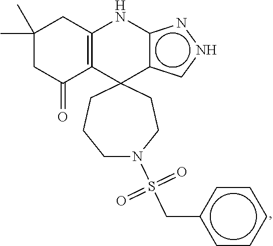 | 932632 |
| 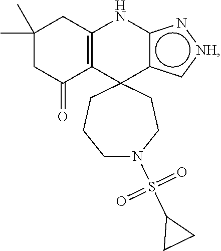 | 917137 |
| 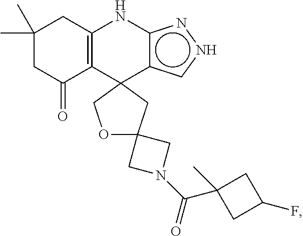 | 947271 |
| 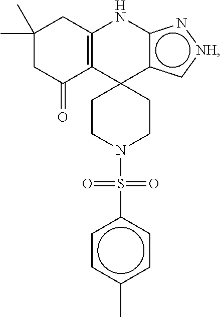 | 947459 |
| 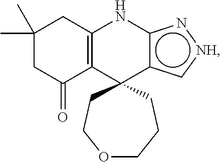 | 874535 |
| 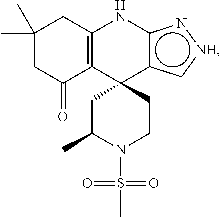 | 926827 |
| 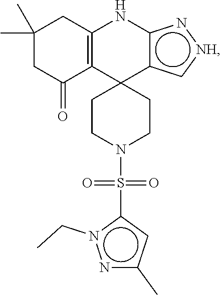 | 947609 |
| 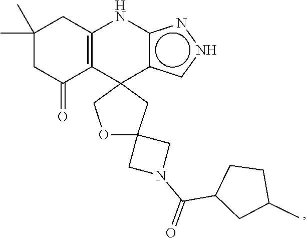 | 947268 |
| 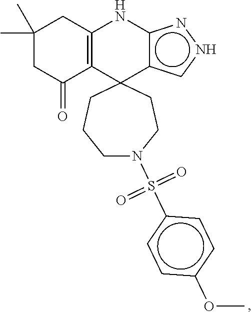 | 917141 |
| 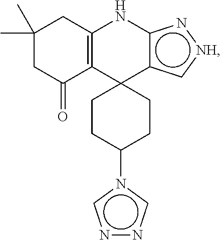 | 943894 |
| 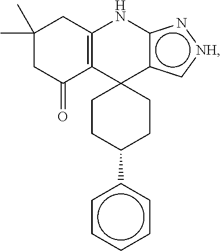 | 912084 |
| 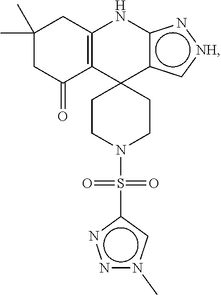 | 947898 |
| 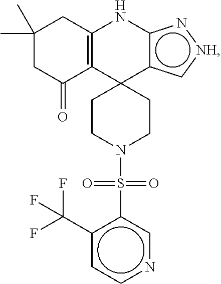 | 947463 |
| 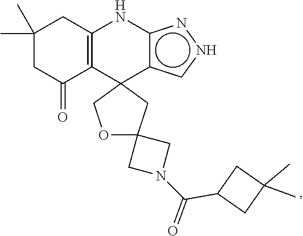 | 947282 |
| 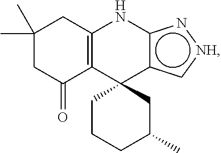 | 893321 |
| 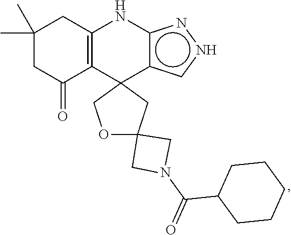 | 947291 |
| 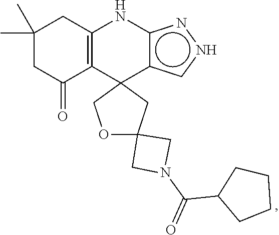 | 917232 |
| 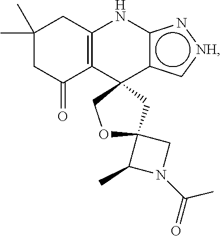 | 948438 |
| 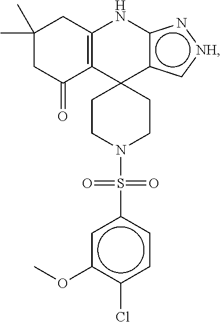 | 945669 |
| 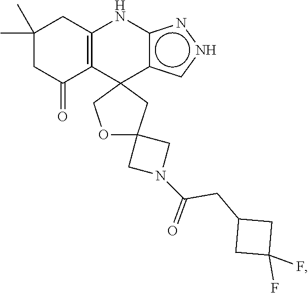 | 947278 |
| 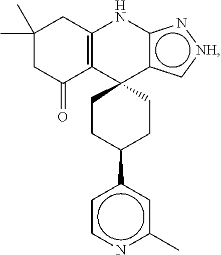 | 948415 |
| 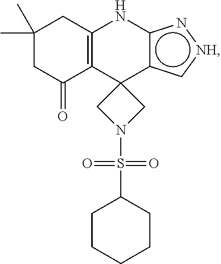 | 918275 |
| 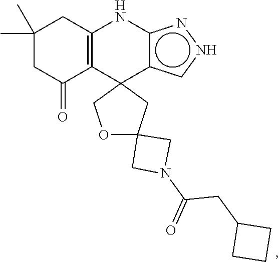 | 947299 |
| 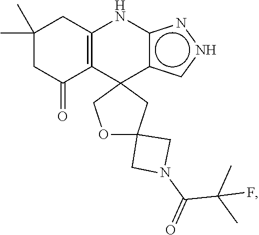 | 947276 |
| 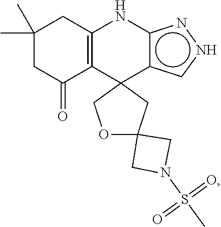 | 913057 |
| 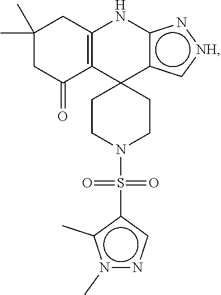 | 947460 |
| 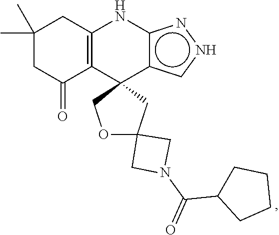 | 943957 |
| 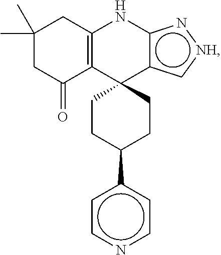 | 949321 |
| 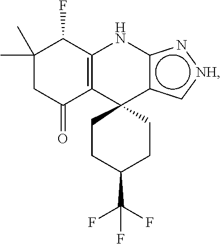 | 918285 |
| 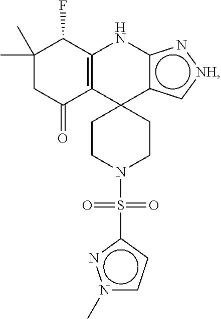 | 951708 |
| 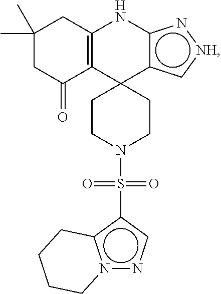 | 947474 |
| 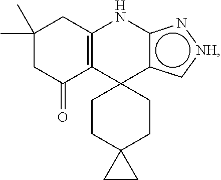 | 893312 |
| 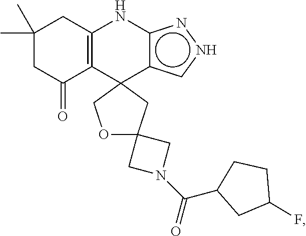 | 947486 |
| 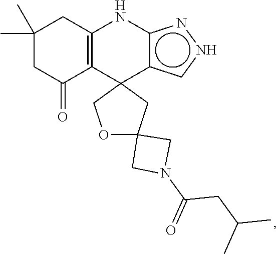 | 947302 |
| 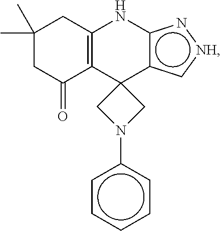 | 948298 |
| 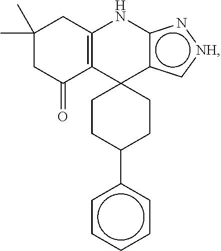 | 877320 |
| 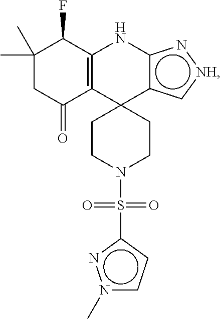 | 951709 |
| 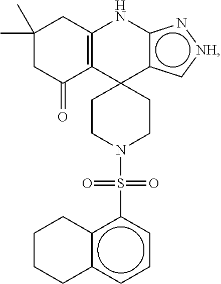 | 947470 |
| 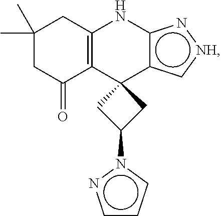 | 884005 |
| 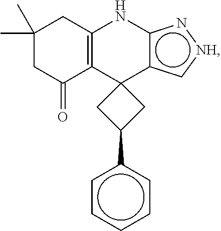 | 917081 |
| 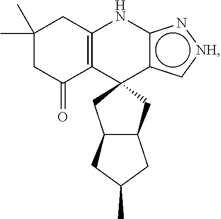 | 943898 |
| 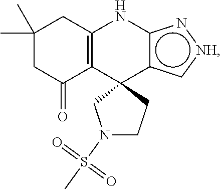 | 942462 |
| 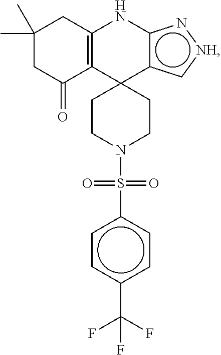 | 947256 |
| 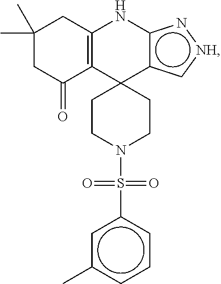 | 947261 |
| 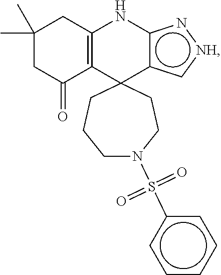 | 889578 |
| 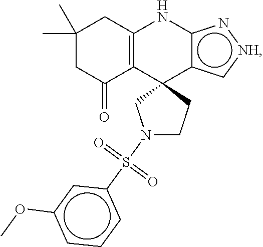 | 951790 |
| 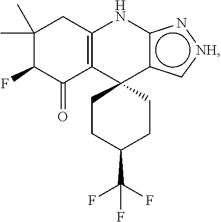 | 918282 |
| 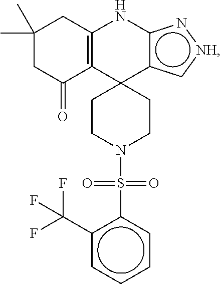 | 945667 |
| 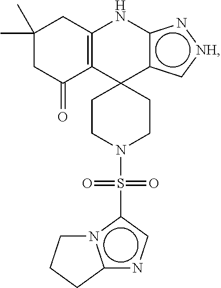 | 947467 |
| 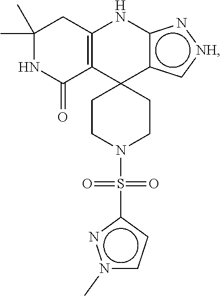 | 943961 |
| 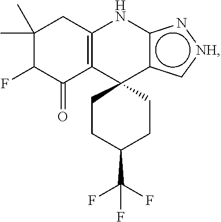 | 917133 |
| 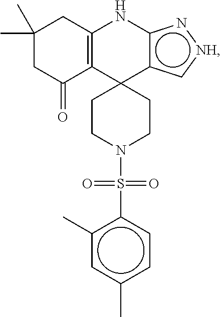 | 947468 |
| 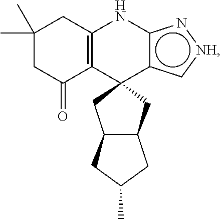 | 943896 |
| 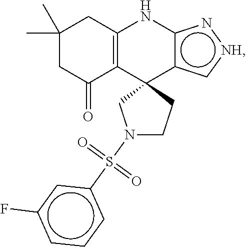 | 951862 |
| 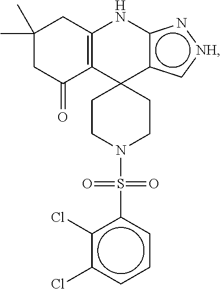 | 947249 |
| 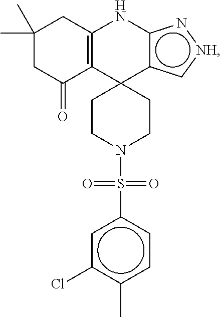 | 947241 |
| 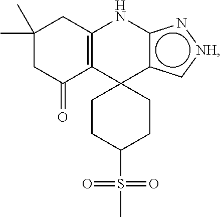 | 913356 |
| 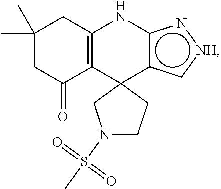 | 912092 |
| 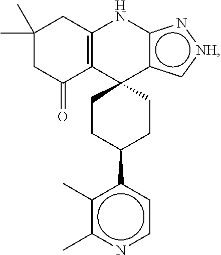 | 950478 |
| 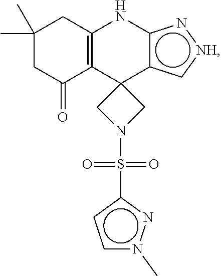 | 913260 |
| 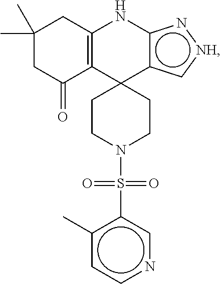 | 945675 |
| 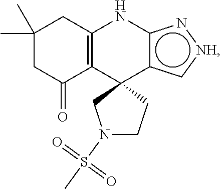 | 942432 |
| 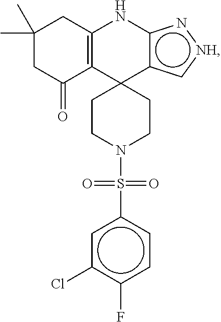 | 948117 |
| 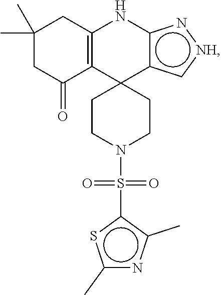 | 947259 |
| 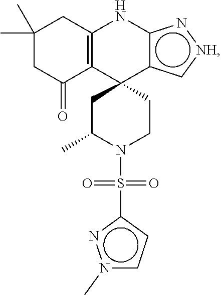 | 917161 |
| 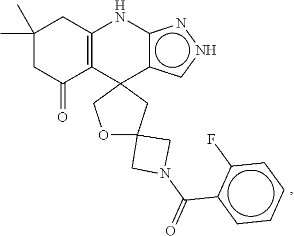 | 947289 |
| 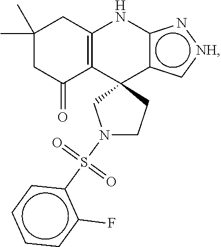 | 945719 |
| 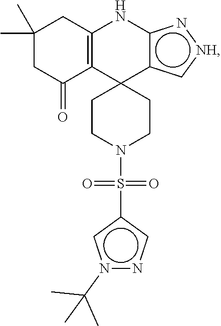 | 945677 |
| 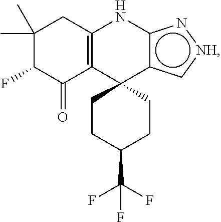 | 918283 |
| 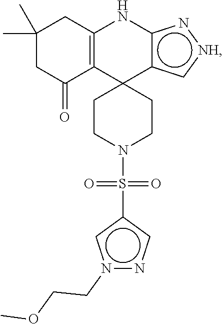 | 947477 |
| 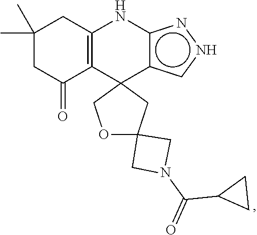 | 947297 |
| 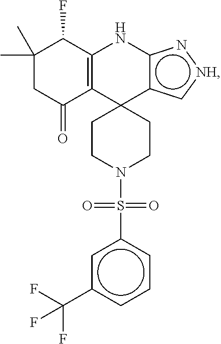 | 951713 |
| 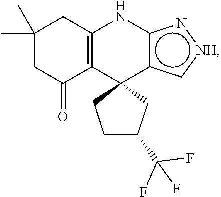 | 952436 |
| 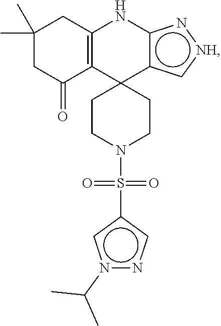 | 947607 |
| 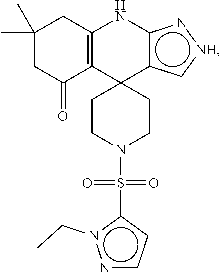 | 945664 |
| 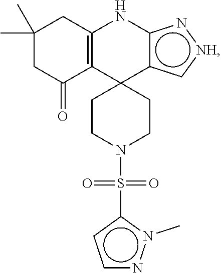 | 932649 |
| 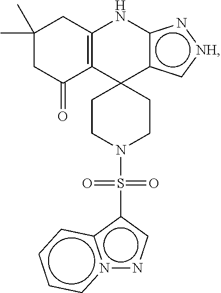 | 947615 |
| 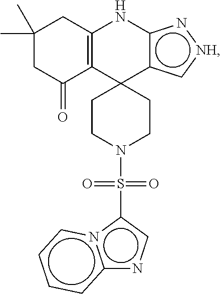 | 947610 |
| 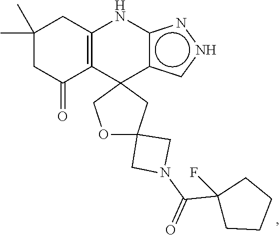 | 947277 |
| 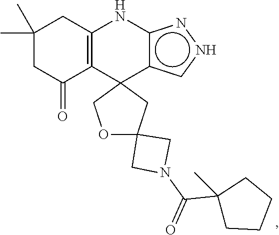 | 947307 |
| 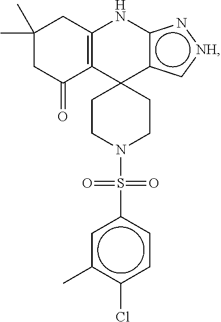 | 947475 |
| 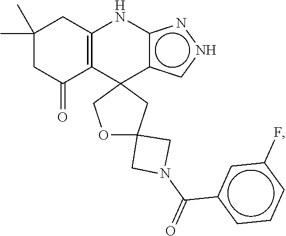 | 947283 |
| 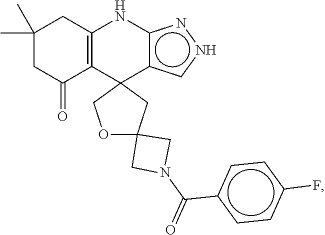 | 947292 |
| 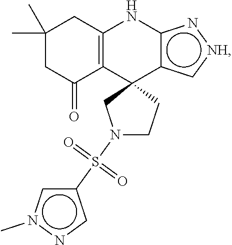 | 943483 |
| 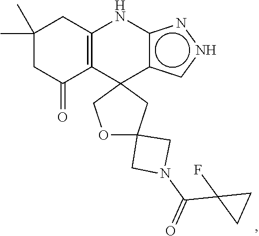 | 947487 |
| 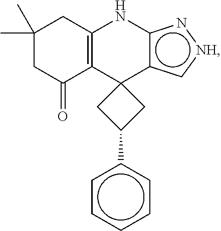 | 917082 |
| 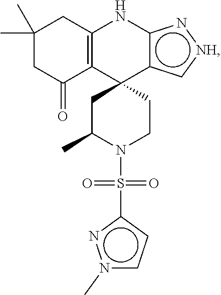 | 926815 |
| 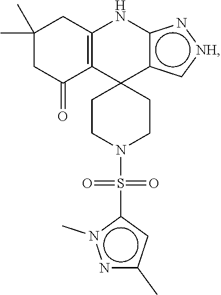 | 947608 |
| 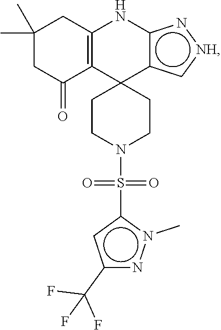 | 947606 |
| 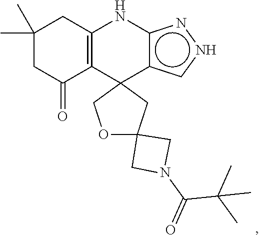 | 947303 |
| 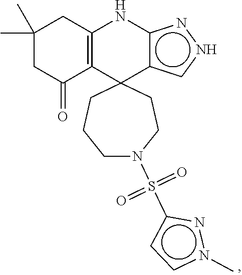 | 917138 |
| 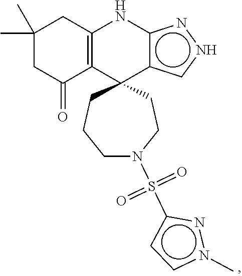 | 932640 |
| 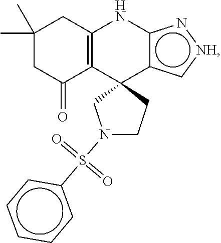 | 951788 |
| 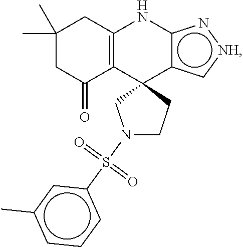 | 951864 |
| 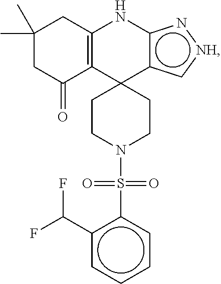 | 945657 |
| 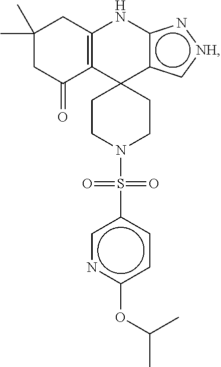 | 945676 |
| 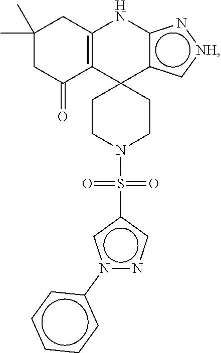 | 947904 |
| 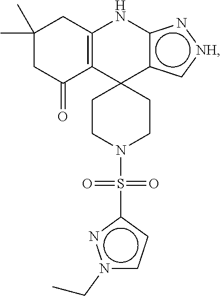 | 945671 |
| 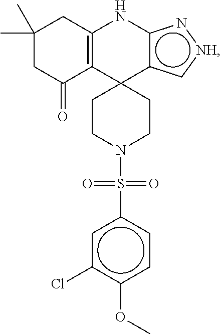 | 947612 |
| 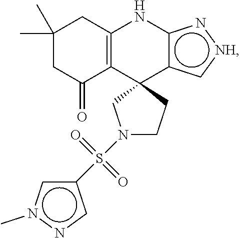 | 951726 |
| 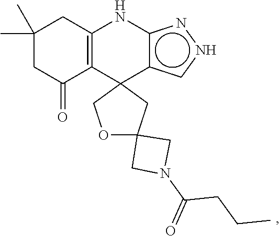 | 947300 |
| 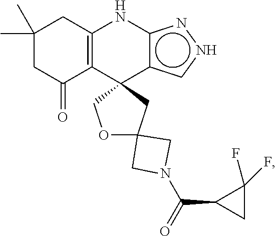 | 948219 |
| 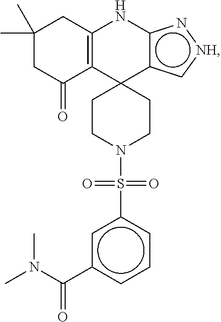 | 947247 |
| 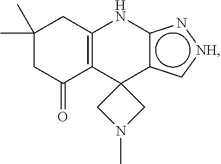 | 913264 |
| 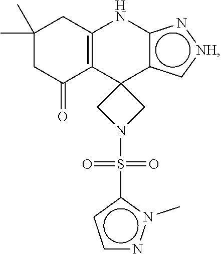 | 932648 |
| 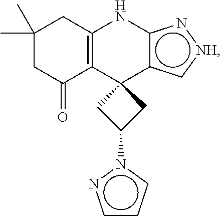 | 884004 |
| 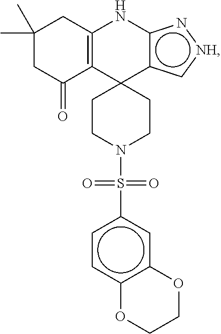 | 947900 |
| 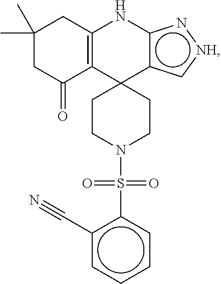 | 947479 |
| 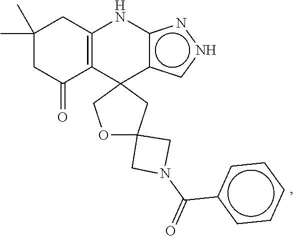 | 917231 |
| 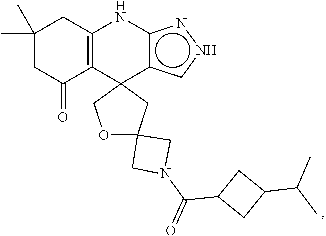 | 947273 |
| 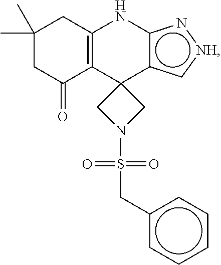 | 943543 |
| 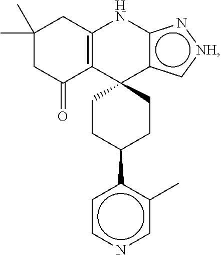 | 949319 |
| 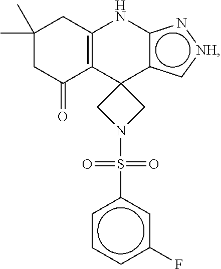 | 918278 |
| 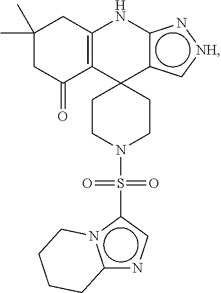 | 947480 |
| 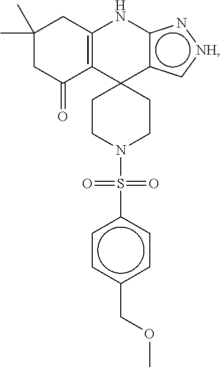 | 947250 |
| 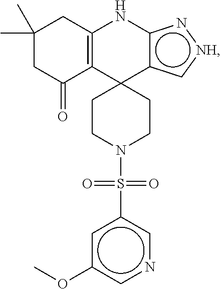 | 947465 |
| 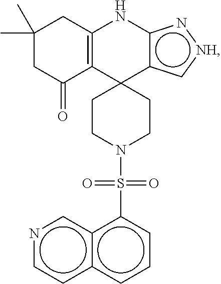 | 947901 |
| 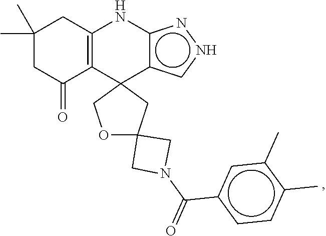 | 947286 |
| 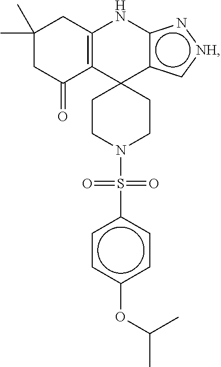 | 945673 |
| 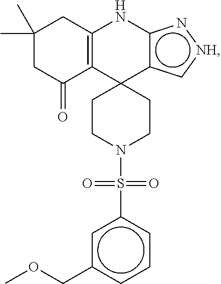 | 947605 |
| 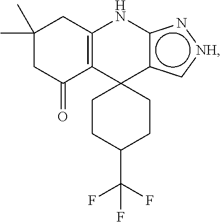 | 865193 |
| 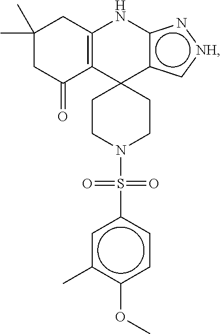 | 945680 |
| 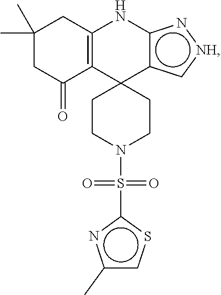 | 947897 |
| 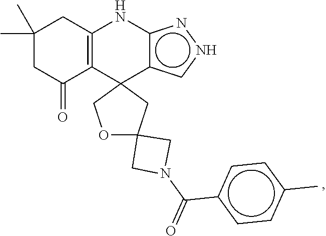 | 947290 |
| 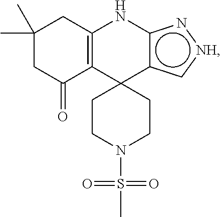 | 880776 |
| 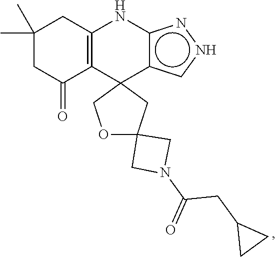 | 947298 |
| 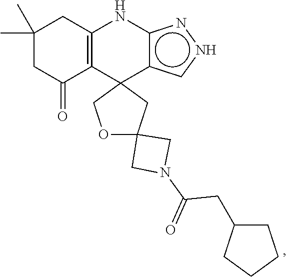 | 947293 |
| 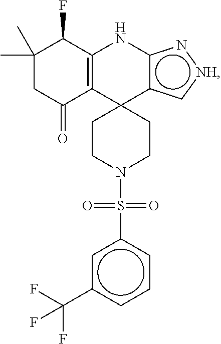 | 951703 |
| 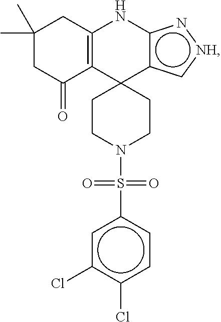 | 945660 |
| 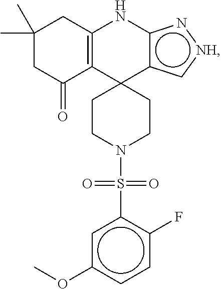 | 945665 |
| 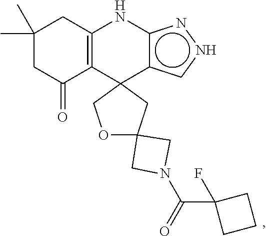 | 947279 |
| 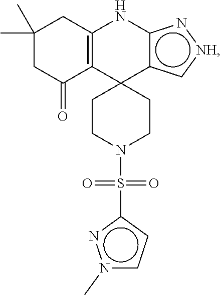 | 901471 |
| 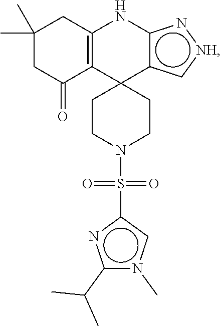 | 945663 |
| 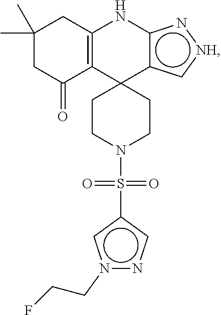 | 947903 |
| 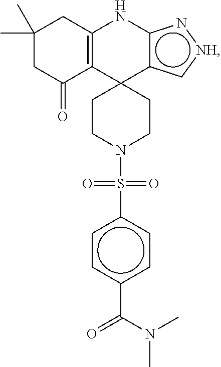 | 945659 |
| 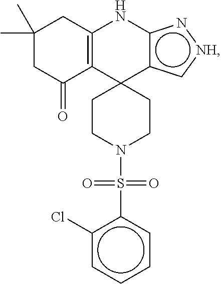 | 945655 |
| 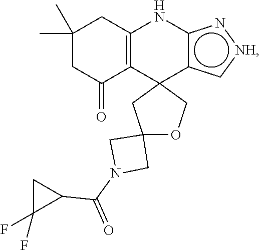 | 947272 |
| 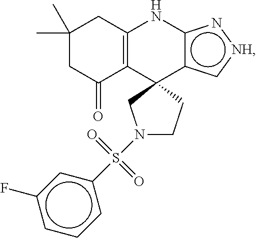 | 943488 |
| 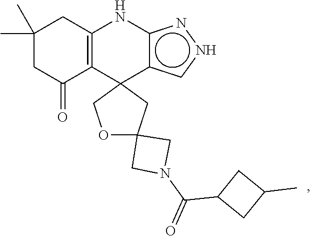 | 947266 |
| 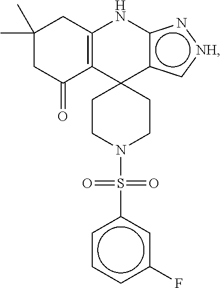 | 901466 |
| 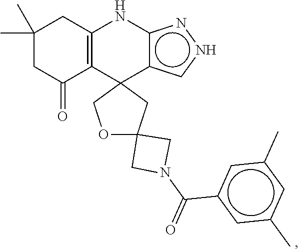 | 947296 |
| 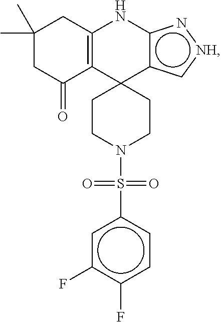 | 948119 |
| 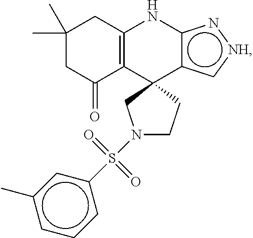 | 943489 |
| 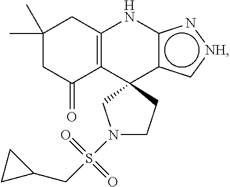 | 951724 |
| 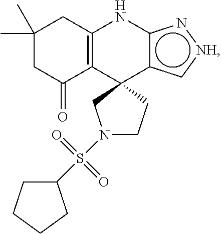 | 943482 |
| 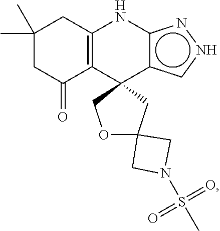 | 926808 |
| 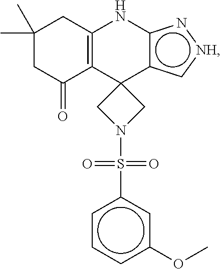 | 918276 |
| 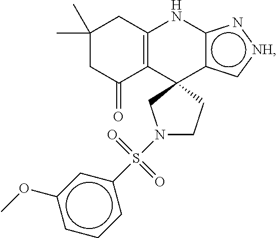 | 943486 |
| 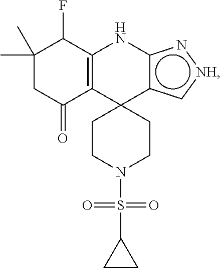 | 950814 |
| 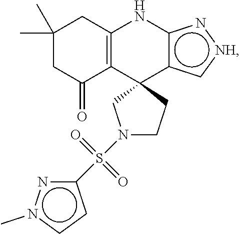 | 945714 |
| 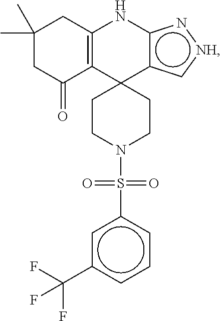 | 947244 |
| 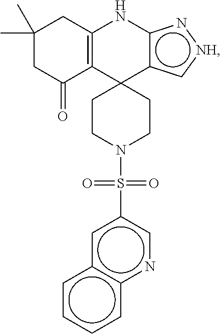 | 947462 |
| 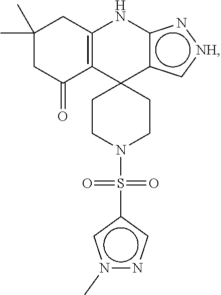 | 901467 |
| 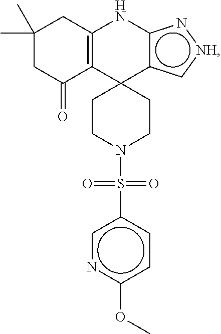 | 945666 |
| 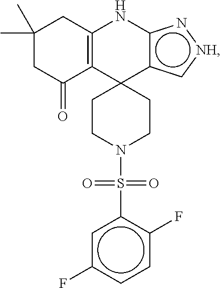 | 948121 |
| 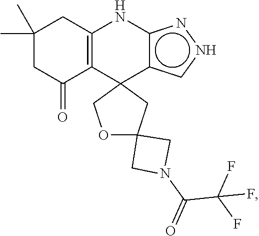 | 947280 |
| 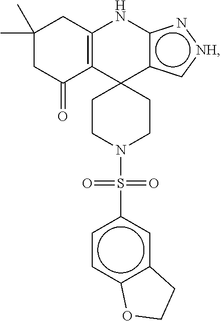 | 913251 |
| 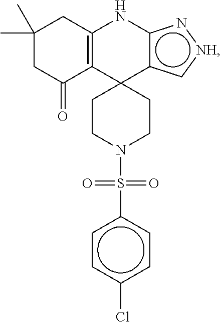 | 945661 |
| 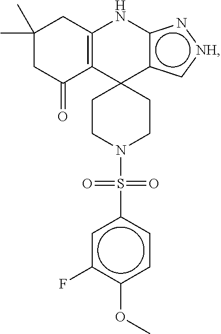 | 947478 |
| 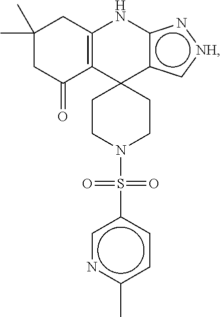 | 947482 |
| 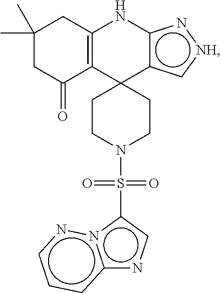 | 948120 |
| 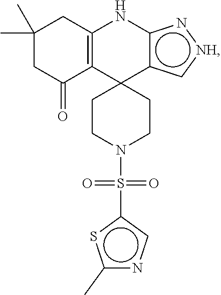 | 947899 |
| 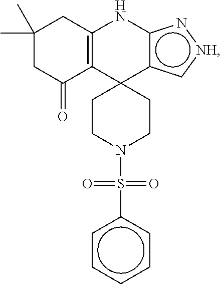 | 874530 |
| 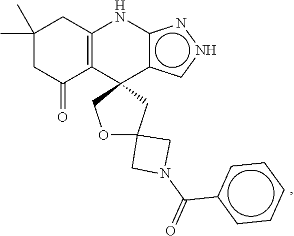 | 943955 |
| 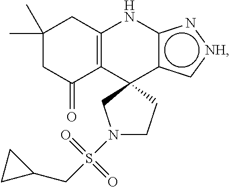 | 943481 |
| 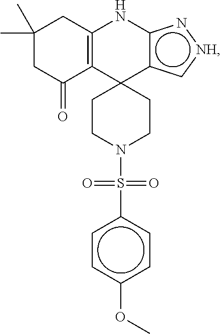 | 917143 |
| 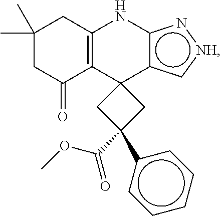 | 942366 |
| 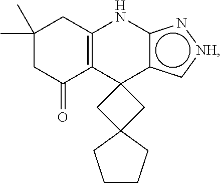 | 874477 |
| 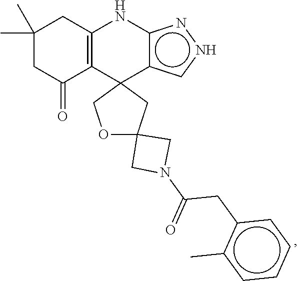 | 947295 |
| 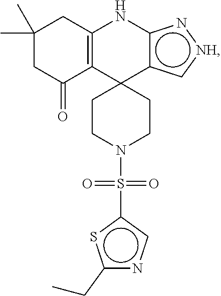 | 947252 |
| 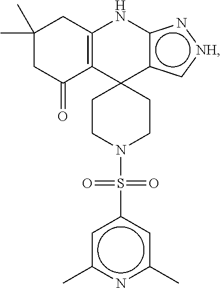 | 947616 |
| 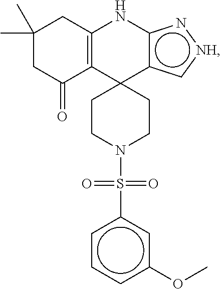 | 901468 |
| 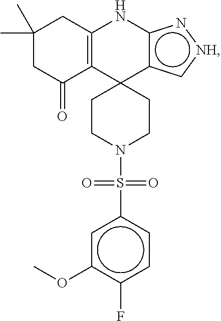 | 947263 |
| 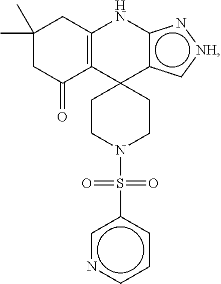 | 947613 |
| 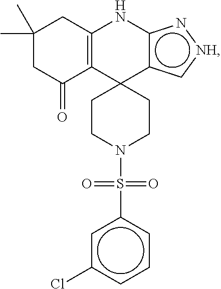 | 947243 |
| 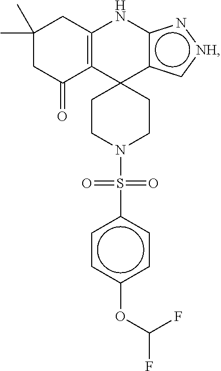 | 947246 |
| 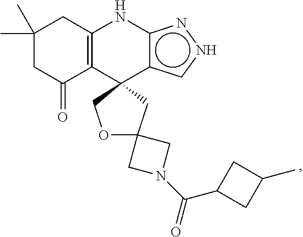 | 947733 |
| 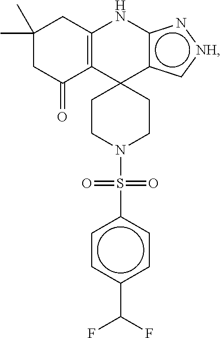 | 947248 |
| 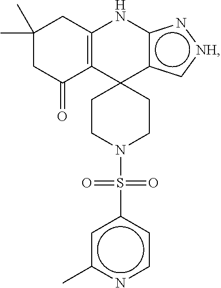 | 947481 |
| 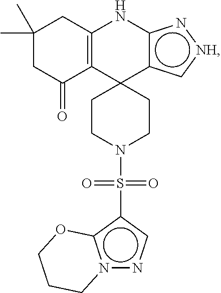 | 947483 |
| 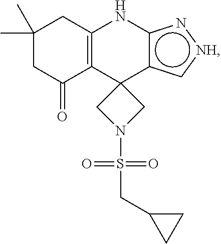 | 929440 |
| 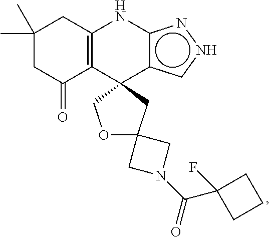 | 947735 |
| 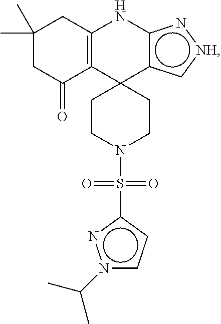 | 947258 |
| 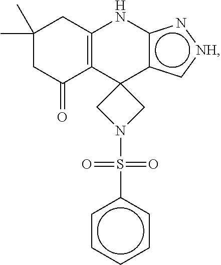 | 901474 |
| 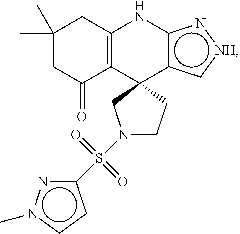 | 943484 |
| 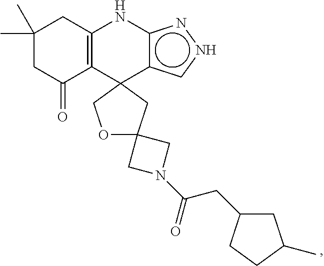 | 947269 |
| 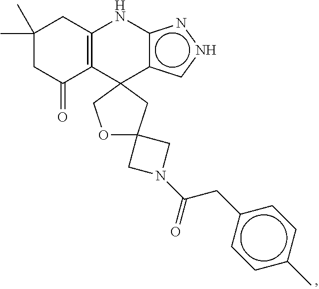 | 947281 |
| 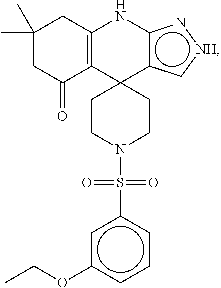 | 947253 |
| 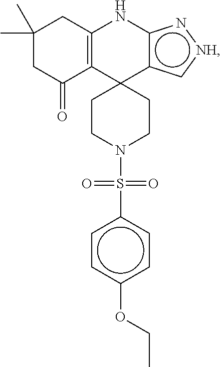 | 945670 |
| 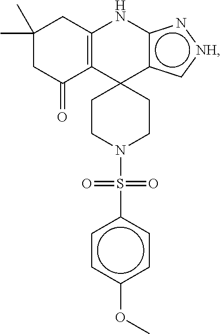 | 901469 |
| 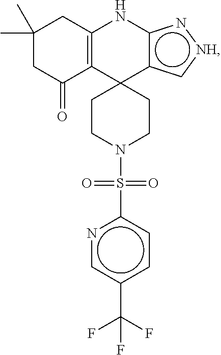 | 947902 |
| 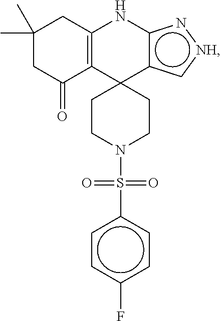 | 893311 |
| 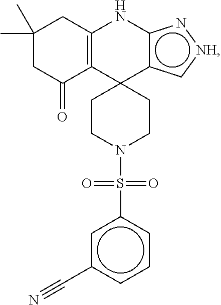 | 947254 |
| 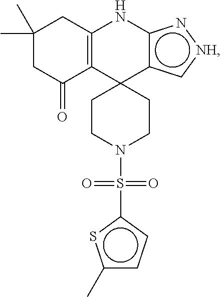 | 945672 |
| 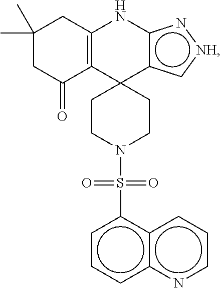 | 947469 |
| 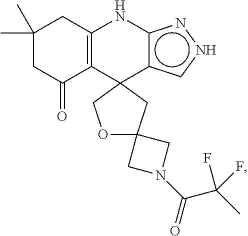 | 947275 |
| 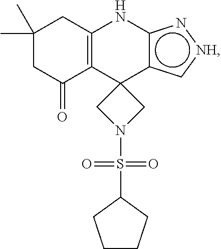 | 917139 |
| 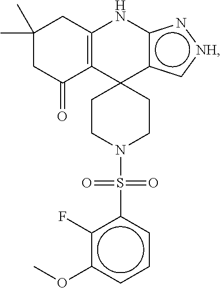 | 945656 |
| 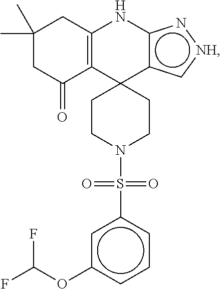 | 947242 |
| 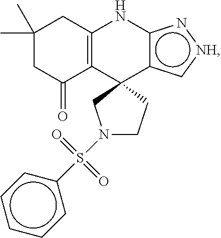 | 943485 |
| 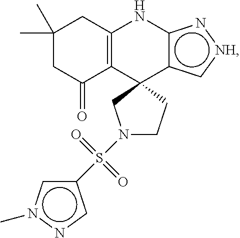 | 951725 |
| 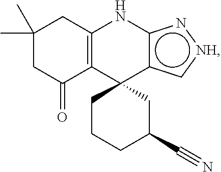 | 945805 |
| 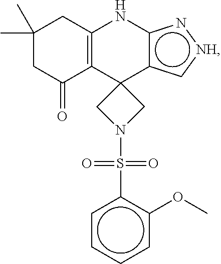 | 913261 |
| 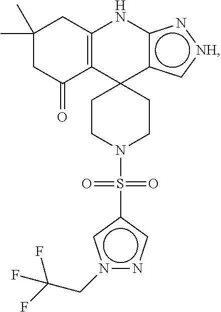 | 948123 |
| 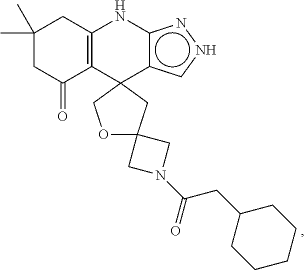 | 947305 |
| 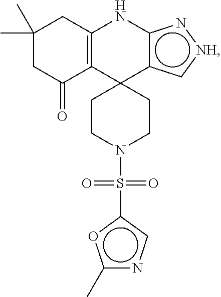 | 947611 |
| 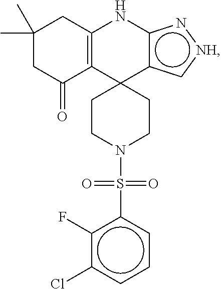 | 948118 |
| 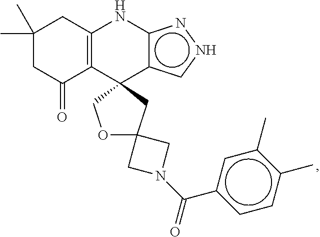 | 948038 |
| 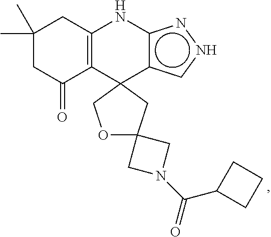 | 947288 |
| 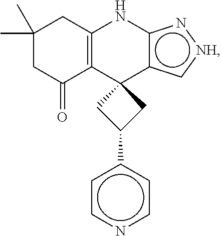 | 889665 |
| 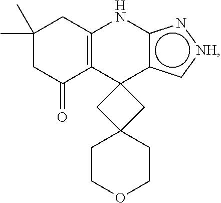 | 874534 |
| 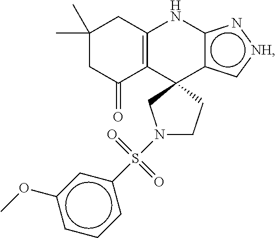 | 951789 |
| 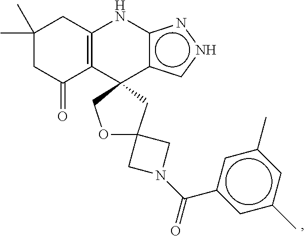 | 948223 |
| 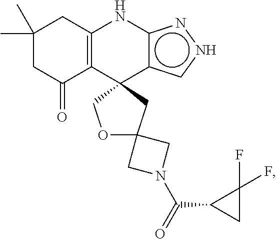 | 948215 |
| 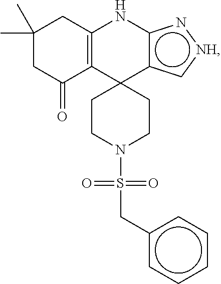 | 929437 |
| 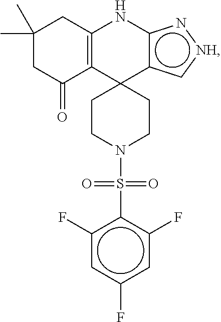 | 947484 |
| 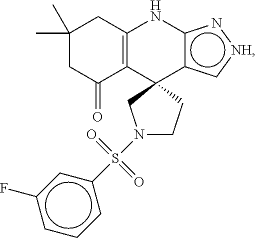 | 951861 |
| 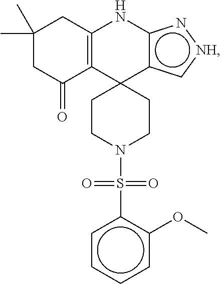 | 893310 |
| 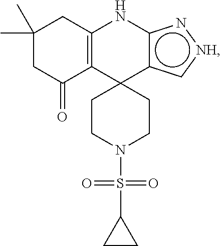 | 901470 |
| 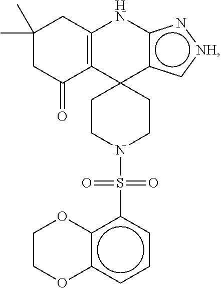 | 947464 |
| 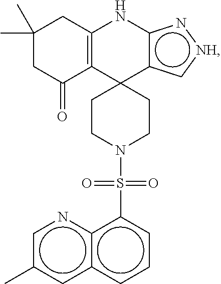 | 947614 |
| 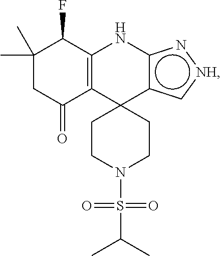 | 951701 |
| 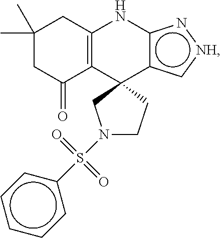 | 951787 |
| 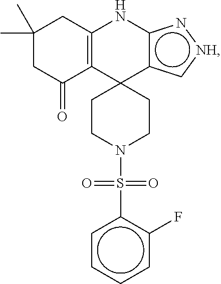 | 901465 |
| 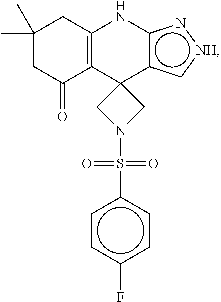 | 917144 |
| 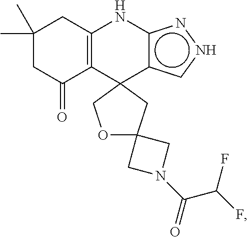 | 947485 |
| 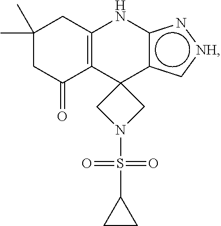 | 913259 |
| 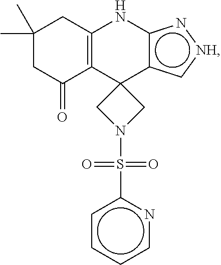 | 913262 |
| 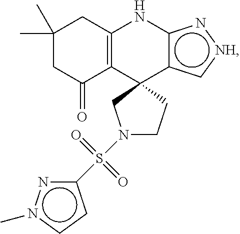 | 945712 |
| 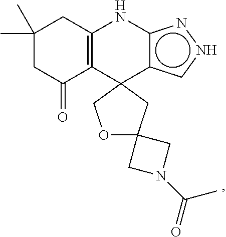 | 913058 |
| 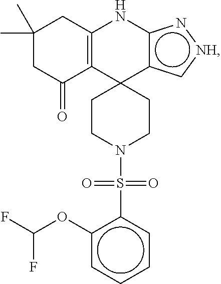 | 945668 |
| 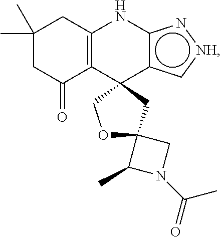 | 948440 |
| 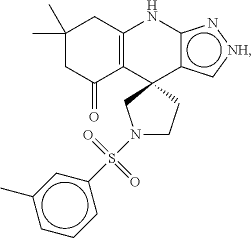 | 951863 |
| 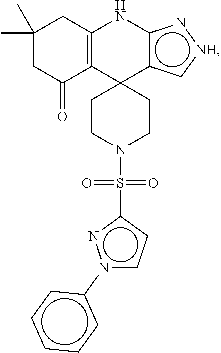 | 947461 |
| 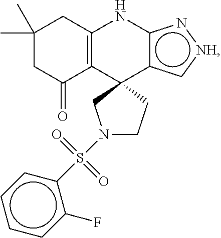 | 943487 |
| 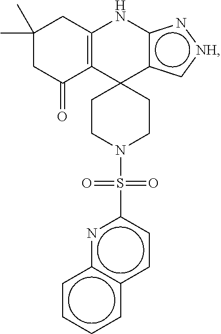 | 947476 |
| 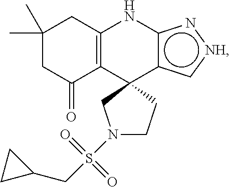 | 951723 |
| 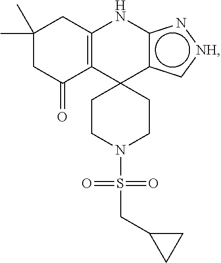 | 909207 |
| 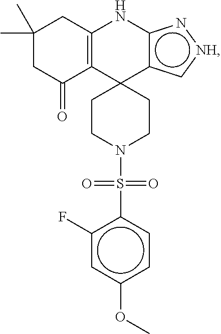 | 947255 |
| 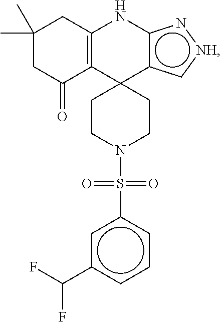 | 947264 |
| 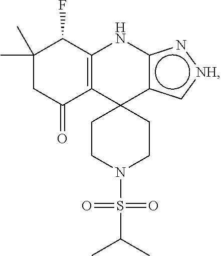 | 951714 |
| 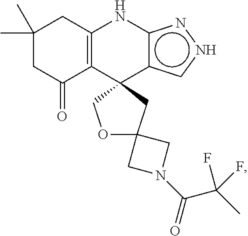 | 947838 |
| 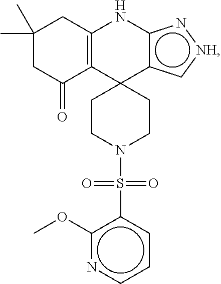 | 947260 |
| 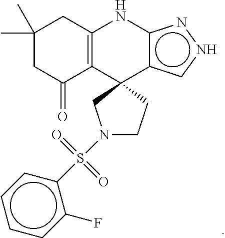 | 945718 |

In some embodiments, Formula III-A or III-B is any one of the formulae shown in Table 5A

TABLE 5A

| | |
|---|---|
| 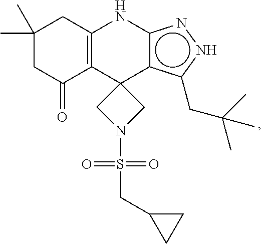 | 90285 |
| 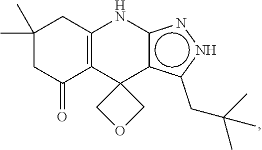 | 90709 |
| 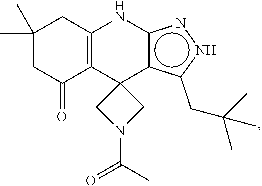 | 90991 |
| 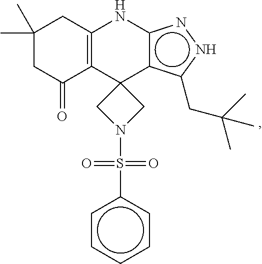 | 92737 |
| 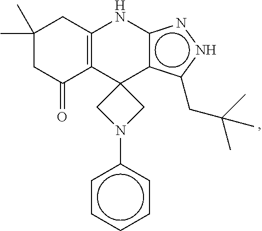 | 93429 |
| 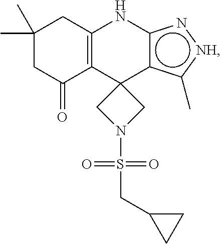 | 107828 |
| 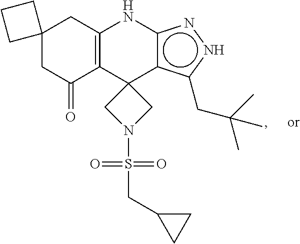 | 107829 |
| 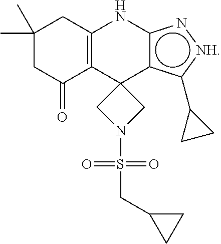 | 109152 |

In some embodiments, Formula III-A or III-B is any one of the formulae shown in Table 6:

TABLE 6

| | |
|---|---|
| 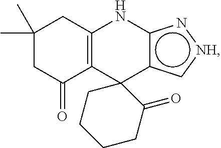 | 912105 |
| 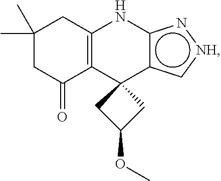 | 901472 |
| 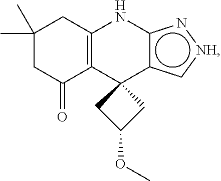 | 901473 |
| 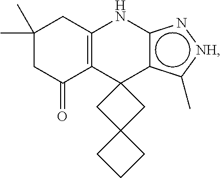 | 877295 |
| 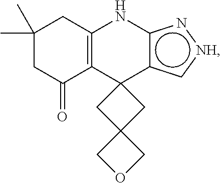 | 912168 |
| 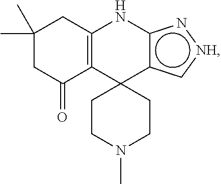 | 826714 |
| 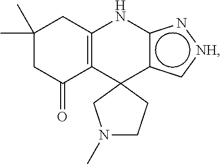 | 841867 |
| 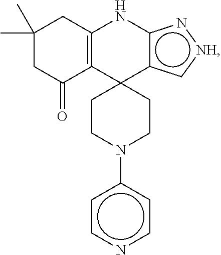 | 880765 |
| 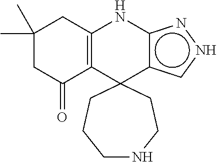 | 889571 |
| 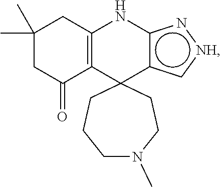 | 893313 |
| 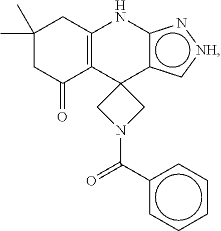 | 893315 |
| 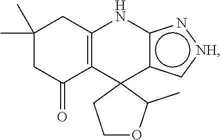 | 911958 |
| 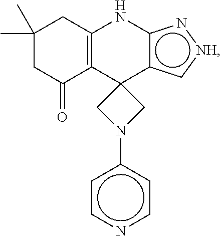 | 912077 |
| 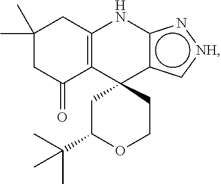 | 912107 |
| 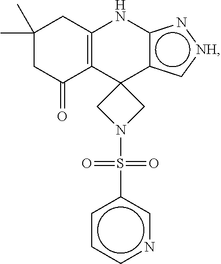 | 913265 |
| 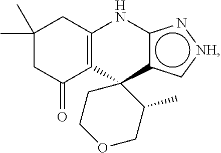 | 924310 |
| 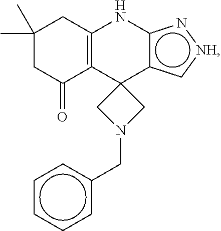 | 929436 |
| 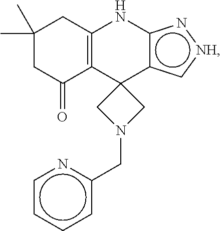 | 929438 |
| 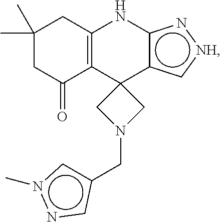 | 929439 |
| 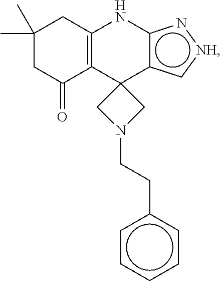 | 929441 |
| 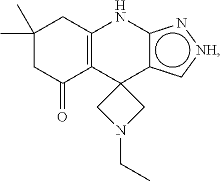 | 929448 |
| 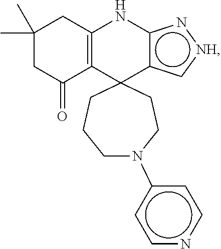 | 932633 |
| 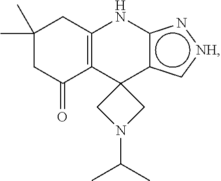 | 932650 |
| 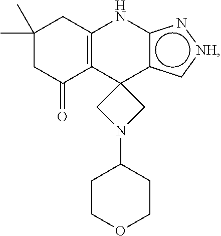 | 932651 |
| 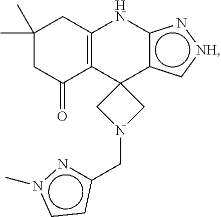 | 932652 |
| 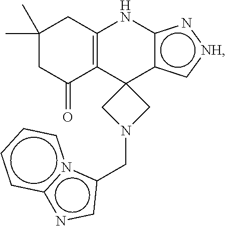 | 932654 |
| 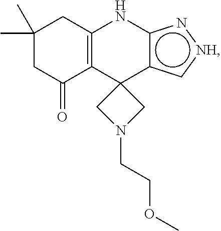 | 943544 |
| 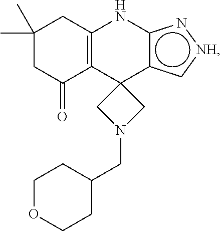 | 943545 |
| 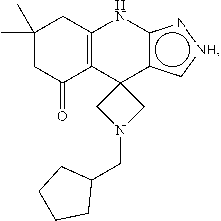 | 943914 |

In some embodiments, Formula III-A or III-B is any one of the formulae shown in Table 6A:

TABLE 6A

| 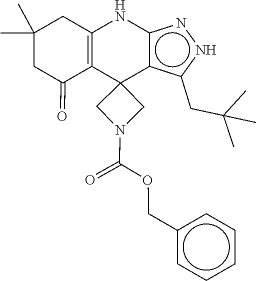 | 90330 |
|---|---|
| or | |
| 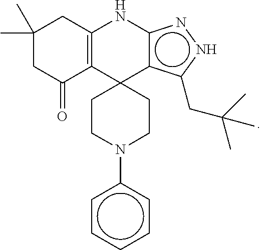 | 92315 |

In some embodiments, Formula III-A or III-B is not any one of the formulae shown in Table 6. In some embodiments, Formula III-A or III-B is not any one of the formulae shown in Table 6A.

In another aspect, provided herein is a compound, or a pharmaceutically acceptable salt, solvate, hydrate, polymorph, co-crystal, tautomer, stereoisomer, isotopically labeled compound, or prodrug thereof, wherein the compound is of Formula IV-A or IV-B.

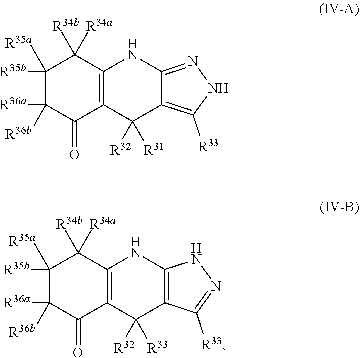

wherein:

R³¹ is

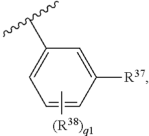

optionally substituted naphthyl, optionally substituted furanyl, optionally substituted thiophenyl, optionally substituted pyrrolyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, optionally substituted tetrazolyl, optionally substituted pyridinyl, optionally substituted pyrimidinyl, optionally substituted pyrazinyl, optionally substituted pyridazinyl, optionally substituted triazinyl, optionally substituted tetrazinyl, optionally substituted bicyclic heteroaryl, optionally substituted carbocyclyl, or optionally substituted heterocyclyl;
R³² is optionally substituted alkyl, optionally substituted alkenyl, or optionally substituted alkynyl;
each of R³³ and R³⁸ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(RDB, —OS(R^{D})OR^{D}, —OS(R^{D})(OR^{D})₂, or —OS(OR^{D})₃;
each of R^{34a}, R^{34b}, R^{35a}, R^{35b}, R^{36a}, and R^{36b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each instance of R^{D} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{D} attached to the same intervening atom are joined together with the intervening atom to form an optionally substituted, monocyclic, heterocyclic or heteroaryl ring;
or R^{34a} and R^{34b} are taken together with their intervening atom to form an optionally substituted, monocyclic, carbocyclic or heterocyclic ring;
or R^{34b} and R^{35a} are taken together with their intervening atom to form an optionally substituted, monocyclic, carbocyclic or heterocyclic ring;
or R^{35a} and R^{35b} are taken together with their intervening atom to form an optionally substituted, monocyclic, carbocyclic or heterocyclic ring;
or R^{35b} and R^{36a} are taken together with their intervening atom to form an optionally substituted, monocyclic, carbocyclic or heterocyclic ring; and
or R^{36a} and R^{36b} are taken together with their intervening atom to form an optionally substituted, monocyclic, carbocyclic or heterocyclic ring;
q1 is 0, 1, 2, 3, or 4; and
R³⁷ is —Br, —I, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃;
provided that R³⁷ is not-C(CH₃)₃, —CF₃, —CN, or —C(═O) NH₂.

In some embodiments, each of Formulae IV-A and IV-B is not

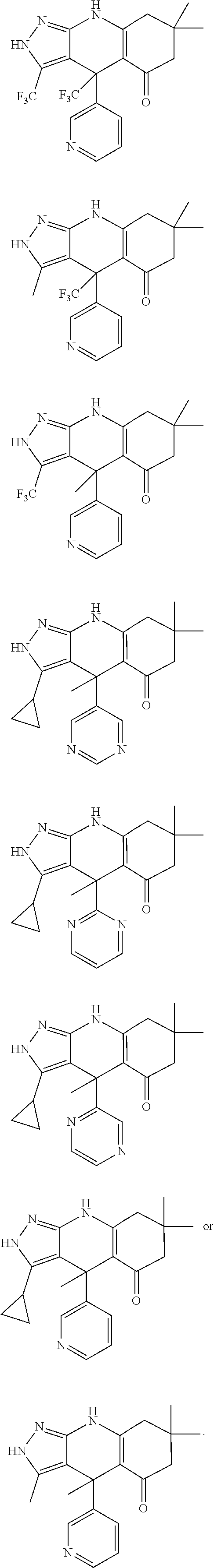

In some embodiments, the compound is of the formula:

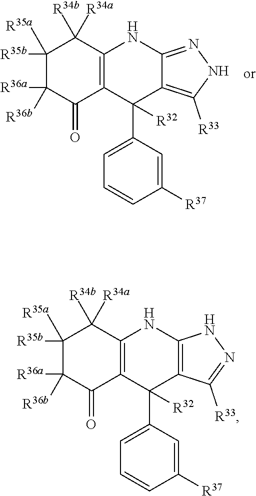

or a pharmaceutically acceptable salt, solvate, hydrate, polymorph, co-crystal, tautomer, stereoisomer, isotopically labeled compound, or prodrug thereof.

In some embodiments, the compound is of the formula:

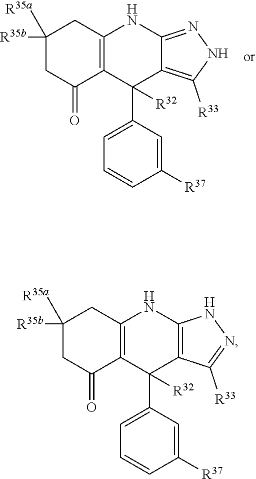

or a pharmaceutically acceptable salt, solvate, hydrate, polymorph, co-crystal, tautomer, stereoisomer, isotopically labeled compound, or prodrug thereof,
wherein:
R³⁷ is optionally substituted carbocyclyl, optionally substituted heterocyclyl, optionally substituted aryl, or optionally substituted heteroaryl.

In some embodiments, the compound is of the formula:

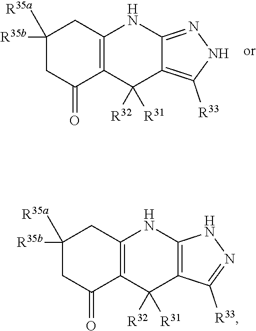

or a pharmaceutically acceptable salt, solvate, hydrate, polymorph, co-crystal, tautomer, stereoisomer, isotopically labeled compound, or prodrug thereof, wherein:
R³¹ is optionally substituted naphthyl, optionally substituted furanyl, optionally substituted thiophenyl, optionally substituted pyrrolyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, optionally substituted tetrazolyl, optionally substituted pyridinyl, optionally substituted pyrimidinyl, optionally substituted pyrazinyl, optionally substituted pyridazinyl, optionally substituted triazinyl, optionally substituted tetrazinyl, optionally substituted bicyclic heteroaryl, optionally substituted carbocyclyl, or optionally substituted heterocyclyl.

In some embodiments,

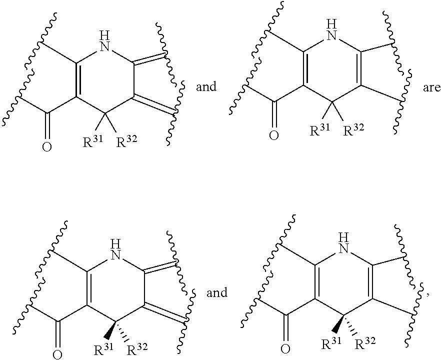

respectively. In some embodiments,

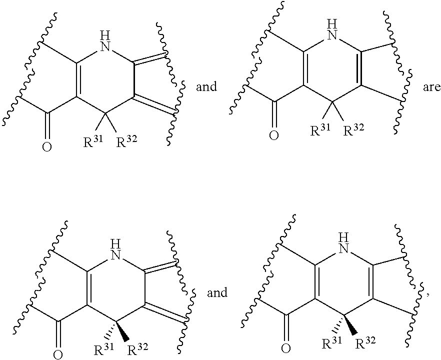

respectively.

In some embodiments, R³¹ is

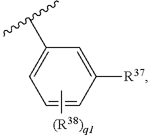

optionally substituted naphthyl, optionally substituted furanyl, optionally substituted thiophenyl, optionally substituted pyrrolyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, optionally substituted tetrazolyl, optionally substituted 2-pyridinyl, optionally substituted 4-pyridinyl, optionally substituted 4-pyrimidinyl, optionally substituted pyridazinyl, optionally substituted triazinyl, optionally substituted tetrazinyl, optionally substituted bicyclic heteroaryl, optionally substituted carbocyclyl, or optionally substituted heterocyclyl.

In some embodiments, R³¹ is optionally substituted furanyl, optionally substituted thiophenyl, optionally substituted pyrrolyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, optionally substituted tetrazolyl, optionally substituted 2-pyridinyl, optionally substituted pyridazinyl, optionally substituted triazinyl, or optionally substituted tetrazinyl. In some embodiments, R³¹ is optionally substituted furanyl, optionally substituted thiophenyl, or optionally substituted pyrrolyl. In some embodiments, R³¹ is optionally substituted imidazolyl, optionally substituted pyrazolyl, optionally substituted oxazolyl, optionally substituted isoxazolyl, optionally substituted thiazolyl, or optionally substituted isothiazolyl. In some embodiments, R³¹ is optionally substituted triazolyl, oxadiazolyl, or thiadiazolyl. In some embodiments, R³¹ is tetrazolyl. In some embodiments, R³¹ is optionally substituted pyridinyl, optionally substituted pyrimidinyl, optionally substituted pyrazinyl, optionally substituted pyridazinyl, optionally substituted triazinyl, or optionally substituted tetrazinyl. In some embodiments, R³¹ is optionally substituted 2-pyridinyl, optionally substituted 4-pyridinyl, optionally substituted 4-pyrimidinyl, optionally substituted pyridazinyl, optionally substituted triazinyl, or optionally substituted tetrazinyl. In some embodiments, R³¹ is optionally substituted 2-pyridinyl, optionally substituted pyridazinyl, optionally substituted triazinyl, or optionally substituted tetrazinyl.

In some embodiments, R³¹ is optionally substituted bicyclic heteroaryl. In some embodiments, R³¹ is optionally substituted bicyclic 8- to 14-membered heteroaryl.

In some embodiments, R³¹ is optionally substituted carbocyclyl or optionally substituted heterocyclyl. In some embodiments, R³¹ is optionally substituted C₃₋₁₄ carbocyclyl. In some embodiments, R³¹ is optionally substituted monocyclic C₃₋₇ carbocyclyl. In some embodiments, R³¹ is optionally substituted monocyclic C₃₋₄ carbocyclyl. In some embodiments, R³¹ is optionally substituted monocyclic C₅₋₇ carbocyclyl. In some embodiments, R³¹ is saturated carbocyclyl. In some embodiments, R³¹ is carbocyclyl comprising only one unsaturated bond (e.g., C═C bond) in the carbocyclic ring system.

In some embodiments, R³¹ is 3- to 14-membered optionally substituted heterocyclyl. In some embodiments, R³¹ is optionally substituted monocyclic 3- to 7-membered heterocyclyl. In some embodiments, R³¹ is optionally substituted 3- to 14-membered heterocyclyl comprising one or more O and/or S atoms but no N atoms. In some embodiments, R³¹ is optionally substituted 3-6 to 14-membered heterocyclyl comprising one or more N atoms and optionally one or more O and/S atoms. In some embodiments, R³¹ is optionally substituted monocyclic 3- to 7-membered heterocyclyl comprising one or more O and/or S atoms but no N atoms. In some embodiments, R³¹ is optionally substituted monocyclic 3- to 7-membered heterocyclyl comprising one or more N atoms and optionally one or more O and/or S atoms. In some embodiments, R³¹ is saturated heterocyclyl. In some embodiments, R³¹ is heterocyclyl comprising only one unsaturated bond (e.g., C═C bond) in the heterocyclic ring system.

In some embodiments, R³¹ is

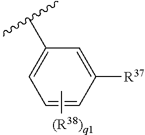

or optionally substituted naphthyl. In some embodiments, R³¹ is optionally substituted naphthyl. In some embodiments, R³¹ is

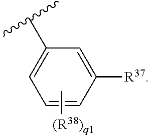

In some embodiments, R³¹ is

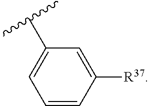

In some embodiments, R³¹ is

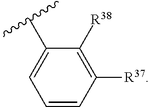

In some embodiments, R³¹ is

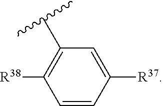

In some embodiments, R³¹ is

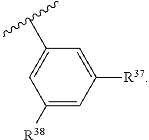

In some embodiments, R³¹ is

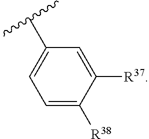

q1 is 0, 1, 2, 3, or 4. In some embodiments, q1 is 0, 1, 2, or 3. In some embodiments, q1 is 0, 1, or 2. In some embodiments, q1 is 0 or 1. In some embodiments, q1 is 0. In some embodiments, q1 is 1. In some embodiments, q1 is 2. In some embodiments, q1 is 3. In some embodiments, q1 is 4.

In some embodiments, at least one instance of R³⁸ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{D}, —SR^{D}, or —N(R^{D})₂. In some embodiments, at least one instance of R³⁸ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, at least one instance of R³⁸ is C₁₋₁₀ haloalkyl. In some embodiments, at least one instance of R³⁸ is C₁₋₄ haloalkyl. In some embodiments, at least one instance of R³⁸ is C₁₋₄ fluoroalkyl (e.g., C₁₋₄ perfluoroalkyl). In some embodiments, at least one instance of R³⁸ is —CF₃.

In some embodiments, at least one instance of R³⁸ is halogen. In some embodiments, at least one instance of R³⁸ is bromine, chlorine, or fluorine. In some embodiments, at least one instance of R³⁸ is bromine or chlorine. In some embodiments, at least one instance of R³⁸ is chlorine or fluorine. In some embodiments, at least one instance of R³⁸ is bromine. In some embodiments, at least one instance of R³⁸ is chlorine. In some embodiments, at least one instance of R³⁸ is fluorine.

In some embodiments, at least one instance of R³⁸ is —OR^{D}, —SR^{D}, or —N(R^{D})₂ (e.g., wherein R^{D} is hydrogen or optionally substituted alkyl, or optionally substituted phenyl). In some embodiments, at least one instance of R³⁸ is —OH. In some embodiments, at least one instance of R³⁸ is —SH. In some embodiments, at least one instance of R³⁸ is —NH₂. In some embodiments, at least one instance of R³⁸ is —CN, —SCN, —SSR^{D}, —N₃, —NO, or —NO₂. In some embodiments, at least one instance of R³⁸ is —C(═O)R^{D}, —C(═O)OR^{D}, —C(═O)SR^{D}, or —C(═O)N(R^{D})₂ (e.g., wherein R^{D} is hydrogen or optionally substituted alkyl, or optionally substituted phenyl). In some embodiments, at least one instance of R³⁸ is —C(═NR^{D})R^{D}, —C(═NR^{D})OR^{D}, —C(═NR^{D})SR^{D}, or —C(═NR^{D})N(R^{D})₂ (e.g., wherein R^{D} is hydrogen or optionally substituted alkyl, or optionally substituted phenyl). In some embodiments, at least one instance of R³⁸ is —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, or —S(═O)₂N(R^{D})₂ (e.g., wherein R^{D} is hydrogen or optionally substituted alkyl, or optionally substituted phenyl). In some embodiments, at least one instance of R³⁸ is —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)RP, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, or —ON(R^{D})₂ (e.g., wherein R^{D} is hydrogen or optionally substituted alkyl, or optionally substituted phenyl). In some embodiments, at least one instance of R³⁸ is —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, or —SC(═NR^{D})N(R^{D})₂ (e.g., wherein R^{D} is hydrogen or optionally substituted alkyl, or optionally substituted phenyl). In some embodiments, at least one instance of R³⁸ is —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, or —NR^{D}S(═O)₂N(R^{D})₂ (e.g., wherein R^{D} is hydrogen or optionally substituted alkyl, or optionally substituted phenyl). In some embodiments, at least one instance of R³⁸ is —Si(R^{D})₃, —Si(R^{D})₂OR^{D}, —Si(R^{D})(OR^{D})₂, —Si(OR^{D})₃, —OS(R^{D})₃, —OSi(R^{D})₂OR^{D}, —OSi(R^{D})(OR^{D})₂, or —OSi(OR^{D})₃ (e.g., wherein R^{D} is hydrogen or optionally substituted alkyl, or optionally substituted phenyl).

In some embodiments, at least one instance of R³⁸ is optionally substituted carbocyclyl, optionally substituted heterocyclyl, optionally substituted aryl, or optionally substituted heteroaryl. In some embodiments, at least one instance of R³⁸ is optionally substituted C₃₋₁₄ carbocyclyl. In some embodiments, at least one instance of R³⁸ is optionally substituted monocyclic C₃₋₇ carbocyclyl. In some embodiments, at least one instance of R³⁸ is optionally substituted monocyclic C₃₋₄ carbocyclyl. In some embodiments, at least one instance of R³⁸ is optionally substituted monocyclic C₃₋₇ carbocyclyl. In some embodiments, at least one instance of R³⁸ is saturated carbocyclyl. In some embodiments, at least one instance of R³⁸ is carbocyclyl comprising only one unsaturated bond (e.g., C═C bond) in the carbocyclic ring system.

In some embodiments, at least one instance of R³⁸ is optionally substituted 3- to 14-membered heterocyclyl. In some embodiments, at least one instance of R³⁸ is optionally substituted monocyclic 3- to 7-membered heterocyclyl. In some embodiments, at least one instance of R³⁸ is optionally substituted 3- to 14-membered heterocyclyl comprising one or more O and/or S atoms but no N atoms. In some embodiments, at least one instance of R³⁸ is optionally substituted 3- to 14-membered heterocyclyl comprising one or more N atoms and optionally one or more O and/S atoms. In some embodiments, at least one instance of R³⁸ is optionally substituted monocyclic 3- to 7-membered heterocyclyl comprising one or more O and/or S atoms but no N atoms. In some embodiments, at least one instance of R³⁸ is optionally substituted monocyclic 3- to 7-membered heterocyclyl comprising one or more N atoms and optionally one or more O and/or S atoms. In some embodiments, at least one instance of R³⁸ is saturated heterocyclyl. In some embodiments, at least one instance of R³⁸ is heterocyclyl comprising only one unsaturated bond (e.g., C═C bond) in the heterocyclic ring system.

In some embodiments, at least one instance of R³⁸ is optionally substituted monocyclic aryl. In some embodiments, at least one instance of R³⁸ is optionally substituted bicyclic aryl. In some embodiments, at least one instance of R³⁸ is optionally substituted C₆₋₁₄ aryl. In some embodiments, at least one instance of R³⁸ is optionally substituted C₆₋₁₀ aryl. In some embodiments, at least one instance of R³⁸ is optionally substituted phenyl. In some embodiments, at least one instance of R³⁸ is optionally substituted naphthyl.

In some embodiments, at least one instance of R³⁸ is optionally substituted monocyclic heteroaryl. In some embodiments, at least one instance of R³⁸ is optionally substituted bicyclic heteroaryl. In some embodiments, at least one instance of R³⁸ is optionally substituted 5- to 14-membered heteroaryl. In some embodiments, at least one instance of R³⁸ is optionally substituted 5- to 10-membered heteroaryl. In some embodiments, at least one instance of R³⁸ is optionally substituted 5- to 6-membered monocyclic heteroaryl. In some embodiments, at least one instance of R³⁸ is optionally substituted heteroaryl comprising one or more N atoms.

In some embodiments, R³⁷ is —Br, optionally substituted alkyl, optionally substituted heteroalkyl, optionally substituted carbocyclyl, optionally substituted heterocyclyl, optionally substituted aryl, optionally substituted heteroaryl, —OR^{D}, —SR^{D}, —N(R^{D})₂, —S(═O)₂R^{D}, —NR^{D}C(═O)R^{D}, or —NR^{D}S(═O)₂SR^{D}. In some embodiments, R³⁷ is —Br, —I, optionally substituted alkyl, optionally substituted alkenyl, optionally substituted alkynyl, optionally substituted heteroalkyl, optionally substituted heteroalkenyl, optionally substituted heteroalkynyl, —OR^{D}, —SR^{D}, or —N(R^{D})₂. In some embodiments, R³⁷ is optionally substituted alkyl, optionally substituted alkenyl, optionally substituted alkynyl, optionally substituted heteroalkyl, optionally substituted heteroalkenyl, optionally substituted heteroalkynyl, —OR^{D}, —SR^{D}, or —N(R^{D})₂. In some embodiments, R³⁷ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl.

In some embodiments, R³⁷ is —Br or —I. In some embodiments, R³⁷ is —Br. In some embodiments, R³⁷ is —I.

In some embodiments, R³⁷ is —OR^{D}, —SR^{D}, or —N(R^{D})₂ (e.g., wherein R^{D} is hydrogen or optionally substituted alkyl, or optionally substituted phenyl). In some embodiments, R³⁷ is —OH. In some embodiments, R³⁷ is —SH. In some embodiments, R³⁷ is —NH₂. In some embodiments, R³⁷ is —SCN, —SSR^{D}, —N₃, —NO, or —NO₂. In some embodiments, R³⁷ is —SSR^{D}, —N₃, —NO, or —NO₂. In some embodiments, R³⁷ is —C(═O)R^{D}, —C(═O)OR^{D}, —C(═O)SR^{D}, or —C(═O)N(R^{D})₂ (e.g., wherein R^{D} is hydrogen or optionally substituted alkyl, or optionally substituted phenyl). In some embodiments, R³⁷ is —C(═NR^{D})R^{D}, —C(═NR^{D})OR^{D}, —C(═NR^{D})SR^{D}, or —C(═NR^{D})N(R^{D})₂ (e.g., wherein R^{D} is hydrogen or optionally substituted alkyl, or optionally substituted phenyl). In some embodiments, R³⁷ is —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, or —S(═O)₂N(R^{D})₂ (e.g., wherein R^{D} is hydrogen or optionally substituted alkyl, or optionally substituted phenyl). In some embodiments, R³⁷ is —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)RP, —OS(═O)OR^{D}, —OS(═O)SR^{D}, —OS(═O)N(R^{D})₂, —OS(═O)₂R^{D}, —OS(═O)OR^{D}, —OS(═O)₂SR^{D}, —OS(═O) ZN(R^{D})₂, or —ON(R^{D})₂ (e.g., wherein R^{D} is hydrogen or optionally substituted alkyl, or optionally substituted phenyl). In some embodiments, R³⁷ is —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, or —SC(═NR^{D})N(R^{D})₂ (e.g., wherein R^{D} is hydrogen or optionally substituted alkyl, or optionally substituted phenyl). In some embodiments, R³⁷ is —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, or —NR^{D}S(═O)₂N(R^{D})₂ (e.g., wherein R^{D} is hydrogen or optionally substituted alkyl, or optionally substituted phenyl). In some embodiments, R³⁷ is —Si(R^{D})₃, —Si(R^{D})OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂ or —OSi(OR^{D})₃ (e.g., wherein R^{D} is hydrogen or optionally substituted alkyl, or optionally substituted phenyl).

In some embodiments, R³⁷ is optionally substituted carbocyclyl, optionally substituted heterocyclyl, optionally substituted aryl, or optionally substituted heteroaryl. In some embodiments, R³⁷ is optionally substituted carbocyclyl. In some embodiments, R³⁷ is optionally substituted C₃₋₁₄ carbocyclyl. In some embodiments, R³⁷ is optionally substituted monocyclic C₃₋₇ carbocyclyl. In some embodiments, R³⁷ is optionally substituted monocyclic C₃₋₄ carbocyclyl. In some embodiments, R³⁷ is optionally substituted monocyclic C₅₋₇ carbocyclyl. In some embodiments, R³⁷ is saturated carbocyclyl. In some embodiments, R³⁷ is carbocyclyl comprising only one unsaturated bond (e.g., C═C bond) in the carbocyclic ring system.

In some embodiments, R³⁷ is optionally substituted heterocyclyl. In some embodiments, R³⁷ is optionally substituted 3- to 14-membered heterocyclyl. In some embodiments, R³⁷ is optionally substituted monocyclic 3- to 7-membered heterocyclyl. In some embodiments, R³⁷ is optionally substituted 3- to 14-membered heterocyclyl comprising one or more O and/or S atoms but no N atoms. In some embodiments, R³⁷ is optionally substituted 3- to 14-membered heterocyclyl comprising one or more N atoms and optionally one or more O and/S atoms. In some embodiments, R³⁷ is optionally substituted monocyclic 3- to 7-membered heterocyclyl comprising one or more O and/or S atoms but no N atoms. In some embodiments, R³⁷ is optionally substituted monocyclic 3- to 7-membered heterocyclyl comprising one or more N atoms and optionally one or more O and/or S atoms. In some embodiments, R³⁷ is saturated heterocyclyl. In some embodiments, R³⁷ is heterocyclyl comprising only one unsaturated bond (e.g., C═C bond) in the heterocyclic ring system.

In some embodiments, R³⁷ is optionally substituted monocyclic aryl. In some embodiments, R³⁷ is optionally substituted bicyclic aryl. In some embodiments, R³⁷ is optionally substituted C₆₋₁₄ aryl. In some embodiments, R³⁷ is optionally substituted C₆₋₁₀ aryl. In some embodiments, R³⁷ is optionally substituted phenyl. In some embodiments, R³⁷ is optionally substituted naphthyl.

In some embodiments, R³⁷ is optionally substituted monocyclic heteroaryl. In some embodiments, R³⁷ is optionally substituted bicyclic heteroaryl. In some embodiments, R³⁷ is optionally substituted 5- to 14-membered heteroaryl. In some embodiments, R³⁷ is optionally substituted 5- to 10-membered heteroaryl. In some embodiments, R³⁷ is optionally substituted 5- to 6-membered monocyclic heteroaryl. In some embodiments, R³⁷ is optionally substituted heteroaryl comprising one or more N atoms. In some embodiments, R³⁷ is optionally substituted pyridinyl, optionally substituted isoquinolinyl, optionally substituted quinolinyl, or optionally substituted pyrazolyl. In some embodiments, R³⁷ is optionally substituted pyridinyl or optionally substituted pyrazolyl.

In some embodiments, R³² is optionally substituted alkyl, optionally substituted alkenyl, or optionally substituted alkynyl. In some embodiments, R³² is optionally substituted C₁₋₄ alkyl, optionally substituted C₁₋₄ alkenyl, or optionally substituted C₁₋₄ alkynyl. In some embodiments, R³² is optionally substituted alkyl. In some embodiments, R³² is optionally substituted C₁₋₄ alkyl. In some embodiments, R³² is unsubstituted C₁-C₄ alkyl. In some embodiments, R³² is unsubstituted methyl. In some embodiments, R³² is unsubstituted ethyl.

In some embodiments, R³³ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{D}, —SR^{D}, or —N(R^{D})₂. In some embodiments, R³³ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R³³ is C₁₋₁₀ haloalkyl. In some embodiments, R³³ is C₁₋₄ haloalkyl. In some embodiments, R³³ is C₁₋₄ fluoroalkyl (e.g., C₁₋₄ perfluoroalkyl). In some embodiments, R³³ is —CF₃. In some embodiments, R³³ is hydrogen, optionally substituted C₁-C₆ alkyl, or halogen. In some embodiments, R³³ is hydrogen, fluorine, —CH₃, —CH₂F, —CHF₂, or —CF₃. In some embodiments, R³³ is hydrogen, fluorine, —CH₃, or —CF₃. In some embodiments, R³³ is hydrogen or halogen. In some embodiments, R³³ is hydrogen or fluorine. In some embodiments, R³³ is hydrogen or optionally substituted C₁-C₆ alkyl. In some embodiments, R³³ is hydrogen, unsubstituted C₁-C₆ alkyl, or C₁₋₆ haloalkyl. In some embodiments, R³³ is hydrogen.

In some embodiments, R³³ is halogen. In some embodiments, R³³ is bromine, chlorine, or fluorine. In some embodiments, R³³ is bromine or chlorine. In some embodiments, R³³ is chlorine or fluorine. In some embodiments, R³³ is bromine. In some embodiments, R³³ is chlorine. In some embodiments, R³³ is fluorine.

In some embodiments, R³³ is —OR^{D}, —SR^{D}, or —N(R^{D})₂ (e.g., wherein R^{D} is hydrogen or optionally substituted alkyl, or optionally substituted phenyl). In some embodiments, R³³ is —OH. In some embodiments, R³³ is —SH. In some embodiments, R³³ is —NH₂. In some embodiments, R³³ is —CN, —SCN, —SSR^{D}, —N₃, —NO, or —NO₂. In some embodiments, R³³ is —C(═O)R^{D}, —C(═O)OR^{D}, —C(═O)SR^{D}, or —C(═O)N(R^{D})₂ (e.g., wherein R^{D} is hydrogen or optionally substituted alkyl, or optionally substituted phenyl). In some embodiments, R³³ is —C(═NR^{D})R^{D}, —C(═NR^{D})OR^{D}, —C(═NR^{D})SR^{D}, or —C(═NR^{D})N(R^{D})₂ (e.g., wherein R^{D} is hydrogen or optionally substituted alkyl, or optionally substituted phenyl). In some embodiments, R³³ is —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, or —S(═O)₂N(R^{D})₂ (e.g., wherein R^{D} is hydrogen or optionally substituted alkyl, or optionally substituted phenyl). In some embodiments, R³³ is —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, or —ON(R^{D})₂ (e.g., wherein R^{D} is hydrogen or optionally substituted alkyl, or optionally substituted phenyl). In some embodiments, R³³ is —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, or —SC(═NR^{D})N(R^{D}), (e.g., wherein R^{D} is hydrogen or optionally substituted alkyl, or optionally substituted phenyl). In some embodiments, R³³ is —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, or —NR^{D}S(═O)₂N(R^{D})₂ (e.g., wherein R^{D} is hydrogen or optionally substituted alkyl, or optionally substituted phenyl). In some embodiments, R³ is —Si(R^{D})₃, —Si(R^{D})₂OR^{D}, —Si(R^{D})(OR^{D})₂, —Si(OR^{D})₃, —OS(R^{D})₃, —OSi(R^{D})₂OR^{D}, —OSi(R^{D})(OR^{D})₂, or —OSi(OR^{D})₃ (e.g., wherein R^{D} is hydrogen or optionally substituted alkyl, or optionally substituted phenyl).

In some embodiments, R³³ is optionally substituted carbocyclyl, optionally substituted heterocyclyl, optionally substituted aryl, or optionally substituted heteroaryl. In some embodiments, R³³ is optionally substituted C₃₋₁₄ carbocyclyl. In some embodiments, R³³ is optionally substituted monocyclic C₃₋₇ carbocyclyl. In some embodiments, R³³ is optionally substituted monocyclic C₃₋₄ carbocyclyl. In some embodiments, R³³ is optionally substituted monocyclic C₅₋₇ carbocyclyl. In some embodiments, R³³ is saturated carbocyclyl. In some embodiments, R³³ is carbocyclyl comprising only one unsaturated bond (e.g., C═C bond) in the carbocyclic ring system.

In some embodiments, R³³ is optionally substituted 3- to 14-membered heterocyclyl. In some embodiments, R³³ is optionally substituted monocyclic 3- to 7-membered heterocyclyl. In some embodiments, R³³ is optionally substituted 3- to 14-membered heterocyclyl comprising one or more O and/or S atoms but no N atoms. In some embodiments, R³³ is optionally substituted 3- to 14-membered heterocyclyl comprising one or more N atoms and optionally one or more O and/S atoms. In some embodiments, R³³ is optionally substituted monocyclic 3- to 7-membered heterocyclyl comprising one or more O and/or S atoms but no N atoms. In some embodiments, R³³ is optionally substituted monocyclic 3- to 7-membered heterocyclyl comprising one or more N atoms and optionally one or more O and/or S atoms. In some embodiments, R³³ is saturated heterocyclyl. In some embodiments, R³³ is heterocyclyl comprising only one unsaturated bond (e.g., C═C bond) in the heterocyclic ring system.

In some embodiments, R³³ is optionally substituted monocyclic aryl. In some embodiments, R³³ is optionally substituted bicyclic aryl. In some embodiments, R³³ is optionally substituted C₆₋₁₄ aryl. In some embodiments, R³³ is optionally substituted C₆₋₁₀ aryl. In some embodiments, R³³ is optionally substituted phenyl. In some embodiments, R³³ is optionally substituted naphthyl.

In some embodiments, R³³ is optionally substituted monocyclic heteroaryl. In some embodiments, R³³ is optionally substituted bicyclic heteroaryl. In some embodiments, R³³ is optionally substituted 5- to 14-membered heteroaryl. In some embodiments, R³³ is optionally substituted 5- to 10-membered heteroaryl. In some embodiments, R³³ is optionally substituted 5- to 6-membered monocyclic heteroaryl.

In some embodiments, at least one instance of R^{D} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{D} is optionally substituted carbocyclyl, optionally substituted heterocyclyl, optionally substituted aryl, or optionally substituted heteroaryl. In some embodiments, at least one instance of R^{D} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{D} is independently hydrogen, optionally substituted C₁₋₁₀ alkyl, or optionally substituted phenyl. In some embodiments, at least one instance of R^{D} is a nitrogen protecting group when attached to a nitrogen atom, an oxygen protecting group when attached to an oxygen atom, or a sulfur protecting group when attached to a sulfur atom. In some embodiments, two instances of R^{D} attached to the same intervening atom are joined together with the intervening atom to form an optionally substituted, monocyclic, heterocyclic or heteroaryl ring.

In some embodiments, each of R^{34a}, R^{34b}, R^{35a}, R^{35b}, R^{36a}, and R^{36b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, each of R^{34a}, R^{34b}, R^{35a}, R^{35b}, R^{36a}, and R^{36b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, each of R^{34a}, R^{34b}, R^{35a}, R^{35b}, R^{36a}, and R^{36b} is independently hydrogen, halogen, or optionally substituted alkyl. In some embodiments, each of R^{34a}, R^{34b}, R^{35a}, R^{35b}, R^{36a}, and R^{36b} is independently hydrogen, halogen, or optionally substituted C₁₋₄ alkyl. In some embodiments, each of R^{34a}, R^{34b}, R^{35a}, R^{35b}, R^{36a}, and R^{36b} is independently hydrogen, fluorine, or optionally substituted C₁₋₄ alkyl. In some embodiments, each of R^{34a}, R^{34b}, R^{35a}, R^{35b}, R^{36a}, and R^{36b} is independently hydrogen or halogen. In some embodiments, each of R^{34a}, R^{34b}, R^{35a}, R^{35b}, R^{36a}, and R^{36b} is independently hydrogen or fluorine. In some embodiments, each of R^{34a}, R^{34b}, R^{35a}, R^{35b}, R^{36a}, and R^{36b} is independently hydrogen or optionally substituted C₁₋₄ alkyl. In some embodiments, at least one of R^{34a}, R^{34b}, R^{35a}, R^{35b}, R^{36a}, and R^{36b} is hydrogen. In some embodiments, each of R^{34a}, R^{34b}, R^{35a}, R^{35b}, R^{36a}, and R^{36b} is hydrogen. In some embodiments, at least one of R^{34a}, R^{34b}, R^{35a}, R^{35b}, R^{36a}, and R^{36b} is optionally substituted C₁₋₄ alkyl. In some embodiments, at least one of R^{34a}, R^{34b}, R^{35a}, R^{35b}b, R^{36a}, and R^{36b} is unsubstituted methyl. In some embodiments, at least one of R^{34a}, R^{34b}, R^{35a}, R^{35b}, R^{36a}, and R^{36b} is fluorine.

In some embodiments, each of R^{34a} and R^{34b} is independently hydrogen, halogen, or optionally substituted alkyl. In some embodiments, each of R^{34a} and R^{34b} is independently hydrogen, halogen, or optionally substituted C₁₋₄ alkyl. In some embodiments, at least one of R^{34a} and R^{34b} is halogen. In some embodiments, at least one of R^{34a} and R^{34b} is fluorine. In some embodiments, at least one of R^{34a} and R^{34b} is C₁₋₄ alkyl. In some embodiments, at least one of R^{34a} and R^{34b} is hydrogen. In some embodiments, R^{34a} and R^{34b} are hydrogen.

In some embodiments, each of R^{35a} and R^{35b} is independently hydrogen, halogen, or optionally substituted alkyl. In some embodiments, each of R^{35a} and R^{35b} is independently hydrogen, halogen, or optionally substituted C₁₋₄ alkyl. In some embodiments, at least one of R^{35a} and R^{35b} is halogen. In some embodiments, at least one of R^{35a} and R^{35b} is fluorine. In some embodiments, at least one of R^{35a} and R^{35b} is optionally substituted C₁₋₄ alkyl. In some embodiments, at least one of R^{35a} and R^{35b} is —CH₃. In some embodiments, R^{35a} and R^{35b} are optionally substituted C₁₋₄ alkyl. In some embodiments, R^{35a} and R^{35b} are —CH₃, In some embodiments, at least one of R^{35a} and R^{35b} is hydrogen. In some embodiments, R^{35a} and R^{35b} are hydrogen.

In some embodiments, each of R^{36a} and R^{36b} is independently hydrogen, halogen, or optionally substituted alkyl. In some embodiments, each of R^{36a} and R^{36b} is independently hydrogen, halogen, or optionally substituted C₁₋₄ alkyl. In some embodiments, at least one of R^{36a} and R^{36b} is halogen. In some embodiments, at least one of R^{36a} and R^{36b} is fluorine. In some embodiments, at least one of R^{36a} and R^{36b} is C₁₋₄ alkyl. In some embodiments, at least one of R^{36a} and R^{36b} is hydrogen. In some embodiments, R^{36a} and R^{36b} are hydrogen.

In some embodiments, R^{34a} and R^{34b} are taken together with their intervening atom to form an optionally substituted, monocyclic, carbocyclic or heterocyclic ring. In some embodiments, R^{34a} and R^{34b} are taken together with their intervening atom to form an optionally substituted, monocyclic, C₃₋₁₄ carbocyclic ring. In some embodiments, R^{34a} and R^{34b} are taken together with their intervening atom to form an optionally substituted, monocyclic, C₃₋₇ carbocyclic ring. In some embodiments, R^{34a} and R^{34b} are taken together with their intervening atom to form an optionally substituted, monocyclic, 3- to 14-membered heterocyclic ring. In some embodiments, R^{34a} and R^{34b} are taken together with their intervening atom to form an optionally substituted, monocyclic, 3- to 7-membered heterocyclic ring.

In some embodiments, R^{34b} and R^{35a} are taken together with their intervening atom to form an optionally substituted, monocyclic, carbocyclic or heterocyclic ring. In some embodiments, R^{34b} and R^{35a} are taken together with their intervening atom to form an optionally substituted, monocyclic, C₃₋₁₄ carbocyclic ring. In some embodiments, R^{34b} and R^{35a} are taken together with their intervening atom to form an optionally substituted, monocyclic, C₃₋₇ carbocyclic ring. In some embodiments, R^{34b} and R^{35a} are taken together with their intervening atom to form an optionally substituted, monocyclic, 3- to 14-membered heterocyclic ring. In some embodiments, R^{34b} and R^{35a} are taken together with their intervening atom to form an optionally substituted, monocyclic, 3- to 7-membered heterocyclic ring.

In some embodiments, R^{35a} and R^{35b} are taken together with their intervening atom to form an optionally substituted, monocyclic, carbocyclic or heterocyclic ring. In some embodiments, R^{35a} and R^{35b} are taken together with their intervening atom to form an optionally substituted, monocyclic, C₃₋₁₅ carbocyclic ring. In some embodiments, R^{35a} and R^{35b} are taken together with their intervening atom to form an optionally substituted, monocyclic, C₃₋₇ carbocyclic ring. In some embodiments, R^{35a} and R^{35b} are taken together with their intervening atom to form an optionally substituted, monocyclic, 3- to 15-membered heterocyclic ring. In some embodiments, R^{35a} and R^{35b} are taken together with their intervening atom to form an optionally subsituted, monocyclic, 3- to 7-membered heterocyclic ring.

In some embodiments, R^{35b} and R^{36a} are taken together with their intervening atom to form an optionally substituted, monocyclic, carbocyclic or heterocyclic ring. In some embodiments, R^{35b} and R^{36a} are taken together with their intervening atom to form an optionally substituted, monocyclic, C₃₋₁₄ carbocyclic ring. In some embodiments, R^{35b} and R^{36a} are taken together with their intervening atom to form an optionally substituted, monocyclic, C₃₋₇ carbocyclic ring. In some embodiments, R^{35b} and R^{36a} are taken together with their intervening atom to form an optionally substituted, monocyclic, 3- to 14-membered heterocyclic ring. In some embodiments, R^{35b} and R^{36a} are taken together with their intervening atom to form an optionally substituted, monocyclic, 3- to 7-membered heterocyclic ring.

In some embodiments, R^{36a} and R^{36b} are taken together with their intervening atom to form an optionally substituted, monocyclic, carbocyclic or heterocyclic ring. In some embodiments, R^{36a} and R^{36b} are taken together with their intervening atom to form an optionally substituted, monocyclic, C₃₋₁₅ carbocyclic ring. In some embodiments, R^{36a} and R^{36b} are taken together with their intervening atom to form an optionally substituted, monocyclic, C₃₋₇ carbocyclic ring. In some embodiments, R^{36a} and R^{36b} are taken together with their intervening atom to form an optionally substituted, monocyclic, 3- to 15-membered heterocyclic ring. In some embodiments, R^{36a} and R^{36b} are taken together with their intervening atom to form an optionally substituted, monocyclic, 3- to 7-membered heterocyclic ring.

In some embodiments, Formula IV-A or IV-B is

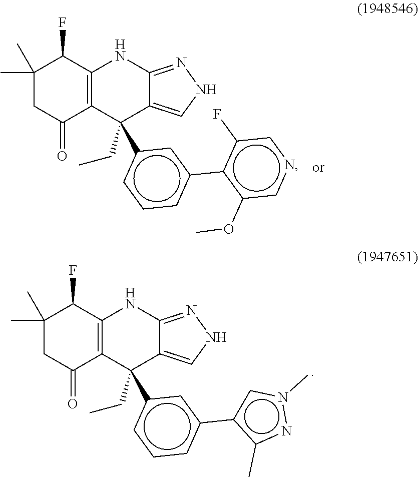

In some embodiments, Formula IV-A or IV-B is any one of the formulae shown in Table 7:

TABLE 7

| | |
|---|---|
| 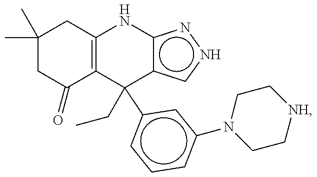 | 806604 |
| 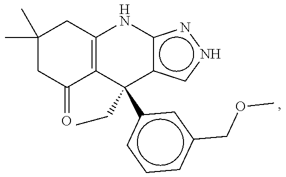 | 810362 |
| 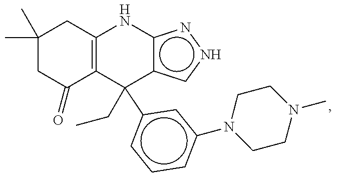 | 810379 |
| 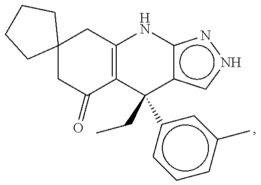 | 801313 |
| 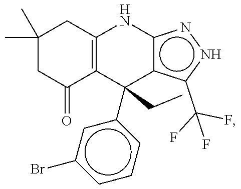 | 810365 |
| 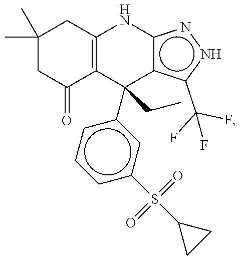 | 945713 |
| 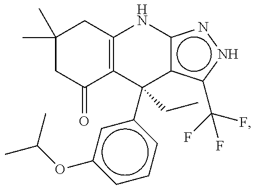 | 947867 |
| 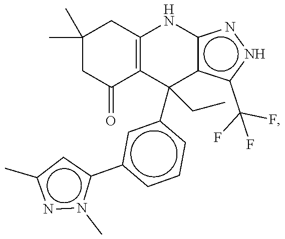 | 945532 |
| 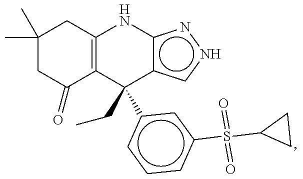 | 912171 |
| 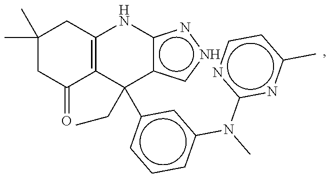 | 942642 |
| 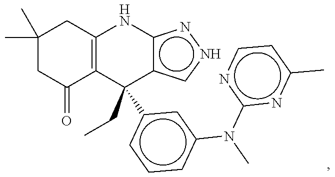 | 945328 |
| 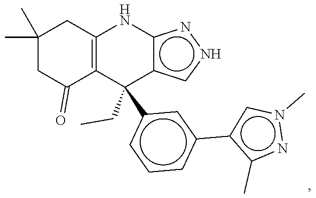 | 945546 |
| 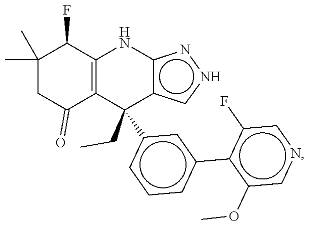 | 948545 |
| 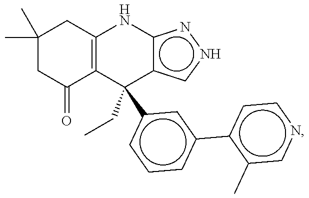 | 913047 |
| 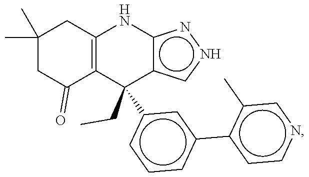 | 943990 |
| 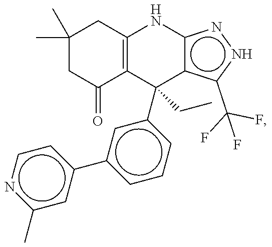 | 917291 |
| 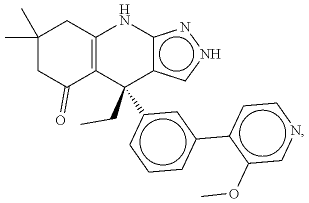 | 945481 |
| 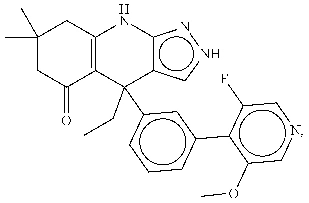 | 945704 |
| 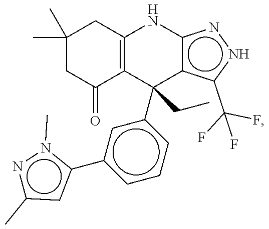 | 947384 |
| 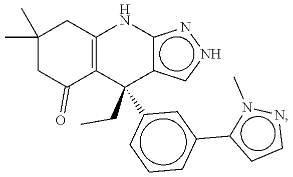 | 911992 |
| 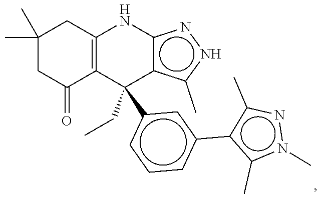 | 913043 |
| 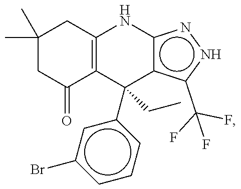 | 810366 |
| 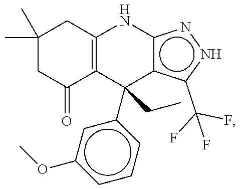 | 884117 |
| 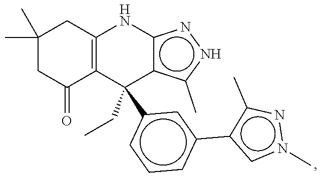 | 913035 |
| 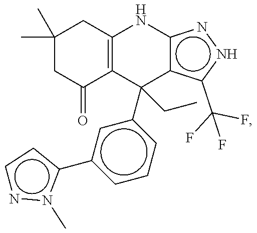 | 945531 |
| 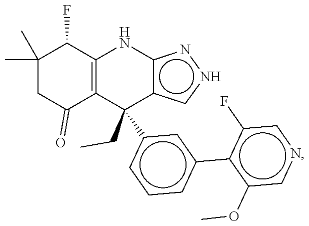 | 948546 |
| 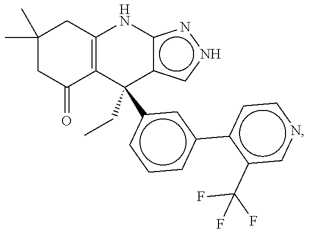 | 943913 |
| 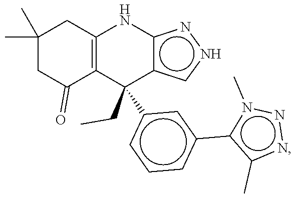 | 946099 |
| 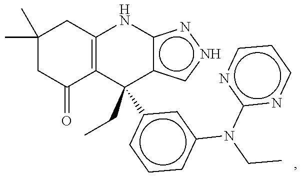 | 947333 |
| 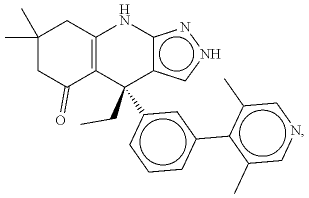 | 947658 |
| 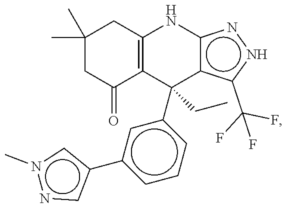 | 811565 |
| 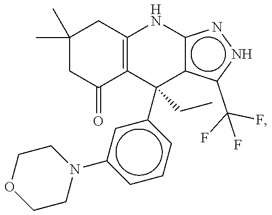 | 816885 |
| 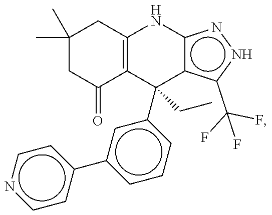 | 917293 |
| 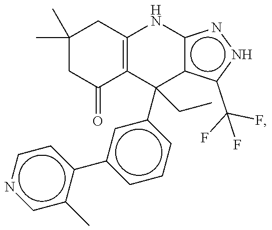 | 945527 |
| 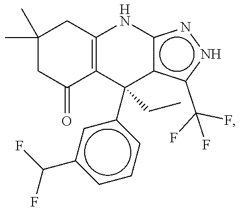 | 945710 |
| 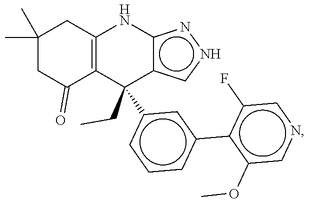 | 947511 |
| 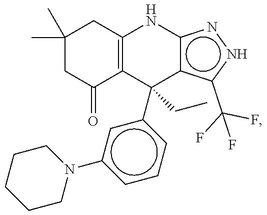 | 816800 |
| 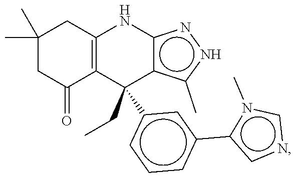 | 913032 |
| 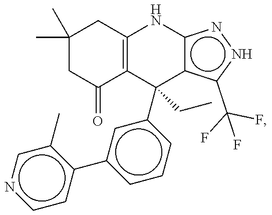 | 947324 |
| 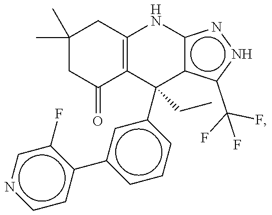 | 947326 |
| 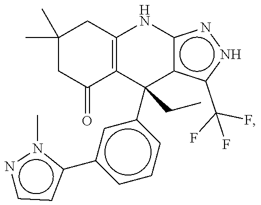 | 947372 |
| 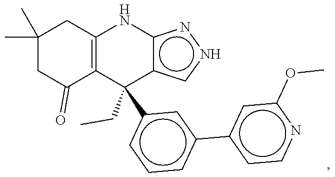 | 911989 |
| 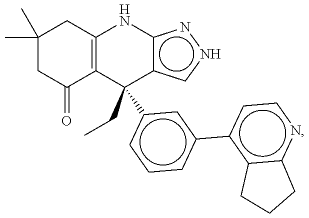 | 945619 |
| 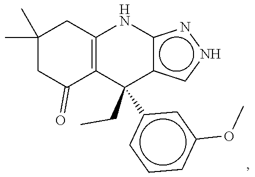 | 811561 |
| 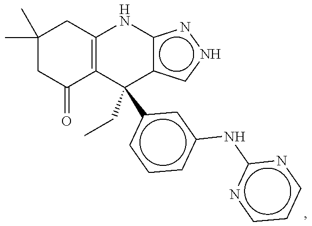 | 945958 |
| 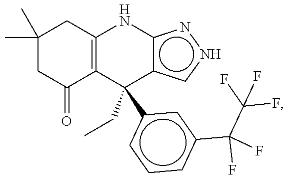 | 949268 |
| 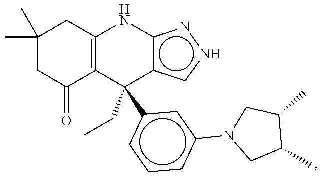 | 918327 |
| 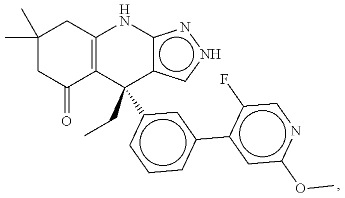 | 944035 |
| 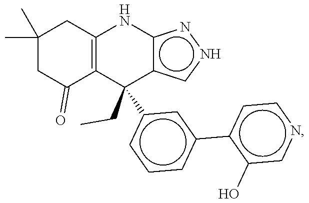 | 945728 |
| 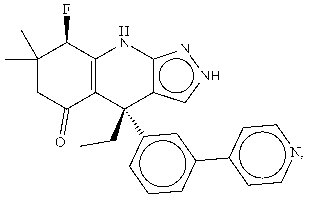 | 959342 |
| 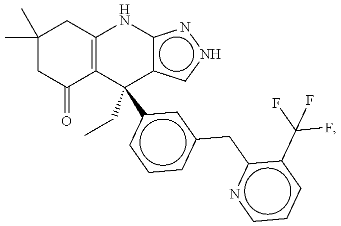 | 947665 |
| 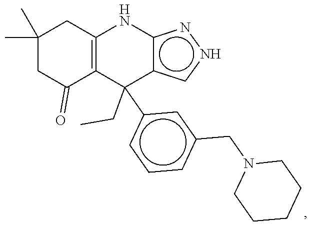 | 819560 |
| 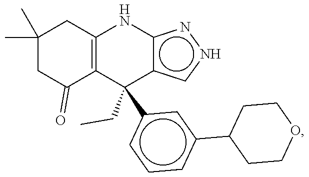 | 819639 |
| 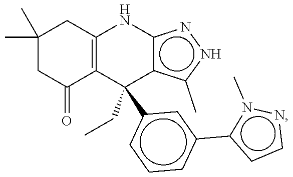 | 913046 |
| 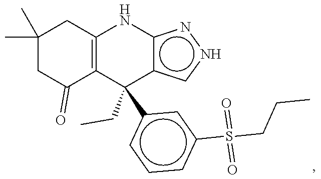 | 929452 |
| 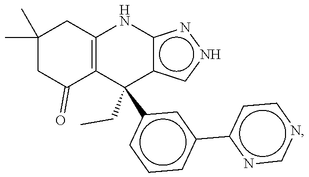 | 810489 |
| 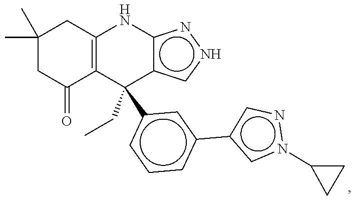 | 913041 |
| 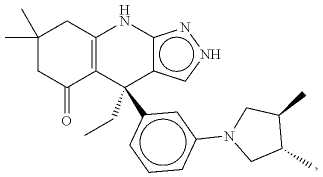 | 924543 |
| 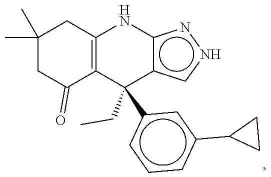 | 811558 |
| 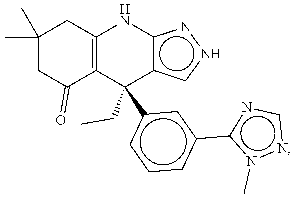 | 945482 |
| 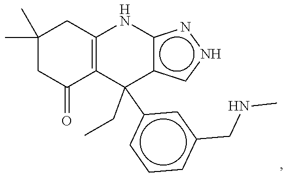 | 826651 |
| 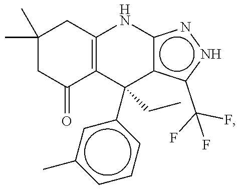 | 883872 |
| 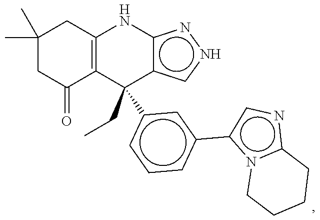 | 945618 |
| 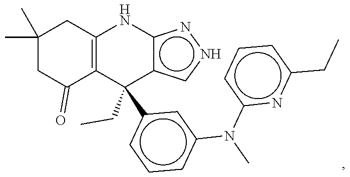 | 945726 |
| 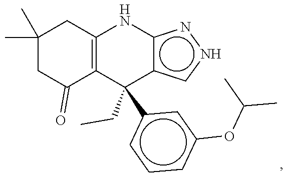 | 811563 |
| 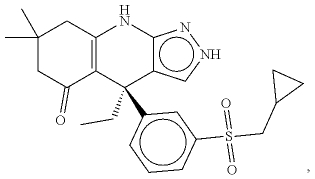 | 917151 |
| 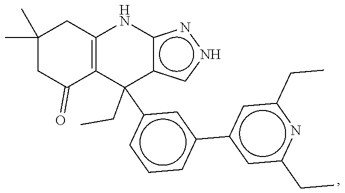 | 929445 |
| 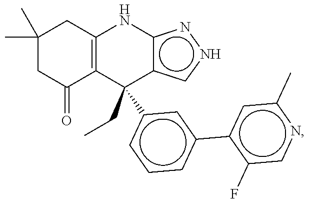 | 927339 |
| 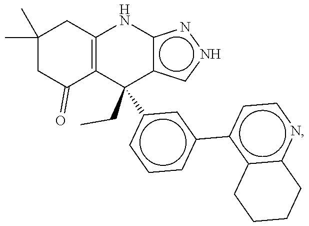 | 944036 |
| 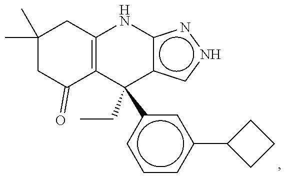 | 949514 |
| 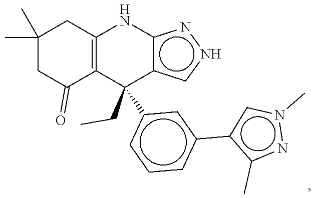 | 945545 |
| 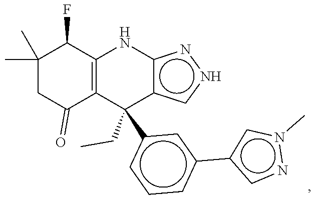 | 957510 |
| 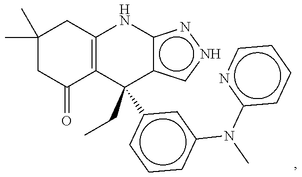 | 875608 |
| 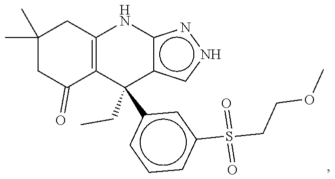 | 943546 |
| 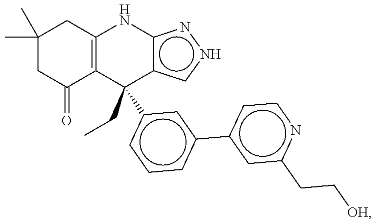 | 947816 |
| 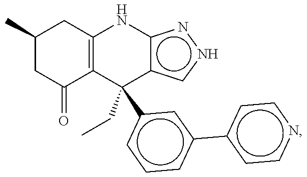 | 874315 |
| 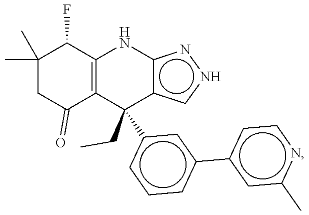 | 946101 |
| 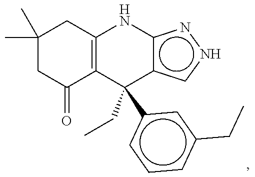 | 949391 |
| 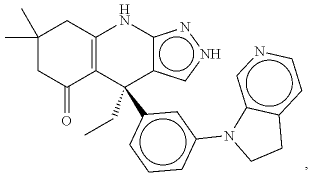 | 912004 |
| 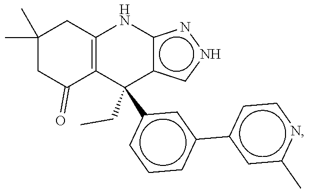 | 865170 |
| 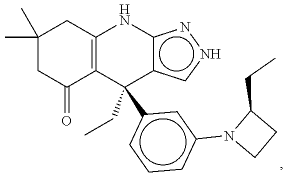 | 911972 |
| 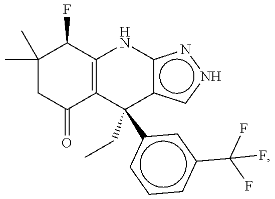 | 837803 |
| 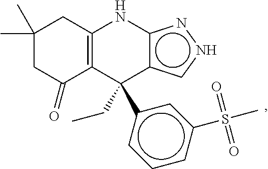 | 826719 |
| 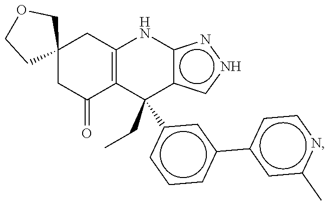 | 947412 |
| 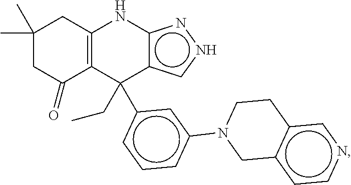 | 929449 |
| 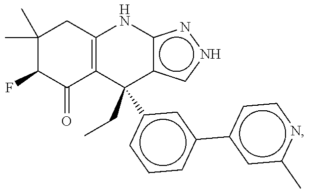 | 945486 |
| 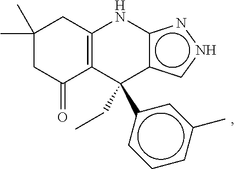 | 789561 |
| 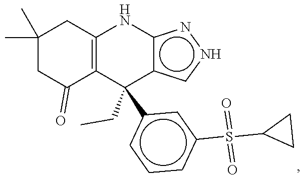 | 9121272 |
| 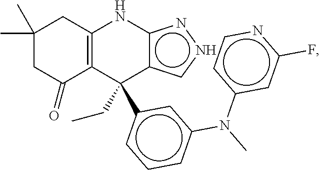 | 929453 |
| 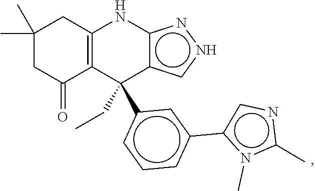 | 945612 |
| 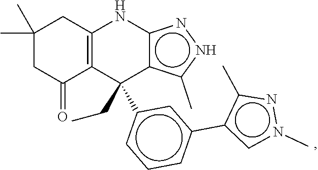 | 913034 |
| 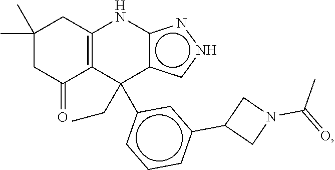 | 948779 |
| 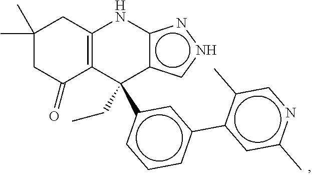 | 943515 |
| 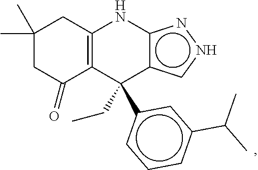 | 810375 |
| 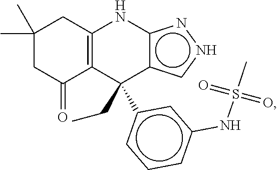 | 927341 |
| 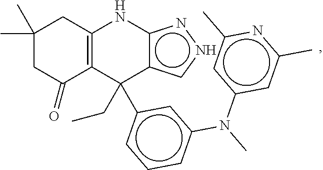 | 913256 |
| 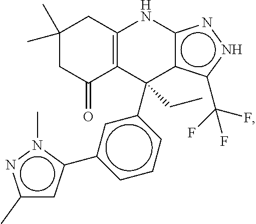 | 947383 |
| 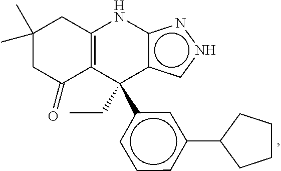 | 949399 |
| 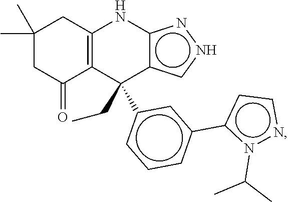 | 946092 |
| 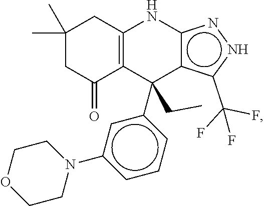 | 816886 |
| 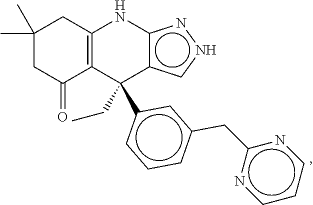 | 947499 |
| 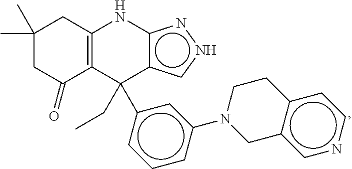 | 917148 |
| 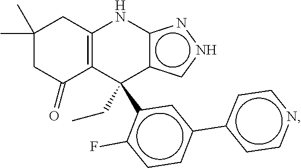 | 893324 |
| 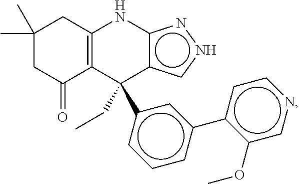 | 945471 |
| 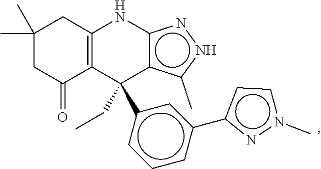 | 913039 |
| 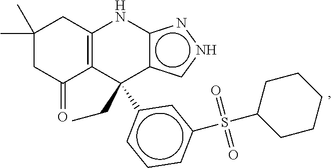 | 943547 |
| 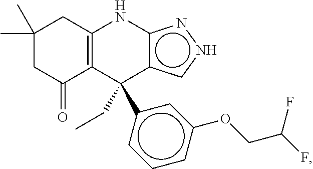 | 949113 |
| 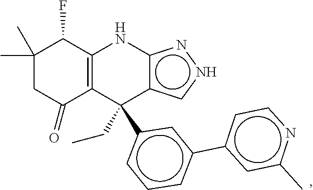 | 946102 |
| 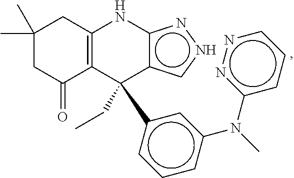 | 911976 |
| 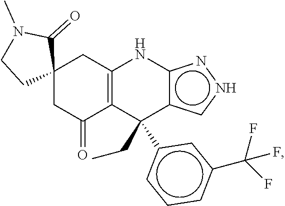 | 950330 |
| 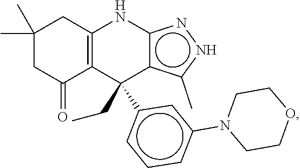 | 889582 |
| 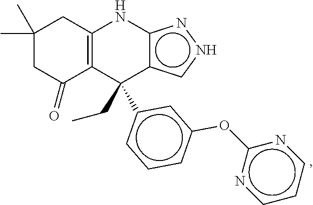 | 947818 |
| 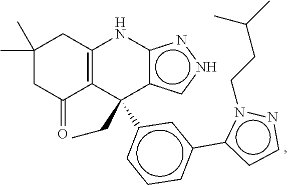 | 946096 |
| 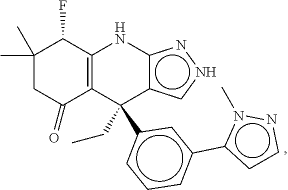 | 957509 |
| 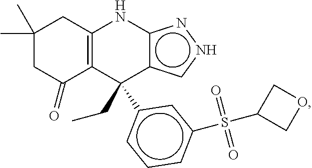 | 946003 |
| 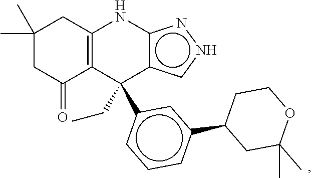 | 912090 |
| 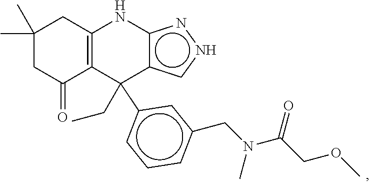 | 865196 |
| 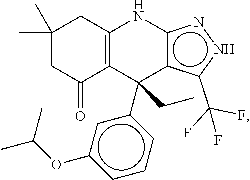 | 947872 |
| 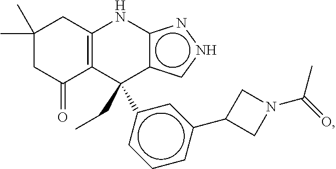 | 949131 |
| 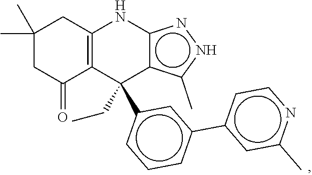 | 913051 |
| 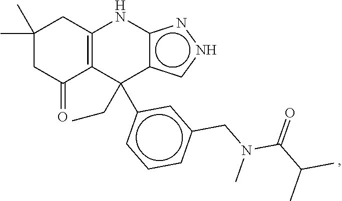 | 865195 |
| 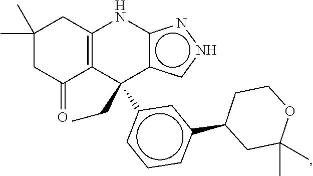 | 912088 |
| 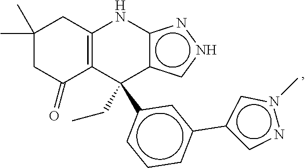 | 819576 |
| 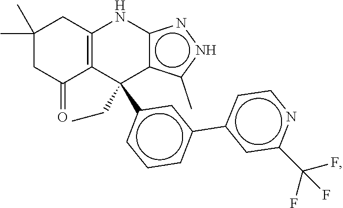 | 913049 |
| 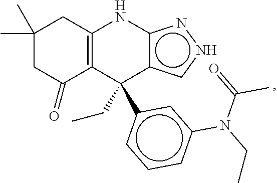 | 909215 |
| 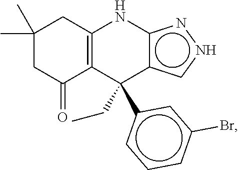 | 790734 |
| 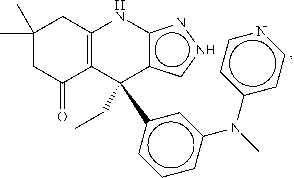 | 874261 |
| 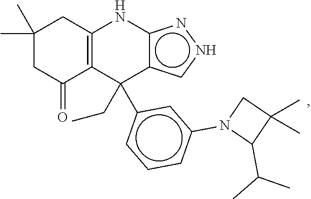 | 902969 |
| 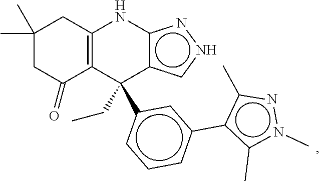 | 911991 |
| 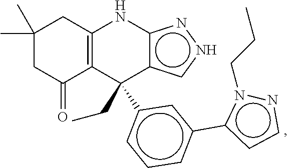 | 946094 |
| 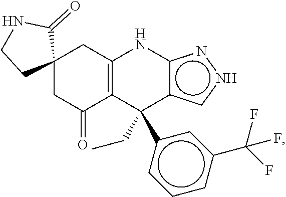 | 950331 |
| 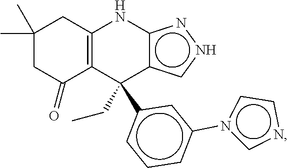 | 874533 |
| 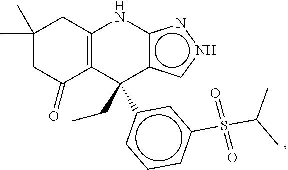 | 913134 |
| 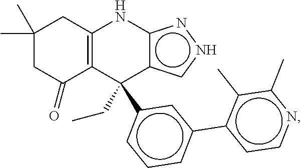 | 943517 |
| 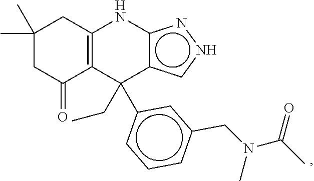 | 865194 |
| 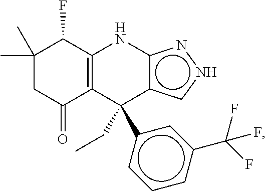 | 837753 |
| 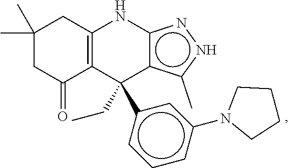 | 883875 |
| 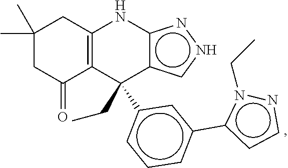 | 946090 |
| 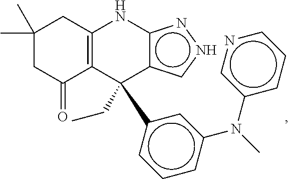 | 874299 |
| 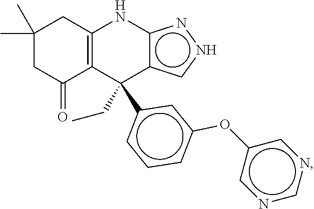 | 945939 |
| 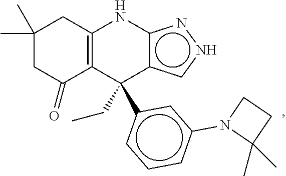 | 913141 |
| 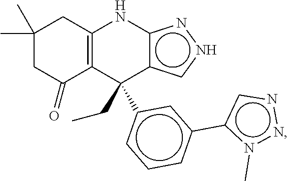 | 947824 |
| 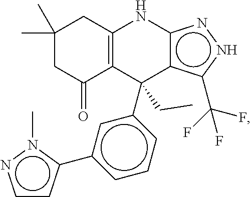 | 947365 |
| 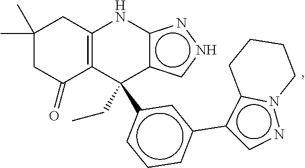 | 945544 |
| 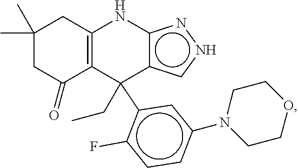 | 909203 |
| 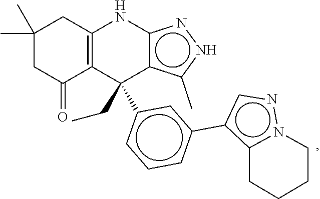 | 913033 |
| 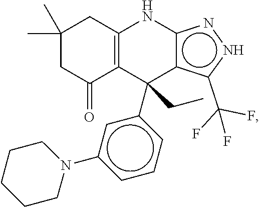 | 816799 |
| 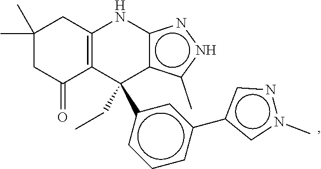 | 913038 |
| 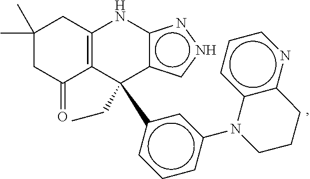 | 917079 |
| 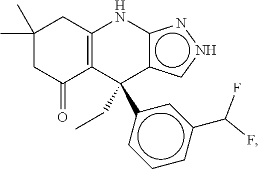 | 826716 |
| 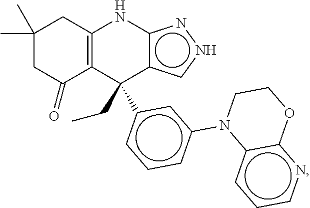 | 932643 |
| 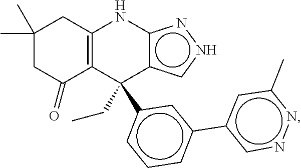 | 943995 |
| 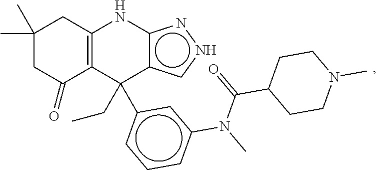 | 912167 |
| 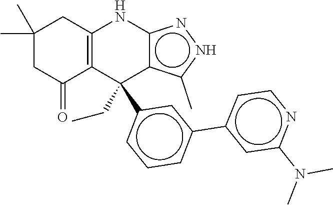 | 913048 |
| 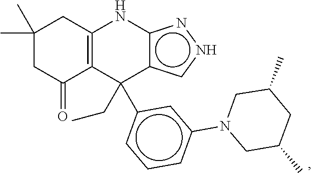 | 885237 |
| 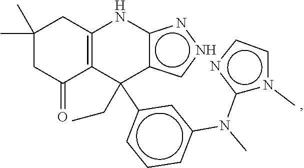 | 885402 |
| 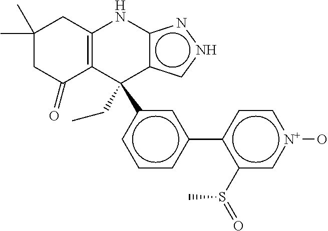 | 947571 |
| 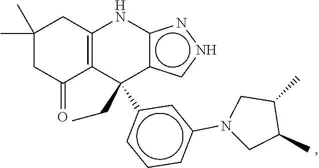 | 924312 |
| 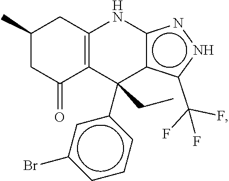 | 828850 |
| 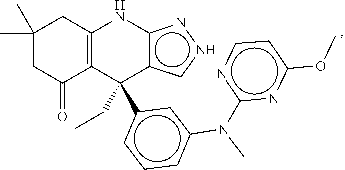 | 947337 |
| 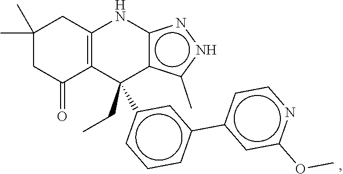 | 913016 |
| 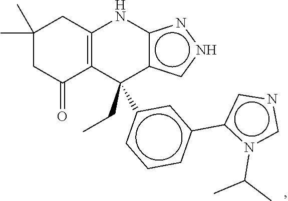 | 945729 |
| 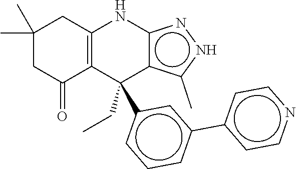 | 913054 |
| 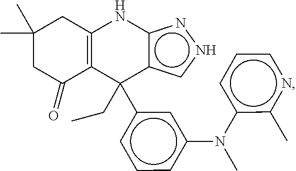 | 885355 |
| 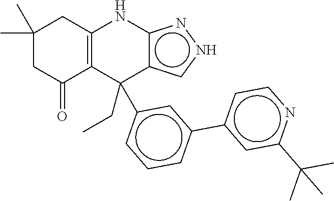 | 947490 |
| 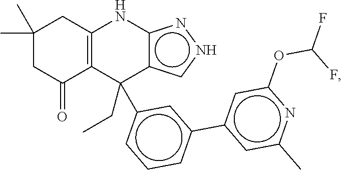 | 945463 |
| 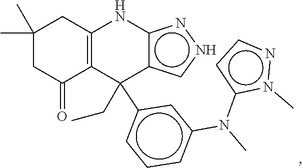 | 889604 |
| 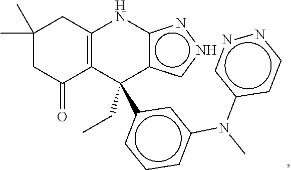 | 915630 |
| 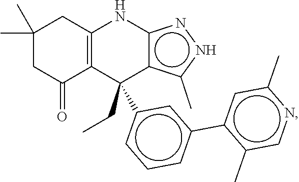 | 913019 |
| 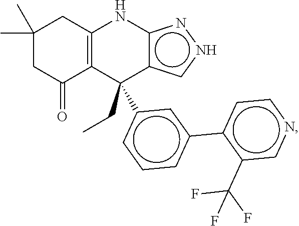 | 943918 |
| 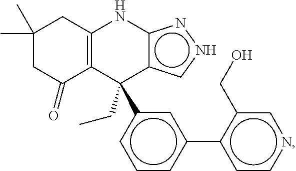 | 945479 |
| 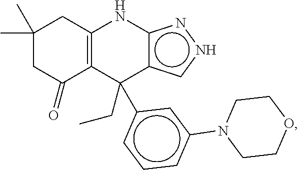 | 801318 |
| 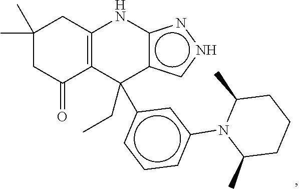 | 865172 |
| 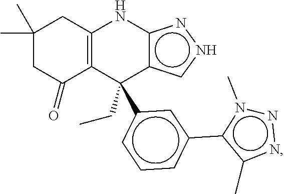 | 946100 |
| 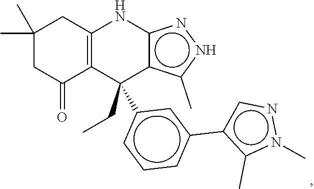 | 913024 |
| 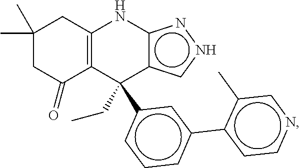 | 943991 |
| 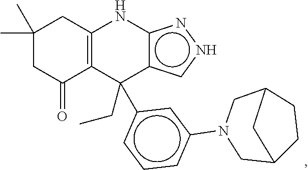 | 885099 |
| 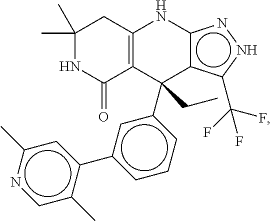 | 947364 |
| 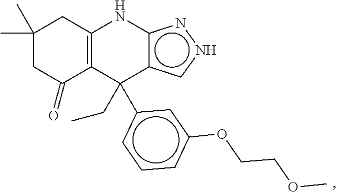 | 801446 |
| 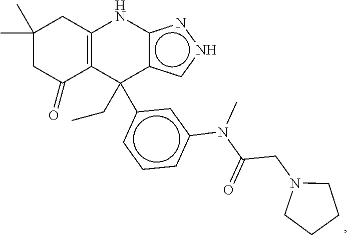 | 909202 |
| 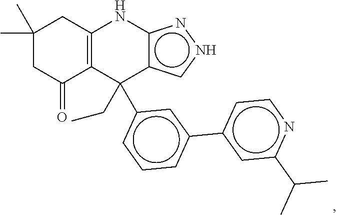 | 930569 |
| 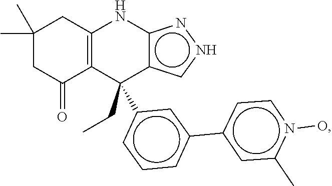 | 946055 |
| 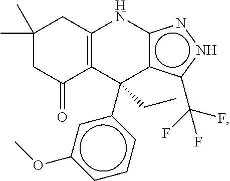 | 884118 |
| 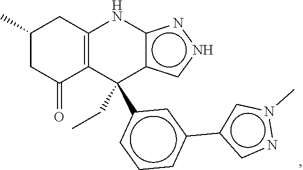 | 823724 |
| 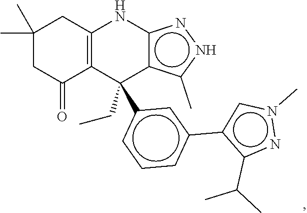 | 913040 |
| 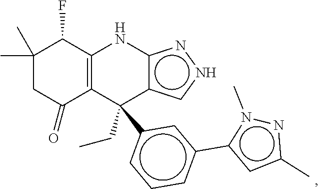 | 947657 |
| 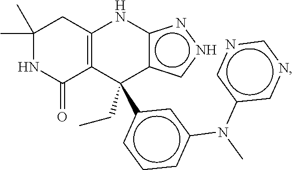 | 915401 |
| 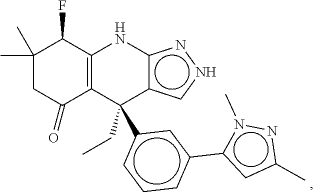 | 947656 |
| 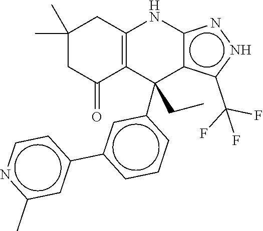 | 917289 |
| 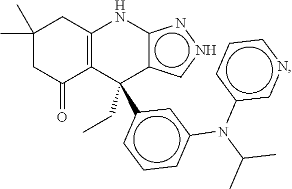 | 917087 |
| 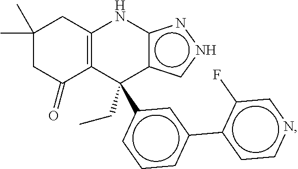 | 943519 |
| 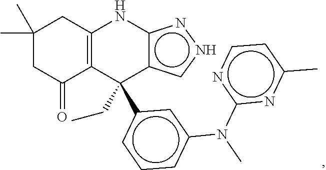 | 945329 |
| 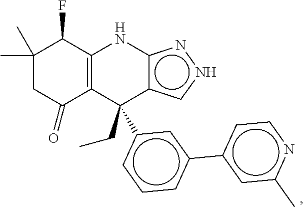 | 946103 |
| 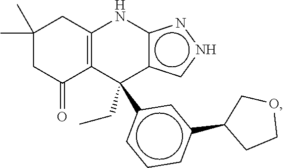 | 889565 |
| 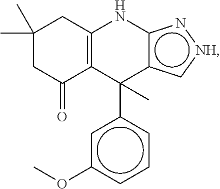 | 792342 |
| 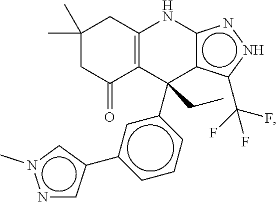 | 811567 |
| 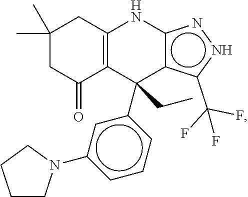 | 819575 |
| 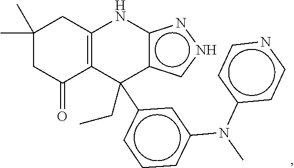 | 853910 |
| 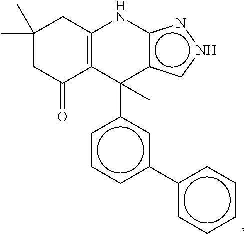 | 789513 |
| 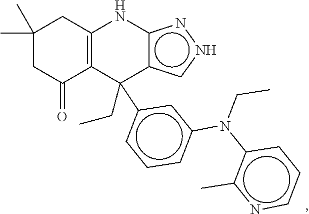 | 885367 |
| 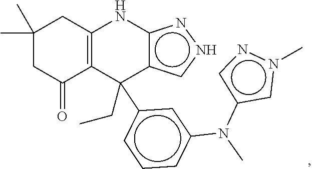 | 885382 |
| 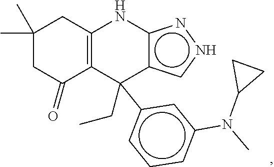 | 945466 |
| 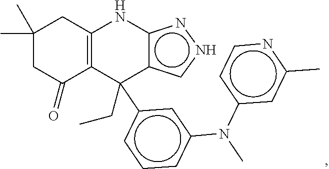 | 917149 |
| 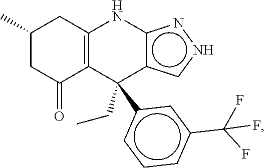 | 810452 |
| 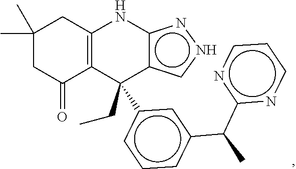 | 948057 |
| 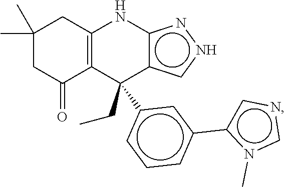 | 945620 |
| 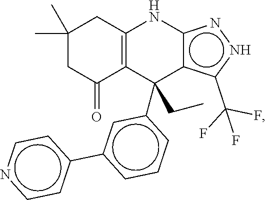 | 917292 |
| 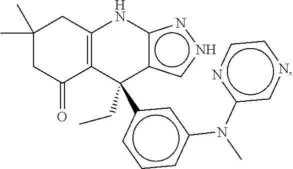 | 915340 |
| 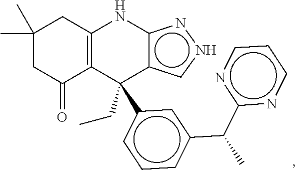 | 948061 |
| 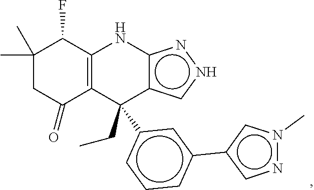 | 957504 |
| 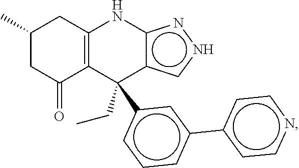 | 874316 |
| 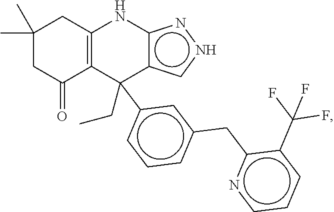 | 947548 |
| 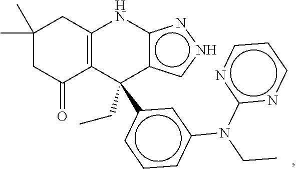 | 947334 |
| 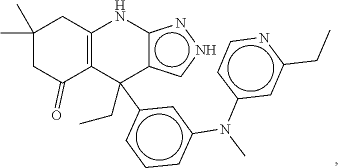 | 945600 |
| 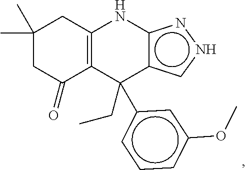 | 801447 |
| 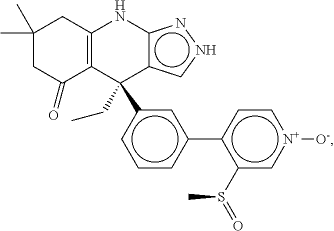 | 947570 |
| 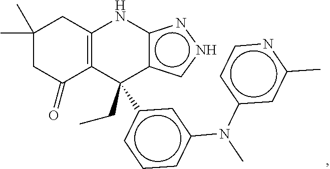 | 932645 |
| 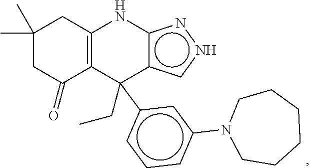 | 885088 |
| 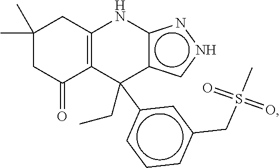 | 945601 |
| 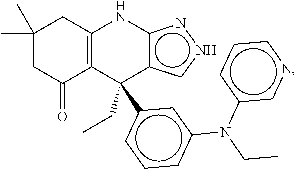 | 917089 |
| 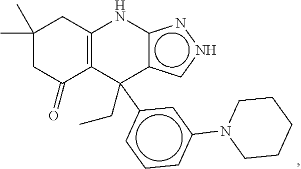 | 801320 |
| 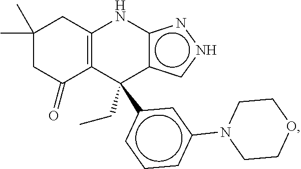 | 810363 |
| 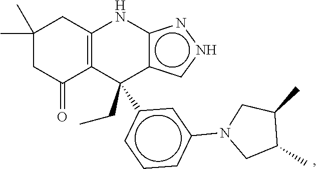 | 924504 |
| 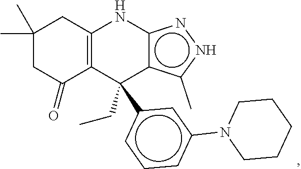 | 884115 |
| 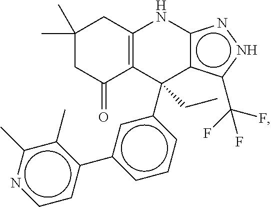 | 947328 |
| 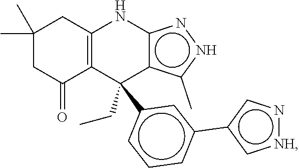 | 913044 |
| 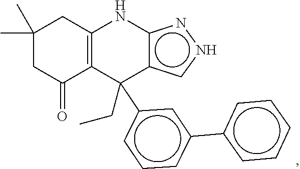 | 789514 |
| 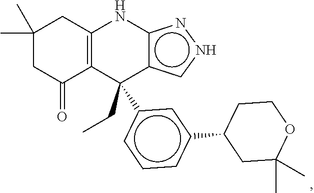 | 912089 |
| 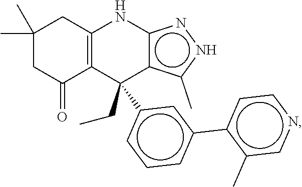 | 913022 |
| 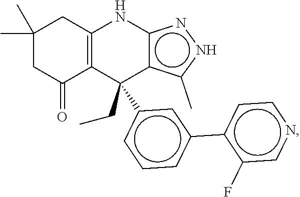 | 913017 |
| 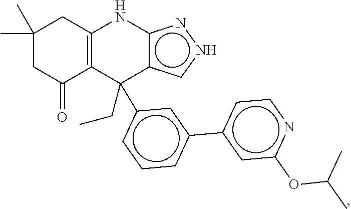 | 932626 |
| 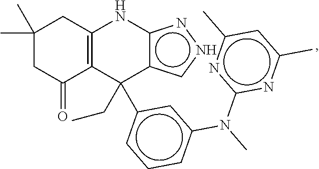 | 926304 |
| 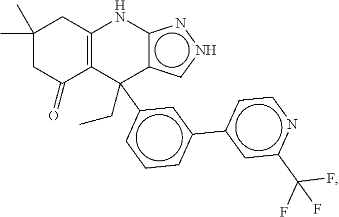 | 913350 |
| 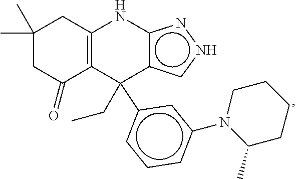 | 885217 |
| 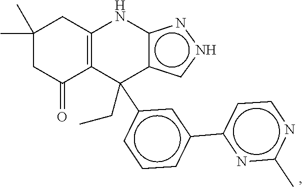 | 948088 |
| 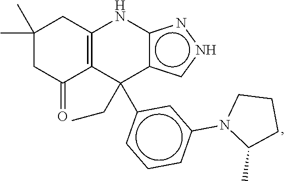 | 885188 |
| 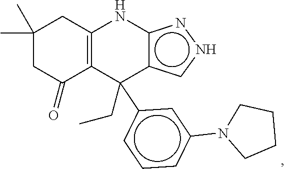 | 801317 |
| 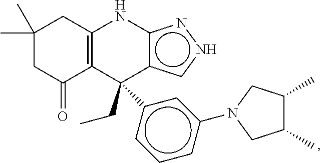 | 918326 |
| 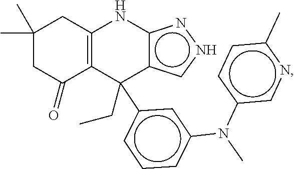 | 918272 |
| 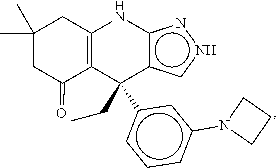 | 889631 |
| 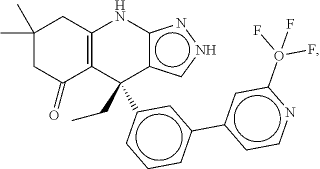 | 943988 |
| 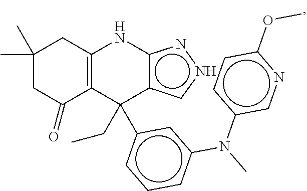 | 913252 |
| 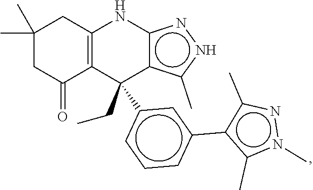 | 913026 |
| 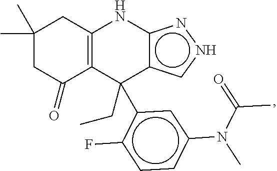 | 913124 |
| 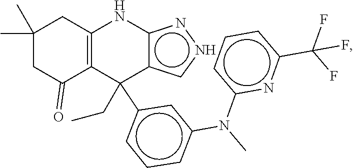 | 913122 |
| 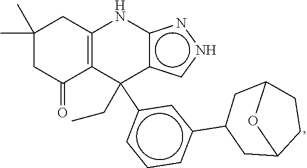 | 889622 |
| 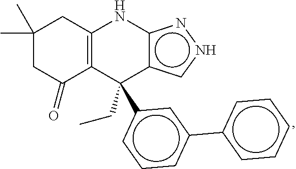 | 801368 |
| 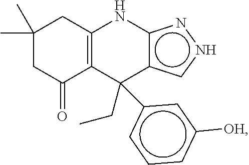 | 930334 |
| 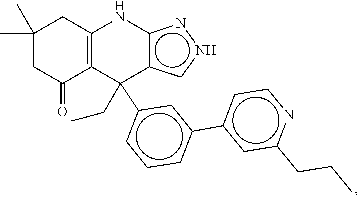 | 945467 |
| 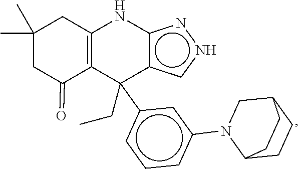 | 885120 |
| 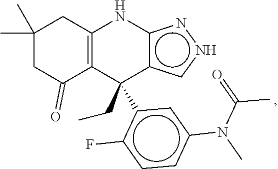 | 929450 |
| 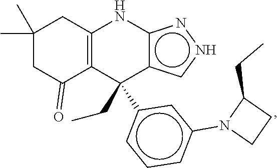 | 911969 |
| 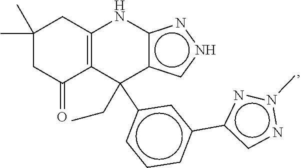 | 947492 |
| 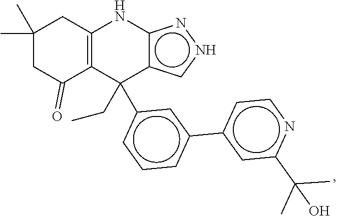 | 943536 |
| 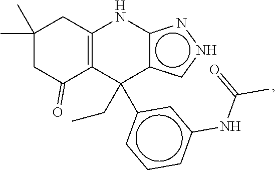 | 877310 |
| 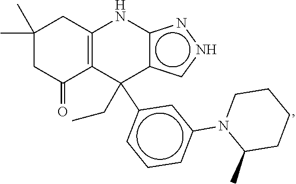 | 885228 |
| 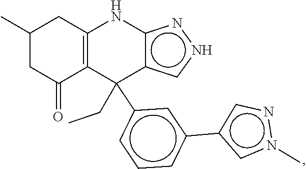 | 816752 |
| 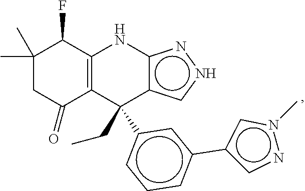 | 957505 |
| 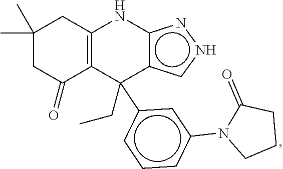 | 865171 |
| 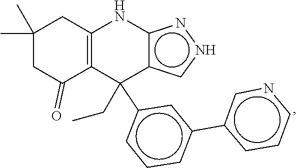 | 791272 |
| 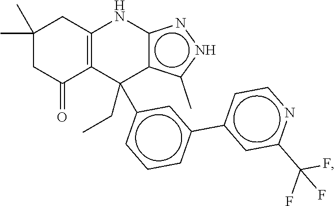 | 883989 |
| 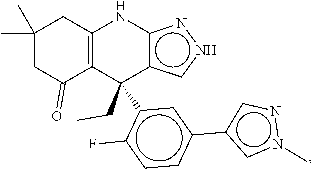 | 909220 |
| 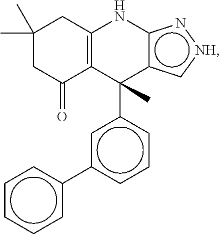 | 801367 |
| 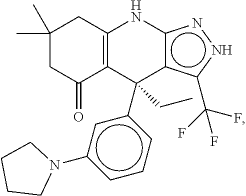 | 819574 |
| 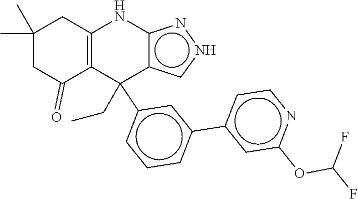 | 932629 |
| 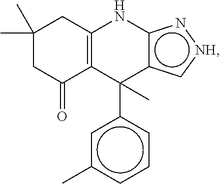 | 785736 |
| 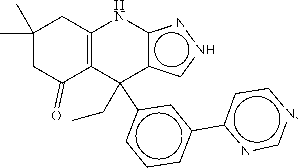 | 806637 |
| 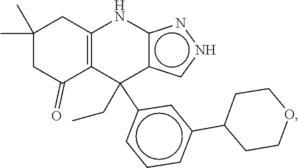 | 791273 |
| 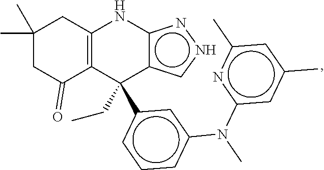 | 945610 |
| 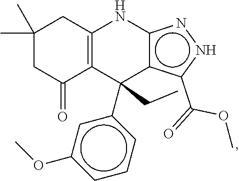 | 901480 |
| 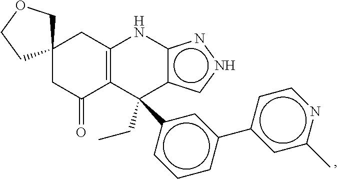 | 947407 |
| 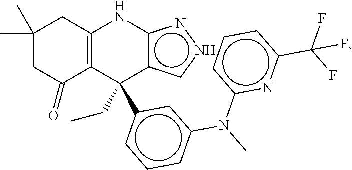 | 927338 |
| 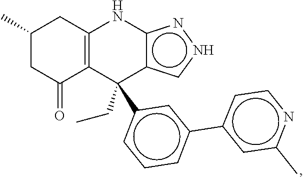 | 932657 |
| 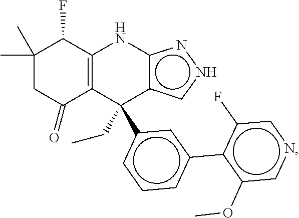 | 948548 |
| 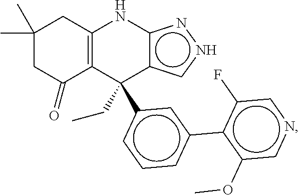 | 947512 |
| 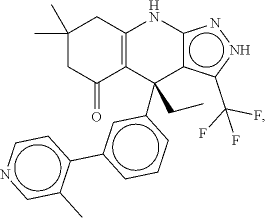 | 947325 |
| 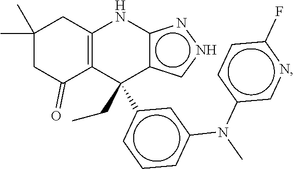 | 932647 |
| 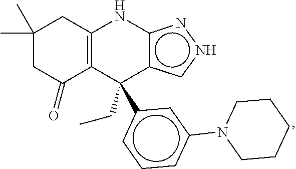 | 810369 |
| 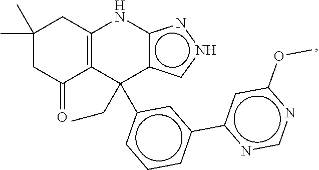 | 948093 |
| 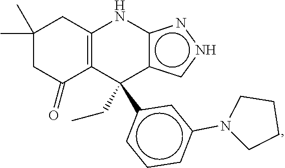 | 810510 |
| 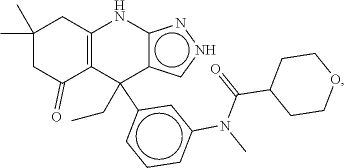 | 912070 |
| 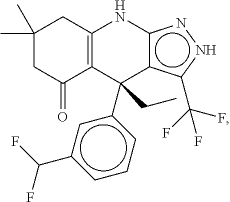 | 945708 |
| 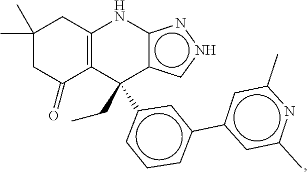 | 913222 |
| 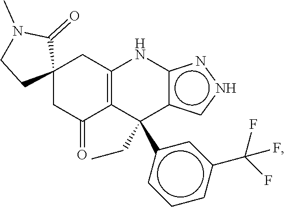 | 950326 |
| 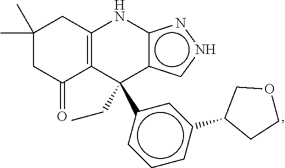 | 889558 |
| 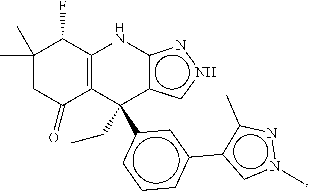 | 947653 |
| 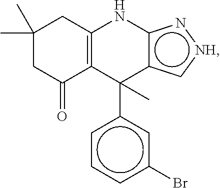 | 789277 |
| 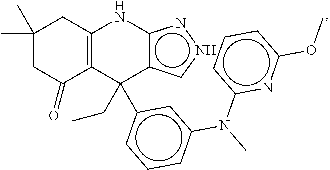 | 913121 |
| 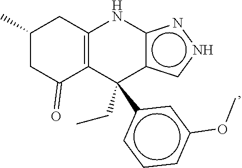 | 811559 |
| 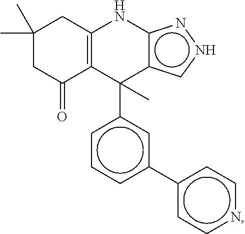 | 790730 |
| 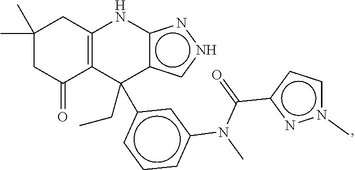 | 917130 |
| 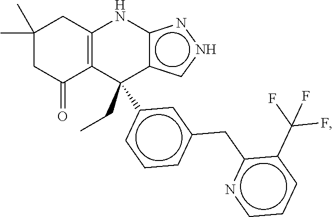 | 947664 |
| 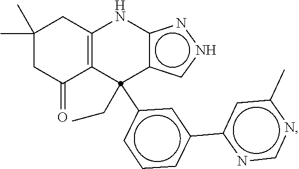 | 948089 |
| 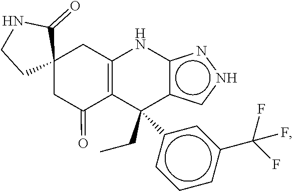 | 950333 |
| 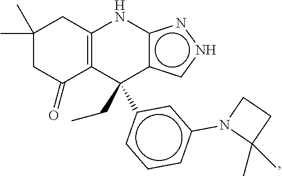 | 913140 |
| 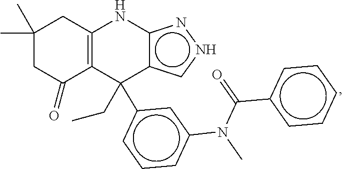 | 909201 |
| 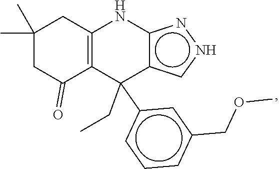 | 801323 |
| 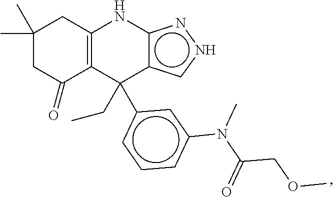 | 889580 |
| 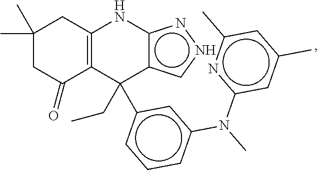 | 943538 |
| 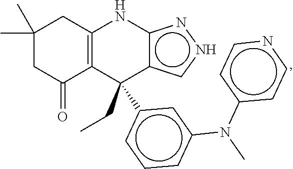 | 874260 |
| 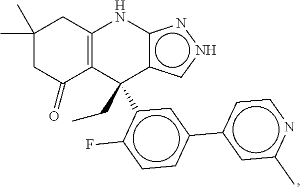 | 909212 |
| 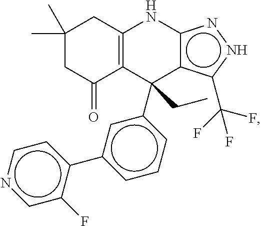 | 947327 |
| 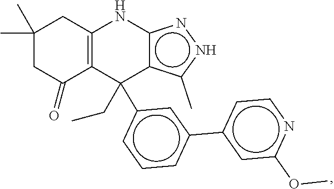 | 883985 |
| 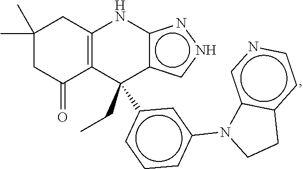 | 912003 |
| 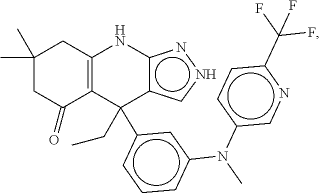 | 932634 |
| 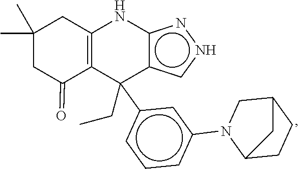 | 885110 |
| 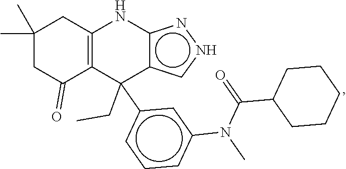 | 912065 |
| 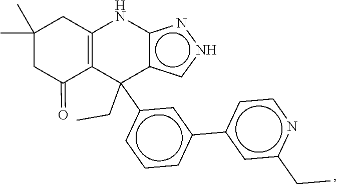 | 947493 |
| 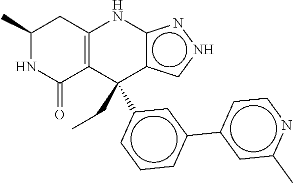 | 932658 |
| 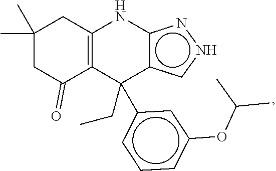 | 801448 |
| 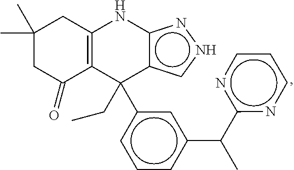 | 947434 |
| 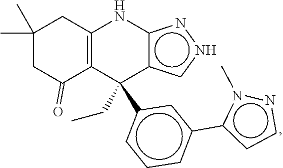 | 911993 |
| 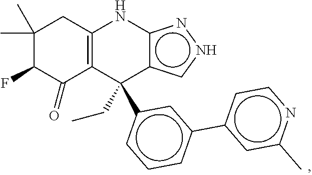 | 945475 |
| 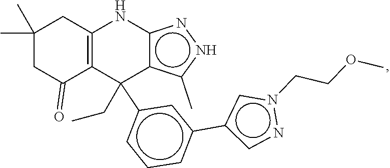 | 883996 |
| 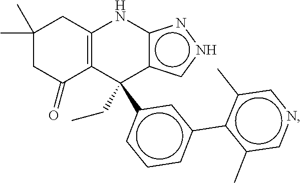 | 947659 |
| 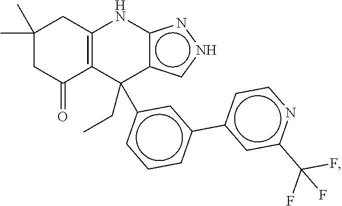 | 913348 |
| 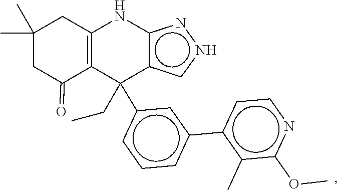 | 932628 |
| 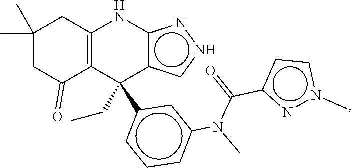 | 929442 |
| 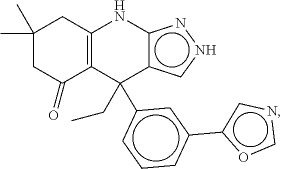 | 944025 |
| 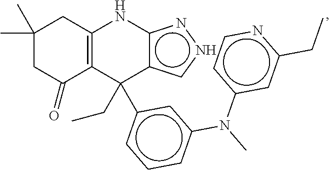 | 917147 |
| 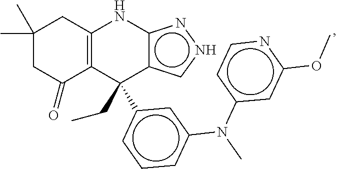 | 932642 |
| 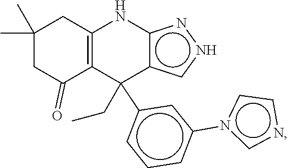 | 854926 |
| 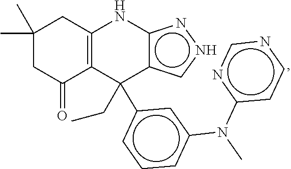 | 902970 |
| 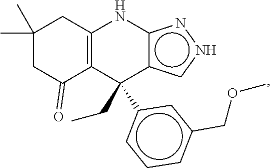 | 810360 |
| 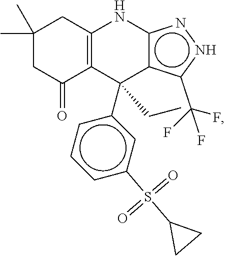 | 945715 |
| 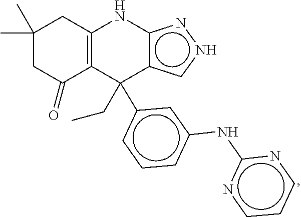 | 913334 |
| 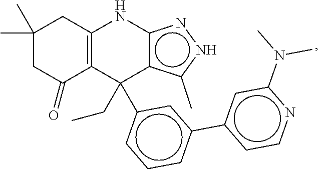 | 883990 |
| 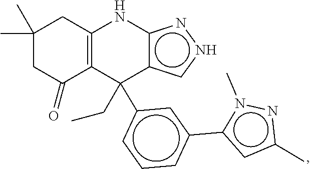 | 929402 |
| 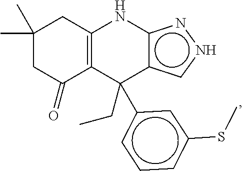 | 810508 |
| 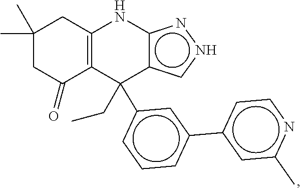 | 840857 |
| 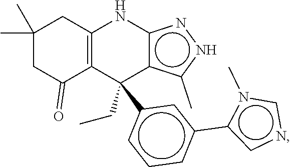 | 913037 |
| 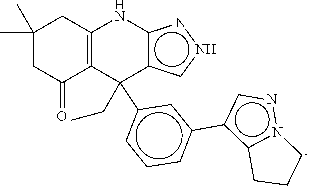 | 947494 |
| 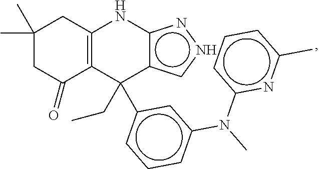 | 913255 |
| 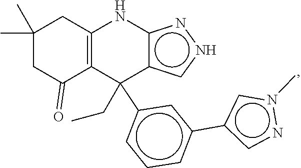 | 816871 |
| 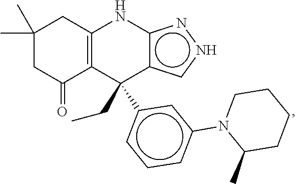 | 917209 |
| 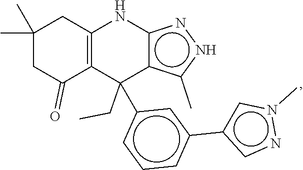 | 884000 |
| 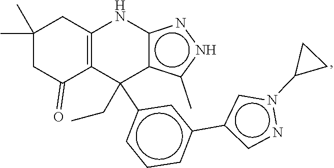 | 883997 |
| 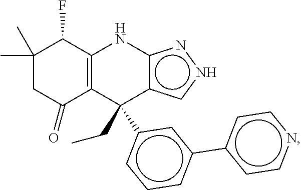 | 959341 |
| 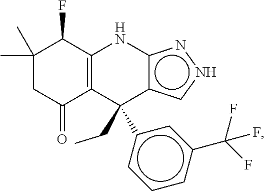 | 837683 |
| 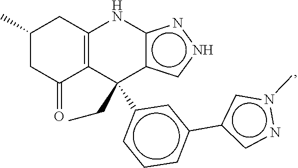 | 823720 |
| 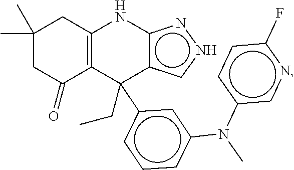 | 917152 |
| 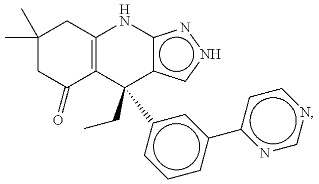 | 810488 |
| 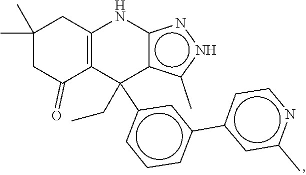 | 883987 |
| 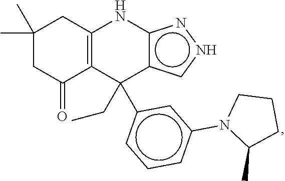 | 885204 |
| 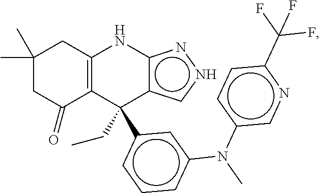 | 945474 |
| 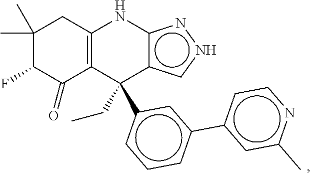 | 945476 |
| 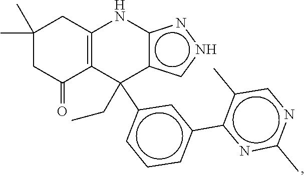 | 948091 |
| 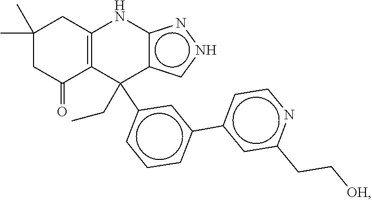 | 945992 |
| 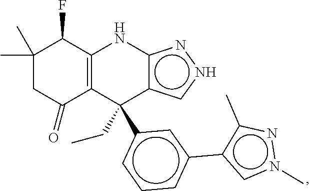 | 947652 |
| 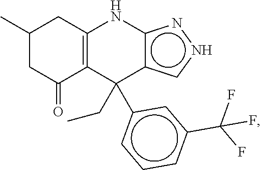 | 801397 |
| 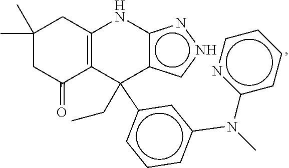 | 874294 |
| 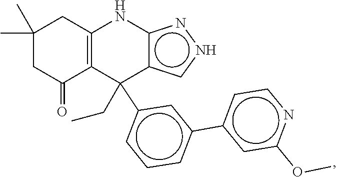 | 883850 |
| 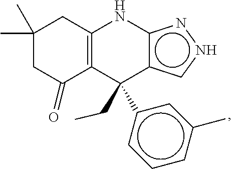 | 789560 |
| 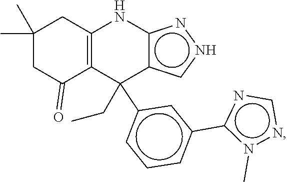 | 943532 |
| 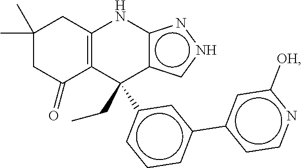 | 943992 |
| 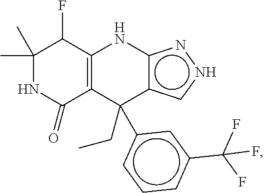 | 819622 |
| 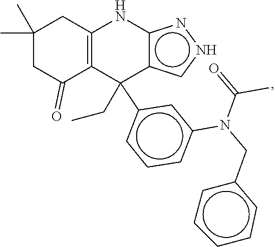 | 884110 |
| 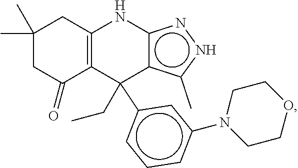 | 874312 |
| 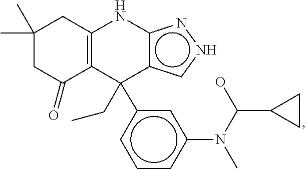 | 912067 |
| 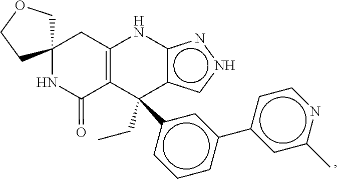 | 947406 |
| 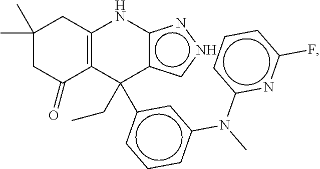 | 918274 |
| 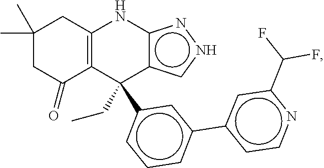 | 943919 |
| 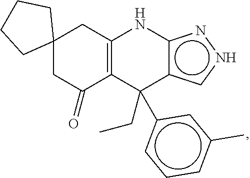 | 789565 |
| 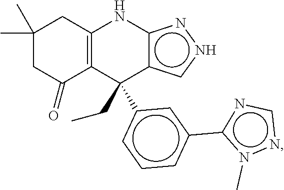 | 945472 |
| 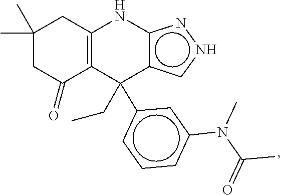 | 801319 |
| 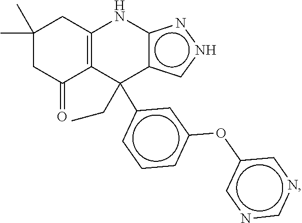 | 917212 |
| 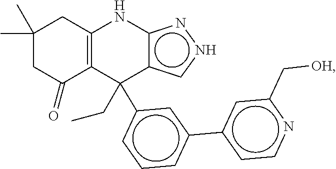 | 943540 |
| 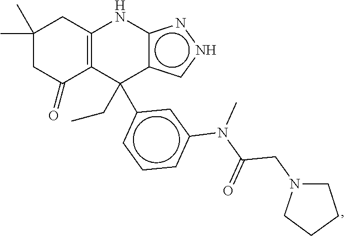 | 913125 |
| 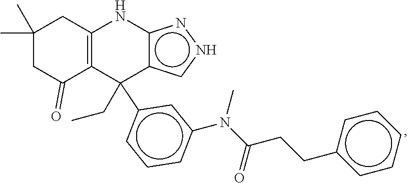 | 912066 |
| 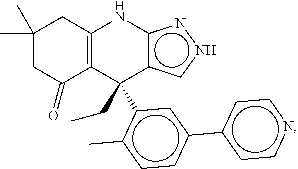 | 893323 |
| 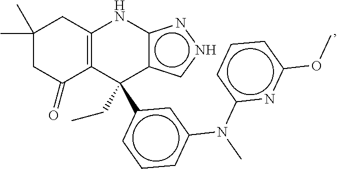 | 932666 |
| 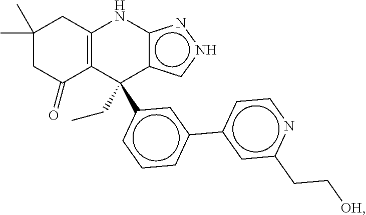 | 947810 |
| 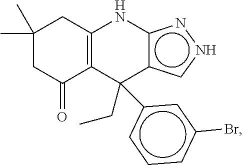 | 789312 |
| 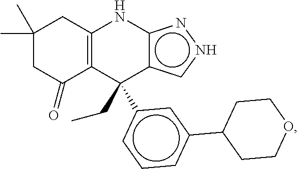 | 819638 |
| 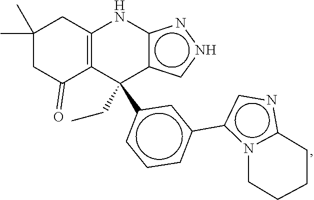 | 945607 |
| 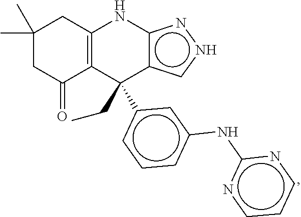 | 945943 |
| 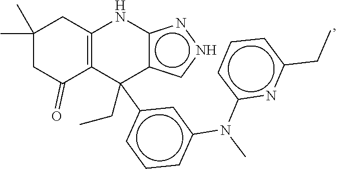 | 944023 |
| 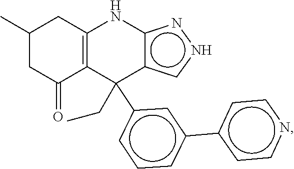 | 856947 |
| 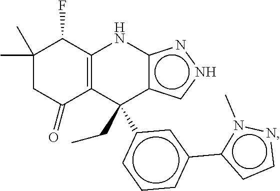 | 957506 |
| 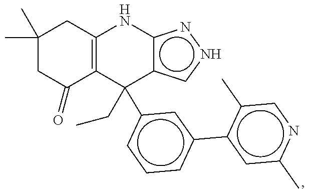 | 913346 |
| 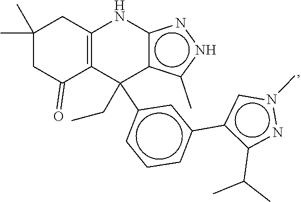 | 883998 |
| 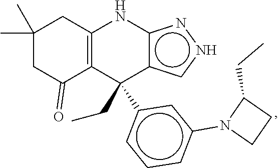 | 911970 |
| 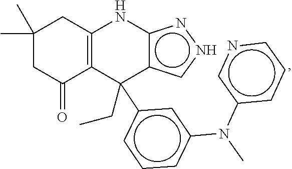 | 865168 |
| 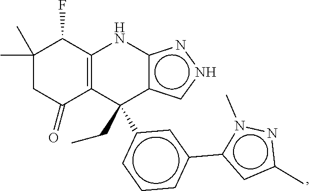 | 947655 |
| 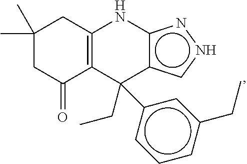 | 949083 |
| 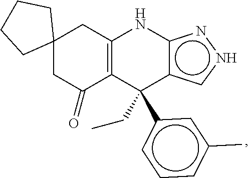 | 801312 |
| 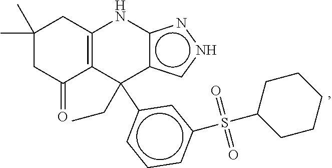 | 918273 |
| 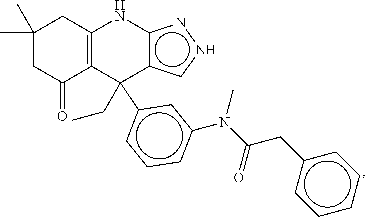 | 912071 |
| 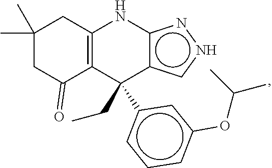 | 811564 |
| 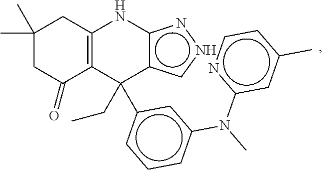 | 943537 |
| 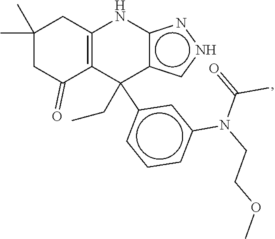 | 893306 |
| 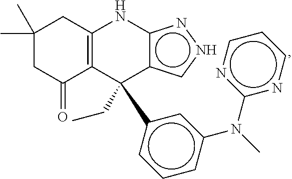 | 911981 |
| 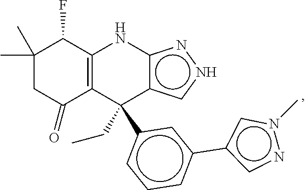 | 957511 |
| 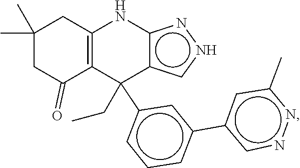 | 926844 |
| 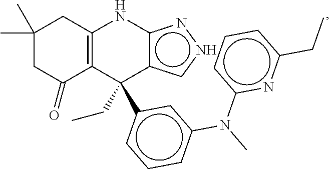 | 945730 |
| 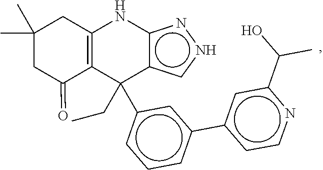 | 944029 |
| 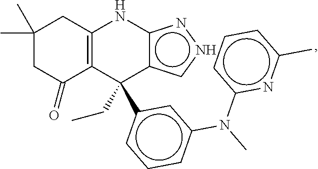 | 932664 |
| 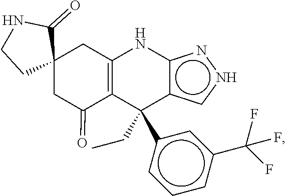 | 950324 |
| 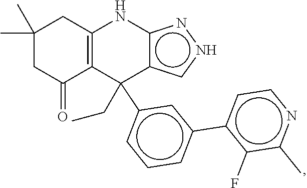 | 918281 |
| 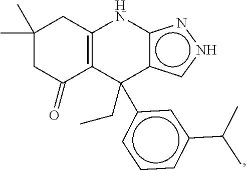 | 801322 |
| 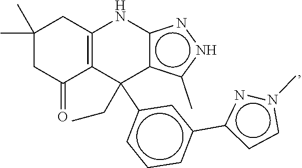 | 883999 |
| 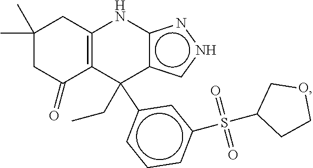 | 944018 |
| 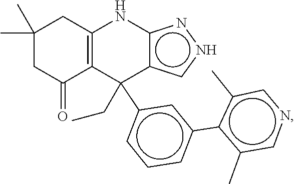 | 945703 |
| 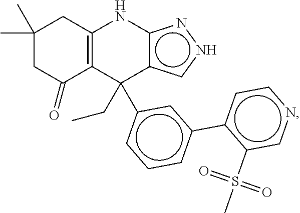 | 947569 |
| 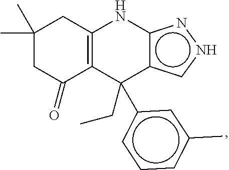 | 785738 |
| 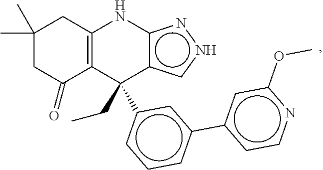 | 911988 |
| 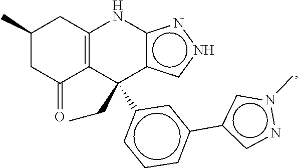 | 823721 |
| 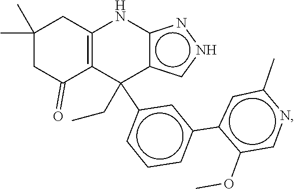 | 945468 |
| 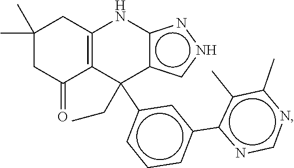 | 948090 |
| 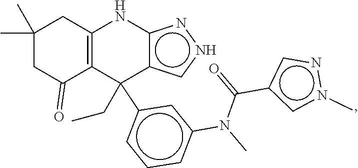 | 912068 |
| 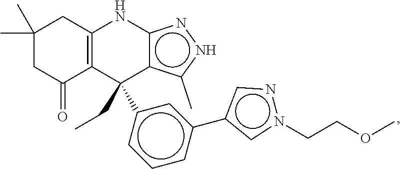 | 913027 |
| 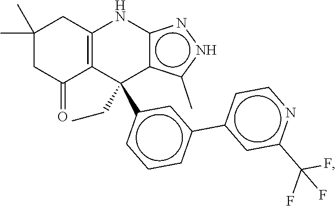 | 913020 |
| 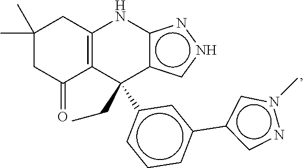 | 819577 |
| 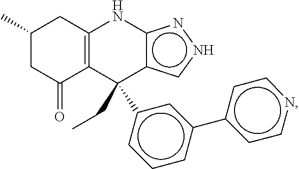 | 874318 |
| 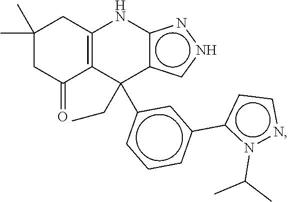 | 929399 |
| 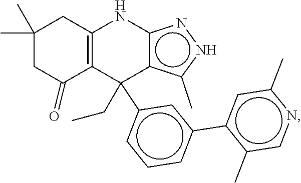 | 883988 |
| 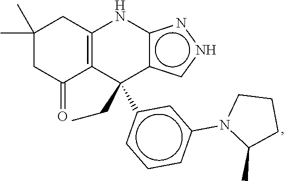 | 918321 |
| 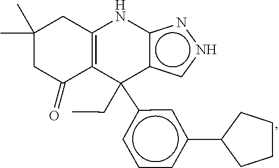 | 949082 |
| 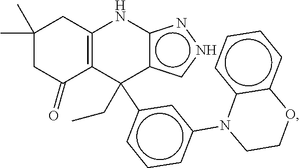 | 918280 |
| 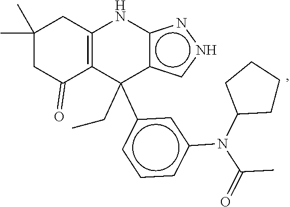 | 912069 |
| 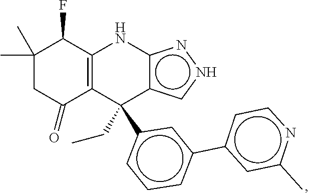 | 946104 |
| 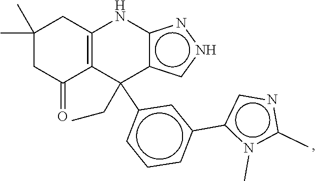 | 943531 |
| 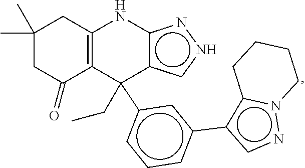 | 926836 |
| 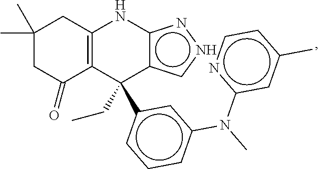 | 945609 |
| 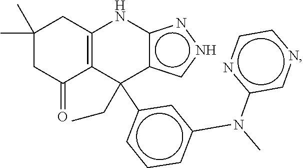 | 902971 |
| 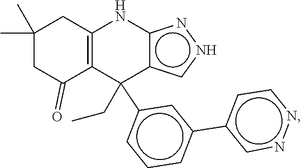 | 927342 |
| 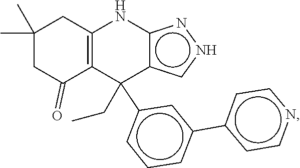 | 883984 |
| 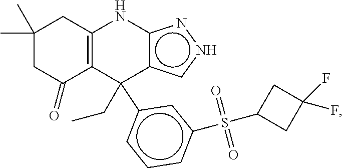 | 944019 |
| 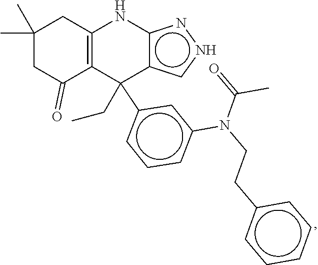 | 912064 |
| 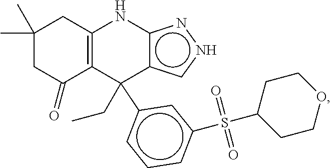 | 927344 |
| 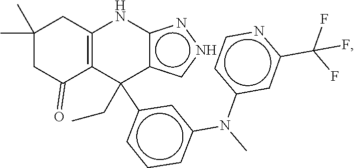 | 913254 |
| 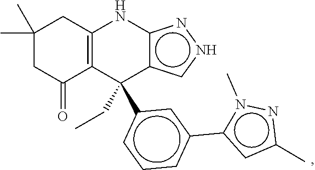 | 946097 |
| 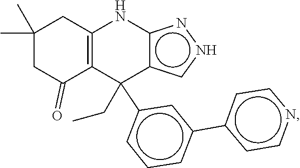 | 791271 |
| 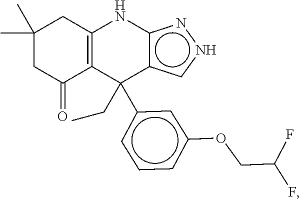 | 948793 |
| 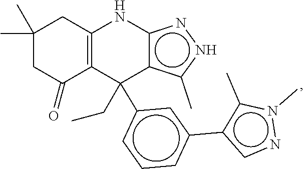 | 883993 |
| 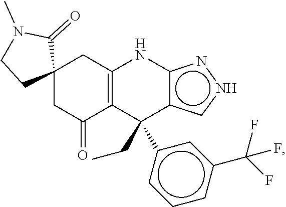 | 950329 |
| 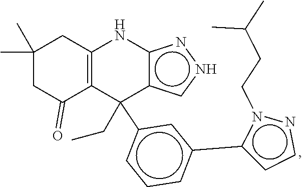 | 929401 |
| 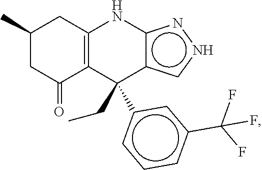 | 810451 |
| 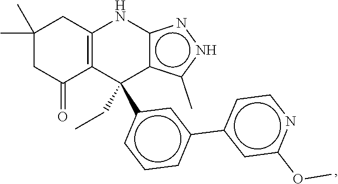 | 913053 |
| 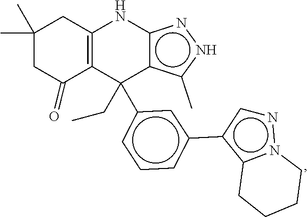 | 884002 |
| 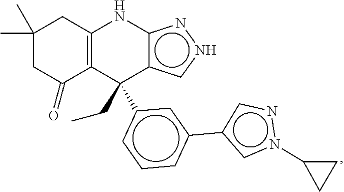 | 913028 |
| 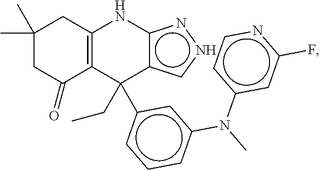 | 917146 |
| 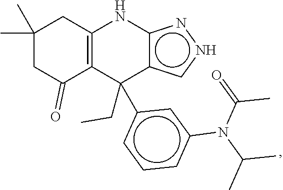 | 893305 |
| 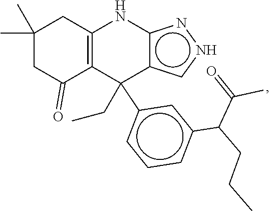 | 909205 |
| 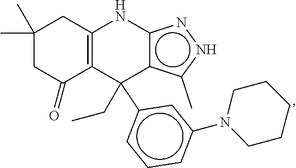 | 877311 |
| 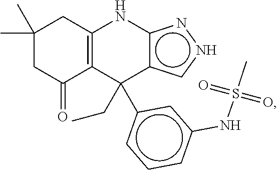 | 913253 |
| 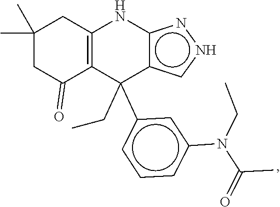 | 884109 |
| 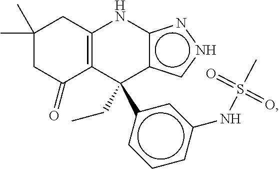 | 927348 |
| 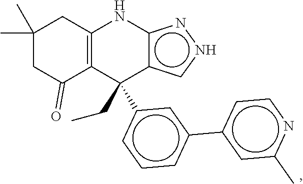 | 865169 |
| 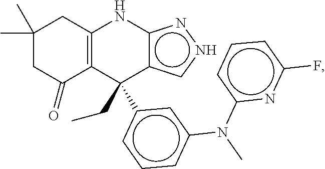 | 932636 |
| 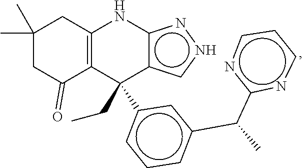 | 948058 |
| 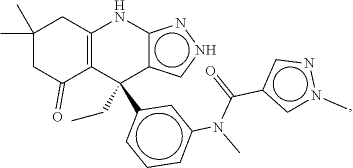 | 913310 |
| 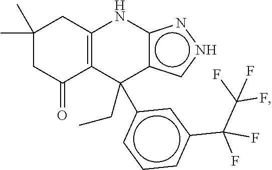 | 948615 |
| 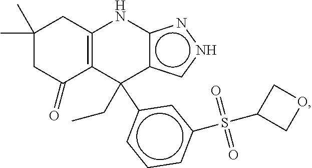 | 944017 |
| 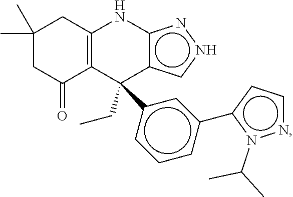 | 946091 |
| 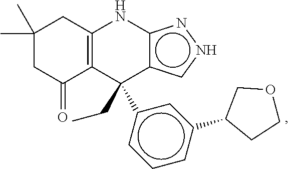 | 889556 |
| 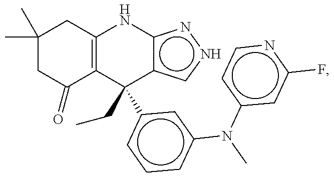 | 929444 |
| 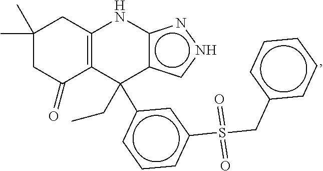 | 913258 |
| 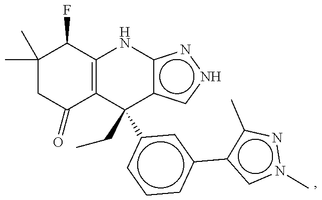 | 947650 |
| 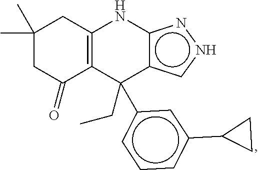 | 801316 |
| 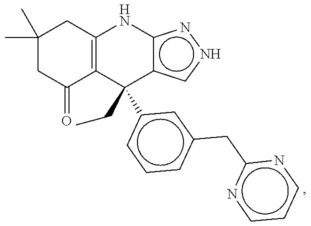 | 947498 |
| 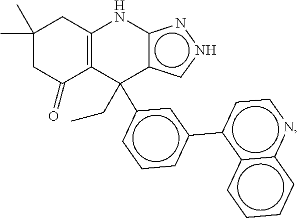 | 932631 |
| 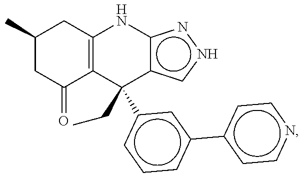 | 874317 |
| 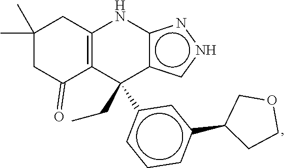 | 889557 |
| 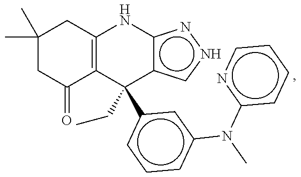 | 875568 |
| 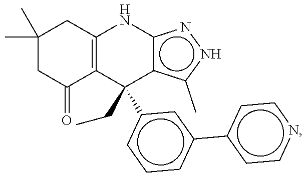 | 913015 |
| 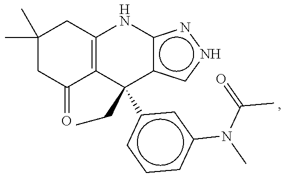 | 810358 |
| 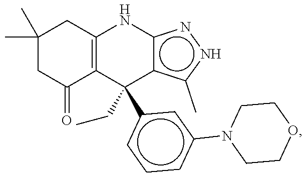 | 889581 |
| 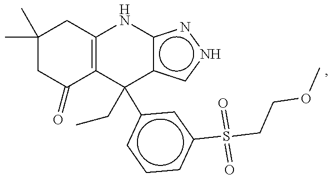 | 918271 |
| 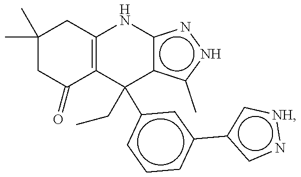 | 883994 |
| 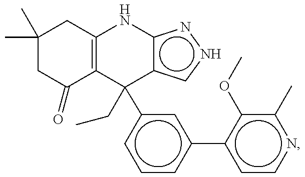 | 945458 |
| 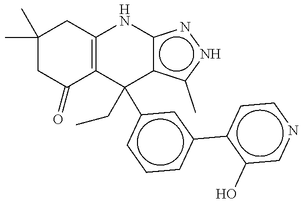 | 944024 |
| 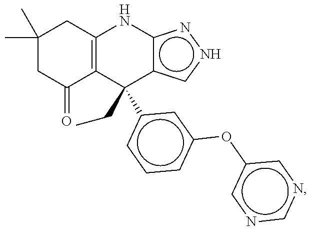 | 945933 |
| 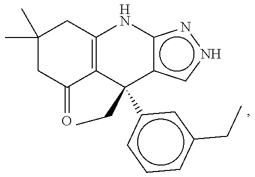 | 949401 |
| 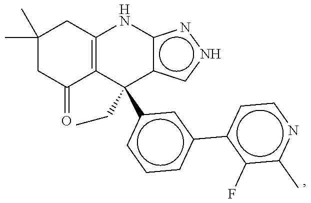 | 927347 |
| 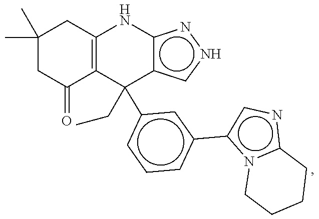 | 943535 |
| 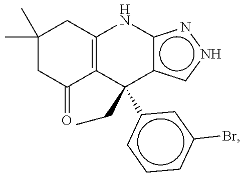 | 790733 |
| 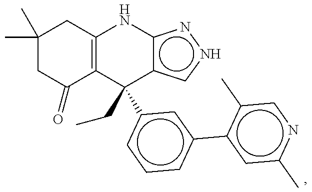 | 943514 |
| 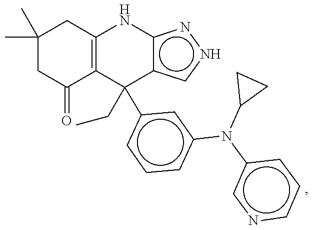 | 913274 |
| 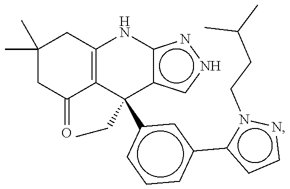 | 946095 |
| 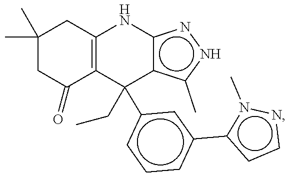 | 883992 |
| 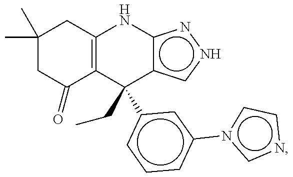 | 874531 |
| 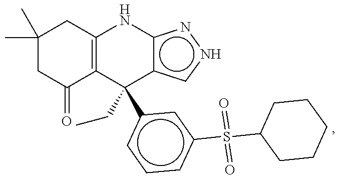 | 943541 |
| 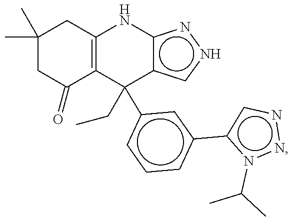 | 947397 |
| 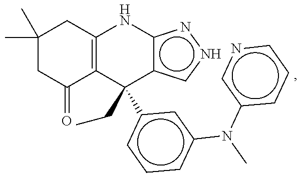 | 874298 |
| 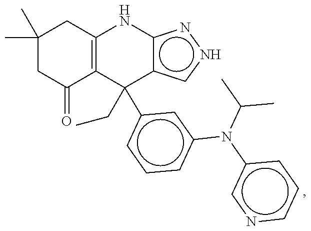 | 885344 |
| 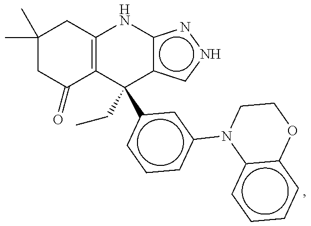 | 932655 |
| 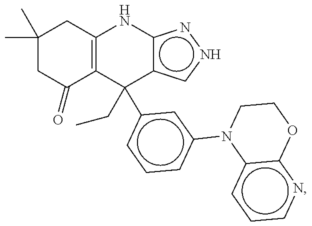 | 918279 |
| 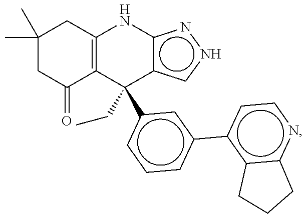 | 945606 |
| 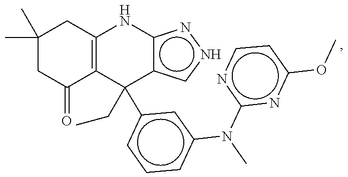 | 929387 |
| 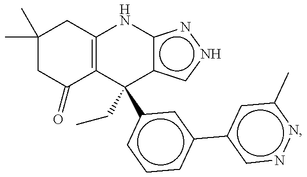 | 943994 |
| 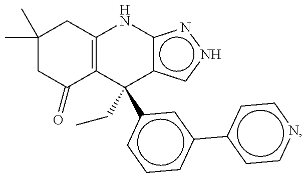 | 801413 |
| 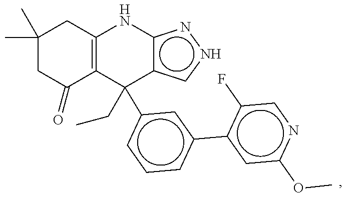 | 932630 |
| 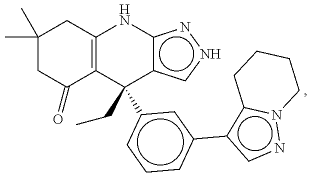 | 945543 |
| 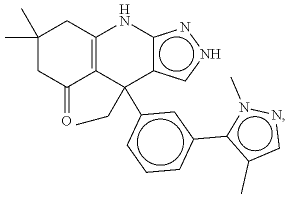 | 947491 |
| 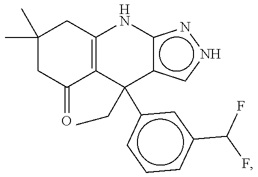 | 819564 |
| 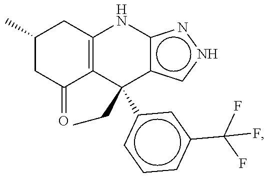 | 810450 |
| 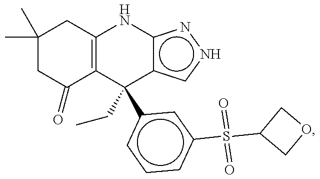 | 945998 |
| 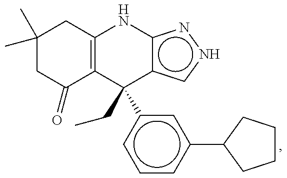 | 949397 |
| 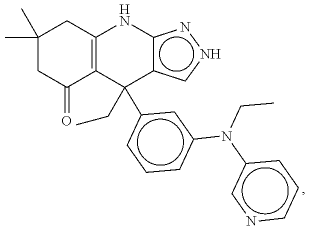 | 885331 |
| 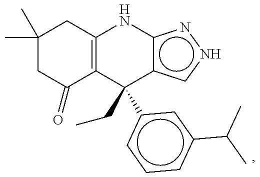 | 810373 |
| 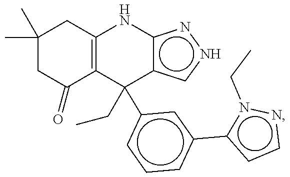 | 929398 |
| 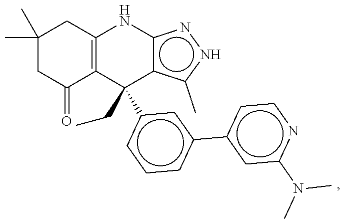 | 913021 |
| 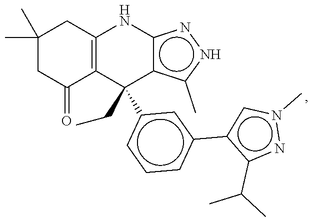 | 913029 |
| 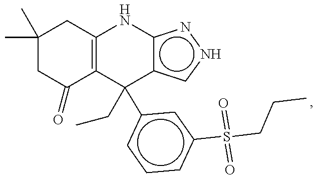 | 917131 |
| 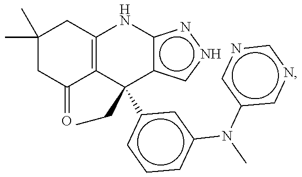 | 915400 |
| 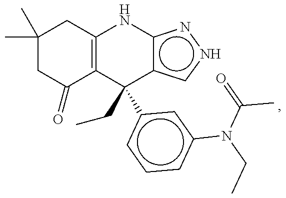 | 909214 |
| 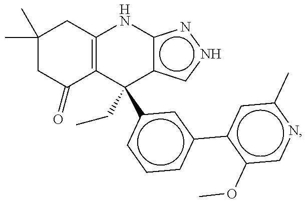 | 945995 |
| 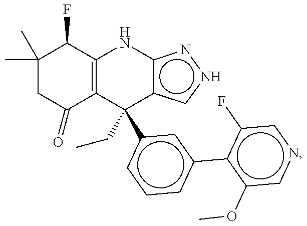 | 948547 |
| 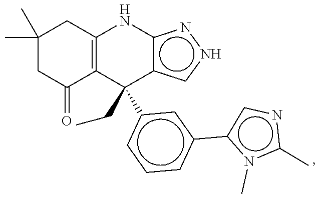 | 945613 |
| 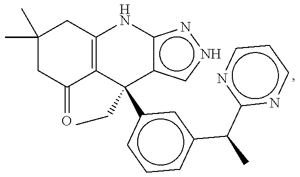 | 948060 |
| 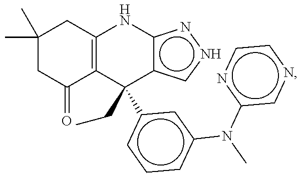 | 915316 |
| 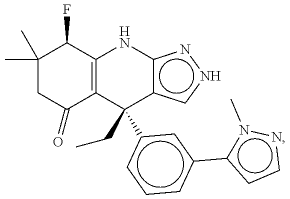 | 957507 |
| 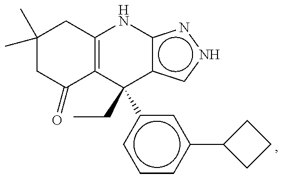 | 949521 |
| 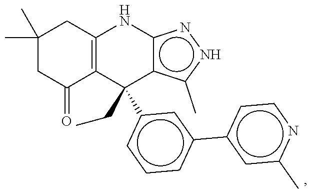 | 913018 |
| 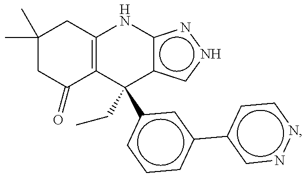 | 929454 |
| 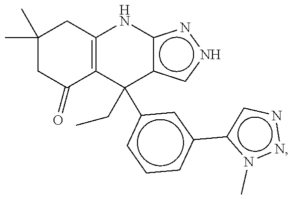 | 947398 |
| 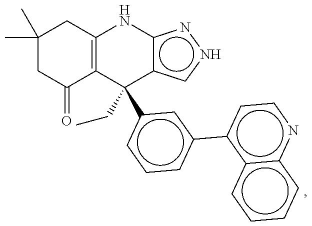 | 944031 |
| 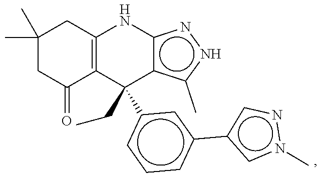 | 913031 |
| 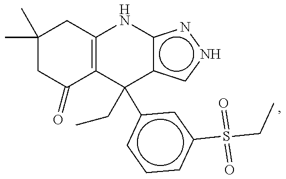 | 889579 |
| 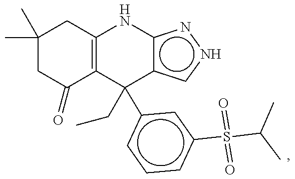 | 912169 |
| 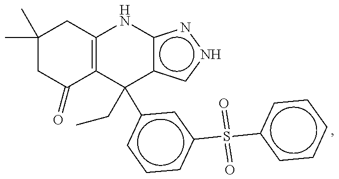 | 913123 |
| 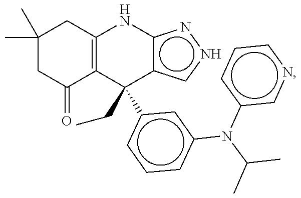 | 917086 |
| 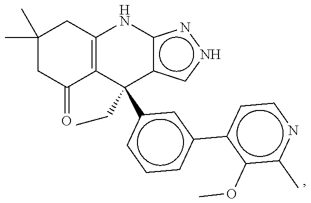 | 945997 |
| 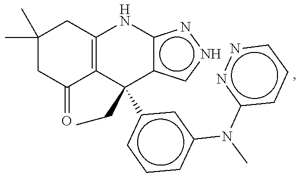 | 911975 |
| 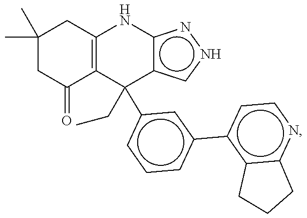 | 943534 |
| 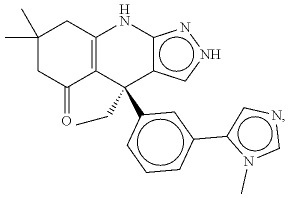 | 945605 |
| 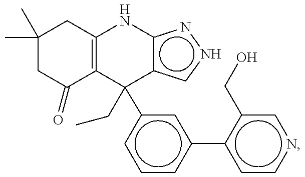 | 943539 |
| 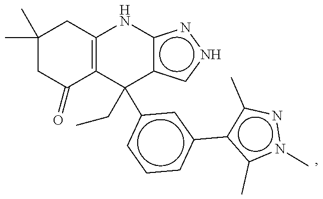 | 883851 |
| 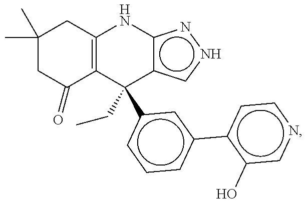 | 945724 |
| 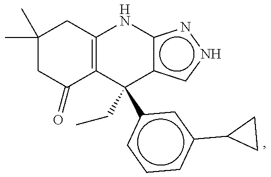 | 811557 |
| 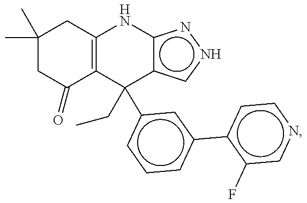 | 883986 |
| 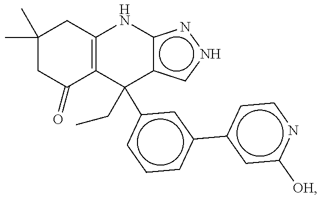 | 913352 |
| 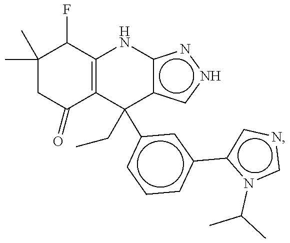 | 963155 |
| 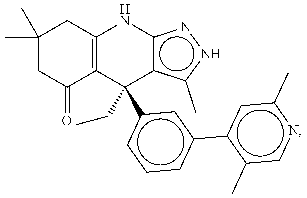 | 913050 |
| 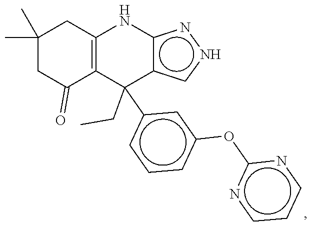 | 947400 |
| 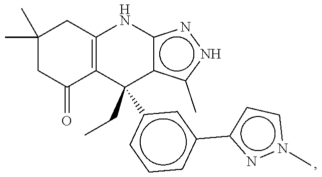 | 913030 |
| 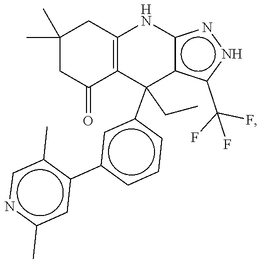 | 945530 |
| 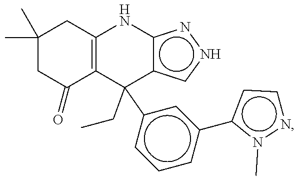 | 883852 |
| 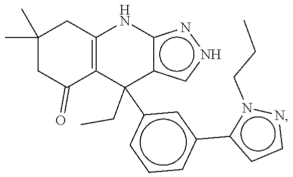 | 929400 |
| 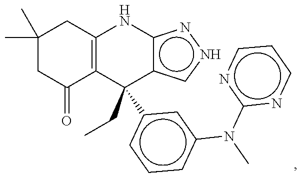 | 911980 |
| 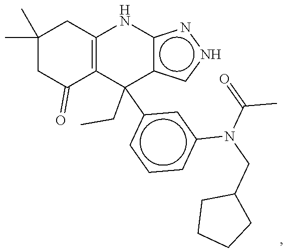 | 912166 |
| 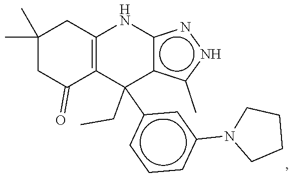 | 874314 |
| 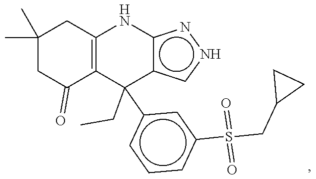 | 913257 |
| 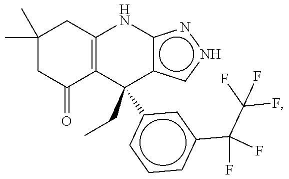 | 949103 |
| 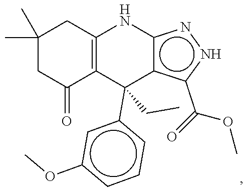 | 901479 |
| 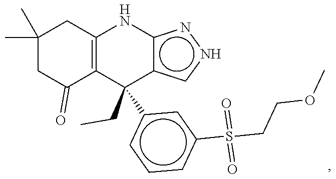 | 943542 |
| 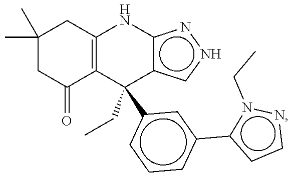 | 946089 |
| 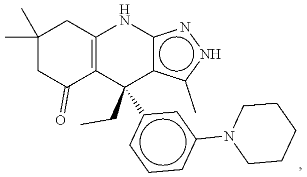 | 884116 |
| 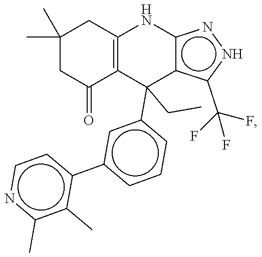 | 945529 |
| 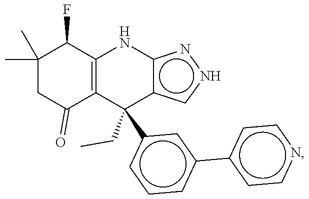 | 959343 |
| 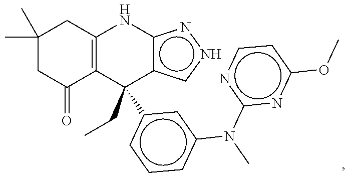 | 947336 |
| 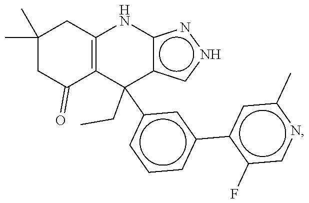 | 917132 |
| 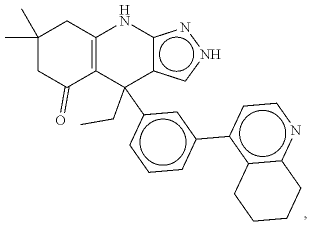 | 932625 |
| 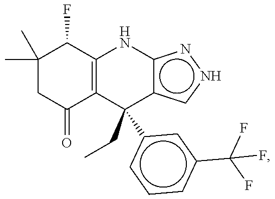 | 837646 |
| 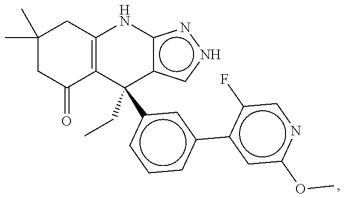 | 944032 |
| 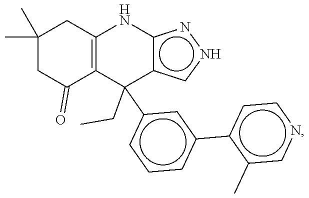 | 913351 |
| 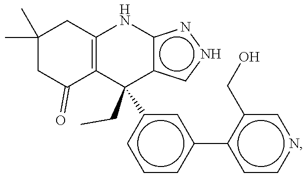 | 945469 |
| 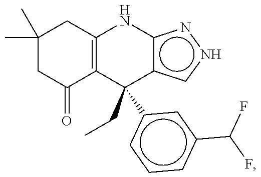 | 826717 |
| 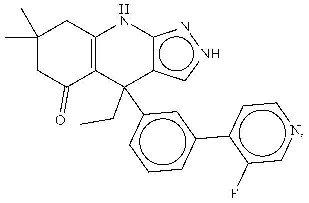 | 913349 |
| 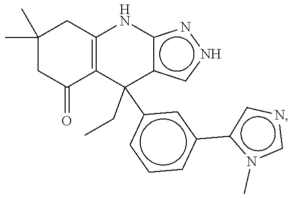 | 943533 |
| 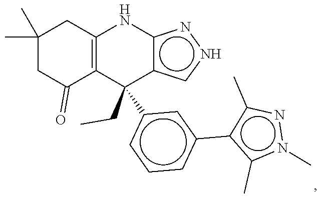 | 911990 |
| 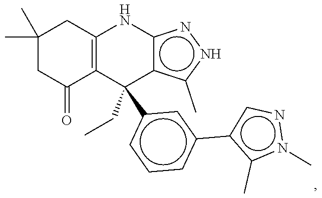 | 913045 |
| 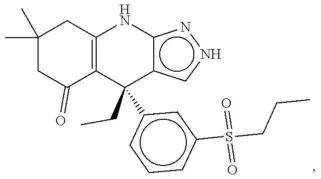 | 929447 |
| 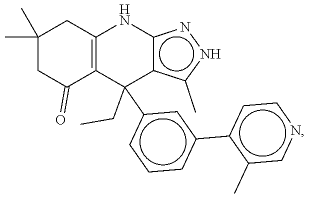 | 883991 |
| 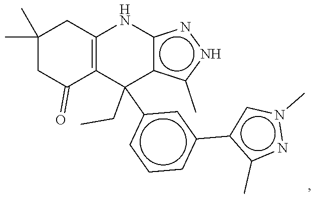 | 884003 |
| 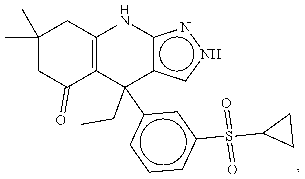 | 909204 |
| 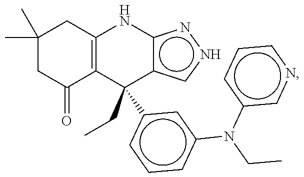 | 917088 |
| 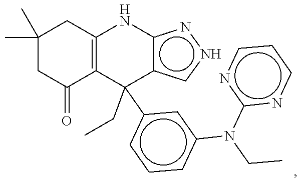 | 919840 |
| 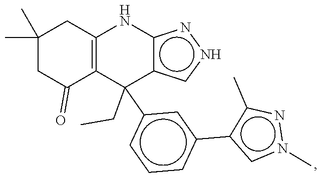 | 926840 |
| 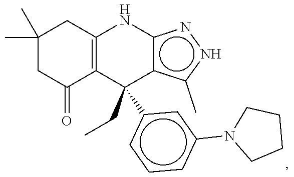 | 883874 |
| 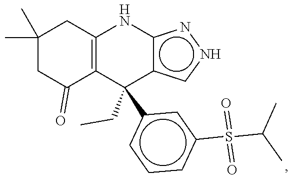 | 913135 |
| 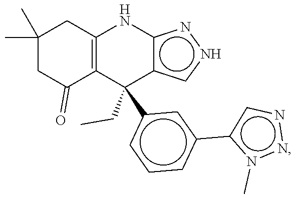 | 947802 |
| 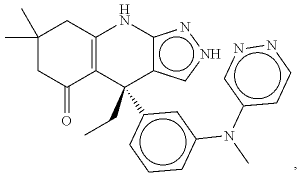 | 943492 |
| 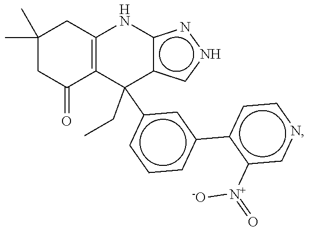 | 947631 |
| 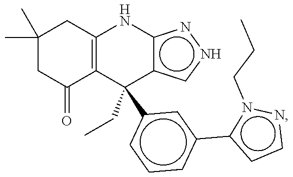 | 946093 |
| 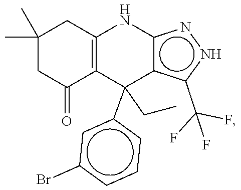 | 801324 |
| 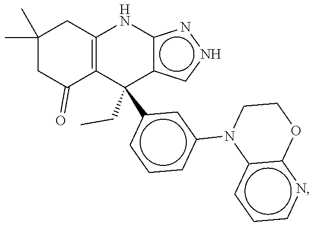 | 932663 |
| 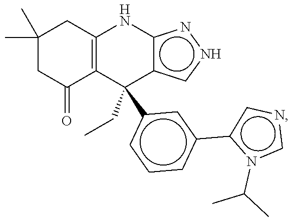 | 945725 |
| 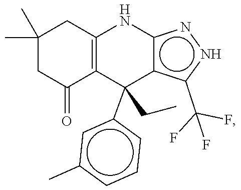 | 883873 |
| 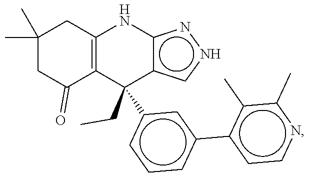 | 943516 |
| 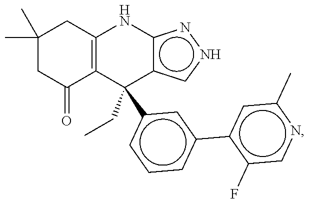 | 927346 |
| 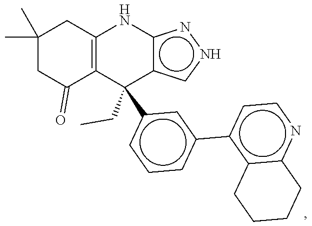 | 944033 |
| 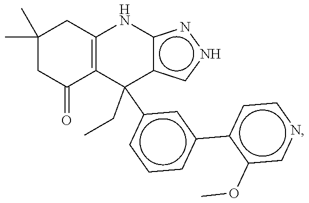 | 932627 |
| 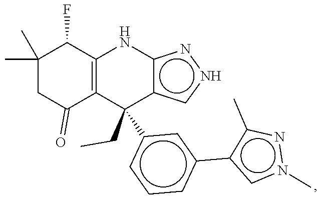 | 947651 |
| 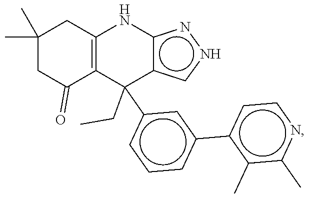 | 913347 |
| 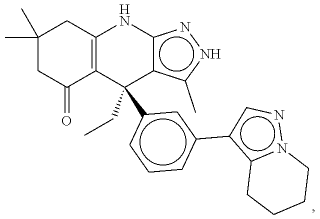 | 913036 |
| 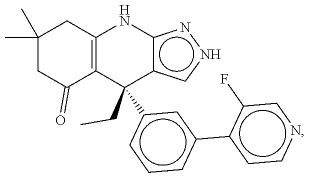 | 943518 |
| 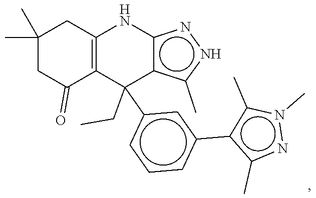 | 883995 |
| 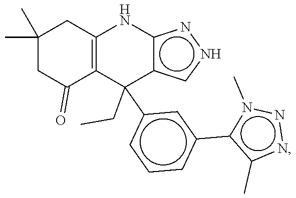 | 943996 |
| 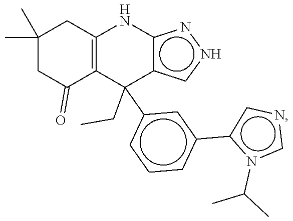 | 944026 |
| 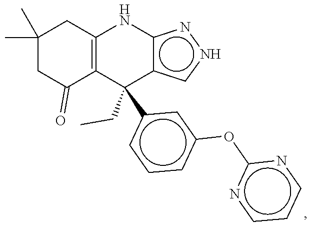 | 947808 |
| 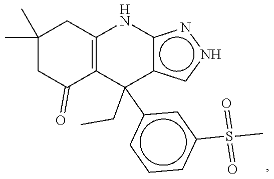 | 819561 |
| 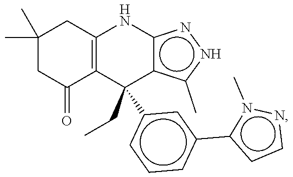 | 913023 |
| 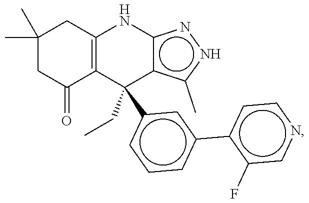 | 913052 |
| 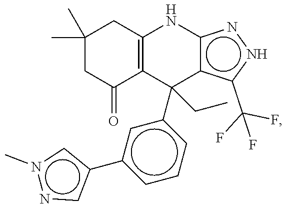 | 801445 |
| 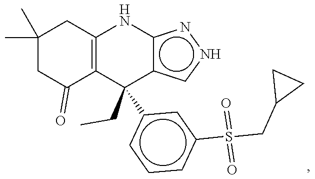 | 917150 |
| 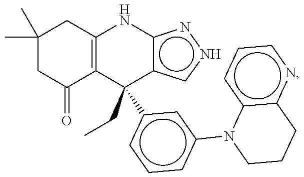 | 917076 |
| 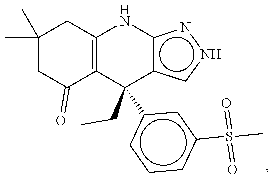 | 826718 |
| 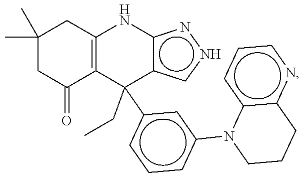 | 889639 |
| 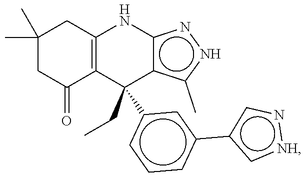 | 913025 |
| 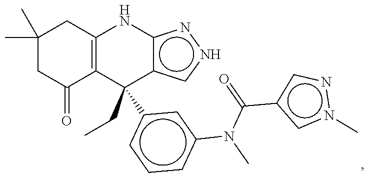 | 913311 |
| 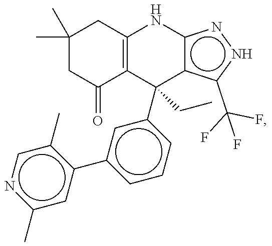 | 947363 |
| 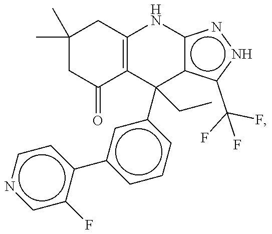 | 945528 |
| 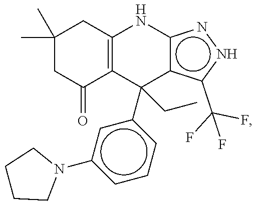 | 810506 |
| 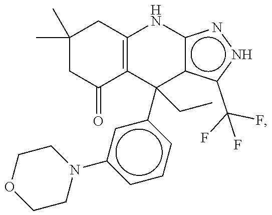 | 810507 |
| 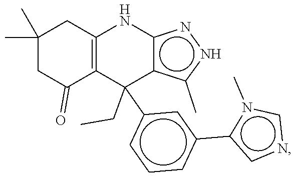 | 884001 |
| 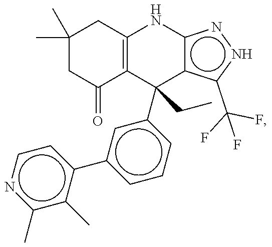 | 947329 |
| 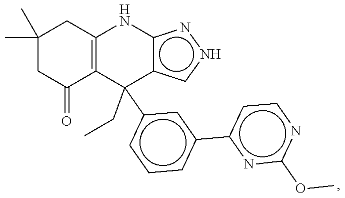 | 948092 |
| 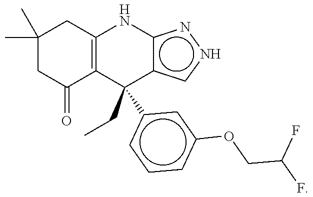 | 949104 |

In some embodiments, Formula IV-A or IV-B is any one of the formulae shown in Table 7A:

TABLE 7A

| 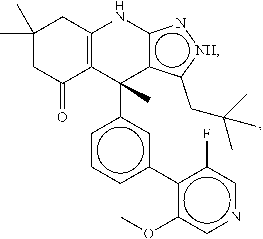 | 108954 |
|---|---|
| 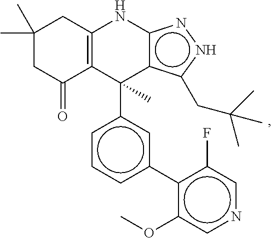 | 108956 |
| 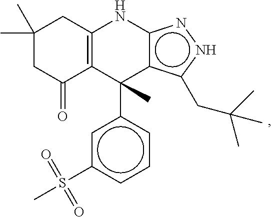 | 114687 |
| 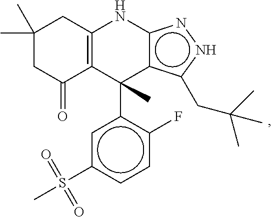 | 125976 |
| 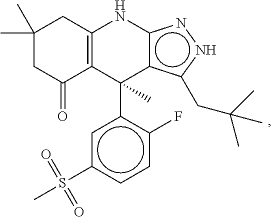 | 125977 |
| 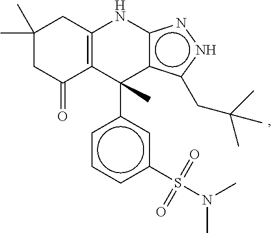 | 126012 |
| 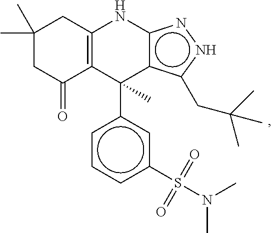 | 126013 |
| 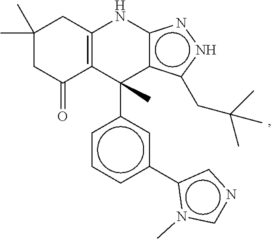 | 126014 |
| 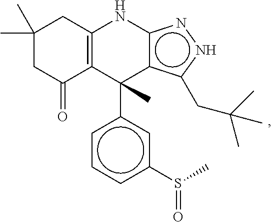 | 126190 |
| 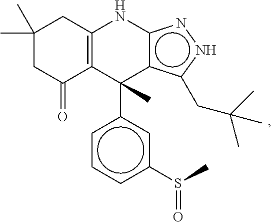 | 126191 |
| 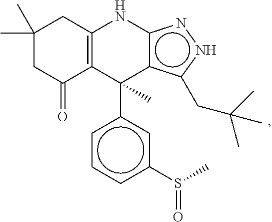 | 126192 |
| 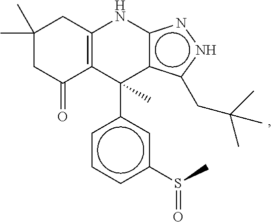 | 126193 |
| 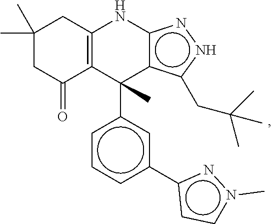 | 126366 |
| 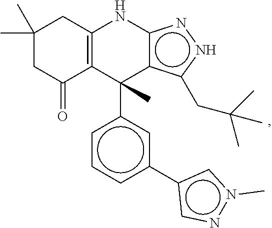 | 126393 |
| 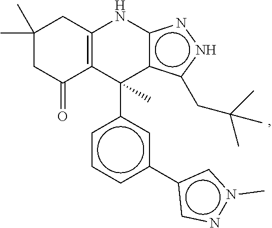 | 126394 |
| 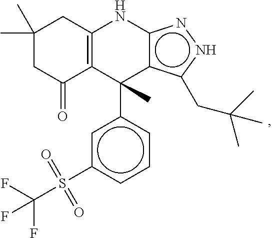 | 126395 |
| 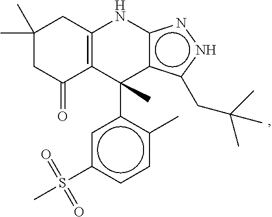 | 126397 |
| 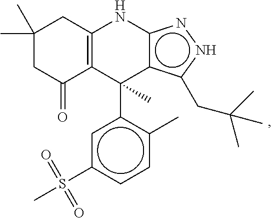 | 126398 |
| 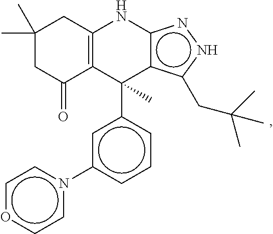 | 126399 |
| or | |
| 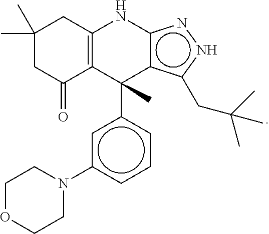 | 126400 |

In some embodiments, Formula IV-A or IV-B is any one of the formulae shown in Table 8:

TABLE 8

| | |
|---|---|
| 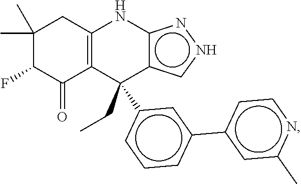 | 945485 |
| 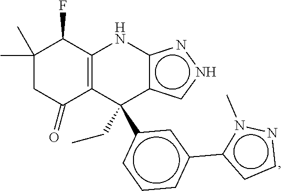 | 957508 |
| 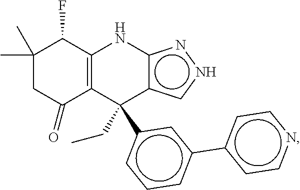 | 959344 |
| 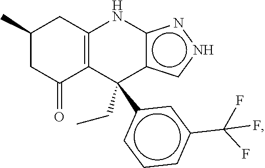 | 810453 |
| 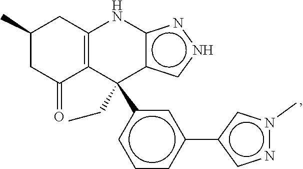 | 823726 |
| 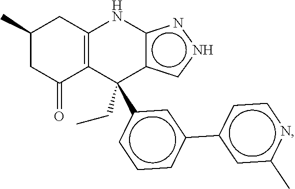 | 932637 |
| 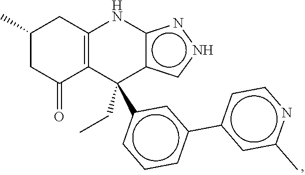 | 932638 |
| 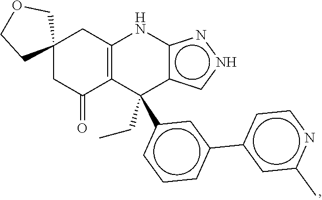 | 947411 |
| 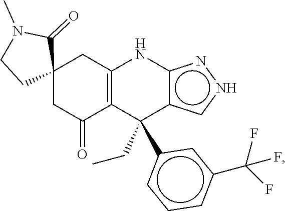 | 950325 |
| 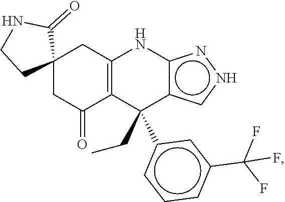 | 950332 |
| 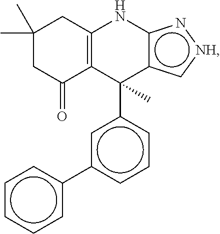 | 801366 |
| 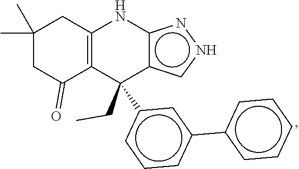 | 801369 |
| 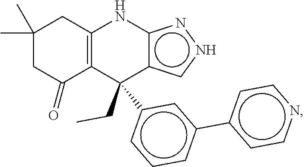 | 801412 |
| 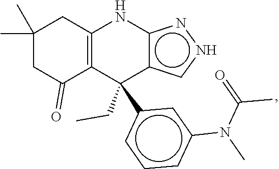 | 810359 |
| 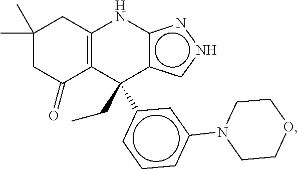 | 810364 |
| 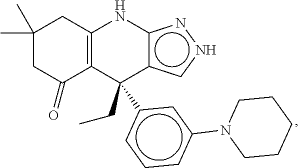 | 810371 |
| 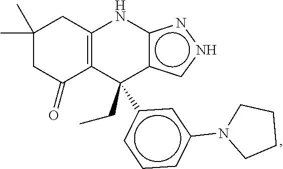 | 810509 |
| 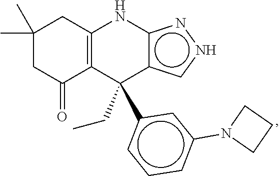 | 889632 |
| 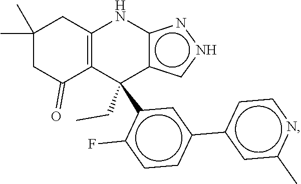 | 909213 |
| 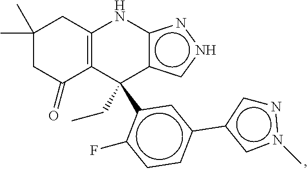 | 909223 |
| 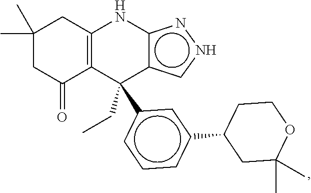 | 912091 |
| 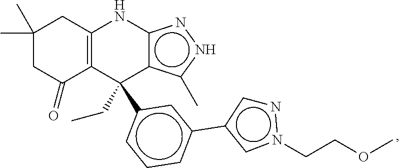 | 913042 |
| 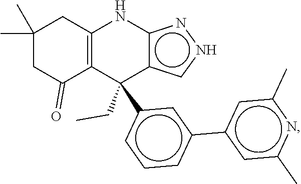 | 913223 |
| 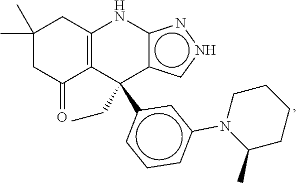 | 917210 |
| 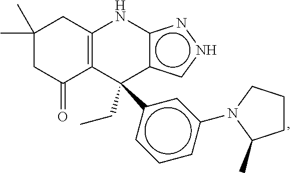 | 918322 |
| 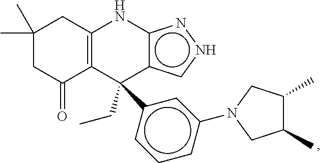 | 924520 |
| 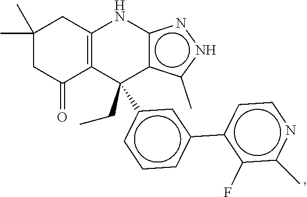 | 927340 |
| 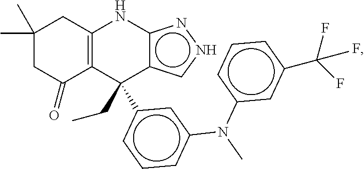 | 927345 |
| 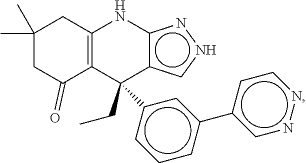 | 929443 |
| 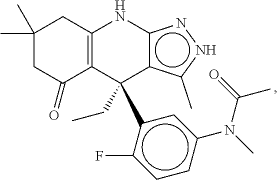 | 929451 |
| 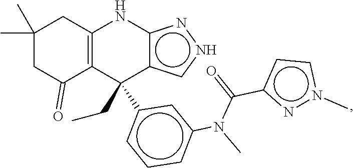 | 929455 |
| 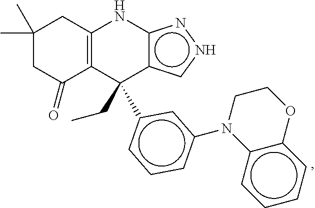 | 932635 |
| 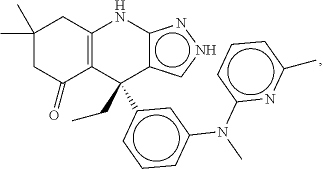 | 932644 |
| 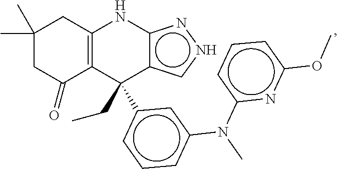 | 932646 |
| 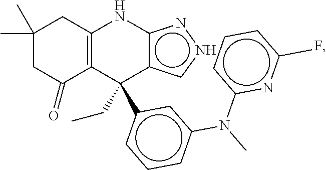 | 932656 |
| 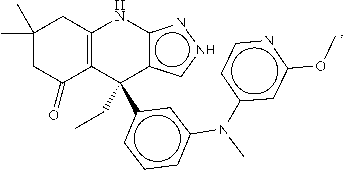 | 932662 |
| 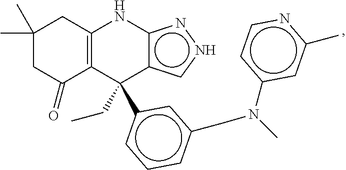 | 932665 |
| 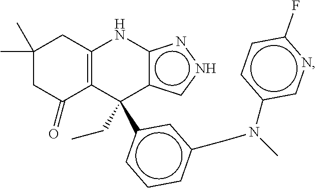 | 932667 |
| 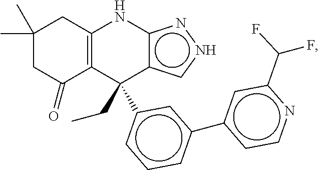 | 943915 |
| 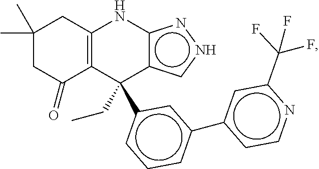 | 943989 |
| 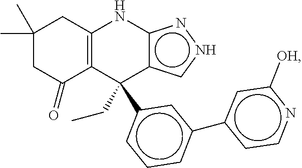 | 943993 |
| 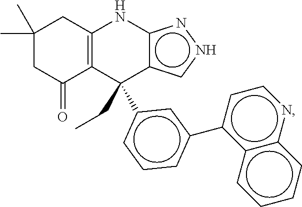 | 944034 |
| 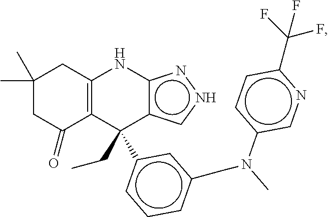 | 945484 |
| 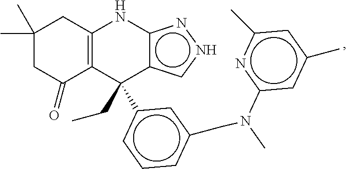 | 945615 |
| 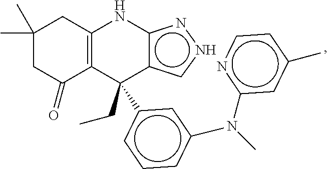 | 945616 |
| 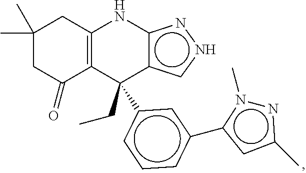 | 946000 |
| 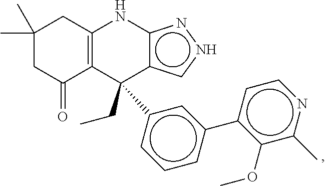 | 946002 |
| 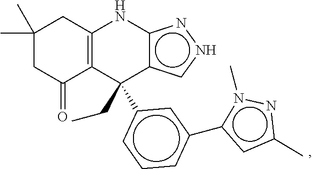 | 946098 |
| 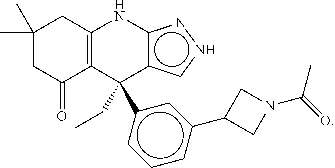 | 949132 |

In some embodiments, Formula IV-A or IV-B is any one of the formulae shown in Table 8A

TABLE 8A

| | |
|---|---|
| 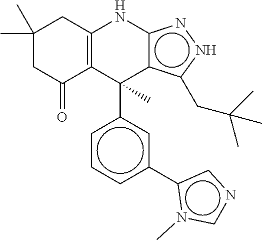 | 126015 |
| 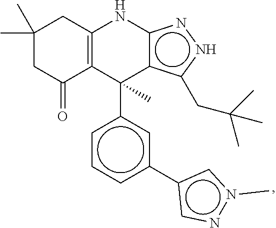 | 126367 |

or

| | |
|---|---|
| 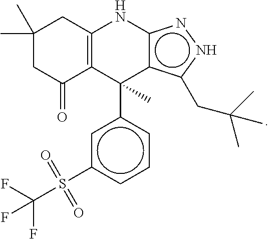 | 126396 |

In some embodiments, Formula IV-A or IV-B is not any one of the formulae shown in Table 8. In some embodiments, Formula IV-A or IV-B is not any one of the formulae shown in Table 8A.

In another aspect, provided herein is a compound, or a pharmaceutically acceptable salt, solvate, hydrate, polymorph, co-crystal, tautomer, stereoisomer, isotopically labeled compound, or prodrug thereof, wherein the compound is of Formula V-A or V-B:

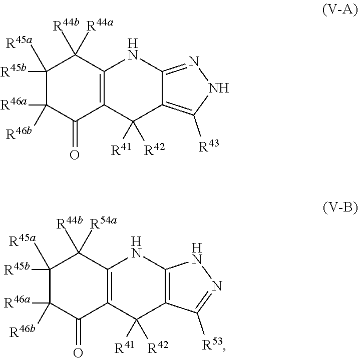

wherein:
R⁴¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R⁴² is optionally substituted alkyl, optionally substituted alkenyl, or optionally substituted alkynyl;
or R⁴¹ and R⁴² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
R⁴³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{E}, —SCN, —SR^{E}, —SSR^{E}, —N₃, —NO, —N(R^{E})₂, —NO₂, —C(═O)R^{E}, —C(═O)OR^{E}, —C(═O)SR^{E}, —C(═O)N(R^{E})₂, —C(═NR^{E})R^{E}, —C(═NR^{E})OR^{E}, —C(═NR^{E})SR^{E}, —C(═NR^{E})N(R^{E})₂, —S(═O)R^{E}, —S(═O)OR^{E}, —S(═O)SR^{E}, —S(═O)N(R^{E})₂, —S(═O)₂R^{E}, —S(═O)₂OR^{E}, —S(═O)₂SR^{E}, —S(═O)₂N(R^{E})₂, —OC(═O)R^{E}, —OC(═O)OR^{E}, —OC(═O)SR^{E}, —OC(═O)N(R^{E})₂, —OC(NR^{E})R^{E}, —OC(═NR^{E})OR^{E}, —OC(═NR^{E})SR^{E}, —OC(═NR^{E})N(R^{E})₂, —OS(═O)R^{E}, —OS(═O)OR^{E}, —OS(═O)SR^{E}, —OS(═O)N(R^{E})₂, —OS(═O)₂R^{E}, —OS(═O)₂OR^{E}, —OS(═O)₂SR^{E}, —OS(═O)₂N(R^{E})₂, —ON(R^{E})₂, —SC(═O)R^{E}, —SC(O)OR^{E}, —SC(═O)SR^{E}, —SC(═O)N(R^{E})₂, —SC(—NR^{E})R^{E}, —SC(—NR^{E})OR^{E}, —SC(═NR^{E})SR^{E}, —SC(═NR^{E})N(R^{E})₂, —NR^{E}C(═O)R^{E}, —NR^{E}C(═O)OR^{E}, —NR^{E}C(═O)SR^{E}, —NR^{E}C(═O)N(R^{E})₂, —NR^{E}C(═NR^{E})R^{E}, —NR^{E}C(═NR^{E})OR^{E}, —NR^{E}C(═NR^{E})SR^{E}, —NR^{E}C(═NR^{E})N(R^{E})₂, —NR^{E}S(═O)R^{E}, —NR^{E}S(═O)OR^{E}, —NR^{E}S(═O)SR^{E}, —NR^{E}S(═O)N(R^{E})₂, —NR^{E}S(═O)₂R^{E}, —NR^{E}S(═O)₂OR^{E}, —NR^{E}S(═O)₂SR^{E}, —NR^{E}S(═O)₂N(R^{E})₂, —Si(R^{E})₂, —Si(R^{E})₂OR^{E}, —Si(R^{E})(OR^{E})₂, —Si(OR^{E})₃, —OSi(R^{E})₃, —OSi(R^{E})₂OR^{E}, —OSi(R^{E})(OR^{E})₂, or —OSi(OR^{E})₃;
each of R^{44a}, R^{44b}, R^{45a}, R^{45b}, R^{46a}, and R^{46b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or —OR^{E}; and
each instance of R^{E} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{E} attached to the same intervening atom are joined together with the intervening atom to form an optionally substituted, monocyclic, heterocyclic or heteroaryl ring;
or R^{44a} and R^{44b} are taken together with their intervening atom to form an optionally substituted, monocyclic, carbocyclic or heterocyclic ring;
or R^{44b} and R^{45a} are taken together with their intervening atom to form an optionally substituted, monocyclic, carbocyclic or heterocyclic ring;
or R^{45a} and R^{45b} are taken together with their intervening atom to form an optionally substituted, monocyclic, carbocyclic or heterocyclic ring;
or R^{45b} and R^{45a} are taken together with their intervening atom to form an optionally substituted, monocyclic, carbocyclic or heterocyclic ring;
or R^{46a} and R^{46b} are taken together with their intervening atom to form an optionally substituted, monocyclic, carbocyclic or heterocyclic ring;
provided that at least one of R^{44a}, R^{44b}, R^{46a}, and R^{46b} is not hydrogen.

In some embodiments, the compound is of the formula:

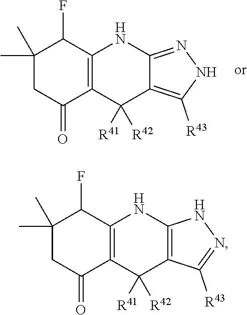

or a pharmaceutically acceptable salt, solvate, hydrate, polymorph, co-crystal, tautomer, stereoisomer, isotopically labeled compound, or prodrug thereof.

In some embodiments, the compound is of the formula:

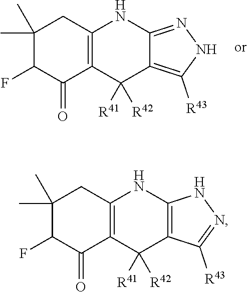

or a pharmaceutically acceptable salt, solvate, hydrate, polymorph, co-crystal, tautomer, stereoisomer, isotopically labeled compound, or prodrug thereof.

In some embodiments, the compound is of the formula:

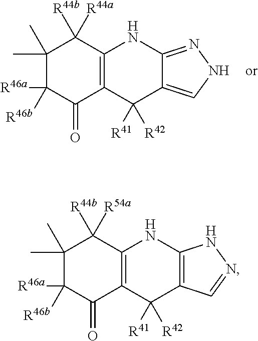

or a pharmaceutically acceptable salt, solvate, hydrate, polymorph, co-crystal, tautomer, stereoisomer, isotopically labeled compound, or prodrug thereof,
wherein:
at least one of R^{44a}, R^{44b}, R^{46a}, and R^{46b} is halogen.

In some embodiments, the compound is of the formula:

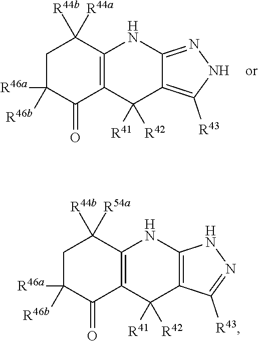

or a pharmaceutically acceptable salt, solvate, hydrate, polymorph, co-crystal, tautomer, stereoisomer, isotopically labeled compound, or prodrug thereof,
wherein:
at least two of R^{44a}, R^{44b}, R^{46a}, and R^{46b} are optionally substituted alkyl, optionally substituted alkenyl, or optionally substituted alkynyl.

In some embodiments,

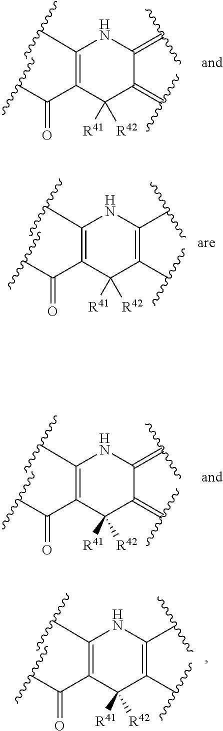

respectively. In some embodiments,

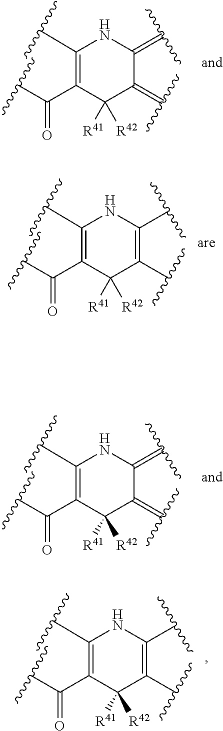

respectively.

In some embodiments, R⁴¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl. In some embodiments, R⁴¹ is optionally substituted C₁₋₁₀ alkyl, optionally substituted C₁₋₁₀ alkenyl, or optionally substituted C₁₋₁₀ alkynyl.

In some embodiments, R⁴¹ is optionally substituted carbocyclyl or optionally substituted heterocyclyl. In some embodiments, R⁴¹ is optionally substituted C₃₋₁₄ carbocyclyl. In some embodiments, R⁴¹ is optionally substituted monocyclic C₃₋₇ carbocyclyl. In some embodiments, R⁴¹ is optionally substituted monocyclic C₃₋₄ carbocyclyl. In some embodiments, R⁴¹ is optionally substituted monocyclic C₅₋₇ carbocyclyl. In some embodiments, R⁴¹ is saturated carbocyclyl. In some embodiments, R⁴¹ is carbocyclyl comprising only one unsaturated bond (e.g., C═C bond) in the carbocyclic ring system.

In some embodiments, R⁴¹ is 3- to 14-membered optionally substituted heterocyclyl. In some embodiments, R⁴¹ is optionally substituted monocyclic 3- to 7-membered heterocyclyl. In some embodiments, R⁴¹ is optionally substituted 3- to 14-membered heterocyclyl comprising one or more O and/or S atoms but no N atoms. In some embodiments, R⁴¹ is optionally substituted 3- to 14-membered heterocyclyl comprising one or more N atoms and optionally one or more O and/S atoms. In some embodiments, R⁴¹ is optionally substituted monocyclic 3- to 7-membered heterocyclyl comprising one or more O and/or S atoms but no N atoms. In some embodiments, R⁴¹ is optionally substituted monocyclic 3- to 7-membered heterocyclyl comprising one or more N atoms and optionally one or more O and/or S atoms. In some embodiments, R⁴¹ is saturated heterocyclyl. In some embodiments, R⁴¹ is heterocyclyl comprising only one unsaturated bond (e.g., C═C bond) in the heterocyclic ring system.

In some embodiments, R⁴¹ is optionally substituted aryl. In some embodiments, R⁴¹ is optionally substituted monocyclic aryl. In some embodiments, R⁴¹ is optionally substituted bicyclic aryl. In some embodiments, R⁴¹ is optionally substituted C₆₋₁₄ aryl. In some embodiments, R⁴¹ is optionally substituted C₆₋₁₀ aryl. In some embodiments, R⁴¹ is optionally substituted phenyl. In some embodiments, R⁴¹ is optionally substituted naphthyl.

In some embodiments, R⁴¹ is optionally substituted heteroaryl. In some embodiments, R⁴¹ is optionally substituted monocyclic heteroaryl. In some embodiments, R⁴¹ is optionally substituted bicyclic heteroaryl. In some embodiments, R⁴¹ is optionally substituted 5- to 14-membered heteroaryl. In some embodiments, R⁴¹ is optionally substituted 5- to 10-membered heteroaryl. In some embodiments, R⁴¹ is optionally substituted 5- to 6-membered monocyclic heteroaryl. In some embodiments, R⁴¹ is optionally substituted 9- to 10-membered bicyclic heteroaryl.

In some embodiments, R⁴¹ is optionally substituted aryl fused with optionally substituted monocyclic C₃₋₇ carbocyclyl. In some embodiments, R⁴¹ is optionally substituted C₆₋₁₄ aryl fused with optionally substituted monocyclic C₃₋₇ carbocyclyl. In some embodiments, R⁴¹ is optionally substituted C₆₋₁₀ aryl fused with optionally substituted monocyclic C₃₋₇ carbocyclyl. In some embodiments, R⁴¹ is optionally substituted phenyl fused with optionally substituted monocyclic C₃₋₇ carbocyclyl. In some embodiments, R⁴¹ is optionally substituted naphthyl fused with optionally substituted monocyclic C₃₋₇ carbocyclyl.

In some embodiments, R⁴¹ is optionally substituted aryl fused with optionally substituted monocyclic 3- to 7-membered heterocyclyl. In some embodiments, R⁴¹ is optionally substituted C₆₋₁₄ aryl fused with optionally substituted monocyclic 3- to 7-membered heterocyclyl. In some embodiments, R⁴¹ is optionally substituted C₆₋₁₀ aryl fused with optionally substituted monocyclic 3- to 7-membered heterocyclyl. In some embodiments, R⁴¹ is optionally substituted phenyl fused with optionally substituted monocyclic 3- to 7-membered heterocyclyl. In some embodiments, R⁴¹ is optionally substituted naphthyl fused with optionally substituted monocyclic 3- to 7-membered heterocyclyl.

In some embodiments, R⁴¹ is optionally substituted heteroaryl fused with optionally substituted monocyclic C₃₋₇ carbocyclyl. In some embodiments, R⁴¹ is optionally substituted 5- to 14-membered heteroaryl fused with optionally substituted monocyclic C₃₋₇ carbocyclyl. In some embodiments, R⁴¹ is optionally substituted 5- to 10-membered heteroaryl fused with optionally substituted monocyclic C₃₋₇ carbocyclyl. In some embodiments, R⁴¹ is optionally substituted 5- to 6-membered monocyclic heteroaryl fused with optionally substituted monocyclic C₃₋₇ carbocyclyl. In some embodiments, R⁴¹ is optionally substituted 9- to 10-membered bicyclic heteroaryl fused with optionally substituted monocyclic C₃₋₇ carbocyclyl.

In some embodiments, R⁴¹ is optionally substituted heteroaryl fused with optionally substituted monocyclic 3- to 7-membered heterocyclyl. In some embodiments, R⁴¹ is optionally substituted 5- to 14-membered heteroaryl fused with optionally substituted monocyclic 3- to 7-membered heterocyclyl. In some embodiments, R⁴¹ is optionally substituted 5- to 10-membered heteroaryl fused with optionally substituted monocyclic 3- to 7-membered heterocyclyl. In some embodiments, R⁴¹ is optionally substituted 5- to 6-membered monocyclic heteroaryl fused with optionally substituted monocyclic 3- to 7-membered heterocyclyl. In some embodiments, R⁴¹ is optionally substituted 9- to 10-membered bicyclic heteroaryl fused with optionally substituted monocyclic 3- to 7-membered heterocyclyl.

In some embodiments, R⁴¹ is

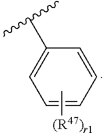

Each instance of R⁴⁷ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃.

r1 is 0, 1, 2, 3, 4, or 5. In some embodiments, r1 is 0, 1, 2, 3, or 4. In some embodiments, r1 is 0, 1, 2, or 3. In some embodiments, r1 is 0, 1, or 2. In some embodiments, r1 is 0 or 1. In some embodiments, r1 is 0. In some embodiments, r1 is 1. In some embodiments, r1 is 2. In some embodiments, r1 is 3. In some embodiments, r1 is 4. In some embodiments, r1 is 5.

In some embodiments, R⁴¹ is

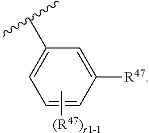

In some embodiments, R⁴¹ is

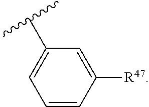

In some embodiments, R⁴¹ is

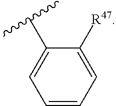

In some embodiments, R⁴¹ is

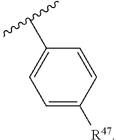

In some embodiments, R⁴¹ is

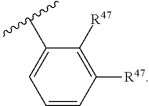

In some embodiments, R⁴¹ is

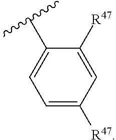

In some embodiments, R⁴¹ is

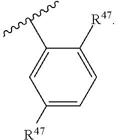

In some embodiments, R⁴¹ is

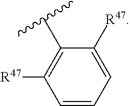

In some embodiments, R⁴¹ is

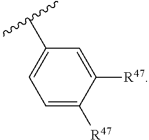

In some embodiments, R⁴¹ is

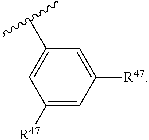

In some embodiments, at least one instance of R⁴⁷ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{D}, —SR^{D}, or —N(R^{D})₂. In some embodiments, at least one instance of R⁴⁷ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, at least one instance of R⁴⁷ is C₁₋₁₀ haloalkyl. In some embodiments, at least one instance of R⁴⁷ is C₁₋₄ haloalkyl. In some embodiments, at least one instance of R⁴⁷ is C₁₋₄ fluoroalkyl (e.g., C₁₋₄ perfluoroalkyl). In some embodiments, at least one instance of R⁴⁷ is —CF₃.

In some embodiments, at least one instance of R⁴⁷ is halogen. In some embodiments, at least one instance of R⁴⁷ is bromine, chlorine, or fluorine. In some embodiments, at least one instance of R⁴⁷ is bromine or chlorine. In some embodiments, at least one instance of R⁴⁷ is chlorine or fluorine. In some embodiments, at least one instance of R⁴⁷ is bromine. In some embodiments, at least one instance of R⁴⁷ is chlorine. In some embodiments, at least one instance of R⁴⁷ is fluorine.

In some embodiments, at least one instance of R⁴⁷ is —OR^{D}, —SR^{D}, or —N(R^{D})₂ (e.g., wherein R^{D} is hydrogen or optionally substituted alkyl, or optionally substituted phenyl). In some embodiments, at least one instance of R⁴⁷ is —OH. In some embodiments, at least one instance of R⁴⁷ is —SH. In some embodiments, at least one instance of R⁴⁷ is —NH₂. In some embodiments, at least one instance of R⁴⁷ is —CN, —SCN, —SSR^{D}, —N₃, —NO, or —NO₂. In some embodiments, at least one instance of R⁴⁷ is —C(═O)R^{D}, —C(═O)OR^{D}, —C(═O)SR^{D}, or —C(═O)N(R²)₂ (e.g., wherein R^{D} is hydrogen or optionally substituted alkyl, or optionally substituted phenyl). In some embodiments, at least one instance of R⁴⁷ is —C(═NR^{D})R^{D}, —C(═NR^{D})OR^{D}, —C(═NR^{D})SR^{D}, or —C(═NR^{D})N(R^{D})₂ (e.g., wherein R^{D} is hydrogen or optionally substituted alkyl, or optionally substituted phenyl). In some embodiments, at least one instance of R⁴⁷ is —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, or —S(═O)₂N(R^{D})₂ (e.g., wherein R^{D} is hydrogen or optionally substituted alkyl, or optionally substituted phenyl). In some embodiments, at least one instance of R⁴⁷ is —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, or —ON(R^{D})₂ (e.g., wherein R^{D} is hydrogen or optionally substituted alkyl, or optionally substituted phenyl). In some embodiments, at least one instance of R⁴⁷ is —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, or —SC(═NR^{D})N(R^{D})₂ (e.g., wherein RP is hydrogen or optionally substituted alkyl, or optionally substituted phenyl). In some embodiments, at least one instance of R⁴⁷ is —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂RP, —NR^{D}S(═O)₂OR^{D}, —NR^{D}S(═O)₂SR^{D}, or —NR^{D}S(═O)₂N(R^{D})₂ (e.g., wherein R^{D} is hydrogen or optionally substituted alkyl, or optionally substituted phenyl). In some embodiments, at least one instance of R⁴⁷ is —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃ (e.g., wherein R^{D} is hydrogen or optionally substituted alkyl, or optionally substituted phenyl).

In some embodiments, at least one instance of R⁴⁷ is optionally substituted carbocyclyl, optionally substituted heterocyclyl, optionally substituted aryl, or optionally substituted heteroaryl. In some embodiments, at least one instance of R⁴⁷ is optionally substituted C₃₋₁₄ carbocyclyl. In some embodiments, at least one instance of R⁴⁷ is optionally substituted monocyclic C₃₋₇ carbocyclyl. In some embodiments, at least one instance of R⁴⁷ is optionally substituted monocyclic C₃₋₄ carbocyclyl. In some embodiments, at least one instance of R⁴⁷ is optionally substituted monocyclic C₅₋₇ carbocyclyl. In some embodiments, at least one instance of R⁴⁷ is saturated carbocyclyl. In some embodiments, at least one instance of R⁴⁷ is carbocyclyl comprising only one unsaturated bond (e.g., C═C bond) in the carbocyclic ring system.

In some embodiments, at least one instance of R⁴⁷ is optionally substituted 3- to 14-membered heterocyclyl. In some embodiments, at least one instance of R⁴⁷ is optionally substituted monocyclic 3- to 7-membered heterocyclyl. In some embodiments, at least one instance of R⁴⁷ is optionally substituted 3- to 14-membered heterocyclyl comprising one or more O and/or S atoms but no N atoms. In some embodiments, at least one instance of R⁴⁷ is optionally substituted 3- to 14-membered heterocyclyl comprising one or more N atoms and optionally one or more O and/S atoms. In some embodiments, at least one instance of R⁴⁷ is optionally substituted monocyclic 3- to 7-membered heterocyclyl comprising one or more O and/or S atoms but no N atoms. In some embodiments, at least one instance of R⁴⁷ is optionally substituted monocyclic 3- to 7-membered heterocyclyl comprising one or more N atoms and optionally one or more O and/or S atoms. In some embodiments, at least one instance of R⁴⁷ is saturated heterocyclyl. In some embodiments, at least one instance of R⁴⁷ is heterocyclyl comprising only one unsaturated bond (e.g., C═C bond) in the heterocyclic ring system.

In some embodiments, at least one instance of R⁴⁷ is optionally substituted monocyclic aryl. In some embodiments, at least one instance of R⁴⁷ is optionally substituted bicyclic aryl. In some embodiments, at least one instance of R⁴⁷ is optionally substituted C₆₋₁₄ aryl. In some embodiments, at least one instance of R⁴⁷ is optionally substituted C₆₋₁₀ aryl. In some embodiments, at least one instance of R⁴⁷ is optionally substituted phenyl. In some embodiments, at least one instance of R⁴⁷ is optionally substituted naphthyl.

In some embodiments, at least one instance of R⁴⁷ is optionally substituted monocyclic heteroaryl. In some embodiments, at least one instance of R⁴⁷ is optionally substituted bicyclic heteroaryl. In some embodiments, at least one instance of R⁴⁷ is optionally substituted 5- to 14-membered heteroaryl. In some embodiments, at least one instance of R⁴⁷ is optionally substituted 5- to 10-membered heteroaryl. In some embodiments, at least one instance of R⁴⁷ is optionally substituted 5- to 6-membered monocyclic heteroaryl. In some embodiments, at least one instance of R⁴⁷ is optionally substituted heteroaryl comprising one or more N atoms. In some embodiments, at least one instance of R⁴⁷ is optionally substituted pyridinyl, optionally substituted imidazolyl, or optionally substituted pyrazolyl.

In some embodiments, at least one instance of R^{D} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{D} is optionally substituted carbocyclyl, optionally substituted heterocyclyl, optionally substituted aryl, or optionally substituted heteroaryl. In some embodiments, at least one instance of R^{D} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{D} is independently hydrogen, optionally substituted C₁₋₁₀ alkyl, or optionally substituted phenyl. In some embodiments, at least one instance of R^{D} is a nitrogen protecting group when attached to a nitrogen atom, an oxygen protecting group when attached to an oxygen atom, or a sulfur protecting group when attached to a sulfur atom. In some embodiments, two instances of R^{D} attached to the same intervening atom are joined together with the intervening atom to form an optionally substituted, monocyclic, heterocyclic or heteroaryl ring.

In some embodiments, R⁴² is optionally substituted alkyl, optionally substituted alkenyl, or optionally substituted alkynyl. In some embodiments, R⁴² is optionally substituted C₁₋₄ alkyl, optionally substituted C₁₋₄ alkenyl, or optionally substituted C₁₋₄ alkynyl. In some embodiments, R⁴² is optionally substituted alkyl. In some embodiments, R⁴² is optionally substituted C₁₋₄ alkyl. In some embodiments, R⁴² is unsubstituted C₁-C₄ alkyl. In some embodiments, R⁴² is unsubstituted methyl. In some embodiments, R⁴² is unsubstituted ethyl.

In some embodiments, R⁴¹ and R⁴² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring. In some embodiments, R⁴¹ and R⁴² are taken together with their intervening atom to form an optionally substituted, monocyclic, carbocyclic ring. In some embodiments, R⁴¹ and R⁴² are taken together with their intervening atom to form an optionally substituted, monocyclic, 3- to 7-membered carbocyclic ring. In some embodiments, R⁴¹ and R⁴² are taken together with their intervening atom to form an optionally substituted, monocyclic, 5- to 6-membered carbocyclic ring. In some embodiments, R⁴¹ and R⁴² are taken together with their intervening atom to form an optionally substituted, monocyclic, heterocyclic ring. In some embodiments, R⁴¹ and R⁴² are taken together with their intervening atom to form an optionally substituted, monocyclic, 3- to 7-membered heterocyclic ring. In some embodiments, R⁴¹ and R⁴² are taken together with their intervening atom to form an optionally substituted, monocyclic, 5- to 6-membered heterocyclic ring. In some embodiments, R⁴¹ and R⁴² are taken together with their intervening atom to form an optionally substituted, monocyclic, heterocyclic ring comprising one or more N atoms. In some embodiments, R⁴¹ and R⁴² are taken together with their intervening atom to form an optionally substituted, monocyclic, 3- to 7-membered heterocyclic ring comprising one or more N atoms. In some embodiments, R⁴¹ and R⁴² are taken together with their intervening atom to form an optionally substituted cyclohexyl, piperidinyl, or tetrahydropyranyl.

In some embodiments, R⁴³ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{E}, —SR^{E}, or —N(R^{E})₂. In some embodiments, R⁴³ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R⁴³ is C₁₋₁₀ haloalkyl. In some embodiments, R⁴³ is C₁₋₄ haloalkyl. In some embodiments, R⁴³ is C₁₋₄ fluoroalkyl (e.g., C₁₋₄ perfluoroalkyl). In some embodiments, R⁴³ is —CF₃. In some embodiments, R⁴³ is hydrogen, optionally substituted C₁-C₆ alkyl, or halogen. In some embodiments, R⁴³ is hydrogen, fluorine, —CH₃, —CH₂F, —CHF₂, or —CF₃. In some embodiments, R⁴³ is hydrogen, fluorine, —CH₃, or —CF₃. In some embodiments, R⁴³ is hydrogen or halogen. In some embodiments, R⁴³ is hydrogen or fluorine. In some embodiments, R⁴³ is hydrogen or optionally substituted C₁-C₆ alkyl. In some embodiments, R⁴³ is hydrogen, unsubstituted C₁-C₆ alkyl, or C₁₋₆ haloalkyl. In some embodiments, R⁴³ is hydrogen.

In some embodiments, R⁴³ is halogen. In some embodiments, R⁴³ is bromine, chlorine, or fluorine. In some embodiments, R⁴³ is bromine or chlorine. In some embodiments, R⁴³ is chlorine or fluorine. In some embodiments, R⁴³ is bromine. In some embodiments, R⁴³ is chlorine. In some embodiments. R⁴³ is fluorine.

In some embodiments, R⁴³ is —OR^{E}, —SR^{E}, or —N(R^{E})₂ (e.g., wherein R^{E} is hydrogen or optionally substituted alkyl, or optionally substituted phenyl). In some embodiments, R⁴³ is —OH. In some embodiments, R⁴³ is —SH. In some embodiments, R⁴³ is —NH₂. In some embodiments, R⁴³ is —CN, —SCN, —SSR^{E}, —N₃, —NO, or —NO₂. In some embodiments, R⁴³ is —C(═O)R^{E}, —C(═O)OR^{E}, —C(═O)SR^{E}, or —C(═O)N(R^{E})₂ (e.g., wherein R^{E} is hydrogen or optionally substituted alkyl, or optionally substituted phenyl). In some embodiments, R⁴³ is —C(═NR^{E})R^{E}, —C(═NR^{E})OR^{E}, —C(═NR^{E})SR^{E}, or —C(═NR^{E})N(R^{E})₂ (e.g., wherein R^{E} is hydrogen or optionally substituted alkyl, or optionally substituted phenyl). In some embodiments, R⁴³ is —S(═O)R^{E}, —S(═O)OR^{E}, —S(═O)SR^{E}, —S(═O)N(R^{E})₂, —S(═O)₂R^{E}, —S(═O)₂OR^{E}, —S(═O)₂SR^{E}, or —S(═O)₂N(R^{E})₂ (e.g., wherein R^{E} is hydrogen or optionally substituted alkyl, or optionally substituted phenyl). In some embodiments, R⁴³ is —OC(═O)R^{E}, —OC(═O)OR^{E}, —OC(═O)SR^{E}, —OC(═O)N(R^{E})₂, —OC(—NR^{E})R^{E}, —OC(═NR^{E})OR^{E}, —OC(═NR^{E})SR^{E}, —OC(═NR^{E})N(R^{E})₂, —OS(═O)R^{E}, —OS(═O)OR^{E}, —OS(═O)SR^{E}, —OS(═O)N(R^{E})₂, —OS(═O)₂R^{E}, —OS(═O)₂OR^{E}, —OS(═O)₂SR^{E}, —OS(═O)₂N(R^{E})₂, or —ON(R^{E})₂ (e.g., wherein R^{E} is hydrogen or optionally substituted alkyl, or optionally substituted phenyl). In some embodiments, R⁴³ is —SC(═O)R^{E}, —SC(═O)OR^{E}, —SC(═O)SR^{E}, —SC(═O)N(R^{E})₂, —SC(═NR^{E})R^{E}, —SC(═NR^{E})OR^{E}, —SC(═NR^{E})SR^{E}, or —SC(═NR^{E})N(R^{E})₂ (e.g., wherein R^{E} is hydrogen or optionally substituted alkyl, or optionally substituted phenyl). In some embodiments, R⁴³ is —NR^{E}C(═O)R^{E}, —NR^{E}C(═O)OR^{E}, —NR^{E}C(═O)SR^{E}, —NR^{E}C(═O)N(R^{E})₂, —NR^{E}C(═NR^{E})R^{E}, —NR^{E}C(═NR^{E})OR^{E}, —NR^{E}C(═NR^{E})SR^{E}, —NR^{E}C(═NR^{E})N(R^{E})₂, —NR^{E}S(═O)R^{E}, —NR^{E}S(═O)OR^{E}, —NR^{E}S(═O)SR^{E}, —NR^{E}S(═O)N(R^{E})₂, —NR^{E}S(═O)₂R^{E}, —NR^{E}S(═O)₂OR^{E}, —NR^{E}S(═O)₂SR^{E}, or —NR^{E}S(═O)₂N(R^{E})₂ (e.g., wherein R^{E} is hydrogen or optionally substituted alkyl, or optionally substituted phenyl). In some embodiments, R⁴³ is —Si(R^{E})₃, —Si(R^{E})₂OR^{E}, —Si(R^{E})(OR^{E})₂, —Si(OR^{E})₃, —OSi(R^{E})₃, —OSi(R^{E})₂OR^{E}, —OSi(R^{E})(OR^{E})₂, or —OSi(OR^{E})₃ (e.g., wherein R^{E} is hydrogen or optionally substituted alkyl, or optionally substituted phenyl).

In some embodiments, R⁴³ is optionally substituted carbocyclyl, optionally substituted heterocyclyl, optionally substituted aryl, or optionally substituted heteroaryl. In some embodiments, R⁴³ is optionally substituted C₃₋₁₄ carbocyclyl. In some embodiments, R⁴³ is optionally substituted monocyclic C₃₋₇ carbocyclyl. In some embodiments, R⁴³ is optionally substituted monocyclic C₃₋₄ carbocyclyl. In some embodiments, R⁴³ is optionally substituted monocyclic C₅₋₇ carbocyclyl. In some embodiments, R⁴³ is saturated carbocyclyl. In some embodiments, R⁴³ is carbocyclyl comprising only one unsaturated bond (e.g., C═C bond) in the carbocyclic ring system.

In some embodiments, R⁴³ is optionally substituted 3- to 14-membered heterocyclyl. In some embodiments, R⁴³ is optionally substituted monocyclic 3- to 7-membered heterocyclyl. In some embodiments, R⁴³ is optionally substituted 3- to 14-membered heterocyclyl comprising one or more O and/or S atoms but no N atoms. In some embodiments, R⁴³ is optionally substituted 3-12 to 14-membered heterocyclyl comprising one or more N atoms and optionally one or more O and/S atoms. In some embodiments, R⁴³ is optionally substituted monocyclic 3- to 7-membered heterocyclyl comprising one or more O and/or S atoms but no N atoms. In some embodiments, R⁴³ is optionally substituted monocyclic 3- to 7-membered heterocyclyl comprising one or more N atoms and optionally one or more O and/or S atoms. In some embodiments, R⁴³ is saturated heterocyclyl. In some embodiments, R⁴³ is heterocyclyl comprising only one unsaturated bond (e.g., C═C bond) in the heterocyclic ring system.

In some embodiments, R⁴³ is optionally substituted monocyclic aryl. In some embodiments, R⁴³ is optionally substituted bicyclic aryl. In some embodiments, R⁴³ is optionally substituted C₆₋₁₄ aryl. In some embodiments, R⁴³ is optionally substituted C₆₋₁₀ aryl. In some embodiments, R⁴³ is optionally substituted phenyl. In some embodiments, R⁴³ is optionally substituted naphthyl.

In some embodiments, R⁴³ is optionally substituted monocyclic heteroaryl. In some embodiments, R⁴³ is optionally substituted bicyclic heteroaryl. In some embodiments, R⁴³ is optionally substituted 5- to 14-membered heteroaryl. In some embodiments, R⁴³ is optionally substituted 5- to 10-membered heteroaryl. In some embodiments, R⁴³ is optionally substituted 5- to 6-membered monocyclic heteroaryl.

In some embodiments, at least one instance of R^{E} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{E} is optionally substituted carbocyclyl, optionally substituted heterocyclyl, optionally substituted aryl, or optionally substituted heteroaryl. In some embodiments, at least one instance of R^{E} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{E} is independently hydrogen, optionally substituted C₁₋₁₀ alkyl, or optionally substituted phenyl. In some embodiments, at least one instance of R^{E} is a nitrogen protecting group when attached to a nitrogen atom, an oxygen protecting group when attached to an oxygen atom, or a sulfur protecting group when attached to a sulfur atom. In some embodiments, two instances of R^{E} attached to the same intervening atom are joined together with the intervening atom to form an optionally substituted, monocyclic, heterocyclic or heteroaryl ring.

In some embodiments,

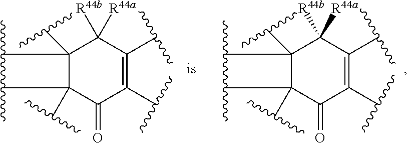

wherein R^{44a} is hydrogen, and R^{44b} is not hydrogen. In some embodiments,

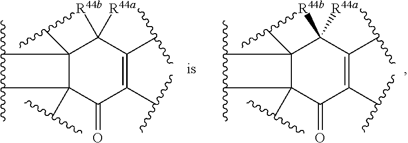

wherein R^{44a} is hydrogen, and R^{44b} is not hydrogen.

In some embodiments,

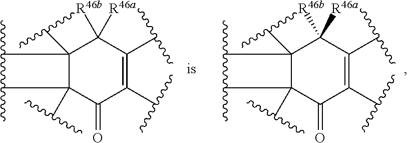

wherein R^{46a} is hydrogen, and R^{46b} is not hydrogen. In some embodiments,

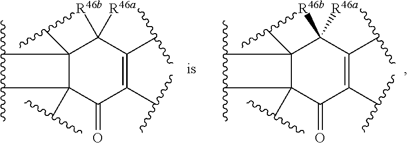

wherein R^{46a} is hydrogen, and R^{46b} is not hydrogen.

In some embodiments, one of R^{44a}, R^{44b}, R^{46a}, and R^{46b} are not hydrogen. In some embodiments, two of R^{44a}, R^{44b}, R^{46a}, and R^{46b} are not hydrogen. In some embodiments, three of R^{44a}, R^{44b}, R^{45b}, and R^{46b} are not hydrogen. In some embodiments, R^{44a}, R^{44b}, R^{46a}, and R^{46b} are not hydrogen. In some embodiments, R^{44a} is not hydrogen. In some embodiments, R^{44b} is not hydrogen. In some embodiments, R^{46a} is not hydrogen. In some embodiments, R^{46b} is not hydrogen.

In some embodiments, each of R^{44a}, R^{44b}, R^{45a}, R^{45b}, R^{46a}, and R^{46b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or —OR^{E}. In some embodiments, each of R^{44a}, R^{44b}, R^{45a}, R^{45b}, R^{46a}, and R^{46b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, each of R^{44a}, R^{44b}, R^{45a}, R^{45b}, R^{45b}, and R^{46b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, each of R^{44a}, R^{44b}, R^{45a}, R^{45b}, R^{46a}, and R^{46b} is independently hydrogen, halogen, or optionally substituted alkyl. In some embodiments, each of R^{44a}, R^{44b}, R^{45a}, R^{45b}, R^{46a}, and R^{46b} is independently hydrogen, halogen, or optionally substituted C₁₋₄ alkyl. In some embodiments, each of R^{44a}, R^{44b}, R^{45a}, R^{45b}, R^{45b}, and R^{46b} is independently hydrogen, fluorine, or optionally substituted C₁₋₄ alkyl. In some embodiments, each of R^{44a}, R^{44b}, R^{45a}, R^{45b}, R^{46a}, and R^{46b} is independently hydrogen or halogen. In some embodiments, each of R^{44a}, R^{44b}, R^{45a}, R^{45b}, R^{46a} and R^{46b} is independently hydrogen or fluorine. In some embodiments, each of R^{44a}, R^{44b}, R^{45a}, R^{45b}, R^{46a}, and R^{46b} is independently hydrogen or optionally substituted C₁₋₄ alkyl. In some embodiments, at least one of R^{44a}, R^{44b}, R^{45a}, R^{45b}, R^{46a}, and R^{46b} is hydrogen. In some embodiments, at least one of R^{44a}, R^{44b}, R^{45a}, R^{45b}, R^{46a}, and R^{46b} is optionally substituted C₁₋₄ alkyl. In some embodiments, at least one of R^{44a}, R^{44b}, R^{45a}, R^{45b}, R^{46a}, and R^{46b} is unsubstituted methyl. In some embodiments, at least one of R^{44a}, R^{44b}, R^{45a}, R^{45b}, R^{46a}, and R^{46b} is fluorine. In some embodiments, at least one of R^{44a}, R^{44b}, R^{45a}, R^{45b}, R^{46a}, and R^{46b} is —OR^{E} (e.g., wherein R^{E} is hydrogen or optionally substituted alkyl, or optionally substituted phenyl).

In some embodiments, each of R^{44a} and R^{44b} is independently hydrogen, halogen, or optionally substituted alkyl. In some embodiments, each of R^{44a} and R^{44b} is independently hydrogen, halogen, or optionally substituted C₁₋₄ alkyl. In some embodiments, at least one of R^{44a} and R^{44b} is hydrogen. In some embodiments, one of R^{44a} and R^{44b} is hydrogen. In some embodiments, R^{44a} and R^{44b} are hydrogen. In some embodiments, at least one of R^{44a} and R^{44b} is halogen. In some embodiments, one of R^{44a} and R^{44b} is halogen. In some embodiments, at least one of R^{44a} and R^{44b} is fluorine. In some embodiments, one of R^{44a} and R^{44b} is fluorine. In some embodiments, at least one of R^{44a} and R^{44b} is optionally substituted C₁₋₄ alkyl. In some embodiments, one of R^{44a} and R^{44b} is optionally substituted C₁₋₄ alkyl. In some embodiments, at least one of R^{44a} and R^{44b} is —CH₃. In some embodiments, one of R^{44a} and R^{44b} is —CH₃.

In some embodiments, each of R^{45a} and R^{45b} is independently hydrogen, halogen, or optionally substituted alkyl. In some embodiments, each of R^{45a} and R^{45b} is independently hydrogen, halogen, or optionally substituted C₁₋₄ alkyl. In some embodiments, each of R^{45a} and R^{45b} is independently hydrogen or optionally substituted C₁₋₄ alkyl. In some embodiments, at least one of R^{45a} and R^{45b} is —CH₃. In some embodiments, at least one of R^{45a} and R^{45b} is hydrogen. In some embodiments, R^{45a} and R^{45b} are —CH₃. In some embodiments, R^{45a} and R^{45b} are hydrogen.

In some embodiments, each of R^{45b} and R^{46b} is independently hydrogen, halogen, optionally substituted alkyl, or —OR^{E}. In some embodiments, each of R^{45b} and R^{46b} is independently hydrogen, halogen, or optionally substituted alkyl. In some embodiments, each of R^{46a} and R^{46b} is independently hydrogen, halogen, or optionally substituted C₁₋₄ alkyl. In some embodiments, at least one of R^{46a} and R^{46b} is hydrogen. In some embodiments, one of R^{46a} and R^{46b} is hydrogen. In some embodiments, R^{46a} and R^{46b} are hydrogen. In some embodiments, at least one of R^{46a} and R^{46b} is halogen. In some embodiments, one of R^{46a} and R^{46b} is halogen. In some embodiments, at least one of R^{46a} and R^{46b} is fluorine. In some embodiments, one of R^{45b} and R^{46b} is fluorine. In some embodiments, at least one of R^{46a} and R^{46b} is optionally substituted C₁₋₄ alkyl. In some embodiments, one of R^{46a} and R^{46b} is optionally substituted C₁₋₄ alkyl. In some embodiments, at least one of R^{46a} and R^{46b} is —CH₃. In some embodiments, one of R^{46a} and R^{46b} is —CH₃. In some embodiments, R^{46a} and R^{46b} are —CH₃. In some embodiments, one of R^{46a} and R^{46b} is —CH₃. In some embodiments, at least one of R^{45b} and R^{46b} is —OR^{E}. In some embodiments, one of R^{46a} and R^{46b} is —OR^{E}. In some embodiments, at least one of R^{46a} and R^{4b}b is —OH. In some embodiments, one of R^{46a} and R^{46b} is —OH.

In some embodiments, R^{44a} and R^{44b} are taken together with their intervening atom to form an optionally substituted, monocyclic, carbocyclic or heterocyclic ring. In some embodiments, R^{44a} and R^{44b} are taken together with their intervening atom to form an optionally substituted, monocyclic, C₃₋₁₄ carbocyclic ring. In some embodiments, R^{44a} and R^{44b} are taken together with their intervening atom to form an optionally substituted, monocyclic, C₃₋₇ carbocyclic ring. In some embodiments, R^{44a} and R^{44b} are taken together with their intervening atom to form an optionally substituted, monocyclic, 3- to 14-membered heterocyclic ring. In some embodiments, R^{44a} and R^{44b} are taken together with their intervening atom to form an optionally substituted, monocyclic, 3- to 7-membered heterocyclic ring.

In some embodiments, R^{44b} and R^{45a} are taken together with their intervening atom to form an optionally substituted, monocyclic, carbocyclic or heterocyclic ring. In some embodiments, R^{44b} and R^{45a} are taken together with their intervening atom to form an optionally substituted, monocyclic, C₃₋₁₄ carbocyclic ring. In some embodiments, R^{44b} and R^{45a} are taken together with their intervening atom to form an optionally substituted, monocyclic, C₃₋₇ carbocyclic ring. In some embodiments, R^{44b} and R^{45a} are taken together with their intervening atom to form an optionally substituted, monocyclic, 3- to 14-membered heterocyclic ring. In some embodiments, R^{44b} and R^{45a} are taken together with their intervening atom to form an optionally substituted, monocyclic, 3- to 7-membered heterocyclic ring.

In some embodiments, R^{45a} and R^{45b} are taken together with their intervening atom to form an optionally substituted, monocyclic, carbocyclic or heterocyclic ring. In some embodiments, R^{45a} and R^{45b} are taken together with their intervening atom to form an optionally substituted, monocyclic, C₃₋₁₄ carbocyclic ring. In some embodiments, R^{45a} and R^{45b} are taken together with their intervening atom to form an optionally substituted, monocyclic, C₃₋₇ carbocyclic ring. In some embodiments, R^{45a} and R^{45b} are taken together with their intervening atom to form an optionally substituted, monocyclic, 3- to 14-membered heterocyclic ring. In some embodiments, R^{45a} and R^{45b} are taken together with their intervening atom to form an optionally substituted, monocyclic, 3- to 7-membered heterocyclic ring.

In some embodiments, R^{43b} and R^{46a} are taken together with their intervening atom to form an optionally substituted, monocyclic, carbocyclic or heterocyclic ring. In some embodiments, R^{45b} and R^{46a} are taken together with their intervening atom to form an optionally substituted, monocyclic, C₃₋₁₄ carbocyclic ring. In some embodiments, R^{45b} and R^{46a} are taken together with their intervening atom to form an optionally substituted, monocyclic, C₃₋₇ carbocyclic ring. In some embodiments, R^{45b} and R^{46a} are taken together with their intervening atom to form an optionally substituted, monocyclic, 3- to 14-membered heterocyclic ring. In some embodiments, R^{45b} and R^{46a} are taken together with their intervening atom to form an optionally substituted, monocyclic, 3- to 7-membered heterocyclic ring.

In some embodiments, R^{46a} and R^{46b} are taken together with their intervening atom to form an optionally substituted, monocyclic, carbocyclic or heterocyclic ring. In some embodiments, R^{46a} and R^{46b} are taken together with their intervening atom to form an optionally substituted, monocyclic, C₃₋₁₄ carbocyclic ring. In some embodiments, R^{46a} and R^{46b} are taken together with their intervening atom to form an optionally substituted, monocyclic, C₃₋₇ carbocyclic ring. In some embodiments, R^{46a} and R^{46b} are taken together with their intervening atom to form an optionally substituted, monocyclic, 3- to 14-membered heterocyclic ring. In some embodiments, R^{46a} and R^{46b} are taken together with their intervening atom to form an optionally substituted, monocyclic, 3- to 7-membered heterocyclic ring.

In some embodiments, Formula V-A or V-B is

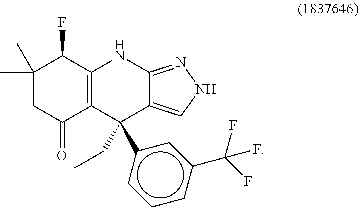

In some embodiments, Formula V-A and V-B is any one of the formulae shown in Table 9:

TABLE 9

| | |
|---|---|
| 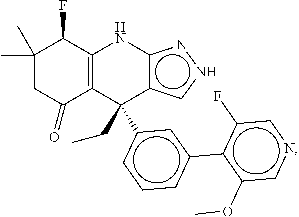 | 948545 |
| 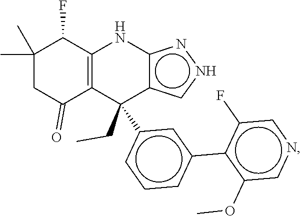 | 948546 |
| 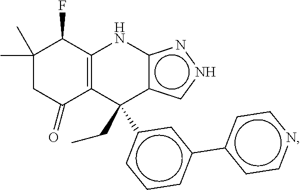 | 959342 |
| 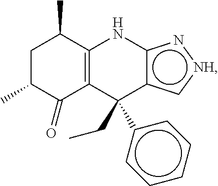 | 865199 |
| 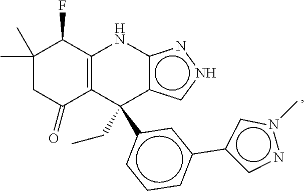 | 957510 |
| 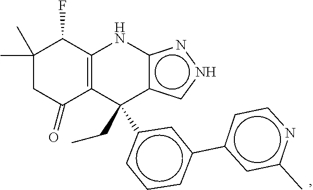 | 946101 |
| 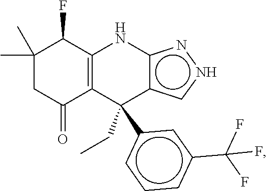 | 837803 |
| 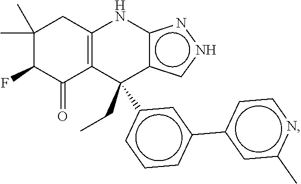 | 945486 |
| 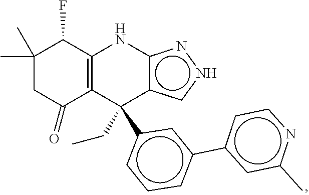 | 946102 |
| 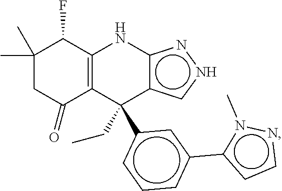 | 957509 |
| 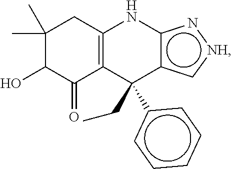 | 946056 |
| 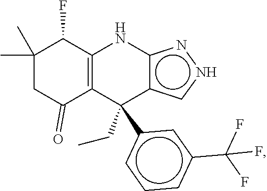 | 837753 |
| 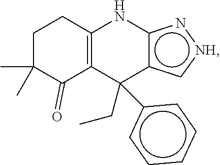 | 835310 |
| 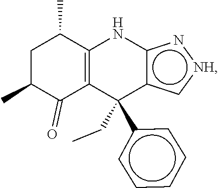 | 865198 |
| 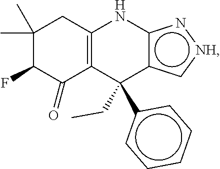 | 957403 |
| 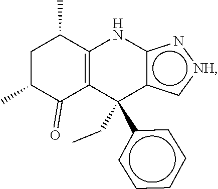 | 909217 |
| 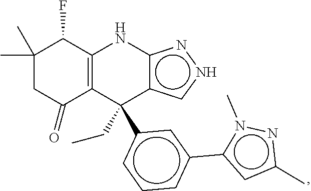 | 947657 |
| 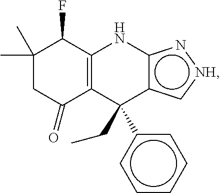 | 957197 |
| 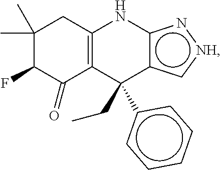 | 957405 |
| 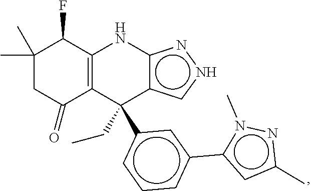 | 947656 |
| 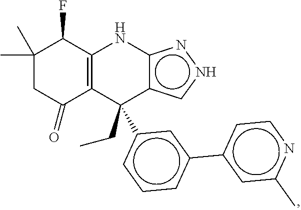 | 946103 |
| 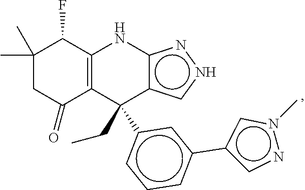 | 957504 |
| 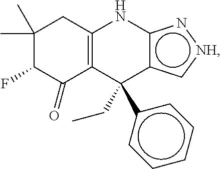 | 826766 |
| 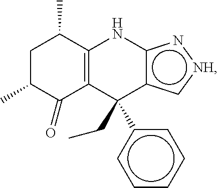 | 909216 |
| 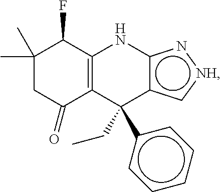 | 957200 |
| 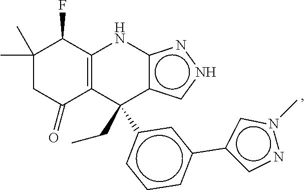 | 957505 |
| 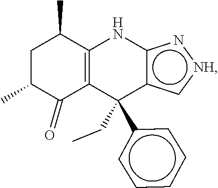 | 865201 |
| 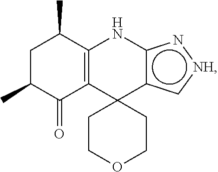 | 893316 |
| 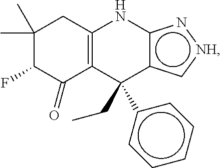 | 826768 |
| 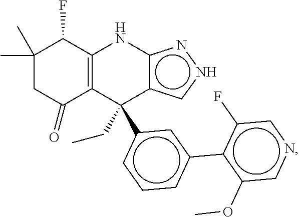 | 948548 |
| 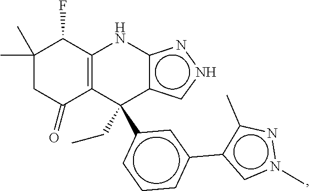 | 947653 |
| 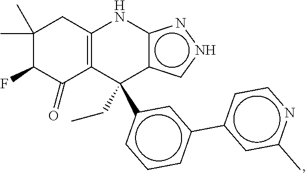 | 945475 |
| 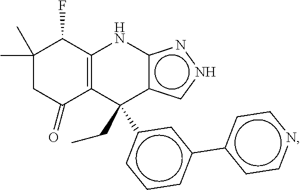 | 959341 |
| 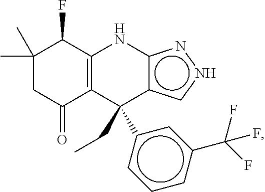 | 837683 |
| 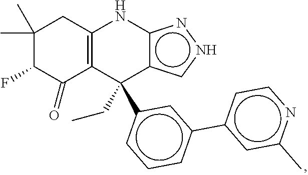 | 945476 |
| 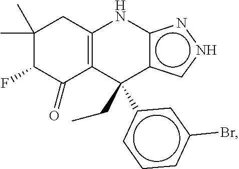 | 835314 |
| 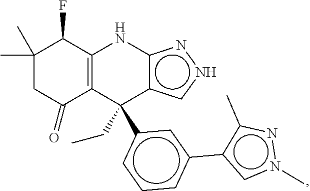 | 947652 |
| 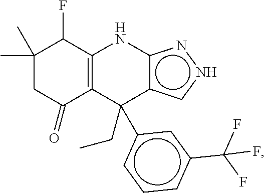 | 819622 |
| 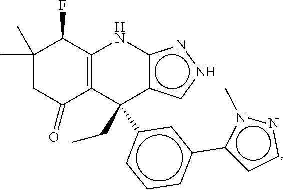 | 957506 |
| 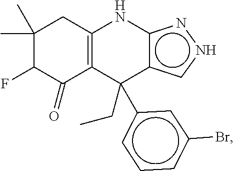 | 826764 |
| 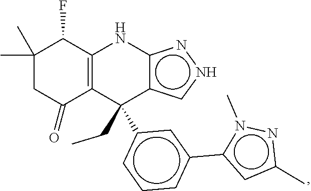 | 947655 |
| 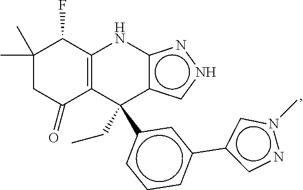 | 957511 |
| 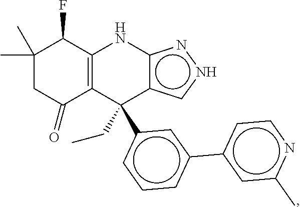 | 946104 |
| 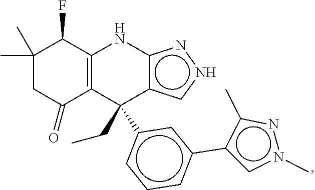 | 947650 |
| 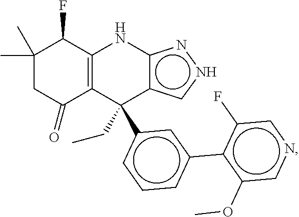 | 948547 |
| 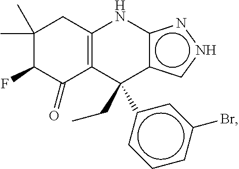 | 835315 |
| 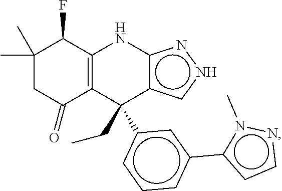 | 957507 |
| 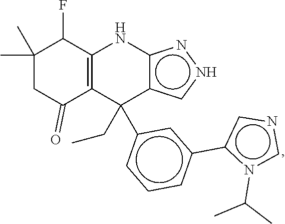 | 963155 |
| 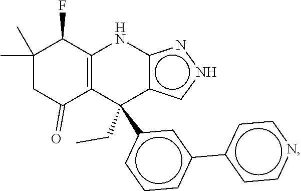 | 959343 |
| 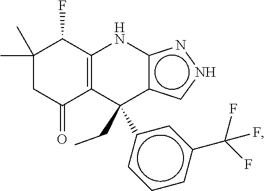 | 837646 |
| 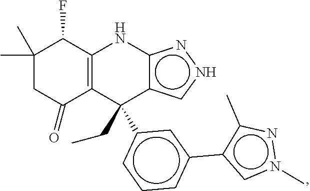 | 947651 |
| 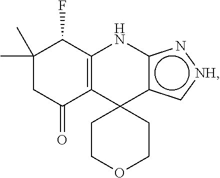 | 883833 |
| 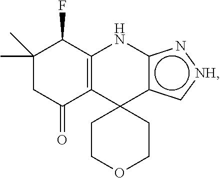 | 883834 |
| 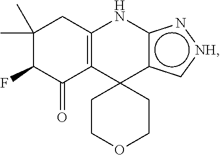 | 883836 |
| 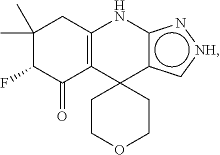 | 883835 |
| 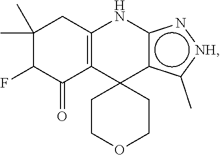 | 912072 |
| 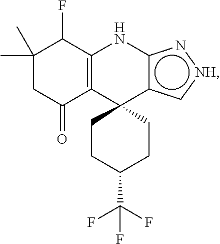 | 917136 |
| 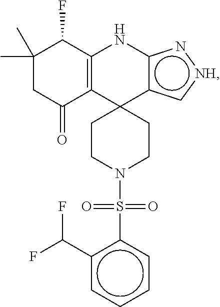 | 951839 |
| 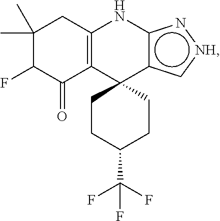 | 917134 |
| 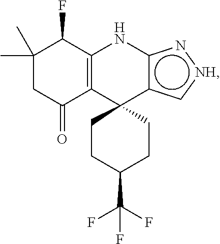 | 918284 |
| 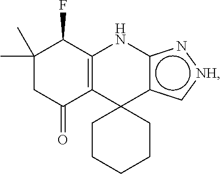 | 951705 |
| 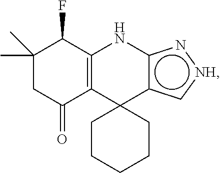 | 951705 |
| 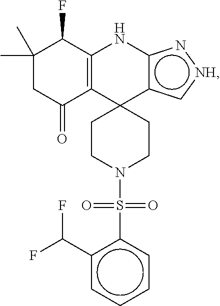 | 951846 |
| 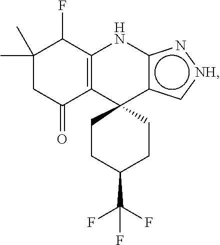 | 917135 |
| 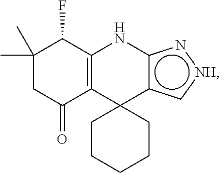 | 951711 |
| 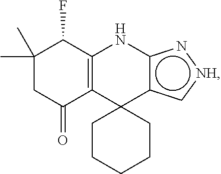 | 951711 |
| 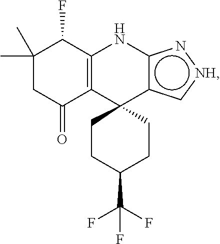 | 918285 |
| 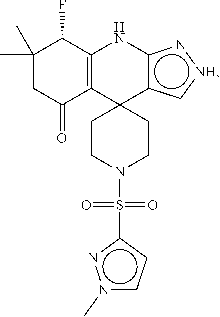 | 951708 |
| 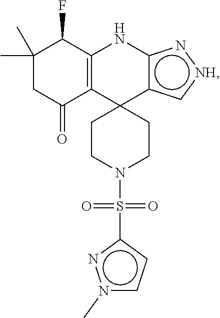 | 951709 |
| 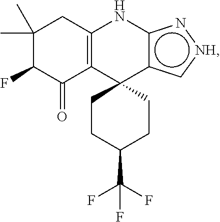 | 918282 |
| 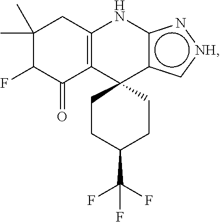 | 917133 |
| 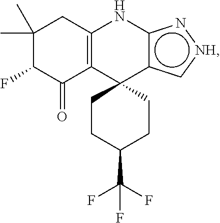 | 918283 |
| 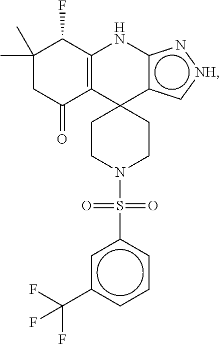 | 951713 |
| 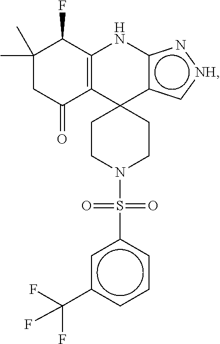 | 951703 |
| 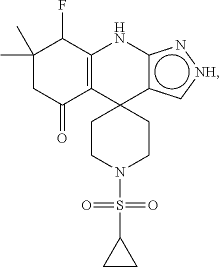 | 950814 |
| 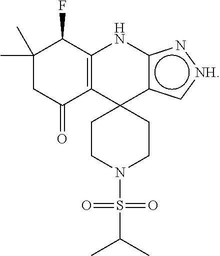 | 951701 |

In some embodiments, Formula V-A and V-B is any one of the formulae shown in Table 10:

TABLE 10

| | |
|---|---|
| 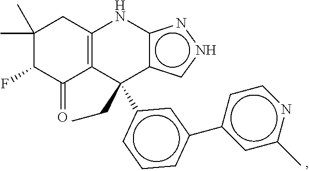 | 945485 |
| 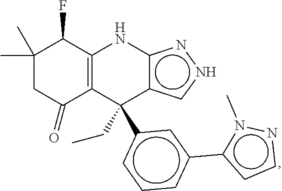 | 957508 |
| 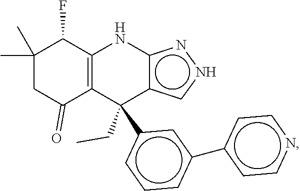 | 959344 |
| 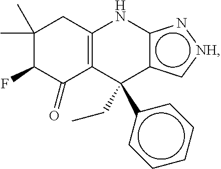 | 826767 |
| 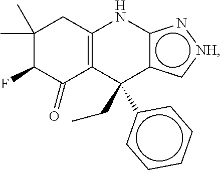 | 826769 |
| 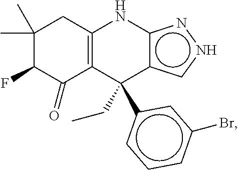 | 835312 |
| 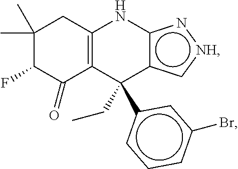 | 835313 |
| 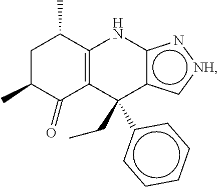 | 865202 |
| 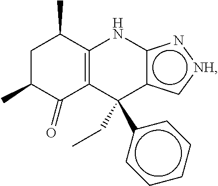 | 909218 |
| 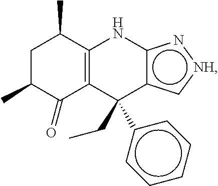 | 909219 |
| 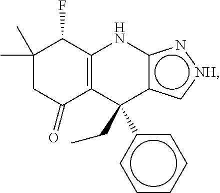 | 957196 |
| 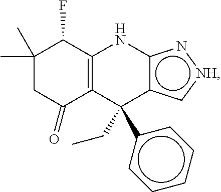 | 957199 |
| 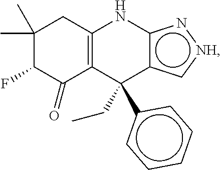 | 957404 |
| 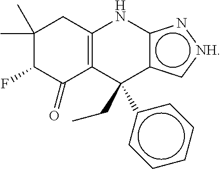 | 957406 |

In some embodiments, Formula V-A and V-B is not any one of the formulae shown in Table 10.

In another aspect, provided herein is a compound, or a pharmaceutically acceptable salt, solvate, hydrate, polymorph, co-crystal, tautomer, stereoisomer, isotopically labeled compound, or prodrug thereof, wherein the compound is of Formula VI-A or VI-B:

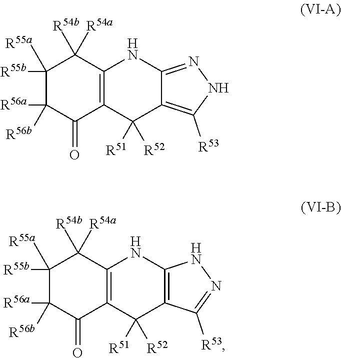

wherein:
R⁵¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R⁵² is optionally substituted alkyl, optionally substituted alkenyl, or optionally substituted alkynyl;
or R³¹ and R⁵² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
R⁵³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{F}, —SCN, —SR^{F}, —SSR^{F}, —N₃, —NO, —N(R^{F})₂, —NO₂, —C(═O)R^{F}, —C(═O)OR^{F}, —C(═O)SR^{F}, —C(═O)N(R^{F})₂, —C(═NR^{F})R^{F}, —C(═NR^{F})OR^{F}, —C(═NR^{F})SR^{F}, —C(═NR^{F})N(R^{F})₂, —S(═O)R^{F}, —S(═O)OR^{F}, —S(═O)SR^{F}, —S(═O)N(R^{F})₂, —S(═O)₂R^{F}, —S(═O)₂OR^{F}, —S(═O)₂SR^{F}, —S(═O)₂N(R^{F})₂, —OC(═O)R^{F}, —OC(═O)OR^{F}, —OC(═O)SR^{F}, —OC(═O)N(R^{F})₂, —OC(═NR^{F})R^{F}, —OC(═NR^{F})OR^{F}, —OC(═NR^{F})SR^{F}, —OC(═NR^{F})N(R^{F})₂, —OS(═O)R^{F}, —OS(═O)OR^{F}, —OS(═O)SR^{F}, —OS(═O)N(R^{F})₂, —OS(═O)₂R^{F}, —OS(═O)₂OR^{F}, —OS(═O)₂SR^{F}, —OS(═O)₂N(R^{F})₂, —ON(R^{F})₂, —SC(═O)R^{F}, —SC(═O)OR^{F}, —SC(═O)SR^{F}, —SC(═O)N(R^{F})₂, —SC(═NR^{F})R^{F}, —SC(═NR^{F})OR^{F}, —SC(═NR^{F})SR^{F}, —SC(═NR^{F})N(R^{F})₂, —NR^{F}C(═O)R^{F}, —NR^{F}C(═O)OR^{F}, —NR^{F}C(═O)SR^{F}, —NR^{F}C(═O)N(R^{F})₂, —NR^{F}C(═NR^{F})R^{F}, —NR^{F}C(═NR^{F})OR^{F}, —NR^{F}C(═NR^{F})SR^{F}, —NR^{F}C(═NR^{F})N(R^{F})₂, —NR^{F}S(═O)R^{F}, —NR^{F}S(═O)OR^{F}, —NR^{F}S(═O)SR^{F}, —NR^{F}S(═O)N(R^{F})₂, —NR^{F}S(═O)₂R^{F}, —NR^{F}S(═O)₂OR^{F}, —NR^{F}S(═O)₂SR^{F}, —NR^{F}S(═O)₂N(R^{F})₂, —Si(R^{F})₃, —Si(R^{F})₂OR^{F}, —Si(R^{F})(OR^{F})₂, —Si(OR^{F})₃, —OSi(R^{F})₃, —Osi(R^{F})₂OR^{F}, —Osi(R^{F})(OR^{F})₂, or —Osi(OR^{F})₃;
each of R^{54a}, R^{54b}, R^{56a}, and R^{56b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each instance of R^{F} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{F} attached to the same intervening atom are joined together with the intervening atom to form an optionally substituted, monocyclic, heterocyclic or heteroaryl ring; and
R^{55a} and R^{55b} are independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{F}, —SCN, —SR^{F}, —SSR^{F}, —N₃, —NO, —N(R^{F})₂, —NO₂—C(═O)R^{F}, —C(═O)OR^{F}, —C(═O)SR^{F}, —C(═O)N(R^{F})₂, —C(═NR^{F})R^{F}, —C(═NR^{F})OR^{F}, —C(═NR^{F})SR^{F}, —C(═NR^{F})N(R^{F})₂, —S(═O)R^{F}, —S(═O)OR^{F}, —S(═O)SR^{F}, —S(═O)N(R^{F})₂, —S(═O)₂R^{F}, —S(═O)₂OR^{F}, —S(═O)₂SR^{F}, —S(═O)₂N(R^{F})₂, —OC(═O)R^{F}, —OC(═O)OR^{F}, —OC(═O)SR^{F}, —OC(═O)N(R^{F})₂, —OC(═NR^{F})R^{F}, —OC(═NR^{F})OR^{F}, —OC(═NR^{F})SR^{F}, —OC(═NR^{F})N(R^{F})₂, —OS(═O)R^{F}, —OS(═O)OR^{F}, —OS(═O)SR^{F}, —OS(═O)N(R^{F})₂, —OS(═O)₂R^{F}, —OS(═O)₂OR^{F}, —OS(═O)₂SR^{F}, —OS(═O)₂N(R^{F})₂, —ON(R^{F})₂, —SC(═O)R^{F}, —SC(═O)OR^{F}, —SC(═O)SR^{F}, —SC(═O)N(R^{F})₂, —SC(═NR^{F})R^{F}, —SC(═NR^{F})OR^{F}, —SC(═NR^{F})SR^{F}, —SC(═NR^{F})N(R^{F})₂, —NR^{F}C(═O)R^{F}, —NR^{F}C(═O)OR^{F}, —NR^{F}C(═O)SR^{F}, —NR^{F}C(═O)N(R^{F})₂, —NR^{F}C(═NR^{F})R^{F}, —NR^{F}C(═NR^{F})OR^{F}, —NR^{F}C(═NR^{F})SR^{F}, —NR^{F}C(═NR^{F})N(R^{F})₂, —NR^{F}S(═O)R^{F}, —NR^{F}S(═O)OR^{F}, —NR^{F}S(═O)SR^{F}, —NR^{F}S(═O)N(R^{F})₂, —NR^{F}S(═O)₂R^{F}, —NR^{F}S(═O)₂OR^{F}, —NR^{F}S(═O)₂SR^{F}, —NR^{F}S(═O)₂N(R^{F})₂, —Si(R^{F})₃, —Si(R^{F})₂OR^{F}, —Si(R^{F})(OR^{F})₂, —Si(OR^{F})₃, —Osi(R^{F})₂, —Osi(R^{E})₂OR^{E}, —Osi(R^{F})(OR^{F})₂, or —Osi(OR^{F})₃;
or R^{54a} and R^{54b} are taken together with their intervening atom to form an optionally substituted, monocyclic, carbocyclic or heterocyclic ring;
or R^{54b} and R^{55a} are taken together with their intervening atom to form an optionally substituted, monocyclic, carbocyclic or heterocyclic ring;
or R^{55a} and R^{55b} are taken together with their intervening atom to form an optionally substituted, monocyclic, 5- to 7-membered heterocyclic ring;
or R^{55b} and R^{56a} are taken together with their intervening atom to form an optionally substituted, monocyclic, carbocyclic or heterocyclic ring;
or R^{56a} and R^{56b} are taken together with their intervening atom to form an optionally substituted, monocyclic, carbocyclic or heterocyclic ring;
provided that at least one of R^{55a} and R^{55b} is not-CH₃.

In some embodiments, the compound is of the formula:

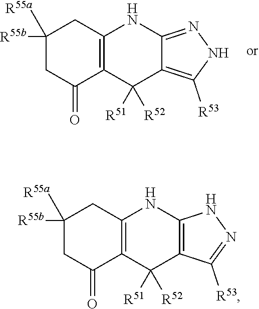

or a pharmaceutically acceptable salt, solvate, hydrate, polymorph, co-crystal, tautomer, stereoisomer, isotopically labeled compound, or prodrug thereof.

In some embodiments, the compound is of the formula:

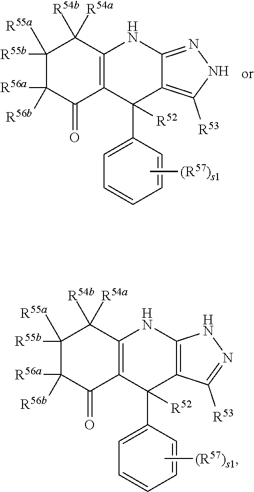

or a pharmaceutically acceptable salt, solvate, hydrate, polymorph, co-crystal, tautomer, stereoisomer, isotopically labeled compound, or prodrug thereof,
wherein:
R^{55a} and R^{55b} are taken together with their intervening atom to form an optionally substituted, monocyclic, 5- to 7-membered heterocyclic ring.

In some embodiments, the compound is of the formula:

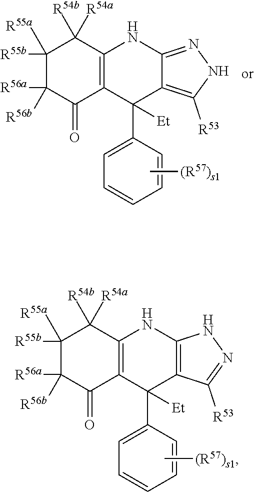

or a pharmaceutically acceptable salt, solvate, hydrate, polymorph, co-crystal, tautomer, stereoisomer, isotopically labeled compound, or prodrug thereof.

In some embodiments,

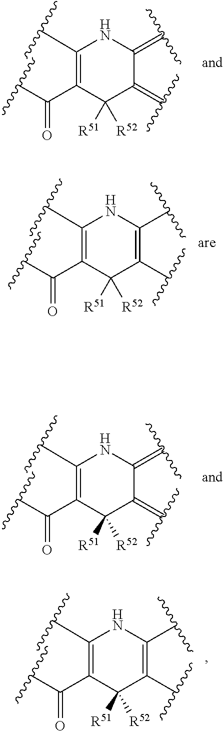

respectively. In some embodiments,

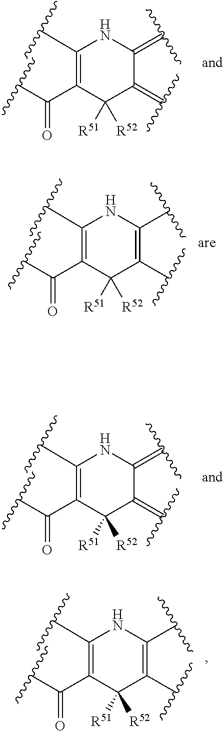

respectively.

In some embodiments, R⁵¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl. In some embodiments, R⁵¹ is optionally substituted C₁₋₁₀ alkyl, optionally substituted C₁₋₁₀ alkenyl, or optionally substituted C₁₋₁₀ alkynyl.

In some embodiments, R⁵¹ is optionally substituted carbocyclyl or optionally substituted heterocyclyl. In some embodiments, R⁵¹ is optionally substituted C₃₋₁₄ carbocyclyl. In some embodiments, R⁵¹ is optionally substituted monocyclic C₃₋₇ carbocyclyl. In some embodiments, R⁵¹ is optionally substituted monocyclic C₃₋₄ carbocyclyl. In some embodiments, R⁵¹ is optionally substituted monocyclic C₅₋₇ carbocyclyl. In some embodiments, R⁵¹ is saturated carbocyclyl. In some embodiments, R⁵¹ is carbocyclyl comprising only one unsaturated bond (e.g., C═C bond) in the carbocyclic ring system.

In some embodiments, R⁵¹ is 3- to 14-membered optionally substituted heterocyclyl. In some embodiments, R⁵¹ is optionally substituted monocyclic 3- to 7-membered heterocyclyl. In some embodiments, R⁵¹ is optionally substituted 3- to 14-membered heterocyclyl comprising one or more O and/or S atoms but no N atoms. In some embodiments, R⁵¹ is optionally substituted 3- to 14-membered heterocyclyl comprising one or more N atoms and optionally one or more O and/S atoms. In some embodiments, R⁵¹ is optionally substituted monocyclic 3- to 7-membered heterocyclyl comprising one or more O and/or S atoms but no N atoms. In some embodiments, R⁵¹ is optionally substituted monocyclic 3- to 7-membered heterocyclyl comprising one or more N atoms and optionally one or more O and/or S atoms. In some embodiments, R⁵¹ is saturated heterocyclyl. In some embodiments, R⁵¹ is heterocyclyl comprising only one unsaturated bond (e.g., C═C bond) in the heterocyclic ring system.

In some embodiments, R⁵¹ is optionally substituted aryl. In some embodiments, R⁵¹ is optionally substituted monocyclic aryl. In some embodiments, R⁵¹ is optionally substituted bicyclic aryl. In some embodiments, R⁵¹ is optionally substituted C₆₋₁₄ aryl. In some embodiments, R⁵¹ is optionally substituted C₆₋₁₀ aryl. In some embodiments, R⁵¹ is optionally substituted phenyl. In some embodiments, R⁵¹ is optionally substituted naphthyl.

In some embodiments, R⁵¹ is optionally substituted heteroaryl. In some embodiments, R⁵¹ is optionally substituted monocyclic heteroaryl. In some embodiments, R⁵¹ is optionally substituted bicyclic heteroaryl. In some embodiments, R⁵¹ is optionally substituted 5- to 14-membered heteroaryl. In some embodiments, R⁵¹ is optionally substituted 5- to 10-membered heteroaryl. In some embodiments, R⁵¹ is optionally substituted 5- to 6-membered monocyclic heteroaryl. In some embodiments, R⁵¹ is optionally substituted 9- to 10-membered bicyclic heteroaryl.

In some embodiments, R⁵¹ is optionally substituted aryl fused with optionally substituted monocyclic C₃₋₇ carbocyclyl. In some embodiments, R⁵¹ is optionally substituted C₆₋₁₄ aryl fused with optionally substituted monocyclic C₃₋₇ carbocyclyl. In some embodiments, R⁵¹ is optionally substituted C₆₋₁₀ aryl fused with optionally substituted monocyclic C₃₋₇ carbocyclyl. In some embodiments, R⁵¹ is optionally substituted phenyl fused with optionally substituted monocyclic C₃₋₇ carbocyclyl. In some embodiments, R⁵¹ is optionally substituted naphthyl fused with optionally substituted monocyclic C₃₋₇ carbocyclyl.

In some embodiments, R⁵¹ is optionally substituted aryl fused with optionally substituted monocyclic 3- to 7-membered heterocyclyl. In some embodiments, R⁵¹ is optionally substituted C₆₋₁₄ aryl fused with optionally substituted monocyclic 3- to 7-membered heterocyclyl. In some embodiments, R⁵¹ is optionally substituted C₆₋₁₀ aryl fused with optionally substituted monocyclic 3- to 7-membered heterocyclyl. In some embodiments, R⁵¹ is optionally substituted phenyl fused with optionally substituted monocyclic 3- to 7-membered heterocyclyl. In some embodiments, R⁵¹ is optionally substituted naphthyl fused with optionally substituted monocyclic 3- to 7-membered heterocyclyl.

In some embodiments, R⁵¹ is optionally substituted heteroaryl fused with optionally substituted monocyclic C₃₋₇ carbocyclyl. In some embodiments, R⁵¹ is optionally substituted 5- to 14-membered heteroaryl fused with optionally substituted monocyclic C₃₋₇ carbocyclyl. In some embodiments, R⁵¹ is optionally substituted 5- to 10-membered heteroaryl fused with optionally substituted monocyclic C₃₋₇ carbocyclyl. In some embodiments, R⁵¹ is optionally substituted 5- to 6-membered monocyclic heteroaryl fused with optionally substituted monocyclic C₃₋₇ carbocyclyl. In some embodiments, R⁵¹ is optionally substituted 9- to 10-membered bicyclic heteroaryl fused with optionally substituted monocyclic C₃₋₇ carbocyclyl.

In some embodiments, R⁵¹ is optionally substituted heteroaryl fused with optionally substituted monocyclic 3- to 7-membered heterocyclyl. In some embodiments, R⁵¹ is optionally substituted 5- to 14-membered heteroaryl fused with optionally substituted monocyclic 3- to 7-membered heterocyclyl. In some embodiments, R⁵¹ is optionally substituted 5- to 10-membered heteroaryl fused with optionally substituted monocyclic 3- to 7-membered heterocyclyl. In some embodiments, R⁵¹ is optionally substituted 5- to 6-membered monocyclic heteroaryl fused with optionally substituted monocyclic 3- to 7-membered heterocyclyl. In some embodiments, R⁵¹ is optionally substituted 9- to 10-membered bicyclic heteroaryl fused with optionally substituted monocyclic 3- to 7-membered heterocyclyl.

In some embodiments, R⁵¹ is

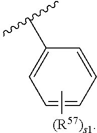

Each instance of R⁵⁷ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)R^{D}), —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃.

s1 is 0, 1, 2, 3, 4, or 5. In some embodiments, s1 is 0, 1, 2, 3, or 4. In some embodiments, s1 is 0, 1, 2, or 3. In some embodiments, s1 is 0, 1, or 2. In some embodiments, s1 is 0 or 1. In some embodiments, s1 is 0. In some embodiments, s1 is 1. In some embodiments, s1 is 2. In some embodiments, s1 is 3. In some embodiments, s1 is 4. In some embodiments, s1 is 5.

In some embodiments, R⁵¹ is

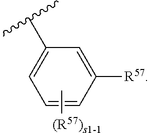

In some embodiments, R⁵¹ is

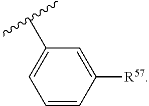

In some embodiments, R⁵¹ is

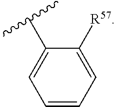

In some embodiments, R⁵¹ is

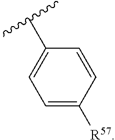

In some embodiments, R⁵¹ is

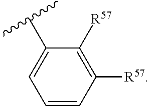

In some embodiments, R⁵¹ is

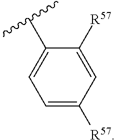

In some embodiments, R⁵¹ is

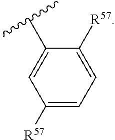

In some embodiments, R⁵¹ is

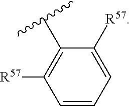

In some embodiments, R⁵¹ is

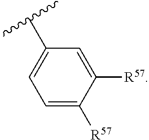

In some embodiments, R⁵¹ is

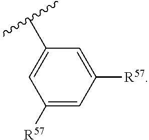

In some embodiments, at least one instance of R⁵⁷ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{D}, —SR^{D}, or —N(R^{D})₂. In some embodiments, at least one instance of R⁵⁷ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, at least one instance of R⁵⁷ is C₁₋₁₀ haloalkyl. In some embodiments, at least one instance of R⁵⁷ is C₁₋₄ haloalkyl. In some embodiments, at least one instance of R⁵⁷ is C₁₋₄ fluoroalkyl (e.g., C₁₋₄ perfluoroalkyl). In some embodiments, at least one instance of R⁵⁷ is —CF₃.

In some embodiments, at least one instance of R⁵⁷ is halogen. In some embodiments, at least one instance of R⁵⁷ is bromine, chlorine, or fluorine. In some embodiments, at least one instance of R⁵⁷ is bromine or chlorine. In some embodiments, at least one instance of R⁵⁷ is chlorine or fluorine. In some embodiments, at least one instance of R⁵⁷ is bromine. In some embodiments, at least one instance of R⁵⁷ is chlorine. In some embodiments, at least one instance of R⁵⁷ is fluorine.

In some embodiments, at least one instance of R⁵⁷ is —OR^{D}, —SR^{D}, or —N(R^{D})₂ (e.g., wherein R^{D} is hydrogen or optionally substituted alkyl, or optionally substituted phenyl). In some embodiments, at least one instance of R⁵⁷ is —OH. In some embodiments, at least one instance of R⁵⁷ is —SH. In some embodiments, at least one instance of R⁵⁷ is —NH₂. In some embodiments, at least one instance of R⁵⁷ is —CN, —SCN, —SSR^{D}, —N₃, —NO, or —NO₂. In some embodiments, at least one instance of R⁵⁷ is —C(═O)R^{D}, —C(═O)OR^{D}, —C(═O)SR^{D}, or —C(═O)N(R^{D})₂ (e.g., wherein R^{D} is hydrogen or optionally substituted alkyl, or optionally substituted phenyl). In some embodiments, at least one instance of R⁵⁷ is —C(═NR^{D})R^{D}, —C(═NR^{D})OR^{D}, —C(═NR^{D})SR^{D}, or —C(═NR^{D})N(R^{D})₂ (e.g., wherein R^{D} is hydrogen or optionally substituted alkyl, or optionally substituted phenyl). In some embodiments, at least one instance of R⁵⁷ is —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, or —S(═O)₂N(R^{D})₂ (e.g., wherein R^{D} is hydrogen or optionally substituted alkyl, or optionally substituted phenyl). In some embodiments, at least one instance of R⁵⁷ is —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)RP, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, or —ON(R^{D})₂ (e.g., wherein R^{D} is hydrogen or optionally substituted alkyl, or optionally substituted phenyl). In some embodiments, at least one instance of R⁵⁷ is —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, or —SC(═NR^{D})N(R^{D})₂ (e.g., wherein R^{D} is hydrogen or optionally substituted alkyl, or optionally substituted phenyl). In some embodiments, at least one instance of R⁵⁷ is —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, or —NR^{D}S(═O)₂N(R^{D})₂ (e.g., wherein R^{D} is hydrogen or optionally substituted alkyl, or optionally substituted phenyl). In some embodiments, at least one instance of R⁵⁷ is —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃ (e.g., wherein R^{D} is hydrogen or optionally substituted alkyl, or optionally substituted phenyl).

In some embodiments, at least one instance of R⁵⁷ is optionally substituted carbocyclyl, optionally substituted heterocyclyl, optionally substituted aryl, or optionally substituted heteroaryl. In some embodiments, at least one instance of R⁵⁷ is optionally substituted C₃₋₁₄ carbocyclyl. In some embodiments, at least one instance of R⁵⁷ is optionally substituted monocyclic C₃₋₇ carbocyclyl. In some embodiments, at least one instance of R⁵⁷ is optionally substituted monocyclic C₃₋₄ carbocyclyl. In some embodiments, at least one instance of R⁵⁷ is optionally substituted monocyclic C₅₋₇ carbocyclyl. In some embodiments, at least one instance of R⁵⁷ is saturated carbocyclyl. In some embodiments, at least one instance of R⁵⁷ is carbocyclyl comprising only one unsaturated bond (e.g., C═C bond) in the carbocyclic ring system.

In some embodiments, at least one instance of R⁵⁷ is optionally substituted 3- to 14-membered heterocyclyl. In some embodiments, at least one instance of R⁵⁷ is optionally substituted monocyclic 3- to 7-membered heterocyclyl. In some embodiments, at least one instance of R⁵⁷ is optionally substituted 3- to 14-membered heterocyclyl comprising one or more O and/or S atoms but no N atoms. In some embodiments, at least one instance of R⁵⁷ is optionally substituted 3- to 14-membered heterocyclyl comprising one or more N atoms and optionally one or more O and/S atoms. In some embodiments, at least one instance of R⁵⁷ is optionally substituted monocyclic 3- to 7-membered heterocyclyl comprising one or more O and/or S atoms but no N atoms. In some embodiments, at least one instance of R⁵⁷ is optionally substituted monocyclic 3- to 7-membered heterocyclyl comprising one or more N atoms and optionally one or more O and/or S atoms. In some embodiments, at least one instance of R⁵⁷ is saturated heterocyclyl. In some embodiments, at least one instance of R⁵⁷ is heterocyclyl comprising only one unsaturated bond (e.g., C═C bond) in the heterocyclic ring system.

In some embodiments, at least one instance of R⁵⁷ is optionally substituted monocyclic aryl. In some embodiments, at least one instance of R⁵⁷ is optionally substituted bicyclic aryl. In some embodiments, at least one instance of R⁵⁷ is optionally substituted C₆₋₁₄ aryl. In some embodiments, at least one instance of R⁵⁷ is optionally substituted C₆₋₁₀ aryl. In some embodiments, at least one instance of R⁵⁷ is optionally substituted phenyl. In some embodiments, at least one instance of R⁵⁷ is optionally substituted naphthyl.

In some embodiments, at least one instance of R⁵⁷ is optionally substituted monocyclic heteroaryl. In some embodiments, at least one instance of R⁵⁷ is optionally substituted bicyclic heteroaryl. In some embodiments, at least one instance of R⁵⁷ is optionally substituted 5- to 14-membered heteroaryl. In some embodiments, at least one instance of R⁵⁷ is optionally substituted 5- to 10-membered heteroaryl. In some embodiments, at least one instance of R⁵⁷ is optionally substituted 5- to 6-membered monocyclic heteroaryl. In some embodiments, at least one instance of R⁵⁷ is optionally substituted heteroaryl comprising one or more N atoms. In some embodiments, at least one instance of R⁵⁷ is optionally substituted pyridinyl or optionally substituted pyrazolyl.

In some embodiments, at least one instance of R^{D} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{D} is optionally substituted carbocyclyl, optionally substituted heterocyclyl, optionally substituted aryl, or optionally substituted heteroaryl. In some embodiments, at least one instance of R^{D} is independently hydrogen, optionally substituted C₁₋₁₀ alkyl, optionally substituted Cujo alkenyl, optionally substituted C₁₋₁₀ alkynyl, optionally substituted C₃₋₁₄ carbocyclyl, or optionally substituted C₆₋₁₄ aryl. In some embodiments, at least one instance of RP is independently hydrogen, optionally substituted C₁₋₁₀ alkyl, or optionally substituted phenyl. In some embodiments, at least one instance of R^{D} is a nitrogen protecting group when attached to a nitrogen atom, an oxygen protecting group when attached to an oxygen atom, or a sulfur protecting group when attached to a sulfur atom. In some embodiments, two instances of R^{D} attached to the same intervening atom are joined together with the intervening atom to form an optionally substituted, monocyclic, heterocyclic or heteroaryl ring.

In some embodiments, R⁵² is optionally substituted alkyl, optionally substituted alkenyl, or optionally substituted alkynyl. In some embodiments, R⁵² is optionally substituted C₁₋₄ alkyl, optionally substituted C₁₋₄ alkenyl, or optionally substituted C₁₋₄ alkynyl. In some embodiments, R⁵² is optionally substituted alkyl. In some embodiments, R⁵² is optionally substituted C₁₋₄ alkyl. In some embodiments, R⁵² is unsubstituted C₁-C₄ alkyl. In some embodiments, R⁵² is unsubstituted methyl. In some embodiments, R⁵² is unsubstituted ethyl.

In some embodiments, R⁵¹ and R⁵² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring. In some embodiments, R⁵¹ and R⁵² are taken together with their intervening atom to form an optionally substituted, monocyclic, carbocyclic ring. In some embodiments, R⁵¹ and R⁵² are taken together with their intervening atom to form an optionally substituted, monocyclic, 3- to 7-membered carbocyclic ring. In some embodiments, R⁵¹ and R⁵² are taken together with their intervening atom to form an optionally substituted, monocyclic, 5- to 6-membered carbocyclic ring. In some embodiments, R⁵¹ and R⁵² are taken together with their intervening atom to form an optionally substituted, monocyclic, heterocyclic ring. In some embodiments, R⁵¹ and R⁵² are taken together with their intervening atom to form an optionally substituted, monocyclic, 3- to 7-membered heterocyclic ring. In some embodiments, R⁵¹ and R⁵² are taken together with their intervening atom to form an optionally substituted, monocyclic, 5- to 6-membered heterocyclic ring. In some embodiments, R⁵¹ and R⁵² are taken together with their intervening atom to form an optionally substituted, monocyclic, heterocyclic ring comprising one or more N atoms. In some embodiments, R⁵¹ and R⁵² are taken together with their intervening atom to form an optionally substituted, monocyclic, 3- to 7-membered heterocyclic ring comprising one or more N atoms. In some embodiments, R⁵¹ and R⁵² are taken together with their intervening atom to form an optionally substituted tetrahydropyranyl.

In some embodiments, R⁵³ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{F}, —SR^{F}, or —N(R^{F})₂. In some embodiments, R⁵³ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R⁵³ is C₁₋₁₀ haloalkyl. In some embodiments, R⁵³ is C₁₋₄ haloalkyl. In some embodiments, R⁵³ is C₁₋₄ fluoroalkyl (e.g., C₁₋₄ perfluoroalkyl). In some embodiments, R⁵³ is —CF₃. In some embodiments, R⁵³ is hydrogen, optionally substituted C₁-C₆ alkyl, or halogen. In some embodiments, R⁵³ is hydrogen, fluorine, —CH₃, —CH₂F, —CHF₂, or —CF₃. In some embodiments, R⁵³ is hydrogen, fluorine, —CH₃, or —CF₃. In some embodiments, R⁵³ is hydrogen or halogen. In some embodiments, R⁵³ is hydrogen or fluorine. In some embodiments, R⁵³ is hydrogen or optionally substituted C₁-C₆ alkyl. In some embodiments, R⁵³ is hydrogen, unsubstituted C₁-C₆ alkyl, or C₁₋₆ haloalkyl. In some embodiments, R⁵³ is hydrogen.

In some embodiments, R⁵³ is halogen. In some embodiments, R⁵³ is bromine, chlorine, or fluorine. In some embodiments, R⁵³ is bromine or chlorine. In some embodiments, R⁵³ is chlorine or fluorine. In some embodiments, R⁵³ is bromine. In some embodiments, R⁵³ is chlorine. In some embodiments, R⁵³ is fluorine.

In some embodiments, R⁵³ is —OR^{F}, —SR^{E}, or —N(R^{E})₂ (e.g., wherein R^{F} is hydrogen or optionally substituted alkyl, or optionally substituted phenyl). In some embodiments, R⁵³ is —OH. In some embodiments, R⁵³ is —SH. In some embodiments, R⁵³ is —NH₂. In some embodiments, R⁵³ is —CN, —SCN, —SSR^{F}, —N₃, —NO, or —NO₂. In some embodiments, R⁵³ is —C(═O)R^{F}, —C(═O)OR^{F}, —C(═O)SR^{F}, or —C(═O)N(R^{F})₂ (e.g., wherein R^{F} is hydrogen or optionally substituted alkyl, or optionally substituted phenyl). In some embodiments, R⁵³ is —C(═NR^{F})R^{F}, —C(═NR^{F})OR^{F}, —C(═NR^{F})SR^{F}, or —C(═NR^{F})N(R^{E})₂ (e.g., wherein R^{F} is hydrogen or optionally substituted alkyl, or optionally substituted phenyl). In some embodiments, R⁵³ is —S(═O)R^{F}, —S(═O)OR^{F}, —S(═O)SR^{F}, —S(═O)N(R^{F})₂, —S(═O)₂R^{F}, —S(═O)₂OR^{F}, —S(═O)₂SR^{F}, or —S(═O)₂N(R^{F})₂ (e.g., wherein R^{F} is hydrogen or optionally substituted alkyl, or optionally substituted phenyl). In some embodiments, R⁵³ is —OC(═O)R^{F}, —OC(═O)OR^{F}, —OC(═O)SR^{F}, —OC(═O)N(R^{F})₂, —OC(═NR^{F})R^{F}, —OC(═NR^{F})OR^{F}, —OC(═NR^{F})SR^{F}, —OC(═NR^{F})N(R^{F})₂, —OS(═O)R^{F}, —OS(═O)OR^{F}, —OS(═O)SR^{F}, —OS(═O)N(R^{F})₂, —OS(═O)₂R^{F}, —OS(═O)₂OR^{F}, —OS(═O)₂SR^{F}, —OS(═O)₂N(R^{F})₂, or —ON(R^{F})₂ (e.g., wherein R^{F} is hydrogen or optionally substituted alkyl, or optionally substituted phenyl). In some embodiments, R⁵³ is —SC(═O)R^{F}, —SC(═O)OR^{F}, —SC(═O)SR^{F}, —SC(═O)N(R^{F})₂, —SC(═NR^{F})R^{F}, —SC(═NR^{F})OR^{F}, —SC(═NR^{F})SR^{F}, or —SC(═NR^{F})N(R^{F})₂ (e.g., wherein R^{F} is hydrogen or optionally substituted alkyl, or optionally substituted phenyl). In some embodiments, R⁵³ is —NR^{F}C(═O)R^{F}, —NR^{F}C(═O)OR^{F}, —NR^{F}C(═O)SR^{F}, —NR^{F}C(═O)N(R^{F})₂, —NR^{F}C(═NR^{F})R^{F}, —NR^{F}C(═NR^{F})OR^{F}, —NR^{F}C(═NR^{F})SR^{F}, —NR^{F}C(═NR^{F})N(R^{F})₂, —NR^{F}S(═O)R^{F}, —NR^{F}S(═O)OR^{F}, —NR^{F}S(═O)SR^{F}, —NR^{F}S(═O)N(R^{F})₂, —NR^{F}S(═O)₂R^{F}, —NR^{F}S(═O)₂OR^{F}, —NR^{F}S(═O)₂SR^{F}, or —NR^{F}S(═O)₂N(R^{F})₂ (e.g., wherein R^{F} is hydrogen or optionally substituted alkyl, or optionally substituted phenyl). In some embodiments, R⁵³ is —Si(R^{F})₃, —Si(R^{F})₂OR^{F}, —Si(R^{F})(OR^{F})₂, —Si(OR^{F})₃, —OSi(R^{F})₃, —OSi(R^{F})₂OR^{F}, —OSi(R^{F})(OR^{F})₂, or —OSi(OR^{F})₃ (e.g., wherein R^{F} is hydrogen or optionally substituted alkyl, or optionally substituted phenyl).

In some embodiments, R⁵³ is optionally substituted carbocyclyl, optionally substituted heterocyclyl, optionally substituted aryl, or optionally substituted heteroaryl. In some embodiments. R⁵³ is optionally substituted C₃₋₁₄ carbocyclyl. In some embodiments, R⁵³ is optionally substituted monocyclic C₃₋₇ carbocyclyl. In some embodiments, R⁵³ is optionally substituted monocyclic C₃₋₄ carbocyclyl. In some embodiments, R⁵³ is optionally substituted monocyclic C₅₋₇ carbocyclyl. In some embodiments, R⁵³ is saturated carbocyclyl. In some embodiments, R⁵³ is carbocyclyl comprising only one unsaturated bond (e.g., C═C bond) in the carbocyclic ring system.

In some embodiments, R⁵³ is optionally substituted 3- to 14-membered heterocyclyl. In some embodiments, R⁵³ is optionally substituted monocyclic 3- to 7-membered heterocyclyl. In some embodiments, R⁵³ is optionally substituted 3- to 14-membered heterocyclyl comprising one or more O and/or S atoms but no N atoms. In some embodiments, R⁵³ is optionally substituted 3- to 14-membered heterocyclyl comprising one or more N atoms and optionally one or more O and/S atoms. In some embodiments, R⁵³ is optionally substituted monocyclic 3- to 7-membered heterocyclyl comprising one or more O and/or S atoms but no N atoms. In some embodiments, R⁵³ is optionally substituted monocyclic 3- to 7-membered heterocyclyl comprising one or more N atoms and optionally one or more O and/or S atoms. In some embodiments, R⁵³ is saturated heterocyclyl. In some embodiments, R⁵³ is heterocyclyl comprising only one unsaturated bond (e.g., C═C bond) in the heterocyclic ring system.

In some embodiments, R³³ is optionally substituted monocyclic aryl. In some embodiments, R⁵³ is optionally substituted bicyclic aryl. In some embodiments, R⁵³ is optionally substituted C₆₋₁₄ aryl. In some embodiments, R⁵³ is optionally substituted C₆₋₁₀ aryl. In some embodiments, R⁵³ is optionally substituted phenyl. In some embodiments, R⁵³ is optionally substituted naphthyl.

In some embodiments, R⁵³ is optionally substituted monocyclic heteroaryl. In some embodiments, R⁵³ is optionally substituted bicyclic heteroaryl. In some embodiments, R⁵³ is optionally substituted 5- to 14-membered heteroaryl. In some embodiments, R⁵³ is optionally substituted 5- to 10-membered heteroaryl. In some embodiments, R⁵³ is optionally substituted 5- to 6-membered monocyclic heteroaryl.

In some embodiments, at least one instance of R^{F} is hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{F} is optionally substituted carbocyclyl, optionally substituted heterocyclyl, optionally substituted aryl, or optionally substituted heteroaryl. In some embodiments, at least one instance of R^{F} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{F} is independently hydrogen, optionally substituted C₁₋₁₀ alkyl, or optionally substituted phenyl. In some embodiments, at least one instance of R^{F} is a nitrogen protecting group when attached to a nitrogen atom, an oxygen protecting group when attached to an oxygen atom, or a sulfur protecting group when attached to a sulfur atom. In some embodiments, two instances of R^{F} attached to the same intervening atom are joined together with the intervening atom to form an optionally substituted, monocyclic, heterocyclic or heteroaryl ring.

In some embodiments, each of R^{54a}, R^{54b}, R^{56a}, and R^{56b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, each of R^{54a}, R^{54b}, R^{56a}, and R^{56b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, each of R^{54a}, R^{54b}, R^{56a}, and R^{56b} is independently hydrogen, halogen, or optionally substituted alkyl. In some embodiments, each of R^{54a}, R^{54b}, R^{56a}, and R^{56b} is independently hydrogen, halogen, or optionally substituted C₁₋₄ alkyl. In some embodiments, each of R^{54a}, R^{54b}, R^{56a}, and R^{56b} is independently hydrogen, fluorine, or optionally substituted C₁₋₄ alkyl. In some embodiments, each of R^{54a}, R^{54b}, R^{56a}, and R^{56b} is independently hydrogen or halogen. In some embodiments, each of R^{54a}, R^{54b}, R^{56a}, and R^{56b} is independently hydrogen or fluorine. In some embodiments, each of R^{54a}, R^{54b}, R^{56a}, and R^{56b} is independently hydrogen or optionally substituted C₁₋₄ alkyl. In some embodiments, at least one of R^{54a}, R^{54b}, R^{56a}, and R^{56b} is hydrogen. In some embodiments, each of R^{54a}, R^{54b}, R^{56a}, and R^{56b} is hydrogen. In some embodiments, at least one of R^{54a}, R^{54b}, R^{56a}, and R^{56b} is optionally substituted C₁₋₄ alkyl. In some embodiments, at least one of R^{54a}, R^{54b}, R^{56a}, and R^{56b} is unsubstituted methyl. In some embodiments, at least one of R^{54a}, R^{54b}, R^{56a}, and R^{56b} is fluorine.

In some embodiments, each of R^{54a} and R^{54b} is independently hydrogen, halogen, or optionally substituted alkyl. In some embodiments, at least one of R^{54a} and R⁵⁴⁵ is hydrogen. In some embodiments, one of R^{54a} and R^{54b} is hydrogen. In some embodiments, R^{54a} and R^{54b} are hydrogen. In some embodiments, at least one of R^{54a} and R^{54b} is optionally substituted C₁₋₄ alkyl. In some embodiments, one of R^{54a} and R^{54b} is optionally substituted C₁₋₄ alkyl. In some embodiments, R^{54a} and R^{54b} are optionally substituted C₁₋₄ alkyl. In some embodiments, at least one of R^{54a} and R^{54b} is —CH₃. In some embodiments, one of R^{54a} and R^{54b} is —CH₃. In some embodiments, one of R^{54a} and R^{54b} are —CH₃.

In some embodiments, each of R^{56a} and R^{56b} is independently hydrogen, halogen, or optionally substituted alkyl. In some embodiments, at least one of R^{56a} and R^{56b} is hydrogen. In some embodiments, one of R^{56a} and R^{56b} is hydrogen. In some embodiments, R^{56a} and R^{56b} are hydrogen. In some embodiments, at least one of R^{56a} and R^{56b} is optionally substituted C₁₋₄ alkyl. In some embodiments, one of R^{56a} and R^{56b} is optionally substituted C₁₋₄ alkyl. In some embodiments, R^{56a} and R^{56b} are optionally substituted C₁₋₄ alkyl. In some embodiments, at least one of R^{56a} and R^{56b} is —CH₃. In some embodiments, one of R^{56a} and R^{56b} is —CH₃. In some embodiments, R^{56a} and R^{56b} are —CH₃.

In some embodiments,

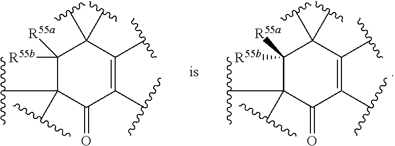

In some embodiments,

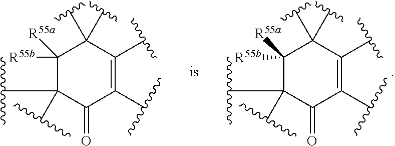

In some embodiments, R^{55a} is not-CH₃. In some embodiments, R^{55b} is not-CH₃. In some embodiments, R^{55a} and R^{55b} are not-CH₃.

In some embodiments, R^{55a} and R^{55b} are independently hydrogen, halogen, optionally substituted alkyl, optionally substituted heteroalkyl, or optionally substituted aryl. In some embodiments, one of R^{55a} and R^{55b} is hydrogen. In some embodiments, R^{55a} and R^{55b} are both hydrogen.

In some embodiments, R^{55a} is halogen, optionally substituted alkyl, optionally substituted alkenyl, optionally substituted alkynyl, optionally substituted heteroalkyl, optionally substituted heteroalkenyl, optionally substituted heteroalkynyl, —OR^{F}, —SR^{F}, or —N(R^{F})₂. In some embodiments, R^{55a} is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R^{55a} is C₁₋₁₀ haloalkyl. In some embodiments, R^{55a} is C₁₋₄ haloalkyl. In some embodiments, R^{55a} is C₁₋₄ fluoroalkyl (e.g., C₁₋₄ perfluoroalkyl). In some embodiments, R^{55a} is —CF₃. In some embodiments, R^{55a} is hydrogen, optionally substituted C₁-C₆ alkyl, or halogen. In some embodiments, R^{55a} is hydrogen, fluorine, —CH₃, —CH₂F, —CHF₂, or —CF₃. In some embodiments, R^{55a} is hydrogen, fluorine, —CH₃, or —CF₃. In some embodiments, R^{55a} is hydrogen or halogen. In some embodiments, R^{35a} is hydrogen or fluorine. In some embodiments, R^{55a} is hydrogen or optionally substituted C₁-C₆ alkyl. In some embodiments, R^{55a} is hydrogen, unsubstituted C₁-C₆ alkyl, or C₁₋₆ haloalkyl. In some embodiments, R^{55a} is hydrogen.

In some embodiments, R^{55a} is halogen. In some embodiments, R^{55a} is bromine, chlorine, or fluorine. In some embodiments, R^{55a} is bromine or chlorine. In some embodiments, R^{55a} is chlorine or fluorine. In some embodiments, R^{55a} is bromine. In some embodiments, R^{55a} is chlorine. In some embodiments, R^{55a} is fluorine.

In some embodiments, R^{55a} is —OR^{F}, —SR^{F}, or —N(R^{F})₂ (e.g., wherein R^{F} is hydrogen or optionally substituted alkyl, or optionally substituted phenyl). In some embodiments, R^{55a} is —OH. In some embodiments, R^{55a} is —SH. In some embodiments, R^{55a} is —NH₂. In some embodiments, R^{55a} is —CN, —SCN, —SSR^{F}, —N₃, —NO, or —NO₂. In some embodiments, R^{55a} is —C(═O)R^{F}, —C(═O)OR^{F}, —C(═O)SR^{F}, or —C(═O)N(R^{E})₂ (e.g., wherein R^{F} is hydrogen or optionally substituted alkyl, or optionally substituted phenyl). In some embodiments, R^{55a} is —C(═NR^{F})R^{F}, —C(═NR^{F})OR^{F}, —C(═NR^{F})SR^{F}, or —C(═NR^{F})N(R^{F})₂ (e.g., wherein R^{F} is hydrogen or optionally substituted alkyl, or optionally substituted phenyl). In some embodiments, R^{55a} is —S(═O)R^{F}, —S(═O)OR^{F}, —S(═O)SR^{F}, —S(═O)N(R^{F})₂, —S(═O)₂R^{F}, —S(═O)₂OR^{F}, —S(═O)₂SR^{F}, or —S(═O)₂N(R^{F})₂ (e.g., wherein R^{F} is hydrogen or optionally substituted alkyl, or optionally substituted phenyl). In some embodiments, R^{55a} is —OC(═O)R^{F}, —OC(═O)OR^{F}, —OC(═O)SR^{F}, —OC(═O)N(R^{F})₂, —OC(═NR^{F})R^{F}, —OC(═NR^{F})OR^{F}, —OC(═NR^{F})SR^{F}, —OC(═NR^{F})N(R^{F})₂, —OS(═O)R^{F}, —OS(═O)OR^{F}, —OS(═O)SR^{F}, —OS(═O)N(R^{F})₂, —OS(═O)₂R^{F}, —OS(═O)₂OR^{F}, —OS(═O)₂SR^{F}, —OS(═O)₂N(R^{F})₂, or —ON(R^{F})₂ (e.g., wherein R^{F} is hydrogen or optionally substituted alkyl, or optionally substituted phenyl). In some embodiments, R^{55a} is —SC(═O)R^{F}, —SC(═O)OR^{F}, —SC(═O)SR^{F}, —SC(═O)N(R^{F})₂, —SC(═NR^{F})R^{F}, —SC(═NR^{F})OR^{F}, —SC(═NR^{F})SR^{F}, or —SC(═NR^{F})N(R^{E})₂ (e.g., wherein R^{F} is hydrogen or optionally substituted alkyl, or optionally substituted phenyl). In some embodiments, R^{55a} is —NR^{F}C(═O)R^{F}, —NR^{F}C(═O)OR^{F}, —NR^{F}C(═O)SR^{F}, —NR^{F}C(═O)N(R^{F})₂, —NR^{F}C(═NR^{F})R^{F}, —NR^{F}C(═NR^{F})OR^{F}, —NR^{F}C(═NR^{F})SR^{F}, —NR^{F}C(═NR^{F})N(R^{F})₂, —NR^{F}S(═O)R^{F}, —NR^{F}S(═O)OR^{F}, —NR^{F}S(═O)SR^{F}, —NR^{F}S(═O)N(R^{F})₂, —NR^{F}S(═O)₂R^{F}, —NR^{F}S(═O)₂OR^{F}, —NR^{F}S(═O)₂SR^{F}, or —NR^{F}S(═O)₂N(R^{F})₂ (e.g., wherein R^{F} is hydrogen or optionally substituted alkyl, or optionally substituted phenyl). In some embodiments, R^{35a} is —Si(R^{F})₃, —Si(R^{F})₂OR^{F}, —Si(R^{F})(OR^{F})₂, —Si(OR^{F})₃, —OSi(R^{F})₃, —OSi(R^{F})₂OR^{F}, —OSi(R^{F})(OR^{F})₂, or —OSi(OR^{F})₃ (e.g., wherein R^{F} is hydrogen or optionally substituted alkyl, or optionally substituted phenyl).

In some embodiments, R^{55a} is optionally substituted carbocyclyl, optionally substituted heterocyclyl, optionally substituted aryl, or optionally substituted heteroaryl. In some embodiments, R^{55a} is optionally substituted C₃₋₁₄ carbocyclyl. In some embodiments, R^{55a} is optionally substituted monocyclic C₃₋₇ carbocyclyl. In some embodiments, R^{55a} is optionally substituted monocyclic C₃₋₄ carbocyclyl. In some embodiments, R^{55a} is optionally substituted monocyclic C₅₋₇ carbocyclyl. In some embodiments, R^{55a} is saturated carbocyclyl. In some embodiments, R^{55a} is carbocyclyl comprising only one unsaturated bond (e.g., C═C bond) in the carbocyclic ring system.

In some embodiments, R^{55a} is optionally substituted 3- to 14-membered heterocyclyl. In some embodiments, R^{55a} is optionally substituted monocyclic 3- to 7-membered heterocyclyl. In some embodiments, R^{55a} is optionally substituted 3- to 14-membered heterocyclyl comprising one or more O and/or S atoms but no N atoms. In some embodiments, R^{55a} is optionally substituted 3- to 14-membered heterocyclyl comprising one or more N atoms and optionally one or more O and/S atoms. In some embodiments, R^{55a} is optionally substituted monocyclic 3- to 7-membered heterocyclyl comprising one or more O and/or S atoms but no N atoms. In some embodiments, R^{55a} is optionally substituted monocyclic 3- to 7-membered heterocyclyl comprising one or more N atoms and optionally one or more O and/or S atoms. In some embodiments, R^{55a} is saturated heterocyclyl. In some embodiments, R^{55a} is heterocyclyl comprising only one unsaturated bond (e.g., C═C bond) in the heterocyclic ring system.

In some embodiments, R^{55a} is optionally substituted monocyclic aryl. In some embodiments, R^{55a} is optionally substituted bicyclic aryl. In some embodiments, R^{55a} is optionally substituted C₆₋₁₄ aryl. In some embodiments, R^{55a} is optionally substituted C₆₋₁₀ aryl. In some embodiments, R^{55a} is optionally substituted phenyl. In some embodiments, R^{55a} is optionally substituted naphthyl.

In some embodiments, R^{55a} is optionally substituted monocyclic heteroaryl. In some embodiments, R^{55a} is optionally substituted bicyclic heteroaryl. In some embodiments, R^{55a} is optionally substituted 5- to 14-membered heteroaryl. In some embodiments, R^{55a} is optionally substituted 5- to 10-membered heteroaryl. In some embodiments, R^{55a} is optionally substituted 5- to 6-membered monocyclic heteroaryl.

In some embodiments, R^{55b} is halogen, optionally substituted alkyl, optionally substituted alkenyl, optionally substituted alkynyl, optionally substituted heteroalkyl, optionally substituted heteroalkenyl, optionally substituted heteroalkynyl, —OR^{F}, —SR^{F}, or —N(R^{F})₂. In some embodiments, R^{55b} is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R^{55b} is C₁₋₁₀ haloalkyl. In some embodiments, R^{55b} is C₁₋₄ haloalkyl. In some embodiments, R^{55b} is C₁₋₄ fluoroalkyl (e.g., C₁₋₄ perfluoroalkyl). In some embodiments, R^{55b} is —CF₃. In some embodiments, R^{55b} is hydrogen, optionally substituted C₁-C₆ alkyl, or halogen. In some embodiments, R^{55b} is hydrogen, fluorine, —CH₃, —CH₂F, —CHF₂, or —CF₃. In some embodiments, R^{55b} is hydrogen, fluorine, —CH₃, or —CF₃. In some embodiments, R^{55b} is hydrogen or halogen. In some embodiments, R^{55b} is hydrogen or fluorine. In some embodiments, R^{55b} is hydrogen or optionally substituted C₁-C₆ alkyl. In some embodiments, R^{55b} is hydrogen, unsubstituted C₁-C₆ alkyl, or C₁₋₆ haloalkyl. In some embodiments, R^{55b} is hydrogen.

In some embodiments, R^{55b} is halogen. In some embodiments, R^{55b} is bromine, chlorine, or fluorine. In some embodiments, R^{55b} is bromine or chlorine. In some embodiments, R^{55b} is chlorine or fluorine. In some embodiments, R^{55b} is bromine. In some embodiments, R^{55b} is chlorine. In some embodiments, R^{55b} is fluorine.

In some embodiments, R^{55b} is —OR^{F}, —SR^{F}, or —N(R^{F})₂ (e.g., wherein R^{F} is hydrogen or optionally substituted alkyl, or optionally substituted phenyl). In some embodiments, R^{55b} is —OH. In some embodiments, R^{55b} is —SH. In some embodiments, R^{55b} is —NH₂. In some embodiments, R^{55b} is —CN, —SCN, —SSR^{F}, —N₃, —NO, or —NO₂. In some embodiments, R^{55b} is —C(═O)R^{F}, —C(═O)OR^{F}, —C(═O)SR^{F}, or —C(═O)N(R^{F})₃ (e.g., wherein R^{F} is hydrogen or optionally substituted alkyl, or optionally substituted phenyl). In some embodiments, R^{55b} is —C(═NR^{F})R^{F}, —C(═NR^{F})OR^{F}, —C(═NR^{F})SR^{F}, or —C(═NR^{F})N(R^{F})₂ (e.g., wherein R^{F} is hydrogen or optionally substituted alkyl, or optionally substituted phenyl). In some embodiments, R^{55b} is —S(═O)R^{F}, —S(═O)OR^{F}, —S(═O)SR^{F}, —S(═O)N(R^{F})₂, —S(═O)₂R^{F}, —S(═O)₂OR^{F}, —S(═O)₂SR^{F}, or —S(═O)₂N(R^{F})₂ (e.g., wherein R^{F} is hydrogen or optionally substituted alkyl, or optionally substituted phenyl). In some embodiments, R^{55b} is —OC(═O)R^{F}, —OC(═O)OR^{F}, —OC(═O)SR^{F}, —OC(═O)N(R^{F})₂, —OC(═NR^{F})R^{F}, —OC(═NR^{F})OR^{F}, —OC(═NR^{F})SR^{F}, —OC(═NR^{F})N(R^{F})₂, —OS(═O)R^{F}, —OS(═O)OR^{F}, —OS(═O)SR^{F}, —OS(═O)N(R^{E})₂, —OS(═O)₂R^{F}, —OS(═O)₂OR^{F}, —OS(═O)₂SR^{F}, —OS(═O)₂N(R^{F})₂, or —ON(R^{F})₂ (e.g., wherein R^{F} is hydrogen or optionally substituted alkyl, or optionally substituted phenyl). In some embodiments, R^{55b} is —SC(═O)R^{F}, —SC(═O)OR^{F}, —SC(═O)SR^{F}, —SC(═O)N(R^{F})₂, —SC(═NR^{F})R^{F}, —SC(═NR^{F})OR^{F}, —SC(═NR^{F})SR^{F}, or —SC(═NR^{F})N(R^{F})₂ (e.g., wherein R^{F} is hydrogen or optionally substituted alkyl, or optionally substituted phenyl). In some embodiments, R^{55b} is —NR^{F}C(═O)R^{F}, —NR^{F}C(═O)OR^{F}, —NR^{F}C(═O)SR^{F}, —NR^{F}C(═O)N(R^{F})₂, —NR^{F}C(═NR^{F})R^{F}, —NR^{F}C(═NR^{F})OR^{F}, —NR^{F}C(═NR^{F})SR^{F}, —NR^{F}C(═NR^{F})N(R^{F})₂, —NR^{F}S(═O)R^{F}, —NR^{F}S(═O)OR^{F}, —NR^{F}S(═O)SR^{F}, —NR^{F}S(═O)N(R^{F})₂, —NR^{F}S(═O)₂R^{F}, —NR^{F}S(═O)₂OR^{F}, —NR^{F}S(═O)₂SR^{F}, or —NR^{F}S(═O)₂N(R^{F})₂ (e.g., wherein R^{F} is hydrogen or optionally substituted alkyl, or optionally substituted phenyl). In some embodiments, R^{55b} is —Si(R^{F})₂, —Si(R^{F})₂OR^{F}, —Si(R^{F})(OR^{F})₂, —Si(OR^{F})₂, —OSi(R^{F})₃, —OSi(R^{F})₂OR^{F}, —OSi(R^{F})(OR^{F})₂, or —OSi(OR^{F})₃ (e.g., wherein R^{F} is hydrogen or optionally substituted alkyl, or optionally substituted phenyl).

In some embodiments, R^{55b} is optionally substituted carbocyclyl, optionally substituted heterocyclyl, optionally substituted aryl, or optionally substituted heteroaryl. In some embodiments, R^{55b} is optionally substituted C₃₋₁₄ carbocyclyl. In some embodiments, R^{55b} is optionally substituted monocyclic C₃₋₇ carbocyclyl. In some embodiments, R^{55b} is optionally substituted monocyclic C₃₋₄ carbocyclyl. In some embodiments, R^{55b} is optionally substituted monocyclic C₅₋₇ carbocyclyl. In some embodiments, R^{55b} is saturated carbocyclyl. In some embodiments, R^{35b} is carbocyclyl comprising only one unsaturated bond (e.g., C═C bond) in the carbocyclic ring system.

In some embodiments, R^{55b} is optionally substituted 3- to 14-membered heterocyclyl. In some embodiments, R^{55b} is optionally substituted monocyclic 3- to 7-membered heterocyclyl. In some embodiments, R^{55b} is optionally substituted 3- to 14-membered heterocyclyl comprising one or more O and/or S atoms but no N atoms. In some embodiments, R^{55b} is optionally substituted 3- to 14-membered heterocyclyl comprising one or more N atoms and optionally one or more O and/S atoms. In some embodiments, R^{55b} is optionally substituted monocyclic 3- to 7-membered heterocyclyl comprising one or more O and/or S atoms but no N atoms. In some embodiments, R^{55b} is optionally substituted monocyclic 3- to 7-membered heterocyclyl comprising one or more N atoms and optionally one or more O and/or S atoms. In some embodiments, R^{55b} is saturated heterocyclyl. In some embodiments, R^{55b} is heterocyclyl comprising only one unsaturated bond (e.g., C═C bond) in the heterocyclic ring system.

In some embodiments, R^{55b} is optionally substituted monocyclic aryl. In some embodiments, R^{55b} is optionally substituted bicyclic aryl. In some embodiments, R^{35b} is optionally substituted C₆₋₁₄ aryl. In some embodiments, R^{55b} is optionally substituted C₆₋₁₀ aryl. In some embodiments, R^{55b} is optionally substituted phenyl. In some embodiments, R^{55b} is optionally substituted naphthyl.

In some embodiments, R^{55b} is optionally substituted monocyclic heteroaryl. In some embodiments, R^{55b} is optionally substituted bicyclic heteroaryl. In some embodiments, R^{55b} is optionally substituted 5- to 14-membered heteroaryl. In some embodiments, R^{55b} is optionally substituted 5- to 10-membered heteroaryl. In some embodiments, R^{55b} is optionally substituted 5- to 6-membered monocyclic heteroaryl.

In some embodiments, R^{54a} and R^{54b} are taken together with their intervening atom to form an optionally substituted, monocyclic, carbocyclic or heterocyclic ring. In some embodiments, R^{54a} and R^{54b} are taken together with their intervening atom to form an optionally substituted, monocyclic, C₃₋₁₄ carbocyclic ring. In some embodiments, R^{54a} and R^{54b} are taken together with their intervening atom to form an optionally substituted, monocyclic, C₃₋₇ carbocyclic ring. In some embodiments, R^{54a} and R^{54b} are taken together with their intervening atom to form an optionally substituted, monocyclic, 3- to 14-membered heterocyclic ring. In some embodiments, R^{54a} and R^{54b} are taken together with their intervening atom to form an optionally substituted, monocyclic, 3- to 7-membered heterocyclic ring.

In some embodiments, R^{54b} and R^{55a} are taken together with their intervening atom to form an optionally substituted, monocyclic, carbocyclic or heterocyclic ring. In some embodiments, R^{54b} and R^{55a} are taken together with their intervening atom to form an optionally substituted, monocyclic, C₃₋₁₄ carbocyclic ring. In some embodiments, R^{54b} and R^{55a} are taken together with their intervening atom to form an optionally substituted, monocyclic, C₃₋₇ carbocyclic ring. In some embodiments, R^{54b} and R^{55a} are taken together with their intervening atom to form an optionally substituted, monocyclic, 3- to 14-membered heterocyclic ring. In some embodiments, R^{54b} and R^{55a} are taken together with their intervening atom to form an optionally substituted, monocyclic, 3- to 7-membered heterocyclic ring.

In some embodiments, R^{55a} and R^{55b} are taken together with their intervening atom to form an optionally substituted, monocyclic, 5- to 7-membered heterocyclic ring. In some embodiments, R^{55a} and R^{55b} are taken together with their intervening atom to form an optionally substituted, monocyclic, 5- to 6-membered heterocyclic ring. In some embodiments, R^{55a} and R^{55b} are taken together with their intervening atom to form an optionally substituted, monocyclic, 6- to 7-membered heterocyclic ring. In some embodiments, R^{55a} and R^{55b} are taken together with their intervening atom to form an optionally substituted, monocyclic, 5-membered heterocyclic ring. In some embodiments, R^{55a} and R^{55b} are taken together with their intervening atom to form an optionally substituted, monocyclic, 6-membered heterocyclic ring. In some embodiments, R^{55a} and R^{55b} are taken together with their intervening atom to form an optionally substituted, monocyclic, 7-membered heterocyclic ring. In some embodiments, R^{55a} and R^{55b} are taken together with their intervening atom to form optionally substituted tetrahydrofuranyl, optionally substituted tetrahydropyranyl ring, optionally substituted pyrrolidinyl, or optionally substituted piperidinyl.

In some embodiments, R^{55a} and R^{55b} are taken together with their intervening atom to form an optionally substituted, monocyclic, 5- to 7-membered heterocyclic ring comprising one or more O and/or S atoms but no N atoms. In some embodiments, R^{55a} and R^{55b} are taken together with their intervening atom to form optionally substituted tetrahydrofuranyl or optionally substituted tetrahydropyranyl. In some embodiments, R^{55a} and R^{55b} are taken together with their intervening atom to form optionally substituted tetrahydrofuranyl. In some embodiments, R^{55a} and R^{55b} are taken together with their intervening atom to form optionally substituted tetrahydropyranyl.

In some embodiments, R^{55a} and R^{55b} are taken together with their intervening atom to form an optionally substituted, monocyclic, 5- to 7-membered heterocyclic ring comprising one or more N atoms. In some embodiments, R^{55a} and R^{55b} are taken together with their intervening atom to form an optionally substituted, monocyclic, 5- to 7-membered heterocyclic ring comprising one or more N atoms. In some embodiments, R^{55a} and R^{35b} are taken together with their intervening atom to form optionally substituted pyrrolidinyl or optionally substituted piperidinyl. In some embodiments, R^{55a} and R^{55b} are taken together with their intervening atom to form optionally substituted pyrrolidinyl. In some embodiments, R^{55a} and R^{55b} are taken together with their intervening atom to form optionally substituted piperidinyl.

In some embodiments, R^{55b} and R^{56a} are taken together with their intervening atom to form an optionally substituted, monocyclic, carbocyclic or heterocyclic ring. In some embodiments, R^{56b} and R^{56a} are taken together with their intervening atom to form an optionally substituted, monocyclic, C₃₋₁₄ carbocyclic ring. In some embodiments, R^{55b} and R^{56a} are taken together with their intervening atom to form an optionally substituted, monocyclic, C₃₋₇ carbocyclic ring. In some embodiments, R^{55b} and R^{56a} are taken together with their intervening atom to form an optionally substituted, monocyclic, 3- to 14-membered heterocyclic ring. In some embodiments, R^{55b} and R^{56a} are taken together with their intervening atom to form an optionally subsituted, monocyclic, 3- to 7-membered heterocyclic ring.

In some embodiments, R^{56a} and R^{56b} are taken together with their intervening atom to form an optionally substituted, monocyclic, carbocyclic or heterocyclic ring. In some embodiments, R^{56a} and R^{56b} are taken together with their intervening atom to form an optionally substituted, monocyclic, C₃₋₁₄ carbocyclic ring. In some embodiments, R^{56a} and R^{56b} are taken together with their intervening atom to form an optionally substituted, monocyclic, C₃₋₇ carbocyclic ring. In some embodiments, R^{56a} and R^{56b} are taken together with their intervening atom to form an optionally substituted, monocyclic, 3- to 14-membered heterocyclic ring. In some embodiments, R^{56a} and R^{56b} are taken together with their intervening atom to form an optionally substituted, monocyclic, 3- to 7-membered heterocyclic ring.

In some embodiments, Formula VI-A or VI-B is any one of the formulae shown in Table 11:

TABLE 11

| | |
|---|---|
| 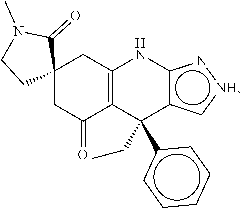 | 949753 |
| 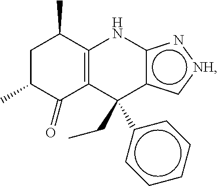 | 865199 |
| 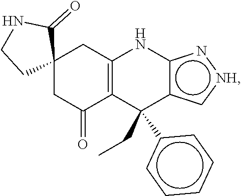 | 950161 |
| 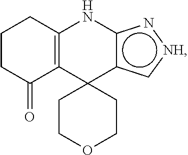 | 884111 |
| 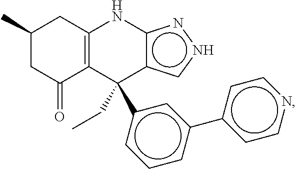 | 874315 |
| 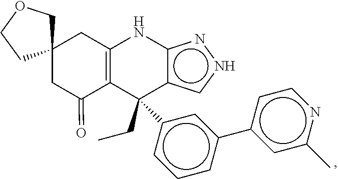 | 947412 |
| 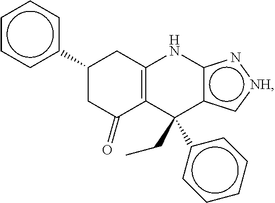 | 791265 |
| 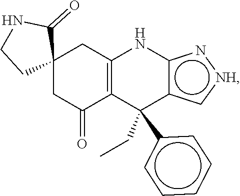 | 950160 |
| 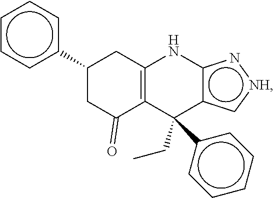 | 791264 |
| 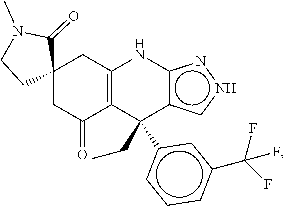 | 950330 |
| 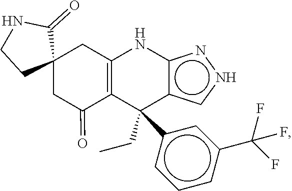 | 950331 |
| 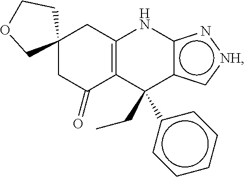 | 816884 |
| 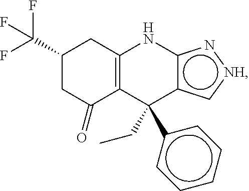 | 806608 |
| 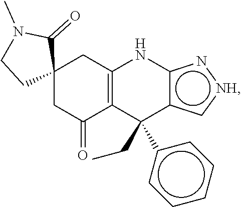 | 949760 |
| 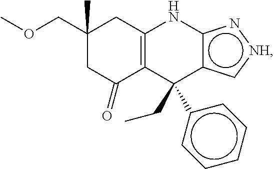 | 816875 |
| 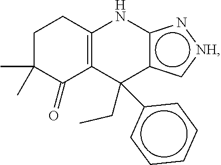 | 835310 |
| 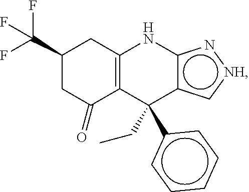 | 806609 |
| 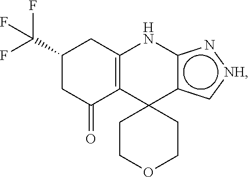 | 901477 |
| 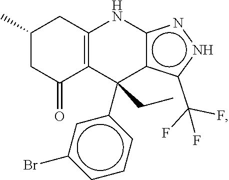 | 828849 |
| 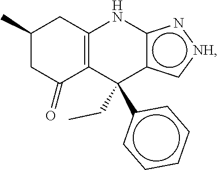 | 789013 |
| 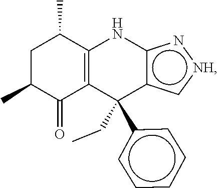 | 865198 |
| 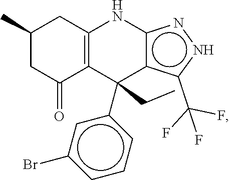 | 828850 |
| 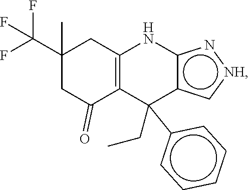 | 816796 |
| 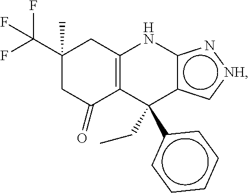 | 819569 |
| 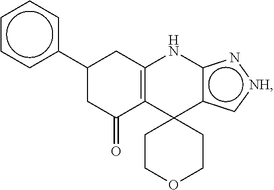 | 893309 |
| 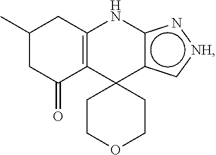 | 884112 |
| 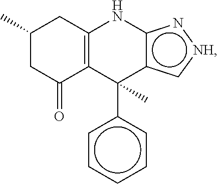 | 789334 |
| 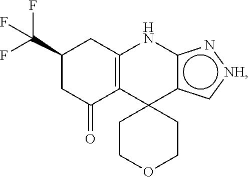 | 901478 |
| 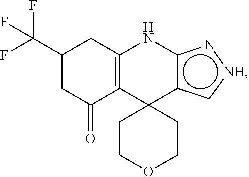 | 889572 |
| 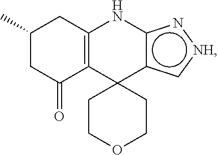 | 912078 |
| 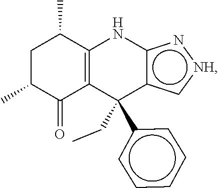 | 909217 |
| 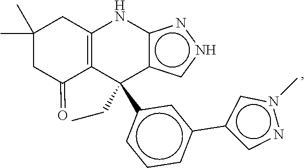 | 823724 |
| 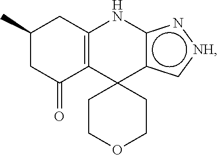 | 912079 |
| 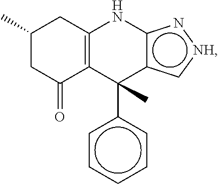 | 789332 |
| 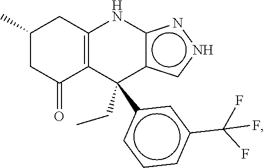 | 810452 |
| 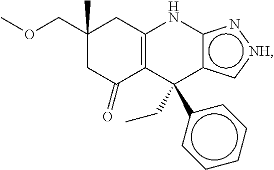 | 816873 |
| 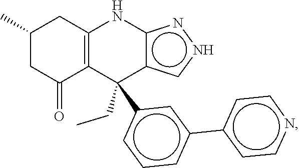 | 874316 |
| 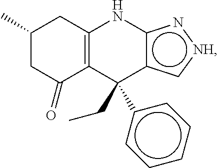 | 789010 |
| 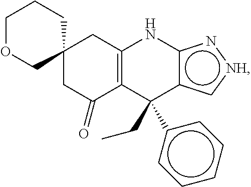 | 816878 |
| 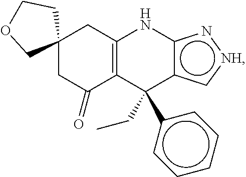 | 816881 |
| 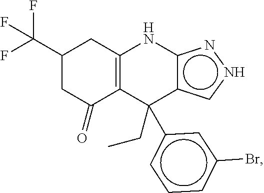 | 819565 |
| 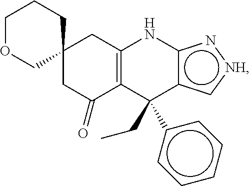 | 816879 |
| 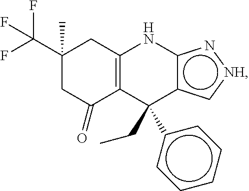 | 819568 |
| 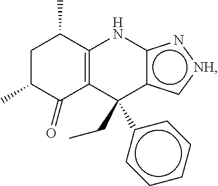 | 909216 |
| 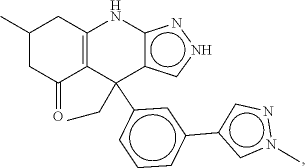 | 816752 |
| 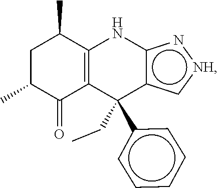 | 865201 |
| 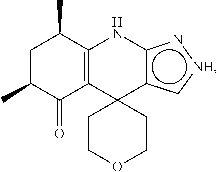 | 893316 |
| 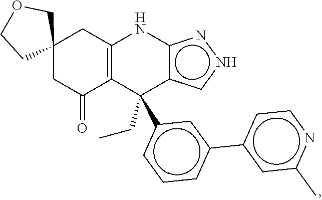 | 947407 |
| 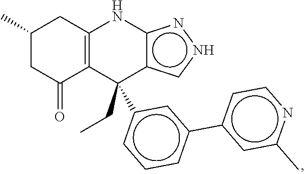 | 932657 |
| 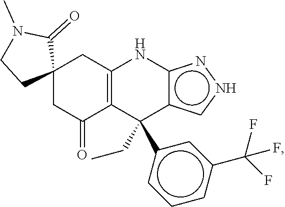 | 950326 |
| 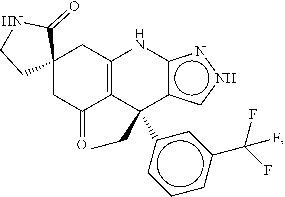 | 950333 |
| 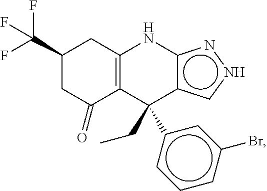 | 826653 |
| 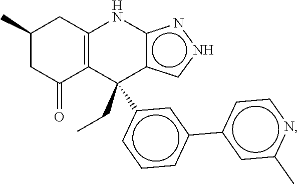 | 932658 |
| 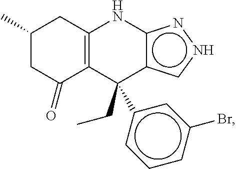 | 810486 |
| 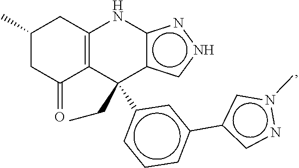 | 823720 |
| 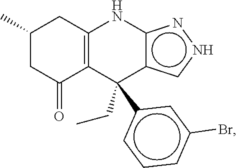 | 810487 |
| 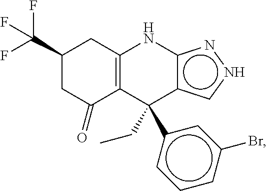 | 826652 |
| 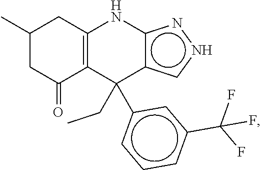 | 801397 |
| 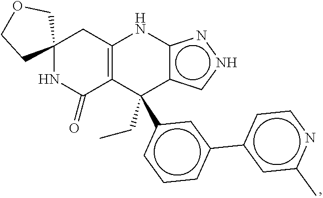 | 947406 |
| 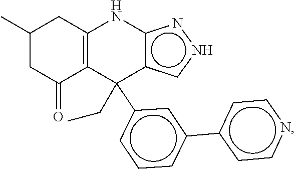 | 856947 |
| 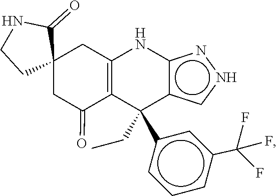 | 950324 |
| 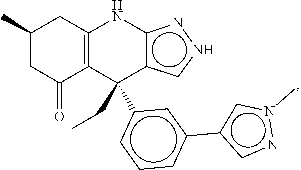 | 823721 |
| 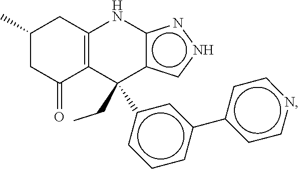 | 874318 |
| 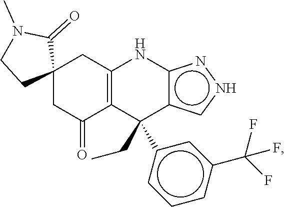 | 950329 |
| 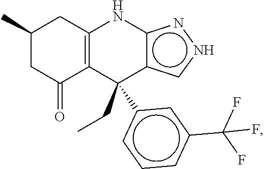 | 810451 |
| 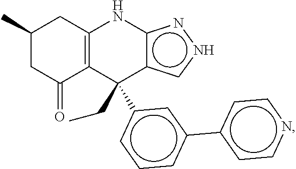 | 874317 |
| 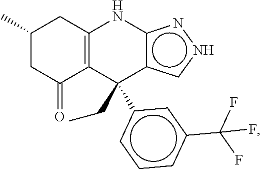 | 810450 |
| 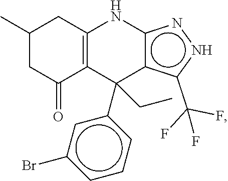 | 826715 |
| 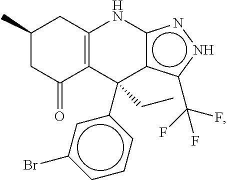 | 828848 |
| 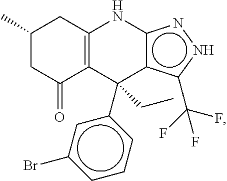 | 828847 |
| 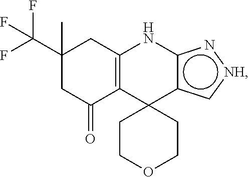 | 901458 |
| 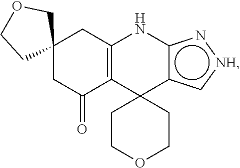 | 901476 |
| 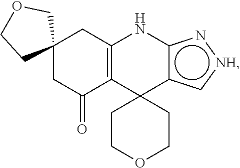 | 901475 |
| 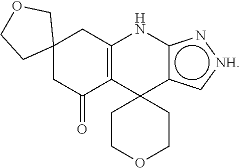 | 889573 |

In some embodiments, Formula VI-A or VI-B is any one of the formulae shown in Table 12:

TABLE 12

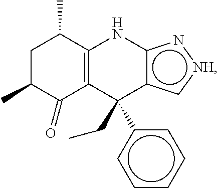

865202

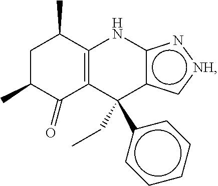

909218

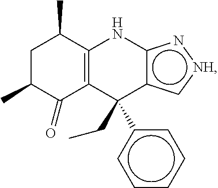

909219

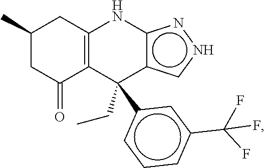

810453

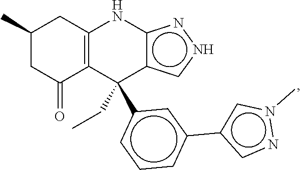

823726

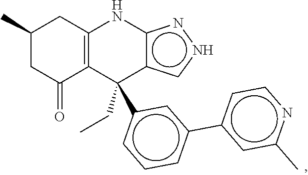

932637

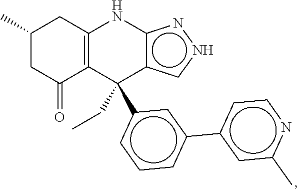

932638

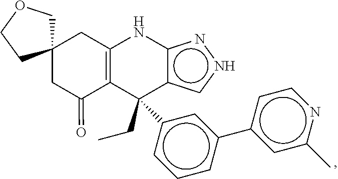

947411

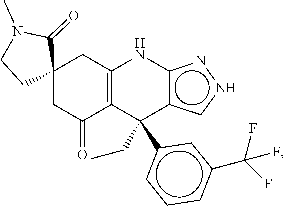

950325

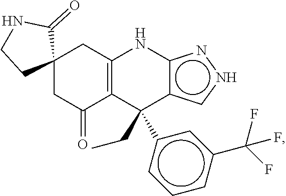

950332

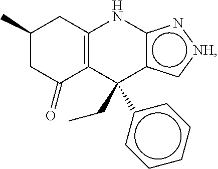

789009

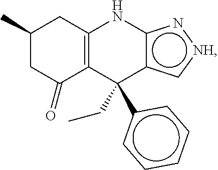

789011

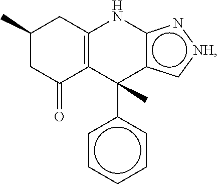

789331

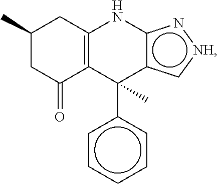

789333

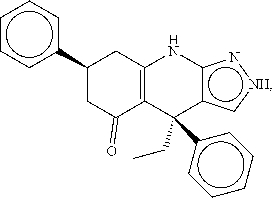

791262

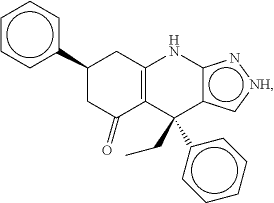

791263

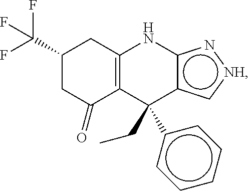

806607

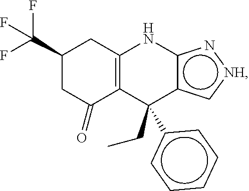

806611

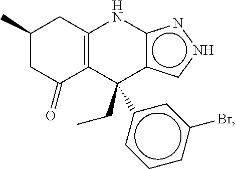

810484

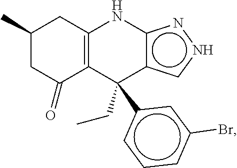

810485

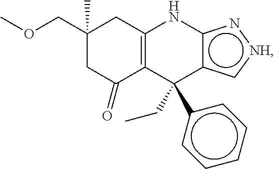

816872

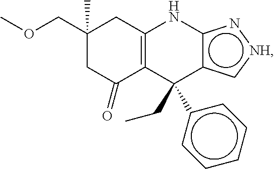

816874

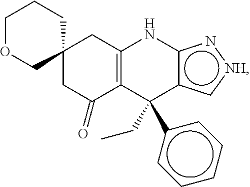

816876

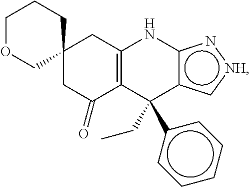

816877

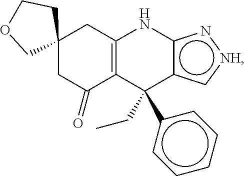

816880

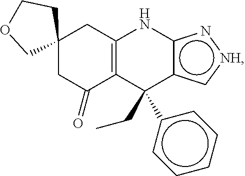

816882

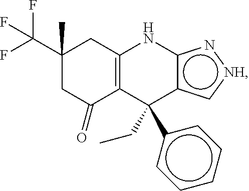

819570

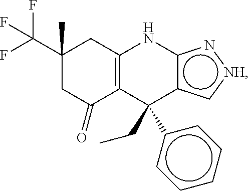

819571

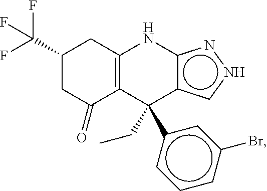

826654

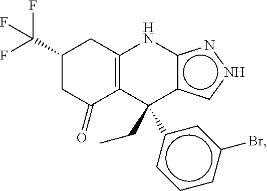

826655

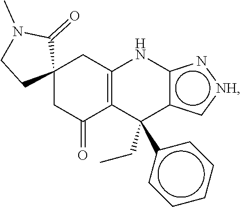

949754

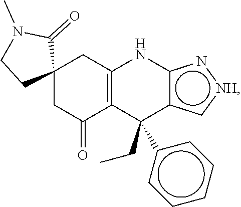

949759

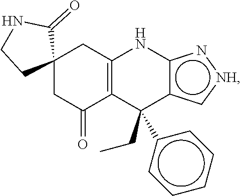

950164

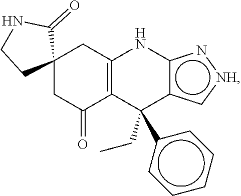

950165.

In some embodiments, Formula VI-A or VI-B is not any one of the formulae shown in Table 12.

In another aspect, provided herein is a compound of any one of the formulae shown in Table 13, or a pharmaceutically acceptable salt, solvate, hydrate, polymorph, co-crystal, tautomer, stereoisomer, isotopically labeled compound, or prodrug thereof;

TABLE 13

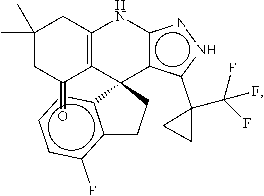

816839

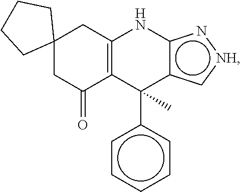

792339

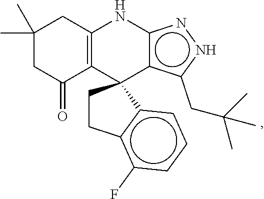

791256

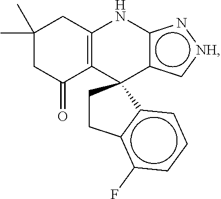

806632

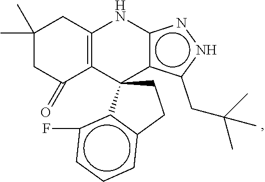

801351

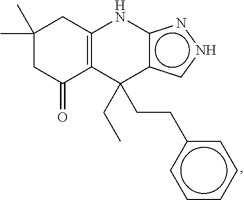

819563

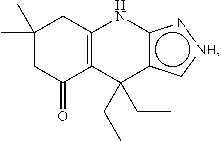

810505

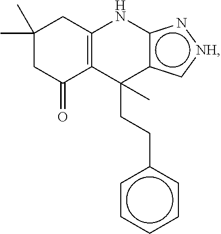

819562

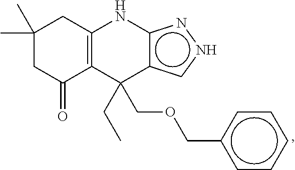

874313

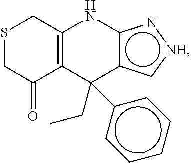

801406

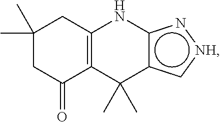

786065

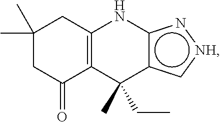

884019

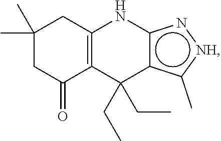

786061

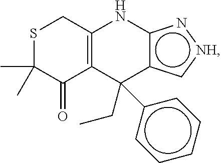

862028

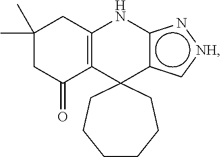

883978

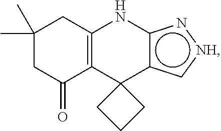

874509

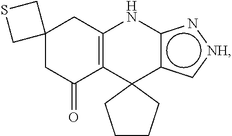

913441

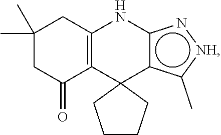

786072

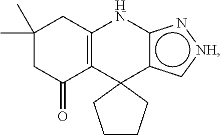

790666

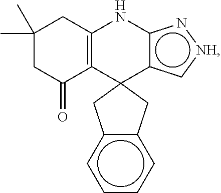

897752

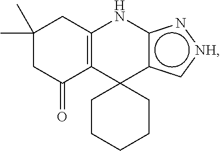

826650

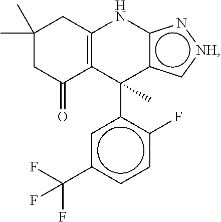

816797

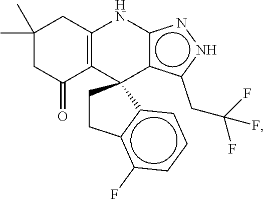

816837

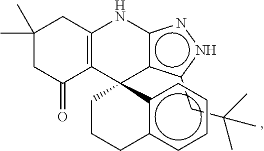

811596

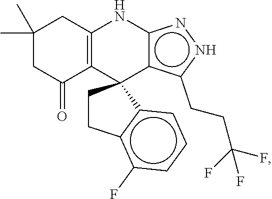

874323

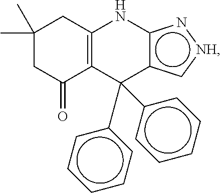

789461

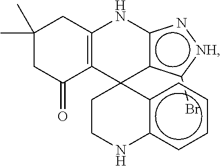

810460

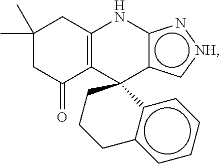

930565

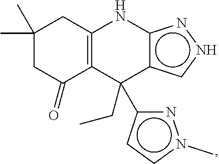

790619

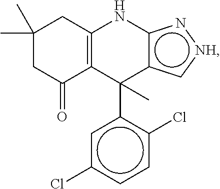

806605

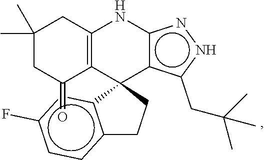

789504

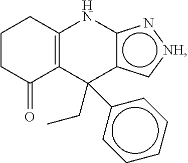

789497

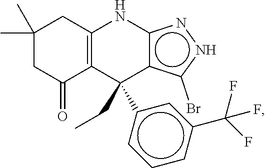

810464

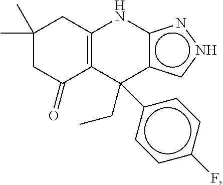

772965

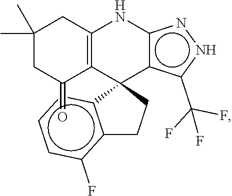

806568

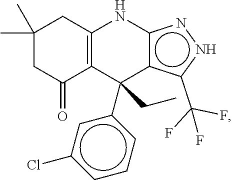

801309

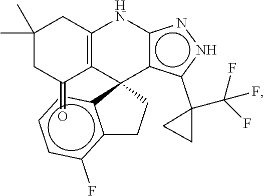

816840

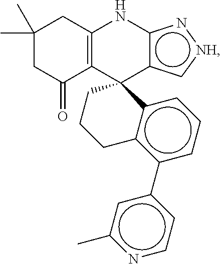

945550

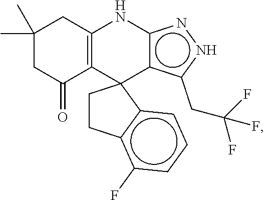

810590

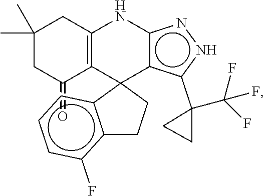

810591

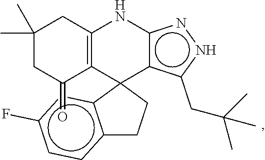

784440

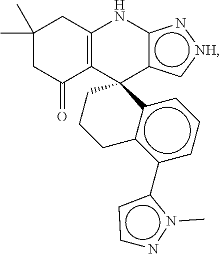

945552

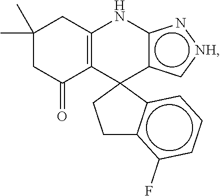

801357

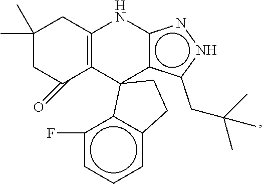

789583

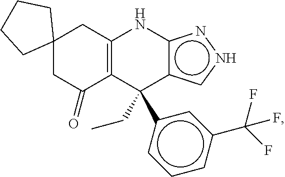

801310

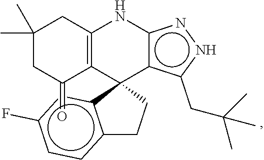

789505

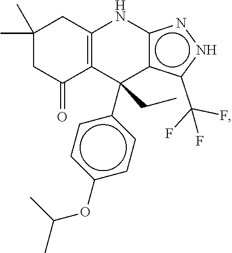

947873

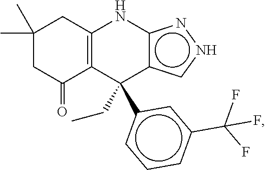

790755

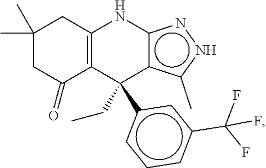

810368

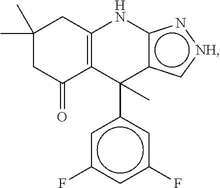

785735

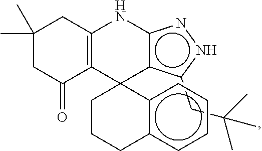

810495

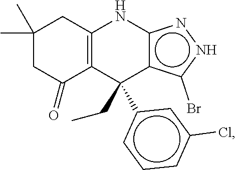

801315

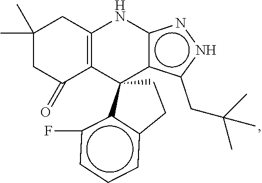

801352

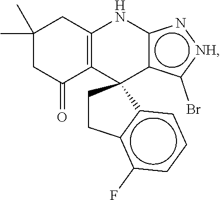

806938

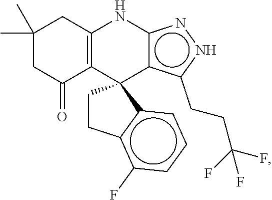

874324

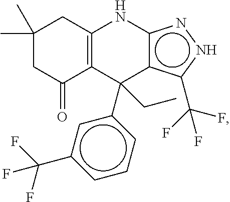

789584

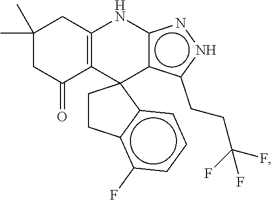

841868

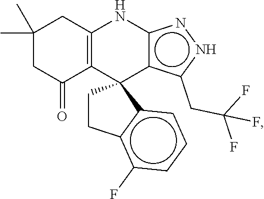

816838

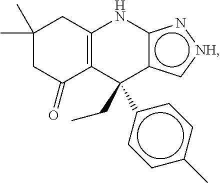

789457

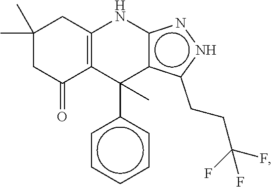

828845

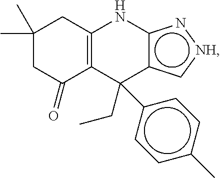

785739

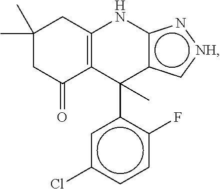

810377

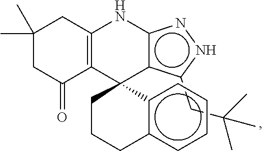

811595

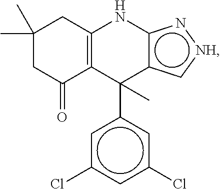

789337

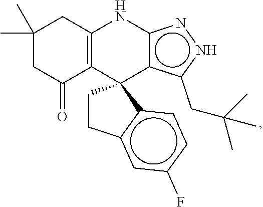

782425

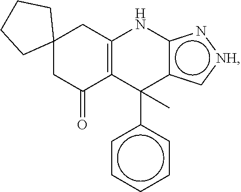

789460

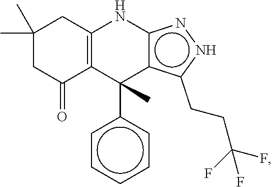

856949

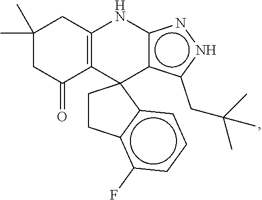

789524

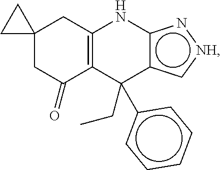

789330

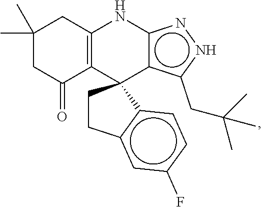

782424

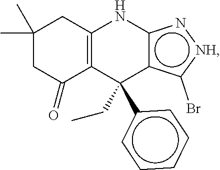

792341

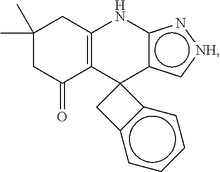

917226

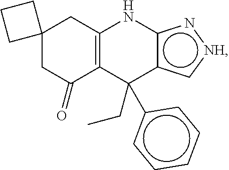

811555

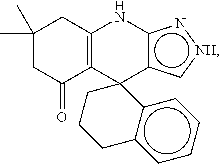

913219

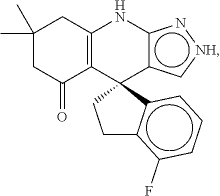

806633

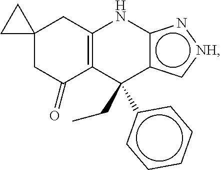

790615

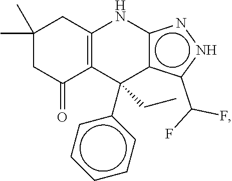

874511

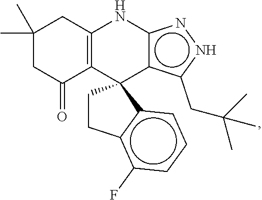

791257

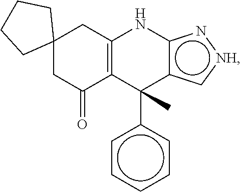

792338

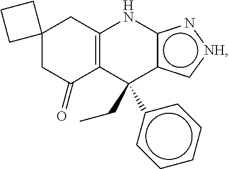

819573

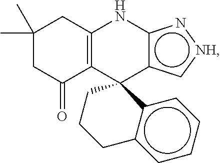

930566

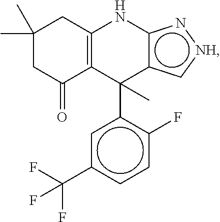

810376

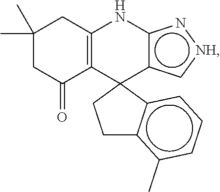

806603

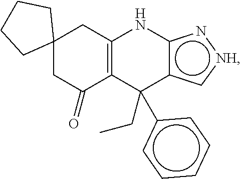

785737

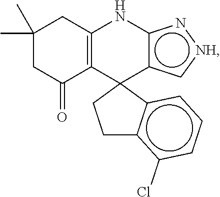

816795

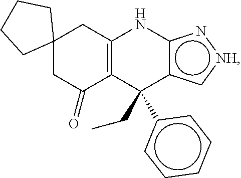

789562

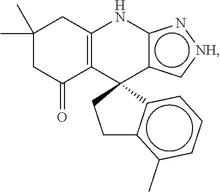

913136

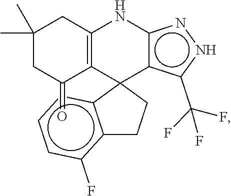

801356

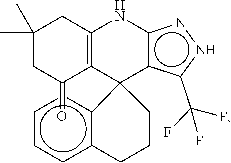

790745

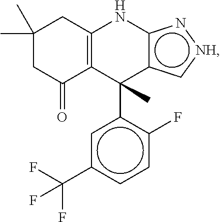

816798

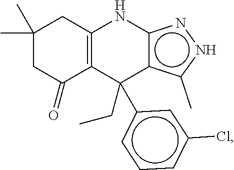

789336

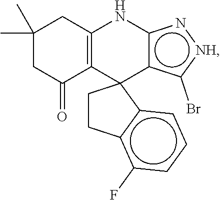

801358

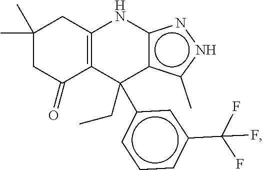

801321

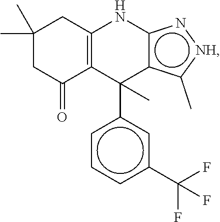

811556

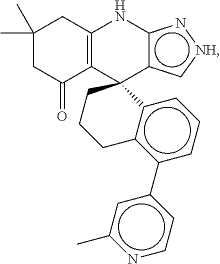

945549

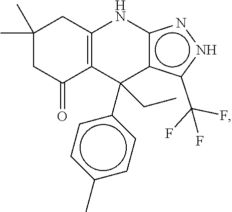

883866

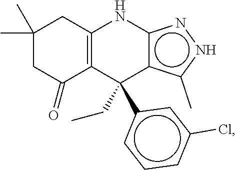

790612

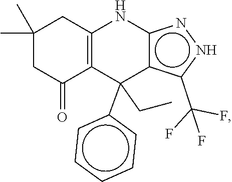

792343

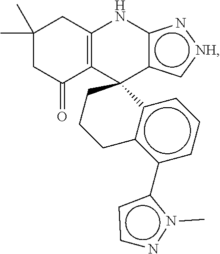

945551

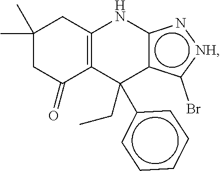

789459

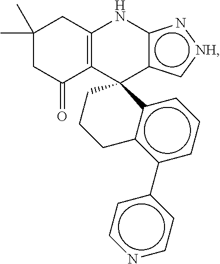

945548

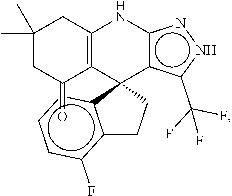

806569

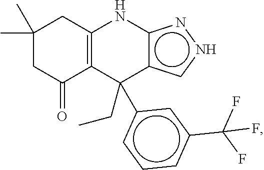

785762

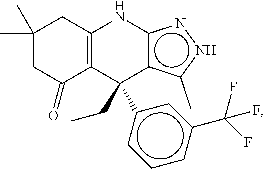

810367

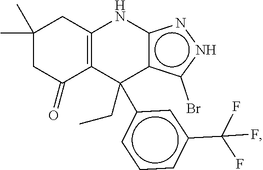

802358

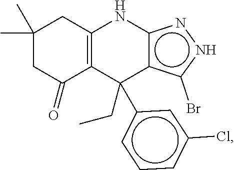

789564

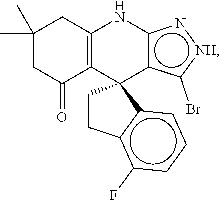

806939

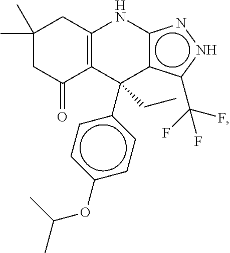

947868

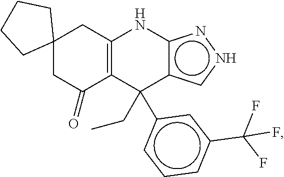

790617

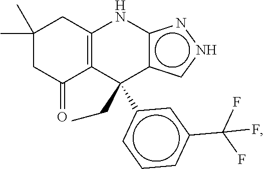

790754

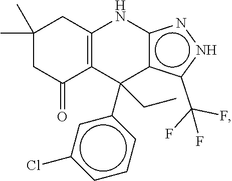

789566

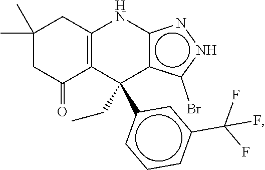

810466

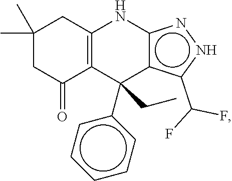

874510

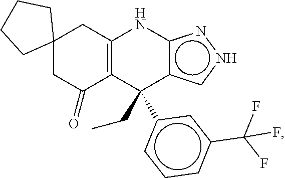

801311

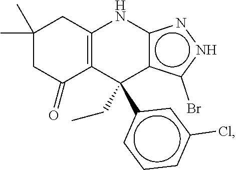

801314

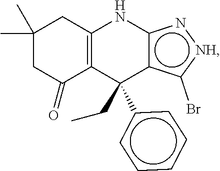

792340

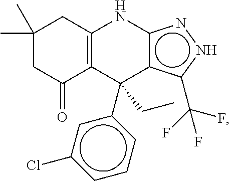

801308

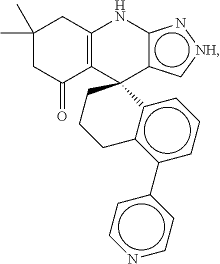

945547

In another aspect, provided herein is a compound of any one of the formulae shown in Table 13A, or a pharmaceutically acceptable salt, solvate, hydrate, polymorph, co-crystal, tautomer, stereoisomer, isotopically labeled compound, or prodrug thereof:

TABLE 13A

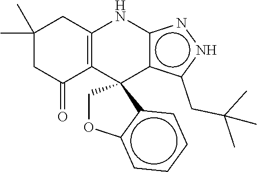

117093,

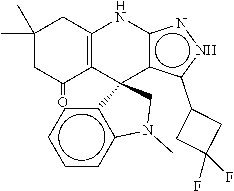

119047,

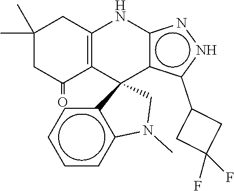

119048,

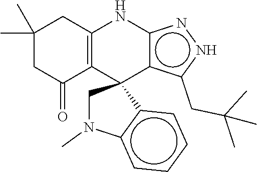

125978,

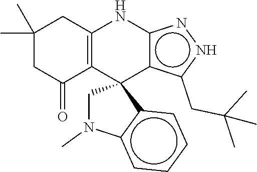

125979,

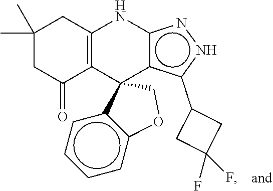

127429

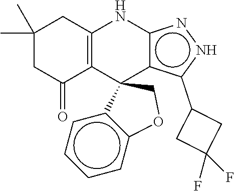

127430.

In another aspect, provided herein is a compound of any one of the formulae shown in Table 14, or a pharmaceutically acceptable salt, solvate, hydrate, polymorph, co-crystal, tautomer, stereoisomer, isotopically labeled compound, or prodrug thereof:

TABLE 14

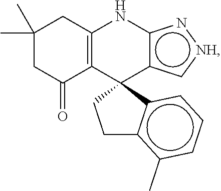

913137

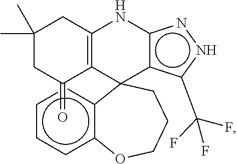

790632

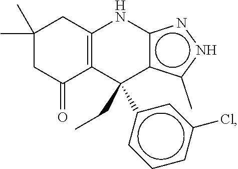

790611

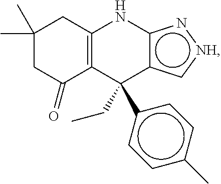

789458

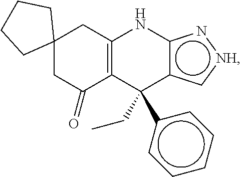

789563

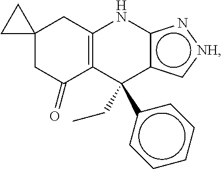

790616

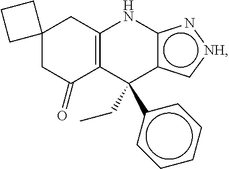

819572

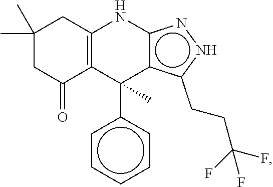

856948

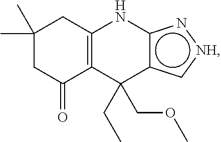

841866

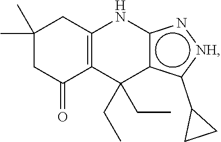

877294

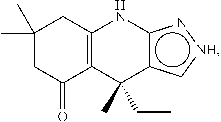

884020

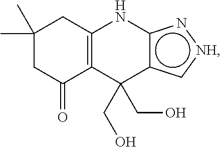

889570

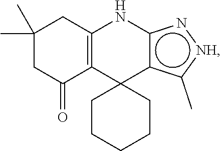

877288

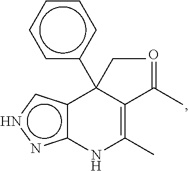

806940

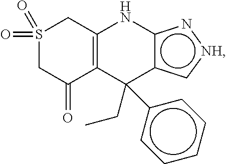

811512

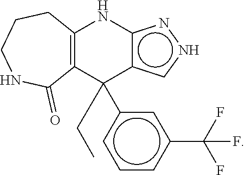

913355

In another aspect, provided herein is a compound of the formula:

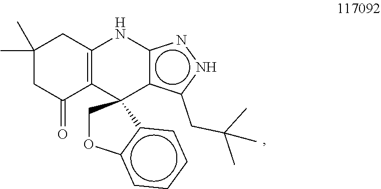

or a pharmaceutically acceptable salt, solvate, hydrate, polymorph, co-crystal, tautomer, stereoisomer, isotopically labeled compound, or prodrug thereof.

“A compound of the present disclosure” or “a compound provided herein” refers to a compound of Formula I-A, I-B, II-A, II-B, III-A, III-B, IV-A, IV-B, V-A, V-B, VI-A, or VI-B, or shown in Table 13, Table 13A, or Table 14, or a pharmaceutically acceptable salt, solvate, hydrate, polymorph, co-crystal, tautomer, stereoisomer, isotopically labeled compound, or prodrug thereof.

In some embodiments, a compound of the present disclosure is a compound of Formula I-A or I-B, or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula I-A or I-B, or a pharmaceutically acceptable salt or tautomer thereof. In some embodiments, a compound of the present disclosure is a compound of Formula I-A or I-B, or a pharmaceutically acceptable salt thereof.

In some embodiments, a compound of the present disclosure is a compound of Formula II-A or II-B, or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula II-A or II-B, or a pharmaceutically acceptable salt or tautomer thereof. In some embodiments, a compound of the present disclosure is a compound of Formula II-A or II-B, or a pharmaceutically acceptable salt thereof.

In some embodiments, a compound of the present disclosure is a compound of Formula III-A or III-B, or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula III-A or III-B, or a pharmaceutically acceptable salt or tautomer thereof. In some embodiments, a compound of the present disclosure is a compound of Formula III-A or III-B, or a pharmaceutically acceptable salt thereof.

In some embodiments, a compound of the present disclosure is a compound of Formula IV-A or IV-B, or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula IV-A or IV-B, or a pharmaceutically acceptable salt or tautomer thereof. In some embodiments, a compound of the present disclosure is a compound of Formula IV-A or IV-B, or a pharmaceutically acceptable salt thereof.

In some embodiments, a compound of the present disclosure is a compound of Formula V-A or V-B, or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula V-A or V-B, or a pharmaceutically acceptable salt or tautomer thereof. In some embodiments, a compound of the present disclosure is a compound of Formula V-A or V-B, or a pharmaceutically acceptable salt thereof.

In some embodiments, a compound of the present disclosure is a compound of Formula VI-A or VI-B, or a pharmaceutically acceptable salt, solvate, hydrate, polymorph, co-crystal, tautomer, stereoisomer, isotopically labeled compound, or prodrug thereof. In some embodiments, a compound of the present disclosure is a compound of Formula VI-A or VI-B, or a pharmaceutically acceptable salt or tautomer thereof. In some embodiments, a compound of the present disclosure is a compound of Formula VI-A or VI-B, or a pharmaceutically acceptable salt thereof.

In some embodiments, a compound of the present disclosure is a compound shown in Table 13, or a pharmaceutically acceptable salt, solvate, hydrate, polymorph, co-crystal, tautomer, stereoisomer, isotopically labeled compound, or prodrug thereof. In some embodiments, a compound of the present disclosure is a compound shown in Table 13, or a pharmaceutically acceptable salt or tautomer thereof. In some embodiments, a compound of the present disclosure is a compound shown in Table 13, or a pharmaceutically acceptable salt thereof.

In some embodiments, a compound of the present disclosure is a compound shown in Table 13A, or a pharmaceutically acceptable salt, solvate, hydrate, polymorph, co-crystal, tautomer, stereoisomer, isotopically labeled compound, or prodrug thereof. In some embodiments, a compound of the present disclosure is a compound shown in Table 13A, or a pharmaceutically acceptable salt or tautomer thereof. In some embodiments, a compound of the present disclosure is a compound shown in Table 13A, or a pharmaceutically acceptable salt thereof.

In some embodiments, a compound of the present disclosure is a compound shown in Table 14, or a pharmaceutically acceptable salt, solvate, hydrate, polymorph, co-crystal, tautomer, stereoisomer, isotopically labeled compound, or prodrug thereof. In some embodiments, a compound of the present disclosure is a compound shown in Table 14, or a pharmaceutically acceptable salt or tautomer thereof. In some embodiments, a compound of the present disclosure is a compound shown in Table 14, or a pharmaceutically acceptable salt thereof.

### Pharmaceutical Compositions, Kits, and Administration

In another aspect, the present disclosure provides pharmaceutical compositions comprising a compound provided herein and optionally a pharmaceutically acceptable excipient. In some embodiments, the pharmaceutical composition further comprises one or more additional pharmaceutical agents.

Pharmaceutical compositions can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

In some embodiments, pharmaceutical compositions are prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernal, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, in some embodiments, delayed absorption of a parenterally administered drug form is accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel. Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, May have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

In some embodiments, the compounds and compositions provided herein are administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound, mode of administration, and the like. In some embodiments, an effective amount is included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any 22 dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. In some embodiments, the amount to be administered to, for example, a child or an adolescent is determined by a medical practitioner or person skilled in the art. In some embodiments, the amount to be administered to, for example, a child or an adolescent is lower or the same as that administered to an adult.

In some embodiments, a compound or composition, as described herein, is administered in combination with one or more additional pharmaceutical agents (e.g., therapeutically and/or prophylactically active agents). In some embodiments, the compounds or compositions are administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity and/or production of a GSK3 in a subject, cell, tissue, or biological sample), improve bioavailability, improve safety, reduce drug resistance, reduce and/or modify metabolism, inhibit excretion, and/or modify distribution in a subject, cell, tissue, or biological sample. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

In some embodiments, the compound or composition is administered concurrently with, prior to, or subsequent to one or more additional pharmaceutical agents. In some embodiments, the one or more additional pharmaceutical agents are useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease. In some embodiments, each additional pharmaceutical agent is administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds or pharmaceutical compositions described herein are administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

Also encompassed by the disclosure are kits (e.g., pharmaceutical packs) comprising a compound or pharmaceutical composition provided herein; and instructions for using the compound or pharmaceutical composition provided herein. In some embodiments, the kit comprises a pharmaceutical composition or compound described herein and a container (e.g., a vial, ampule, bottle, syringe, and/or dispenser package, or other suitable container). In some embodiments, provided kits optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

In certain embodiments, the kit includes a first container comprising a compound or pharmaceutical composition provided herein. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof. In certain embodiments, the kits are useful for inhibiting the activity (e.g., aberrant activity, such as increased activity) and/or production of a GSK3 in a subject, cell, tissue, or biological sample.

In certain embodiments, a kit provided herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. In certain embodiments, the kits and instructions provide for inhibiting the activity (e.g., aberrant activity, such as increased activity) and/or production of a GSK3 in a subject, cell, or tissue. A kit described herein may include one or more additional pharmaceutical agents described herein as a separate composition.

### Methods of Treatment and Uses

Glycogen synthase kinase 3 (GSK3) remains a therapeutic target of interest for many diseases, however the development of small molecule inhibitors has been hindered by significant safety concerns related to β-catenin activation and the lack of structural information for the GSK3α paralog.

Originally identified as a protein kinase involved in the regulation of glycogen metabolism, GSK3 is now known to be a multi-functional protein with key roles in diverse biological processes including cell proliferation, differentiation, apoptosis, embryonic development, and insulin response (Cole A R, 2012; Racaud-Sultan C, Vergnolle N, 2021; Henriksen E J and Dokken B B, 2006). In addition, GSK3 is also of considerable interest as a therapeutic target because of its involvement in core pathophysiologies underlying multiple diseases including Alzheimer's Disease (AD), Fragile X Syndrome, diabetes, and several types of cancer (Bhat R V et al . . . , 2004; Beurel et al . . . , 2015; O'Leary O and Nolan Y, 2015; McCubrey J A et al . . . , 2014).

GSK3 has been of particular focus in AD because preclinical data demonstrates that modulation of this kinase has beneficial effects on both hallmark pathological processes in AD: hyperphosphorylation of tau protein and production of amyloid-beta peptides (Phiel, C J et al . . . , 2003; Plattner, F et al., 2006; Ly, P et al . . . , 2013). Based on the therapeutic potential of targeting GSK3 in AD, several GSK3 inhibitors have advanced into clinical trials (Georgievska B et al . . . , 2013; del Ser T et al . . . , 2013), but these compounds have had limited clinical success due to lack of efficacy and/or significant safety concerns. Safety concerns around GSK3 stem from evidence demonstrating a key role for this kinase in regulating the Wnt-β-catenin pathway (Behrens J et al . . . , 1998) and multiple reports have demonstrated that long-term inhibition of GSK3 can induce aberrant proliferation and hyperplasia in several tissues in vivo (Meijer et al., 2004; Sato N et al . . . , 2004; Coghlan et al . . . , 2000; Bhat R et al . . . , 2003).

GSK3 exists as two paralogs, GSK3α and GSK3β, which are encoded from separate genes and are thought to have arisen evolutionarily through gene duplication. The GSK3 paralogs are similar in sequence with 95% identity in the ATP binding site (67% amino acid identity overall) and exhibit a high degree of overlap in both tissue expression patterns and in their phosphorylation substrates (Woodgett, J R 1991; Yao H B et al., 2002; Soutar et al . . . , 2010; Kaidanovich-Beilin O, Woodgett J R 2011). Interestingly, recent studies have highlighted distinct functional roles for GSK3α in several biological processes (Beurel et al . . . , 2015). Notably, loss of GSK3β causes embryonic lethality in mice whereas loss of GSK3α results in relatively modest defects (Kim, W Y et al . . . , 2009; Morgansmith, M et al . . . , 2014). In the context of AD, most of the focus has traditionally been on GSK3β (Hooper C et al . . . , 2008). However, in a study using a mouse model that combines amyloid and tau pathologies, Hurtado et al. (2012) reported that knockdown of either GSKα or GSK3β ameliorated tau hyper-phosphorylation, yet only knockdown of GSK3α additionally reduced amyloid pathology. More recently, selective loss or inhibition of GSK3α, but not GSK3β, has been shown to rescue deficits in a mouse model of Fragile x (McCamphill P K et al . . . , 2020) and suppress tumorigenesis in models of acute myeloid leukemia (Wang Y et al . . . , 2019). With respect to safety risks, studies have shown that selective reduction or ablation of either GSK3 paralog circumvents β-catenin stabilization (Doble B W et al . . . , 2007) and that selective genetic suppression of GSKα impairs leukemia progression in mouse models of AML without increasing β-catenin levels (Banerji V et al . . . , 2012).

The challenge in discovery of selective GSK3 inhibitors is predominantly due to the high degree of homology in the ATP binding site where the primary difference is an Asp to Glu switch located in the hinge region (Wagner F F et al . . . , 2018). This difference is further complicated by the positioning of corresponding amino acid side chains located outside of the ATP binding site and therefore directed away from potential interactions with ATP competitive inhibitors. Previously a series of oxadiazole inhibitors were identified that were able to achieve ˜3-fold GSK3 selectivity for the -α paralog (Lo Monte et al., 2013) and there have also been reports of imide-based (Palomo V et al . . . , 2012) and thioxoimidazolidine kinase inhibitors that exhibit paralog selectivity up to ˜7-fold (Wang Y et al . . . , 2019). Interestingly, a series of aminopyrazole inhibitors with up to 8-fold selectivity were recently reported by Wagner et al.. (2018) and their development was based on the crystal structure of a GSK3β D133E mutant thought to mimic key differences between the two paralogs within the ATP binding site. Further results from this study also identified differential hydrogen bonding networks outside of the hinge Asp/Glu switch.

A “compound useful in a provided method” is:
a compound provided herein; or
a compound of the formula:

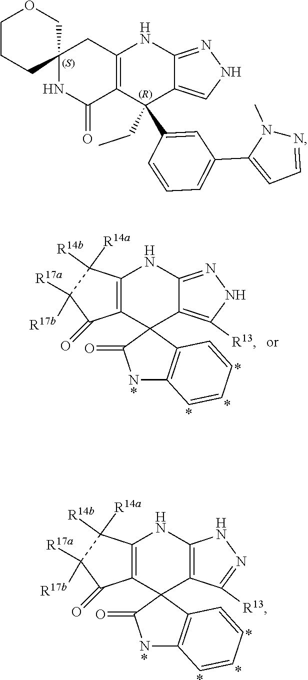

or a pharmaceutically acceptable salt, solvate, hydrate, polymorph, co-crystal, tautomer,
each instance of the atoms marked with * is independently optionally substituted;
[symbol] is a single or double bond;
R¹³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{B}, —SCN, —SR^{B}, —SSR^{B}, —N₃, —NO, —N(R^{B})₂, —NO₂, —C(═O)R^{B}, —C(═O)OR^{B}, —C(═O)SR^{B}, —C(═O)N(R^{B})₂, —C(═NR^{B})R^{B}, —C(═NR^{B})OR^{B}, —C(═NR^{B})SR^{B}, —C(═NR^{B})N(R^{B})₂, —S(═O)R^{B}, —S(═O)OR^{B}, —S(═O)SR^{B}, —S(═O)N(R^{B})₂, —S(═O)₂R^{B}, —S(═O)₂OR^{B}, —S(═O)₂SR^{B}, —S(═O)₂N(R^{B})₂, —OC(═O)R^{B}, —OC(═O)OR^{B}, —OC(═O)SR^{B}, —OC(═O)N(R^{B})₂, —OC(═NR^{B})R^{B}—OC(═NR^{B})OR^{B}, —OC(═NR^{B})SR^{B}, —OC(═NR^{B})N(R^{B})₂, —OS(═O)R^{B}, —OS(═O)OR^{B}, —OS(═O)SR^{B}, —OS(═O)N(R^{B})₂, —OS(═O)₂R^{B}, —OS(═O)₂OR^{B}, —OS(═O)₂SR^{B}, —OS(═O)₂N(R^{B})₂, —ON(R^{B})₂, —SC(═O)R^{B}, —SC(═O)OR^{B}, —SC(═O)SR^{B}, —SC(═O)N(R^{B})₂, —SC(═NR^{B})R^{B}, —SC(═NR^{B})OR^{B}, —SC(═NR^{B})SR^{B}, —SC(═NR^{B})N(R^{B})₂, —NR^{B}C(═O)R^{B}, —NR^{B}C(═O)OR^{B}, —NR^{B}C(═O)SR^{B}, —NR^{B}C(═O)N(R^{B})₂, —NR^{B}C(═NR^{B})R^{B}, —NR^{B}C(═NR^{B})OR^{B}, —NR^{B}C(═NR^{B})SR^{B}, —NR^{B}C(═NR^{B})N(R^{B})₂, —NR^{B}S(═O)R^{B}, —NR^{B}S(═O)OR^{B}, —NR^{B}S(═O)SR^{B}, —NR^{B}S(═O)N(R^{B})₂, —NR^{B}S(═O)₂R^{B}, —NR^{B}S(═O)₂OR^{B}, —NR^{B}S(═O)₂SR^{B}, —NR^{B}S(═O)₂N(R^{B})₂, —Si(R^{B})₃, —Si(R^{B})₂OR^{B}, —Si(R^{B})(OR^{B})₂, —Si(OR^{B})₃, —OSi(R^{B})₃, —OSi(R^{B})₂OR^{B}, —OSi(R^{B})(OR^{B})₂, or —OSi(OR^{B})₃;
each of R^{14a}, R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl; and
each instance of R^{B} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{B} attached to the same intervening atom are joined together with the intervening atom to form an optionally substituted, monocyclic, heterocyclic or heteroaryl ring.

In some embodiments, [symbol] is a single bond. In some embodiments, [symbol] is a double bond.

In some embodiments, R¹³ is halogen, optionally substituted alkyl, optionally substituted alkenyl, optionally substituted alkynyl, optionally substituted heteroalkyl, optionally substituted heteroalkenyl, optionally substituted heteroalkynyl, —OR^{B}, —SR^{B}, or —N(R^{B})₂. In some embodiments, R¹³ is optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, or optionally substituted C₁₋₁₀ heteroalkynyl. In some embodiments, R¹³ is Cie haloalkyl. In some embodiments, R¹³ is C₁₋₄ haloalkyl. In some embodiments, R¹³ is C₁₋₄ fluoroalkyl (e.g., C₁₋₄ perfluoroalkyl). In some embodiments, R¹³ is —CF₃. In some embodiments, R¹³ is hydrogen, optionally substituted C₁-C₆ alkyl, or halogen. In some embodiments, R¹³ is hydrogen, fluorine, —CH₃, —CH₂F, —CHF₂, or —CF₃. In some embodiments, R¹³ is hydrogen, fluorine, —CH₃, or —CF₃. In some embodiments, R¹³ is hydrogen or halogen. In some embodiments, R¹³ is hydrogen or fluorine. In some embodiments, R¹³ is hydrogen or optionally substituted C₁-C₆ alkyl. In some embodiments, R¹³ is hydrogen, unsubstituted C₁-C₆ alkyl, or C₁₋₆ haloalkyl. In some embodiments, R¹³ is hydrogen.

In some embodiments, R¹³ is halogen. In some embodiments, R¹³ is bromine, chlorine, or fluorine. In some embodiments, R¹³ is bromine or chlorine. In some embodiments, R¹³ is chlorine or fluorine. In some embodiments, R¹³ is bromine. In some embodiments, R¹³ is chlorine. In some embodiments, R¹³ is fluorine.

In some embodiments, R¹³ is —OR^{B}, —SR^{B}, or —N(R^{B})₂ (e.g., wherein R^{B} is hydrogen or optionally substituted alkyl, or optionally substituted phenyl). In some embodiments, R¹³ is —OH. In some embodiments, R¹³ is —SH. In some embodiments, R¹³ is —NH₂. In some embodiments, R¹³ is —CN, —SCN, —SSR^{B}, —N₃, —NO, or —NO₂. In some embodiments, R¹³ is —C(═O)R^{B}, —C(═O)OR^{B}, —C(═O)SR^{B}, or —C(═O)N(R^{B})₂ (e.g., wherein R^{B} is hydrogen or optionally substituted alkyl, or optionally substituted phenyl). In some embodiments, R¹³ is —C(═NR^{B})R^{B}, —C(═NR^{B})OR^{B}, —C(═NR^{B})SR^{B}, or —C(═NR^{B})N(R^{B})₂ (e.g., wherein R^{B} is hydrogen or optionally substituted alkyl, or optionally substituted phenyl). In some embodiments, R¹³ is —S(═O)R^{B}, —S(═O)OR^{B}, —S(═O)SR^{B}, —S(═O)N(R^{B})₂, —S(═O)₂R^{B}, —S(═O)₂OR^{B}, —S(═O)₂SR^{B}, or —S(═O)₂N(R^{B})₂ (e.g., wherein R^{B} is hydrogen or optionally substituted alkyl, or optionally substituted phenyl). In some embodiments, R¹³ is —OC(═O)R^{B}, —OC(═O)OR^{B}, —OC(═O)SR^{B}, —OC(═O)N(R^{B})₂, —OC(═NR^{B})R^{B}, —OC(═NR^{B})OR^{B}, —OC(═NR^{B})SR^{B}, —OC(═NR^{B})N(R^{B})₂, —OS(═O)R^{B}, —OS(═O)OR^{B}, —OS(═O)SR^{B}, —OS(═O)N(R^{B})₂, —OS(═O)₂R^{B}, —OS(═O)₂OR^{B}, —OS(═O)₂SR^{B}, —OS(═O)₂N(R^{B})₂, or —ON(R^{B})₂ (e.g., wherein R^{B} is hydrogen or optionally substituted alkyl, or optionally substituted phenyl). In some embodiments, R¹³ is —SC(═O)R^{B}, —SC(═O)OR^{B}, —SC(═O)SR^{B}, —SC(═O)N(R^{B}))₂, —SC(═NR^{B})R^{B}, —SC(═NR^{B})OR^{B}, —SC(═NR^{B})SR^{B}, or —SC(═NR^{B})N(R^{B})₂ (e.g., wherein R^{B} is hydrogen or optionally substituted alkyl, or optionally substituted phenyl). In some embodiments, R¹³ is —NR^{B}C(═O)R^{B}, —NR^{B}C(═O)OR^{B}, —NR^{B}C(═O)SR^{B}, —NR^{B}C(═O)N(R^{B})₂, —NR^{B}C(═NR^{B})R^{B}, —NR^{B}C(═NR^{B})OR^{B}, —NR^{B}C(═NR^{B})SR^{B}, —NR^{B}C(═NR^{B})N(R^{B})₂, —NR^{B}S(═O)R^{B}, —NR^{B}S(═O)OR^{B}, —NR^{B}S(═O)SR^{B}, —NR^{B}S(═O)N(R^{B})₂, —NR^{B}S(═O)₂R^{B}, —NR^{B}S(═O)₂OR^{B}, —NR^{B}S(═O)₂SR^{B}, or —NR^{B}S(═O)₂N(R^{B})₂ (e.g., wherein R^{B} is hydrogen or optionally substituted alkyl, or optionally substituted phenyl). In some embodiments, R¹³ is —Si(R^{B})₃, —Si(R^{B})₂OR^{B}, —Si(R^{B})(OR^{B})₂, —Si(OR^{B})₃, —OSi(R^{B})₃, —OSi(R^{B})₂OR^{B}, —OSi(R^{B})(OR^{B})₂, or —OSi(OR^{B})₃ (e.g., wherein R^{B} is hydrogen or optionally substituted alkyl, or optionally substituted phenyl).

In some embodiments, R¹³ is optionally substituted carbocyclyl, optionally substituted heterocyclyl, optionally substituted aryl, or optionally substituted heteroaryl. In some embodiments, R¹³ is optionally substituted C₃₋₁₄ carbocyclyl. In some embodiments, R¹³ is optionally substituted monocyclic C₃₋₇ carbocyclyl. In some embodiments, R¹³ is optionally substituted monocyclic C₃₋₄ carbocyclyl. In some embodiments, R¹³ is optionally substituted monocyclic C₅₋₇ carbocyclyl. In some embodiments, R¹³ is saturated carbocyclyl. In some embodiments, R¹³ is carbocyclyl comprising only one unsaturated bond (e.g., C═C bond) in the carbocyclic ring system.

In some embodiments, R¹³ is optionally substituted 3- to 14-membered heterocyclyl. In some embodiments, R¹³ is optionally substituted monocyclic 3- to 7-membered heterocyclyl. In some embodiments, R¹³ is optionally substituted 3- to 14-membered heterocyclyl comprising one or more O and/or S atoms but no N atoms. In some embodiments, R¹³ is optionally substituted 3- to 14-membered heterocyclyl comprising one or more N atoms and optionally one or more O and/S atoms. In some embodiments, R¹³ is optionally substituted monocyclic 3- to 7-membered heterocyclyl comprising one or more O and/or S atoms but no N atoms. In some embodiments, R¹³ is optionally substituted monocyclic 3- to 7-membered heterocyclyl comprising one or more N atoms and optionally one or more O and/or S atoms. In some embodiments, R¹³ is saturated heterocyclyl. In some embodiments, R¹³ is heterocyclyl comprising only one unsaturated bond (e.g., C═C bond) in the heterocyclic ring system.

In some embodiments, R¹³ is optionally substituted monocyclic aryl. In some embodiments, R¹³ is optionally substituted bicyclic aryl. In some embodiments, R¹³ is optionally substituted C₆₋₁₄ aryl. In some embodiments, R¹³ is optionally substituted C₆₋₁₀ aryl. In some embodiments, R¹³ is optionally substituted phenyl. In some embodiments, R¹³ is optionally substituted naphthyl.

In some embodiments, R¹³ is optionally substituted monocyclic heteroaryl. In some embodiments, R¹³ is optionally substituted bicyclic heteroaryl. In some embodiments, R¹³ is optionally substituted 5- to 14-membered heteroaryl. In some embodiments, R¹³ is optionally substituted 5- to 10-membered heteroaryl. In some embodiments, R¹³ is optionally substituted 5- to 6-membered monocyclic heteroaryl.

In some embodiments, at least one instance of R^{B} is independently hydrogen, optionally substituted alkyl, optionally substituted alkenyl, optionally substituted alkynyl, optionally substituted heteroalkyl, optionally substituted heteroalkenyl, or optionally substituted heteroalkynyl. In some embodiments, at least one instance of R^{B} is optionally substituted carbocyclyl, optionally substituted heterocyclyl, optionally substituted aryl, or optionally substituted heteroaryl. In some embodiments, at least one instance of R^{B} is independently hydrogen, optionally substituted C₁₋₁₀ alkyl, optionally substituted C₁₋₁₀ alkenyl, optionally substituted C₁₋₁₀ alkynyl, optionally substituted C₃₋₁₄ carbocyclyl, or optionally substituted C₆₋₁₄ aryl. In some embodiments, at least one instance of R^{B} is independently hydrogen, optionally substituted C₁₋₁₀ alkyl, or optionally substituted phenyl. In some embodiments, at least one instance of R^{B} is a nitrogen protecting group when attached to a nitrogen atom, an oxygen protecting group when attached to an oxygen atom, or a sulfur protecting group when attached to a sulfur atom. In some embodiments, two instances of R^{E} attached to the same intervening atom are joined together with the intervening atom to form an optionally substituted, monocyclic, heterocyclic or heteroaryl ring.

In some embodiments, each of R^{14a}, R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl. In some embodiments, each of R^{14a}, R^{14b}, R^{17a}, and R^{17b} is independently hydrogen, halogen, or optionally substituted alkyl. In some embodiments, each of R^{14a}, R^{14b}, R^{17a}, and R^{17b} is independently hydrogen, halogen, or optionally substituted C₁₋₄ alkyl.

In some embodiments, each of R^{14a} and R^{14b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, each of R^{14a} and R^{14b} is independently hydrogen, halogen, optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted Cujo heteroalkenyl, optionally substituted C₁₋₁₀ heteroalkynyl, optionally substituted C₃₋₁₄ carbocyclyl, optionally substituted 3- to 14-membered heterocyclyl, optionally substituted C₆₋₁₄ aryl, or optionally substituted 5- to 14-membered heteroaryl. In some embodiments, at least one of R^{14a} and R^{14b} is hydrogen.

In some embodiments, each of R^{17a} and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl. In some embodiments, each of R^{17a} and R^{17b} is independently hydrogen, halogen, optionally substituted C₁₋₁₀ alkyl, optionally substituted C₁₋₁₀ alkenyl, optionally substituted C₁₋₁₀ alkynyl, optionally substituted C₁₋₁₀ heteroalkyl, optionally substituted C₁₋₁₀ heteroalkenyl, optionally substituted C₁₋₁₀ heteroalkynyl, optionally substituted C₃₋₁₄ carbocyclyl, optionally substituted 3- to 14-membered heterocyclyl, optionally substituted C₆₋₁₄ aryl, or optionally substituted 5- to 14-membered heteroaryl. In some embodiments, at least one of R^{17a} and R^{17b} is hydrogen.

Compounds useful in a provided method and pharmaceutical compositions provided herein may be useful for the inhibition of a GSK3. Without being bound by any particular theory, the compounds useful in a provided method and the pharmaceutical compositions provided herein being useful as described herein may be at least in part due to their inhibition of the activity and/or production of a GSK3. Compared to known GSK3 inhibitors, the compounds useful in a provided method and the pharmaceutical compositions provided herein may increase the potency, efficacy, and/or selectivity in inhibiting the activity and/or production of a GSK3 in a subject, cell, or tissue. Compared to known GSK3 inhibitors, the compounds useful in a provided method and the pharmaceutical compositions provided herein may increase bioavailability, safety, and/or therapeutic window, reduce toxicity and/or resistance, and/or increase subject compliance, in a subject. In certain embodiments, the compounds useful in a provided method are selective GSK3 inhibitors (e.g., GSK3 inhibitors that selectively inhibit a GSK3 over one or more other kinases). In certain embodiments, the compounds useful in a provided method are selective GSK3α inhibitors (e.g., GSK3α inhibitors that selectively inhibit GSK3α over GSK3β and optionally one or more other kinases). In certain embodiments, the compounds useful in a provided method are selective GSK3 inhibitors (e.g., GSK3ß inhibitors that selectively inhibit GSK3β over GSK3α and optionally one or more other kinases).

In some embodiments, the GSK3 is glycogen synthase kinase 3 α (GSK3α). In some embodiments, the compound provided herein is more selective for inhibiting the activity and/or production of GSK3α than glycogen synthase kinase 3 β (GSK3β) in an in vitro assay. In some embodiments, the selectivity of the compound provided herein for GSK3α over GSK3β is determined by the relative K_{d} values for GSK3α and GSKβ. In certain embodiments, the selectivity of the compound provided herein for GSK3α over GSK3β is determined by the quotient of the IC₅₀ value of the compound in inhibiting GSK38 over the IC₅₀ value of the compound in inhibiting GSK3α. In certain embodiments, the selectivity of the compound provided herein for GSK3α over GSK3β is at least 1.1, at least 1.3, at least 1.5, at least 1.7, at least 2, at least 3, at least 4, at least 6, at least 8, at least 10, at least 12, at least 15, at least 17, at least 20, at least 30, at least 50, at least 100, at least 1,000, or at least 10,000. In certain embodiments, the selectivity of the compound provided herein for GSK3α over GSK3β is at least 8. In certain embodiments, the selectivity of the compound provided herein for GSK3α over GSK3β is at least 10. In certain embodiments, the selectivity of the compound provided herein for GSK3α over GSK3β is at least 12. In certain embodiments, the selectivity of the compound provided herein for GSK3α over GSK3β is at least 15. In certain embodiments, the selectivity of the compound provided herein for GSK3α over GSK3β is at least 17. In certain embodiments, the selectivity of the compound provided herein for GSK30 over GSK3ß is at least 20. In certain embodiments, the selectivity of the compound provided herein for GSK3α over GSK3β is at least 30. In certain embodiments, the selectivity of the compound provided herein for GSK3α over GSK3β is at least 50. In certain embodiments, the selectivity of the compound provided herein for GSK3α over GSK3β is at least 100. In certain embodiments, the selectivity of the compound provided herein for GSK3α over GSK3β is between 10 and 1,000, inclusive.

In some embodiments, the GSK3 is glycogen synthase kinase 3 β (GSK3β). In some embodiments, the compound provided herein is more selective for inhibiting the activity and/or production of GSK3β than glycogen synthase kinase 3 α (GSK3α) in an in vitro assay. In some embodiments, the selectivity of the compound provided herein for GSK3β over GSK3α is determined by the relative K_{d} values for GSK3β and GSKα. In certain embodiments, the selectivity of the compound provided herein for GSK3β over GSK3α is determined by the quotient of the IC₅₀ value of the compound in inhibiting GSK3α over the IC₅₀ value of the compound in inhibiting GSK3β. In certain embodiments, the selectivity of the compound provided herein for GSK3β over GSK3α is at least 1.1, at least 1.3, at least 1.5, at least 1.7, at least 2, at least 3, at least 4, at least 6, at least 8, at least 10, at least 12, at least 15, at least 17, at least 20, at least 30, at least 50, at least 100, at least 1,000, or at least 10,000. In certain embodiments, the selectivity of the compound provided herein for GSK3ß over GSK3α is at least 8. In certain embodiments, the selectivity of the compound provided herein for GSK3β over GSK3α is at least 10. In certain embodiments, the selectivity of the compound provided herein for GSK3β over GSK3α is at least 12. In certain embodiments, the selectivity of the compound provided herein for GSK3β over GSK3α is at least 15. In certain embodiments, the selectivity of the compound provided herein for GSK3β over GSK3α is at least 17. In certain embodiments, the selectivity of the compound provided herein for GSK3β over GSK3α is at least 20. In certain embodiments, the selectivity of the compound provided herein for GSK3β over GSK3α is at least 30. In certain embodiments, the selectivity of the compound provided herein for GSK3β over GSK3α is at least 50. In certain embodiments, the selectivity of the compound provided herein for GSK3β over GSK3α is at least 100. In certain embodiments, the selectivity of the compound provided herein for GSK3ß over GSK3α is between 10 and 1,000, inclusive.

In another aspect, provided herein is a method of treating a disease in a subject in need thereof, the method comprising administering to the subject an effective amount of: a compound useful in a provided method or a pharmaceutical composition provided herein. In another aspect, provided herein is a use a compound useful in a provided method or pharmaceutical composition provided herein for the manufacture of a medicament for treating a disease in a subject in need thereof. In another aspect, provided herein is a compound useful in a provided method or pharmaceutical composition provided herein for use in treating a disease in a subject in need thereof.

In another aspect, provided herein is a method of preventing a disease in a subject in need thereof, the method comprising administering to the subject an effective amount of: a compound useful in a provided method or a pharmaceutical composition provided herein. In another aspect, provided herein is a use of a compound useful in a provided method or pharmaceutical composition provided herein for the manufacture of a medicament for preventing a disease in a subject in need thereof. In another aspect, provided herein is a compound useful in a provided method or pharmaceutical composition provided herein for use in preventing a disease in a subject in need thereof.

In some embodiments the effective amount is further effective in inhibiting the activity and/or production of a GSK3. In some embodiments, the effective amount is a therapeutically effective amount. In some embodiments, the effective amount is a prophylactically effective amount. In certain embodiments, the effective amount is an amount effective for treating a disease in a subject in need thereof. In certain embodiments, the effective amount is an amount effective for preventing a disease in a subject in need thereof. In certain embodiments, the effective amount is an amount effective for reducing the risk of developing a disease in a subject in need thereof. In certain embodiments, the effective amount is an amount effective for inhibiting the activity (e.g., aberrant activity, such as increased activity) of a GSK3 in a subject, cell, tissue, or biological sample. In certain embodiments, the effective amount is an amount effective for inhibiting the production of a GSK3 in a subject, cell, tissue, or biological sample.

In certain embodiments, the effective amount is an amount effective for inhibiting the activity of a GSK3 by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of a GSK3 by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting the activity of a GSK3 by a range between a percentage described in this paragraph and another percentage described in this paragraph, inclusive.

In some embodiments, the term refers to a reduction of the level of production, e.g., GSK3 protein production, to a level that is statistically significantly lower than an initial level, which may, for example, be a baseline level of production. In some embodiments, the term refers to a reduction of the level of production, e.g., GSK3 protein production, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of production.

In some embodiments, the disease is associated with aberrantly high activity and/or production of catenin beta-1. In some embodiments, the disease is associated with a mutation and/or overexpression of the CTNNB1 gene. In some embodiments, the disease is a mental or behavioral disease. In some embodiments, the disease is fragile X syndrome. In some embodiments, the disease is autism. In some embodiments, the disease is schizophrenia. In some embodiments, the disease is bipolar disorder. In some embodiments, the disease is attention deficit hyperactivity disorder. In some embodiments, the disease is a neurological disease. In some embodiments, the disease is seizure. In some embodiments, the disease is Alzheimer's disease. In some embodiments, the disease is Huntington's disease. In some embodiments, the disease is Parkinson's disease. In some embodiments, the disease is amyotrophic lateral sclerosis. In some embodiments, the disease is a cancer. In some embodiments, the disease is a hematological malignancy. In some embodiments, the disease is leukemia. In some embodiments, the disease is acute myeloid leukemia. In some embodiments, the disease is acute lymphoblastic leukemia. In some embodiments, the disease is colon cancer. In some embodiments, the disease is pancreatic cancer. In some embodiments, the disease is a metabolic disease. in some embodiments, the disease is diabetes. In some embodiments, the disease is Type II diabetes. In some embodiments, the disease is obesity.

In another aspect, provided herein is a method of inhibiting the activity and/or production of a glycogen synthase kinase 3 (GSK3) in a subject in need thereof, the method comprising administering to the subject an effective amount of: a compound useful in a provided method or a pharmaceutical composition provided herein.

In another aspect, provided herein is a method of inhibiting the activity and/or production of a glycogen synthase kinase 3 (GSK3) in a cell, tissue, or biological sample, the method comprising contacting the cell, tissue, or biological sample with an effective amount of: a compound useful in a provided method or a pharmaceutical composition provided herein.

In certain embodiments, the cell, tissue, or biological sample is in vitro. In certain embodiments, the cell, tissue, or biological sample is in vivo.

In certain embodiments, the subject is an animal. In certain embodiments, the subject is a human. In certain embodiments, the subject is a human aged 18 years and older. In some embodiments, the subject is a human aged <2 years. In some embodiments, the subject is a human aged 2-6 years, inclusive. In some embodiments, the subject is a human aged 6-18 years, inclusive. In some embodiments, the subject is a human aged 18-65 years, inclusive. In some embodiments, the subject is a human aged >65 years. In certain embodiments, the subject is a non-human animal. In certain embodiments, the subject is a mammal. In certain embodiments, the subject is a non-human mammal. In some embodiments, the subject is a research animal.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### General Methods of Preparing the Compounds Useful in a Provided Method

### Example 1. Preparation of Vinylogous Amides

### Protocol A:

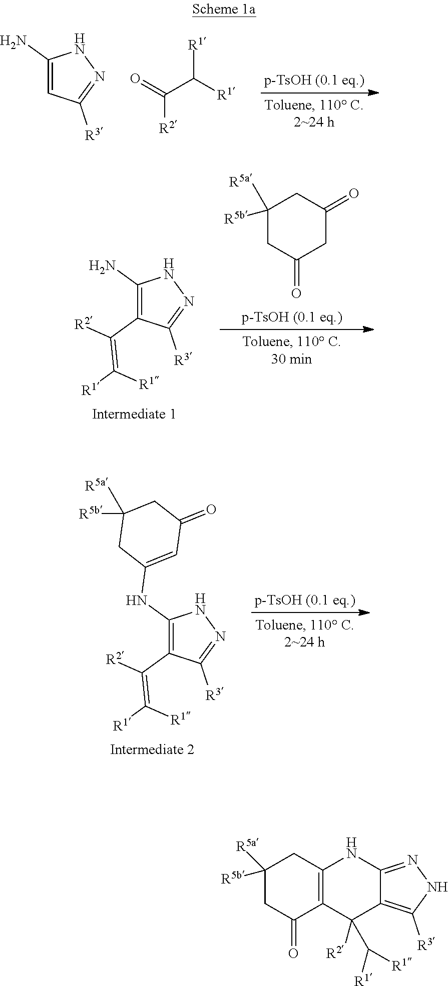

In Synthesis Protocol A (Scheme 1a), ketone (1.0 equivalent) and amine (1.0 equivalent) were dissolved in toluene (0.1-0.5 M). p-Toluenesulfonic acid (0.1 equivalent) was added, and the mixture was heated at 110° C. The reaction mixture was cooled, and toluene was evaporated. The crude reaction mixture was purified by column chromatography on silica to afford intermediate 1.

The resulting intermediate 1 (1.0 equivalent) was then dissolved in toluene and 1,3-diketone (1.0 equivalent) followed by p-toluenesulfonic acid (0.1 equivalent) were added. The mixture was heated at 110° C. for 30 minutes to afford the uncyclized intermediate 2 quantitatively. Toluene was evaporated to remove the water generated in the enamine formation reaction.

Fresh toluene was added and the mixture was heated at 110° C. After complete conversion of the uncyclized intermediate 2, toluene was evaporated and the crude mixture was purified by column chromatography on silica to afford the cyclized product.

### Protocol B:

In Synthesis Protocol B, the uncyclized intermediate 2 was dissolved in toluene, and trifluoroacetic acid (1.0 equivalent) was added. The mixture was heated at 110° C. After complete conversion of the uncyclized intermediate, volatiles were evaporated, and the crude mixture was purified by column chromatography on silica to afford the cyclized product.

### Protocol C:

In Synthesis Protocol C, the uncyclized intermediate 2 was dissolved in trifluoroacetic acid (0.5M), and the mixture was heated at 73° C. After complete conversion of the uncyclized intermediate, volatiles were evaporated, and the crude mixture was purified by column chromatography on silica to afford the cyclized product.

### Protocol D:

In Synthesis Protocol D, the uncyclized intermediate 2 was dissolved in trifluoroacetic acid (0.5M), and the mixture was heated at 140° C. in the microwave. After complete conversion of the uncyclized intermediate, volatiles were evaporated, and the crude mixture was purified by column chromatography on silica to afford the cyclized product.

### Example 2. Preparation of Compound 913006

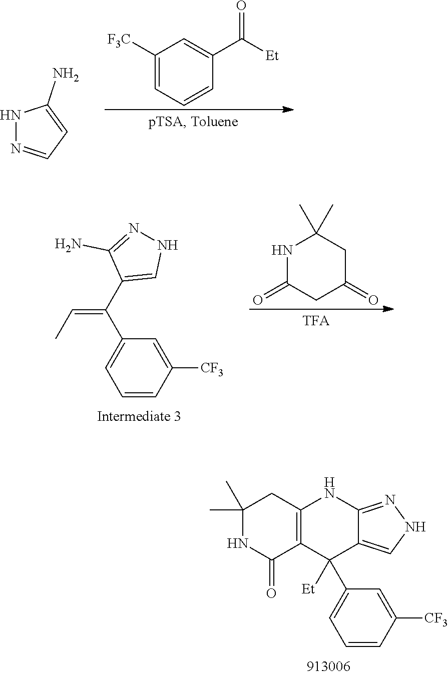

### (E)-4-(1-(3-(trifluoromethyl)phenyl) prop-1-en-1-yl)-1H-pyrazol-3-amine (Intermediate 3)

1-(3-(trifluoromethyl)phenyl) propan-1-one (25.0 g, 1 Eq. 123.7 mmol) and 1H-pyrazol-3-amine (10.3 g, 1 Eq, 124 mmol) were suspended in toluene (400 mL). p-Toluenesulfonic acid monohydrate (2.35 g, 0.1 Eq. 12.4 mmol) was added, and the mixture was stirred for two days under Dean-Stark conditions. After ¹H-NMR showed full conversion, the mixture was cooled to room temperature and concentrated in vacuo to give 36.8 g (quantitative) of a brown oil (Intermediate 3) that was used as such in the next step. 4-ethyl-7,7-dimethyl-4-(3-(trifluoromethyl)phenyl)-2,4,6,7,8,9-hexahydro-5H-pyrazolo[3,4-b][1,6]naphthyridin-5-one (TBME-adduct) (913006)

The reaction was carried out in 4 identical batches, which were combined after the reaction.

(E)-4-(1-(3-(trifluoromethyl)phenyl) prop-1-en-1-yl)-1H-pyrazol-3-amine (Intermediate 3) (33.1 g, 1 Eq, 124 mmol) and 6,6-dimethylpiperidine-2,4-dione (17.5 g, 1 Eq, 124 mmol) were dissolved in TFA (254 g, 172 mL, 18 Eq, 2.23 mol). The mixture was stirred at 110° C. in a pressure tube in a sand bath for 4 days.

After ¹H-NMR showed almost full conversion towards the product, the mixture was cooled to room temperature. The mixture was concentrated in vacuo. The mixture was basified with 2N NaOH (aq.) The formed solids were filtered off and washed with H₂O and TBME and dried in vacuo. The product was obtained as a light yellow solid (10.6 g). The filtrate was concentrated in vacuo. The solids were filtered off and washed with TBME to give a second crop (5.73 g). The two crops were combined and dried in vacuo to give 14.9 g (31%) of 3.

### Example 3. Preparation of Compound 826648

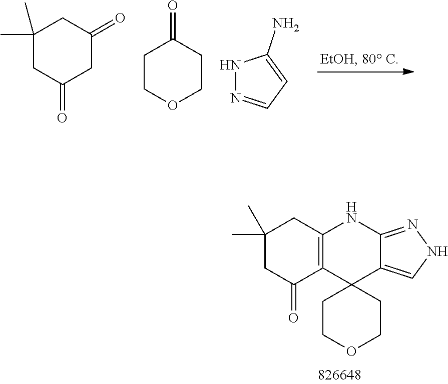

7′,7′-dimethyl-2,2′,3,5,6,7′,8′,9′-octahydrospiro[pyran-4,4′-pyrazolo[3,4-b]quinolin]-5′ (6′H)-one (826648). A solution of 5,5-dimethylcyclohexane-1,3-dione (506 mg, 3.61 mmol, 1 eq.), tetrahydro-4H-pyran-4-one (361 mg, 3.61 mmol, 1 eq.), and 1H-pyrazol-3-amine (300 mg, 3.61 mmol, 1 eq.) in ethanol (5.0 mL) was stirred at 80° C. for 18 h in a sealed tube. A suspension was formed, and the reaction mixture was filtered. The collected filter cake was washed with EtOAc (2 mL) and PE (20 mL) to provide 826648 (322 mg, 31% yield) as a white solid. LCMS: 288.0 [M+H]⁺.

### Example 4. Preparation of Fluorinated Compounds

Intermediates were synthesized according to the following scheme.

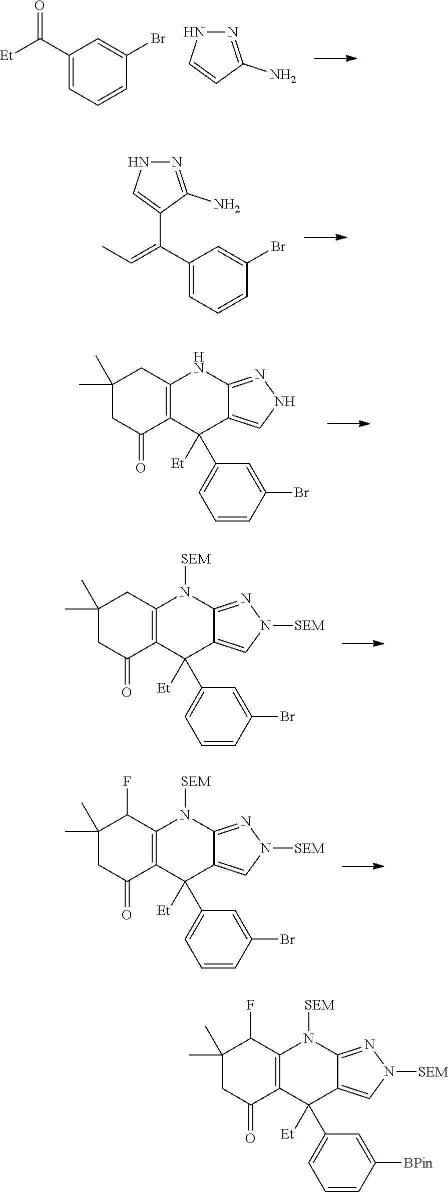

### Preparation of 4-(1-(3-bromophenyl) prop-1-en-1-yl)-1H-pyrazol-3-amine (mixture of E/Z isomers)

1H-pyrazol-3-amine (3.50 g, 42.1 mmol), 1-(3-bromophenyl) propan-1-one (8.98 g, 42.1 mmol) and 4-methylbenzenesulfonic acid hydrate (801 mg, 4.21 mmol) were combined then toluene (50.0 mL) was added. The mixture was stirred at 125° C. for 18 hours under N₂ atmosphere. The mixture was then concentrated and the residue was purified by flash chromatography (CH₂Cl₂:MeOH=100:0 to 99:1 to 98:2) to supply 4-(1-(3-bromophenyl) prop-1-en-1-yl)-1H-pyrazol-3-amine (10.0 g, 35.95 mmol, 85% yield, 98% purity) as a mixture of isomers and as a pale-yellow solid. LCMS m/z=279.9 (M+H)+.

### Preparation of 4-(3-bromophenyl)-4-ethyl-7,7-dimethyl-2,4,6,7,8,9-hexahydro-5H-pyrazolo[3,4-b]quinolin-5-one

4-(1-(3-bromophenyl) prop-1-en-1-yl)-1H-pyrazol-3-amine (10.0 g, 35.95 mmol) and 5,5-dimethylcyclohexane-1,3-dione (5.04 g, 35.95 mmol) were dissolved in trifluoroethanol (71.9 mL) and TFA (4.10 g, 35.95 mmol, 2.75 mL) was then added. The mixture was split into four microwave vials. The vials were then heated to 150° C. in a microwave for 1.5 hours then cooled to room temperature. The samples were combined and the mixture was concentrated and purified by column chromatography (5-50% EtOAc/EtOH:Heptanes) to provide 4-(3-bromophenyl)-4-ethyl-7,7-dimethyl-2,4,6,7,8,9-hexahydro-5H-pyrazolo[3,4-b]quinolin-5-one (4.11 g, 10.27 mmol, 29% yield). LCMS m/z=402.1 (M+H)+.

### Preparation of 4-(3-bromophenyl)-4-ethyl-7,7-dimethyl-2,9-bis((2-(trimethylsilyl) ethoxy)methyl)-2,4,6,7,8,9-hexahydro-5H-pyrazolo[3,4-b]quinolin-5-one

A solution of 4-(3-bromophenyl)-4-ethyl-7,7-dimethyl-2,4,6,7,8,9-hexahydro-5H-pyrazolo[3,4-b]quinolin-5-one (4.40 g, 10.99 mmol) in THF (110 mL) was cooled to 0° C. and sodium hydride (923 mg, 38.5 mmol, 60% in mineral oil) was added portionwise. After the addition, the mixture was stirred at 0° C. for 1 hour. SEM-chloride (7.33 g, 43.96 mmol, 7.80 mL) was then added dropwise and the resulting mixture was stirred at 0° C. for 2 hour then warmed to room temperature and stirred overnight. The mixture was poured into 50 mL of ice-water slowly, then extracted with EtOAc (30 mL×2). The combined organic layers were dried over MgSO₄ and concentrated. The residue was purified by column chromatography (5-20% EtOAc in heptane) to supply 4-(3-bromophenyl)-4-ethyl-7,7-dimethyl-2,9-bis((2-(trimethylsilyl) ethoxy)methyl)-2,4,6,7,8,9-hexahydro-5H-pyrazolo[3,4-b]quinolin-5-one (5.40 g, 8.17 mmol, 74% yield) as pale-yellow gum. LCMS m/z=662.3 (M+H)+.

### Preparation of 4-(3-bromophenyl)-4-ethyl-8-fluoro-7,7-dimethyl-2,9-bis((2-(trimethylsilyl) ethoxy)methyl)-2,4,6,7,8,9-hexahydro-5H-pyrazolo[3,4-b]quinolin-5-one

4-(3-bromophenyl)-4-ethyl-7,7-dimethyl-2,9-bis((2-(trimethylsilyl) ethoxy)methyl)-2,4,6,7,8,9-hexahydro-5H-pyrazolo[3,4-b]quinolin-5-one (1.52 g, 2.30 mmol) was dissolved in THF (23.0 mL) and cooled in a dry ice-acetone bath to −78° C. [bis(trimethylsilyl)amino]lithium (1 M in toluene, 2.19 mL) was then added and the mixture was stirred for 30 minutes then warmed to 0° C. and stirred for an additional 30 minutes. The mixture was then cooled to −78° C. and NFSI (798 mg, 2.53 mmol) was added. The mixture was allowed to warm to room temperature overnight. The mixture was then quenched upon addition of water and saturated, aqueous NH₄Cl. The mixture was then extracted with ethyl acetate (50 mL×3) and the combined organic layers were dried over sodium sulfate, filtered, and concentrated. The crude material was then purified by column chromatography (5 to 25% EtOAc in heptanes) to provide 4-(3-bromophenyl)-4-ethyl-8-fluoro-7,7-dimethyl-2,9-bis((2-(trimethylsilyl) ethoxy)methyl)-2,4,6,7,8,9-hexahydro-5H-pyrazolo[3,4-b]quinolin-5-one (1.15 g, 1.69 mmol, 74% yield) as a mixture of diastereomers. LCMS m/z=680.7 (M+H)+.

### Preparation of 4-ethyl-8-fluoro-7,7-dimethyl-4-(3-(4,4,5,5-tetramethyl-1,3,2-dioxaborolan-2-yl)phenyl)-2,9-bis((2-(trimethylsilyl) ethoxy)methyl)-2,4,6,7,8,9-hexahydro-SH-pyrazolo[3,4-b]quinolin-5-one

4-(3-bromophenyl)-4-ethyl-8-fluoro-7,7-dimethyl-2,9-bis((2-(trimethylsilyl) ethoxy)methyl)-2,4,6,7,8,9-hexahydro-5H-pyrazolo[3,4-b]quinolin-5-one (297 mg, 437 μmol), 4,4,5,5-tetramethyl-2-(4,4,5,5-tetramethyl-1,3,2-dioxaborolan-2-yl)-1,3,2-dioxaborolane (144 mg, 568 μmol), Pd (dppf) Cl₂—CH₂Cl₂ (17.9 mg, 21.85 μmol) and KOAc (85.8 mg, 874 μmol) were combined in a vial and dissolved in dioxane (2.19 mL). Nitrogen was bubbled through the solution for 5 min then the vial was sealed and stirred at 100° C. for 18 hours. The mixture was then concentrated and dissolved in EtOAc. The mixture was filtered and the filtrate was concentrated. The residue was purified by column chromatography (5 to 60% EtOAc in heptanes) to afford 4-ethyl-8-fluoro-7,7-dimethyl-4-(3-(4,4,5,5-tetramethyl-1,3,2-dioxaborolan-2-yl)phenyl)-2,9-bis((2-(trimethylsilyl) ethoxy)methyl)-2,4,6,7,8,9-hexahydro-5H-pyrazolo[3,4-b]quinolin-5-one (231 mg, 318 μmol, 73% yield) as an orange oil. LCMS m/z=726.9 (M+H)+.

### Preparation of 4-(3-(1,3-dimethyl-1H-pyrazol-4-yl)phenyl)-4-ethyl-8-fluoro-7,7-dimethyl-2,4,6,7,8,9-hexahydro-5H-pyrazolo[3,4-b]quinolin-5-one

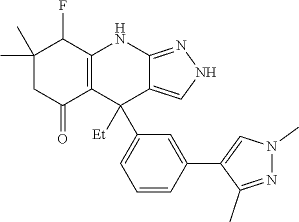

4-(3-bromophenyl)-4-ethyl-8-fluoro-7,7-dimethyl-2,9-bis((2-(trimethylsilyl) ethoxy)methyl)-2,4,6,7,8,9-hexahydro-5H-pyrazolo[3,4-b]quinolin-5-one (200 mg, 295 μmol), 1,3-dimethyl-4-(4,4,5,5-tetramethyl-1,3,2-dioxaborolan-2-yl) pyrazole (131 mg, 589 μmol), [2-(2-aminophenyl)phenyl]-methylsulfonyloxy-palladium; dicyclohexyl-[2-(2,4,6-triisopropylphenyl)phenyl]phosphane (12.5 mg, 14.7 μmol), and potassium phosphate tribasic (125 mg, 589 μmol) were added to a reaction vial. The vial was then sealed and degassed by evacuating and backfilling with nitrogen. Dioxane (786 uL) and water (393 uL) were then added and the mixture was stirred at 100° C. for 24 hours. The mixture was then cooled to room temperature, concentrated, and used directly in the next step.

The crude mixture of 4-[3-(1,3-dimethylpyrazol-4-yl)phenyl]-4-ethyl-7,7-dimethyl-2,9-bis(2-trimethylsilylethoxymethyl)-6,8-dihydropyrazolo[3,4-b]quinolin-5-one (204.5 mg, 302.5 μmol) was dissolved in TFA (1.51 mL) and CH₂Cl₂ (4.54 mL) and stirred at room temperature for 3 hours under nitrogen. The mixture was then concentrated and purified by column chromatography (5-100% [3:1 EtOAc:EtOH] in heptanes). The residue obtained was then purified by chiral SFC (CHIRALPAK IB 30×250 mm, 5 um, Method: 30% MeOH w/0.1% DEA in CO₂ [flow rate: 100 mL/min, ABPR 120bar, MBPR 40 psi, column temp 40 deg C]) to provide four stereoisomers (Rt: Peak 1:2.36 min, Peak 2:2.63, Peak 3:2.89, Peak 4:3.31).

Peak 1 (947650): 11.3 mg (98.5% purity, 100% ee, 0.5 equiv Diethylamine solvate). LCMS m/z=434.6 (M+H)+. 1H NMR (500 MHZ, METHANOL-d4) δ ppm 7.57-7.64 (m, 1H), 7.39 (t, J=1.83 Hz, 1H), 7.18-7.31 (m, 2H), 7.11 (d, J=7.33 Hz, 1H), 7.02 (s, 1H), 5.13-5.26 (m, 1H), 3.84 (s, 3H), 3.06-3.14 (m, 1H), 3.03 (q, J=7.33 Hz, 2H), 2.35 (dd, J=16.79, 4.58 Hz, 1H), 2.25-2.29 (m, 3H), 2.03-2.18 (m, 2H), 1.30 (t. J=7.33 Hz, 3H), 1.12-1.20 (m, 6H), 0.80-0.86 (m, 3H).

Peak 2 (947651): 14.0 mg (95.9% purity, 94.7% ee). LCMS m/z=434.6 (M+H)+. 1H NMR (500 MHz, METHANOL-d4) δ ppm 7.61 (s, 1H), 7.37-7.48 (m, 1H), 7.27-7.34 (m, 1H), 7.24 (t, J=7.63 Hz, 1H), 7.10-7.15 (m, 1H), 7.01 (s, 1H), 5.01-5.25 (m, 1H), 3.84 (s, 3H), 2.97-3.11 (m, 1H), 2.18-2.33 (m, 5H), 2.08-2.17 (m, 1H), 1.20 (s, 3H), 1.14 (d, J=1.22 Hz, 3H), 0.81 (t, J=7.33 Hz, 3H).

Peak 3 (947652): 15.2 mg (96.7% purity, 84.3% ee). LCMS m/z=434.6 (M+H)+. 1H NMR (500 MHZ, METHANOL-d4) δ ppm 7.61 (s, 1H), 7.41 (t, J=1.83 Hz, 1H), 7.28-7.34 (m, 1H), 7.24 (t. J=7.63 Hz, 1H), 7.12 (dt, J=7.63, 1.37 Hz, 1H), 7.01 (s, 1H), 5.08-5.23 (m, 1H), 3.84 (s, 3H), 3.01-3.10 (m, 1H), 2.19-2.33 (m, 5H), 2.07-2.16 (m, 1H), 1.20 (s, 3H), 1.14 (d, J=1.22 Hz, 3H), 0.81 (t, J=7.33 Hz, 3H).

Peak 4 (947653): 9.7 mg (98.4% purity, 83.8% ee). LCMS m/z=434.6 (M+H)+. 1H NMR (500 MHz, METHANOL-d4) δ ppm 7.62 (s, 1H), 7.37-7.42 (m, 1H), 7.24-7.29 (m, 1H), 7.20-7.24 (m, 1H), 7.09-7.13 (m, 1H), 7.02 (s, 1H), 5.08-5.26 (m, 1H), 3.84 (s, 3H), 3.05-3.13 (m, 1H), 2.31-2.39 (m, 1H), 2.28 (s, 3H), 2.06-2.18 (m, 2H), 1.10-1.19 (m, 6H), 0.83 (t, J=7.33 Hz, 3H).

### Preparation of 4-ethyl-8-fluoro-4-(3-(3-fluoro-5-methoxypyridin-4-yl)phenyl)-7,7-dimethyl-2,4,6,7,8,9-hexahydro-SH-pyrazolo[3,4-b]quinolin-5-one

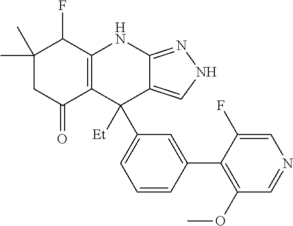 [SMILES: CCC1(c2cccc(-c3c(F)cncc3OC)c2)C2=C(Nc3n[nH]cc31)C(F)C(C)(C)CC2=O]

4-ethyl-8-fluoro-7,7-dimethyl-4-(3-(4,4,5,5-tetramethyl-1,3,2-dioxaborolan-2-yl)phenyl)-2,9-bis((2-(trimethylsilyl) ethoxy)methyl)-2,4,6,7,8,9-hexahydro-5H-pyrazolo[3,4-b]quinolin-5-one (231 mg, 318 μmol), 4-bromo-3-fluoro-5-methoxy-pyridine (65.5 mg, 318 μmol), [2-(2-aminophenyl)phenyl]-methylsulfonyloxy-palladium; dicyclohexyl-[2-(2,4,6-triisopropylphenyl)phenyl]phosphane (13.5 mg, 15.9 μmol), and potassium phosphate tribasic (135 mg, 636 μmol) were added to a reaction vial. The vial was then sealed and degassed by evacuating and backfilling with nitrogen. Dioxane (848 uL) and water (424 uL) were then added and the mixture was stirred at 100° C. for 24 hours. The mixture was then cooled to room temperature, concentrated, and used directly in the next step. LCMS m/z=725.8 (M+H)+.

4-ethyl-8-fluoro-4-[3-(3-fluoro-5-methoxy-4-pyridyl)phenyl]-7,7-dimethyl-2,9-bis(2-trimethylsilylethoxymethyl)-6,8-dihydropyrazolo[3,4-b]quinolin-5-one (230 mg, 317 μmol) was dissolved in TFA (1.50 mL) and CH₂Cl₂ (4.50 mL) and stirred at room temperature for 3 hours under nitrogen. The mixture was then concentrated and purified by column chromatography (5-100% [3:1 EtOAc:EtOH] in heptanes). The residue obtained was then purified by chiral SFC (CHIRALPAK IB 30×250 mm, 5 um, Method: 30% MeOH w/0.1% DEA in CO₂ [flow rate: 100 mL/min, ABPR 120bar, MBPR 40 psi, column temp 40 deg C]) to provide four stereoisomers (Rt: Peak 1:2.44 min, Peak 2:2.91, Peak 3:3.30, Peak 4:4.04).

Peak 1 (948545): 9.8 mg (95.7% purity, 100% ee). LCMS m/z=465.5 (M+H)+, 1H NMR (500 MHZ, METHANOL-d4) δ ppm 8.22 (s, 1H), 8.17 (s, 1H), 7.46 (d, J=8.55 Hz, 1H), 7.39 (s, 1H), 7.31 (t, J=7.94 Hz, 1H), 7.14 (d, J=6.71 Hz, 1H), 7.01 (s, 1H), 5.09-5.22 (m, 1H), 3.85 (s, 3H), 3.00-3.09 (m, 2H), 2.34 (dd, J=16.48, 4.88 Hz, 1H), 2.14 (d, J=16.48 Hz, 1H), 2.06-2.11 (m, 1H), 1.15 (d, J=8.55 Hz, 6H), 0.81 (t, J=7.33 Hz, 3H).

Peak 2 (948546): 13.1 mg (98% purity, 97.8% ee). LCMS m/z=465.6 (M+H)+. 1H NMR (500 MHz, METHANOL-d4) δ ppm 8.24 (s, 1H), 8.19 (s, 1H), 7.49 (d, J=7.94 Hz, 1H), 7.43 (d, J=1.83 Hz, 1H), 7.33 (t, J=7.63 Hz, 1H), 7.14-7.19 (m, 1H), 7.02 (s, 1H), 5.11-5.25 (m, 1H), 3.85-3.91 (m, 3H), 2.98-3.09 (m, 1H), 2.21-2.34 (m, 2H), 2.06-2.15 (m, 1H), 1.20 (s, 3H), 1.13 (d, J=1.22 Hz, 3H), 0.81 (t, J=7.33 Hz, 3H).

Peak 3 (948547): 13.3 mg (96.8% purity, 95.1% ee). LCMS m/z=465.6 (M+H)+1H NMR (500 MHz, METHANOL-d4) δ ppm 8.22 (s, 1H), 8.18 (s, 1H), 7.48 (d, J=9.16 Hz, 1H), 7.42 (s, 1H), 7.32 (t, J=7.63 Hz, 1H), 7.15 (d, J=6.71 Hz, 1H), 7.00 (s, 1H), 5.09-5.23 (m, 1H), 3.86 (s, 3H), 3.02 (dd, J=12.82, 7.33 Hz, 1H), 2.18-2.31 (m, 2H), 2.09 (dd, J=12.82, 6.71 Hz, 1H), 1.18 (s, 3H), 1.11 (d, J=1.22 Hz, 3H), 0.79 (t, J=7.33 Hz, 3H).

Peak 4 (948548): 8.7 mg (97% purity, 100% ee). LCMS m/z=465.6 (M+H)+. 1H NMR (500 MHZ, METHANOL-d4) δ ppm 8.21 (s, 1H), 8.17 (s, 1H), 7.46 (d, J=8.55 Hz, 1H), 7.39 (s, 1H), 7.31 (t, J=7.63 Hz, 1H), 7.14 (d, J=7.33 Hz, 1H), 7.01 (s, 1H), 5.08-5.25 (m, 1H), 3.85 (s, 3H), 3.04 (dd, J=12.51, 7.63 Hz, 1H), 2.34 (dd, J=16.79, 4.58 Hz, 1H), 2.14 (d, J=17.09 Hz, 1H), 2.09 (dd, J=12.82, 7.33 Hz, 1H), 1.15 (d, J=9.77 Hz, 6H), 0.81 (t, J=7.33 Hz, 3H).

### Preparation of 4-ethyl-8-fluoro-7,7-dimethyl-4-(3-(trifluoromethyl)phenyl)-2,4,6,7,8,9-hexahydro-5H-pyrazolo[3,4-b]quinolin-5-one

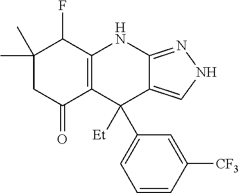 [SMILES: CCC1(c2cccc(C(F)(F)F)c2)C2=C(Nc3n[nH]cc31)C(F)C(C)(C)CC2=O]

Step 1: 1H-pyrazol-3-amine (164 mg, 1.98 mmol), 1-[3-(trifluoromethyl)phenyl]propan-1-one (400 mg, 1.98 mmol, 333 uL), and pTSA (37.7 mg, 198 μmol) were combined then toluene (4.00 mL) was added and the mixture was heated to 110° C. for 16 hours. The reaction was then cooled to room temperature and concentrated and the crude residue was purified by column chromatography (0 to 30% MeOH in CH₂Cl₂) to give 4-[1-[3-(trifluoromethyl)phenyl]prop-1-enyl]-1H-pyrazol-3-amine (510 mg, 1.91 mmol, 96.4% yield). LCMS: Rt=1.25 min, m/z 268.1.

Step 2:4-fluoro-5,5-dimethyl-cyclohexane-1,3-dione (79.9 mg, 505 μmol) and 4-[(E)-1-[3-(trifluoromethyl)phenyl]prop-1-enyl]-1H-pyrazol-3-amine (135 mg, 505 μmol) were combined and dissolved in TFA (1.00 mL) and the mixture was heated to 110° C. for 4 hours. The mixture was then cooled to room temperature and concentrated. The crude residue was purified with preporatory HPLC to give 4-ethyl-8-fluoro-7,7-dimethyl-4-[3-(trifluoromethyl)phenyl]-2,6,8,9-tetrahydropyrazolo[3,4-b]quinolin-5-one (37.0 mg, 90.8 μmol, 18% yield) as a mixture of isomers. LCMS: Rt=1.59 min, m/z 408.2.

The mixture of isomers was then purified by chiral SFC (CHIRALPAK IB 30×250 mm, 5 um, Method: 30% Methanol with 0.1% diethyl amine in CO₂ [flow rate: 100 mL/min, ABPR 120bar, MBPR 40 psi]) to provide four stereoisomers (Rt: Peak 1:2.22 min, Peak 2:2.63, Peak 3: 3.05, Peak 4:3.41).

Peak 1 (837646): 40.4 mg (95% purity, 100% ee). LCMS: Rt=1.76 min, m/z 408.2. 1H NMR (400 MHZ, CHLOROFORM-d) δ 7.46-7.71 (m, 2H), 7.30-7.44 (m, 2H), 6.85 (s, 1H), 4.16-4.49 (m, 1H), 3.11 (dd, J=7.53, 12.80 Hz, 1H), 2.36-2.61 (m, 2H), 1.84-2.18 (m, 1H), 1.20 (s, 3H), 1.17 (s, 3H), 0.82 (t, J=7.28 Hz, 3H).

Peak 2 (837683): 28.2 mg (95% purity, 100% ee). LCMS: Rt=1.73 min, m/z 408.2. 1H NMR (400 MHZ, CHLOROFORM-d) δ 7.60 (s, 2H), 7.36 (br. s., 2H), 6.90 (s, 1H), 4.26-4.56 (m, 1H), 2.93-3.16 (m, 1H), 2.41-2.59 (m, 2H), 1.95-2.14 (m, 1H), 1.23 (s, 3H), 1.08 (s, 3H), 0.77 (t, J=7.40 Hz, 3H).

Peak 3 (837753): 13.3 mg (95% purity, 100% ee). LCMS: Rt=1.73 min, m/z 408.2. 1H NMR (400 MHZ, CHLOROFORM-d) δ 7.60 (s, 2H), 7.36 (br. s., 2H), 6.90 (s, 1H), 4.31-4.61 (m, 1H), 2.95-3.14 (m, 1H), 2.35-2.59 (m, 2H), 1.90-2.19 (m, 1H), 1.23 (s, 3H), 1.08 (s, 3H), 0.77 (t, J=7.28 Hz, 3H).

Peak 4 (837803): 16.9 mg (95% purity, 100% ee). LCMS: Rt=1.75 min, m/z 408.2. 1H NMR (400 MHZ, CHLOROFORM-d) δ 7.60 (s, 2H), 7.36 (s, 2H), 6.86 (s, 1H), 4.18-4.52 (m, 1H), 2.98-3.26 (m, 1H), 2.34-2.60 (m, 2H), 1.93-2.14 (m, 1H), 1.20 (s, 3H), 1.17 (s, 3H), 0.82 (t, J=7.28 Hz, 3H).

### Example 5. Elucidation of GSK3α Structure Leads to Design Potent, Paralog Selective Small Molecule Inhibitors

Several GSK3α-selective inhibitors were developed and their activity evaluated across several in vitro and in vivo models of tau phosphorylation. The crystal structure for GSK3α both bound to a selective inhibitor and in apo form was obtained. Using this structure, compounds were designed with up to ˜20-fold selectivity for GSK3α over GSK3β and favorable drug-like properties. A rat model of tau phosphorylation and a chemoproteomic approach demonstrated that acute GSK3α inhibition can lower tau phosphorylation at disease-relevant sites in vivo with a high degree of selectivity over GSK3β and other kinases.

### Methods:

### Cell Culture and Transfection.

HEK293 or SH-SY5Y cells were obtained from American Type Culture Collection (ATCC, Manassas, VA) and cultured in Dulbecco's modified Eagle's medium (DMEM) supplemented with 10% fetal bovine serum (FBS), 10 units/ml penicillin, and 10 μg/ml streptomycin (all reagents were purchased from Gibco, Waltham, MA). Cell cultures were maintained in a humidified 5% (v/v) CO₂/air environment at 37° C. When the cells reached 50-80% confluence, they were transfected with a complex consisting of a 3:1 ratio of FuGENE 6 Transfection Reagent (Promega, Madison, WI) and plasmid DNA. Plasmid DNA was balanced out across reactions using an empty plasmid so that the same amount of DNA was added per well. This transfection complex was prepared following the manufacturers protocol and added directly to the cell media for 24 or 48 hours prior to lysis.

### SDS-PAGE and Western Blot.

Samples were lysed in Pierce RIPA lysis and extraction buffer (Thermo Fisher Scientific, Waltham, MA) supplemented with 1% Halt protease and phosphatase inhibitor cocktail (Thermo Fisher Scientific, Waltham, MA) and centrifuged at 14000×g for 20 min at 4° C. to clear the lysate. Protein concentrations were determined using the Direct Detect Infrared Spectrometer (EMD Millipore Corp., Burlington, MA) and 10 μg of each sample was denatured in 6×SDS sample buffer (Boston Bioproducts, Ashland, MA) for 7 minutes at 90° C. Proteins were loaded into a Criterion 7.5% tris-glycine gel (Bio-Rad, Hercules, CA) and separated by SDS-PAGE at 120V for 135 minutes. The gel was transferred to an IBlot2 nitrocellulose membrane (Invitrogen, Carlsbad, CA), blocked with TBST blocking buffer (Li-cor Biosciences, Lincoln, NE) for 1 hour, and washed three times with TBST. The membrane was probed with primary antibodies (1:1000 dilution) in antibody dilution buffer (1:1 TBST blocking buffer and 1×TBST) overnight at 4° C. The blot was then washed in triplicate with TBST and incubated for 1 hour with secondary antibody (1:10,000 dilution of IRDye 800 anti-mouse IgG and IRDye 680 anti-rabbit IgG, Li-cor Biosciences, Lincoln, NE) in antibody dilution buffer. After a final triplicate wash with TBST, the blot was visualized using the Odyssey CLx imaging system (Li-cor Biosciences, Lincoln, NE).

Phospho-Tau: plate-based assays for epitopes Thr231 and AT8.

293T cells stably expressing human 2N4R Tau were transfected with appropriate cDNA plasmids. 24 hours following transfection cells were treated with compound in a 10-point dose-response (from 1000× stocks of compound) and lysates harvested 2 hours later with cell extraction buffer (Life Technologies Cat #BN0001) or RIPA Buffer with Halt protease/phosphatase inhibitors added.

For pThr231 assay: Lysate was diluted 1:100 in assay buffer and Total Tau and pTau T231 was assessed by plate-based ELISA as per manufacturer instructions (MSD Cat #K15121D-1). Total fluorescence was measured using an Envison plate reader.

For AT8 assay: FRET antibodies (Custom designed Cisbio assay kit; S202/T205 Tb and total Tau D2 FRET antibodies) were added to lysate in a 384 well assay plate at 1:4 ratio (antibody/lysate). After antibody addition, the plate was left at room temperature for 60 minutes incubation time. Plate was read with appropriate wavelength settings using an Envision plate reader (ex320 and em620 and em665).

### NanoBRET.

HEK293T cells were transiently transfected with NanoLuc®-GSK3-alpha or NanoLuc®-GSK3-beta constructs using FuGene®-HD (Promega). Next day, the live cell NanoBRETTM target engagement assay was performed. Cells were treated with serially diluted compound in addition to NanoBRETTM In-cell Kinase Tracer-8 at EC₅₀ concentration (87 nM for GSK3-alpha and at 120 nM for GSK3-beta) for 2 hours at 37° C.+5% CO₂ incubator. Immediately prior to BRET measurements, NanoBRETTM Nano-Glo® Substrate and Extracellular NanoLuc® Inhibitor were added on cells and incubated for 2-3 minutes at room temperature. Donor emission at 450 nm and acceptor emission at 610 nm were measured using a EnVision plate reader. Raw BRET ratio values were calculated by dividing the acceptor emission value by the donor emission value for each sample. Raw BRET units were converted to milliBRET units (mBU) by multiplying each raw BRET value by 1000. mBU values for compound treatments were normalized to DMSO treatment. Ki values are calculated using the formula: Ki=(IC₅₀)/(1+([Tracer]/EC₅₀)). All experiments were performed as biological triplicates with three technical replications.

β-catenin translocation Assay:

SH-SY5Y cells were seeded into a 96-well CellCarrier Ultra (Perkin Elmer) plate at a density of 1×105 cells/ml in complete DMEM. Sixteen hours following plating, media was changed to DMEM with 2% serum, and compound was added with a D300e Digital Dispenser (Tecan) for an incubation time of six hours. After compound incubation, media was removed, and cells were fixed with 4% PFA for 10 minutes at room temperature. Each well was washed three times with PBS-T and then blocked with buffer (1×PBS with 5% Normal Goat Serum and 0.3% Triton X-100) for 30 minutes at room temperature. β-Catenin primary antibody (Cell Signaling #8480) was added at 1:500 dilution in blocking buffer and the cells were allowed to incubate in primary overnight at 4° C. The following day, primary antibody was removed, each well was washed three times with PBS-T, and secondary antibody (1:2000 anti-rabbit AlexaFluor Plus 488 and 1:10,000 Hoechst 33342) was added and allowed to incubate for 60 minutes at room temperature protected from light. After one-hour, secondary antibody was removed and wells were washed four times with PBS-T before imaging on the Opera Phenix. Sixteen fields per well were imaged with a 20× water lens.

Images were quantified in the Columbus Image Data Storage and Analysis software. Cells were identified using the Hoechst nuclear stain to identify individual nuclei. Intensity of the nuclear stain was also used to determine cell viability (Viable cells <30% CV of Hoechst Intensity)

### Animal Use.

All experiments were conducted in compliance with the rules set forth by the Biogen Institutional Animal Use and Care Committee in accordance with the guidelines established in the National Institutes of Health Guide for the Care and Use of Laboratory Animals. These protocols were approved by the Biogen IACUC committee.

### Rat Developmental Model of Tau Phosphorylation

Sprague Dawley rats were bred in-house and litters were injected with compound, I.P. at post-natal day 10. Rats were then euthanized at indicated times following drug administration. For tissue collection, animals were euthanized via decapitation, brains were quickly removed, and bisected sagitally. The right cortex, was isolated, placed into 2 mL microtubes along with a single 5 mm stainless steel bead and the left cortex were placed into 2 mL Lysing Matrix A tubes (MP Biomedicals, Santa Ana, CA), Both tubes were immediately snap frozen in liquid nitrogen.

### Determination of Free Drug Concentration in the Brain

Aqueous buffered homogenate was created from cortical tissue via bead beater, at a consistent homogenization factor, which was normalized by tissue weight. Total drug in brain was determined by measuring total exposure in brain in ng/ml (cortical tissue) via LC-MS/MS. This was then corrected to free concentration via an experimentally derived fu,br using rapid equilibrium dialysis with brain homogenate.

### In Vivo Target Engagement Assay:

Chemoproteomics competition was used to evaluate inhibitor selectivity and target engagement in vivo. Frozen cortexes were prepared for homogenization with 1.2 mL of Lysis Buffer (50 mM Tris-HCl, 0.8% NP-40 ((octylphenoxy poly(ethyleneoxy) ethanol)), 5% glycerol, 150 mM NaCl, 1.5 mM MgCl2, 25 mM NaF, 1 mM sodium vanadate, 1 mM DTT, pH 7.5 and supplemented with protease inhibitors). Tissues were homogenized in FastPrep-24 5G instrument at maximum oscillations for 20 seconds. The resulting brain homogenate was clarified by centrifugation for 1 h at 145 000 g, 4° C. The protein concentration of the supernatant was determined by BCA assay and each sample was diluted to final concentration of 5 mg/mL with lysis buffer. Kinome enrichment was performed similarly as described previously (Reinecke et.al. 2019) with minor modifications. In short, in a 96 deep-well filter plate, one milliliter of the brain lysate (5 mg/mL protein concentration) was incubated with 200 uL (50% slurry) of probe functionalized beads for 30 minutes on an end-over-end rotator at 4° C. Beads were thoroughly washed in two steps with lysis buffer containing 0.4% and 0.2% NP-40, respectively, and eluted with 100 μL of a 2×LDS sample buffer supplemented with 20 mM DTT. Eluates containing the enriched kinome were alkylated with 40 mM iodoacetamide for 30 min in the dark, then separated on 4-12% NuPAGE for approximately 1 cm and stained with colloidal Coomassie. Gel bands were first cut and diced into ˜1 mm cubes, and then de-stained with 50/50 Acetonitrile/50 mM NH4HCO3 solution, followed by tryptic digestion overnight at 37° C. The tryptic peptides were extracted into 40/60 Acetonitrile/0.1% formic acid solution and dried in speed-vac. The dry peptide mixture was reconstituted in 15 μL of 0.1% trifluoro acetic acid and analyzed on ID nanoLC-MS/MS platform using a standardized 140 min method. Peptides were separated on an Easy-Spray column (75 μm×50 cm, PepMAP C18, 1.9 μm) at 250 nL/min, analyzed on a QExactive HF mass spectrometer at data dependent acquisition (DDA) mode with MS1 at 60,000 and MS2 at 15,000 resolution, respectively. Raw data was first QC checked using in-house developed software and was subsequently searched against the Swissprot human database using Andromeda integrated in Maxquant (V 1.6.38) with mass tolerance of 20 ppm (MS1) and 4.5 ppm (MS2). Carbamidomethylation of cysteine residues was set as fixed modifications and (ST) phosphorylation, methionine oxidation, and (NQ) deamidation were set as variable modifications. LFQ values generated in the protein groups file were used to create the selectivity and target engagement graphs. Both selectivity and target engagement data at different doses and multiple time points are reported percentage inhibition compared to the vehicle groups.

Protein Expression, Purification and x-Ray Crystallography:

GSK3α residues 98-446 were cloned into a his-tag-MBP-TEV-vector and grown in T.ni cells. Six litters of cell pellets were lysed in 50 mM Hepes 7.4, 150 mM NaCl, 1 mM TCEP with protease inhibitors. Cells were lysed using a microfluidizer at 15,000 PSI and centrifuged at 18,000 rcf for 30 minutes. The resulting supernatant was run over a 3×5 ml MBPTrap HP column and washed with the lysis buffer. The protein was eluted in the lysis buffer containing 20 mM maltose. Eluted protein was treated with 200 ul of TEC (20,000 units) at 4° C. and dialyzed overnight into 50 mM Hepes 7.4, 150 mM NaCl, 1 mM TECP. The cleaved protein was put over an MBPTrap column and the eluent was collected. This was then put over a 16/60 Superdex200 column in 50 mM Hepes 7.4, 150 mM NaCl, 1 mM TCEP. Finally, the protein was then concentrated to 6 mg/ml for crystallography.

### Crystallization and Data Collection and Refinement.

Sitting drop plates were setup with GSK3α at 6 mg/ml complexed with 0.5 mM of the small molecule inhibitor, and 0.5 mM of synthetic Axin peptide. The complex crystallized in 0.1M Bis-TRIS pH 5.0, 0.2M CaCl2, 5% Glycerol and 30% PEG3350. The crystals were cryoprotected in 0.1M Bis-TRIS pH 5.0, 0.2M CaCl2, 25% Glycerol and 30% PEG3350. Data collection was performed at the LRL-CAT beamline at APS on a MAR-CCD detector at 0.97 Å. The data merged and scaled using Aimless. A structure of GSK3β was used as a search model to do molecular refinement using phaser in Phenix. Refinement was also done using phenix. The statistics for the quality of the data is as seen in Table 15.

TABLE 15

| Data | BRD0705 | | BRD0705 | | 948546 bound | | 948546 bound | |
|---|---|---|---|---|---|---|---|---|
| Collection/ | bound to | | bound to | | to GSK3alpha- | | to GSK3beta- | |
| refinement | GSK3alpha- | | GSK3beta-axin | | axin | | axin | |
| statistics | axin complex | | complex | | complex | | complex | |
| PDB Code | 7SXF | | 7SXJ | | 7SXG | | 7SXH | |
| Space Group | C2 | | P2₁2₁2₁ | | C 2 | | P2₁2₁2₁ | |
| Cell Dimensions | | | | | | | | |
| a, b, c (Å) | 119, 55, 66 | | 68, 73, 82 | | 120, 55, 66 | | 68, 72, 79 | |
| α, β, γ (°) | 90, 97, 90 | | 90, 90, 90 | | 90, 97, 90 | | 90, 90, 90 | |
| Resolution (Å) | 1.94 | | 50-1.85 | | 2.4 | | 2.1 | |
| R_{sym}^{a} | 0.03 | | 0.03 | | 0.06 | | 0.06 | |
| I/σ | 24.3 | | 29.13 | | 12.26 | | 14.65 | |
| Multiplicity | 3.5 | | 6.6 | | 3.4 | | 6.5 | |
| Total/unique | 109474/31641 | | 237493/36167 | | 58008/16932 | | 155896/23943 | |
| reflections | | | | | | | | |
| Completeness (%) | 97.7 | (91.06) | 99.9 | (99.9) | 96 | (98) | 99.3 | (95.0) |
| Rwork (Rfree) | 20/23 | | 17.9/20.2 | | 23/26 | | 21/25 | |
| CC_{1/2} | 0.99 | (0.97) | 0.99 | (0.72) | 0.99 | (0.54) | 0.99 | (0.6) |

| | | | | |
|---|---|---|---|---|
| R.m.s.d. bond | 0.006 | 0.01 | 0.003 | 0.003 |
| distance (Å) | | | | |
| R.m.s.d bond angle | 0.89 | 1.00 | 0.55 | 0.97 |
| (deg) | | | | |
| Avg. protein B-value | 61.6 | 47.83 | 72.99 | 57.8 |
| (Å²) | | | | |
| Avg. solvent B-value | 53.4 | 48.22 | 57.39 | 53.7 |
| (Å²) | | | | |
| Ramachandran Values | | | | |
| Preferred | 97.6 | 97 | 97 | 98.2 |
| Allowed | 2.3 | 3 | 3 | 1.8 |
| Disallowed | 0 | 0 | 0 | 0 |

*The value in parentheses is for the highest resolution bin (approximate interval, 0.1 A),
^{a}Rsym = f°| f§hkl · < f§hkl > | f° f§hkl
^{b}Rwork = f° f§hkl| |Fo · |Fc| | / f° f§hkl|Fo| for all data except 5% which is used for the R_{free} calculation

### Results:

### Acute and Selective Inhibition of GSK3α can Reduce Tau Phosphorylation at Disease Relevant Sites

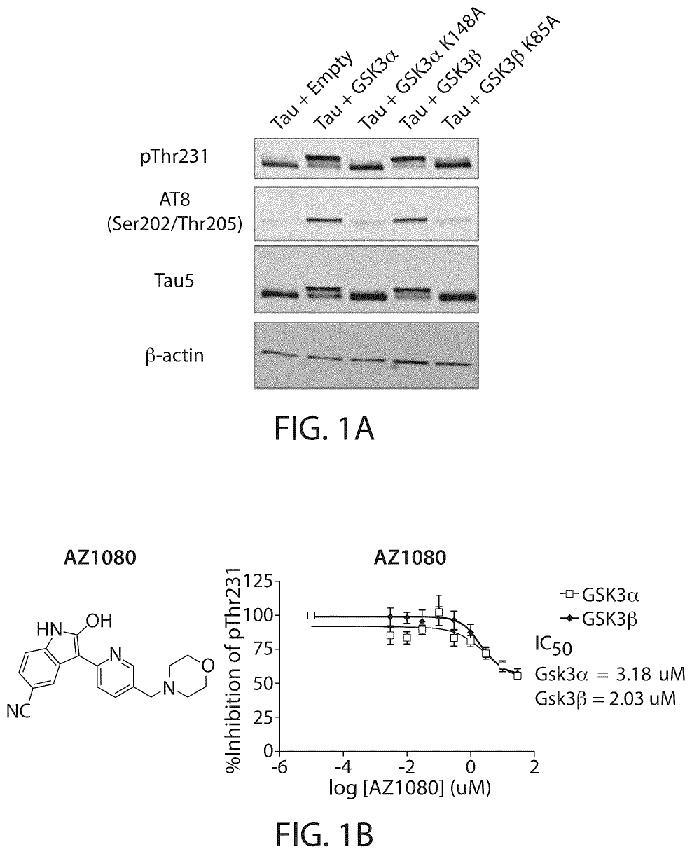

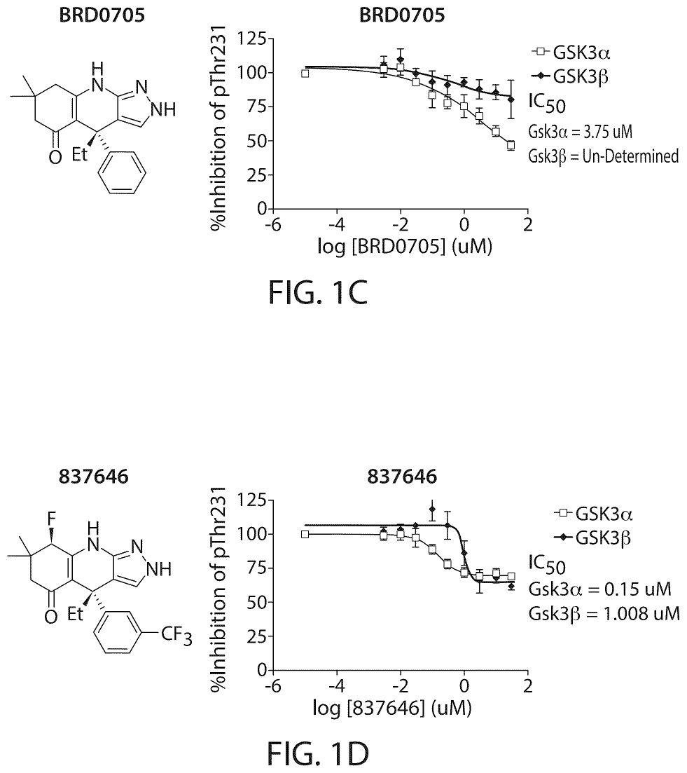

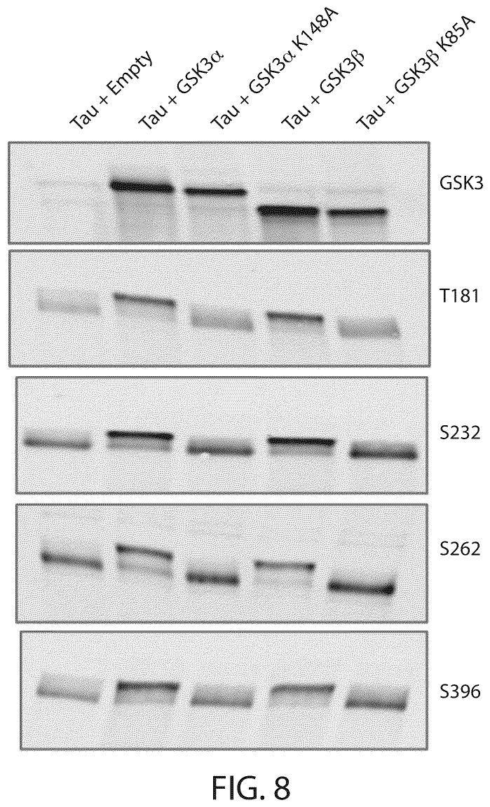

The hyper-phosphorylation of tau by GSK-3 has been implicated in Alzheimer's Disease (Llorens-Martin M et al . . . , 2014); however, the development of GSK paralog selective tool compounds is important for the field to investigate the relative contributions of GSK3α and/or GSK3β to different disease pathologies. To demonstrate the specific effects of each paralog on tau phosphorylation, HEK293 cells stably expressing human 2N4R tau (HEK-huTau) were transfected with plasmids expressing human GSK3α, human GSK3β, or kinase dead mutants in which the catalytic lysine was mutated to an alanine. Twenty-four hours after transfection, cells were lysed and run by SDS page to probe for phosphorylation of tau at different epitopes. The results demonstrate that both GSK3 isoforms phosphorylate tau at multiple epitopes including the disease enriched epitopes Thr231 and S202/Thr205 (AT8; FIG. 1A). Interestingly, when these kinases were expressed at approximately equal levels, selectivity of the GSK3 paralogs was not detected for specific phosphorylation epitopes on tau or a difference in total phosphorylation of tau (FIG. 1A, FIG. 8). This data indicates that in the current HEK-huTau cell assay, both GSK3α and GSK3β have comparable substrate affinity for several disease-enriched phospho-epitopes on tau.

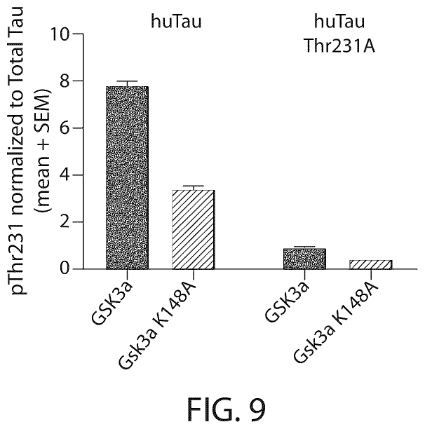

To test the potency and isoform selectivity of GSK3 small molecule inhibitors, HEK-huTau cells were transfected with plasmids containing either human GSK3α or GSK3β. Twenty-four hours after transfection, compounds were added at a 10-point dose response curve (at a range of 30 μM-3 nM) for a total of 2 hours, and cell lysates were probed for total tau and pThr231 using a plate-based assay. The results demonstrate that this assay is both sensitive to GSK3 activity and that the capture antibody is specific to pThr231 as mutating this Threonine residue to an Alanine abolished assay signal (FIG. 9). As a benchmark GSK3 inhibitor, the Astrazeneca compound AZ1080 (Georgievska et al . . . , 2013) was used. The results show similar potencies for this compound to both GSK3 isoforms (FIG. 1B; GSK3α IC₅₀=3.18 μM; GSK3β IC₅₀=2.03 μM). These results verify previous reports that this compound is not paralog selective. In addition, the reported GSK3α selective compound BRD0705 (Wagner et al . . . , 2018) was profiled. Selectivity of this compound was confirmed for GSK3α (FIG. 1C; GSK3α IC50=3.75 μM; GSK3β IC₅₀=Undetermined due to toxicity of this compound at doses >30 μM).

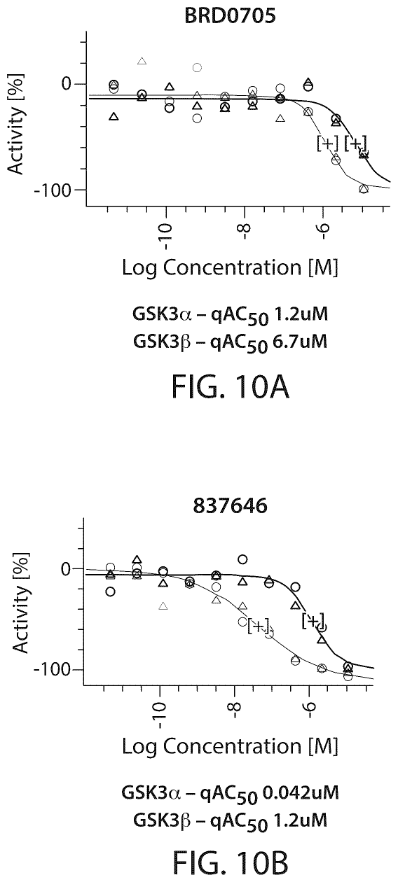

GSK3α selective compound 837646 was developed using structural analysis of the previously reported GSK3β D133E mutant, characterized, and underwent subsequent screening of functional group substitutions at both the phenyl and cyclohexyl ring. 837646 retains GSK3α paralog selectivity (FIG. 1C; GSK3α IC50=0.15 μM; GSK3β IC50=1 μM) while dramatically increasing cellular potency (GSK3α; BRD0705 IC50=3 μM, 837646 IC50=0.15 μM). Importantly, for BRD0705 and 837646, the relative cellular potency and paralog selectivity was similar when these compounds were tested for activity at an additional tau phosphorylation site pS202/pThr205 (AT8; FIG. 10) demonstrating the utility of these cell-based assays in future compound screening.

Reported GSK3 Small Molecule Inhibitors Bind at ATP Binding Pocket and have Similar Residence Times

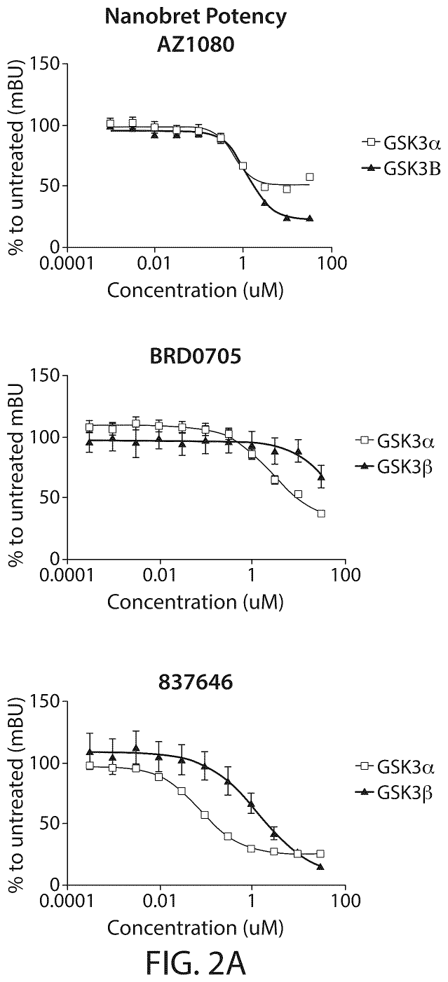

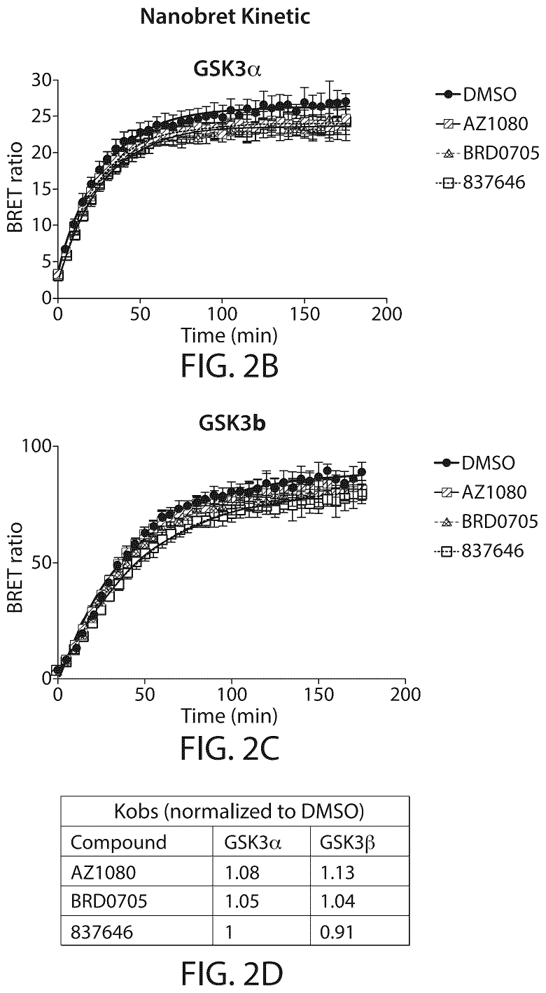

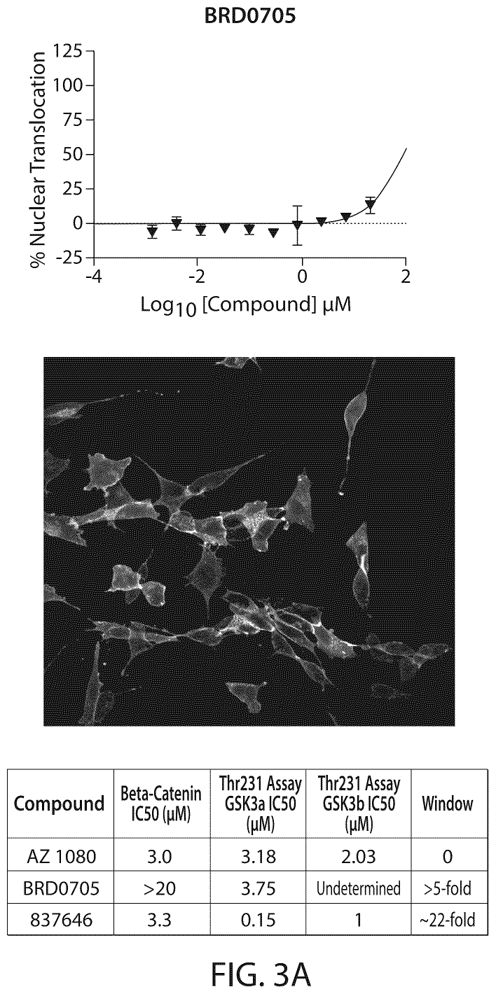

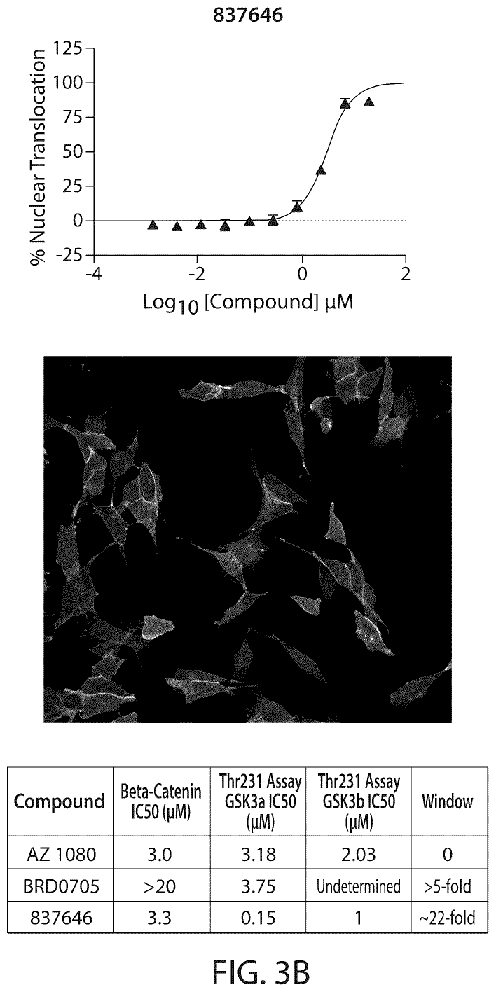

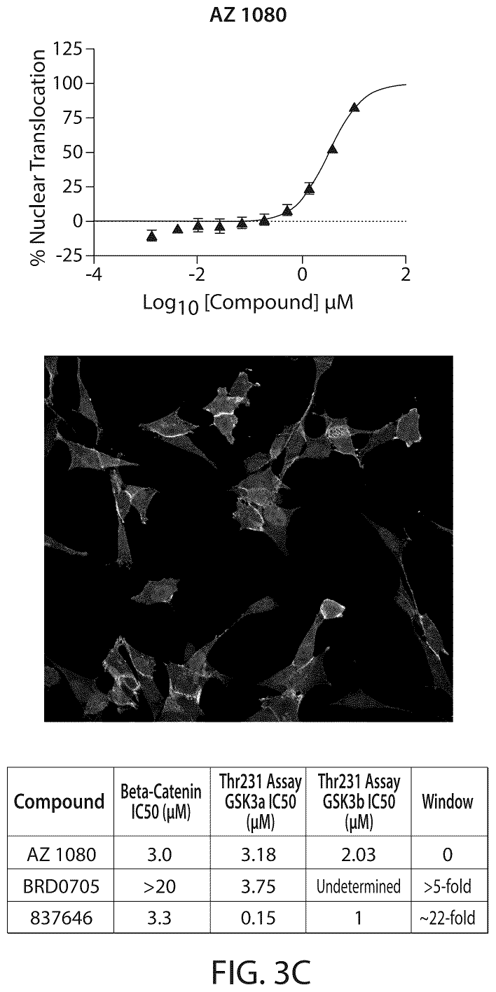

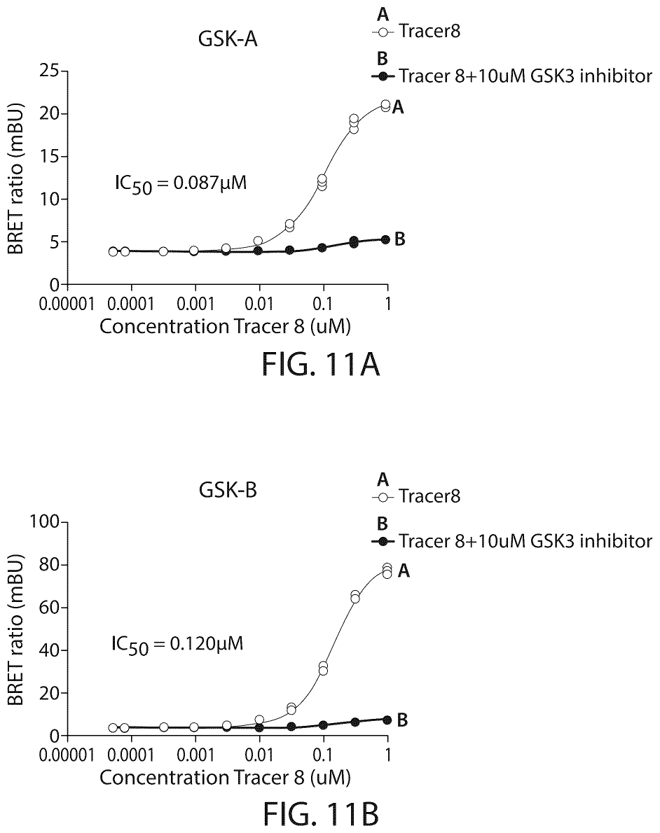

To further characterize the GSK3 inhibitor 837646, this compound was tested in a live-cell target engagement assay to investigate binding mode and residence time. To assess binding at the ATP pocket, HEK293T cells were transiently transfected with NanoLuc-GSK3α or NanoLuc-GSK3β plasmids and the following day treated for two hours with both inhibitors and the NanoBRET Target Engagement Kinase Tracer-8 (Promega; EC₅₀ concentration GSK3α=87 nM; GSK3β=120 nM; FIG. 11). In this assay, compound binding was quantified as loss of BRET signal from baseline due to competitive displacement of the tracer. The results show competitive displacement of the tracer with each of the inhibitors indicating that all compounds tested bind in the ATP pocket (FIG. 2A). Importantly, the rank order for compound potency and isoform selectivity was similar between the NanoBret assay and HEK293 tau phosphorylation assay providing additional confirmation of our previous results.

To investigate compound residence time, cells transfected with GSK3 NanoLuc plasmids were treated with inhibitors at 10× the previously identified IC₅₀ and BRET signal was measured every 5 min for a total of 3 hours after tracer addition. Overall, there was no difference in the residence time of compound 837646 at either GSK3α or GSK3β when compared to the previously profiled compounds AZ1080 or BRD0705. Together the data suggest the increase in potency observed with 837646 was not due to differences in binding mode or kinetics.

### Combined Inhibition of Both GSK3 Paralogs is Necessary for Nuclear Translocation of β-Catenin in SY5Y Cells

One concern in the clinical development of dual GSK3α/B inhibitors is the predicted stabilization and nuclear translocation of β-catenin (Yost et al . . . , 1996) whose mutation or overexpression is associated with many cancers (Iwao et al . . . , 1998; Kurnit et al., 2017).

Importantly, previous experiments have shown that selective genetic knockdown of either GSK3α or GSK3β does not increase β-catenin levels (Doble et al . . . , 2007). To determine whether 837646 shows a similar profile, β-catenin activation was measured by looking at nuclear translocation following compound addition. As expected, the non-selective compound AZ1080 lead to nuclear translation of β-catenin at a potency similar to what was previously reported for the tau phosphorylation assay (HEK-huTau assay GSK3α IC50=3.18 μM, GSK3β IC50=2.03 μM; SYSY β-catenin assay IC50=3 μM). The results show that compounds which had selectivity towards the GSK3α isoform (BRD0705 and 837646) had a significant window between their reported potency to effect tau phosphorylation by GSK3α and concentrations which affected β-catenin stabilization (BRD0705 >5-fold; 837646 >20-fold). The data demonstrate that inhibition of the GSK3α paralog alone was not sufficient to cause β-catenin activation because translocation to the nucleus was only observed with concentrations high enough to inhibit both paralogs (See Table 15). Together, these results demonstrate the identification of a novel GSK3α inhibitor with improved safety profile between inhibition of tau phosphorylation and β-catenin activation.

### Acute GSK3α Inhibition Reduces Tau Phosphorylation at Disease Relevant Sites In Vivo

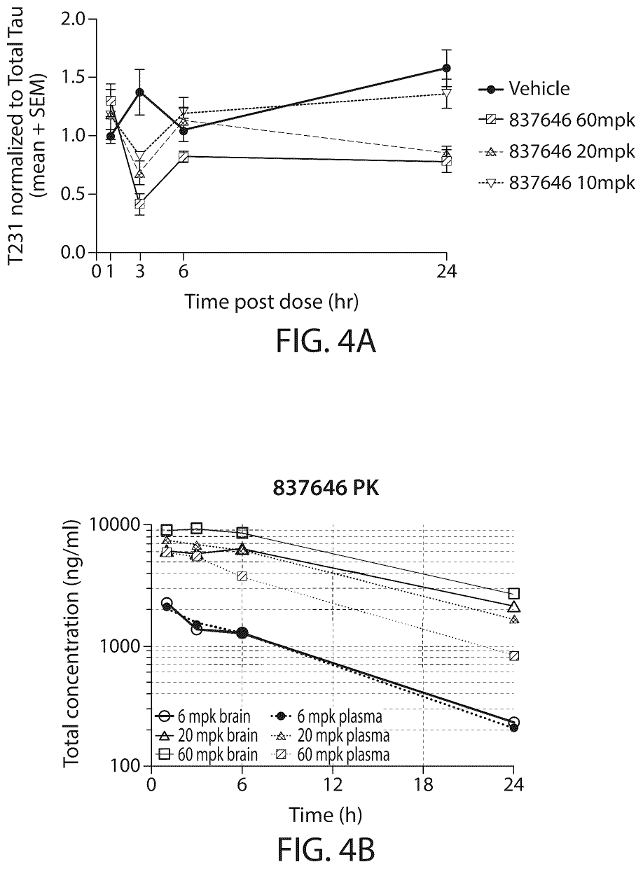

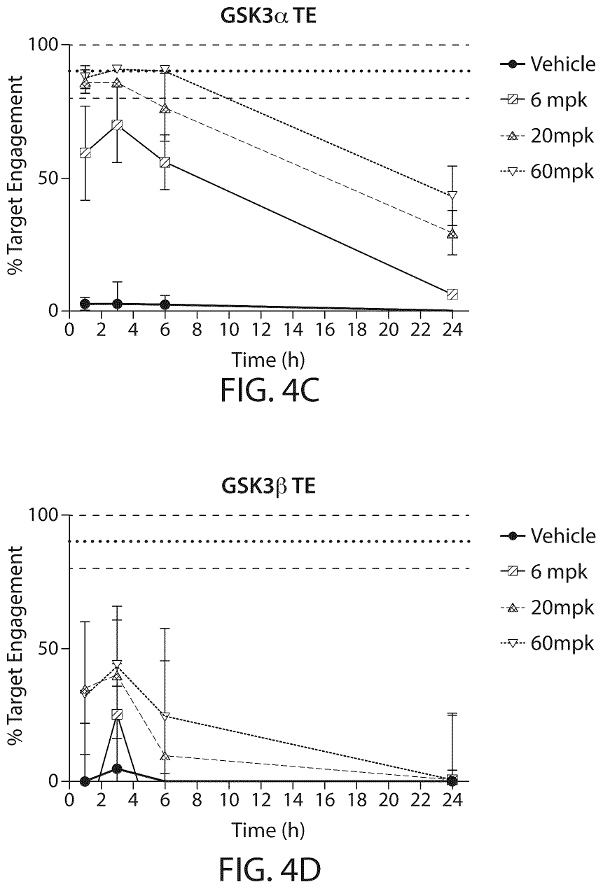

To determine whether a selective GSK3α inhibitor could also lower tau phosphorylation in a more physiological setting, 837646 was administered to rats at post-natal day 10. Previous experiments testing tau kinase inhibitors have demonstrated several advantages of using neonatal rats for in vivo PK/PD studies including the observation that these animals have a high level of basal tau phosphorylation (Halkina T et al . . . , 2021) and that compounds with less-than-ideal physical characteristics are able to cross the blood brain barrier due to the absence of Pgp transporters at this age (Matsuoka et al . . . , 1999). In the current experiment, 837646 was administered at 60, 20 and 10 mg/kg in P10 rats. The animals were sacrificed at several time points following treatment. The results demonstrate a significant lowering of tau phosphorylation at pThr231 beginning 3 hours after drug administration when compared to vehicle treated controls (FIG. 4A; 60 mpk p=0.004; 20 mpk p=0.0227). Interestingly, over 24 hours tau phosphorylation levels in the vehicle treated animals increased by 58% when compared to the previous baseline level (normalized tau phosphorylation at 1 hr and 24 hr in vehicle treated animals was 1 and 1.58 respectively). Animals injected with 837646 at either 20 or 60 mg/kg did not see a similar increase in tau phosphorylation over this time and were significantly different from the vehicle group at the 24 hr timepoint (60 mpk p=0.005; 20 mpk p=0.0139).

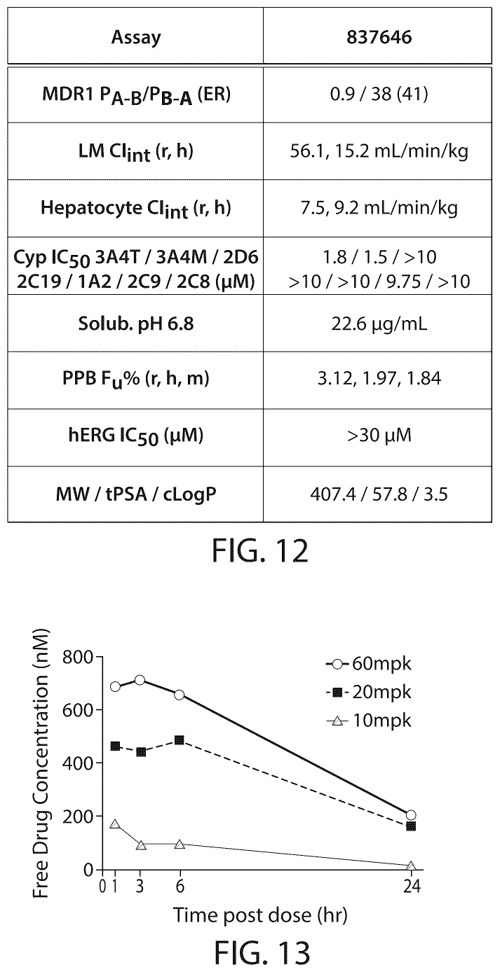

To determine drug exposure, blood and cortical tissue were analyzed at each time point following dosing. Overall, the pharmacokinetic data demonstrates dose responsive compound concentrations with a steady Cₘₐₓ extending 6 hours post injection (FIG. 4B). In addition, similar levels of the compound in both the blood and brain were observed (FIG. 4B), indicating that in neonatal rats, the compound had no issue crossing the blood brain barrier after peripheral injection. Free drug concentration in the brain was estimated based on plasma protein binding data (FIG. 12) and demonstrates a Cₘₐₓ of 715 nM for the 60 mpk dose, three hours after injection (FIG. 13). Together the data show a correlation between drug exposure and effects on tau phosphorylation levels.

To measure direct target engagement of the compound for both GSK3α and GSK3β competitive chemoproteomics methodology was used. Briefly, brain lysates were incubated with non-selective kinase inhibitors coupled sepharose beads (kinobeads) (Bantscheff et.al., 2007). Enriched proteins were analyzed by liquid chromatography coupled with tandem mass spectrometry. Target engagement was then measured as competitive displacement of 837646 with the non-selective beads. The results demonstrate that 837646 retained its selectivity for GSK3α over GSK3β in vivo (FIGS. 4D and 4E). In particular, three hours after dosing (a timepoint for which a significant decrease in tau phosphorylation at Thr231 was demonstrated), 837646 achieved over 90% target engagement for GSK3α (FIG. 4D) compared to 40% for GSK3β (FIG. 4E). At 24 hr, target engagement of 837646 at GSK3α was still close to 50% for both the 60 mg/kg and 20 mg/kg dose while there was no significant binding detected at this time point to the GSK3β paralog. The data suggest that the extended effect of this compound preventing tau phosphorylation at Thr231 was likely driven solely by GSK3α inhibition.

To investigate whether the effects of 837646 on tau phosphorylation were solely the result of GSK3 inhibition, additional kinases detected by mass spectrometry for compound engagement in the same brain lysates were profiled. The results show that at the highest dose tested (60 mg/kg) 7 out of 251 unique detected kinases (including GSK3α), which had target engagement greater than 70% (FIG. 4F and FIG. 13). Of the 6 off-target kinases detected none have previously been identified to share tau as a direct phosphorylation substrate. These results demonstrate that acute GSK3α inhibition can lead to significant reductions in tau phosphorylation in vivo.

### Elucidation of GSK3α Crystal Structure

The development of GSK3 paralog-selective inhibitors is complicated by the fact that, despite numerous reports over the past 20 years of structural information pertaining to GSK3β (Dajani R et al . . . , 2001), no crystal structures have been reported for GSK3α. Previously, Wagner et al. (2018) reported challenges associated with directly obtaining a crystal structure for GSK3 and instead utilized a GSK3β D133E mutant to mimic key differences between the two paralogs within the ATP binding site. From this structure it was speculated that a differential hydrogen-bonding outside of the ATP binding site may lead to alternative pockets allowing for paralog selective inhibitors. A previous report on the crystallization of GSK3β (Dajani et al . . . , 2003) described increased diffraction in the presence of its physiological binding partner Axin.

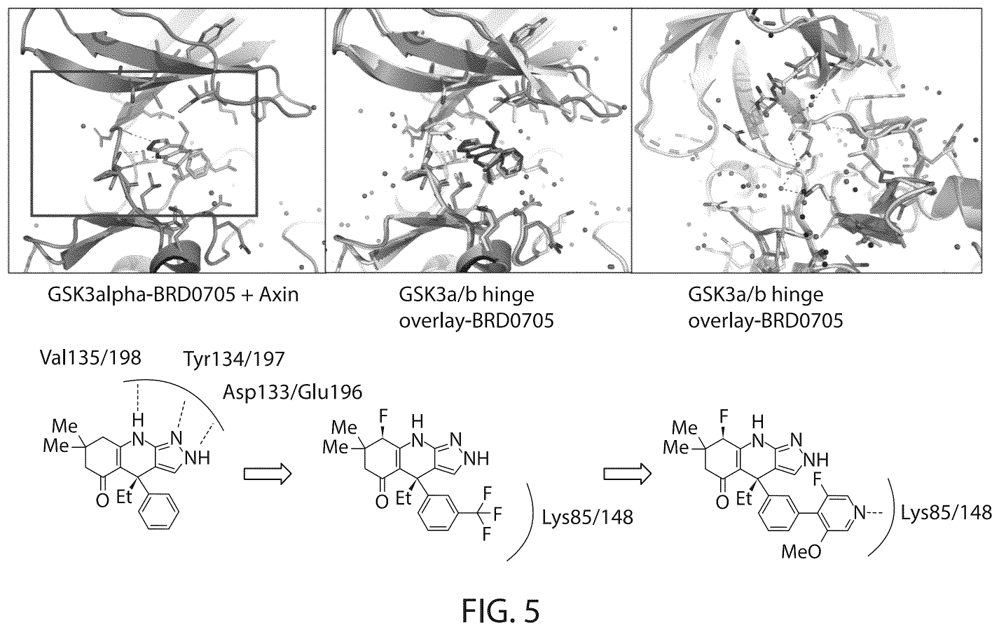

A first crystal structure of GSK3α, with an inhibitor (BRD0705) bound to the ATP active site was obtained (FIG. 5A). Crystallization of both GSK3α and GSK3β with Axin and BRD0705 led to differences in the hydrogen bonding network surrounding Glu196/Asp133 for each paralog (FIGS. 5A and SB). In particular, Glu196 had an interaction with Lys260 in GSK3«, whereas the corresponding interaction was not observed for GSK3β. Additionally, the two crystal structures contained subtle differences in the sidechain rotamers located within the ATP binding site surrounding BRD0705 which are represented in FIGS. 5B and 5C.

Since the primary difference in the two GSK3 active sites is situated at the hinge, substitutions adjacent to the aminopyrazole hinge-binding motif were considered. The crystal structure of BRD070S revealed that the binding pocket would likely tolerate substitution of the 3-position of the aryl ring. Indeed, as shown with 837646, fluorine substitution adjacent to the binge and an aryl trifluoromethyl substitution led to improved potency while maintaining GSK3α paralog selectivity. Next, using this crystal structure, additional compound derivatives were explored to further increase both the potency and selectivity of 837646. In particular, differential substitution of the phenyl ring was evaluated to provide improved paralog selectivity despite its position within the binding pocket. In some compounds, the trifluoromethyl group found in 837646 was replaced with heterocycle or pyridyl substituents, directing a hydrogen bond acceptor towards the catalytic lysine residue. This additional hydrogen bond interaction showed increased affinity of these compounds for either GSK3 paralog.

### Intelligent Design Leads to Novel, Potent GSK3α Inhibitors

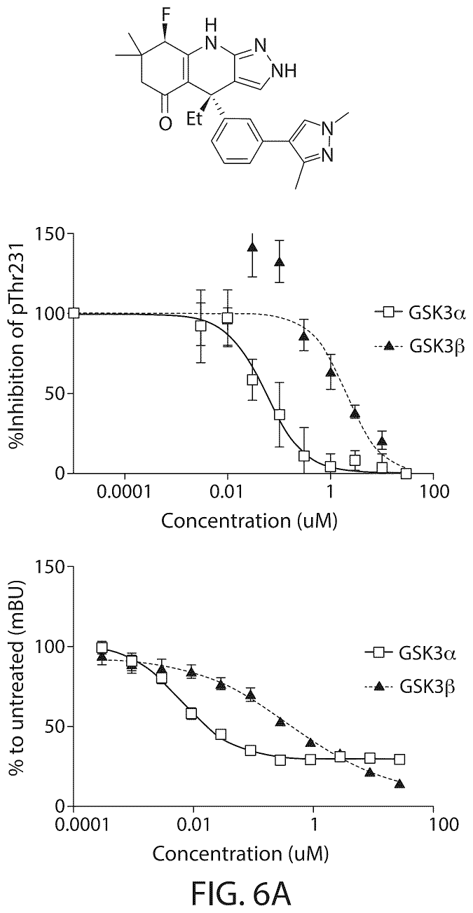

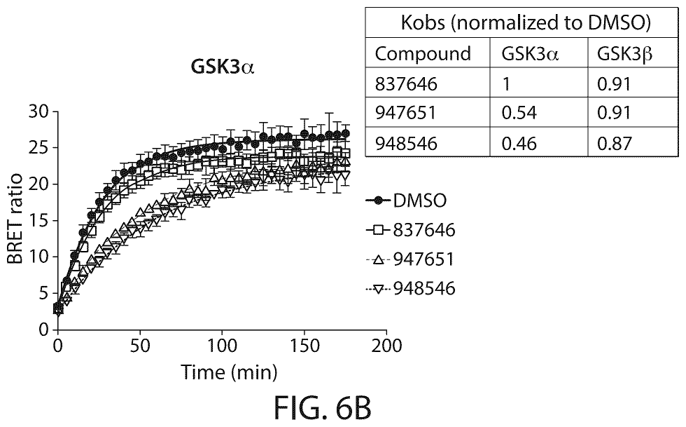

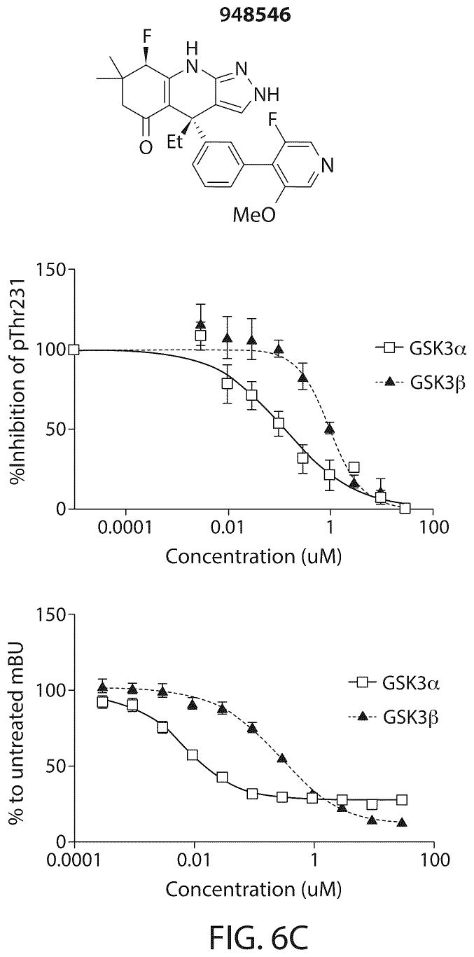

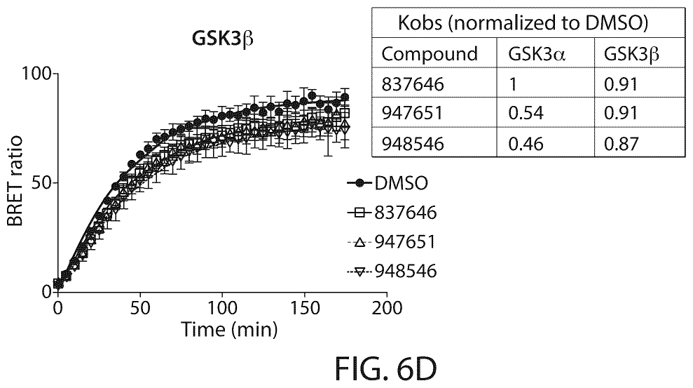

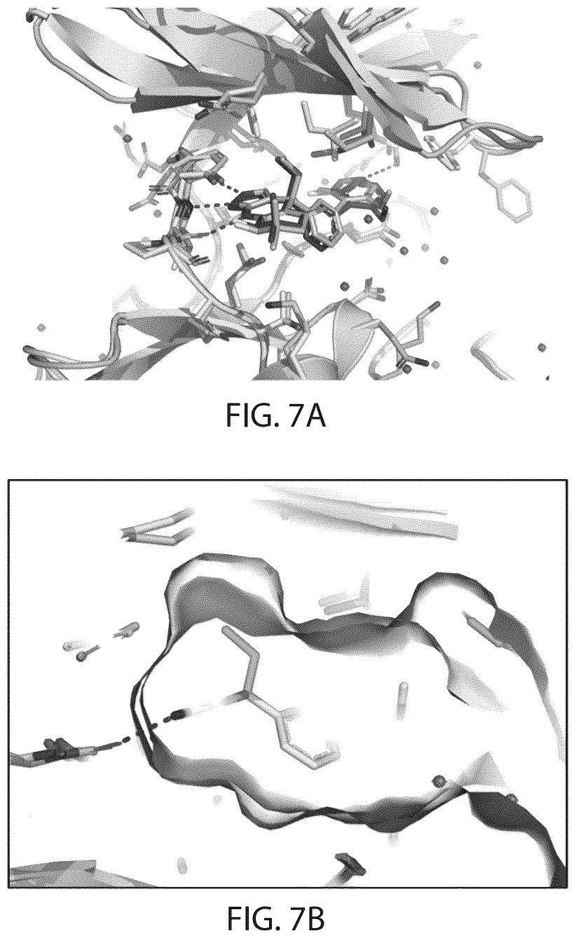

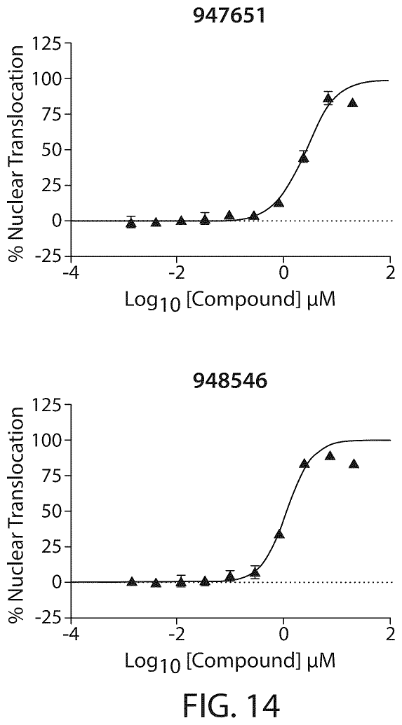

To determine whether the structure-based designs led to compound improvements, both 947651 and 948546 were tested in our buTau-HEK assay and their inhibition of tau phosphorylation at pThr231 was measured. Each of these compounds demonstrated a significant improvement in both potency and GSK3α selectivity when compared to the parent compound 837646 (FIG. 6A; 947651 GSK3α IC50=0.061 μM, GSK3β IC50=1.4 μM, 23-fold selective; 948546 GSK3α IC50=0.096 μM; GSK3β IC50=1 μM, 11-Fold selective). To measure more proximal target engagement, it was verified that 947651 and 948546 were ATP-competitive and direct GSK3 binders using the previously described nanobret tracer assay (FIG. 6B). Interestingly, both 947651 and 948546 show significantly longer residence times for GSK3α in comparison to 837646 (FIG. 6C). These results suggest that increasing ligand-protein interactions created a tighter interaction in the ATP pocket which subsequently led to slower dissociation. To verify that selective GSK3α inhibition with these compounds does not lead to β-catenin stabilization, the potency of these compounds to cause translocation of β-catenin to the nucleus in SYSY cells was measured. For each of these compounds, nuclear translocation of β-catenin occurred at concentrations in which both GSK3 paralogs are being inhibited (FIG. 14). These GSK3α selective compounds exhibited significant improvements in cellular potency compared to those previously reported in the literature.

Crystal Structure of GSK3α and GSK3β with the Novel 948546.

The in vitro characterization of 948546 demonstrates both a dramatic increase in compound potency and improvements in GSK30, paralog selectivity. To further validate that this was due to increased interactions of this compound with the GSK3α ATP pocket, crystallization of GSK3α was performed with Axin and 948546. The results show that 948546 interacts with Lys148/85 in both GSK3 paralogs which could account for some of the increased binding affinity. In addition, the crystal structure also demonstrates minor differences in the orientation of the biaryl motif via the lysine interaction between the GSK3α and GSK3β. The orientation of this biaryl motif relative to Lys148/85, and subsequent difference in hydrogen bonding enthalpy, may contribute to the observed paralog selectivity but more importantly highlights the binding differences of 948546 relative to BRD0705 and potential secondary effects on affinity. Interestingly, both the GSK3α and GSK3β structures with 948546 show that the central phenyl ring is rotated relative to BRD0705 and that this rotation may be needed for the biaryl ring to interact with Lys148/85. Additionally, the gem-dimethyl substituents on the solvent exposed ring adjacent to the hinge have undergone a pseudo chair-flip between the BRD0705 and 948546 structures and the axial methyl group is directed to the N-lobe rather than the C-lobe. Taken together, these differential conformational biases that are captured in the X-ray structures of 948546 may further contribute to selectivity by reinforcing the interaction of the ethyl moiety with the corresponding lipophilic pocket which was originally identified in BRD0705 Importantly, the structures of 948546 indicate that the ILE125(/62) residue in this lipophilic pocket is shifted relative to the 948546 GSK3β structure as well as both GSK3 structures of BRD0705 and provides an expanded binding site.

### Discussion:

Several GSK3α-selective inhibitors were developed and evaluated for their activity in both cellular and animal models of tau phosphorylation. The development of these compounds was aided by the first demonstration of a GSK3α crystal structure, and the results further describe how increasing ligand interactions with the GSK3α binding pocket led to significant improvements in compound potency and target residence time. Using a postnatal rat model and chemoproteomics, it was confirmed that acute, inhibition of GSK3α can suppress tau phosphorylation at epitopes enriched in disease. The selectivity of 948546 was demonstrated across the kinome. The current studies advance prior efforts to develop GSK3 paralog selective inhibitors and demonstrate therapeutically relevant activity both in vitro and in vivo.

The hyper-phosphorylation of tau has been shown to negatively affect a wide range of cellular processes including microtubule polymerization (Dillon et al . . . , 2020), axonal transport (Rodríguez-Martín T et al . . . , 2013), RNA translational selectivity (Koren et al . . . , 2019), nuclear import/export (Eftekharzadeh et al . . . , 2018), pre-synaptic vesicle motility (Zhou et al . . . , 2017) and post-synaptic excitotoxicity (Ittner et al . . . , 2010). However, the longest human tau isoform contains a total of 85 possible phosphorylation sites (Goedert et al . . . , 1989); making the identification of specific phosphorylation sites as drivers of disease difficult. GSK3 has been shown to phosphorylate tau at 42 different epitopes (Hanger et al . . . , 2011; Martin et al . . . , 2013) and the activity of GSK3 has been shown to correlate with the level of neurofibrillary tangles found in AD brains (Leroy et al . . . , 2002). It was demonstrated in a HEK293 cell system that both GSK3 paralogs phosphorylate tau at several epitopes (T181, S262, S396, Thr231, and AT8) with equal affinity and strength. To test whether acute GSK3α inhibition could also modulate tau phosphorylation in the rat brain a paralog selective inhibitor 837646 was tested at doses up to 60 mg/kg which at Cₘₐₓ engaged GSK3α over 90% while only occupying GSK3β at 40%. The results demonstrate a reduction in tau phosphorylation at Thr231 at all doses tested three hours after compound administration. Phosphorylation at Thr231 was examined because several lines of evidence indicate that phosphorylation at this epitope is an early event in in Alzheimer's Disease (Luna-Munoz et al . . . , 2005; Neddens et al . . . , 2018). In addition, phosphorylated tau at Thr231 is readily detectable in CSF and was found to be significantly higher in patients with dementia due to AD (Santos et al . . . , 2019). Importantly, this suggests the possibility that Thr231 phosphorylation could serve as a distal biomarker of GSK3 target engagement in the clinical development of similar compounds.

Chemoproteomics-based profiling allows for the simultaneous measuring of both target engagement and selectivity of kinase inhibitors (Reinecke et. al. 2019). In the current study, following administration of 837646, kinobeads were used to enrich for endogenous kinases and followed this with quantitative mass spectrometry to measure the differential enrichment of kinases over time at multiple doses. This approach showed a direct correlation between the free drug levels of our small molecule and target engagement at either the GSK3α or GSK3β active site. The data demonstrate that even at drug Cₘₐₓ (3 hr after injection of 60 mg/kg) the compound has selectivity for the GSK3α paralog. In addition, the results show that the kinobeads were able to label around 251 unique kinases in the rat brain at post-natal day 10. Of the 251 unique kinases identified, 837646 at Cₘₐₓ demonstrated greater than 70% target engagement at only 6 kinases apart from GSK30/GSK3β. Together these results demonstrate the selectivity of 837646 between GSK3 paralogs and across the kinome, while at the same time, establishing an expected range of target engagement in the tissue of interest.

947651 and 948546 showed significant improvements in cellular potency compared to previously described GSK3α inhibitors (Palomo V et al., 2012; Wagner F F et al . . . , 2018; Wang Y et al . . . , 2019) and, interestingly, significantly slower off rates when compared to their parent compound (Normalized Koff of Parent 837646=1 vs 7651=0.54; 8546=0.46). These data suggest that increased binding interactions within the GSK30 ATP pocket resulted in a tighter interaction with the target and therefore slower dissociation.

The development of GSK3α selective compounds demonstrates that selective inhibition of GSK3 paralogs can be obtained despite high binding site homology. Originally it was thought that selective inhibition would necessitate interaction with the one residue difference between GSK3α and GSK3β in the hinge region (Asp/Glu) due to all additional paralog specific side chains directed outside of the ATP pocket and therefore likely inaccessible to small molecules. Here it is shown that modification of the previously identified GSK3α inhibitor BRD0705 on the cyclohexyl ring and aryl substitution, led to 837646, which exhibited a significant improvement in cellular potency. This improvement may be due to substitutions adjacent and distal to the hinge binding region and was suggested in multiple assay systems as well as through determination of binding kinetics. This confirmed that despite highly homologous primary sequences for the active sites of the GSK3 paralogs, the projection and interactions of these residues occurs in a differential manner and leads to avenues to obtain selective binding with small molecule inhibitors. Further derivatization of the aryl functionality revealed that the biaryl moieties in 947651 and 948546 further improved in vitro potency and selectivity. Importantly, 948546 interacted with Lys148/85 after obtaining a crystal structure of this inhibitor with both GSK3 paralogs.

Previous attempts to use GSK3 inhibitors in the clinic have been plagued by various toxicities, driven in part by the simultaneous inhibition of both GSK3 paralogs resulting in β-catenin stabilization (Yost et al . . . , 1996). GSK3α selective inhibitors were investigated in the context of tau phosphorylation; however, GSK3 paralog-selective inhibitors have development potential across a multitude of disease indications. Differences in the crystal structures of GSK paralogs were used to drive potency and selectivity to the GSK3α. GSK3α has been implicated in the pathology of Fragile X syndrome and two separate studies have demonstrated that GSK3α, but not GSK3β, is overactive in the Fmr1-/y mouse hippocampus (Min et al . . . , 2019; McCamphill et al . . . , 2020). In series of recently published studies, McCamphill et al . . . , demonstrate that the GSK3α selective inhibitor BRD0705 corrected susceptibility to audiogenic seizures and reversed learning and memory deficits in a Fragile X mouse model (Fmr1-/y). Two compounds show significant improvements in potency when compared to BRD0705, which may increase the therapeutic window in different disease models.

### Example 6. GSK3 Inhibition of Compounds Useful in a Provided Method

TR-FRET Assay: The assay was performed in a white, 384 well, low volume, flat bottom plate (Grenier). After a 30 minute preincubation in assay buffer with the indicated concentration of compound, the kinase activity was initiated by the addition of the substrates. 1 nM GSK3α (GST-full length GSK3α [SignalChem G08-10G]) or 1 nM GSK3b (GST-full length GSK3b: [SignalChem G08-9G]) were then incubated at ambient temperature for 100 minutes in 10 μL of 50 mM Tris, pH 7.5, 20 mM MgCl2, 0.05 mM DTT, 100 μg/mL BSA and 1% DMSO, with 200 nM biotinylated peptide substrate (biotinylated and S645 phosphorylated glycogen synthase 631-650) and either 4 μM ATP (GSK3α, Table 16) or 2 μM ATP (GSK3b, Table 16) or 1 mM ATP (Table 17). The kinase activity was quenched with 2 μL of 250 mM EDTA. A 10 μL volume of 40 nM Strepavidin-d2, 2 nM Tb²⁺-pSer641 antibody in TR-FRET Detection Buffer (Invitrogen) was then added. After 60 min at room temperature the plate was read on Envision plate reader using Ex: 340 nm, Em: 615 nM and 665 nM. The normalized 665/615 Signal ratio versus log compound concentration was analyzed by XLFIT to yield IC₅₀ values.

TABLE 16

| | GSK3-A | GSK3-B | | GSK3-A | GSK3-B |
|---|---|---|---|---|---|
| | TR-FRET | TR-FRET | | TR-FRET | TR-FRET |
| | BIOCHEMICAL | BIOCHEMICAL | | BIOCHEMICAL | BIOCHEMICAL |
| | (TR-FRET); | (TR-FRET); | | (TR-FRET); | (TR-FRET); |
| Compound | GMean; IC₅₀ | GMean; IC₅₀ | Compound | GMean; IC₅₀ | GMean; IC₅₀ |
| number | (μM) | (μM) | number | (μM) | (μM) |
| 772965 | C | D | 947396 | A | A |
| 782424 | B | A | 947399 | B | C |
| 782425 | B | B | 947401 | A | B |
| 784440 | B | A | 947402 | A | B |
| 785735 | B | D | 947403 | A | A |
| 785736 | A | B | 947404 | A | A |
| 785737 | A | B | 947405 | A | A |
| 785738 | A | B | 947408 | A | A |
| 785739 | B | D | 947409 | C | D |
| 785762 | A | A | 947410 | A | B |
| 789009 | D | D | 947413 | C | D |
| 789010 | B | B | 947414 | A | A |
| 789011 | D | D | 947415 | C | D |
| 789013 | B | D | 947432 | A | B |
| 789277 | A | B | 947433 | D | D |
| 789312 | A | B | 947562 | A | A |
| 789330 | B | C | 947563 | A | B |
| 789331 | D | D | 947564 | B | B |
| 789332 | B | B | 947565 | A | A |
| 789333 | D | D | 947566 | C | C |
| 789334 | B | C | 947567 | A | A |
| 789335 | B | C | 947568 | A | A |
| 789336 | A | B | 947573 | B | C |
| 789337 | B | C | 947574 | A | A |
| 789457 | B | C | 947575 | A | A |
| 789458 | D | D | 947576 | A | C |
| 789459 | A | A | 947673 | A | B |
| 789460 | B | B | 947674 | A | B |
| 789461 | C | D | 947675 | A | B |
| 789462 | B | C | 947676 | A | B |
| 789497 | C | D | 947677 | A | A |
| 789504 | C | B | 947748 | A | B |
| 789505 | B | A | 947798 | A | B |
| 789513 | B | B | 947799 | A | A |
| 789514 | B | B | 947800 | A | A |
| 789524 | B | A | 947801 | A | A |
| 789560 | A | B | 947803 | A | A |
| 789561 | C | D | 947804 | A | B |
| 789562 | A | B | 947805 | B | C |
| 789563 | D | D | 947806 | A | A |
| 789564 | A | A | 947807 | A | B |
| 789565 | A | B | 947809 | B | C |
| 789566 | A | A | 947811 | A | A |
| 789583 | B | B | 947812 | A | A |
| 789584 | B | C | 947814 | A | A |
| 790611 | D | D | 947815 | C | D |
| 790612 | A | A | 947817 | A | A |
| 790613 | A | B | 947819 | A | A |
| 790614 | D | D | 947820 | B | B |
| 790615 | A | B | 947821 | A | A |
| 790616 | D | D | 947822 | B | D |
| 790617 | A | A | 947823 | B | C |
| 790618 | C | D | 947825 | B | D |
| 790619 | C | D | 947826 | A | A |
| 790632 | D | D | 947835 | A | B |
| 790730 | A | B | 947836 | A | B |
| 790733 | A | A | 947837 | A | A |
| 790734 | B | D | 947858 | A | A |
| 790745 | A | A | 947859 | A | A |
| 790754 | A | A | 947860 | A | A |
| 790755 | B | F | 947861 | A | A |
| 791256 | F | C | 947862 | A | A |
| 791257 | A | A | 947863 | A | A |
| 791262 | D | D | 947864 | A | B |
| 791263 | D | D | 947865 | A | A |
| 791264 | B | D | 947866 | A | A |
| 791265 | C | D | 947869 | A | A |
| 791271 | A | B | 947870 | A | B |
| 791272 | A | B | 947871 | A | B |
| 791273 | A | B | 947874 | B | C |
| 792336 | B | B | 947876 | A | A |
| 792337 | F | D | 947877 | A | A |
| 792338 | A | B | 947905 | A | B |
| 792339 | F | D | 947906 | A | B |
| 792340 | A | A | 947907 | C | D |
| 792341 | B | B | 947908 | C | D |
| 792342 | B | B | 947909 | B | C |
| 792343 | A | A | 947910 | A | A |
| 801308 | A | A | 947911 | A | A |
| 801309 | C | E | 947912 | B | C |
| 801310 | B | E | 948014 | B | D |
| 801311 | A | A | 948015 | A | A |
| 801312 | A | B | 948016 | D | D |
| 801313 | E | D | 948017 | A | A |
| 801314 | A | A | 948018 | A | A |
| 801315 | B | B | 948019 | C | D |
| 801316 | A | A | 948020 | B | B |
| 801317 | B | B | 948021 | B | D |
| 801318 | B | E | 948022 | A | A |
| 801319 | A | A | 948052 | A | B |
| 801320 | B | B | 948127 | B | D |
| 801321 | A | A | 948140 | A | B |
| 801322 | A | B | 948141 | C | D |
| 801323 | A | B | 948142 | A | A |
| 801324 | A | A | 948143 | A | B |
| 801351 | E | B | 948144 | A | B |
| 801352 | B | A | 948145 | B | D |
| 801356 | A | A | 948146 | C | D |
| 801357 | B | E | 948147 | A | A |
| 801358 | A | A | 948148 | C | D |
| 801366 | D | D | 948149 | A | B |
| 801367 | A | B | 948150 | A | A |
| 801368 | A | B | 948151 | B | D |
| 801369 | D | D | 948152 | A | B |
| 801397 | A | B | 948153 | D | D |
| 801412 | D | D | 948175 | A | A |
| 801413 | A | A | 948176 | A | A |
| 801445 | A | A | 948177 | A | B |
| 801446 | B | E | 948178 | A | A |
| 801447 | B | C | 948179 | A | A |
| 801448 | A | B | 948180 | A | A |
| 802358 | A | A | 948181 | B | B |
| 806568 | C | B | 948183 | C | D |
| 806569 | A | A | 948184 | A | B |
| 806603 | A | B | 948185 | A | A |
| 806604 | E | D | 948187 | A | A |
| 806605 | C | D | 948188 | C | D |
| 806607 | D | D | 948224 | A | B |
| 806608 | B | E | 948225 | C | D |
| 806609 | B | E | 948226 | A | B |
| 806611 | D | D | 948227 | C | D |
| 806632 | E | D | 948238 | A | B |
| 806633 | A | B | 948241 | A | B |
| 806637 | A | B | 948296 | B | C |
| 806938 | B | C | 948297 | A | A |
| 806939 | A | A | 948299 | A | C |
| 810358 | A | A | 948300 | A | B |
| 810359 | D | D | 948301 | A | A |
| 810360 | A | B | 948302 | A | B |
| 810362 | E | D | 948303 | A | A |
| 810363 | B | B | 948304 | A | A |
| 810364 | D | D | 948305 | A | A |
| 810365 | E | E | 948306 | A | B |
| 810366 | A | A | 948307 | A | A |
| 810367 | A | A | 948308 | D | D |
| 810368 | B | E | 948309 | C | D |
| 810369 | A | B | 948311 | B | C |
| 810371 | D | D | 948312 | A | B |
| 810373 | A | A | 948313 | A | A |
| 810375 | C | D | 948314 | A | A |
| 810376 | A | B | 948358 | A | A |
| 810377 | B | C | 948359 | A | A |
| 810379 | E | D | 948360 | C | D |
| 810450 | A | A | 948361 | A | A |
| 810451 | A | A | 948362 | D | D |
| 810452 | B | C | 948363 | C | D |
| 810453 | D | D | 948365 | C | D |
| 810460 | C | D | 948366 | C | D |
| 810464 | C | C | 948367 | B | D |
| 810466 | A | A | 948368 | A | B |
| 810484 | D | D | 948369 | D | D |
| 810485 | D | D | 948370 | A | B |
| 810486 | A | B | 948371 | A | A |
| 810487 | A | B | 948379 | A | B |
| 810488 | A | B | 948380 | B | D |
| 810489 | C | D | 948414 | D | D |
| 810495 | B | A | 948417 | B | C |
| 810506 | A | A | 948418 | A | A |
| 810507 | A | A | 948419 | A | A |
| 810508 | A | B | 948420 | A | B |
| 810509 | D | D | 948421 | A | A |
| 810510 | A | B | 948422 | B | B |
| 810590 | B | B | 948423 | A | B |
| 810591 | B | B | 948493 | D | D |
| 811555 | B | B | 948512 | A | A |
| 811556 | A | A | 948513 | A | B |
| 811557 | A | A | 948514 | C | D |
| 811558 | C | D | 948515 | A | A |
| 811559 | A | B | 948516 | A | B |
| 811561 | C | D | 948517 | A | A |
| 811563 | C | D | 948518 | D | D |
| 811564 | A | A | 948519 | D | D |
| 811565 | A | A | 948520 | B | C |
| 811567 | B | B | 948521 | A | C |
| 811595 | B | A | 948522 | B | D |
| 811596 | C | B | 948523 | A | B |
| 816752 | A | C | 948524 | C | D |
| 816795 | A | B | 948525 | A | A |
| 816796 | B | C | 948526 | C | D |
| 816797 | C | D | 948527 | A | A |
| 816798 | A | B | 948528 | A | B |
| 816799 | B | C | 948529 | A | B |
| 816800 | A | A | 948530 | B | C |
| 816837 | C | B | 948531 | B | D |
| 816838 | B | B | 948638 | D | D |
| 816839 | D | C | 948639 | A | B |
| 816840 | C | B | 948640 | A | A |
| 816871 | A | B | 948641 | D | D |
| 816872 | D | D | 948642 | B | C |
| 816873 | B | C | 948643 | D | D |
| 816874 | D | D | 948644 | A | A |
| 816875 | B | C | 948645 | B | D |
| 816876 | D | D | 948646 | C | D |
| 816877 | D | D | 948647 | D | D |
| 816878 | B | C | 948648 | D | D |
| 816879 | B | B | 948649 | D | D |
| 816880 | D | D | 948650 | D | D |
| 816881 | B | C | 948651 | D | D |
| 816882 | D | D | 948652 | B | D |
| 816884 | B | C | 948653 | A | A |
| 816885 | A | A | 948654 | D | D |
| 816886 | C | C | 948655 | A | B |
| 819560 | C | D | 948656 | D | D |
| 819561 | A | A | 948657 | D | D |
| 819564 | A | A | 948658 | D | D |
| 819565 | B | B | 948659 | D | D |
| 819568 | B | B | 948660 | A | A |
| 819569 | B | C | 948661 | A | B |
| 819570 | D | D | 948662 | C | D |
| 819571 | D | D | 948663 | D | D |
| 819572 | D | D | 948664 | C | D |
| 819573 | A | B | 948761 | A | A |
| 819574 | A | A | 948762 | A | A |
| 819575 | B | A | 948763 | A | B |
| 819576 | B | C | 948764 | A | B |
| 819577 | A | B | 948765 | A | A |
| 819622 | A | B | 948766 | A | B |
| 819638 | A | B | 948767 | A | A |
| 819639 | C | D | 948768 | A | A |
| 819640 | D | D | 948769 | B | B |
| 819641 | D | D | 948770 | B | B |
| 823720 | A | B | 948771 | A | A |
| 823721 | A | B | 948772 | B | B |
| 823724 | B | C | 948773 | C | D |
| 823726 | D | D | 948774 | A | A |
| 826651 | C | D | 948775 | A | A |
| 826652 | A | B | 948776 | B | B |
| 826653 | A | B | 948777 | A | A |
| 826654 | D | D | 948778 | A | A |
| 826655 | D | D | 948794 | A | B |
| 826715 | A | A | 948812 | A | A |
| 826716 | B | D | 948813 | C | D |
| 826717 | A | A | 948814 | D | D |
| 826718 | A | A | 948815 | A | B |
| 826719 | C | D | 948816 | D | D |
| 826764 | A | B | 948817 | D | D |
| 826766 | B | B | 948818 | D | D |
| 826767 | D | D | 948819 | C | D |
| 826768 | A | B | 948824 | C | D |
| 826769 | D | D | 948825 | B | C |
| 828845 | B | B | 948826 | C | D |
| 828847 | A | A | 948827 | A | B |
| 828848 | A | A | 948828 | A | B |
| 828849 | B | B | 948829 | D | D |
| 828850 | B | B | 948998 | A | A |
| 835310 | B | C | 948999 | A | B |
| 835312 | D | D | 949000 | B | B |
| 835313 | D | D | 949001 | B | B |
| 835314 | A | B | 949002 | C | D |
| 835315 | A | A | 949003 | B | B |
| 837646 | A | A | 949004 | A | B |
| 837683 | A | B | 949005 | A | A |
| 837753 | B | D | 949006 | A | B |
| 837803 | C | D | 949007 | C | D |
| 840857 | A | B | 949008 | C | D |
| 841868 | B | B | 949009 | D | D |
| 853910 | B | C | 949010 | A | A |
| 854926 | A | B | 949011 | A | B |
| 856947 | A | B | 949012 | A | B |
| 856948 | D | D | 949013 | A | A |
| 856949 | B | A | 949040 | A | B |
| 865168 | A | A | 949041 | C | D |
| 865169 | A | B | 949105 | A | B |
| 865170 | C | D | 949106 | C | D |
| 865171 | A | B | 949107 | D | D |
| 865172 | B | C | 949108 | A | A |
| 865194 | B | C | 949109 | A | B |
| 865195 | B | D | 949110 | C | D |
| 865196 | B | D | 949111 | B | C |
| 865198 | B | C | 949112 | A | B |
| 865199 | C | D | 949114 | C | D |
| 865201 | A | B | 949115 | A | A |
| 865202 | D | D | 949116 | D | D |
| 874260 | A | B | 949118 | A | B |
| 874261 | B | D | 949119 | C | D |
| 874294 | A | B | 949126 | A | B |
| 874298 | A | A | 949127 | C | D |
| 874299 | B | D | 949128 | A | B |
| 874312 | A | A | 949129 | D | D |
| 874314 | A | A | 949169 | A | A |
| 874315 | C | D | 949170 | A | A |
| 874316 | B | C | 949171 | A | A |
| 874317 | A | A | 949172 | A | A |
| 874318 | A | B | 949173 | A | A |
| 874323 | C | C | 949174 | A | A |
| 874324 | B | B | 949175 | A | A |
| 874510 | A | A | 949176 | C | D |
| 874511 | A | B | 949177 | A | A |
| 874531 | A | A | 949178 | B | C |
| 874533 | B | D | 949179 | B | D |
| 875568 | A | A | 949180 | A | B |
| 875608 | C | D | 949181 | A | A |
| 877310 | A | B | 949182 | A | A |
| 877311 | A | A | 949183 | A | A |
| 883850 | A | B | 949184 | C | D |
| 883851 | A | A | 949185 | A | A |
| 883852 | A | A | 949186 | B | B |
| 883866 | A | A | 949263 | D | D |
| 883872 | C | D | 949264 | D | D |
| 883873 | A | A | 949265 | C | D |
| 883874 | A | A | 949266 | A | B |
| 883875 | B | C | 949267 | B | C |
| 883984 | A | A | 949270 | A | B |
| 883985 | A | B | 949271 | B | D |
| 883986 | A | A | 949310 | A | B |
| 883987 | A | B | 949311 | B | D |
| 883988 | A | A | 949324 | A | A |
| 883989 | A | B | 949326 | A | B |
| 883990 | A | B | 949327 | C | D |
| 883991 | A | A | 949328 | C | D |
| 883992 | A | A | 949329 | C | D |
| 883993 | A | A | 949330 | A | B |
| 883994 | A | A | 949331 | A | B |
| 883995 | A | A | 949332 | C | D |
| 883996 | A | B | 949333 | A | B |
| 883997 | A | B | 949334 | C | D |
| 883998 | A | B | 949335 | D | D |
| 883999 | A | A | 949336 | A | A |
| 884000 | A | B | 949337 | B | C |
| 884001 | A | A | 949338 | A | A |
| 884002 | A | A | 949351 | A | B |
| 884003 | A | A | 949352 | A | A |
| 884109 | A | A | 949353 | A | B |
| 884110 | A | A | 949354 | B | C |
| 884111 | C | D | 949388 | C | D |
| 884112 | B | B | 949389 | C | D |
| 884115 | B | B | 949390 | C | D |
| 884116 | A | A | 949392 | C | D |
| 884117 | A | A | 949393 | C | D |
| 884118 | B | C | 949394 | A | B |
| 885088 | B | B | 949395 | C | D |
| 885099 | B | C | 949396 | D | D |
| 885110 | A | B | 949398 | A | B |
| 885120 | A | B | 949400 | C | D |
| 885188 | B | B | 949417 | A | A |
| 885204 | A | B | 949421 | B | D |
| 885217 | B | B | 949422 | A | A |
| 885228 | A | B | 949509 | D | D |
| 885237 | B | C | 949510 | D | D |
| 885331 | A | A | 949511 | D | D |
| 885344 | A | A | 949512 | C | D |
| 885355 | B | C | 949513 | D | D |
| 885367 | B | C | 949515 | D | D |
| 885382 | B | C | 949516 | C | D |
| 885402 | B | C | 949517 | C | D |
| 889556 | A | A | 949518 | B | D |
| 889557 | A | A | 949519 | D | D |
| 889558 | A | B | 949520 | A | B |
| 889565 | B | C | 949522 | A | B |
| 889572 | B | B | 949599 | B | B |
| 889579 | A | A | 949600 | D | D |
| 889580 | A | B | 949641 | D | D |
| 889581 | A | A | 949642 | B | C |
| 889582 | B | C | 949643 | D | D |
| 889604 | B | C | 949644 | B | C |
| 889622 | B | B | 949646 | D | D |
| 889631 | B | B | 949647 | B | D |
| 889632 | D | D | 949648 | A | A |
| 889639 | A | A | 949649 | A | A |
| 893305 | A | A | 949650 | A | A |
| 893306 | A | A | 949651 | A | A |
| 893309 | B | C | 949747 | A | A |
| 893316 | A | B | 949748 | A | B |
| 893323 | A | B | 949752 | D | D |
| 893324 | C | D | 949756 | D | D |
| 901477 | B | C | 949757 | A | A |
| 901478 | B | B | 949761 | A | B |
| 901479 | A | A | 949835 | A | B |
| 901480 | A | B | 949836 | C | D |
| 902202 | A | A | 949867 | A | B |
| 902969 | B | C | 949868 | B | D |
| 902970 | A | B | 949910 | A | A |
| 902971 | A | A | 949911 | B | D |
| 909201 | A | B | 949917 | A | A |
| 909202 | B | C | 949919 | A | A |
| 909203 | B | C | 950055 | A | A |
| 909204 | A | A | 950056 | B | D |
| 909205 | A | A | 950059 | A | A |
| 909212 | A | B | 950060 | A | B |
| 909213 | D | D | 950061 | B | D |
| 909214 | A | A | 950062 | A | B |
| 909215 | B | C | 950151 | A | C |
| 909216 | B | B | 950162 | B | B |
| 909217 | B | C | 950163 | A | A |
| 909218 | D | D | 950252 | A | A |
| 909219 | D | D | 950254 | A | C |
| 909220 | A | B | 950255 | B | D |
| 909223 | D | D | 950318 | A | A |
| 911969 | A | B | 950320 | A | A |
| 911970 | A | B | 950323 | A | A |
| 911972 | C | D | 950479 | A | B |
| 911975 | A | A | 950484 | A | A |
| 911976 | B | D | 950486 | B | C |
| 911980 | A | A | 950490 | B | B |
| 911981 | A | A | 950493 | B | B |
| 911988 | A | B | 950494 | A | A |
| 911989 | C | D | 950497 | A | A |
| 911990 | A | A | 950561 | A | B |
| 911991 | B | D | 950562 | A | B |
| 911992 | A | A | 950563 | A | A |
| 911993 | A | B | 950564 | A | B |
| 912003 | A | B | 950565 | A | B |
| 912004 | C | D | 950608 | B | C |
| 912064 | A | A | 950612 | A | C |
| 912065 | A | B | 950614 | A | B |
| 912066 | A | B | 950615 | A | B |
| 912067 | A | A | 950776 | A | B |
| 912068 | A | B | 950777 | A | B |
| 912069 | A | A | 950813 | A | B |
| 912070 | A | B | 950817 | D | D |
| 912071 | A | A | 950821 | B | C |
| 912078 | B | B | 950822 | B | B |
| 912079 | B | B | 950824 | A | A |
| 912088 | B | D | 950825 | D | D |
| 912089 | B | B | 950828 | A | B |
| 912090 | B | D | 950830 | A | B |
| 912091 | D | D | 951592 | D | D |
| 912105 | D | D | 951598 | B | D |
| 912166 | A | A | 951599 | B | B |
| 912167 | B | C | 951604 | A | B |
| 912169 | A | A | 951605 | C | D |
| 912171 | A | A | 951606 | A | A |
| 912172 | C | D | 951607 | A | A |
| 913015 | A | A | 951612 | A | A |
| 913016 | B | C | 951618 | B | C |
| 913017 | B | B | 951706 | A | A |
| 913018 | A | A | 951707 | B | C |
| 913019 | B | C | 951733 | A | B |
| 913020 | A | A | 951736 | A | B |
| 913021 | A | A | 951740 | A | A |
| 913022 | B | C | 951742 | B | C |
| 913023 | A | A | 951836 | B | C |
| 913024 | B | C | 951837 | B | B |
| 913025 | A | A | 951838 | B | C |
| 913026 | B | B | 951840 | A | B |
| 913027 | A | A | 951842 | B | C |
| 913028 | A | A | 951843 | B | D |
| 913029 | A | A | 951844 | A | B |
| 913030 | A | A | 951845 | B | B |
| 913031 | A | A | 951848 | A | B |
| 913032 | A | A | 951849 | A | B |
| 913033 | B | C | 951939 | A | B |
| 913034 | C | D | 951944 | C | D |
| 913035 | A | A | 951946 | B | C |
| 913036 | A | A | 951948 | A | A |
| 913037 | A | B | 951949 | A | B |
| 913038 | B | C | 951954 | D | D |
| 913039 | C | D | 952016 | B | C |
| 913040 | B | C | 952017 | A | B |
| 913041 | C | D | 952018 | B | C |
| 913042 | D | D | 952019 | A | B |
| 913043 | A | A | 952021 | A | A |
| 913044 | B | B | 952022 | B | C |
| 913045 | A | A | 952023 | B | C |
| 913046 | C | D | 952024 | B | D |
| 913047 | A | A | 952025 | A | A |
| 913048 | B | C | 952207 | B | C |
| 913049 | B | D | 952208 | B | D |
| 913050 | A | A | 952209 | A | A |
| 913051 | B | D | 952210 | A | B |
| 913052 | A | A | 952257 | A | B |
| 913053 | A | A | 952262 | D | D |
| 913054 | B | C | 952410 | A | A |
| 913121 | A | B | 952411 | C | D |
| 913122 | B | B | 952478 | C | D |
| 913123 | A | A | 952479 | D | D |
| 913124 | B | B | 952560 | A | A |
| 913125 | A | B | 952561 | B | B |
| 913134 | B | C | 952563 | C | D |
| 913135 | A | A | 952565 | A | B |
| 913136 | A | B | 952566 | B | C |
| 913137 | D | D | 952629 | A | B |
| 913140 | A | B | 952630 | A | B |
| 913141 | B | C | 952636 | D | D |
| 913219 | A | B | 952638 | D | D |
| 913222 | A | B | 952754 | A | B |
| 913223 | D | D | 952758 | A | B |
| 913252 | B | B | 952760 | D | D |
| 913253 | A | A | 952909 | A | B |
| 913254 | A | A | 952910 | A | B |
| 913255 | A | B | 952912 | B | D |
| 913256 | C | D | 952913 | A | B |
| 913257 | A | A | 957497 | D | D |
| 913258 | A | A | 957518 | A | B |
| 913274 | A | A | 861716 | C | D |
| 913310 | A | A | 948205 | A | A |
| 913311 | A | A | 949742 | B | B |
| 913334 | A | B | 949743 | A | A |
| 913346 | A | B | 949082 | A | A |
| 913347 | A | A | 949397 | A | A |
| 913348 | A | B | 949399 | C | D |
| 913349 | A | A | 949514 | C | D |
| 913350 | B | A | 949521 | A | A |
| 913351 | A | A | 949639 | B | B |
| 913352 | A | A | 949805 | B | C |
| 915316 | A | A | 949840 | B | C |
| 915340 | B | C | 950272 | A | B |
| 915400 | A | A | 950273 | D | D |
| 915401 | B | C | 950282 | A | B |
| 915630 | B | C | 950283 | C | D |
| 917076 | A | A | 950815 | B | C |
| 917079 | B | C | 950818 | B | C |
| 917086 | A | A | 950819 | B | C |
| 917087 | B | C | 950826 | D | D |
| 917088 | A | A | 950827 | D | D |
| 917089 | B | C | 951565 | A | B |
| 917130 | A | B | 951566 | C | D |
| 917131 | A | A | 951702 | A | B |
| 917132 | A | A | 951710 | D | D |
| 917146 | A | A | 952205 | C | D |
| 917147 | A | B | 952212 | A | B |
| 917148 | C | D | 952557 | A | B |
| 917149 | B | C | 952562 | C | D |
| 917150 | A | A | 952564 | A | A |
| 917151 | C | D | 952752 | A | B |
| 917152 | A | B | 952757 | C | D |
| 917209 | A | B | 952759 | A | A |
| 917210 | D | D | 826714 | D | D |
| 917212 | A | B | 841866 | D | D |
| 917226 | B | B | 841867 | D | D |
| 917289 | B | C | 877288 | D | D |
| 917291 | A | A | 877294 | D | D |
| 917292 | B | B | 880765 | D | D |
| 917293 | A | A | 884020 | D | D |
| 918271 | A | A | 889570 | D | D |
| 918272 | B | B | 889571 | D | D |
| 918273 | A | A | 893313 | D | D |
| 918274 | A | B | 893315 | D | D |
| 918279 | A | A | 901472 | D | D |
| 918280 | A | A | 901473 | D | D |
| 918281 | A | B | 911958 | D | D |
| 918321 | A | A | 912077 | D | D |
| 918322 | D | D | 912107 | D | D |
| 918326 | B | B | 913265 | D | D |
| 918327 | C | D | 924310 | D | D |
| 919840 | A | A | 929436 | D | D |
| 924312 | B | C | 929438 | D | D |
| 924504 | B | B | 929439 | D | D |
| 924520 | D | D | 929441 | D | D |
| 924543 | C | D | 929448 | D | D |
| 926304 | B | C | 932633 | D | D |
| 926836 | A | B | 932650 | D | D |
| 926840 | A | A | 932651 | D | D |
| 926844 | A | B | 932652 | D | D |
| 927338 | A | B | 932654 | D | D |
| 927339 | C | D | 943544 | D | D |
| 927340 | D | D | 943545 | D | D |
| 927341 | C | C | 943914 | D | D |
| 927342 | A | A | 810378 | E | E |
| 927344 | A | A | 909206 | A | A |
| 927345 | D | D | 913127 | A | A |
| 927346 | A | A | 913128 | A | A |
| 927347 | A | A | 913129 | A | A |
| 927348 | A | A | 889573 | A | B |
| 929387 | A | A | 912075 | C | D |
| 929398 | A | A | 912094 | C | D |
| 929399 | A | A | 912113 | C | C |
| 929400 | A | A | 909209 | C | C |
| 929401 | A | A | 912170 | C | D |
| 929402 | A | B | 946078 | C | D |
| 929442 | A | B | 932653 | C | D |
| 929443 | D | D | 949323 | C | D |
| 929444 | A | A | 884013 | C | D |
| 929445 | C | D | 889577 | C | C |
| 929447 | A | A | 884023 | C | D |
| 929449 | C | C | 889583 | C | D |
| 929450 | A | B | 893314 | C | D |
| 929451 | D | D | 819563 | C | D |
| 929452 | C | D | 946080 | C | C |
| 929453 | C | D | 810505 | C | D |
| 929454 | A | A | 874319 | C | C |
| 929455 | D | D | 819562 | C | D |
| 930334 | A | B | 874313 | C | C |
| 930565 | C | D | 929446 | C | C |
| 930566 | A | B | 947813 | C | C |
| 930569 | B | C | 874320 | C | C |
| 932625 | A | A | 924309 | C | D |
| 932626 | B | B | 917145 | C | C |
| 932627 | A | A | 884014 | C | D |
| 932628 | A | B | 924298 | C | C |
| 932629 | A | B | 884106 | C | C |
| 932630 | A | A | 945477 | C | C |
| 932631 | A | A | 884024 | C | D |
| 932634 | A | B | 786065 | C | E |
| 932635 | D | D | 889575 | B | C |
| 932636 | A | A | 912106 | B | C |
| 932637 | D | D | 826763 | B | C |
| 932638 | D | D | 907328 | B | C |
| 932642 | A | B | 889584 | B | C |
| 932643 | B | C | 907329 | B | C |
| 932644 | D | D | 945395 | B | C |
| 932645 | B | C | 913184 | B | C |
| 932646 | D | D | 874536 | B | C |
| 932647 | A | B | 884019 | B | C |
| 932655 | A | A | 899559 | B | C |
| 932656 | D | D | 924311 | B | C |
| 932657 | A | C | 877314 | B | C |
| 932658 | A | B | 826649 | B | C |
| 932662 | D | D | 884113 | B | C |
| 932663 | A | A | 826765 | B | C |
| 932664 | A | B | 883846 | B | C |
| 932665 | D | D | 786061 | B | E |
| 932666 | A | B | 913214 | B | C |
| 932667 | D | D | 889568 | B | C |
| 942642 | A | A | 949640 | B | C |
| 943492 | A | A | 883833 | B | C |
| 943514 | A | A | 913185 | B | C |
| 943515 | C | D | 945394 | B | C |
| 943516 | A | A | 874537 | B | B |
| 943517 | B | D | 943895 | B | B |
| 943518 | A | A | 899560 | B | C |
| 943519 | B | C | 912076 | B | C |
| 943531 | A | B | 874529 | B | C |
| 943532 | A | B | 883974 | B | C |
| 943533 | A | A | 889574 | B | B |
| 943534 | A | A | 913186 | B | C |
| 943535 | A | A | 883834 | B | C |
| 943536 | A | C | 893319 | B | B |
| 943537 | A | B | 913215 | B | C |
| 943538 | A | B | 901475 | B | B |
| 943539 | A | A | 913213 | B | C |
| 943540 | A | B | 883836 | B | C |
| 943541 | A | A | 947257 | B | C |
| 943542 | A | A | 901476 | B | B |
| 943546 | C | D | 917160 | B | B |
| 943547 | C | D | 952434 | B | B |
| 943913 | A | A | 945483 | B | B |
| 943915 | D | D | 912073 | B | B |
| 943918 | B | C | 884120 | B | B |
| 943919 | A | B | 912173 | B | B |
| 943988 | B | C | 883978 | B | B |
| 943989 | D | D | 945396 | B | B |
| 943990 | A | A | 949322 | B | C |
| 943991 | B | C | 883835 | B | B |
| 943992 | A | B | 884114 | B | B |
| 943993 | D | D | 945658 | B | C |
| 943994 | A | A | 889569 | B | B |
| 943995 | B | D | 913442 | B | B |
| 943996 | A | A | 913263 | B | B |
| 944017 | A | A | 912112 | B | B |
| 944018 | A | A | 901458 | B | B |
| 944019 | A | A | 949320 | B | C |
| 944023 | A | B | 826713 | B | B |
| 944024 | A | A | 884022 | B | B |
| 944025 | A | B | 947251 | B | B |
| 944026 | A | A | 947262 | B | B |
| 944029 | A | B | 913216 | B | B |
| 944031 | A | A | 945478 | B | B |
| 944032 | A | A | 909210 | B | B |
| 944033 | A | A | 896013 | B | B |
| 944034 | D | D | 918315 | B | B |
| 944035 | C | D | 945679 | B | C |
| 944036 | C | D | 949318 | B | B |
| 945328 | A | A | 924299 | B | B |
| 945329 | B | C | 950477 | B | B |
| 945458 | A | A | 889576 | B | B |
| 945463 | B | C | 877313 | B | B |
| 945466 | B | C | 826648 | B | B |
| 945467 | A | B | 874509 | B | B |
| 945468 | A | B | 884021 | B | B |
| 945469 | A | A | 918270 | B | B |
| 945471 | C | C | 883840 | B | B |
| 945472 | A | B | 945397 | B | B |
| 945474 | A | B | 912072 | B | B |
| 945475 | A | B | 924307 | B | B |
| 945476 | A | B | 924308 | B | B |
| 945479 | B | C | 883962 | B | B |
| 945481 | A | A | 883841 | B | B |
| 945482 | C | D | 952437 | B | B |
| 945484 | D | D | 893318 | B | B |
| 945485 | D | D | 945906 | B | B |
| 945486 | C | D | 947473 | B | B |
| 945527 | A | A | 913441 | B | B |
| 945528 | A | A | 893307 | A | B |
| 945529 | A | A | 946072 | A | B |
| 945530 | A | A | 909208 | A | B |
| 945531 | A | A | 918316 | A | B |
| 945532 | A | A | 909211 | A | B |
| 945543 | A | A | 946073 | A | B |
| 945544 | B | D | 874527 | A | B |
| 945545 | C | D | 924715 | A | B |
| 945546 | A | A | 912074 | A | B |
| 945547 | C | D | 897751 | A | B |
| 945548 | A | A | 932659 | A | B |
| 945549 | A | A | 889567 | A | B |
| 945550 | B | D | 932660 | A | B |
| 945551 | A | A | 918277 | A | B |
| 945552 | B | D | 917136 | A | B |
| 945600 | B | C | 945674 | A | B |
| 945601 | B | B | 952435 | A | B |
| 945605 | A | A | 889664 | A | B |
| 945606 | A | B | 874528 | A | B |
| 945607 | A | B | 926832 | A | B |
| 945609 | A | B | 951839 | A | B |
| 945610 | A | B | 917142 | A | B |
| 945612 | C | D | 945473 | A | B |
| 945613 | A | A | 917140 | A | B |
| 945615 | D | D | 883865 | A | B |
| 945616 | D | D | 947466 | A | B |
| 945618 | C | D | 947471 | A | B |
| 945619 | C | D | 913126 | A | B |
| 945620 | B | C | 945662 | A | B |
| 945703 | A | A | 927343 | A | B |
| 945704 | A | A | 932641 | A | B |
| 945708 | A | B | 893308 | A | B |
| 945710 | A | A | 917134 | A | B |
| 945713 | A | A | 918284 | A | A |
| 945715 | A | A | 883973 | A | B |
| 945724 | A | A | 874532 | A | A |
| 945725 | A | A | 947245 | A | B |
| 945726 | C | D | 786072 | A | A |
| 945728 | C | D | 948122 | A | B |
| 945729 | B | C | 883972 | A | B |
| 945730 | A | B | 884107 | A | B |
| 945933 | A | A | 932675 | A | B |
| 945939 | B | D | 790666 | A | A |
| 945943 | A | B | 945678 | A | B |
| 945958 | C | D | 884119 | A | B |
| 945992 | A | B | 932661 | A | B |
| 945995 | A | A | 951705 | A | A |
| 945997 | A | A | 883864 | A | B |
| 945998 | A | A | 951846 | A | B |
| 946000 | D | D | 874975 | A | A |
| 946002 | D | D | 942344 | A | B |
| 946003 | B | D | 948416 | A | B |
| 946055 | B | C | 897750 | A | A |
| 946056 | B | D | 893320 | A | A |
| 946089 | A | A | 897752 | A | B |
| 946090 | B | D | 932639 | A | B |
| 946091 | A | A | 917135 | A | B |
| 946092 | C | D | 917137 | A | A |
| 946093 | A | A | 932632 | A | B |
| 946094 | B | C | 947472 | A | B |
| 946095 | A | A | 874535 | A | A |
| 946096 | B | D | 926827 | A | A |
| 946097 | A | B | 947459 | A | B |
| 946098 | D | D | 947609 | A | B |
| 946099 | A | A | 951711 | A | A |
| 946100 | B | C | 912084 | A | A |
| 946101 | C | D | 917141 | A | B |
| 946102 | C | D | 943894 | A | B |
| 946103 | B | C | 893321 | A | A |
| 946104 | A | B | 947463 | A | B |
| 947324 | A | A | 947898 | A | B |
| 947325 | A | B | 918275 | A | A |
| 947326 | A | A | 945669 | A | A |
| 947327 | A | B | 948415 | A | A |
| 947328 | B | B | 826650 | A | A |
| 947329 | A | A | 947460 | A | A |
| 947333 | A | A | 949321 | A | A |
| 947334 | B | C | 877320 | A | A |
| 947336 | A | A | 918285 | A | B |
| 947337 | B | C | 943962 | A | B |
| 947363 | A | A | 947474 | A | A |
| 947364 | B | B | 948298 | A | A |
| 947365 | B | C | 951708 | A | B |
| 947372 | A | A | 884005 | A | A |
| 947383 | C | D | 917081 | A | A |
| 947384 | A | A | 947470 | A | A |
| 947397 | A | A | 951709 | A | B |
| 947398 | A | A | 942462 | A | A |
| 947400 | A | A | 889578 | A | A |
| 947406 | A | B | 918282 | A | A |
| 947407 | A | C | 947256 | A | A |
| 947411 | D | D | 947261 | A | A |
| 947412 | C | D | 951790 | A | A |
| 947434 | A | B | 917133 | A | A |
| 947490 | B | C | 943961 | A | A |
| 947491 | A | A | 945667 | A | B |
| 947492 | A | C | 947467 | A | B |
| 947493 | A | B | 947468 | A | A |
| 947494 | A | B | 951862 | A | A |
| 947498 | A | A | 912092 | A | A |
| 947499 | C | D | 913260 | A | A |
| 947511 | A | A | 913356 | A | A |
| 947512 | A | B | 947241 | A | A |
| 947548 | B | B | 947249 | A | A |
| 947569 | A | A | 950478 | A | A |
| 947570 | B | C | 917161 | A | A |
| 947571 | B | C | 942432 | A | A |
| 947650 | A | A | 945719 | A | A |
| 947651 | A | A | 947259 | A | A |
| 947652 | A | B | 948117 | A | A |
| 947653 | A | B | 918283 | A | A |
| 947655 | A | B | 945677 | A | B |
| 947656 | B | D | 947477 | A | A |
| 947657 | B | D | 951713 | A | A |
| 947658 | A | A | 952436 | A | A |
| 947659 | A | B | 917082 | A | A |
| 947664 | A | B | 926815 | A | A |
| 947665 | C | D | 932649 | A | A |
| 947802 | A | A | 943483 | A | A |
| 947808 | A | A | 945664 | A | A |
| 947810 | A | B | 947475 | A | A |
| 947816 | C | D | 947607 | A | A |
| 947818 | B | D | 947610 | A | A |
| 947824 | B | C | 947615 | A | A |
| 947867 | A | A | 917138 | A | A |
| 947868 | A | A | 932640 | A | A |
| 947872 | B | C | 947606 | A | A |
| 947873 | B | B | 947608 | A | A |
| 948057 | B | C | 951788 | A | A |
| 948058 | A | A | 951864 | A | A |
| 948060 | A | A | 884004 | A | A |
| 948061 | B | C | 913264 | A | A |
| 948088 | B | C | 932648 | A | A |
| 948089 | A | B | 945657 | A | B |
| 948090 | A | B | 945671 | A | A |
| 948091 | A | B | 945676 | A | A |
| 948092 | A | A | 947247 | A | A |
| 948093 | A | B | 947612 | A | A |
| 948545 | A | A | 947904 | A | A |
| 948546 | A | A | 951726 | A | A |
| 948547 | A | A | 918278 | A | A |
| 948548 | A | B | 943543 | A | A |
| 948615 | A | A | 947479 | A | A |
| 948779 | C | D | 947900 | A | A |
| 948793 | A | A | 949319 | A | A |
| 949083 | A | B | 865193 | A | A |
| 949103 | A | A | 880776 | A | A |
| 949104 | A | A | 945673 | A | A |
| 949113 | C | D | 945680 | A | A |
| 949131 | B | C | 947250 | A | A |
| 949132 | D | D | 947465 | A | A |
| 949268 | C | D | 947480 | A | A |
| 949391 | C | D | 947605 | A | A |
| 949401 | A | A | 947897 | A | A |
| 949753 | C | D | 947901 | A | A |
| 949754 | D | D | 901466 | A | A |
| 949759 | D | D | 901471 | A | A |
| 949760 | B | D | 918276 | A | A |
| 950160 | C | D | 943482 | A | A |
| 950161 | C | D | 943486 | A | A |
| 950164 | D | D | 943488 | A | A |
| 950165 | D | D | 943489 | A | A |
| 950324 | A | B | 945655 | A | A |
| 950325 | D | D | 945659 | A | A |
| 950326 | A | B | 945660 | A | A |
| 950329 | A | A | 945663 | A | A |
| 950330 | B | D | 945665 | A | A |
| 950331 | B | D | 945714 | A | A |
| 950332 | D | D | 947903 | A | A |
| 950333 | A | B | 948119 | A | A |
| 957196 | D | D | 950814 | A | A |
| 957197 | B | C | 951703 | A | A |
| 957199 | D | D | 951724 | A | A |
| 957200 | B | B | 874530 | A | A |
| 957403 | B | C | 901467 | A | A |
| 957404 | D | D | 913251 | A | A |
| 957405 | B | C | 917143 | A | A |
| 957406 | D | D | 942366 | A | A |
| 957504 | B | C | 943481 | A | A |
| 957505 | A | C | 945661 | A | A |
| 957506 | A | B | 945666 | A | A |
| 957507 | A | A | 947244 | A | A |
| 957508 | D | D | 947462 | A | A |
| 957509 | B | D | 947478 | A | A |
| 957510 | C | D | 947482 | A | A |
| 957511 | A | B | 947899 | A | A |
| 959341 | A | B | 948120 | A | A |
| 959342 | C | D | 948121 | A | A |
| 959343 | A | A | 901468 | A | A |
| 959344 | D | D | 901474 | A | A |
| | | | 929440 | A | A |
| | | | 943484 | A | A |
| 963155 | A | A | 947243 | A | A |
| | | | 947246 | A | A |
| | | | 947248 | A | A |
| 791266 | D | D | 947252 | A | A |
| 791268 | D | D | 947258 | A | A |
| 791269 | B | B | 947263 | A | A |
| 791270 | B | F | 947481 | A | A |
| 901464 | B | B | 947483 | A | A |
| 912080 | B | B | 947613 | A | A |
| 912081 | B | C | 947616 | A | A |
| 943916 | A | B | 889665 | A | A |
| 943917 | A | B | 893311 | A | A |
| 943920 | C | D | 901469 | A | A |
| 943921 | C | D | 913261 | A | A |
| 913355 | D | D | 917139 | A | A |
| 911957 | A | B | 943485 | A | A |
| 912104 | A | B | 945656 | A | A |
| 913156 | B | D | 945670 | A | A |
| 913198 | A | B | 945672 | A | A |
| 913451 | A | B | 945905 | A | A |
| 913452 | D | D | 947242 | A | A |
| 913454 | A | B | 947253 | A | A |
| 913473 | A | B | 947254 | A | A |
| 913475 | C | D | 947469 | A | A |
| 913495 | D | D | 947611 | A | A |
| 917157 | D | D | 947902 | A | A |
| 917158 | B | C | 948118 | A | A |
| 917159 | A | B | 948123 | A | A |
| 917162 | A | B | 951725 | A | A |
| 917294 | B | C | 893310 | A | A |
| 917295 | B | C | 901465 | A | A |
| 918251 | D | D | 901470 | A | A |
| 918252 | A | B | 913259 | A | A |
| 918313 | B | C | 913262 | A | A |
| 929388 | A | B | 917144 | A | A |
| 929389 | C | D | 929437 | A | A |
| 930330 | A | B | 945668 | A | A |
| 930331 | A | B | 945712 | A | A |
| 930332 | C | D | 947464 | A | A |
| 930333 | D | D | 947484 | A | A |
| 945367 | B | C | 947614 | A | A |
| 945368 | D | D | 951701 | A | A |
| 945376 | D | D | 951787 | A | A |
| 945377 | B | C | 951789 | A | A |
| 945378 | A | B | 951861 | A | A |
| 945379 | B | C | 909207 | A | A |
| 945380 | D | D | 943487 | A | A |
| 945381 | D | D | 945718 | A | A |
| 948549 | A | A | 947255 | A | A |
| 948550 | A | B | 947260 | A | A |
| 948551 | A | B | 947264 | A | A |
| 948552 | B | D | 947461 | A | A |
| 806940 | D | D | 947476 | A | A |
| 801406 | C | D | 951714 | A | A |
| 811512 | D | D | 951723 | A | A |
| 862028 | B | C | 951863 | A | A |
| 823130 | A | B | 877295 | D | D |
| 825755 | A | B | 912168 | D | D |
| 826790 | A | A | 913172 | D | D |
| 826791 | B | C | 948157 | D | D |
| 857336 | A | B | 926861 | A | A |
| 857362 | D | D | 947306 | A | A |
| 870263 | B | D | 948037 | A | A |
| 883867 | D | D | 948437 | A | A |
| 883979 | A | A | 948439 | A | A |
| 883980 | A | A | 924301 | C | D |
| 883981 | D | D | 912217 | C | D |
| 883982 | D | D | 897755 | C | C |
| 897749 | B | D | 947301 | B | C |
| 911956 | A | B | 897756 | B | B |
| 912005 | B | C | 947732 | B | C |
| 912006 | D | D | 943958 | B | C |
| 912096 | A | B | 943956 | B | C |
| 912097 | B | D | 948222 | B | C |
| 912098 | A | B | 913002 | B | C |
| 912099 | D | D | 948217 | B | C |
| 912205 | D | D | 911083 | B | C |
| 912206 | A | B | 907326 | B | C |
| 912622 | B | C | 907327 | B | C |
| 912668 | D | D | 917129 | B | B |
| 913004 | D | D | 948320 | B | C |
| 913006 | A | A | 948039 | B | C |
| 913178 | B | C | 913056 | B | C |
| 913225 | B | C | 924306 | B | B |
| 913357 | B | C | 926823 | B | C |
| 913439 | A | B | 913138 | B | B |
| 914308 | A | A | 911973 | B | B |
| 914344 | B | D | 913139 | B | B |
| 914930 | A | C | 948036 | B | B |
| 914971 | A | B | 948221 | B | B |
| 917211 | A | B | 926872 | B | B |
| 917282 | A | B | 917128 | B | B |
| 917283 | D | D | 947736 | A | B |
| 917284 | B | C | 947284 | A | B |
| 919318 | A | B | 947294 | A | B |
| 926212 | A | C | 883977 | A | B |
| 926299 | C | D | 945407 | A | B |
| 926300 | A | B | 947274 | A | B |
| 926302 | C | D | 948156 | A | B |
| 927325 | A | B | 947285 | A | B |
| 927326 | B | D | 947287 | A | B |
| 927389 | A | A | 948040 | A | B |
| 927390 | B | D | 944075 | A | B |
| 929474 | A | B | 947839 | A | B |
| 929475 | D | D | 947270 | A | B |
| 943371 | A | C | 947267 | A | B |
| 943496 | A | C | 947308 | A | B |
| 943912 | A | C | 947265 | A | B |
| 943959 | A | B | 947304 | A | B |
| 943960 | A | A | 948319 | A | B |
| 943961 | A | A | 947271 | A | B |
| 943962 | A | B | 947268 | A | A |
| 943963 | A | A | 947282 | A | B |
| 943964 | A | A | 917232 | A | A |
| 943965 | B | D | 947291 | A | B |
| 943966 | A | B | 948438 | A | A |
| 943967 | D | D | 947278 | A | A |
| 944020 | A | A | 947299 | A | A |
| 944021 | A | B | 913057 | A | A |
| 944022 | A | B | 943957 | A | A |
| 944027 | A | B | 947276 | A | A |
| 944028 | A | B | 893312 | A | A |
| 944030 | A | A | 947302 | A | A |
| 944071 | C | D | 947486 | A | A |
| 944074 | A | B | 947289 | A | A |
| 945361 | A | B | 947297 | A | A |
| 945362 | C | D | 947277 | A | A |
| 945363 | C | D | 947283 | A | A |
| 945364 | D | D | 947292 | A | A |
| 945382 | A | B | 947307 | A | A |
| 945383 | A | B | 947487 | A | A |
| 945384 | C | D | 947303 | A | A |
| 945385 | D | D | 947300 | A | A |
| 945459 | A | A | 948219 | A | A |
| 945460 | A | A | 917231 | A | A |
| 945461 | A | A | 947273 | A | A |
| 945462 | A | A | 947286 | A | A |
| 945464 | A | A | 947290 | A | A |
| 945465 | A | A | 947293 | A | A |
| 945470 | A | A | 947298 | A | A |
| 945480 | C | D | 926808 | A | A |
| 945597 | A | A | 947266 | A | A |
| 945598 | A | A | 947272 | A | A |
| 945599 | A | A | 947279 | A | A |
| 945602 | C | D | 947296 | A | A |
| 945603 | A | B | 874477 | A | A |
| 945604 | A | A | 943955 | A | A |
| 945608 | A | B | 947280 | A | A |
| 945611 | D | D | 947295 | A | A |
| 945614 | A | B | 947269 | A | A |
| 945617 | C | D | 947281 | A | A |
| 945621 | C | D | 947733 | A | A |
| 945633 | D | D | 947735 | A | A |
| 945634 | C | D | 874534 | A | A |
| 945635 | A | B | 947275 | A | A |
| 945637 | A | B | 947288 | A | A |
| 945722 | A | A | 947305 | A | A |
| 945723 | B | C | 948038 | A | A |
| 945727 | D | D | 913058 | A | A |
| 945731 | A | A | 947485 | A | A |
| 945920 | A | B | 948215 | A | A |
| 945921 | C | D | 948223 | A | A |
| 945922 | A | B | 948440 | A | A |
| 945923 | D | D | 947838 | A | A |
| 945993 | A | B | 943898 | A | A |
| 945994 | A | B | 943896 | A | A |
| 945996 | B | C | 947835 | A | B |
| 945999 | A | A | 947836 | A | B |
| 946001 | A | A | 943959 | A | B |
| 946004 | B | D | 947837 | A | A |
| 947332 | A | B | 943963 | A | A |
| 947395 | A | A | 943960 | A | A |

A: ≤0.100 μM;
B: >0.100 = 1.000 μM;
C: >1.000-5.000 μM;
D: >5.000 μM;
E >1.670 μM;
F >1.700 μM

TABLE 17

| | GSK3-A | GSK3-B | | GSK3-A | GSK3-B |
|---|---|---|---|---|---|
| | (TR-FRET | (TR-FRET | | (TR-FRET | (TR-FRET |
| | 1 mM ATP); | 1 mM ATP); | | 1 mM ATP); | 1 mM ATP); |
| Compound | GMean; IC₅₀ | GMean; IC₅₀ | Compound | GMean; IC₅₀ | GMean; IC₅₀ |
| number | (μM) | (μM) | number | (μM) | (μM) |
| 949010 | A | A | 945997 | B | C |
| 945713 | A | A | 951714 | B | B |
| 947867 | A | A | 911975 | B | C |
| 947324 | A | A | 948215 | B | B |
| 947329 | A | A | 948778 | B | C |
| 948546 | A | B | 951861 | B | B |
| 913032 | A | A | 947281 | B | B |
| 819575 | A | A | 943481 | B | B |
| 950563 | A | A | 945672 | B | B |
| 948185 | A | A | 948547 | B | C |
| 948307 | A | A | 874510 | B | B |
| 945710 | A | A | 947275 | B | B |
| 947814 | A | A | 948371 | B | B |
| 947859 | A | A | 949177 | B | C |
| 947511 | A | A | 948223 | B | B |
| 819574 | A | A | 943960 | B | B |
| 948418 | A | A | 943960 | B | B |
| 947819 | A | A | 947263 | B | C |
| 811565 | A | A | 914308 | B | C |
| 816800 | A | A | 901468 | B | C |
| 913035 | A | B | 948549 | B | C |
| 913043 | A | B | 949103 | B | B |
| 949917 | A | A | 959343 | B | C |
| 948421 | A | A | 947395 | B | B |
| 945527 | A | B | 948359 | B | C |
| 947333 | A | B | 943964 | B | C |
| 947372 | A | A | 947611 | B | B |
| 949181 | A | B | 948205 | B | B |
| 945704 | A | B | 952759 | B | C |
| 949175 | A | A | 950824 | B | B |
| 951607 | A | B | 948120 | B | C |
| 949336 | A | B | 947253 | B | C |
| 947404 | A | B | 810373 | B | C |
| 948314 | A | A | 948998 | B | C |
| 948419 | A | A | 949650 | B | C |
| 947326 | A | A | 901469 | B | B |
| 950497 | A | B | 945724 | B | C |
| 947808 | A | B | 792340 | B | B |
| 947811 | A | A | 943485 | B | C |
| 951740 | A | B | 947408 | B | C |
| 948037 | A | A | 893311 | B | C |
| 917293 | A | A | 927347 | B | C |
| 943913 | A | B | 883980 | B | C |
| 947384 | A | B | 917088 | B | C |
| 911992 | A | B | 952410 | B | C |
| 913128 | A | B | 943994 | B | C |
| 945725 | A | B | 949401 | B | C |
| 945529 | A | B | 943488 | B | B |
| 909206 | A | B | 951863 | B | B |
| 945481 | A | B | 949324 | B | C |
| 950055 | A | B | 943484 | B | C |
| 913127 | A | B | 947243 | B | C |
| 947363 | A | B | 948812 | B | C |
| 926861 | A | A | 947269 | B | B |
| 945531 | A | B | 917086 | B | C |
| 948771 | A | B | 947414 | B | C |
| 913025 | A | B | 948060 | B | C |
| 810366 | A | B | 874530 | B | C |
| 946099 | A | B | 949169 | B | C |
| 913047 | A | B | 874477 | B | C |
| 913036 | A | B | 917139 | B | B |
| 947860 | A | B | 945543 | B | C |
| 913129 | A | B | 945905 | B | B |
| 947658 | A | B | 947462 | B | C |
| 945718 | A | B | 865193 | B | C |
| 816885 | A | A | 945995 | B | C |
| 950494 | A | B | 952560 | B | D |
| 949183 | A | B | 943482 | B | B |
| 948437 | A | A | 948525 | B | C |
| 947403 | A | B | 949521 | B | C |
| 948150 | A | B | 948358 | B | C |
| 911990 | A | B | 950059 | B | C |
| 810466 | A | B | 826790 | B | C |
| 951948 | A | B | 929454 | B | C |
| 913052 | A | B | 949352 | B | C |
| 913023 | A | B | 951701 | B | C |
| 945328 | A | B | 801413 | B | C |
| 884117 | A | B | 901471 | B | C |
| 826718 | A | B | 949115 | B | C |
| 944033 | A | B | 948828 | B | C |
| 917291 | A | B | 810450 | B | C |
| 913135 | A | B | 947613 | B | C |
| 911981 | A | B | 945670 | B | C |
| 911980 | A | B | 947616 | B | C |
| 909207 | A | B | 949651 | B | D |
| 943990 | A | B | 948184 | B | C |
| 947651 | A | B | 948058 | B | C |
| 945668 | A | B | 945606 | B | C |
| 947861 | A | B | 945613 | B | C |
| 801308 | A | B | 901467 | B | C |
| 945546 | A | B | 790733 | B | C |
| 947306 | A | A | 951725 | B | C |
| 947862 | A | B | 949397 | B | C |
| 951606 | A | B | 947650 | B | D |
| 949185 | A | C | 835315 | B | C |
| 945528 | A | B | 927389 | B | C |
| 943518 | A | B | 945470 | B | C |
| 949648 | A | C | 943542 | B | C |
| 948439 | A | A | 949174 | B | B |
| 947877 | A | B | 913018 | B | C |
| 947255 | A | B | 952021 | B | D |
| 948545 | A | B | 947401 | B | D |
| 947405 | A | B | 948361 | B | C |
| 889665 | A | B | 951612 | B | C |
| 901465 | A | B | 949331 | B | C |
| 949005 | A | C | 865169 | B | D |
| 913058 | A | B | 913006 | B | D |
| 947260 | A | B | 948219 | B | C |
| 943996 | A | B | 911988 | B | D |
| 945712 | A | B | 951949 | B | D |
| 801311 | A | B | 947432 | B | D |
| 913350 | A | B | 949006 | B | D |
| 949919 | A | B | 837683 | B | D |
| 946001 | A | B | 947652 | B | D |
| 949171 | A | C | 949310 | B | D |
| 913045 | A | B | 948529 | B | C |
| 949743 | A | B | 951703 | B | C |
| 948022 | A | B | 948379 | B | D |
| 883873 | A | B | 945657 | B | C |
| 943487 | A | B | 819577 | B | D |
| 947242 | A | B | 948815 | B | D |
| 948118 | A | B | 945920 | B | C |
| 947264 | A | B | 957511 | B | D |
| 945532 | A | B | 946104 | B | D |
| 917150 | A | B | 943541 | B | C |
| 801314 | A | B | 952212 | B | D |
| 947246 | A | B | 948793 | B | D |
| 889639 | A | B | 823721 | B | D |
| 929447 | A | B | 950163 | B | D |
| 913050 | A | B | 927325 | B | D |
| 948038 | A | B | 945614 | B | C |
| 913123 | A | B | 951702 | B | D |
| 947254 | A | B | 943531 | B | D |
| 948142 | A | B | 951788 | B | C |
| 950252 | A | C | 951864 | B | C |
| 947876 | A | B | 957506 | B | D |
| 946089 | A | B | 944074 | B | D |
| 929437 | A | B | 926300 | B | D |
| 948515 | A | B | 893323 | B | D |
| 912171 | A | B | 929388 | B | D |
| 948775 | A | B | 952910 | B | D |
| 945656 | A | B | 951713 | B | C |
| 883874 | A | B | 947410 | B | D |
| 943516 | A | C | 819638 | B | D |
| 837646 | A | C | 943961 | B | D |
| 946093 | A | B | 943961 | B | D |
| 909204 | A | B | 945476 | B | D |
| 893310 | A | B | 947406 | B | D |
| 947464 | A | B | 949761 | B | D |
| 951723 | A | B | 952436 | C | C |
| 927346 | A | C | 951726 | C | C |
| 947869 | A | C | 945607 | C | D |
| 951787 | A | B | 950828 | C | D |
| 952025 | A | C | 959341 | C | D |
| 942642 | A | C | 945475 | C | D |
| 957507 | A | C | 918285 | C | D |
| 949172 | B | C | 789560 | C | D |
| 901470 | B | B | 952565 | C | D |
| 952564 | B | C | 957505 | C | D |
| 826717 | B | C | 957200 | D | D |
| 948313 | B | C | 946103 | D | D |
| 949422 | B | C | 957504 | | D |
| 947288 | B | B | 837753 | D | D |
| 948123 | B | B | 837803 | D | D |
| 948121 | B | B | 913046 | D | D |
| 949649 | B | C | 913311 | D | D |
| 884116 | B | B | 917289 | D | D |
| 949757 | B | C | 917292 | D | D |
| 913030 | B | B | 945471 | D | D |
| 948512 | B | C | 945482 | D | D |
| 944032 | B | C | 947499 | D | D |
| 790754 | B | C | 957197 | D | D |
| 951789 | B | B | 949108 | D | D |
| 947336 | B | C | 949112 | D | D |
| 811557 | B | C | 952566 | D | D |
| 874534 | B | B | 108005 | B | B |
| 108006 | D | D | | | |
| 108791 | C | B | 126193 | | D |
| 108794 | D | D | 126366 | C | B |
| 108954 | C | B | 126367 | D | D |
| 108956 | D | C | 126393 | C | B |
| 114531 | D | C | 126394 | D | |
| 114532 | | D | 126395 | C | B |
| 114679 | A | B | 126396 | D | D |
| 114687 | B | A | 126397 | C | A |
| 114744 | D | D | 126398 | | D |
| 117092 | D | D | 126399 | | D |
| 117093 | C | B | 126400 | C | B |
| 119047 | C | B | 127429 | D | C |
| 119048 | C | B | 127430 | C | B |
| 125976 | C | B | 90285 | D | C |
| 125977 | | D | 90330 | D | D |
| 125978 | C | B | 90709 | | D |
| 125979 | D | C | 90991 | | D |
| 126012 | C | B | 92315 | D | D |
| 126013 | | D | 92737 | D | C |
| 126014 | B | B | 93429 | D | C |
| 126015 | D | D | 107828 | A | B |
| 126190 | C | B | 107829 | C | B |
| 126191 | D | B | 109152 | B | B |

A: ≤0.100 μM;
B: >0.100 = 1.000 μM;
C: >1.000-5.000 μM;
D: >5.000 μM;
E >1.670 μM;
F >1.700 μM

TABLE 18

| | GSK3b (10 | GSK3b (100 | GSK3a (10 | GSK3a (100 |
|---|---|---|---|---|
| | μM ATP) | μM ATP) | μM ATP) | μM ATP) |
| | Average | Average | Average | Average |
| Compound | IC₅₀ | IC₅₀ | IC₅₀ | IC₅₀ |
| number | (μM) | (μM) | (μM) | (μM) |
| 90285 | 0.003 | 0.055 | 0.051 | 0.956 |
| 90709 | 0.038 | 1.738 | 1.791 | 13.719 |
| 90991 | 0.113 | 3.403 | 3.012 | 17.541 |
| 92737 | | 0.066 | | 0.438 |
| 93429 | | 0.134 | | 0.36 |

### Example 7. Additional Physical, Chemical, Biochemical, and Biological Assays of Exemplary Compounds

### Microsomal Stability (MIC)

Stability of compound in liver microsomes was tested in duplicate by incubating compound at 1 μM for 60 minutes at 37° C. Compound level at 60 minutes was compared to level at 0 minutes, and percent remaining was calculated. Assay Parameters

Available species: human and mouse (CD-1). Compound requirements: 5 μL of a 10 mM DMSO solution. Incubation time: 60 minutes at 37° C. Test Concentration: 1 μM.

### Assay Controls

Microsomes with NADPH without compound-matrix interference. Microsomes plus compound without NADPH—non-enzymatic instability.

### MDR1/MDCK Assay

The MDR1/MDCK assay is run at Absorption Systems. This assay is used to determine the blood-brain barrier (BBB) penetration potential of a test compound using MDR1-MDCK cell monolayers. Catalog number EA203. Exemplary results are shown in Table 19.

### Deliverables

The percent recovery of the test compound from the Transwell® wells containing MDR1-MDCK cell monolayers
The apparent permeability (Pₐₚₚ) in both directions
The efflux ratio (Pₐₚₚ B to A)/(Pₐₚₚ A to B)
The blood-brain barrier penetration potential classification:
   High when
      Pₐₚₚ A to B≥3.0×10⁻⁶ cm/s, and efflux <3.0
   Moderate when
      Pₐₚₚ A to B≥3.0×10⁻⁶ cm/s, and 10>efflux≥3.0
   Low when either
      Pₐₚₚ A to B≥3.0×10⁻⁶ cm/s, and efflux≥10, or when
      · Pₐₚₚ A to B<3.0×10⁻⁶ cm/s.

### Substrate

Test compound at 5 μM in HBSSg with maximum DMSO concentration not greater than 1%.

### Assay System

Confluent monolayers of MDR1-MDCK cells, 7 to 11 days old.

### Assay Conditions

Receiver well with 1% BSA in modified Hanks buffer (HBSSg)
Apical and basolateral side at pH 7.4
Dose two monolayers in each direction (N=2)
Dose apical side for (A to B) assessment
Dose basolateral side for (B to A) assessment
Sample both apical and basolateral sides at 120 minutes
Determine the concentrations of test compound using a generic LC-MS/MS method with a minimum 4 point calibration curve.

### Assay QC

The quality of the cell monolayer batch is verified using control compounds before monolayers are released for use
The quality of each monolayer used in the assay is verified by a TEER measurement and by calculating the Pₐₚₚ for a control compound.

TABLE 19

| | | | | | MDCK |
|---|---|---|---|---|---|
| | | | MDCK | MDCK | permeability |
| | Microsome | Microsome | permeability | permeability | (MDRI-MDCK); |
| | stability | stability | (MDRI-MDCK); | (MDR1-MDCK); | Mean; Pₐₚₚ |
| | (human); in | (mouse); in | Mean; Pₐₚₚ | Mean; Pₐₚₚ | Ratio |
| Compound | vitro T_{1/2} | vitro T_{1/2} | (A − B) | (B − A) | (B − A/A − B) |
| number | (min) | (min) | (10⁻⁶ cm/s) | (10⁻⁶ cm/s) | (efflux ratio) |
| 108005 | | 0.69 | 0.1 | 16.31 | 167.3 |
| | | | 1.21 | 6.17 | 5.1 |
| 108954 | | 8.59 | 9.84 | 6.28 | 0.6 |
| | | | 1.98 | 21.22 | 10.7 |
| 114687 | 12.3 | 6.4 | | | |
| 119048 | 1.4 | 0.5 | 1.65 | 70 | 42.3 |
| 125976 | 26.2 | 36 | 0.2 | 61.2 | 301 |
| 125978 | 0.6 | 0.6 | 10.8 | 81.2 | 7.49 |
| 126012 | | | 0.47 | 56.2 | 120 |
| 126014 | | | 0.17 | 32.1 | 187 |
| 126190 | | | 0.08 | 15.7 | 199 |
| 126366 | | | 0.43 | 60 | 138 |
| 126393 | | | 0.59 | 54.5 | 92.5 |
| 126395 | | | 6.48 | 57.6 | 8.9 |
| 126397 | 104.9 | >145 | | | |
| 126400 | | | 0.41 | 48.9 | 119 |
| 127430 | >145 | 74.5 | | | |
| 90285 | 9.978 | 7.685 | 1.6843 | 21.698 | 12.883 |
| | | | 1.37 | 30.24 | 22.14 |
| | | | 13.849 | 9.3765 | 0.6771 |
| | | | 14.94 | 11.71 | 0.78 |
| 90709 | 36.014 | 8.364 | 16.725 | 18.114 | 1.083 |
| | | | 7.5106 | 27.86 | 3.7095 |
| 90991 | >145 | 61.984 | 0.3759 | 8.8128 | 23.447 |
| | | | 0.7265 | 1.3224 | 1.8201 |
| 92737 | 2.27 | | 1.79 | 24.75 | 13.8 |
| | | | 14.41 | 6.52 | 0.45 |
| 93429 | | | | | |
| 107828 | | 6.49 | 5.55 | 7.24 | 1.3 |
| | | | 1.08 | 23.96 | 22.1 |
| 107829 | 8.877 | 2.084 | 1.28 | 27.89 | 21.84 |
| | | | 14.8 | 8.72 | 0.59 |
| 109152 | | 0.97 | 14.8 | 14.76 | 1 |
| | | | 1.79 | 35.64 | 19.9 |

### REFERENCES

1. Bantscheff M, Eberhard D, Abraham Y, Bastuck S, Boesche M, Hobson S, Mathieson T, Perrin J, Raida M, Rau C, Reader V, Sweetman G, Bauer A, Bouwmeester T, Hopf C, Kruse U, Neubauer G, Ramsden N, Rick J, Kuster B, Drewes G. Quantitative chemical proteomics reveals mechanisms of action of clinical ABL kinase inhibitors. Nat Biotechnol. 2007 September; 25(9):1035-44. doi: 10.1038/nbt1328. Epub 2007 Aug. 26. PMID: 17721511.
2. Behrens J, Jerchow B A, Würtele M, Grimm J, Asbrand C, Wirtz R, Kühl M, Wedlich D, Birchmeier W. Functional interaction of an axin homolog, conductin, with beta-catenin, APC, and GSK3beta. Science. 1998 Apr. 24; 280 (5363):596-9. doi: 10.1126/science.280.5363.596. PMID: 9554852.
3. Beurel E, Grieco S F, Jope R S. Glycogen synthase kinase-3 (GSK3): regulation, actions, and diseases. Pharmacol Ther. 2015; 148:114-131. doi: 10.1016/j.pharmthera.2014.11.016 4. Bhat R, Xue Y, Berg S, Hellberg S, Ormo M, Nilsson Y, Radesäter A C, Jerning E, Markgren P O, Borgegard T, Nylöf M, Giménez-Cassina A, Hernández F, Lucas J J, Díaz-Nido J, Avila J. Structural insights and biological effects of glycogen synthase kinase 3-specific inhibitor AR-A014418. J Biol Chem. 2003 Nov. 14; 278(46):45937-45. doi: 10.1074/jbc.M306268200. Epub 2003 Aug. 19. PMID: 12928438.
5. Bhat R V, Budd Haeberlein S L, Avila J. Glycogen synthase kinase 3: a drug target for CNS therapies. J Neurochem. 2004 June; 89(6):1313-7. doi: 10.1111/j.1471-4159.2004.02422.x. PMID: 15189333.
6. Cole A R. GSK3 as a Sensor Determining Cell Fate in the Brain. Front Mol Neurosci. 2012 Feb. 9; 5:4. doi: 10.3389/fnmol.2012.00004. PMID: 22363258; PMCID: PMC3275790.
7. Coghlan M P, Culbert A A, Cross D A, Corcoran S L, Yates J W, Pearce N J, Rausch O L, Murphy G J, Carter P S, Roxbee Cox L, Mills D, Brown M J, Haigh D, Ward R W, Smith D G, Murray K J, Reith A D, Holder J C. Selective small molecule inhibitors of glycogen synthase kinase-3 modulate glycogen metabolism and gene transcription. Chem Biol. 2000 October; 7(10):793-803. doi: 10.1016/s1074-5521(00)00025-9. PMID: 11033082.
8. Dajani R, Fraser E, Roe S M, Young N, Good V, Dale T C, Pearl L H. Crystal structure of glycogen synthase kinase 3 beta: structural basis for phosphate-primed substrate specificity and autoinhibition. Cell. 2001 Jun. 15; 105(6):721-32. doi: 10.1016/s0092-8674(01)00374-9. PMID: 11440715.
9. Dajani R, Fraser E, Roe S M, Yeo M, Good V M, Thompson V, Dale T C, Pearl L H. Structural basis for recruitment of glycogen synthase kinase 3beta to the axin-APC scaffold complex. EMBO J. 2003 Feb. 3; 22(3):494-501. doi: 10.1093/emboj/cdg068. PMID: 12554650; PMCID: PMC140752.
10. del Ser T, Steinwachs K C, Gertz H J, Andrés M V, Gómez-Carrillo B, Medina M, Vericat J A, Redondo P, Fleet D, León T. Treatment of Alzheimer's disease with the GSK-3 inhibitor tideglusib: a pilot study. J Alzheimers Dis. 2013; 33(1):205-15. doi: 10.3233/JAD-2012-120805. PMID: 22936007.
11. Dembowy J, Adissu H A, Liu J C, Zacksenhaus E, Woodgett J R. Effect of glycogen synthase kinase-3 inactivation on mouse mammary gland development and oncogenesis. Oncogene. 2015 July; 34(27):3514-26. doi: 10.1038/onc.2014.279. Epub 2014 Sep. 8. PMID: 25195860; PMCID: PMC4490903.
12. Dillon G M, Henderson J L, Bao C, Joyce J A, Calhoun M, Amaral B. King K W, Bajrami B, Rabah D. (2020). Acute inhibition of the CNS-specific kinase TTBK1 significantly lowers tau phosphorylation at several disease relevant sites. PloS One, 15(4).
13. Doble B W, Patel S, Wood G A, Kockeritz L K, Woodgett J R. Functional redundancy of GSK-3alpha and GSK-3beta in Wnt/beta-catenin signaling shown by using an allelic series of embryonic stem cell lines. Dev Cell. 2007 June; 12(6):957-71. doi: 10.1016/j.devcel.2007.04.001. 6 PMID: 17543867; PMCID: PMC4485918.
14. Domoto T, Pyko I V, Furuta T, et al. Glycogen synthase kinase-3β is a pivotal mediator of cancer invasion and resistance to therapy. Cancer Sci. 2016; 107(10):1363-1372. doi: 10.1111/cas.13028
15. Eftekharzadeh B, Daigle J G, Kapinos L E, Coyne A, Schiantarelli J, Carlomagno Y, Cook C, Miller S J, Dujardin S, Amaral A S, Grima J C, Bennett R E, Tepper K, DeTure M, Vanderburg C R, Corjuc B T, DeVos S L, Gonzalez J A, Chew J, Vidensky S, Gage F H, Mertens J, Troncoso J, Mandelkow E, Salvatella X, Lim R Y H, Petrucelli L, Wegmann S, Rothstein J D, Hyman B T. (2018). Tau Protein Disrupts Nucleocytoplasmic Transport in Alzheimer's Disease. Neuron, 99(5), 925-940.
16. Georgievska B, Sandin J, Doherty J, Mörtberg A, Neelissen J, Andersson A, Gruber S, Nilsson Y, Schött P, Arvidsson P I, Hellberg S, Osswald G, Berg S, Fälting J, Bhat R V. AZD1080, a novel GSK3 inhibitor, rescues synaptic plasticity deficits in rodent brain and exhibits peripheral target engagement in humans. J Neurochem. 2013 May; 125(3):446-56. doi: 10.1111/jnc.12203. Epub 2013 Mar. 11. PMID: 23410232.
17. Goedert M, Spillantini M G, Jakes R, Rutherford D, Crowther R A. Multiple isoforms of human microtubule-associated protein tau: sequences and localization in neurofibrillary tangles of Alzheimer's disease. Neuron. 1989 October; 3(4):519-26. doi: 10.1016/0896-6273(89)90210-9. PMID: 2484340.
18. Ittner L M, Ke Y D, Delerue F, Bi M, Gladbach A, van Eersel J, Wölfing H, Chieng B C, Christie M J, Napier I A, Eckert A, Staufenbiel M, Hardeman E, Götz J. (2010). Dendritic function of tau mediates amyloid-beta toxicity in Alzheimer's disease mouse models. Cell, 142(3), 387-397.
19. Halkina T, Henderson J L, Lin E Y, Himmelbauer M K, Jones J H, Nevalainen M, Feng J, King K, Rooney M. Johnson J L, Marcotte D J, Chodaparambil J V, Kumar P R, Patterson T A, Murugan P, Schuman E, Wong L, Hesson T, Lamore S, Bao C, Calhoun M, Certo H, Amaral B, Dillon G M, Gilfillan R, de Turiso F G. Discovery of Potent and Brain-Penetrant Tau Tubulin Kinase 1 (TTBK1) Inhibitors that Lower Tau Phosphorylation In Vivo. J Med Chem. 2021 May 13; 64(9):6358-6380. doi: 10.1021/acs.jmedchem. 1c00382. Epub 2021 May 4. PMID: 33944571.
20. Hanger D P, Noble W. Functional implications of glycogen synthase kinase-3-mediated tau phosphorylation. Int J Alzheimers Dis. 2011; 2011:352805. doi: 10.4061/2011/352805
21. Henriksen E J, Dokken B B. Role of glycogen synthase kinase-3 in insulin resistance and type 2 diabetes. Curr Drug Targets. 2006 November; 7(11):1435-41. doi: 10.2174/1389450110607011435. PMID: 17100583.
22. Hu S, Ueda M, Stetson L, Ignatz-Hoover J, Moreton S, Chakrabarti A, Xia Z, Karan G, de Lima M, Agrawal M K, Wald D N. A Novel Glycogen Synthase Kinase-3 Inhibitor Optimized for Acute Myeloid Leukemia Differentiation Activity. Mol Cancer Ther. 2016 July; 15(7):1485-1494. doi: 10.1158/1535-7163.MCT-15-0566. Epub 2016 May 9. PMID: 27196775; PMCID: PMC4936967.
23. Kaidanovich-Beilin O, Woodgett J R. GSK-3: Functional Insights from Cell Biology and Animal Models. Front Mol Neurosci. 2011 Nov. 16; 4:40. doi: 10.3389/fnmol.2011.00040. PMID: 22110425; PMCID: PMC3217193.
24. Kerkela R, Kockeritz L, Macaulay K, Zhou J, Doble B W, Beahm C, Greytak S, Woulfe K, Trivedi C M, Woodgett J R, Epstein J A, Force T, Huggins G S. Deletion of GSK-3beta in mice leads to hypertrophic cardiomyopathy secondary to cardiomyoblast hyperproliferation. J Clin Invest. 2008 November; 118(11):3609-18. doi: 10.1172/JCI36245. Epub 2008 Oct. 1. PMID: 18830417; PMCID: PMC2556242.
25. Kim, W. Y., Wang, X., Wu, Y., Doble, B. W., Patel, S., Woodgett, J. R., et al. (2009). GSK-3 is a master regulator of neural progenitor homeostasis. Nat. Neurosci. 12, 1390-1397.
26. Koren S A, Hamm M J, Meier S E, Weiss B E, Nation G K, Chishti E A, Arango J P, Chen J, Zhu H, Blalock E M, Abisambra J F. (2019). Tau drives translational selectivity by interacting with ribosomal proteins. Acta Neuropathologica, 137(4), 571-583.
27. Kurnit, K., Kim, G., Fellman, B. et al. CTNNB1 (beta-catenin) mutation identifies low grade, early stage endometrial cancer patients at increased risk of recurrence. Mod Pathol 30, 1032-1041(2017). https://doi.org/10.1038/modpathol.2017.15
28. Leroy K, Boutajangout A, Authelet M, Woodgett J R, Anderton B H, Brion J P. The active form of glycogen synthase kinase-3beta is associated with granulovacuolar degeneration in neurons in Alzheimer's disease. Acta Neuropathol. 2002 February; 103(2):91-9. doi: 10.1007/s004010100435. Epub 2001 Nov. 29. PMID: 11810173.
29. Lo Monte F, Kramer T, Gu J, Brodrecht M, Pilakowski J, Fuertes A, Dominguez J M, Plotkin B, Eldar-Finkelman H, Schmidt B. Structure-based optimization of oxadiazole-based GSK-3 inhibitors. Eur J Med Chem. 2013 March; 61:26-40. doi: 10.1016/j.ejmech. 2012.06.006. Epub 2012 Jun. 12. PMID: 22749643.
30. Llorens-Martin M, Jurado J, Hernández F, Avila J. GSK-3B, a pivotal kinase in Alzheimer disease. Front Mol Neurosci. 2014; 7:46. Published 2014 May 21. doi: 10.3389/fnmol.2014.00046
31. Luna-Muñoz J, García-Sierra F, Falcón V, Menéndez I, Chávez-Macías L, Mena R. Regional conformational change involving phosphorylation of tau protein at the Thr231, precedes the structural change detected by Alz-50 antibody in Alzheimer's disease. J Alzheimers Dis. 2005 September; 8(1):29-41. doi: 10.3233/jad-2005-8104. PMID: 16155347.
32. Ly, P. T., Wu, Y., Zou, H., Wang, R., Zhou, W., Kinoshita, A., et al. (2013). Inhibition of GSK3beta-mediated BACE1 expression reduces Alzheimer-associated phenotypes. J. Clin. Invest. 123, 224-235. doi: 10.1172/JCI64516
33. Ma C, Wang J, Gao Y, Gao T W, Chen G, Bower K A, Odetallah M, Ding M, Ke Z, Luo J. The role of glycogen synthase kinase 3beta in the transformation of epidermal cells. Cancer Res. 2007 Aug. 15; 67(16):7756-64. doi: 10.1158/0008-5472.CAN-06-4665. PMID: 17699780.
34. Martin L, Latypova X, Wilson C M, Magnaudeix A, Perrin M L, Yardin C, Terro F. Tau protein kinases: involvement in Alzheimer's disease. Ageing Res Rev. 2013 January; 12(1):289-309.

doi: 10.1016/j.arr.2012.06.003. Epub 2012 Jun. 26. PMID: 22742992.
35. Matsuoka Y, Okazaki M, Kitamura Y, Taniguchi T. Developmental expression of P-glycoprotein (multidrug resistance gene product) in the rat brain. J Neurobiol. 1999 Jun. 5; 39(3):383-92. doi: 10.1002/(sici) 1097-4695 (19990605) 39:3<383::aid-neu5>3.0.co; 2-4. PMID: 10363911.
36. McCamphill P K, Stoppel L J, Senter R K, Lewis M C, Heynen A J, Stoppel D C, Sridhar V, Collins K A, Shi X, Pan J Q, Madison J, Cottrell J R, Huber K M, Scolnick E M, Holson E B, Wagner F F, Bear M F. Selective inhibition of glycogen synthase kinase 3 corrects pathophysiology in a mouse model of fragile X syndrome. Sci Transl Med. 2020 May 20; 12(544):eaam8572. doi: 10.1126/scitranslmed.aam8572. PMID: 32434848; PMCID: PMC8095719.
37. McCubrey J A, Steelman L S, Bertrand F E, et al. GSK-3 as potential target for therapeutic intervention in cancer. Oncotarget. 2014; 5(10):2881-2911. doi: 10.18632/oncotarget.2037
38. Meijer L, Flajolet M, Greengard P. Pharmacological inhibitors of glycogen synthase kinase 3. Trends Pharmacol Sci. 2004 September; 25(9):471-80. doi: 10.1016/j.tips.2004.07.006. PMID: 15559249.
39. Min W W, Yuskaitis C J, Yan Q, Sikorski C, Chen S, Jope R S, Bauchwitz R P. Elevated glycogen synthase kinase-3 activity in Fragile X mice: key metabolic regulator with evidence for treatment potential. Neuropharmacology. 2009 February; 56(2):463-72. doi: 10.1016/j.neuropharm.2008.09.017. Epub 2008 Oct. 14. PMID: 18952114; PMCID: PMC2707186.
40. Miyoshi Y, Iwao K, Nagasawa Y, Aihara T, Sasaki Y, Imaoka S, Murata M, Shimano T, Nakamura Y. Activation of the beta-catenin gene in primary hepatocellular carcinomas by somatic alterations involving exon 3. Cancer Res. 1998 Jun. 15; 58(12):2524-7. PMID: 9635572.
41. Morgansmith, M., Wu, Y., Zhu, X., Pringle, J., and Snider, W. D. (2014). GSK-3 signaling in developing cortical neurons is essential for radial migration and dendritic orientation. Elife 3, 1057-1066. doi: 10.7554/eLife.02663
42. Neddens, J., Temmel, M., Flunkert, S. et al. Phosphorylation of different tau sites during progression of Alzheimer's disease. acta neuropathol commun 6, 52 (2018). https://doi.org/10.1186/s40478-018-0557-6
43. Palomo V, Perez D I, Perez C, Morales-Garcia J A, Soteras I, Alonso-Gil S, Encinas A, Castro A, Campillo N E, Perez-Castillo A, Gil C, Martinez A. 5-imino-1,2,4-thiadiazoles: first small molecules as substrate competitive inhibitors of glycogen synthase kinase 3. J Med Chem. 2012 Feb. 23; 55(4):1645-61. doi: 10.1021/jm201463v. Epub 2012 Feb. 3. PMID: 22257026.
44. Phiel, C. J., Wilson, C. A., Lee, V. M., and Klein, P. S. (2003). GSK-3alpha regulates production of Alzheimer's disease amyloid-beta peptides. Nature 423, 435-439. doi: 10.1038/nature01640
45. Plattner, F., Angelo, M., and Giese, K. P. (2006). The roles of cyclin-dependent kinase 5 and glycogen synthase kinase 3 in tau hyperphosphorylation. J. Biol. Chem. 281, 25457-25465. doi: 10.1074/jbc.M603469200
46. O'Leary O, Nolan Y. Glycogen synthase kinase-3 as a therapeutic target for cognitive dysfunction in neuropsychiatric disorders. CNS Drugs. 2015 January; 29(1):1-15. doi: 10.1007/s40263-014-0213-z. PMID: 25380674.
47. Racaud-Sultan C, Vergnolle N. GSK3β, a Master Kinase in the Regulation of Adult Stem Cell Behavior. Cells. 2021 Jan. 24; 10(2):225. doi: 10.3390/cells10020225. PMID: 33498808; PMCID: PMC7911451.
48. Reinecke M., Heinzlmeir S., Wilhelm M., Médard G., Klaeger S., Kuster B. Kinobeads: A Chemical Proteomic Approach for Kinase Inhibitor Selectivity Profiling and Target Discovery, Target Discovery and Validation. 2019 October 14^{th}, pp. 97-130, Book Chapter, Wiley-VCH Verlag GmbH & Co. KGaA
49. Reinecke M, Ruprecht B, Poser S, Wiechmann S, Wilhelm M, Heinzlmeir S, Kuster B, Médard G. Chemoproteomic Selectivity Profiling of PIKK and PI3K Kinase Inhibitors. ACS Chem Biol. 2019 Apr. 19; 14(4):655-664. doi: 10.1021/acschembio.8b01020. Epub 2019 Apr. 3. PMID: 30901187.
50. Rodríguez-Martín T, Cuchillo-Ibáñez I, Noble W, Nyenya F, Anderton B H, Hanger D P. Tau phosphorylation affects its axonal transport and degradation. Neurobiol Aging. 2013; 34(9):2146-2157. doi: 10.1016/j.neurobiolaging.2013.03.015
51. Santos J R F, Bauer C, Schuchhardt J, Wedekind D, Waniek K, Lachmann I, Wiltfang J, Vogelgsang J. Validation of a prototype tau Thr231 phosphorylation CSF ELISA as a potential biomarker for Alzheimer's disease. J Neural Transm (Vienna). 2019 March; 126(3):339-348. doi: 10.1007/s00702-019-01982-5. Epub 2019 Feb. 14. PMID: 30767082.
52. Sato N, Meijer L, Skaltsounis L, Greengard P, Brivanlou A H. Maintenance of pluripotency in human and mouse embryonic stem cells through activation of Wnt signaling by a pharmacological GSK-3-specific inhibitor. Nat Med. 2004 January; 10(1):55-63. doi: 10.1038/nm979. Epub 2003 Dec. 21. PMID: 14702635.
53. Soutar, Kim, Williamson et al., “Evidence that glycogen synthase kinase-3 isoforms have distinct substrate preference in the brain,” Journal of Neurochemistry, vol. 115, no. 4, pp. 974-983, 2010.
54. Wagner F F, Benajiba L, Campbell A J, Weïwer M, Sacher J R, Gale J P, Ross L, Puissant A, Alexe G, Conway A, Back M, Pikman Y, Galinsky I, DeAngelo D J, Stone R M, Kaya T, Shi X, Robers M B, Machleidt T, Wilkinson J, Hermine O, Kung A, Stein A J, Lakshminarasimhan D, Hemann M T, Scolnick E, Zhang Y L, Pan J Q, Stegmaier K, Holson E B. Exploiting an Asp-Glu “switch” in glycogen synthase kinase 3 to design paralog-selective inhibitors for use in acute myeloid leukemia. Sci Transl Med. 2018 Mar. 7; 10(431):eaam8460. doi: 10.1126/scitransimed.aam8460. PMID: 29515000; PMCID: PMC6553635.
55. Wang Y, Dou X, Jiang L, Jin H, Zhang L, Zhang L, Liu Z. Discovery of novel glycogen synthase kinase-3a inhibitors: Structure-based virtual screening, preliminary SAR and biological evaluation for treatment of acute myeloid leukemia. Eur J Med Chem. 2019 Jun. 1; 171:221-234. doi: Woodgett, J R “cDNA cloning and properties of glycogen synthase kinase-3,” Methods in 56. Enzymology, vol. 200, pp. 564-577, 1991.
57. Yao H B, Shaw P C, Wong C C, Wan D C. Expression of glycogen synthase kinase-3 isoforms in mouse tissues and their transcription in the brain. J Chem Neuroanat. 2002, May; 23(4):291-7. doi: 10.1016/s0891-0618 (02) 00014-5. PMID: 12048112.
58. Yost C, Torres M, Miller J R, Huang E, Kimelman D, Moon R T. The axis-inducing activity, stability, and subcellular distribution of beta-catenin is regulated in Xenopus embryos by glycogen synthase kinase 3. Genes Dev. 1996 Jun. 15; 10(12):1443-54. doi: 10.1101/gad.10.12.1443. PMID: 8666229.
59. Zhou F Q, Snider W D. GSK-3β and microtubule assembly in axons. Science. 2005; 308(5719):211-214
60. Zhou L, McInnes J, Wierda K, Holt M, Herrmann A G, Jackson R J, Wang Y C, Swerts J, Beyens J, Miskiewicz K, Vilain S, Dewachter I, Moechars D, De Strooper B, Spires-Jones T L, De Wit J, Verstreken P. (2017). Tau association with synaptic vesicles causes presynaptic dysfunction. Nat Commun, 8, 15295.

### Equivalents and Scope

In the claims and throughout, articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the disclosure can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present disclosure, as defined in the following claims.

What is claimed is:

1. A compound, or a pharmaceutically acceptable salt, solvate, hydrate, polymorph, co-crystal, tautomer, stereoisomer, isotopically labeled compound, or prodrug thereof, wherein the compound is of Formula I-A or I-B: 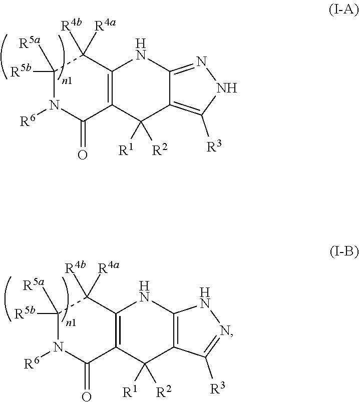
wherein: [symbol]
is a single or double bond;
R¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R² is optionally substituted alkyl, optionally substituted alkenyl, or optionally substituted alkynyl;
or R¹ and R² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
R³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A}) R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)OR^{A}, —S(═O)SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(O)R^{A}, —NR^{A}S(O)OR^{A}, —NR^{A}S(═O)₂SR^{A}, —NR^{A}S(═O)₂N(R^{A})₂, —Si(R^{A})₂, —Si(R^{A})₂OR^{A}, —Si(R^{A})(OR^{A})₂, —Si(OR^{A})₃, —OSi(R^{A})₂, —OSi(R^{A})₂OR^{A}, —OSi(R^{A})(OR^{A})₂, or —OSi(OR^{A})₃;
each of R^{4a} and R^{4b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each instance of R^{A} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{A} attached to the same intervening atom are joined together with the intervening atom to form an optionally substituted, monocyclic, heterocyclic or heteroaryl ring; and
or R^{4a} and R^{4b} are taken together with their intervening atom to form an optionally substituted, monocyclic, carbocyclic or heterocyclic ring;
R⁶ is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl; and
n1 is 0 or 1;
when n1 is 1:
each of R^{5a} and R^{5b} independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl, or R^{5a} and R^{5b} are taken together with their intervening atom to form an optionally substituted, monocyclic, carbocyclic or heterocyclic ring;
or R^{4b} and R^{5a} are taken together with their intervening atom to form an optionally substituted, monocyclic, carbocyclic, heterocyclic, aryl, or heteroaryl ring;
provided that the compound is not of the formula:

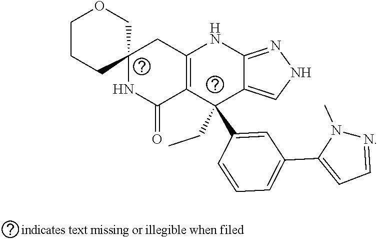 [SMILES: CC[C@]1(c2cccc(-c3ccnn3C)c2)C2=C(C[C@]3(CCCOC3)NC2=O)Nc2n[nH]cc21]

2. The compound of claim 1, or a pharmaceutically acceptable salt, solvate, hydrate, polymorph, co-crystal, tautomer, stereoisomer, isotopically labeled compound, or prodrug thereof, wherein n1 is 0.

3. The compound of claim 1, or a pharmaceutically acceptable salt, solvate, hydrate, polymorph, co-crystal, tautomer, stereoisomer, isotopically labeled compound, or prodrug thereof, wherein n1 is 1.

4. The compound of any one of claims 1-3, or a pharmaceutically acceptable salt, solvate, hydrate, polymorph, co-crystal, tautomer, stereoisomer, isotopically labeled compound, or prodrug thereof, wherein R⁶ is hydrogen.

5. The compound of any one of claims 1-3, or a pharmaceutically acceptable salt, solvate, hydrate, polymorph, co-crystal, tautomer, stereoisomer, isotopically labeled compound, or prodrug thereof, wherein R⁶ is or optionally substituted alkyl.

6. The compound of any one of claims 1-3, or a pharmaceutically acceptable salt, solvate, hydrate, polymorph, co-crystal, tautomer, stereoisomer, isotopically labeled compound, or prodrug thereof, wherein R⁶ is optionally substituted aryl or optionally substituted heteroaryl.

7. The compound of claim 6, or a pharmaceutically acceptable salt, solvate, hydrate, polymorph, co-crystal, tautomer, stereoisomer, isotopically labeled compound, or prodrug thereof, wherein R⁶ is optionally substituted pyridyl, optionally substituted phenyl, or optionally substituted pyrimidinyl.

8. The compound of any one of claims 1-7, or a pharmaceutically acceptable salt, solvate, hydrate, polymorph, co-crystal, tautomer, stereoisomer, isotopically labeled compound, or prodrug thereof, wherein each of R^{5a} and R^{5b} is independently hydrogen, halogen, or optionally substituted alkyl.

9. The compound of claim 8, or a pharmaceutically acceptable salt, solvate, hydrate, polymorph, co-crystal, tautomer, stereoisomer, isotopically labeled compound, or prodrug thereof, wherein R^{5a} and R^{5b} are —CH₃.

10. The compound of claim 8, or a pharmaceutically acceptable salt, solvate, hydrate, polymorph, co-crystal, tautomer, stereoisomer, isotopically labeled compound, or prodrug thereof, wherein R^{5a} and R^{5b} are hydrogen.

11. The compound of any one of claims 1-7, or a pharmaceutically acceptable salt, solvate, hydrate, polymorph, co-crystal, tautomer, stereoisomer, isotopically labeled compound, or prodrug thereof, wherein R^{5a} and R^{5b} are taken together with their intervening atom to form an optionally substituted, monocyclic, carbocyclic ring.

12. The compound of any one of claims 1-7, or a pharmaceutically acceptable salt, solvate, hydrate, polymorph, co-crystal, tautomer, stereoisomer, isotopically labeled compound, or prodrug thereof, wherein R^{5a} and R⁵⁶ are taken together with their intervening atom to form an optionally substituted, monocyclic, heterocyclic ring.

13. The compound of claim 12, or a pharmaceutically acceptable salt, solvate, hydrate, polymorph, co-crystal, tautomer, stereoisomer, isotopically labeled compound, or prodrug thereof, wherein R^{5a} and R^{5b} are taken together with their intervening atom to form an optionally substituted, monocyclic, heterocyclic ring comprising O and/or S heteroatom(s) as the only heteroatoms in the heterocyclic ring.

14. The compound of any one of claims 1-13, or a pharmaceutically acceptable salt, solvate, hydrate, polymorph, co-crystal, tautomer, stereoisomer, isotopically labeled compound, or prodrug thereof, wherein each of R^{4a} and R^{4b} is independently hydrogen, halogen, or optionally substituted alkyl.

15. The compound of any one of claims 1-14, or a pharmaceutically acceptable salt, solvate, hydrate, polymorph, co-crystal, tautomer, stereoisomer, isotopically labeled compound, or prodrug thereof, wherein R¹ is optionally substituted aryl.

16. The compound of claim 15, or a pharmaceutically acceptable salt, solvate, hydrate, thereof, wherein R¹ is 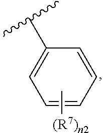
wherein:
each instance of R⁷ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —OC(═O)R^{D}, —OC(═O)OR^{D}, —OC(O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NRDS(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃; and
n2 is 0, 1, 2, 3, 4, or 5.

17. The compound of claim 16, or a pharmaceutically acceptable salt, solvate, hydrate, polymorph, co-crystal, tautomer, stereoisomer, isotopically labeled compound, or prodrug thereof, wherein R¹ is

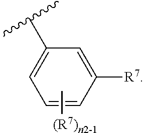

18. The compound of claim 16 or 17, or a pharmaceutically acceptable salt, solvate, hydrate, polymorph, co-crystal, tautomer, stereoisomer, isotopically labeled compound, or prodrug thereof, wherein n2 is 1.

19. The compound of any one of claims 16-18, or a pharmaceutically acceptable salt, solvate, hydrate, polymorph, co-crystal, tautomer, stereoisomer, isotopically labeled compound, or prodrug thereof, wherein R⁷ is C₁₋₄ haloalkyl.

20. The compound of any one of claims 16-18, or a pharmaceutically acceptable salt, solvate, hydrate, polymorph, co-crystal, tautomer, stereoisomer, isotopically labeled compound, or prodrug thereof, wherein R⁷ is optionally substituted heteroaryl comprising one or more N atoms.

21. The compound of claim 20, or a pharmaceutically acceptable salt, solvate, hydrate, polymorph, co-crystal, tautomer, stereoisomer, isotopically labeled compound, or prodrug thereof, wherein R⁷ is optionally substituted pyridinyl or optionally substituted pyrazolyl.

22. The compound of any one of claims 1-21, or a pharmaceutically acceptable salt, solvate, hydrate, polymorph, co-crystal, tautomer, stereoisomer, isotopically labeled compound, or prodrug thereof, wherein R² is optionally substituted alkyl.

23. The compound of claim 22, or a pharmaceutically acceptable salt, solvate, hydrate, polymorph, co-crystal, tautomer, stereoisomer, isotopically labeled compound, or prodrug thereof, wherein R² is unsubstituted C₁-C₄ alkyl.

24. The compound of any one of claims 1-14, or a pharmaceutically acceptable salt, solvate, hydrate, polymorph, co-crystal, tautomer, stereoisomer, isotopically labeled compound, or prodrug thereof, wherein R₁ and R² are taken together with their intervening atom to form an optionally substituted, monocyclic, carbocyclic ring.

25. The compound of any one of claims 1-14, or a pharmaceutically acceptable salt, solvate, hydrate, polymorph, co-crystal, tautomer, stereoisomer, isotopically labeled compound, or prodrug thereof, wherein R₁ and R² are taken together with their intervening atom to form an optionally substituted, monocyclic, heterocyclic ring.

26. The compound of claim 25, or a pharmaceutically acceptable salt, solvate, hydrate, polymorph, co-crystal, tautomer, stereoisomer, isotopically labeled compound, or prodrug thereof, wherein R¹ and R² are taken together with their intervening atom to form an optionally substituted piperidinyl.

27. The compound of any one of claims 1-26, or a pharmaceutically acceptable salt, solvate, prodrug thereof, wherein R³ is hydrogen, optionally substituted C₁-C₆ alkyl, or halogen.

28. The compound of claim 27, or a pharmaceutically acceptable salt, solvate, hydrate, polymorph, co-crystal, tautomer, stereoisomer, isotopically labeled compound, or prodrug thereof, wherein R³ is hydrogen, fluorine, —CH₃, —CH₂F, —CHF₂, or —CF₃.

29. The compound of claim 1, or a pharmaceutically acceptable salt, solvate, hydrate, polymorph, co-crystal, tautomer, stereoisomer, isotopically labeled compound, or prodrug thereof, wherein the compound is any one of the formulae shown in Table 1:
TABLE 1

| | |
|---|---|
| 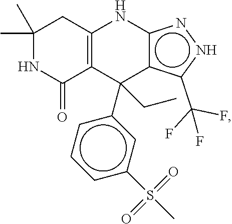 | 947799 |
| 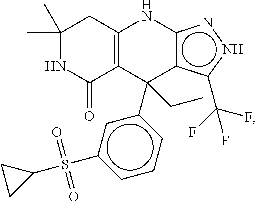 | 947859 |
| 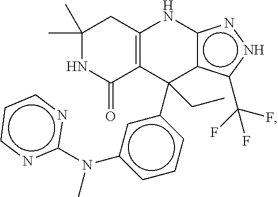 | 948176 |
| 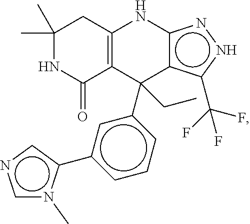 | 948180 |
| 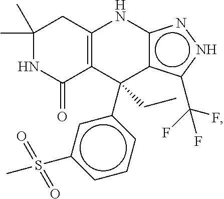 | 948418 |
| 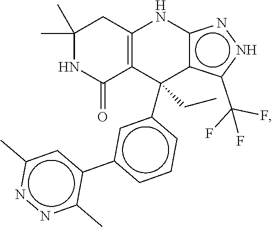 | 949917 |
| 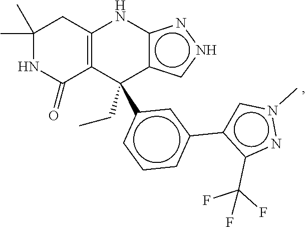 | 950563 |
| 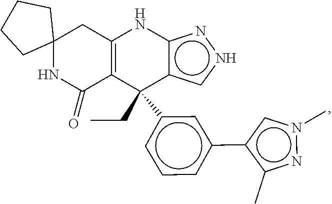 | 950252 |
| 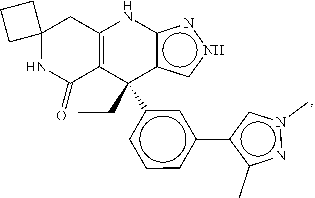 | 950055 |
| 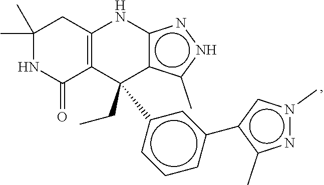 | 946001 |
| 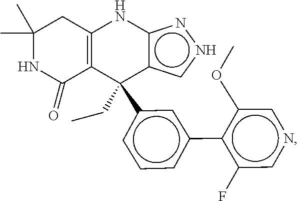 | 948150 |
| 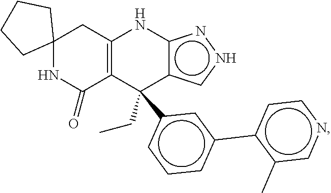 | 948022 |
| 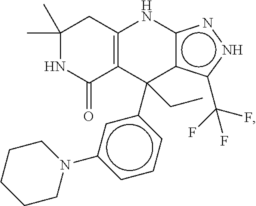 | 947861 |
| 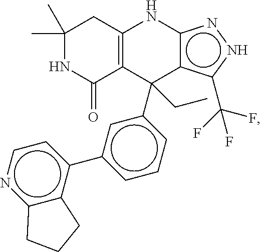 | 947862 |
| 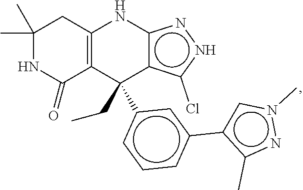 | 949336 |
| 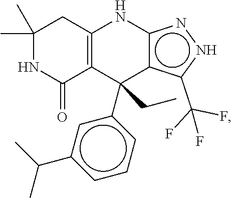 | 950494 |
| 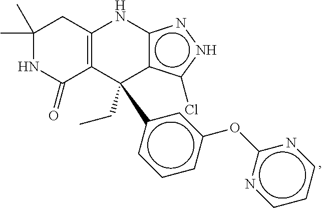 | 951606 |
| 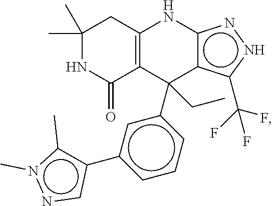 | 947403 |
| 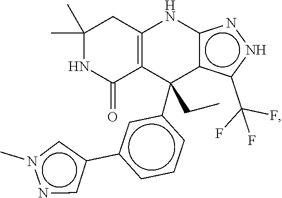 | 947877 |
| 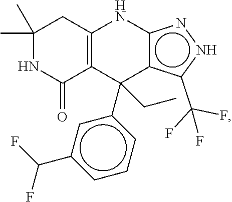 | 948305 |
| 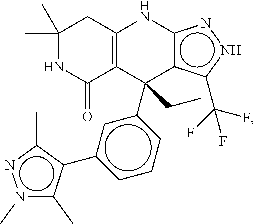 | 948421 |
| 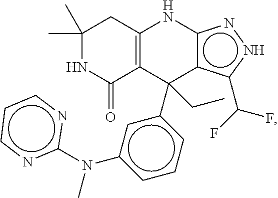 | 950484 |
| 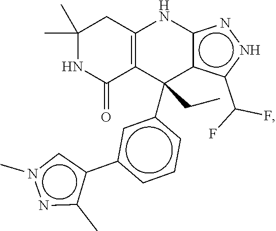 | 950497 |
| 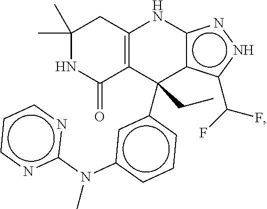 | 951740 |
| 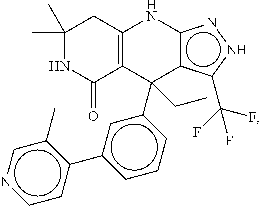 | 947404 |
| 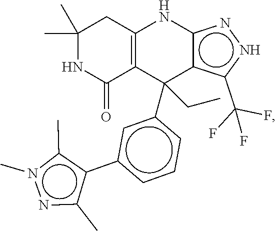 | 947568 |
| 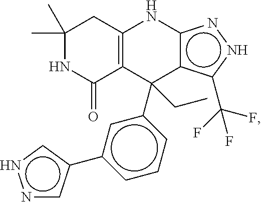 | 947677 |
| 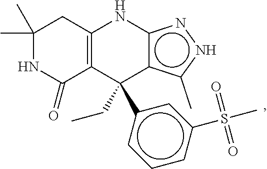 | 947811 |
| 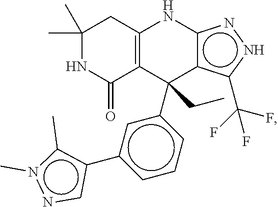 | 947819 |
| 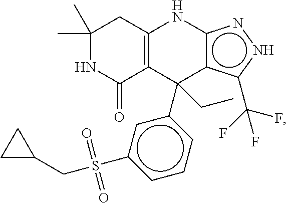 | 948179 |
| 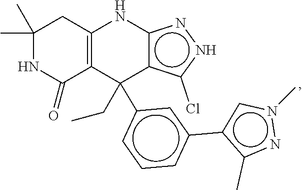 | 948768 |
| 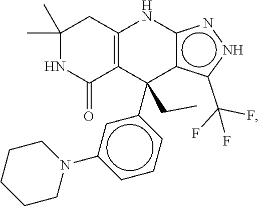 | 949183 |
| 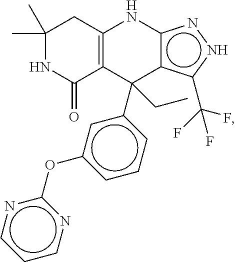 | 950323 |
| 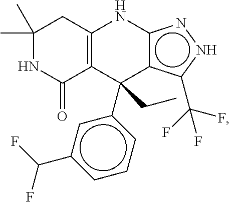 | 951948 |
| 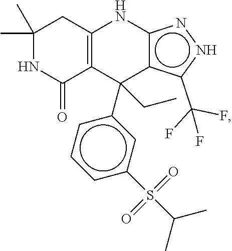 | 947800 |
| 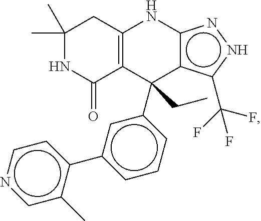 | 947814 |
| 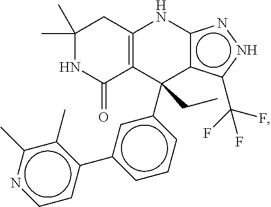 | 948185 |
| 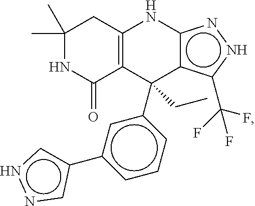 | 948307 |
| 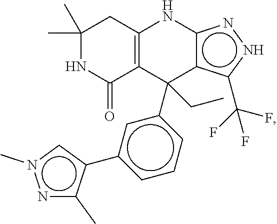 | 948314 |
| 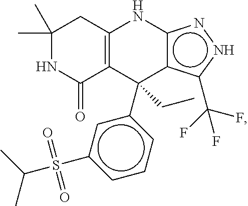 | 948419 |
| 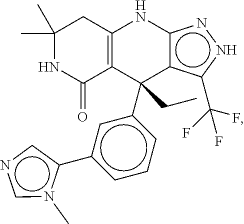 | 949010 |
| 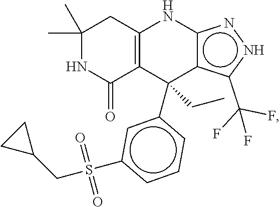 | 949175 |
| 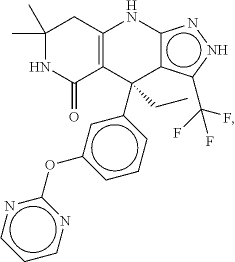 | 951607 |
| 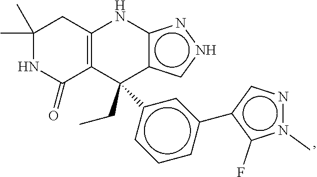 | 949395 |
| 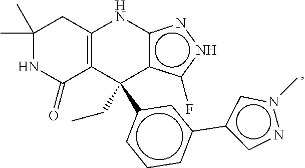 | 949002 |
| 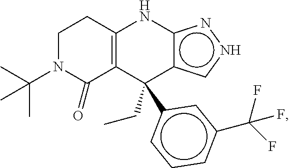 | 945634 |
| 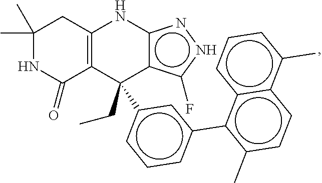 | 949392 |
| 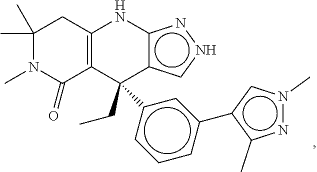 | 949008 |
| 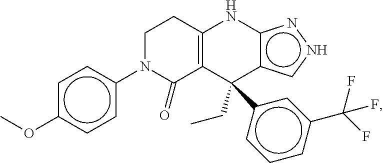 | 948227 |
| 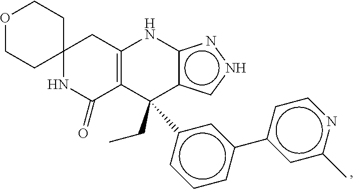 | 948524 |
| 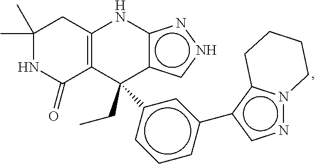 | 947415 |
| 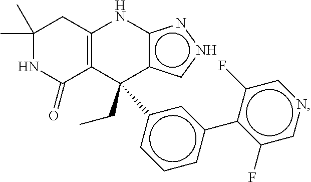 | 948514 |
| 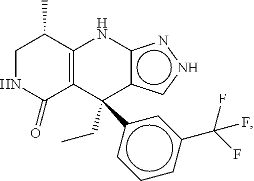 | 945384 |
| 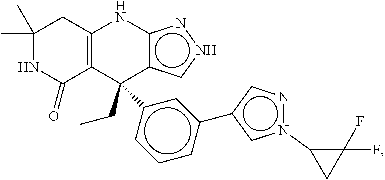 | 949393 |
| 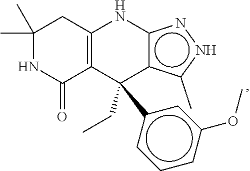 | 949328 |
| 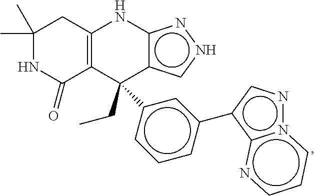 | 949389 |
| 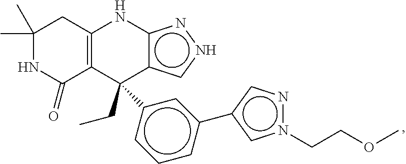 | 949114 |
| 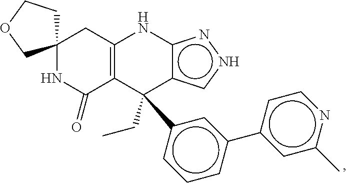 | 948148 |
| 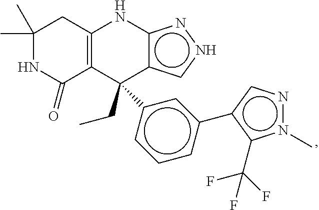 | 948826 |
| 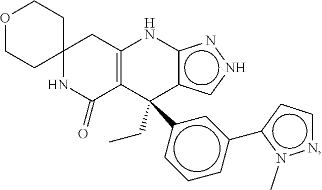 | 948526 |
| 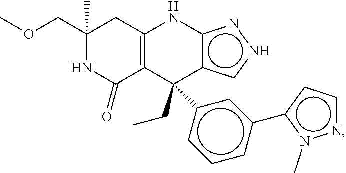 | 947907 |
| 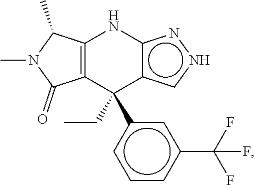 | 913475 |
| 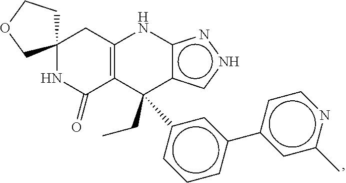 | 948141 |
| 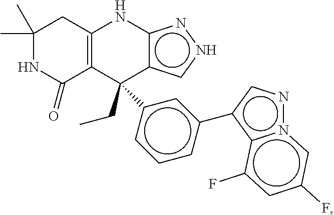 | 949390 |
| 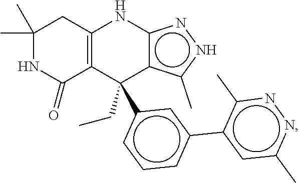 | 949332 |
| 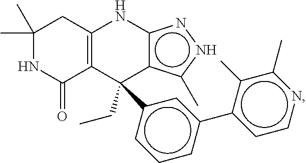 | 948183 |
| 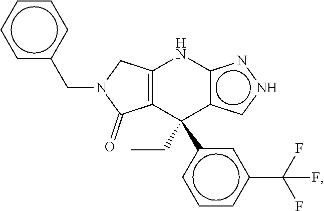 | 929389 |
| 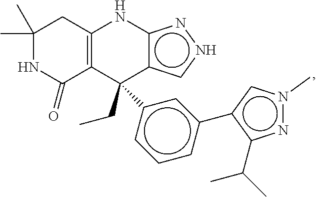 | 949106 |
| 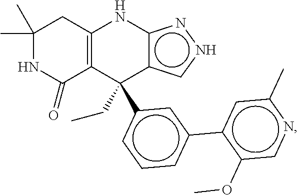 | 949512 |
| 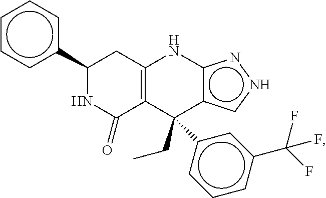 | 945362 |
| 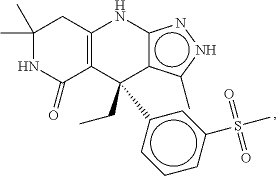 | 947815 |
| 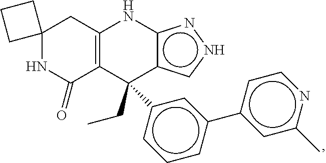 | 949517 |
| 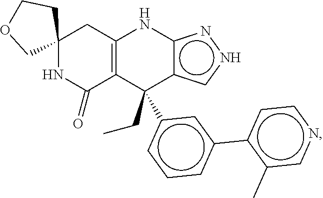 | 948662 |
| 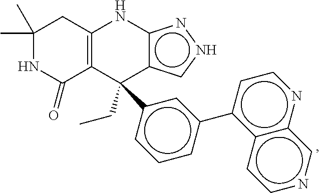 | 949110 |
| 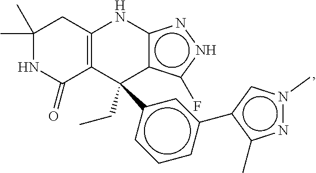 | 949176 |
| 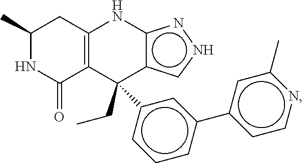 | 926299 |
| 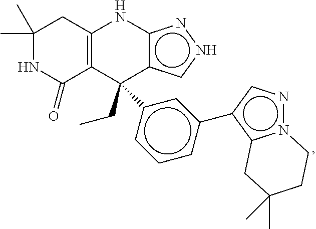 | 949265 |
| 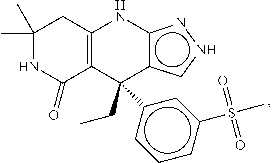 | 945621 |
| 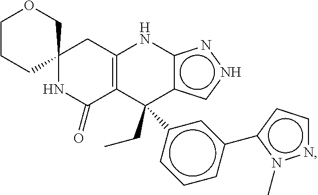 | 948365 |
| 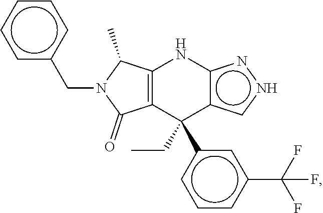 | 930332 |
| 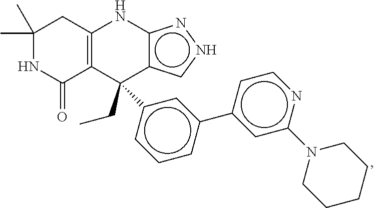 | 948646 |
| 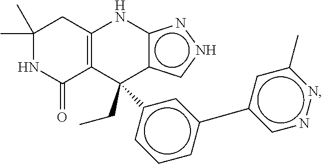 | 944071 |
| 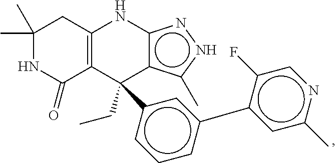 | 949327 |
| 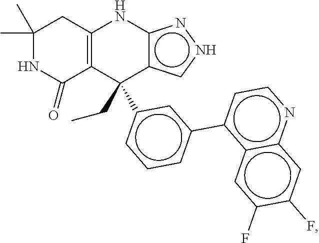 | 948664 |
| 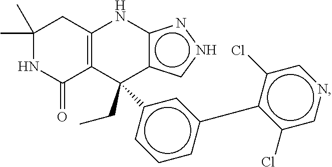 | 948824 |
| 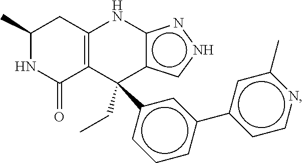 | 926302 |
| 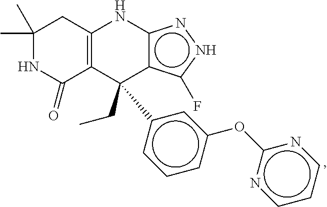 | 951944 |
| 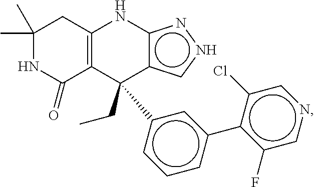 | 948363 |
| 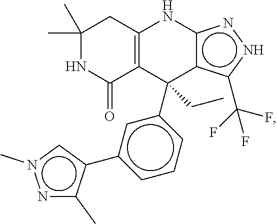 | 948309 |
| 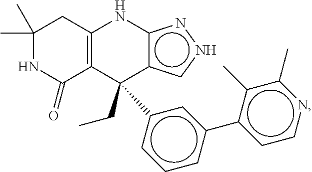 | 948188 |
| 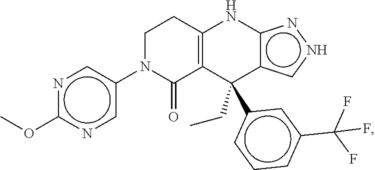 | 949127 |
| 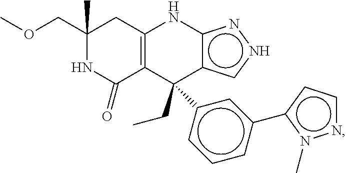 | 947908 |
| 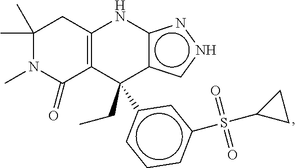 | 949184 |
| 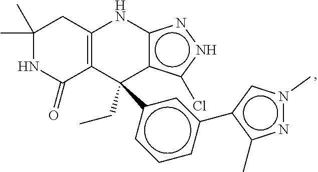 | 949329 |
| 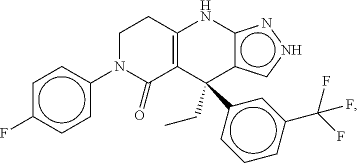 | 948225 |
| 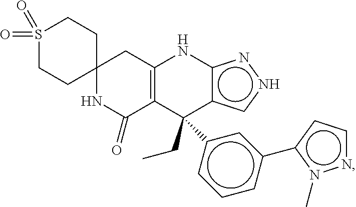 | 949400 |
| 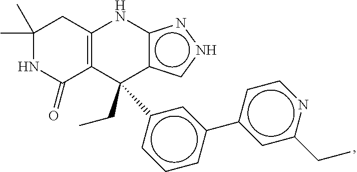 | 948360 |
| 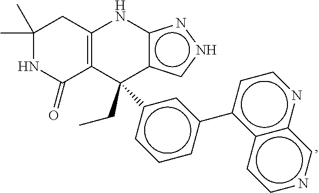 | 948819 |
| 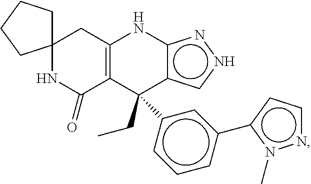 | 948019 |
| 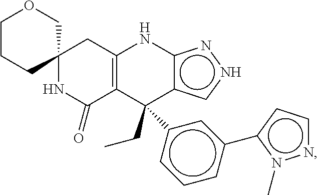 | 948366 |
| 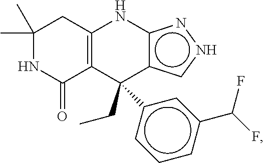 | 949007 |
| 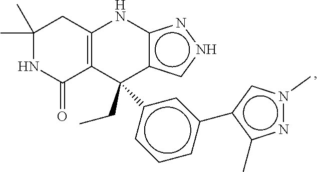 | 945480 |
| 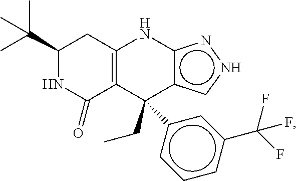 | 945921 |
| 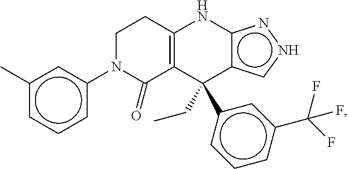 | 949041 |
| 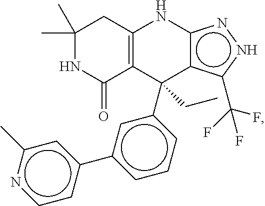 | 948773 |
| 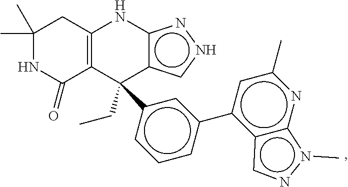 | 952563 |
| 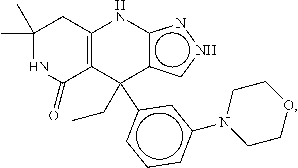 | 945602 |
| 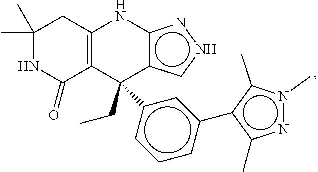 | 947413 |
| 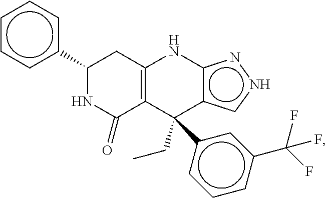 | 945363 |
| 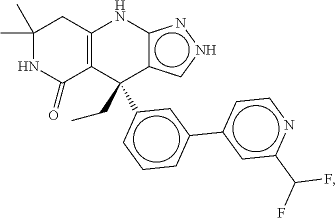 | 949388 |
| 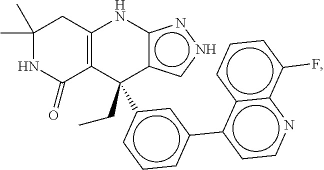 | 948813 |
| 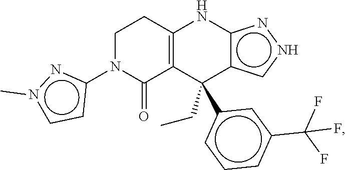 | 949836 |
| 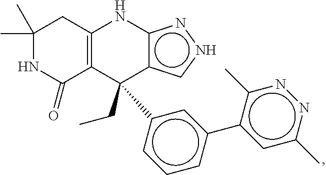 | 949334 |
| 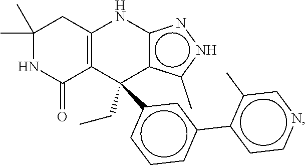 | 947409 |
| 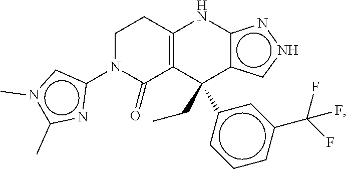 | 952478 |
| 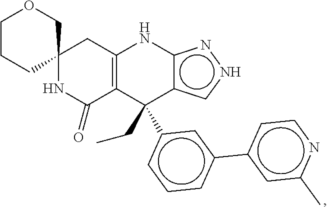 | 948146 |
| 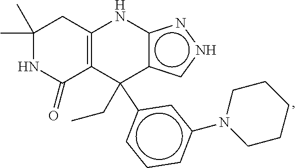 | 947566 |
| 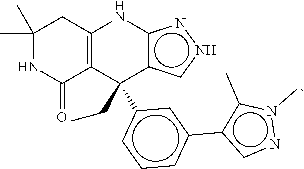 | 945617 |
| 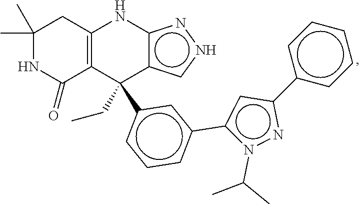 | 952411 |
| 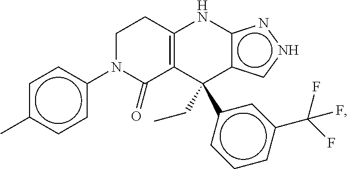 | 949119 |
| 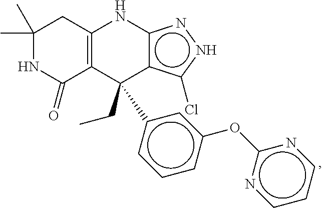 | 951605 |
| 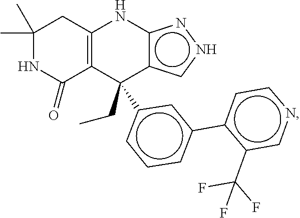 | 949516 |
| 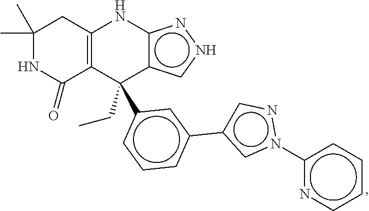 | 952566 |
| 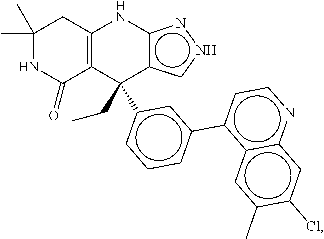 | 948645 |
| 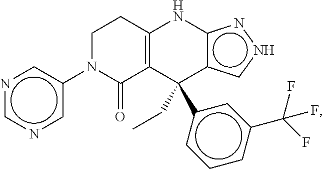 | 948380 |
| 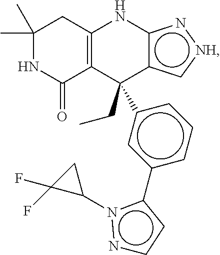 | 949518 |
| 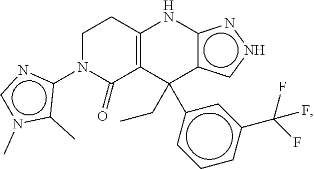 | 951598 |
| 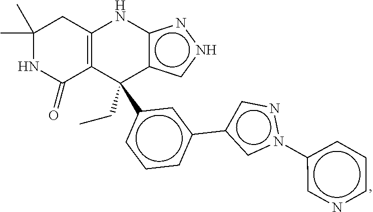 | 952024 |
| 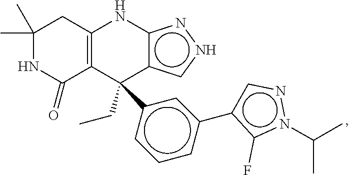 | 950061 |
| 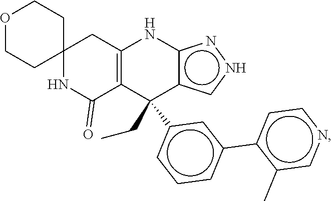 | 948367 |
| 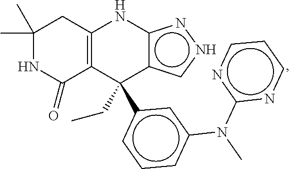 | 949179 |
| 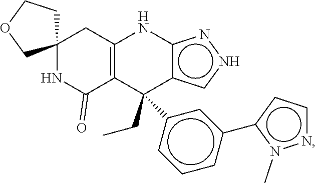 | 948014 |
| 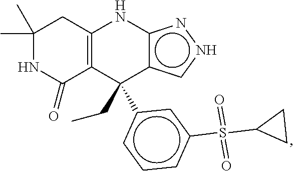 | 946004 |
| 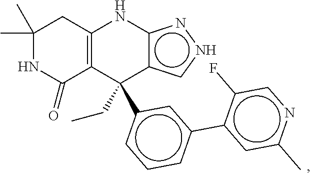 | 947822 |
| 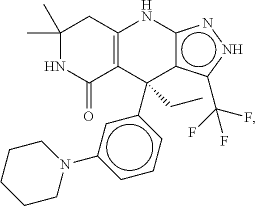 | 949178 |
| 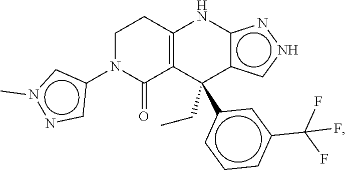 | 949271 |
| 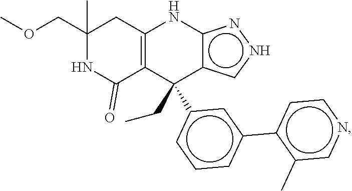 | 948127 |
| 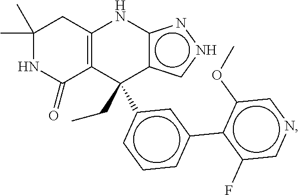 | 948145 |
| 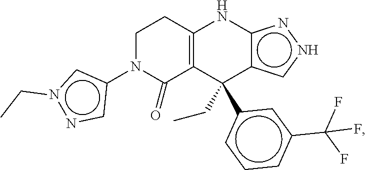 | 949868 |
| 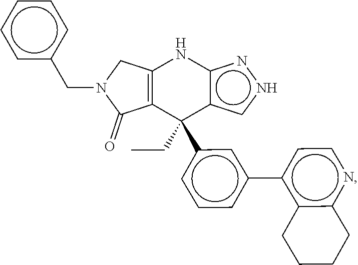 | 948552 |
| 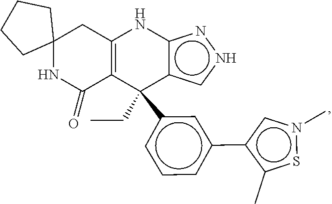 | 950255 |
| 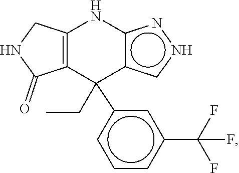 | 913156 |
| 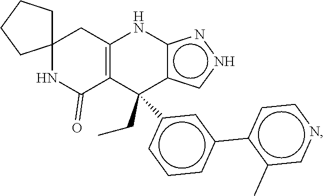 | 948021 |
| 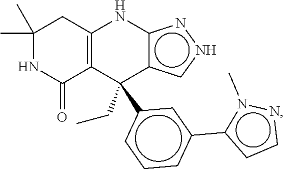 | 927390 |
| 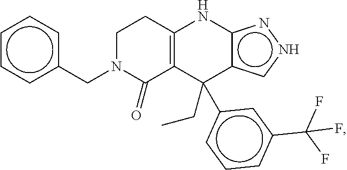 | 897749 |
| 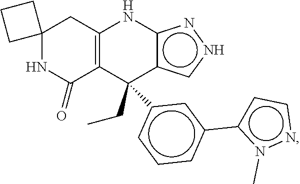 | 948522 |
| 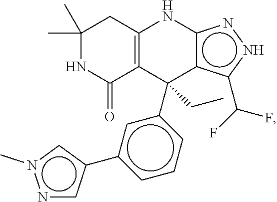 | 949911 |
| 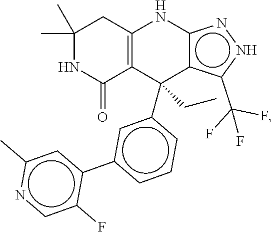 | 949644 |
| 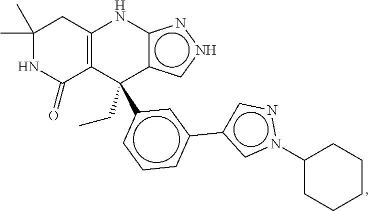 | 951707 |
| 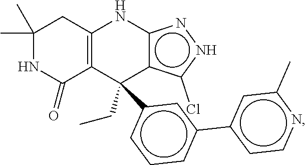 | 949421 |
| 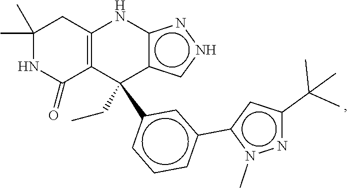 | 951843 |
| 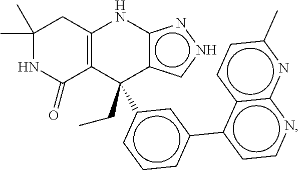 | 948151 |
| 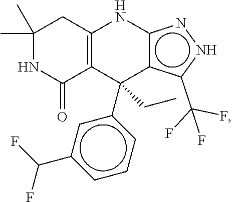 | 951946 |
| 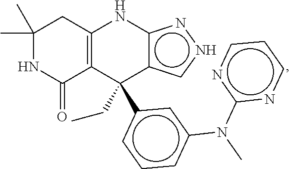 | 943965 |
| 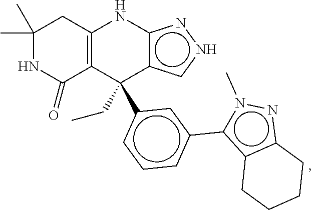 | 948652 |
| 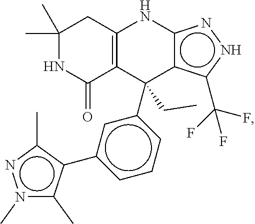 | 948417 |
| 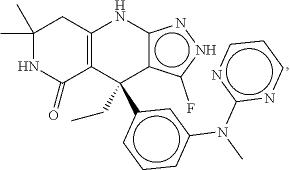 | 949647 |
| 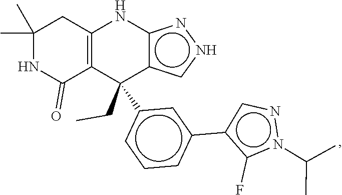 | 949267 |
| 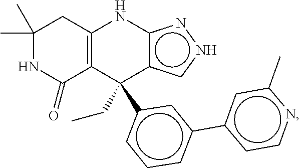 | 927326 |
| 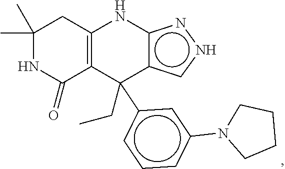 | 945723 |
| 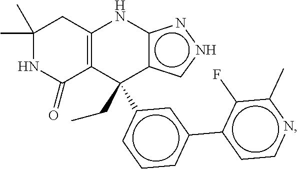 | 948311 |
| 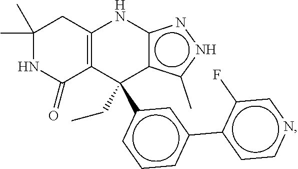 | 947805 |
| 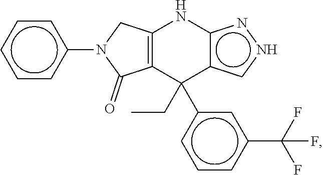 | 918313 |
| 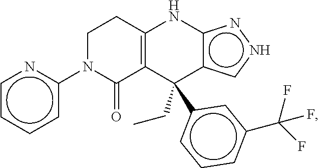 | 949311 |
| 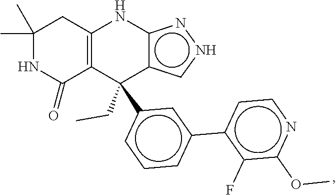 | 948531 |
| 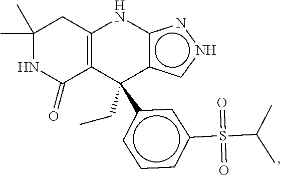 | 947823 |
| 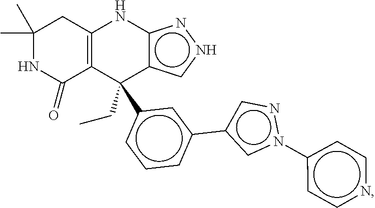 | 952912 |
| 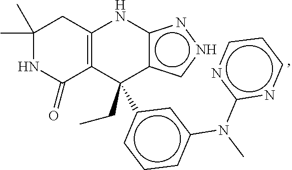 | 947825 |
| 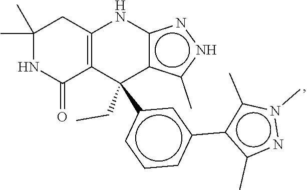 | 947573 |
| 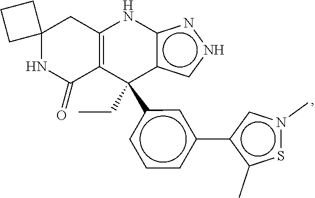 | 950056 |
| 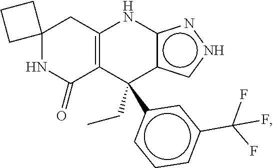 | 914344 |
| 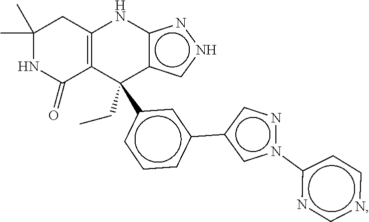 | 952016 |
| 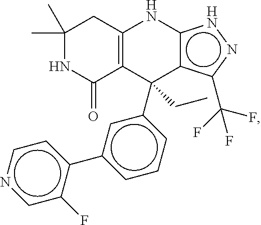 | 948770 |
| 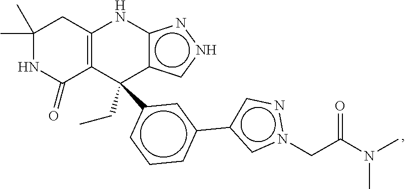 | 952208 |
| 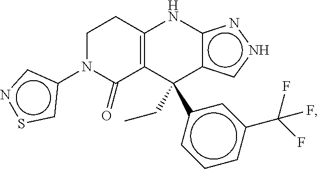 | 951742 |
| 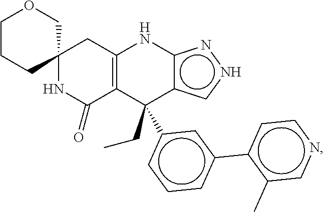 | 947912 |
| 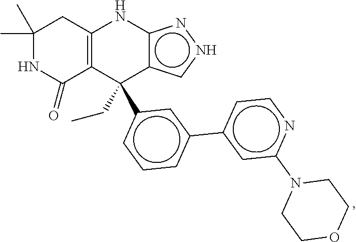 | 948825 |
| 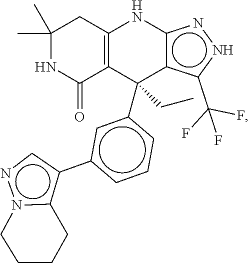 | 948772 |
| 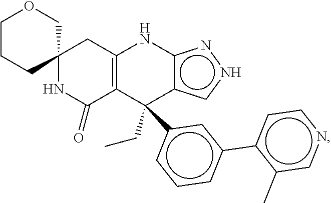 | 947909 |
| 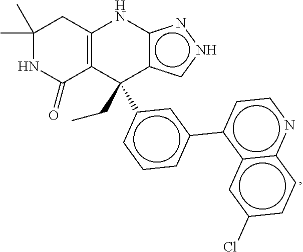 | 948530 |
| 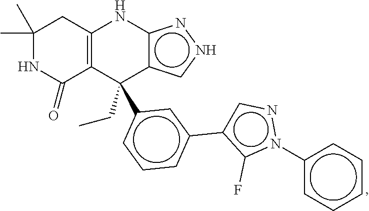 | 950821 |
| 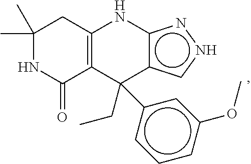 | 948296 |
| 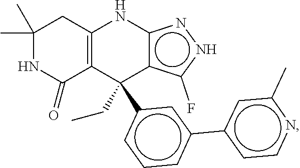 | 949001 |
| 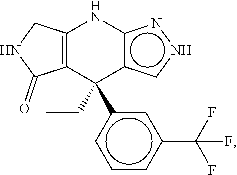 | 917158 |
| 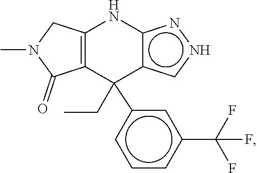 | 917294 |
| 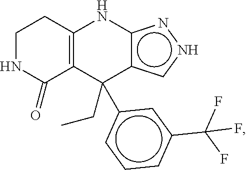 | 870263 |
| 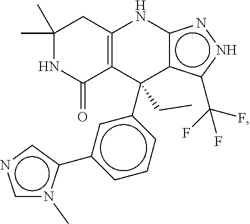 | 949003 |
| 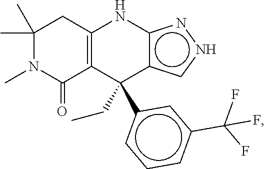 | 826791 |
| 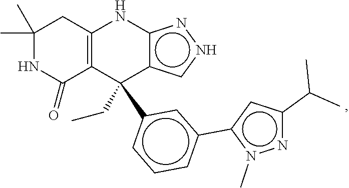 | 951842 |
| 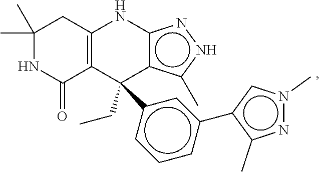 | 945996 |
| 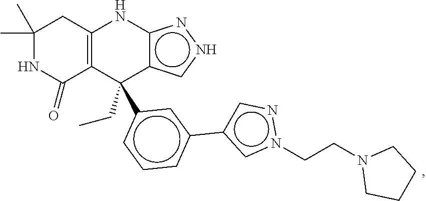 | 952022 |
| 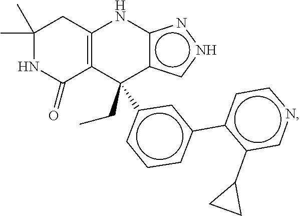 | 948642 |
| 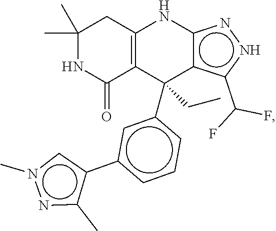 | 950490 |
| 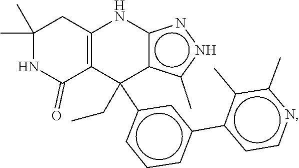 | 947399 |
| 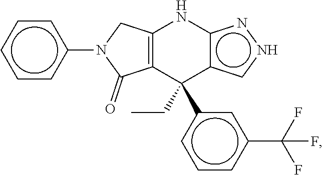 | 945377 |
| 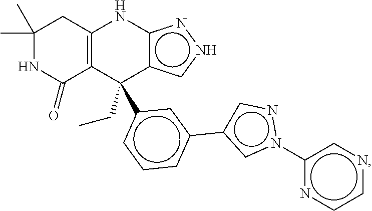 | 952207 |
| 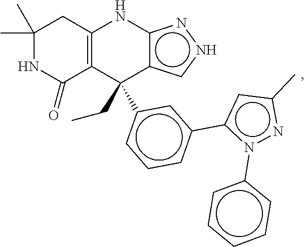 | 951845 |
| 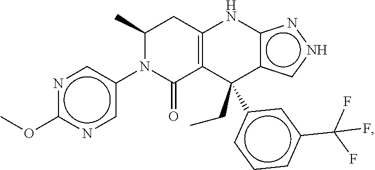 | 949354 |
| 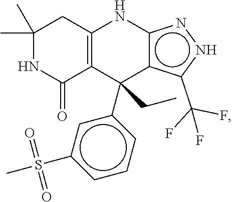 | 948422 |
| 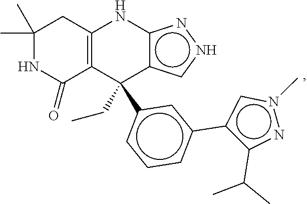 | 949111 |
| 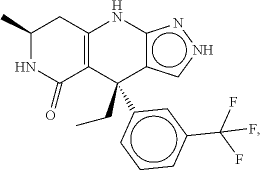 | 912097 |
| 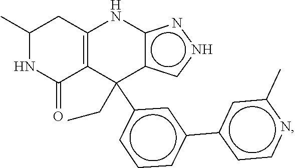 | 913357 |
| 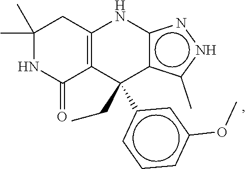 | 949337 |
| 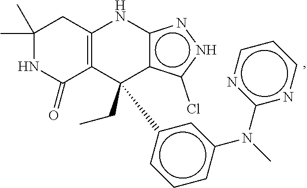 | 951618 |
| 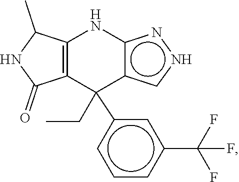 | 917295 |
| 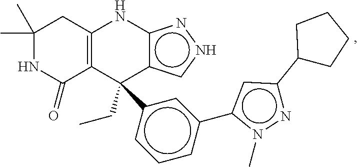 | 952023 |
| 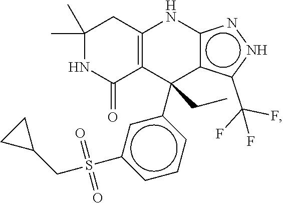 | 949186 |
| 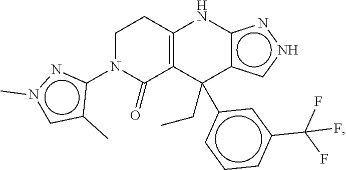 | 950486 |
| 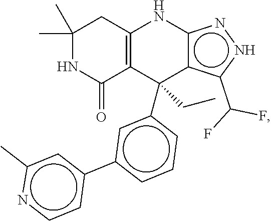 | 950162 |
| 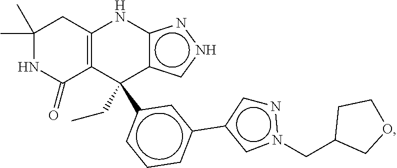 | 951836 |
| 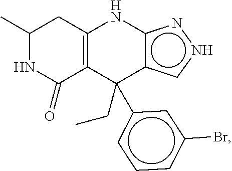 | 913225 |
| 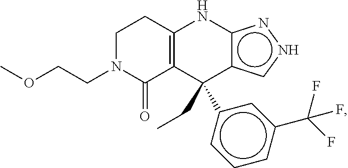 | 949599 |
| 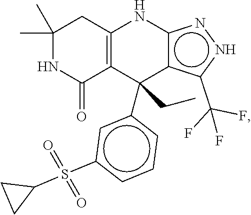 | 948776 |
| 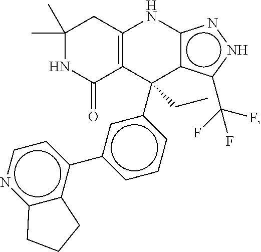 | 948769 |
| 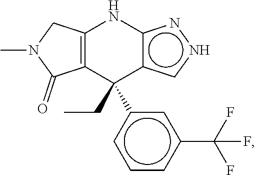 | 945367 |
| 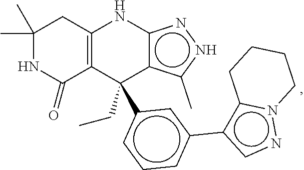 | 947809 |
| 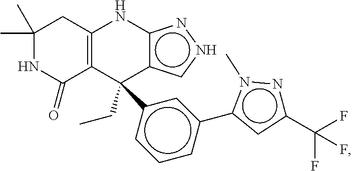 | 952018 |
| 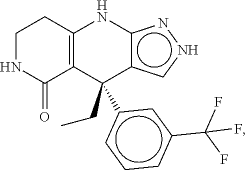 | 912622 |
| 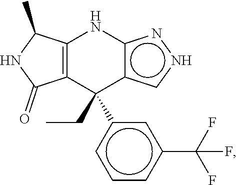 | 945379 |
| 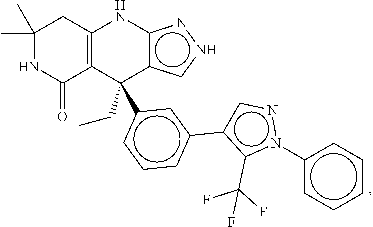 | 951837 |
| 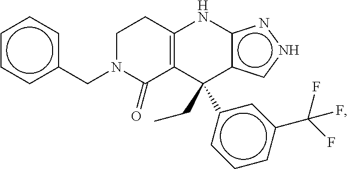 | 912005 |
| 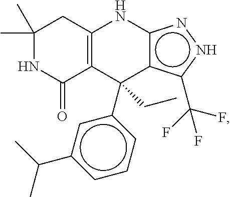 | 950493 |
| 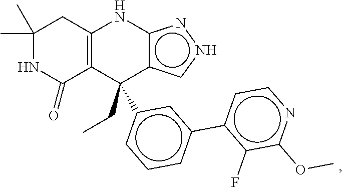 | 948520 |
| 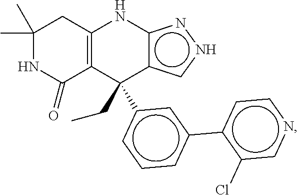 | 948020 |
| 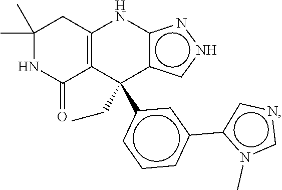 | 947820 |
| 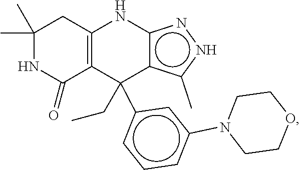 | 947564 |
| 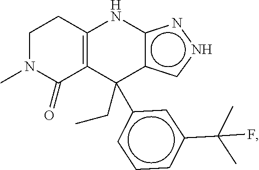 | 913178 |
| 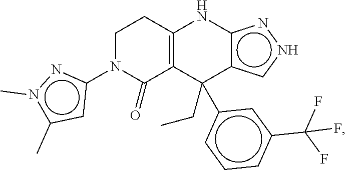 | 950608 |
| 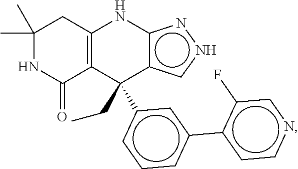 | 947874 |
| 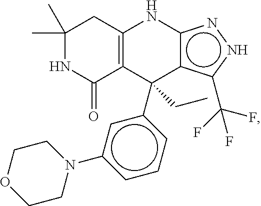 | 949000 |
| 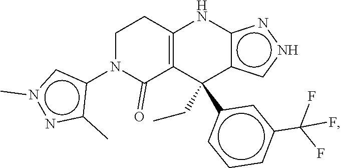 | 949642 |
| 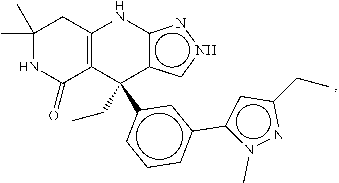 | 952561 |
| 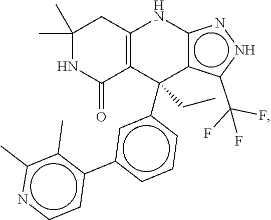 | 948181 |
| 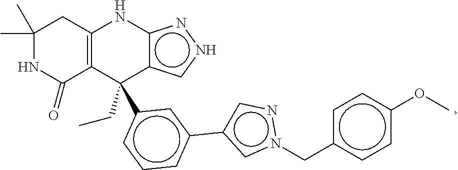 | 951838 |
| 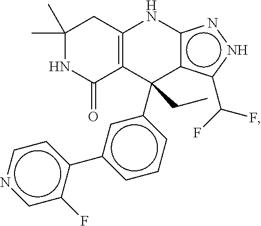 | 951599 |
| 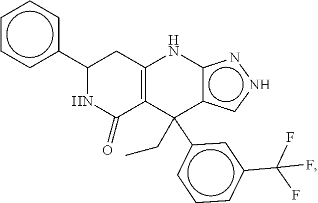 | 917284 |
| 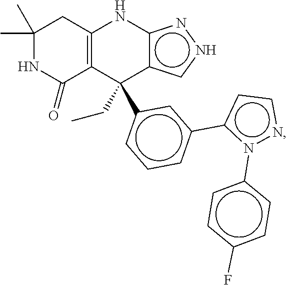 | 950822 |
| 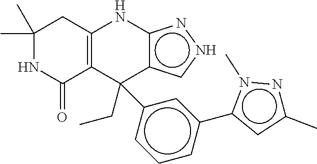 | 943371 |
| 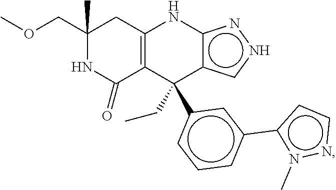 | 947905 |
| 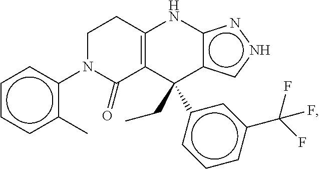 | 949128 |
| 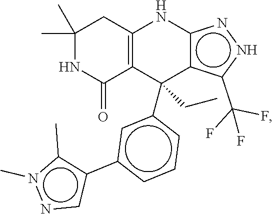 | 947807 |
| 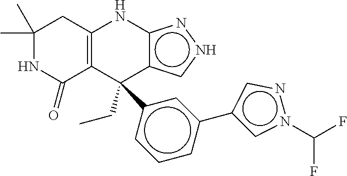 | 950254 |
| 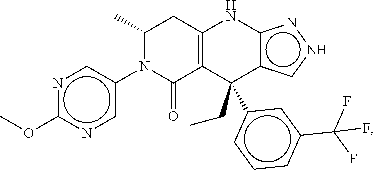 | 949353 |
| 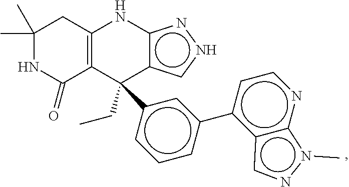 | 952913 |
| 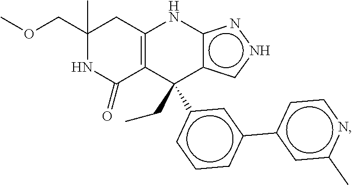 | 948521 |
| 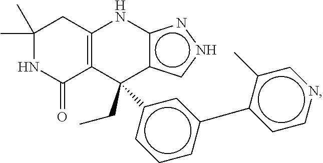 | 947576 |
| 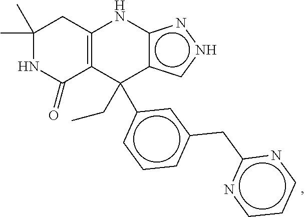 | 950151 |
| 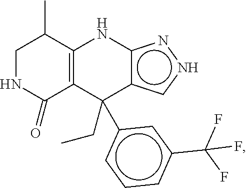 | 919318 |
| 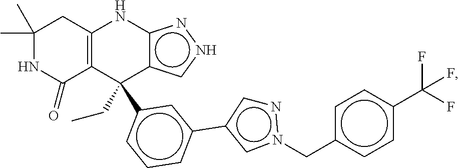 | 951849 |
| 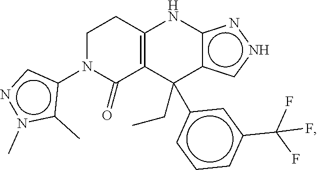 | 950612 |
| 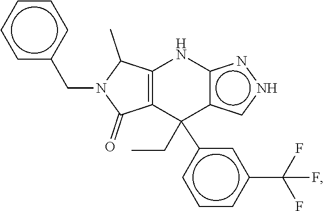 | 917162 |
| 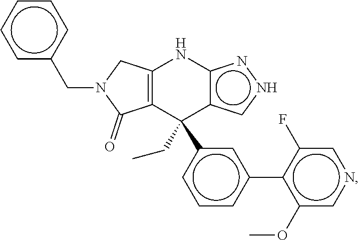 | 948550 |
| 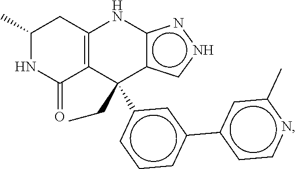 | 926212 |
| 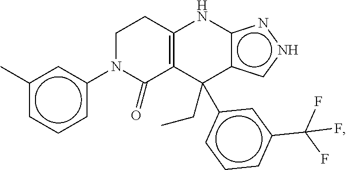 | 948052 |
| 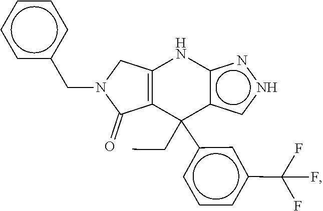 | 917159 |
| 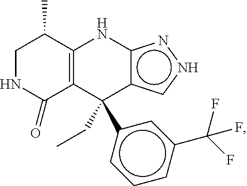 | 945382 |
| 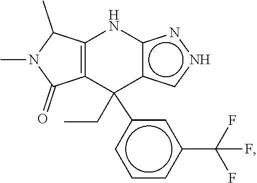 | 911957 |
| 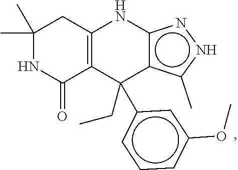 | 948763 |
| 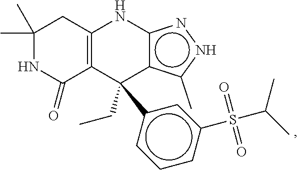 | 947870 |
| 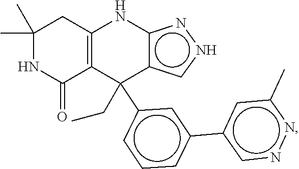 | 943496 |
| 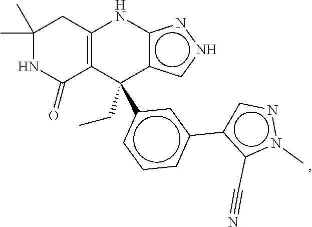 | 948827 |
| 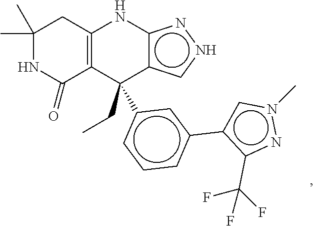 | 949266 |
| 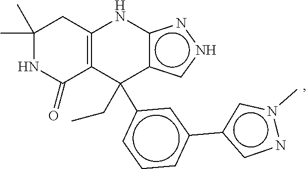 | 943912 |
| 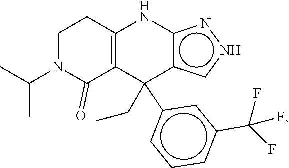 | 825755 |
| 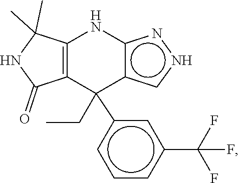 | 912104 |
| 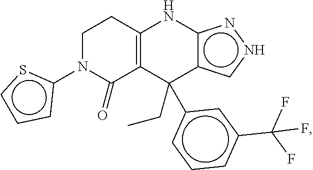 | 950614 |
| 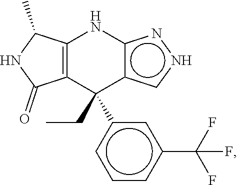 | 945378 |
| 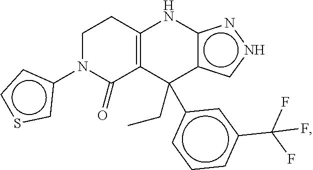 | 950776 |
| 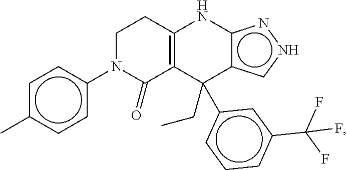 | 948241 |
| 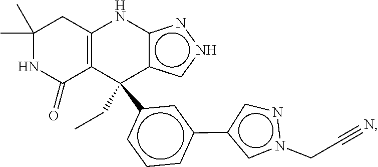 | 952210 |
| 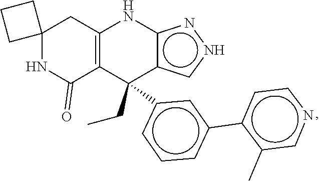 | 948523 |
| 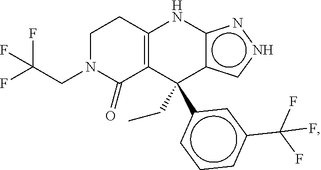 | 912206 |
| 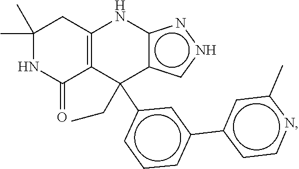 | 914930 |
| 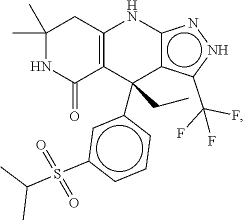 | 948423 |
| 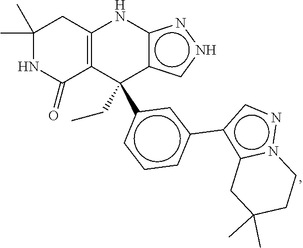 | 950564 |
| 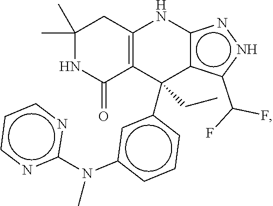 | 951736 |
| 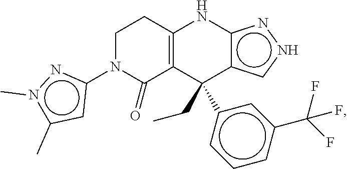 | 951939 |
| 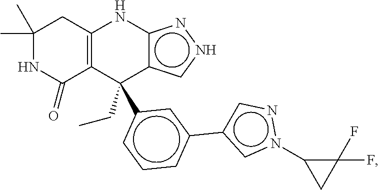 | 950060 |
| 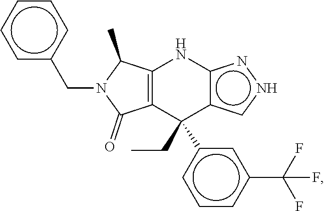 | 930331 |
| 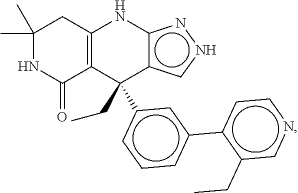 | 948639 |
| 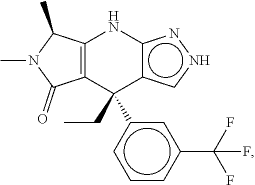 | 913473 |
| 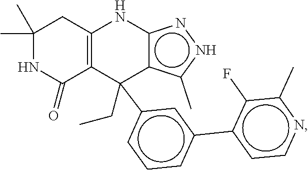 | 947563 |
| 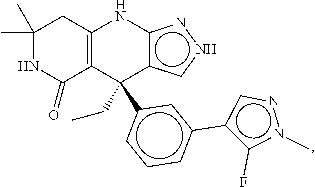 | 950562 |
| 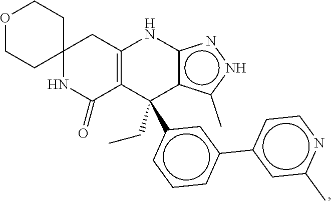 | 948513 |
| 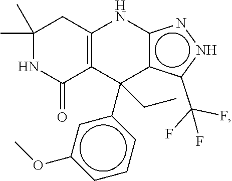 | 949748 |
| 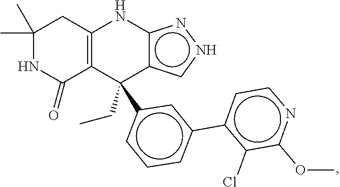 | 952754 |
| 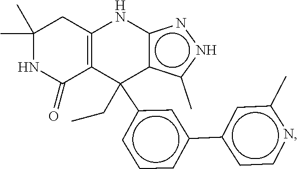 | 947798 |
| 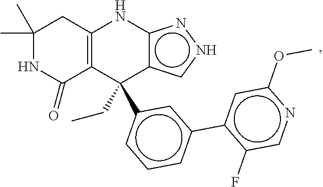 | 949109 |
| 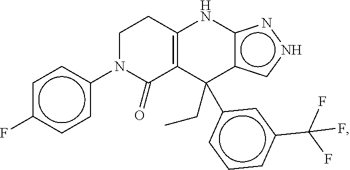 | 947748 |
| 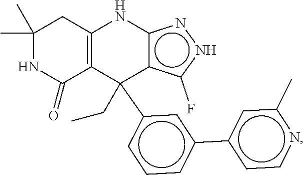 | 948299 |
| 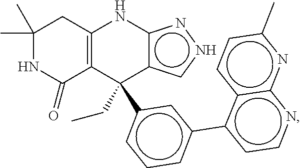 | 948143 |
| 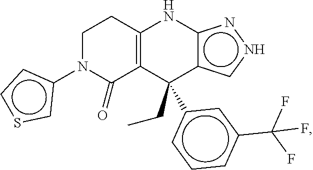 | 952630 |
| 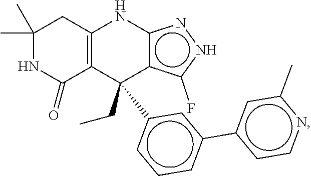 | 949012 |
| 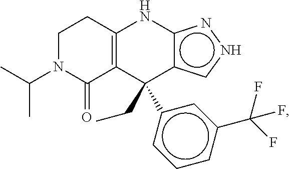 | 857336 |
| 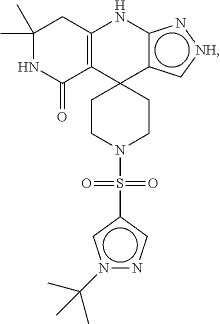 | 947835 |
| 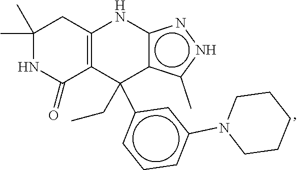 | 947674 |
| 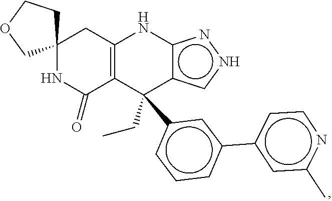 | 948149 |
| 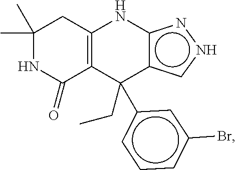 | 913439 |
| 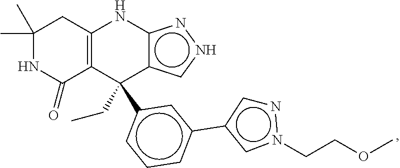 | 949105 |
| 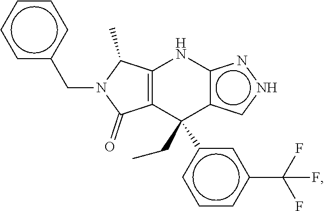 | 930330 |
| 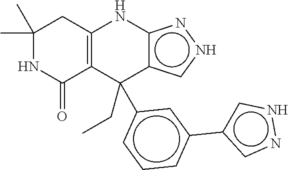 | 944022 |
| 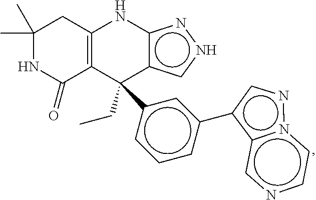 | 952565 |
| 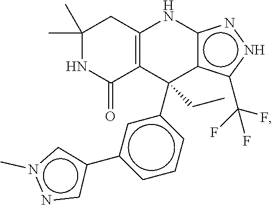 | 947871 |
| 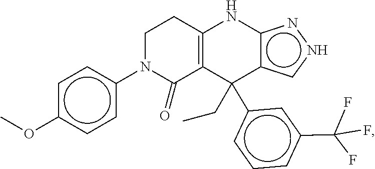 | 947332 |
| 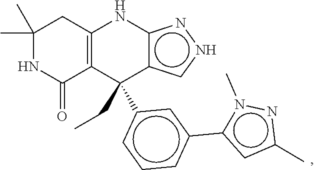 | 943966 |
| 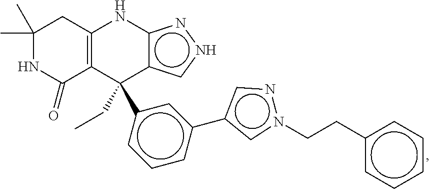 | 952017 |
| 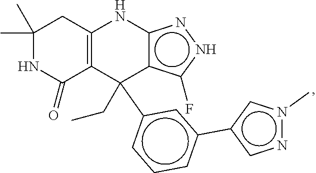 | 948300 |
| 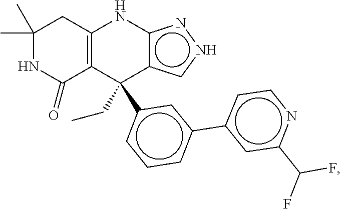 | 950561 |
| 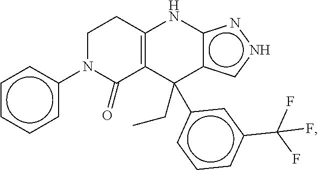 | 917211 |
| 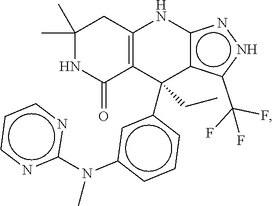 | 949180 |
| 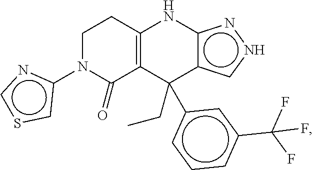 | 950777 |
| 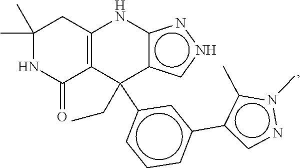 | 944021 |
| 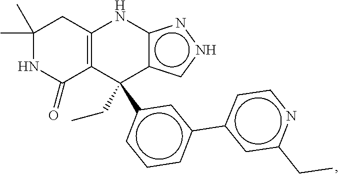 | 948370 |
| 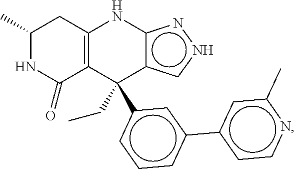 | 926300 |
| 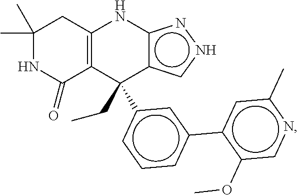 | 949522 |
| 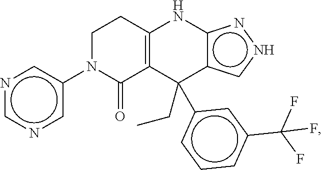 | 948238 |
| 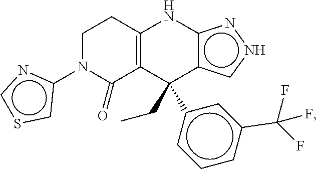 | 952629 |
| 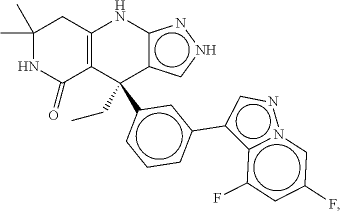 | 950565 |
| 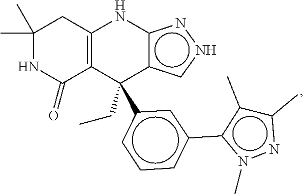 | 950062 |
| 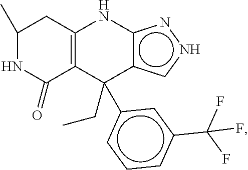 | 911956 |
| 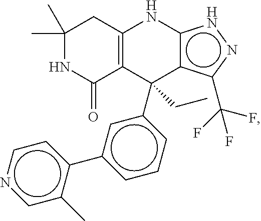 | 947812 |
| 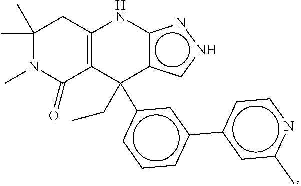 | 948302 |
| 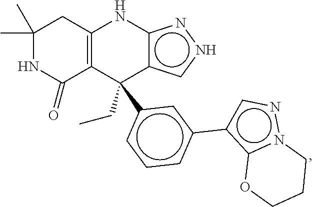 | 952019 |
| 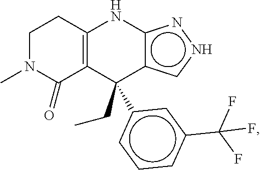 | 917282 |
| 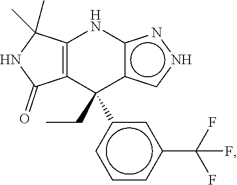 | 913451 |
| 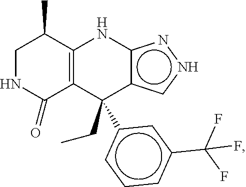 | 945383 |
| 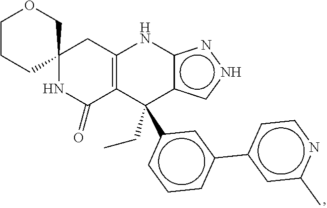 | 948152 |
| 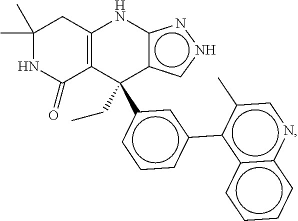 | 949398 |
| 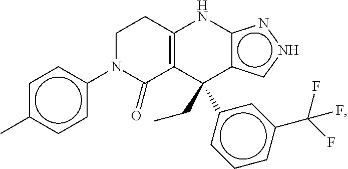 | 949118 |
| 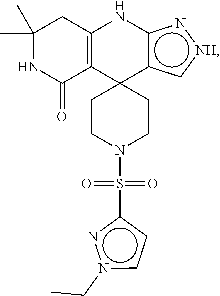 | 947836 |
| 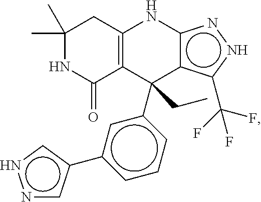 | 948312 |
| 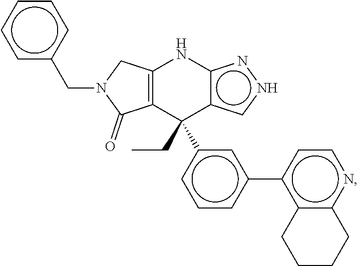 | 948551 |
| 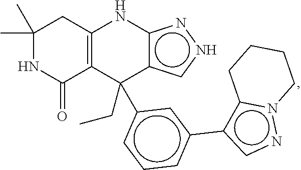 | 945603 |
| 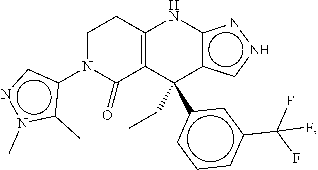 | 952257 |
| 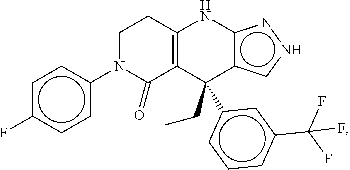 | 948224 |
| 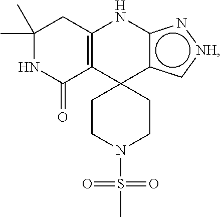 | 943959 |
| 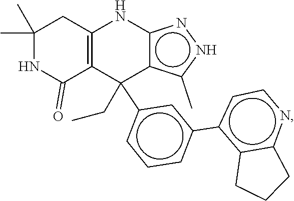 | 947675 |
| 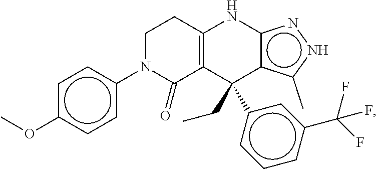 | 948226 |
| 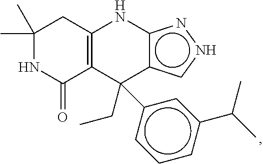 | 949326 |
| 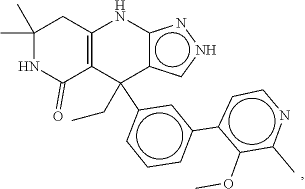 | 948794 |
| 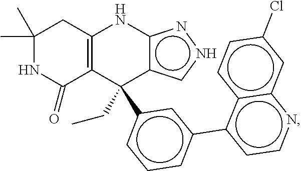 | 948368 |
| 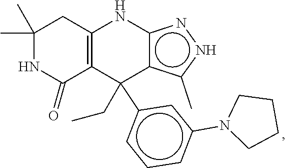 | 947673 |
| 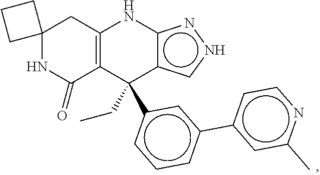 | 949520 |
| 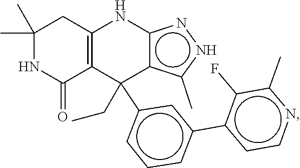 | 947402 |
| 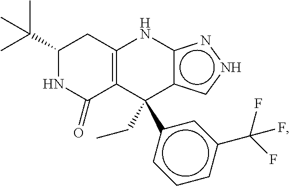 | 945922 |
| 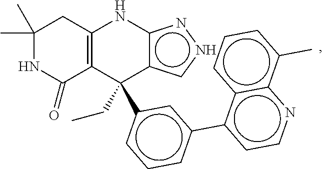 | 952909 |
| 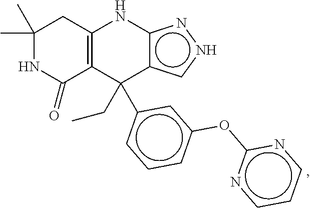 | 948764 |
| 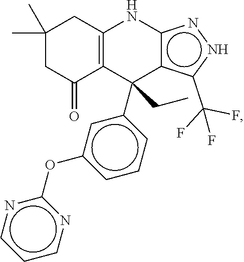 | 951604 |
| 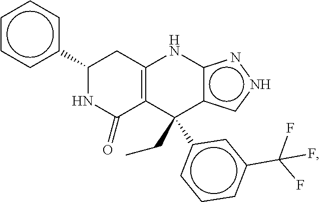 | 945361 |
| 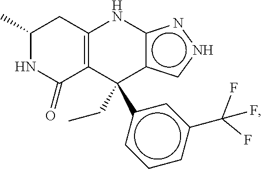 | 912098 |
| 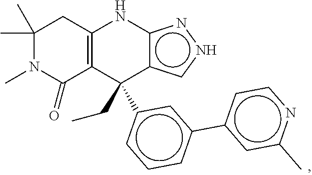 | 949004 |
| 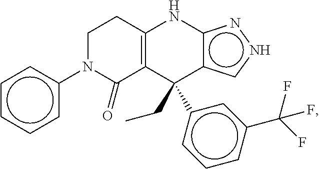 | 929474 |
| 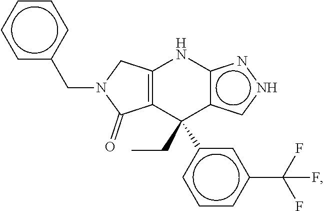 | 929388 |
| 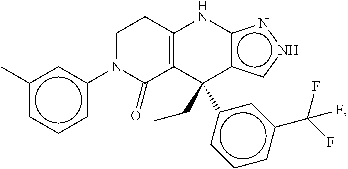 | 949040 |
| 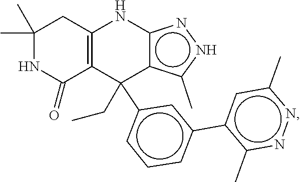 | 948766 |
| 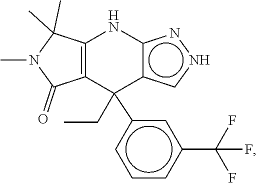 | 913198 |
| 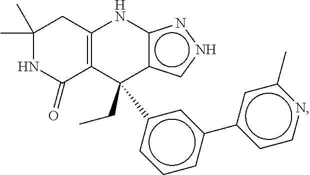 | 927325 |
| 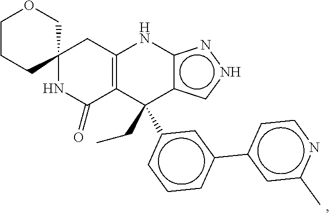 | 948144 |
| 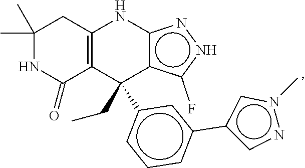 | 949011 |
| 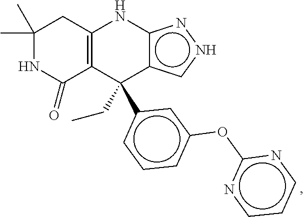 | 949330 |
| 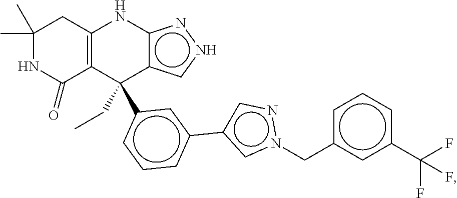 | 951848 |
| 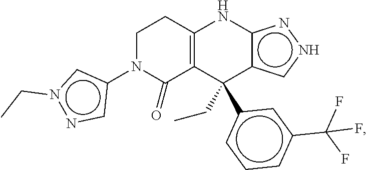 | 949867 |
| 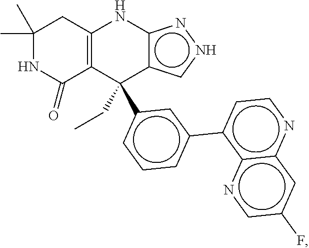 | 952758 |
| 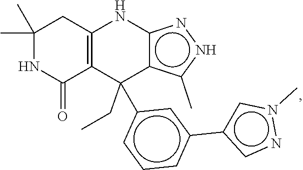 | 944027 |
| 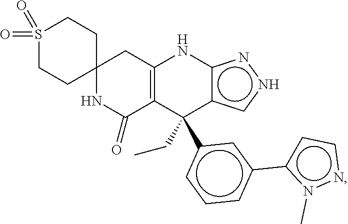 | 949394 |
| 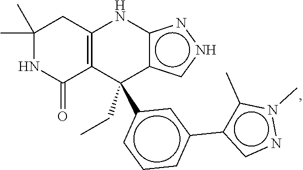 | 945608 |
| 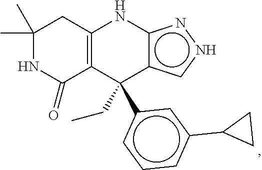 | 950828 |
| 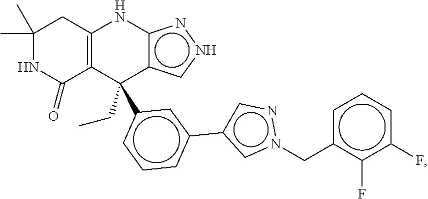 | 951840 |
| 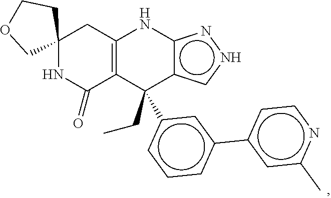 | 948140 |
| 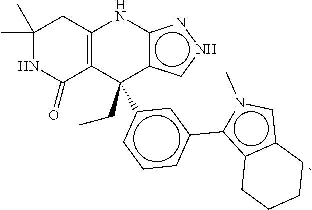 | 948655 |
| 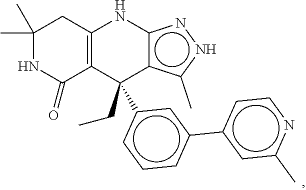 | 948420 |
| 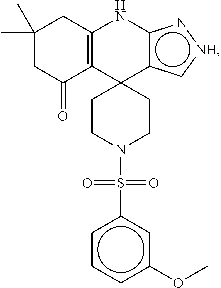 | 943962 |
| 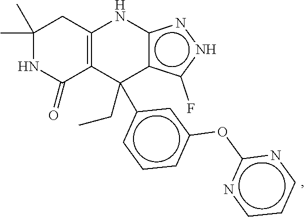 | 950615 |
| 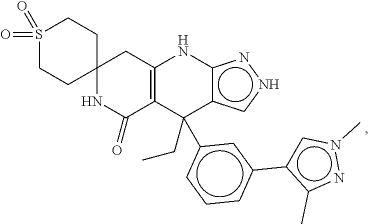 | 950813 |
| 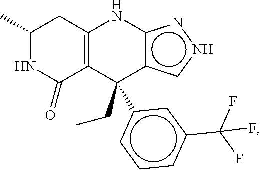 | 912096 |
| 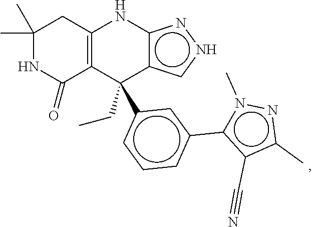 | 951844 |
| 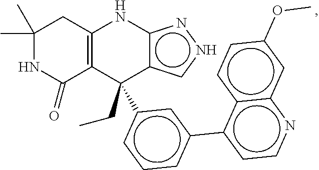 | 948661 |
| 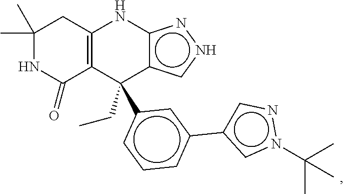 | 949112 |
| 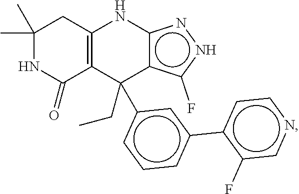 | 948999 |
| 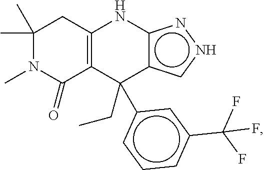 | 823130 |
| 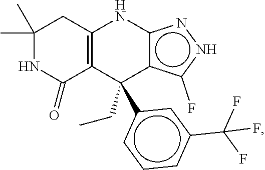 | 957518 |
| 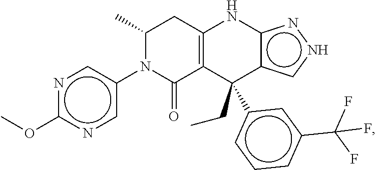 | 949351 |
| 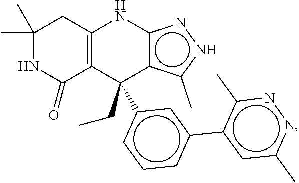 | 949333 |
| 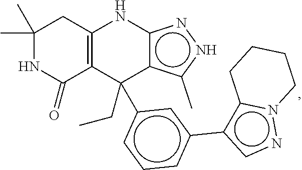 | 945993 |
| 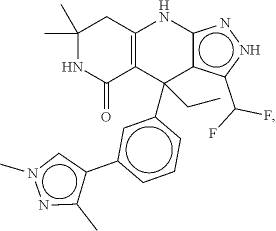 | 949170 |
| 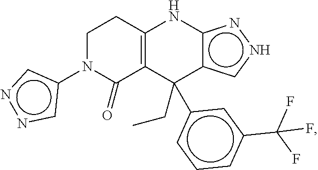 | 950479 |
| 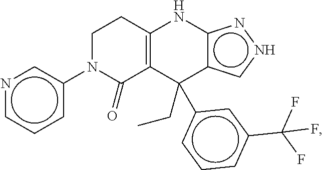 | 945637 |
| 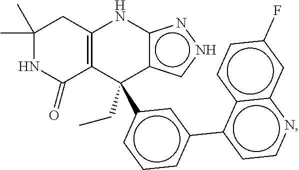 | 948528 |
| 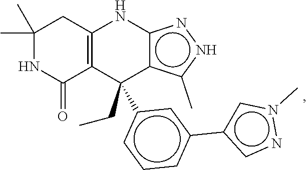 | 945614 |
| 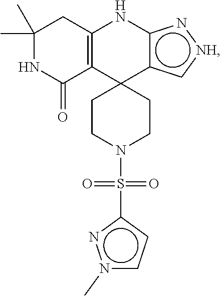 | 943961 |
| 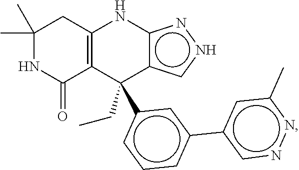 | 944074 |
| 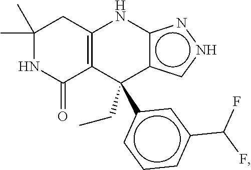 | 949006 |
| 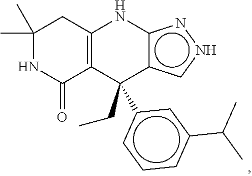 | 949761 |
| 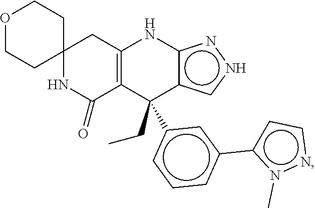 | 948516 |
| 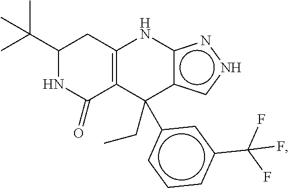 | 945635 |
| 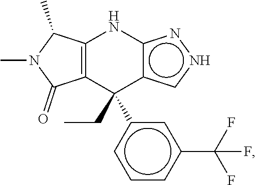 | 913454 |
| 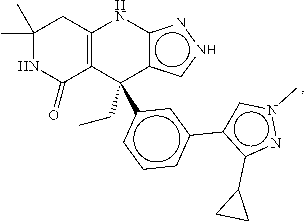 | 951706 |
| 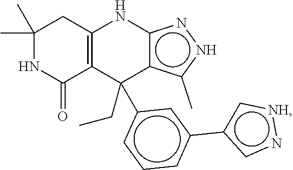 | 945459 |
| 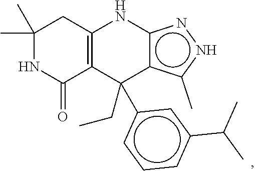 | 949173 |
| 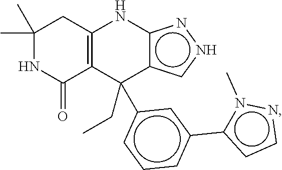 | 914971 |
| 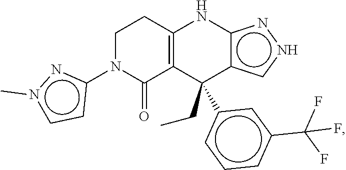 | 949835 |
| 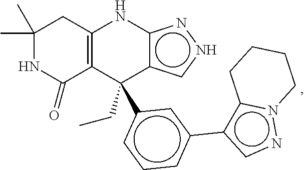 | 947410 |
| 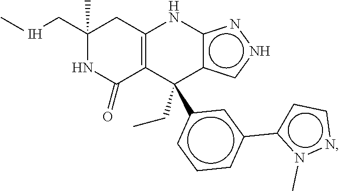 | 947906 |
| 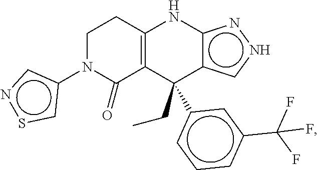 | 951733 |
| 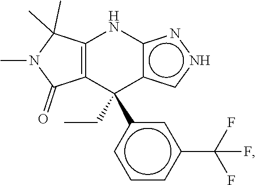 | 918252 |
| 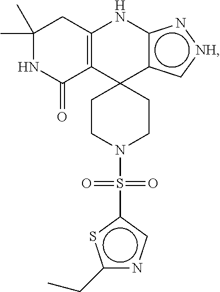 | 947837 |
| 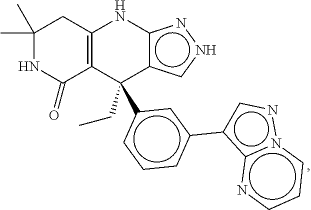 | 950830 |
| 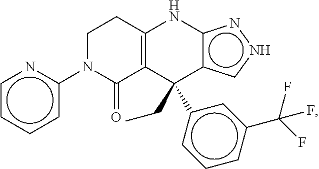 | 949310 |
| 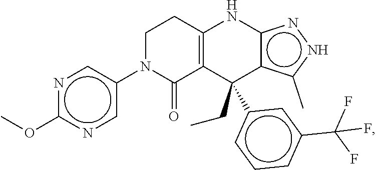 | 949126 |
| 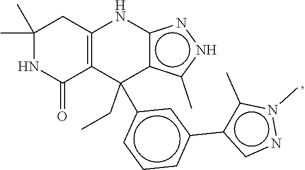 | 944028 |
| 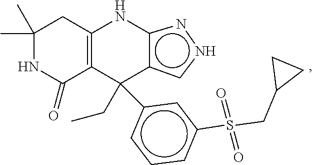 | 948177 |
| 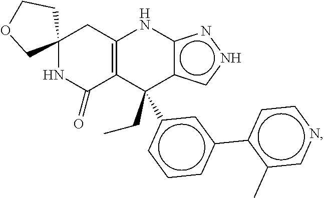 | 948640 |
| 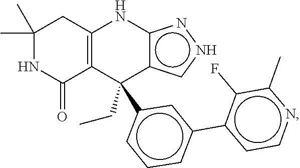 | 948306 |
| 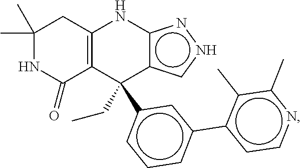 | 948184 |
| 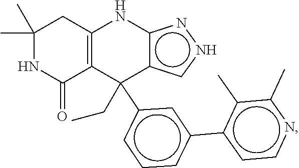 | 945994 |
| 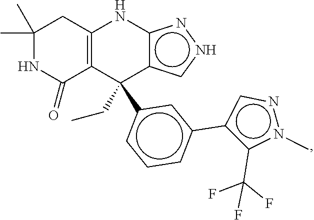 | 948815 |
| 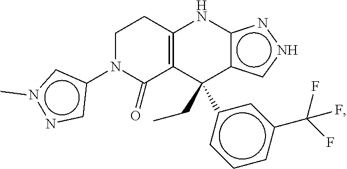 | 949270 |
| 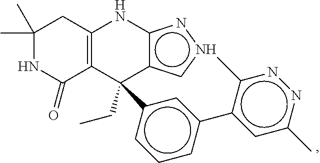 | 949331 |
| 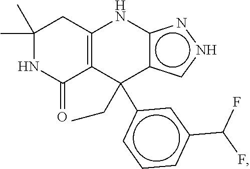 | 947864 |
| 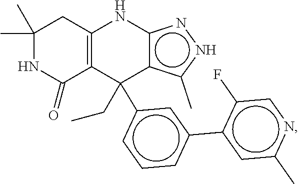 | 948761 |
| 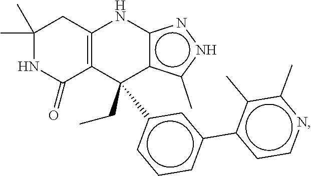 | 948187 |
| 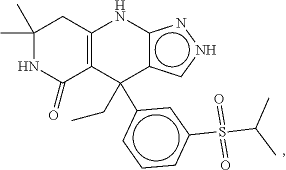 | 945464 |
| 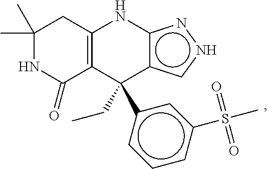 | 945604 |
| 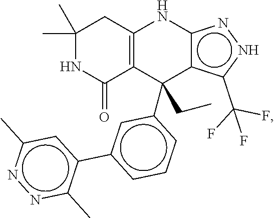 | 949910 |
| 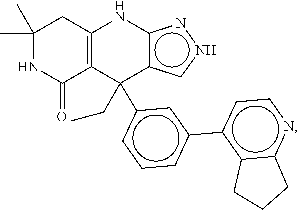 | 947676 |
| 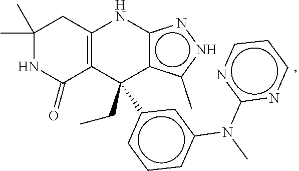 | 949182 |
| 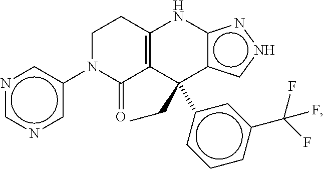 | 948379 |
| 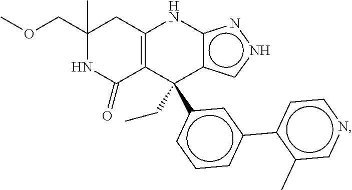 | 948147 |
| 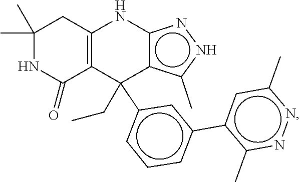 | 948765 |
| 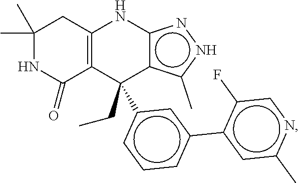 | 949338 |
| 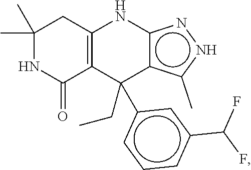 | 947865 |
| 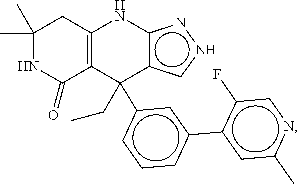 | 947401 |
| 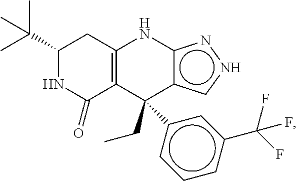 | 945920 |
| 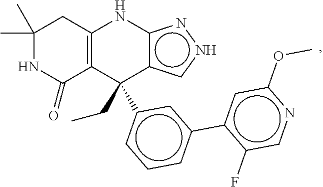 | 949108 |
| 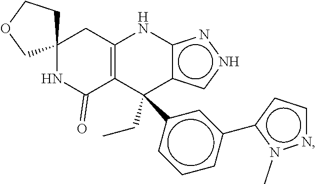 | 948015 |
| 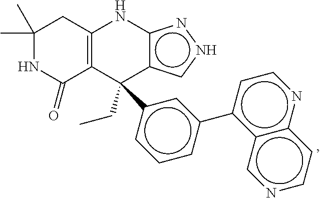 | 952910 |
| 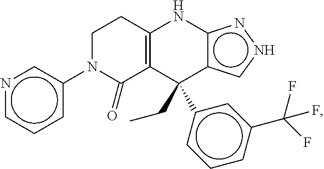 | 947432 |
| 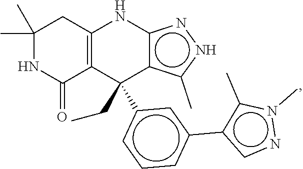 | 945731 |
| 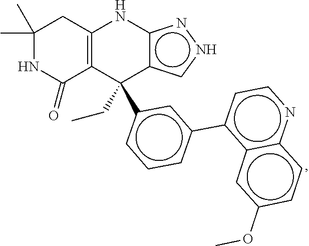 | 948527 |
| 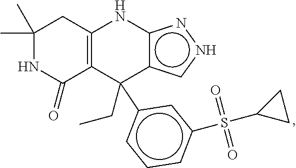 | 945465 |
| 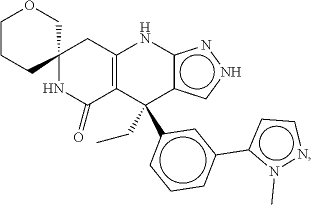 | 949115 |
| 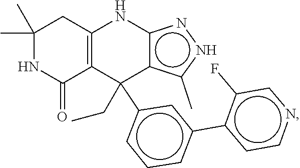 | 945722 |
| 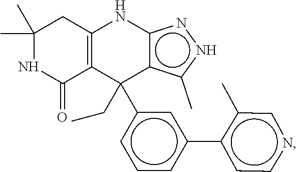 | 945599 |
| 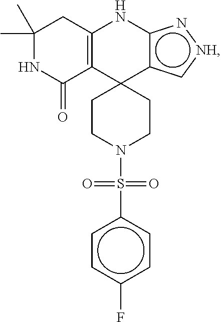 | 943963 |
| 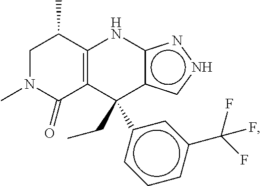 | 883979 |
| 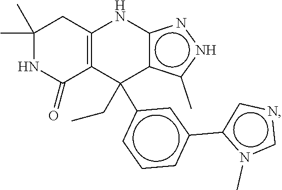 | 947565 |
| 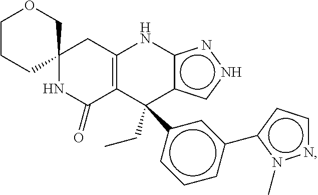 | 948529 |
| 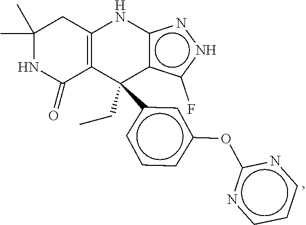 | 951949 |
| 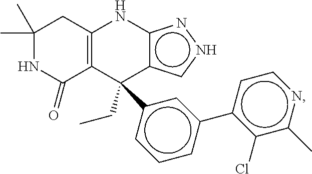 | 948361 |
| 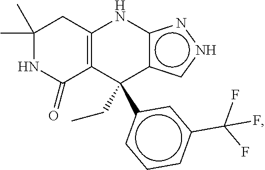 | 913006 |
| 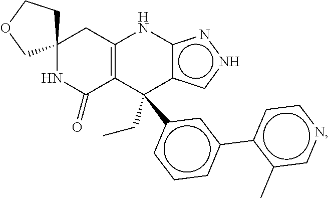 | 948644 |
| 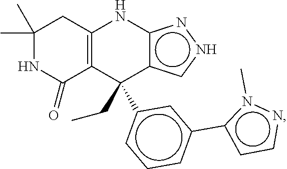 | 927389 |
| 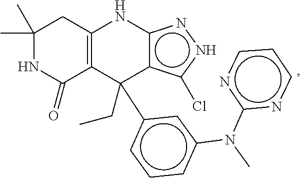 | 949417 |
| 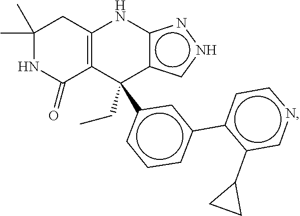 | 950059 |
| 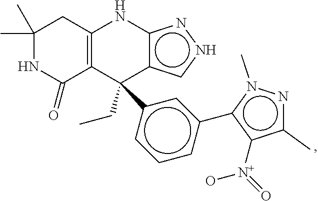 | 952209 |
| 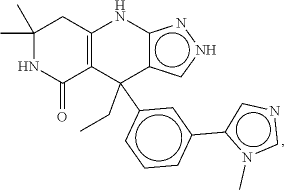 | 945597 |
| 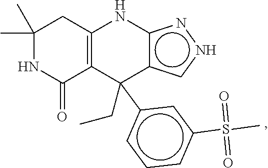 | 944020 |
| 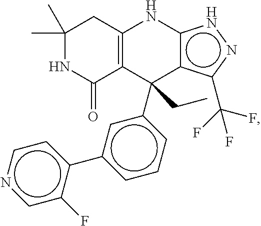 | 948777 |
| 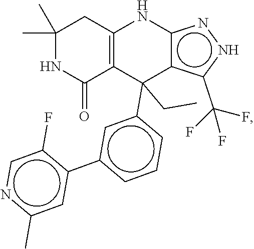 | 948762 |
| 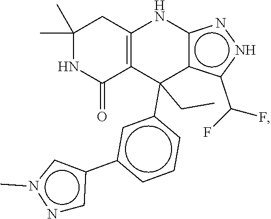 | 949171 |
| 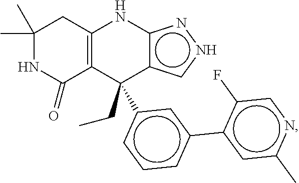 | 947804 |
| 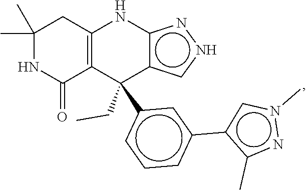 | 945470 |
| 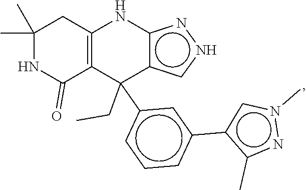 | 944030 |
| 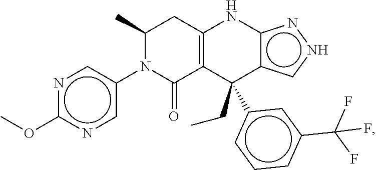 | 949352 |
| 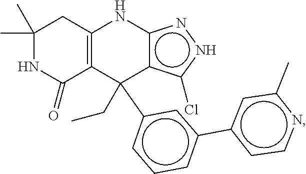 | 948998 |
| 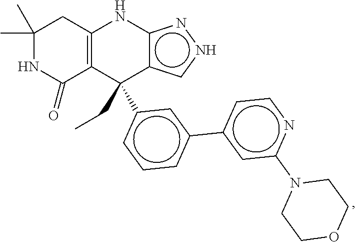 | 948653 |
| 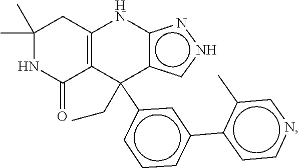 | 945461 |
| 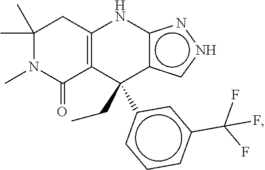 | 826790 |
| 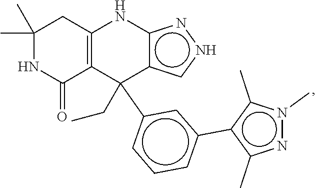 | 945462 |
| 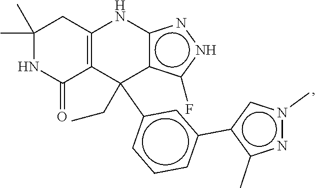 | 948301 |
| 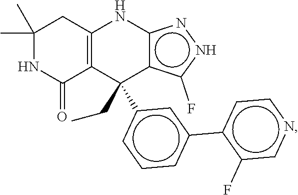 | 949651 |
| 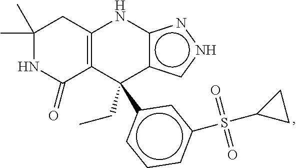 | 945999 |
| 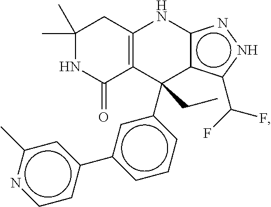 | 950163 |
| 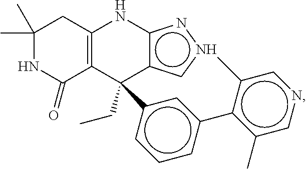 | 948812 |
| 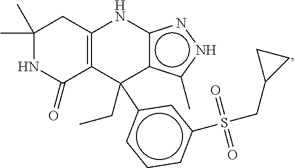 | 948178 |
| 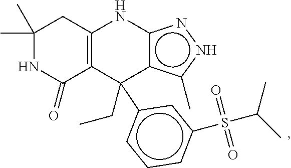 | 947396 |
| 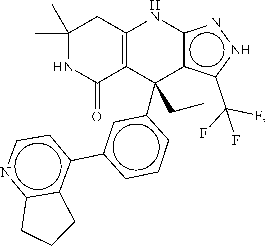 | 948778 |
| 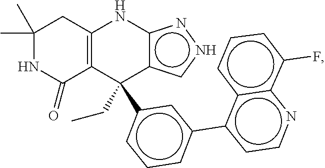 | 948828 |
| 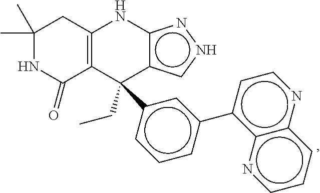 | 952560 |
| 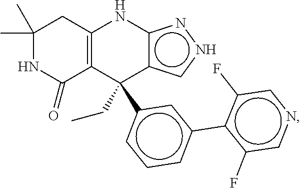 | 948525 |
| 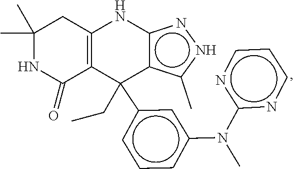 | 948175 |
| 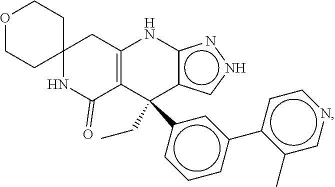 | 948359 |
| 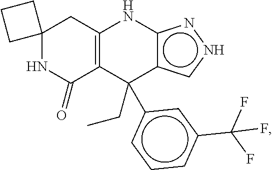 | 902202 |
| 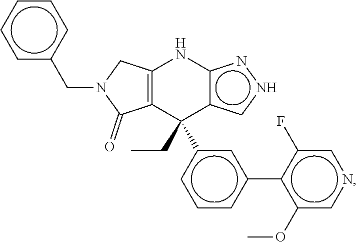 | 948549 |
| 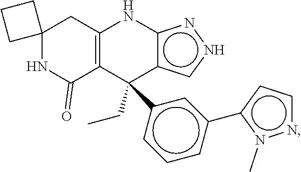 | 948358 |
| 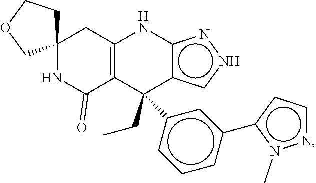 | 948017 |
| 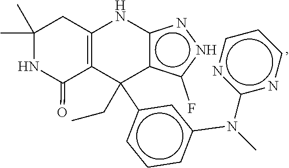 | 949172 |
| 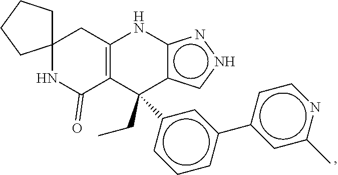 | 948660 |
| 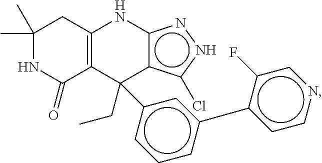 | 949324 |
| 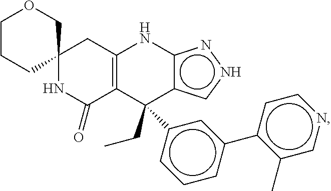 | 947911 |
| 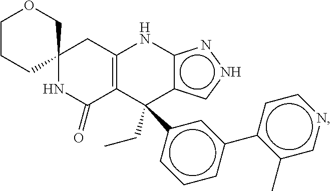 | 947910 |
| 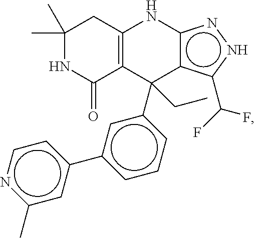 | 949169 |
| 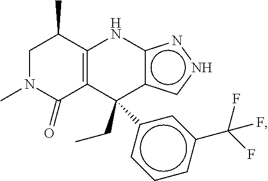 | 883980 |
| 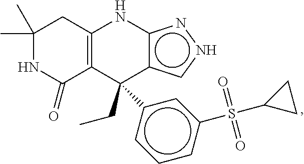 | 947803 |
| 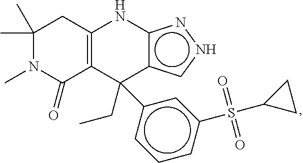 | 948303 |
| 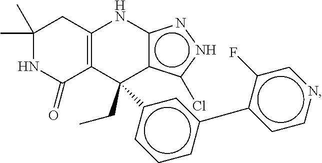 | 949757 |
| 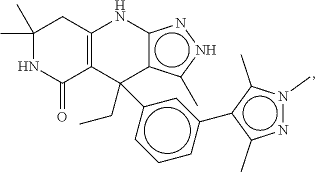 | 945598 |
| 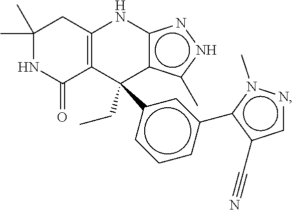 | 952410 |
| 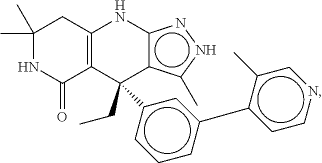 | 947414 |
| 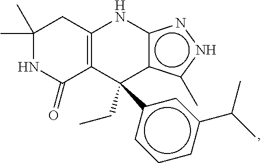 | 949649 |
| 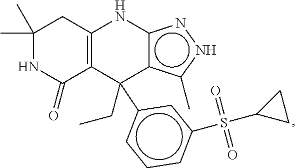 | 947562 |
| 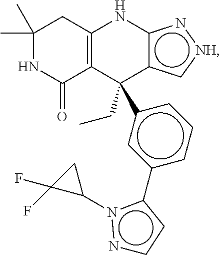 | 950824 |
| 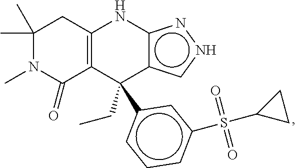 | 949177 |
| 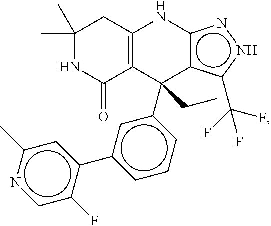 | 949650 |
| 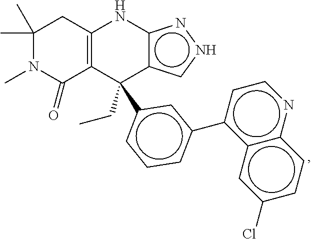 | 948517 |
| 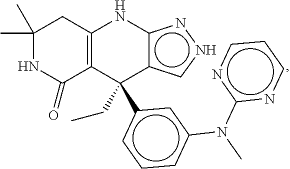 | 947826 |
| 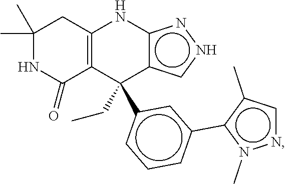 | 952021 |
| 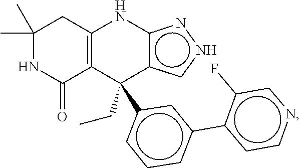 | 947869 |
| 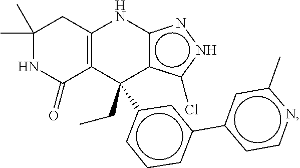 | 949422 |
| 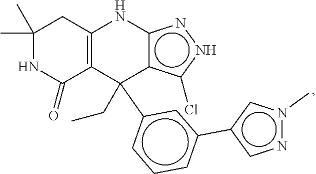 | 948767 |
| 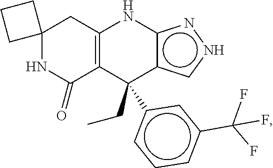 | 914308 |
| 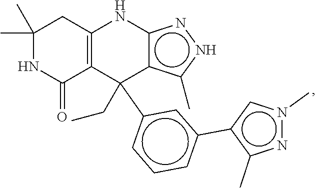 | 945460 |
| 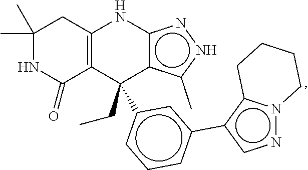 | 947817 |
| 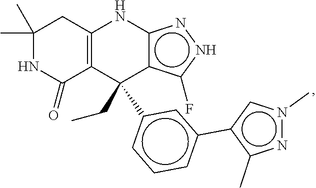 | 949185 |
| 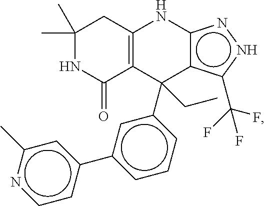 | 947858 |
| 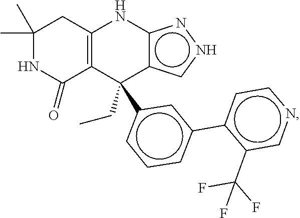 | 952025 |
| 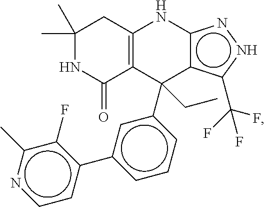 | 948297 |
| 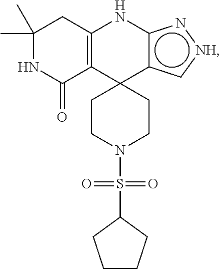 | 943960 |
| 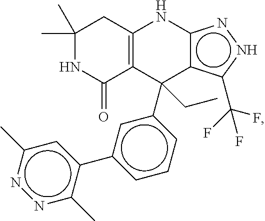 | 949174 |
| 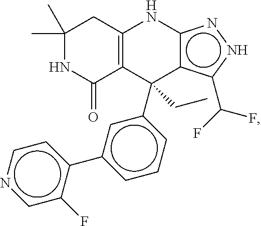 | 951612 |
| 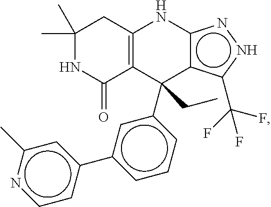 | 948774 |
| 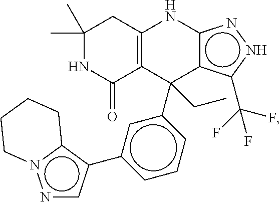 | 947801 |
| 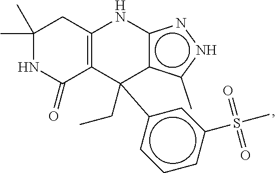 | 947395 |
| 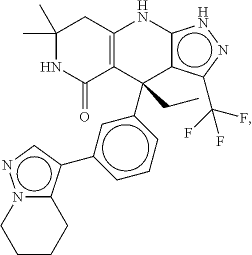 | 948775 |
| 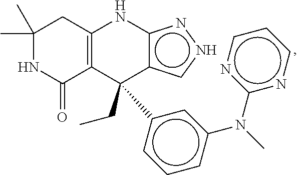 | 943964 |
| 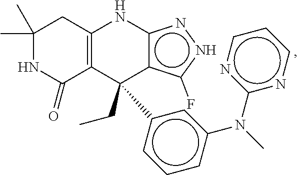 | 949648 |
| 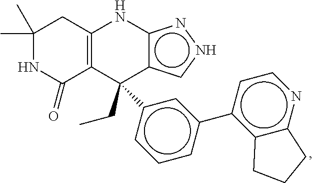 | 948313 |
| 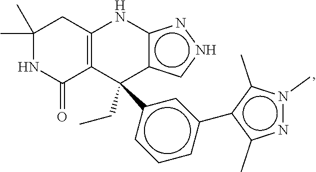 | 947408 |
| 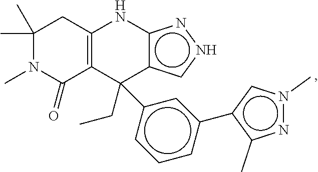 | 948304 |
| 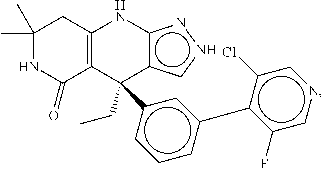 | 948371 |
| 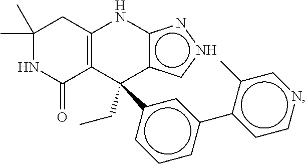 | 947574 |
| 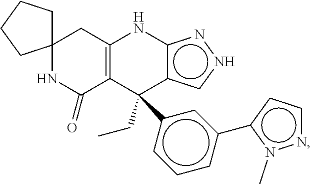 | 948018 |
| 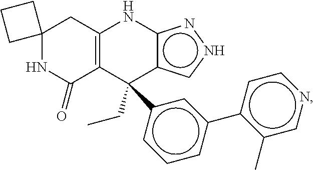 | 948512 |
| 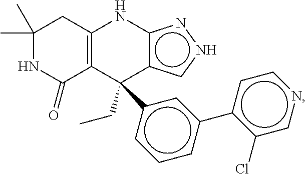 | 948142 |
| 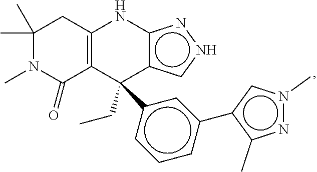 | 949005 |
| 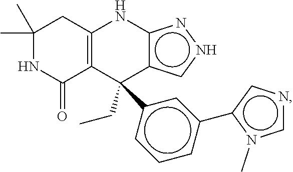 | 947806 |
| 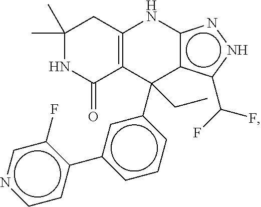 | 950320 |
| 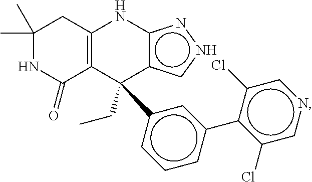 | 948515 |
| 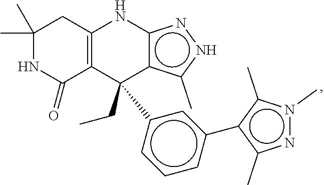 | 947575 |
| 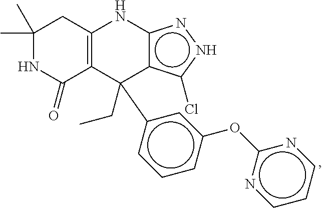 | 950318 |
| 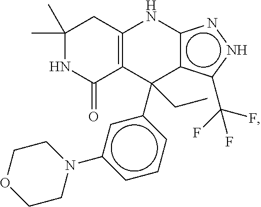 | 947863 |
| 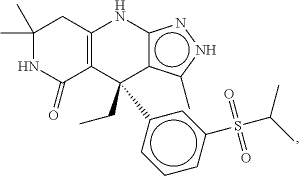 | 947876 |
| 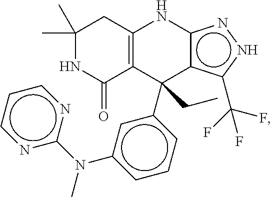 | 949181 |
| 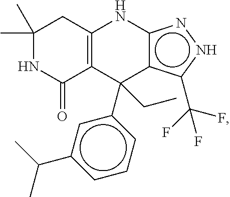 | 949747 |
| 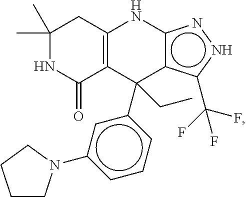 | 947860 |
| 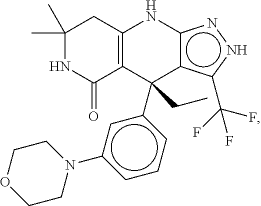 | 949013 |
| 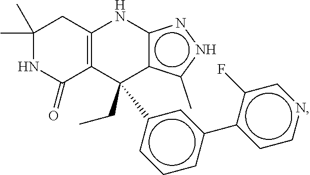 | 947821 |
| 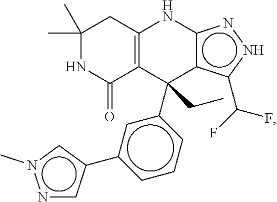 | 949919 |
| 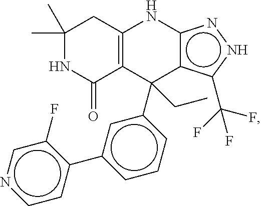 | 947866 |
| 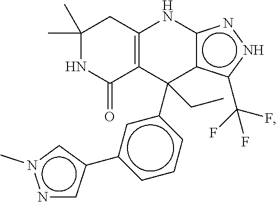 | 947405 |
| 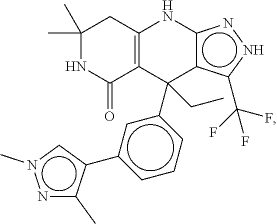 | 947567 |
| 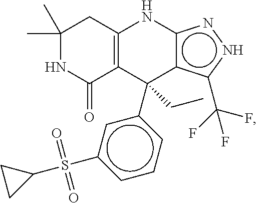 | 948771 |
| 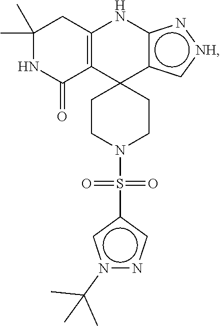 | 947835 |
| 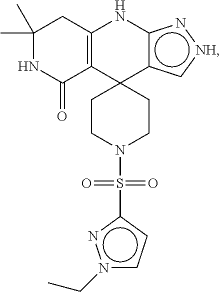 | 947836 |
| 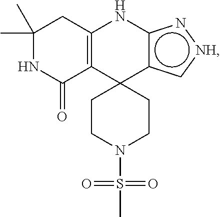 | 943959 |
| 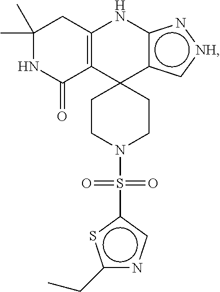 | 947837 |
| 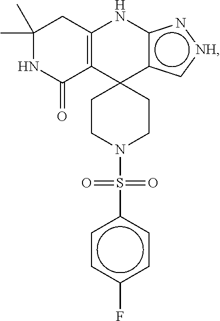 | 943963 |
| 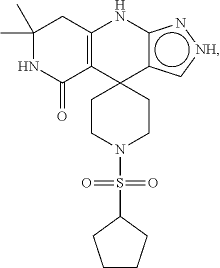 | 943960 |

30. The compound of claim 1, or a pharmaceutically acceptable salt, solvate, hydrate, polymorph, co-crystal, tautomer, stereoisomer, isotopically labeled compound, or prodrug thereof, wherein the compound is any one of the formulae shown in Table 1A:
TABLE 1A

| | |
|---|---|
| 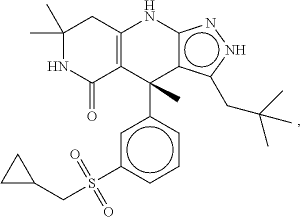 | 108005 |
| 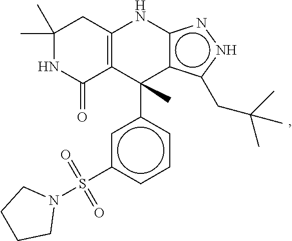 | 108791 |
| 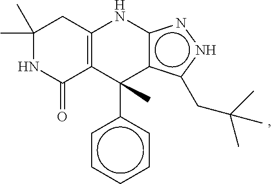 | 114531 |
| 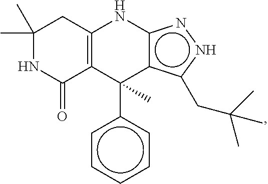 | 114532 |

or

| | |
|---|---|
| 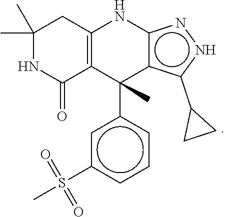 | 114679 |

31. The compound of claim 1, or a pharmaceutically acceptable salt, solvate, hydrate, polymorph, co-crystal, tautomer, stereoisomer, isotopically labeled compound, or prodrug thereof, wherein the compound is any one of the formulae shown in Table 2:
TABLE 2

| | |
|---|---|
| 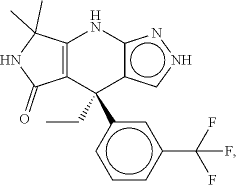 | 913452 |
| 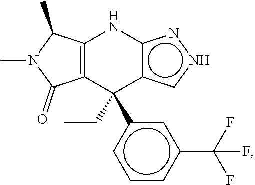 | 913495 |
| 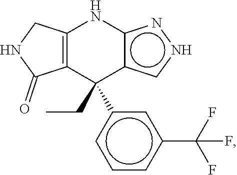 | 917157 |
| 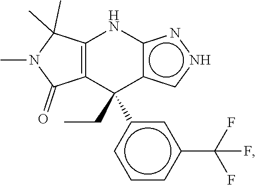 | 918251 |
| 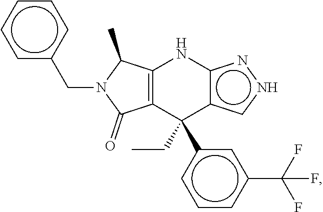 | 930333 |
| 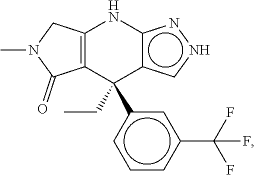 | 945368 |
| 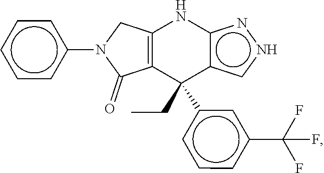 | 945376 |
| 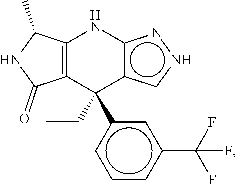 | 945380 |
| 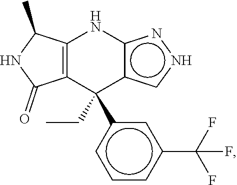 | 945381 |
| 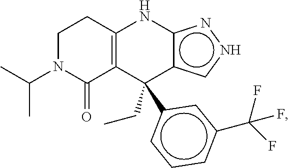 | 857362 |
| 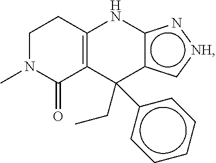 | 883867 |
| 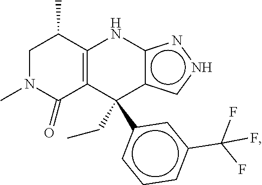 | 883981 |
| 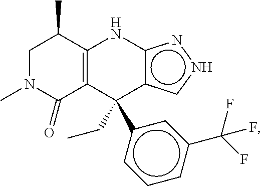 | 883982 |
| 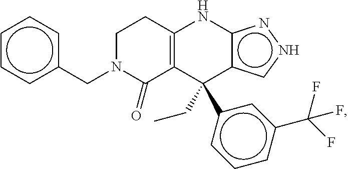 | 912006 |
| 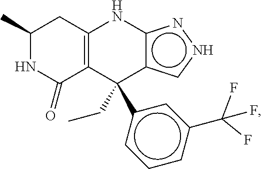 | 912099 |
| 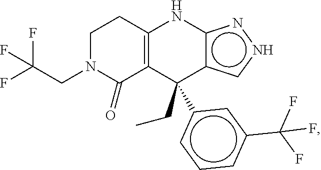 | 912205 |
| 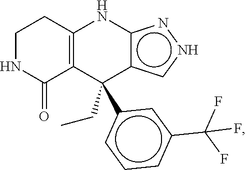 | 912668 |
| 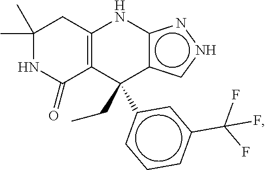 | 913004 |
| 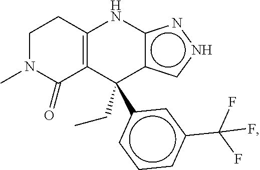 | 917283 |
| 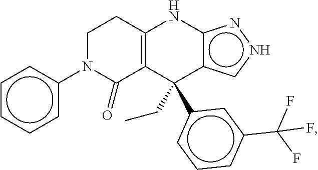 | 929475 |
| 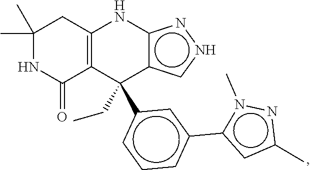 | 943967 |
| 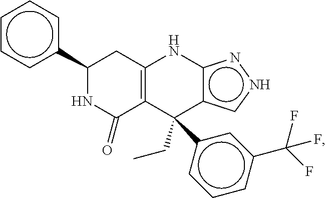 | 945364 |
| 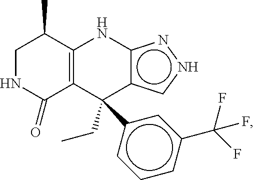 | 945385 |
| 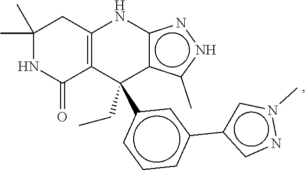 | 945611 |
| 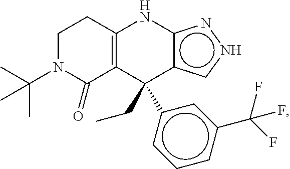 | 945633 |
| 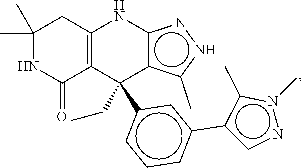 | 945727 |
| 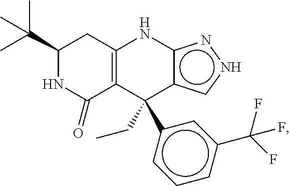 | 945923 |
| 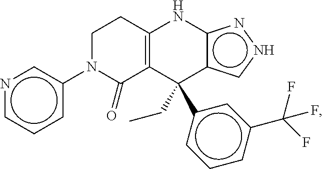 | 947433 |
| 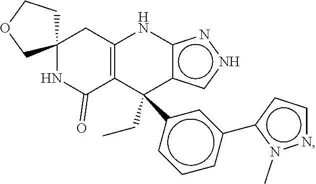 | 948016 |
| 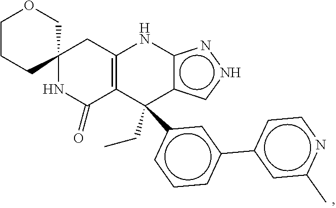 | 948153 |
| 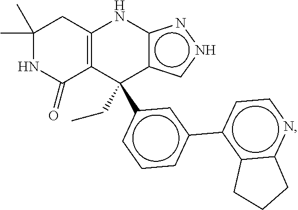 | 948308 |
| 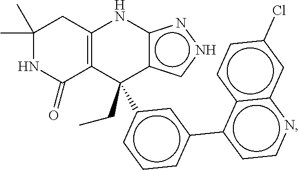 | 948362 |
| 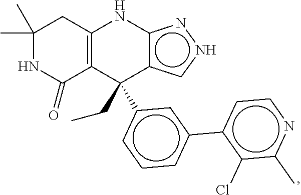 | 948369 |
| 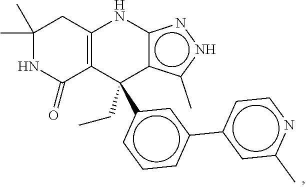 | 948414 |
| 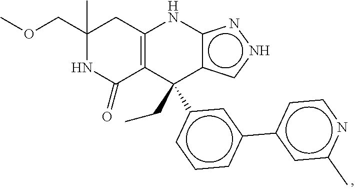 | 948493 |
| 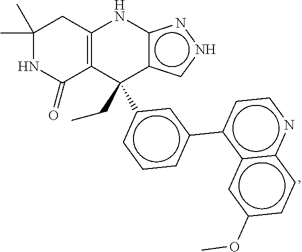 | 948518 |
| 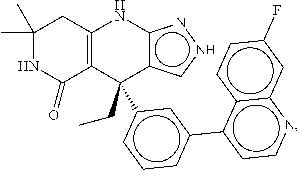 | 948519 |
| 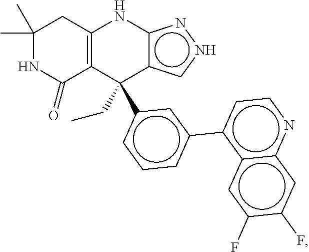 | 948638 |
| 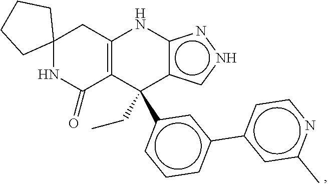 | 948641 |
| 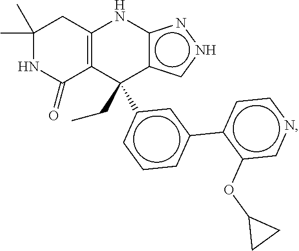 | 948643 |
| 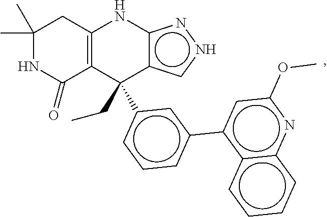 | 948647 |
| 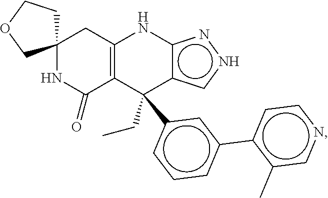 | 948648 |
| 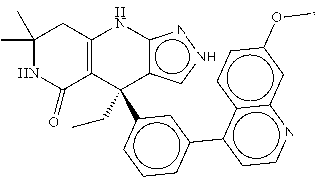 | 948649 |
| 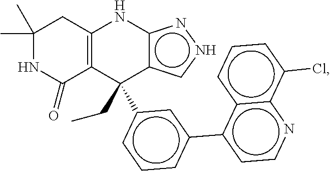 | 948650 |
| 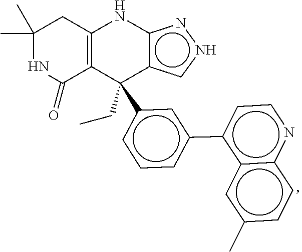 | 948651 |
| 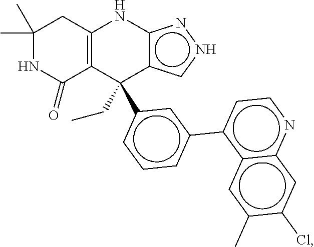 | 948654 |
| 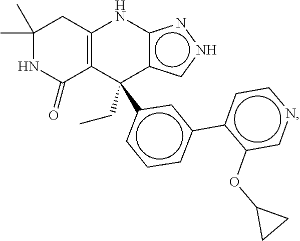 | 948656 |
| 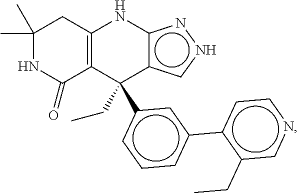 | 948657 |
| 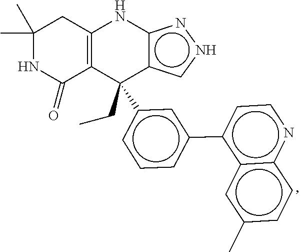 | 948658 |
| 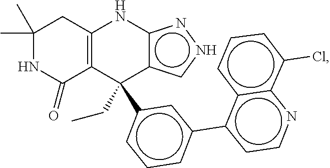 | 948659 |
| 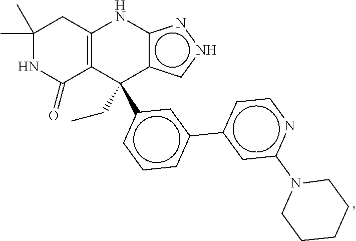 | 948663 |
| 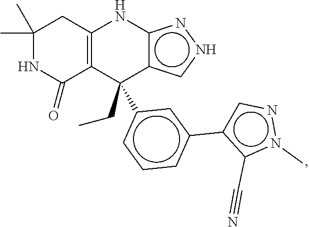 | 948814 |
| 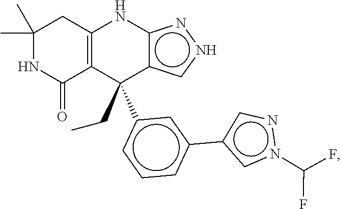 | 948816 |
| 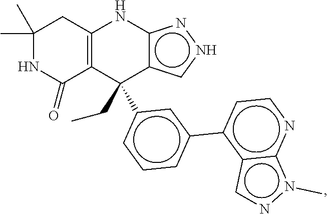 | 948817 |
| 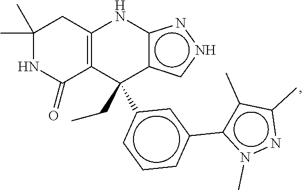 | 948818 |
| 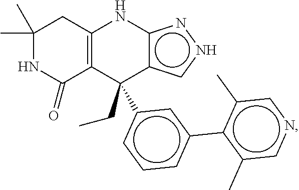 | 948829 |
| 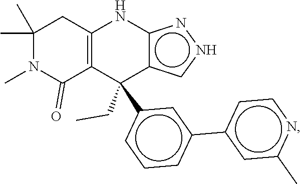 | 949009 |
| 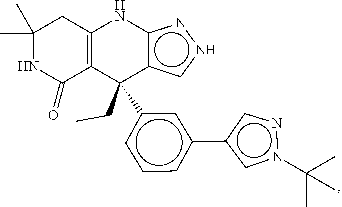 | 949107 |
| 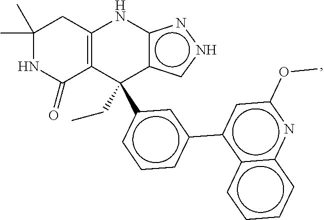 | 949116 |
| 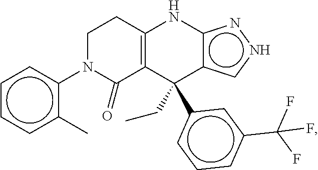 | 949129 |
| 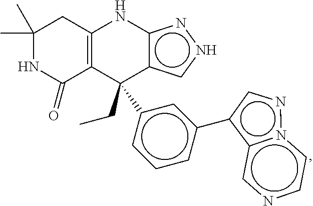 | 949263 |
| 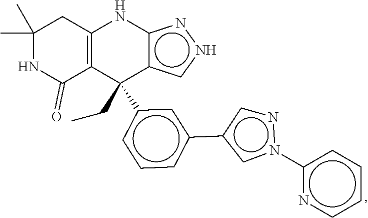 | 949264 |
| 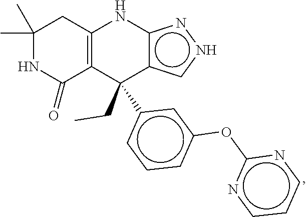 | 949335 |
| 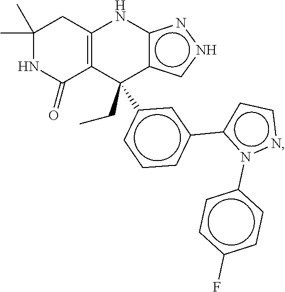 | 949396 |
| 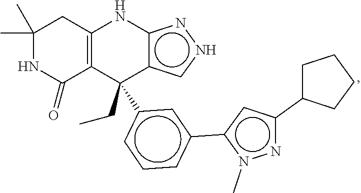 | 949509 |
| 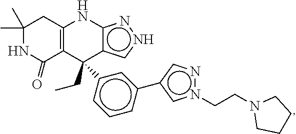 | 949510 |
| 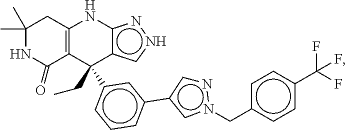 | 949511 |
| 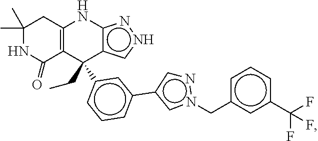 | 949513 |
| 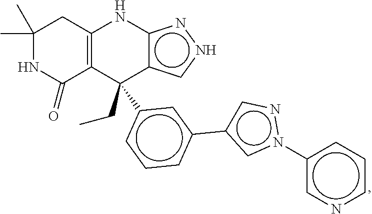 | 949515 |
| 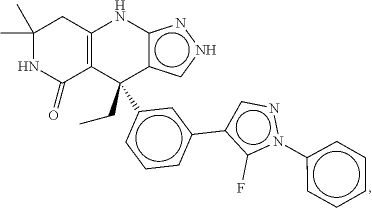 | 949519 |
| 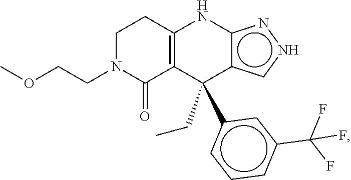 | 949600 |
| 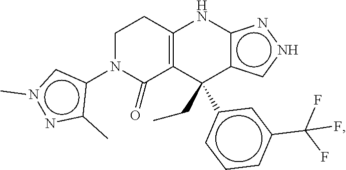 | 949641 |
| 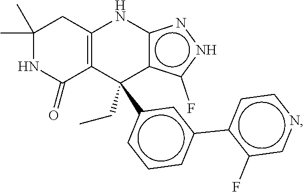 | 949643 |
| 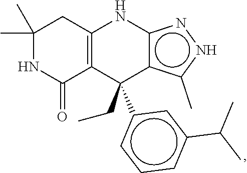 | 949646 |
| 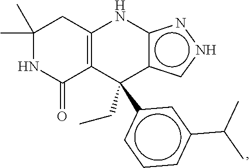 | 949752 |
| 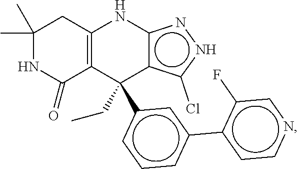 | 949756 |
| 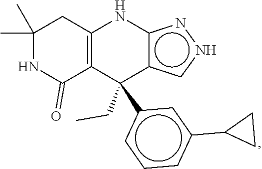 | 950817 |
| 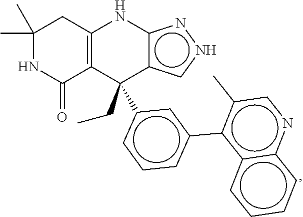 | 950825 |
| 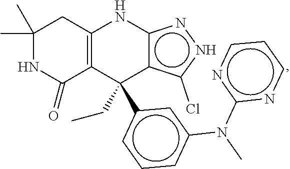 | 951592 |
| 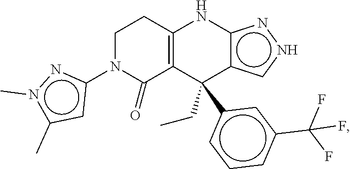 | 951954 |
| 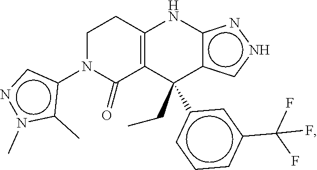 | 952262 |
| 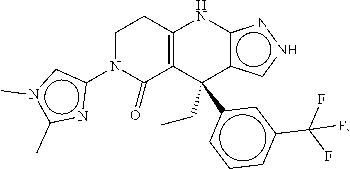 | 952479 |
| 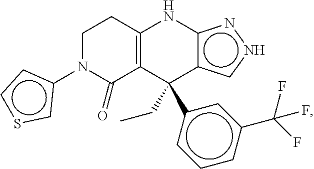 | 952636 |
| 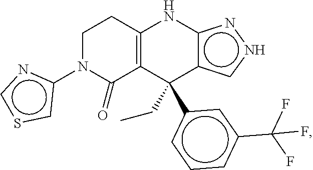 | 952638 |
| 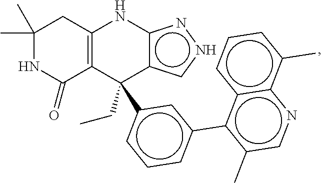 | 952760 |

or

| | |
|---|---|
| 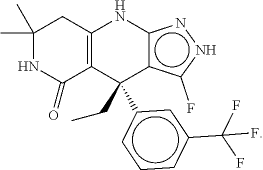 | 957497 |

32. The compound of claim 1, or a pharmaceutically acceptable salt, solvate, hydrate, polymorph, co-crystal, tautomer, stereoisomer, isotopically labeled compound, or prodrug thereof, wherein the compound is any one of the formulae shown in Table 2A:
TABLE 2A

| 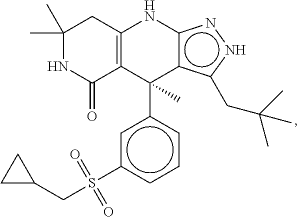 | 108006 |
|---|---|
| 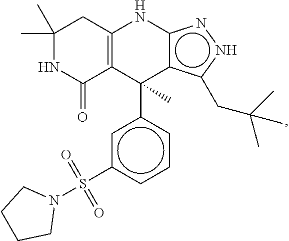 | 108794 |
| or | |
| 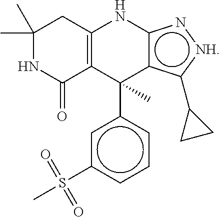 | 114744 |

33. The compound of claim 1, or a pharmaceutically acceptable salt, solvate, hydrate, polymorph, co-crystal, tautomer, stereoisomer, isotopically labeled compound, or prodrug thereof, wherein the compound is not a compound of claim 31 or 32.

34. A compound, or a pharmaceutically acceptable salt, solvate, hydrate, polymorph, co-crystal, tautomer, stereoisomer, isotopically labeled compound, or prodrug thereof, wherein the compound is of Formula II-A or II-B: 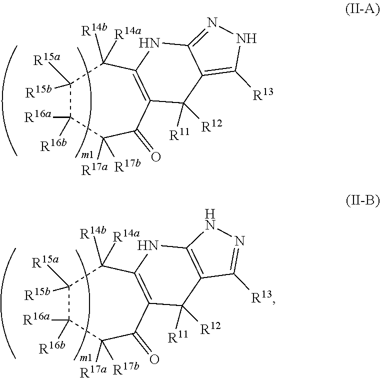
wherein:
each instance of [symbol] is independently a single or double bond;
R¹¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R¹² is optionally substituted alkyl, optionally substituted alkenyl, or optionally substituted alkynyl;
or R¹¹ and R¹² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
R¹³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{B}, —SCN, —SR^{B}, —SSR^{B}, —N₃, —NO, —N(R^{B})₂, —NO₂, —C(═O)R^{B}, —C(═O)OR^{B}, —C(═O)SR^{B}, —C(═O)N(R^{B})₂, —C(═NR^{B})R^{B}, —C(═NR^{B})OR^{B}, —C(═NR^{B})SR^{B}, —C(═NR^{B})N(R^{B})₂, —S(═O)R^{B}, —S(═O)OR^{B}, —S(═O)SR^{B}, —S(═O)N(R^{B})₂, —S(═O)₂R^{B}, —S(═O)₂OR^{B}, —S(═O)₂SR^{B}, —S(═O)₂N(R^{B})₂, —OC(═O)R^{B}, —OC(═O)OR^{B}, —OC(═O)SR^{B}, —OC(═O)N(R^{B})₂, —OC(═NR^{B})R^{B}, —OC(═NR^{B})OR^{B}, —OC(═NR^{B})SR^{B}, —OC(═NR^{B})N(R^{B})₂, —OS(═O)R^{B}, —OS(═O)OR^{B}, —OS(═O)SR^{B}, —OS(═O)N(R^{B})₂, —OS(═O)₂R^{B}, —OS(═O)₂OR^{B}, —OS(═O)₂SR^{B}, —OS(═O)₂N(R^{B})₂, —ON(R^{B})₂, —SC(═O)R^{B}, —SC(═O)OR^{B}, —SC(═O)SR^{B}, —SC(═O)N(R^{B})₂, —SC(═NR^{B})R^{B}, —SC(═NR^{B})OR^{B}, —SC(═NR^{B})SR^{B}, —SC(═NR^{B})N(R^{B})₂, —NR^{B}C(═O)R^{B}, —NR^{B}C(═O)OR^{B}, —NR^{B}C(═O)SR^{B}, —NR^{B}C(═O)N(R^{B})₂, —NR^{B}C(═NR^{B})R^{B}, —NR^{B}C(═NR^{B})OR^{B}, —NR^{B}C(═NR^{B})SR^{B}, —NR^{B}C(═NR^{B})N(R^{B})₂, —NR^{B}S(═O)R^{B}, —NR^{B}S(═O)OR^{B}, —NR^{B}S(═O)SR^{B}, —NR^{B}S(═O)N(R^{B})₂, —NR^{B}S(═O)₂R^{B}, —NR^{B}S(═O)₂OR^{B}, —NR^{B}S(═O)₂SR^{B}, —NR^{B}S(═O)₂N(R^{B})₂, —Si(R^{B})₃, —Si(R^{B})₂OR^{B}, —Si(R^{B})(OR^{B})₂, —Si(OR^{B})₃, —OSi(R^{B})₃, —OSi(R^{B})₂OR^{B}, —OSi(R^{B})(OR^{B})₂, or —OSi(OR^{B})₃;
each of R^{14a}, R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each instance of R^{B} is independently hydrogen, optionally substituted alkyl, optionally substituted alkenyl, optionally substituted alkynyl, optionally substituted heteroalkyl, optionally carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{B} attached to the same intervening atom are joined together with the intervening atom to form an optionally substituted, monocyclic, heterocyclic or heteroaryl ring; and
or R^{14a} and R^{14b} are taken together with their intervening atom to form an optionally substituted, monocyclic, carbocyclic or heterocyclic ring;
or R^{17a} and R^{17b} are taken together with their intervening atom to form an optionally substituted, monocyclic, carbocyclic or heterocyclic ring;
or R^{14b} and R^{17a} are taken together to form optionally substituted alkylene, optionally substituted alkenylene, optionally substituted alkynylene, optionally substituted heteroalkylene, optionally substituted heteroalkenylene, or optionally substituted heteroalkynylene, each of which independently comprises 1, 2, 3, or 4 backbone atoms; and
m1 is 0 or 1;
when m1 is 1:
each of R^{15a}, R^{15b}, R^{16a} and R^{16b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
or R^{14b} and R^{15a} are taken together with their intervening atom to form an optionally substituted, monocyclic, carbocyclic, heterocyclic, aryl, or heteroaryl ring;
or R^{15a} and R^{15b} are taken together with their intervening atom to form an optionally substituted, monocyclic, carbocyclic or heterocyclic ring;
or R^{15b} and R^{16a} are taken together with their intervening atom to form an optionally substituted, monocyclic, carbocyclic, heterocyclic, aryl, or heteroaryl ring;
or R^{16a} and R^{16b} are taken together with their intervening atom to form an optionally substituted, monocyclic, carbocyclic or heterocyclic ring;
or R^{16b} and R^{17a} are taken together with their intervening atom to form an optionally substituted, monocyclic, carbocyclic, heterocyclic, aryl, or heteroaryl ring;
provided that the compound is not of the formula: 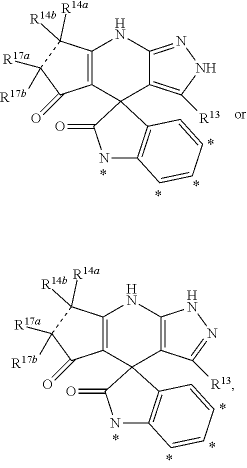
or a pharmaceutically acceptable salt, solvate, hydrate, polymorph, co-crystal, tautomer, stereoisomer, isotopically labeled compound, or prodrug thereof, wherein each instance of the atoms marked with * is independently optionally substituted.

35. The compound of claim 34, or a pharmaceutically acceptable salt, solvate, hydrate, polymorph, co-crystal, tautomer, stereoisomer, isotopically labeled compound, or prodrug thereof, wherein the compound is of the formula:

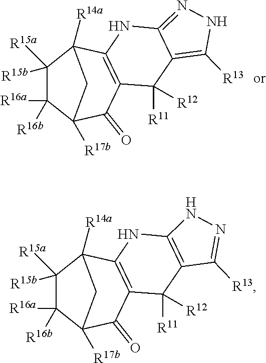

36. The compound of claim 34, or a pharmaceutically acceptable salt, solvate, hydrate, polymorph, co-crystal, tautomer, stereoisomer, isotopically labeled compound, or prodrug thereof, wherein m1 is 0.

37. The compound of claim 34 or 35, or a pharmaceutically acceptable salt, solvate, hydrate, polymorph, co-crystal, tautomer, stereoisomer, isotopically labeled compound, or prodrug thereof, wherein m1 is 1.

38. The compound of any one of claims 34-37, or a pharmaceutically acceptable salt, solvate, hydrate, polymorph, co-crystal, tautomer, stereoisomer, isotopically labeled compound, or prodrug thereof, wherein each instance of [symbol] is a single bond.

39. The compound of any one of claims 34-37, or a pharmaceutically acceptable salt, solvate, hydrate, polymorph, co-crystal, tautomer, stereoisomer, isotopically labeled compound, or prodrug thereof, wherein at least one instance of [symbol] is a double bond.

40. The compound of any one of claims 34-38, or a pharmaceutically acceptable salt, solvate, hydrate, polymorph, co-crystal, tautomer, stereoisomer, isotopically labeled compound, or prodrug thereof, wherein each of R^{15a}, R^{15b}, R^{16a} and R^{16b} is hydrogen.

41. The compound of any one of claims 34-40, or a pharmaceutically acceptable salt, solvate, hydrate, polymorph, co-crystal, tautomer, stereoisomer, isotopically labeled compound, or prodrug thereof, wherein R^{14b} and R^{17a} are taken together to form optionally substituted alkylene or optionally substituted alkenylene, each of which independently comprises 1, 2, 3, or 4 backbone atoms.

42. The compound of any one of claims 34-40, or a pharmaceutically acceptable salt, solvate, hydrate, polymorph, co-crystal, tautomer, stereoisomer, isotopically labeled compound, or prodrug thereof, wherein R^{14a} and R^{14b} are hydrogen.

43. The compound of any one of claims 34-40, or a pharmaceutically acceptable salt, solvate, hydrate, polymorph, co-crystal, tautomer, stereoisomer, isotopically labeled compound, or prodrug thereof, wherein R^{17a} and R^{17b} are independently hydrogen or optionally substituted C₁-C₆ alkyl.

44. The compound of any one of claims 34-43, or a pharmaceutically acceptable salt, solvate, hydrate, polymorph, co-crystal, tautomer, stereoisomer, isotopically labeled compound, or prodrug thereof, wherein R¹¹ is optionally substituted aryl.

45. The compound of claim 44, or a pharmaceutically acceptable salt, solvate, hydrate, polymorph, co-crystal, tautomer, stereoisomer, isotopically labeled compound, or prodrug thereof, wherein R¹¹ is 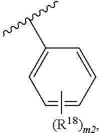
wherein:
each instance of R¹⁸ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃; and
m2 is 0, 1, 2, 3, 4, or 5.

46. The compound of claim 45, or a pharmaceutically acceptable salt, solvate, hydrate, polymorph, co-crystal, tautomer, stereoisomer, isotopically labeled compound, or prodrug thereof, wherein R¹¹ is

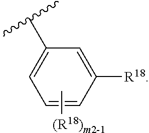

47. The compound of claim 45 or 46, or a pharmaceutically acceptable salt, solvate, hydrate, polymorph, co-crystal, tautomer, stereoisomer, isotopically labeled compound, or prodrug thereof, wherein m2 is 1.

48. The compound of any one of claims 34-47, or a pharmaceutically acceptable salt, solvate, hydrate, polymorph, co-crystal, tautomer, stereoisomer, isotopically labeled compound, or prodrug thereof, wherein R¹² is optionally substituted alkyl.

49. The compound of claim 48, or a pharmaceutically acceptable salt, solvate, hydrate, polymorph, co-crystal, tautomer, stereoisomer, isotopically labeled compound, or prodrug thereof, wherein R¹² is unsubstituted C₁-C₄ alkyl.

50. The compound of any one of claims 34-43, or a pharmaceutically acceptable salt, solvate, hydrate, polymorph, co-crystal, tautomer, stereoisomer, isotopically labeled compound, or prodrug thereof, wherein R¹¹ and R¹² are taken together with their intervening atom to form an optionally substituted, monocyclic carbocyclic ring.

51. The compound of any one of claims 34-43, or a pharmaceutically acceptable salt, solvate, hydrate, polymorph, co-crystal, tautomer, stereoisomer, isotopically labeled compound, or prodrug thereof, wherein R₁ and R¹² are taken together with their intervening atom to form an optionally substituted, monocyclic heterocyclic ring.

52. The compound of claim 51, or a pharmaceutically acceptable salt, solvate, hydrate, polymorph, co-crystal, tautomer, stereoisomer, isotopically labeled compound, or prodrug thereof, wherein R¹¹ and R¹² are taken together with their intervening atom to form an optionally substituted tetrahydropyranyl.

53. The compound of any one of claims 34-52, or a pharmaceutically acceptable salt, solvate, hydrate, polymorph, co-crystal, tautomer, stereoisomer, isotopically labeled compound, or prodrug thereof, wherein R¹³ is hydrogen, optionally substituted C₁-C₆ alkyl, or halogen.

54. The compound of claim 53, or a pharmaceutically acceptable salt, solvate, hydrate, polymorph, co-crystal, tautomer, stereoisomer, isotopically labeled compound, or prodrug thereof, wherein R¹³ is hydrogen.

55. The compound of claim 34, or a pharmaceutically acceptable salt, solvate, hydrate, polymorph, co-crystal, tautomer, stereoisomer, isotopically labeled compound, or prodrug thereof, wherein the compound is any one of the formulae shown in Table 3:
TABLE 3

| | |
|---|---|
| 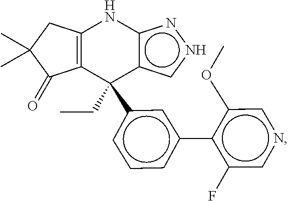 | 952562 |
| 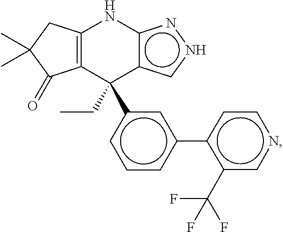 | 952757 |
| 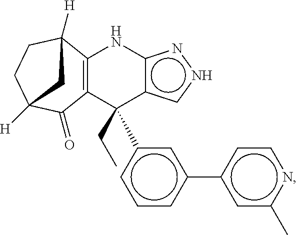 | 943921 |
| 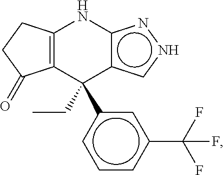 | 951566 |
| 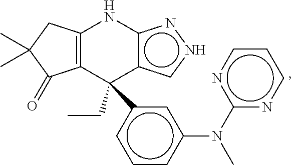 | 952205 |
| 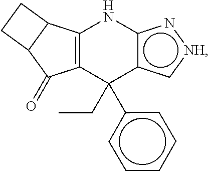 | 861716 |
| 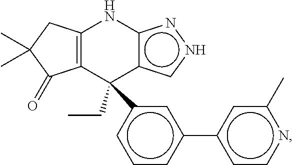 | 950283 |
| 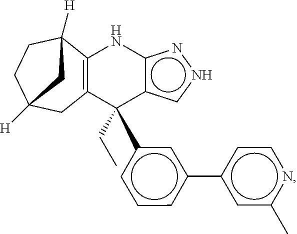 | 943920 |
| 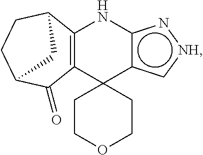 | 912081 |
| 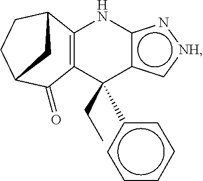 | 791270 |
| 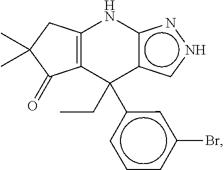 | 949805 |
| 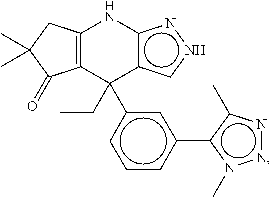 | 950815 |
| 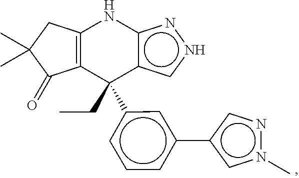 | 950819 |
| 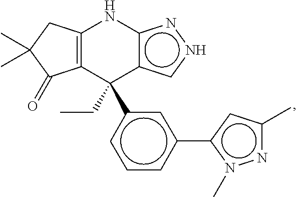 | 950818 |
| 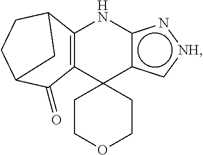 | 901464 |
| 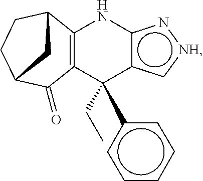 | 791269 |
| 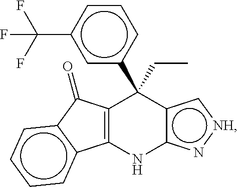 | 949742 |
| 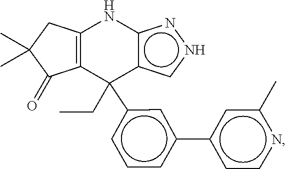 | 949840 |
| 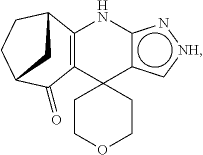 | 912080 |
| 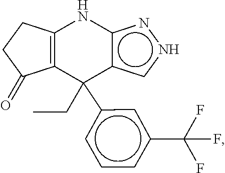 | 949639 |
| 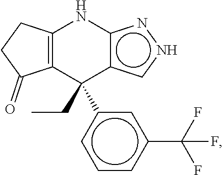 | 951565 |
| 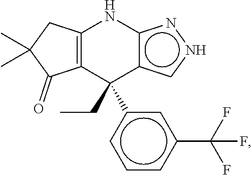 | 950272 |
| 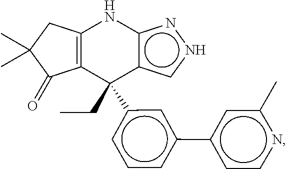 | 950282 |
| 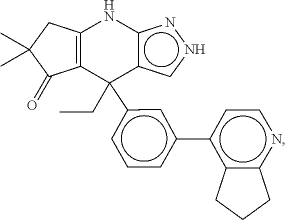 | 952557 |
| 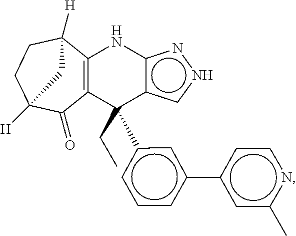 | 943917 |
| 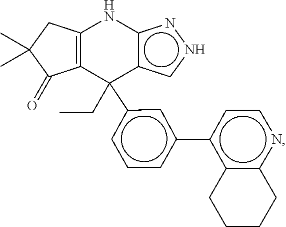 | 952752 |
| 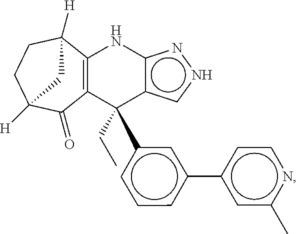 | 943916 |
| 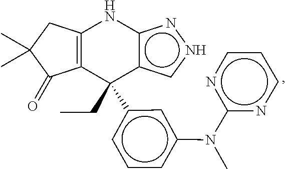 | 952212 |
| 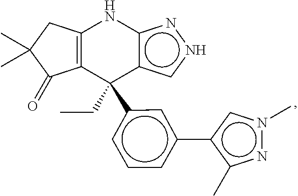 | 951702 |
| 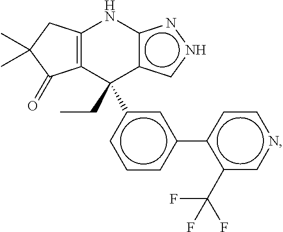 | 952759 |
| 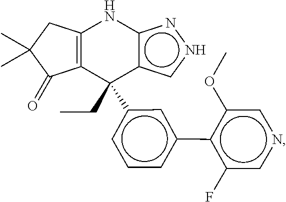 | 952564 |
| 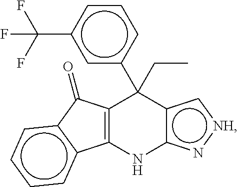 | 948205 |
| 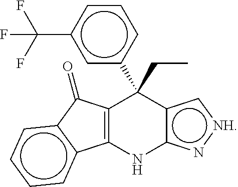 | 949743 |

56. The compound of claim 34, or a pharmaceutically acceptable salt, solvate, hydrate, polymorph, co-crystal, tautomer, stereoisomer, isotopically labeled compound, or prodrug thereof, wherein the compound is any one of the formulae shown in Table 4:
TABLE 4

| | |
|---|---|
| 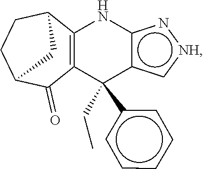 | 791266 |
| 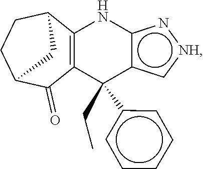 | 791268 |
| 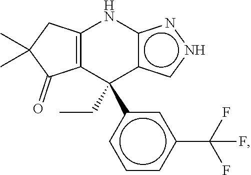 | 950273 |
| 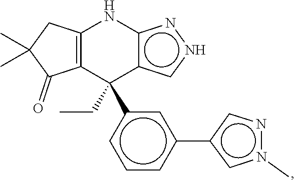 | 950826 |
| 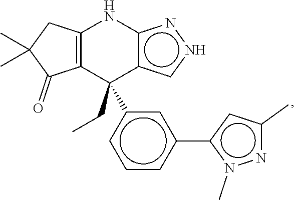 | 950827 |
| 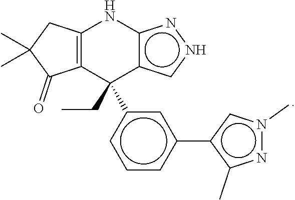 | 951710 |

57. The compound of claim 34, or a pharmaceutically acceptable salt, solvate, hydrate, polymorph, co-crystal, tautomer, stereoisomer, isotopically labeled compound, or prodrug thereof, wherein the compound is not a compound of claim 56.

58. A compound, or a pharmaceutically acceptable salt, solvate, hydrate, polymorph, co-crystal, tautomer, stereoisomer, isotopically labeled compound, or prodrug thereof, wherein the compound is of Formula III-A or III-B: 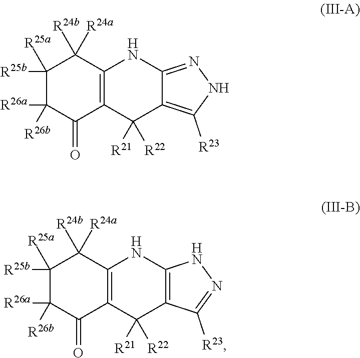
wherein:
R²¹ and R²² are taken together with their intervening atom to form a substituted monocyclic carbocyclic ring or an optionally substituted, monocyclic heterocyclic ring;
optionally two substituents on the monocyclic carbocyclic ring or monocyclic heterocyclic ring are taken together with their intervening atom(s) to form an additional optionally substituted, monocyclic, carbocyclic or heterocyclic ring;
R²³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{C}, —SCN, —SR^{C}, —SSR^{C}, —N₃, —NO, —N(R^{C})₂, —NO₂, —C(═O)R^{C}, —C(═O)OR^{C}, —C(═O)SR^{C}, —C(═O)N(R^{C})₂, —C(═NR^{C})R^{C}, —C(═NR^{C})OR^{C}, —C(═NR^{C})SR^{C}, —C(═NR^{C}N(R^{C})₂, —S(═O)R^{C}, —S(═O)OR^{C}, —S(═O)SR^{C}, —S(═O)N(R^{C})₂, —S(═O)₂R^{C}, —S(═O)₂OR^{C}, —S(═O)₂SR^{C}, —S(═O)₂N(R^{C})₂, —OC(═O)R^{C}, —OC(═O)OR^{C}, —OC(═O)SR^{C}, —OC(═O)N(R^{C})₂, —OC(═NR^{C})R^{C}, —OC(═NR^{C})OR^{C}, —OC(═NR^{C})SR^{C}, —OC(═NR^{C})N(R^{C})₂, —OS(═O)R^{C}, —OS(═O)OR^{C}, —OS(═O)SR^{C}, —OS(═O)N(R^{C})₂, —OS(═O)₂R^{C}, —OS(═O)₂OR^{C}, —OS(═O)₂SR^{C}, —OS(═O)N(R^{C})₂, —ON(R^{C})₂, —SC(═O)R^{C}, —SC(═O)OR^{C}, —SC(═O)SR^{C}, —SC(═O)N(R^{C})₂, —SC(═NR^{C})R^{C}, —SC(═NR^{C})OR^{C}, —SC(═NR^{C})SR^{C}, —SC(═NR^{C})N(R^{C})₂, —NR^{C}C(═O)R^{C}, —NR^{C}C(═O)OR^{C}, —NR^{C}C(═O)SR^{C}, —NR^{C}C(═O)N(R^{C})₂, —NR^{C}C(═NR^{C})R^{C}, —NR^{C}C(═NR^{C})OR^{C}, —NR^{C}C(NR^{C})SR^{C}, —NR^{C}C(═NR^{C}N(R^{C})₂, —NR^{C}S(O)R^{C}, —NR^{C}S(O)OR^{C}, —NR^{C}S(═O)SR^{C}, —NR^{C}S(═O)N(R^{C})₂, —NR^{C}S(═O)₂R^{C}, —NR^{C}S(═O)₂OR^{C}, —NR^{C}S(═O)₂SR^{C}, —NR^{C}S(═O)₂N(R^{C})₂, —Si(R^{C})₃, —Si(R^{C})₂OR^{C}, —Si(R^{C})(OR^{C})₂, —Si(OR^{C})₃, —OSi(R^{C})₃, —OSi(R^{C})₂OR^{C}, —OSi(R^{C})(OR^{C})₂, or —OSi(OR^{C})₃;
each of R^{24a}, R^{24b}, R^{25a}, R^{25b}, R^{26a}, and R^{26b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl; and
each instance of R^{C} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{C} attached to the same intervening atom are joined together with the intervening atom to form an optionally substituted, monocyclic, heterocyclic or heteroaryl ring;
or R^{24a} and R^{24b} are taken together with their intervening atom to form an optionally substituted, monocyclic, carbocyclic or heterocyclic ring;
or R^{24b} and R^{25a} are taken together with their intervening atom to form an optionally substituted, monocyclic, carbocyclic or heterocyclic ring;
or R^{25a} and R^{25b} are taken together with their intervening atom to form an optionally substituted, monocyclic, carbocyclic or heterocyclic ring;
or R^{25b} and R^{26a} are taken together with their intervening atom to form an optionally substituted, monocyclic, carbocyclic or heterocyclic ring;
or R^{26a} and R^{26b} are taken together with their intervening atom to form an optionally substituted, monocyclic, carbocyclic or heterocyclic ring;
provided that the compound is not of the formula: 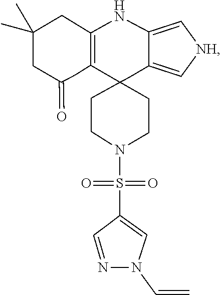 [SMILES: C=CN1C=C(S(=O)(=O)N2CCC3(CC2)Cc2c[nH]cc2NC2=C3CC(=O)CC(C)(C)C2)CC=N1]
or a pharmaceutically acceptable salt, solvate, hydrate, polymorph, co-crystal, tautomer, stereoisomer, isotopically labeled compound, or prodrug thereof.

59. The compound of claim 58, or a pharmaceutically acceptable salt, solvate, hydrate, polymorph, co-crystal, tautomer, stereoisomer, isotopically labeled compound, or prodrug thereof, wherein each of R^{26a} and R^{26b} is independently hydrogen, halogen, or optionally substituted alkyl.

60. The compound of claim 59, or a pharmaceutically acceptable salt, solvate, hydrate, polymorph, co-crystal, tautomer, stereoisomer, isotopically labeled compound, or prodrug thereof, wherein at least one of R^{26a} and R^{26b} is fluorine.

61. The compound of any one of claims 58-60, or a pharmaceutically acceptable salt, solvate, hydrate, polymorph, co-crystal, tautomer, stereoisomer, isotopically labeled compound, or prodrug thereof, wherein each of R^{25a} and R^{25b} is independently hydrogen, halogen, optionally substituted alkyl, or optionally substituted aryl.

62. The compound of claim 61, or a pharmaceutically acceptable salt, solvate, hydrate, polymorph, co-crystal, tautomer, stereoisomer, isotopically labeled compound, or prodrug thereof, wherein R^{25a} and R^{25b} are —CH₃.

63. The compound of any one of claims 58-60, or a pharmaceutically acceptable salt, solvate, hydrate, polymorph, co-crystal, tautomer, stereoisomer, isotopically labeled compound, or prodrug thereof, wherein R^{25a} and R^{25b} are taken together with their intervening atom to form an optionally substituted, monocyclic, carbocyclic ring.

64. The compound of any one of claims 58-60, or a pharmaceutically acceptable salt, solvate, prodrug thereof, wherein R^{25a} and R²Sb are taken together with their intervening atom to form an optionally substituted, monocyclic, heterocyclic ring.

65. The compound of any one of claims 58-64, or a pharmaceutically acceptable salt, solvate, hydrate, polymorph, co-crystal, tautomer, stereoisomer, isotopically labeled compound, or prodrug thereof, wherein each of R^{24a} and R^{24b} is independently hydrogen, halogen, or optionally substituted alkyl.

66. The compound of claim 65, or a pharmaceutically acceptable salt, solvate, hydrate, polymorph, co-crystal, tautomer, stereoisomer, isotopically labeled compound, or prodrug thereof, wherein at least one of R^{24a} and R^{24b} is fluorine.

67. The compound of any one of claims 58-66, or a pharmaceutically acceptable salt, solvate, hydrate, polymorph, co-crystal, tautomer, stereoisomer, isotopically labeled compound, or prodrug thereof, wherein R²¹ and R²² are taken together with their intervening atom to form a substituted monocyclic C₄₋₇ carbocyclic ring.

68. The compound of any one of claims 58-66, or a pharmaceutically acceptable salt, solvate, hydrate, polymorph, co-crystal, tautomer, stereoisomer, isotopically labeled compound, or prodrug thereof, wherein R²¹ and R²² are taken together with their intervening atom to form an optionally substituted, 4- to 7-membered monocyclic heterocyclic ring.

69. The compound of claim 68, or a pharmaceutically acceptable salt, solvate, hydrate, polymorph, co-crystal, tautomer, stereoisomer, isotopically labeled compound, or prodrug thereof, wherein R²¹ and R²² are taken together with their intervening atom to form an optionally substituted, 5- to 6-membered monocyclic heterocyclic ring.

70. The compound of claim 68 or 69, or a pharmaceutically acceptable salt, solvate, hydrate, polymorph, co-crystal, tautomer, stereoisomer, isotopically labeled compound, or prodrug thereof, wherein the monocyclic heterocyclic ring comprises O and/or N heteroatom(s) as the only heteroatoms in the heterocyclic ring.

71. The compound of any one of claims 68-70, or a pharmaceutically acceptable salt, solvate, hydrate, polymorph, co-crystal, tautomer, stereoisomer, isotopically labeled compound, or prodrug thereof, wherein R²¹ and R²² are taken together with their intervening atom to form an optionally substituted piperidine ring.

72. The compound of claim 71, or a pharmaceutically acceptable salt, solvate, hydrate, polymorph, co-crystal, tautomer, stereoisomer, isotopically labeled compound, or prodrug thereof, wherein R²¹ and R²² are taken together with their intervening atom to form 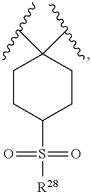
wherein R²⁸ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a sulfur protecting group.

73. The compound of any one of claims 68-71, or a pharmaceutically acceptable salt, solvate, hydrate, polymorph, co-crystal, tautomer, stereoisomer, isotopically labeled compound, or prodrug thereof, wherein two substituents on the monocyclic heterocyclic ring are taken together with their intervening atom(s) to form an additional 4- to 7-membered optionally substituted, monocyclic, carbocyclic or heterocyclic ring.

74. The compound of claim 73, or a pharmaceutically acceptable salt, solvate, hydrate, polymorph, co-crystal, tautomer, stereoisomer, isotopically labeled compound, or prodrug thereof, wherein R²¹ and R²² are taken together with their intervening atom to form 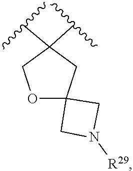
wherein R²⁹ is hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group.

75. The compound of any one of claims 68-70, or a pharmaceutically acceptable salt, solvate, hydrate, polymorph, co-crystal, tautomer, stereoisomer, isotopically labeled compound, or prodrug thereof, wherein R²¹ and R²² are taken together with their intervening atom to form optionally substituted tetrahydropyranyl.

76. The compound of any one of claims 58-75, or a pharmaceutically acceptable salt, solvate, hydrate, polymorph, co-crystal, tautomer, stereoisomer, isotopically labeled compound, or prodrug thereof, wherein R²³ is hydrogen, optionally substituted C₁-C₆ alkyl, or halogen.

77. The compound of claim 76, or a pharmaceutically acceptable salt, solvate, hydrate, polymorph, co-crystal, tautomer, stereoisomer, isotopically labeled compound, or prodrug thereof, wherein R²³ is hydrogen, fluorine, —CH₃, —CH₂F, —CHF₂, or —CF₃.

78. The compound of claim 58, or a pharmaceutically acceptable salt, solvate, hydrate, polymorph, co-crystal, tautomer, stereoisomer, isotopically labeled compound, or prodrug thereof, wherein the compound is any one of the formulae shown in Table 5:
TABLE 5

| | |
|---|---|
| 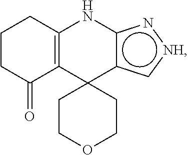 | 884111 |
| 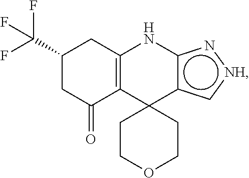 | 901477 |
| 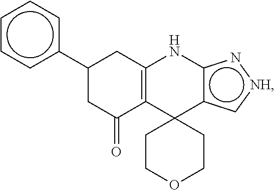 | 893309 |
| 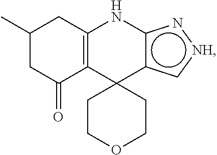 | 884112 |
| 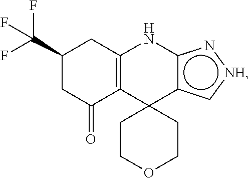 | 901478 |
| 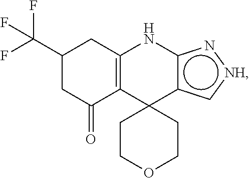 | 889572 |
| 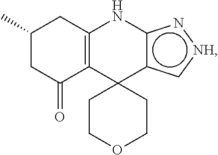 | 912078 |
| 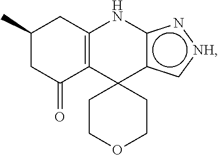 | 912079 |
| 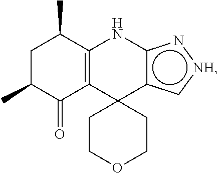 | 893316 |
| 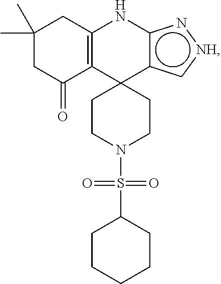 | 913128 |
| 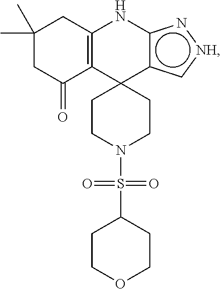 | 913127 |
| 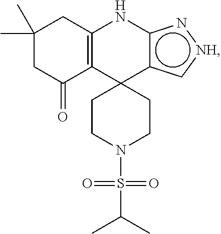 | 913129 |
| 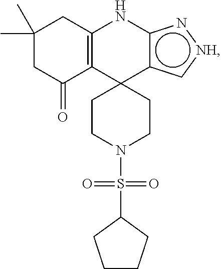 | 909206 |
| 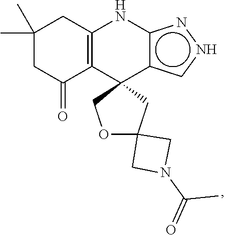 | 926861 |
| 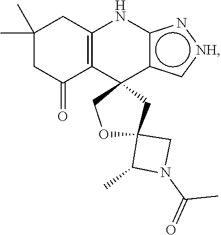 | 948437 |
| 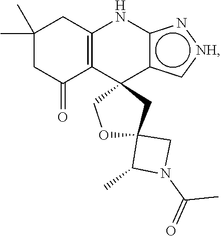 | 948439 |
| 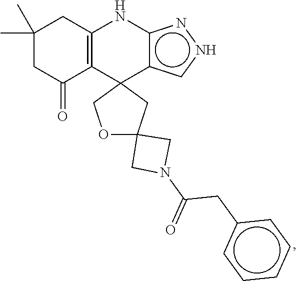 | 947306 |
| 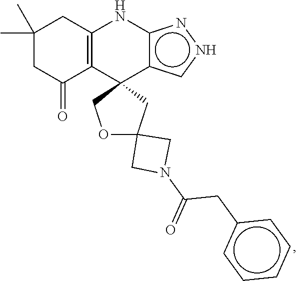 | 948037 |
| 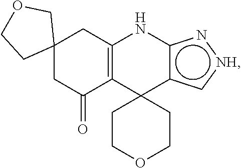 | 889573 |
| 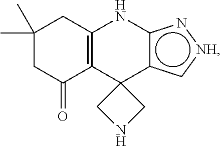 | 912075 |
| 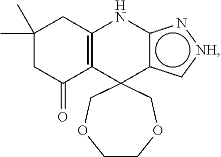 | 912094 |
| 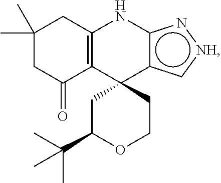 | 912113 |
| 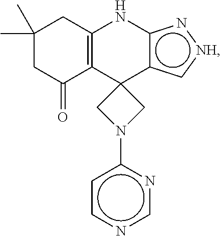 | 909209 |
| 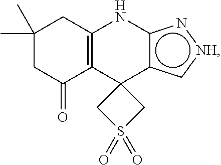 | 912170 |
| 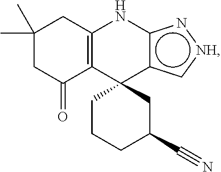 | 946078 |
| 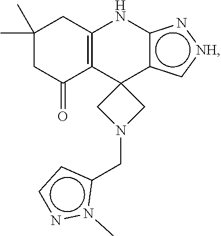 | 932653 |
| 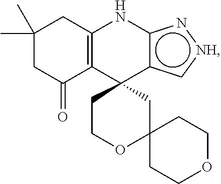 | 942301 |
| 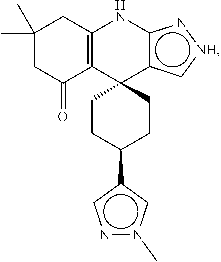 | 949323 |
| 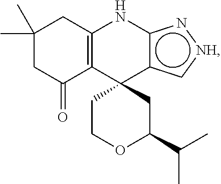 | 884013 |
| 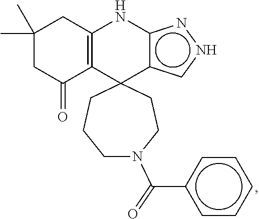 | 889577 |
| 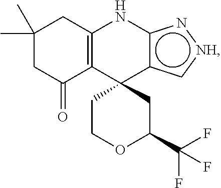 | 884023 |
| 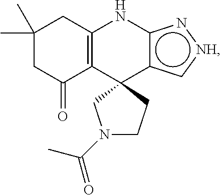 | 889583 |
| 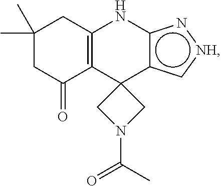 | 893314 |
| 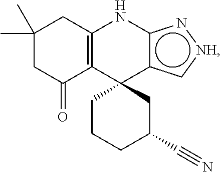 | 946080 |
| 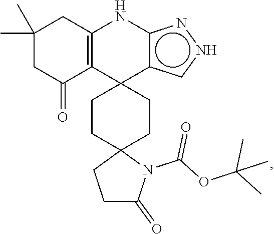 | 912217 |
| 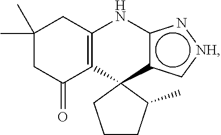 | 874319 |
| 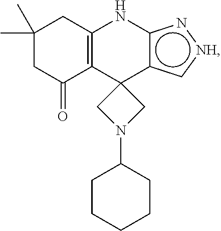 | 929446 |
| 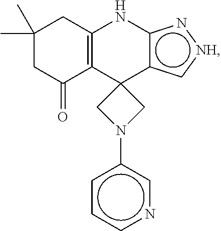 | 947813 |
| 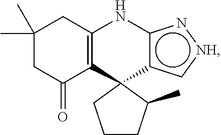 | 874320 |
| 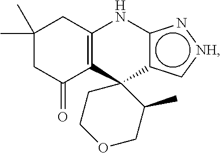 | 924309 |
| 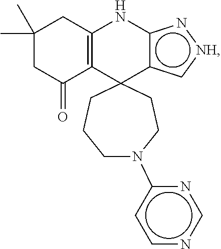 | 917145 |
| 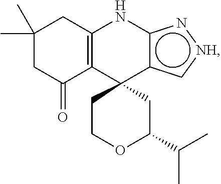 | 884014 |
| 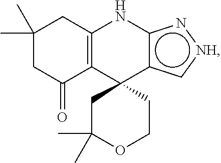 | 924298 |
| 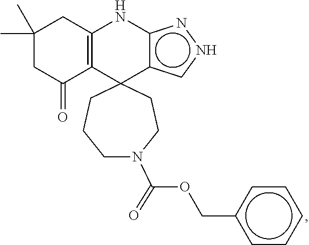 | 884016 |
| 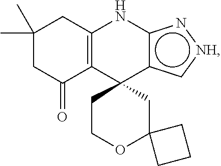 | 897755 |
| 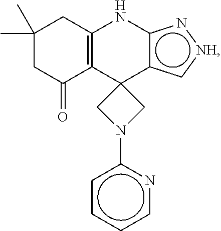 | 945477 |
| 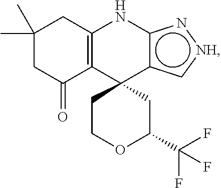 | 884024 |
| 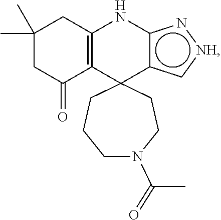 | 889575 |
| 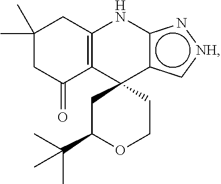 | 912106 |
| 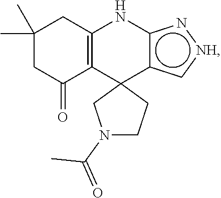 | 826763 |
| 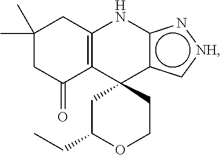 | 907328 |
| 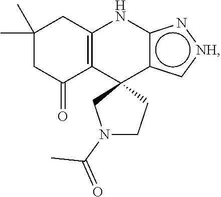 | 889584 |
| 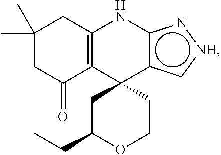 | 907329 |
| 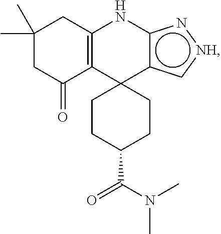 | 945395 |
| 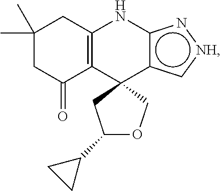 | 913184 |
| 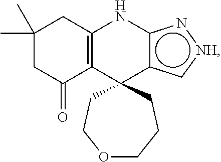 | 874536 |
| 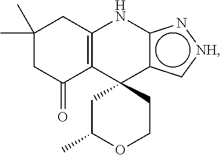 | 899559 |
| 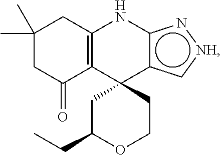 | 924311 |
| 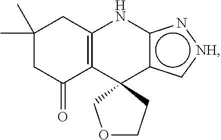 | 877314 |
| 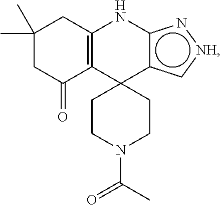 | 826649 |
| 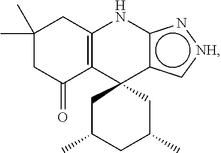 | 884113 |
| 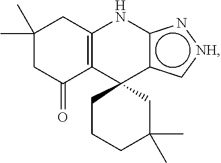 | 883846 |
| 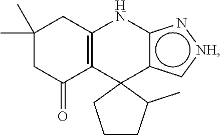 | 826765 |
| 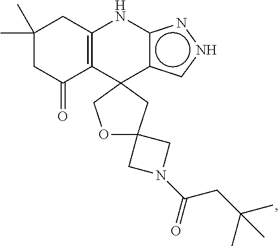 | 947301 |
| 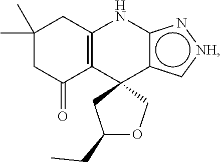 | 913214 |
| 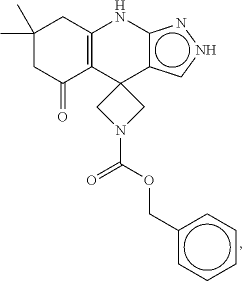 | 889568 |
| 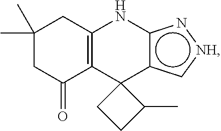 | 949640 |
| 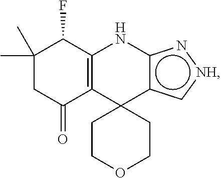 | 883833 |
| 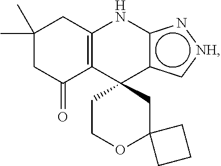 | 897756 |
| 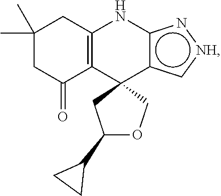 | 913185 |
| 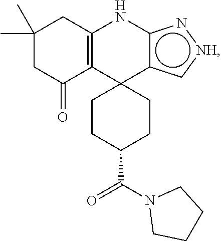 | 945394 |
| 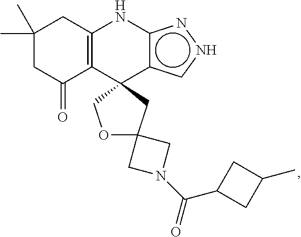 | 947732 |
| 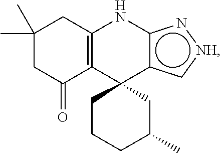 | 874537 |
| 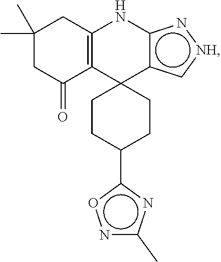 | 943895 |
| 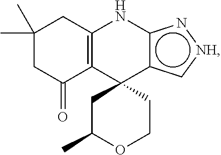 | 899560 |
| 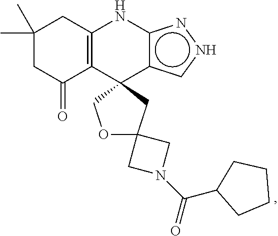 | 943958 |
| 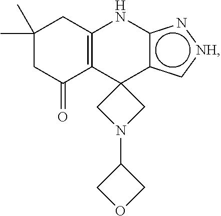 | 912076 |
| 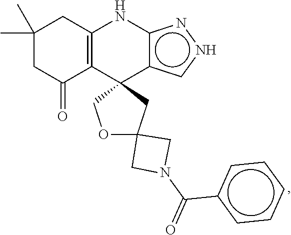 | 943956 |
| 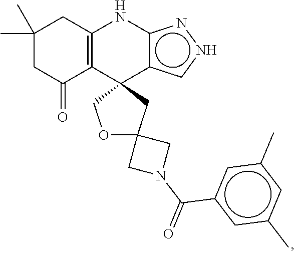 | 948222 |
| 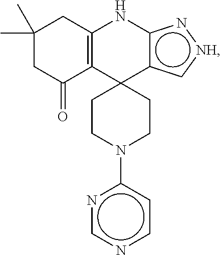 | 874529 |
| 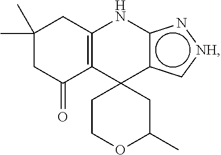 | 883794 |
| 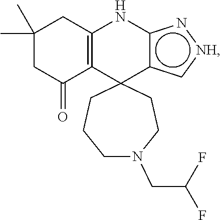 | 889574 |
| 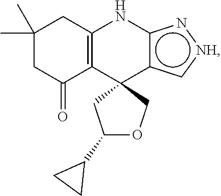 | 913185 |
| 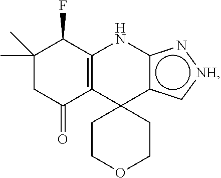 | 883834 |
| 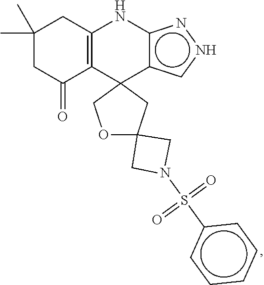 | 913002 |
| 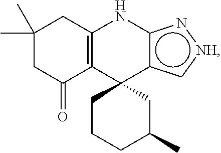 | 893319 |
| 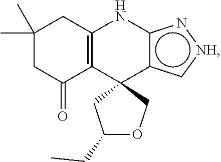 | 913215 |
| 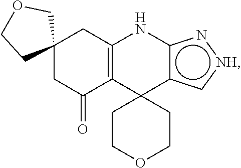 | 901475 |
| 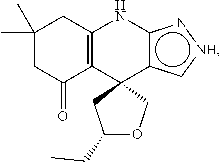 | 913213 |
| 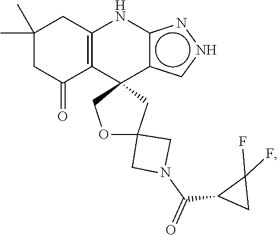 | 948217 |
| 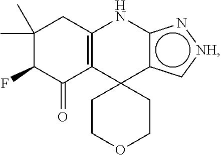 | 883836 |
| 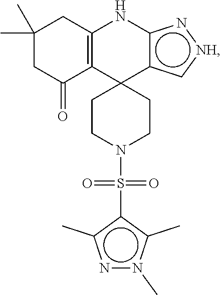 | 947257 |
| 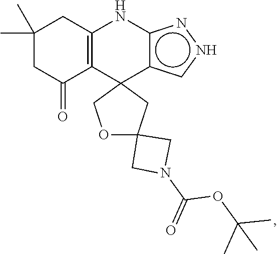 | 911083 |
| 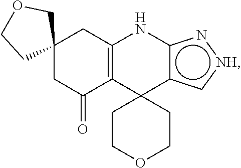 | 901476 |
| 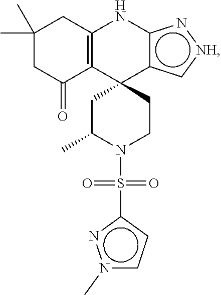 | 917160 |
| 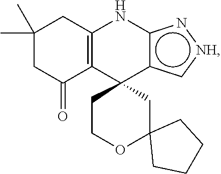 | 907326 |
| 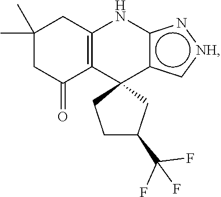 | 952434 |
| 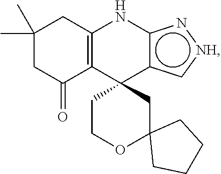 | 907327 |
| 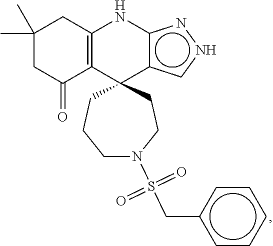 | 945483 |
| 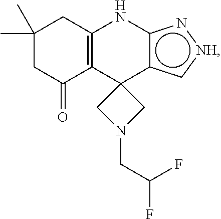 | 912073 |
| 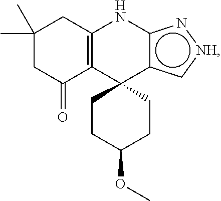 | 884120 |
| 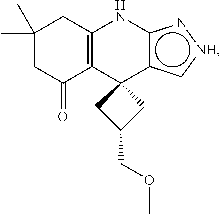 | 912173 |
| 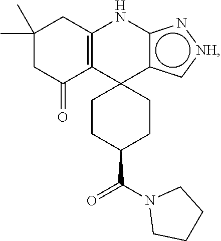 | 945396 |
| 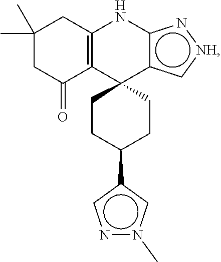 | 949322 |
| 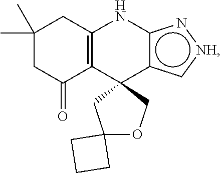 | 917129 |
| 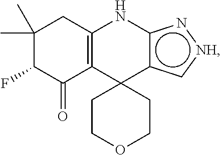 | 993935 |
| 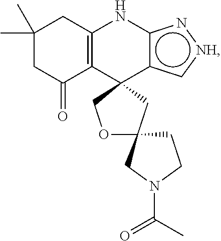 | 948320 |
| 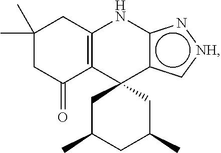 | 884114 |
| 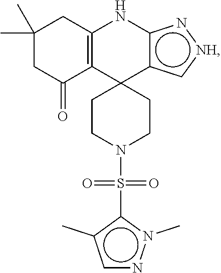 | 945658 |
| 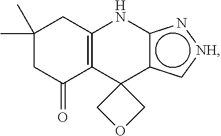 | 889569 |
| 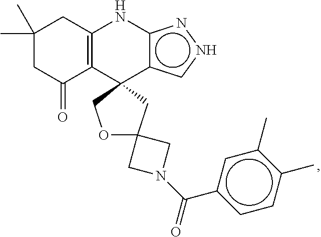 | 948039 |
| 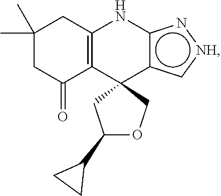 | 913442 |
| 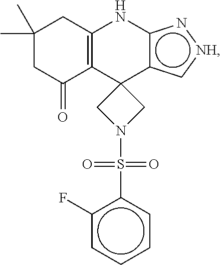 | 913263 |
| 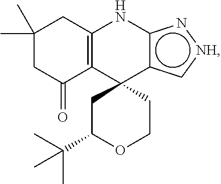 | 912112 |
| 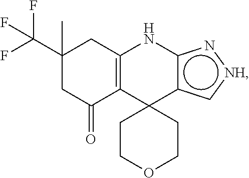 | 901458 |
| 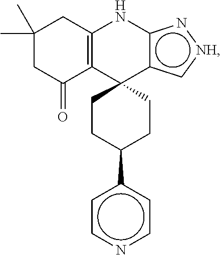 | 949320 |
| 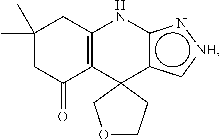 | 826713 |
| 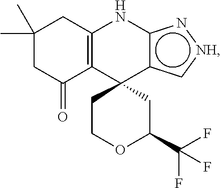 | 884022 |
| 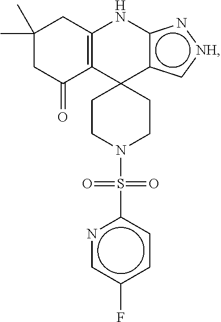 | 947251 |
| 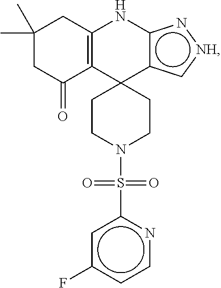 | 947262 |
| 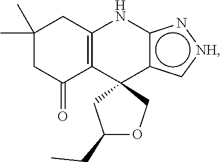 | 913216 |
| 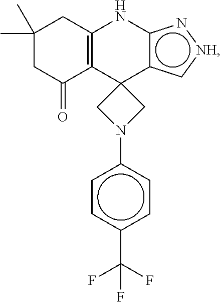 | 945478 |
| 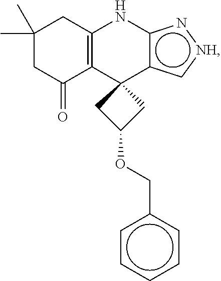 | 909210 |
| 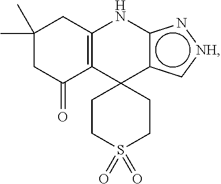 | 896013 |
| 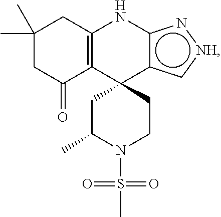 | 918315 |
| 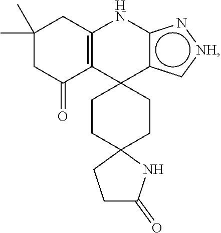 | 913056 |
| 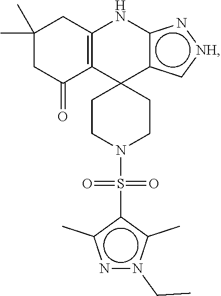 | 945679 |
| 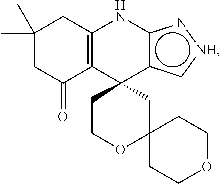 | 924306 |
| 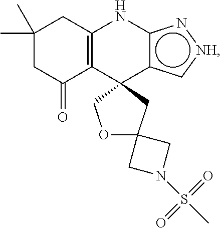 | 926823 |
| 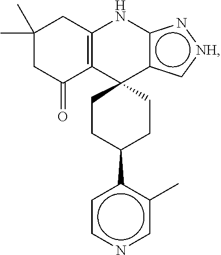 | 949318 |
| 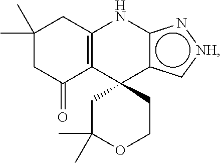 | 924299 |
| 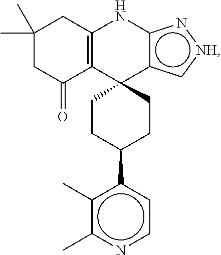 | 950477 |
| 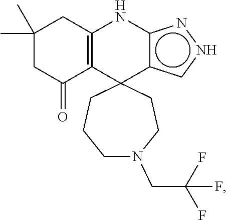 | 889576 |
| 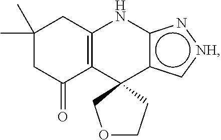 | 877313 |
| 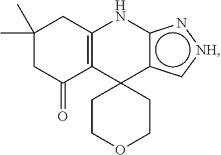 | 826648 |
| 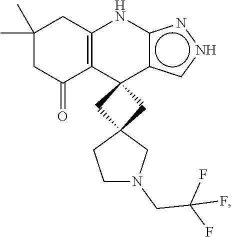 | 913138 |
| 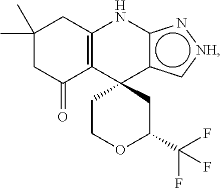 | 884021 |
| 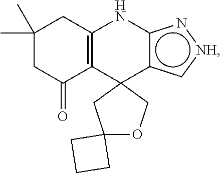 | 911973 |
| 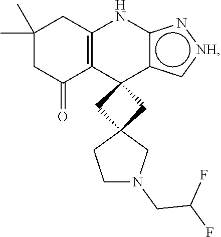 | 913139 |
| 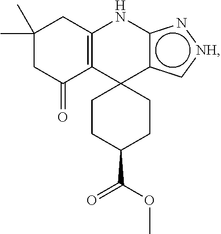 | 918270 |
| 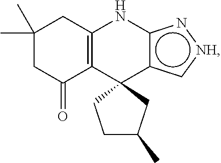 | 883840 |
| 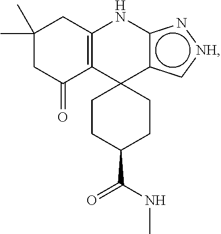 | 945397 |
| 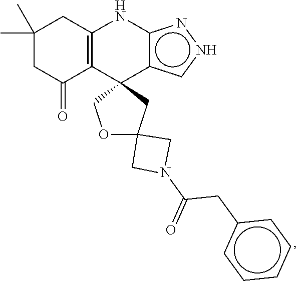 | 948036 |
| 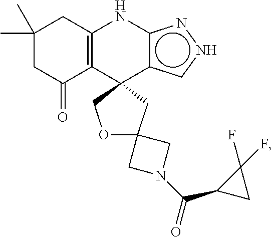 | 948221 |
| 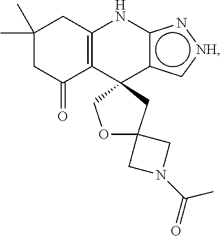 | 926872 |
| 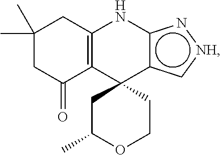 | 924308 |
| 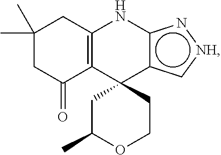 | 924307 |
| 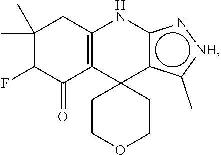 | 912072 |
| 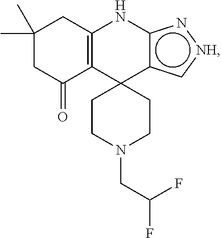 | 883962 |
| 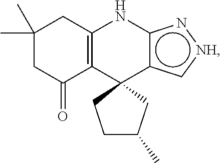 | 883841 |
| 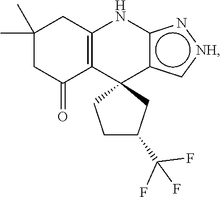 | 952437 |
| 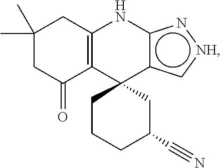 | 945906 |
| 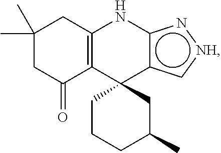 | 893318 |
| 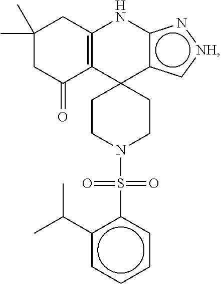 | 947473 |
| 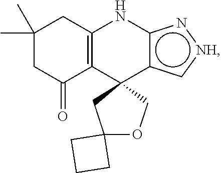 | 917128 |
| 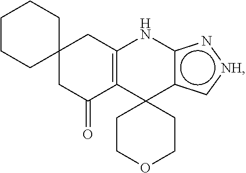 | 893307 |
| 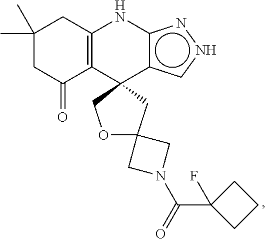 | 947736 |
| 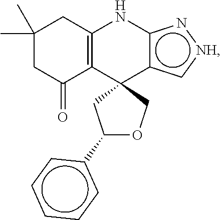 | 946072 |
| 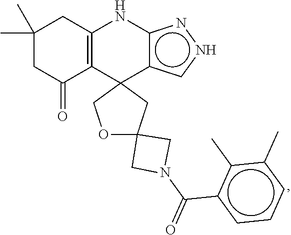 | 947284 |
| 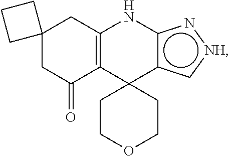 | 909208 |
| 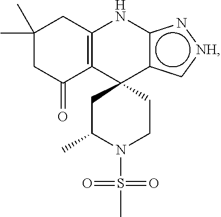 | 918316 |
| 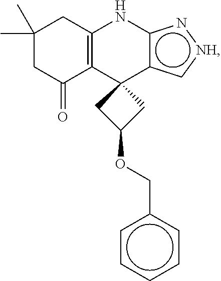 | 909211 |
| 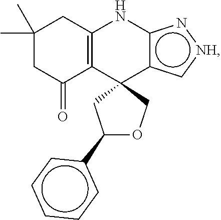 | 946073 |
| 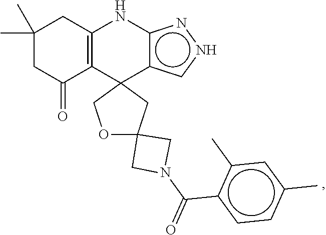 | 947294 |
| 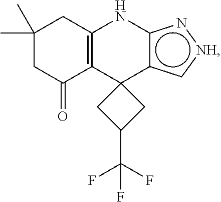 | 874527 |
| 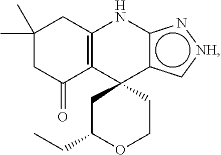 | 924715 |
| 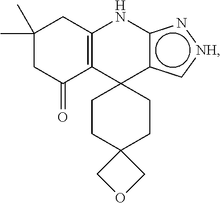 | 883977 |
| 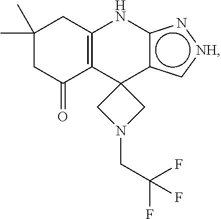 | 912074 |
| 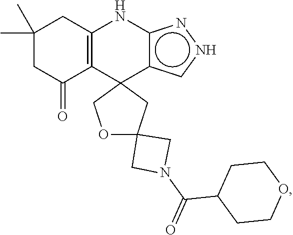 | 945407 |
| 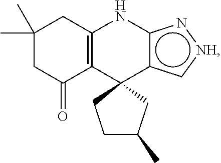 | 897751 |
| 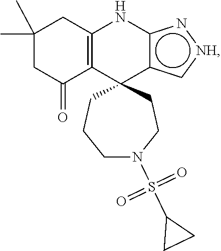 | 932659 |
| 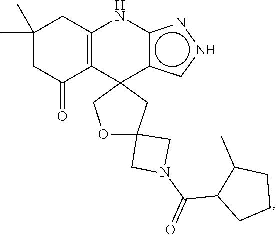 | 947274 |
| 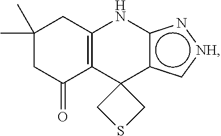 | 889567 |
| 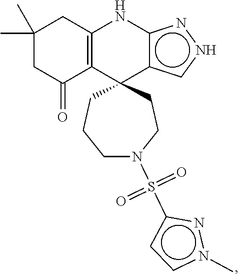 | 932660 |
| 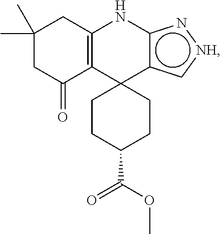 | 918277 |
| 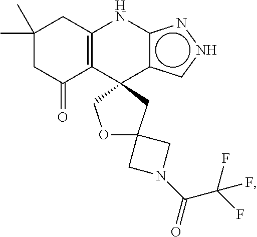 | 948156 |
| 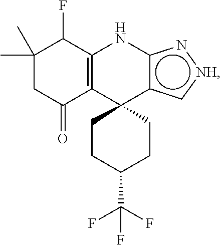 | 917136 |
| 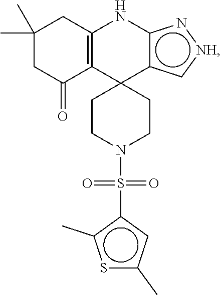 | 945674 |
| 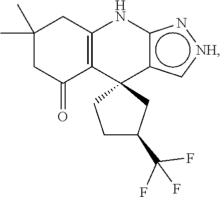 | 952435 |
| 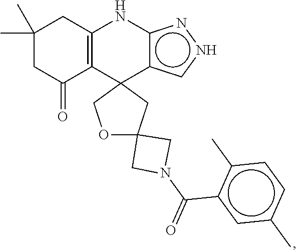 | 947285 |
| 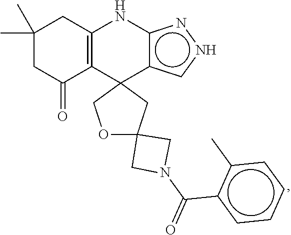 | 947287 |
| 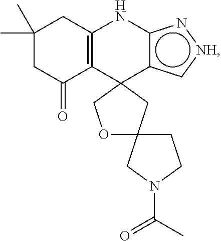 | 948040 |
| 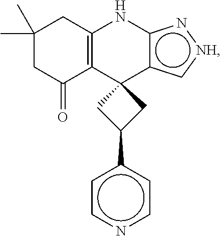 | 889664 |
| 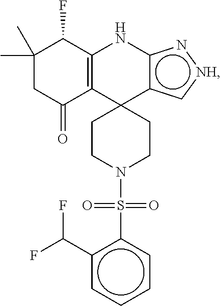 | 951839 |
| 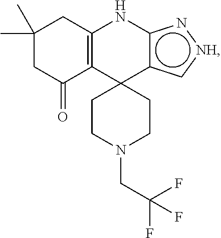 | 874528 |
| 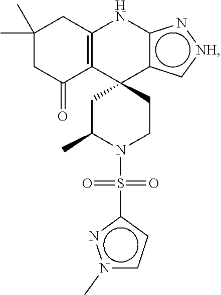 | 926832 |
| 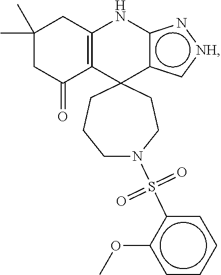 | 917142 |
| 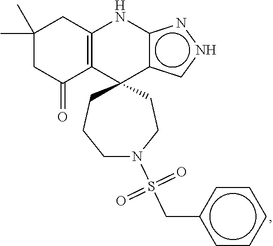 | 945473 |
| 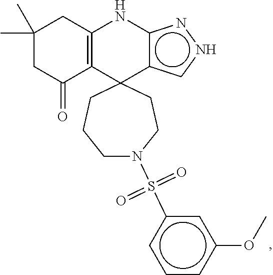 | 917140 |
| 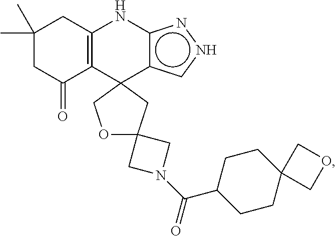 | 944075 |
| 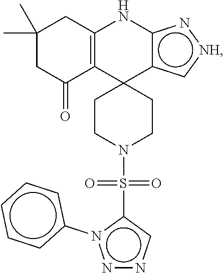 | 947466 |
| 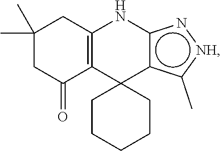 | 883865 |
| 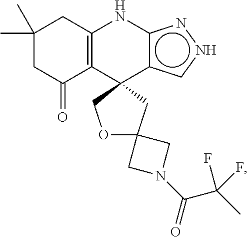 | 947839 |
| 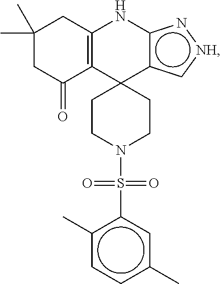 | 947471 |
| 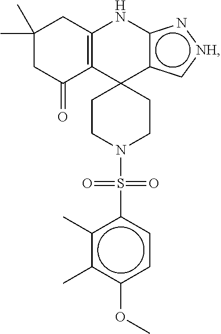 | 945662 |
| 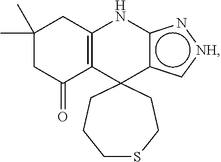 | 913126 |
| 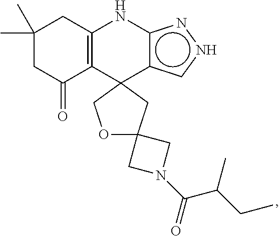 | 947270 |
| 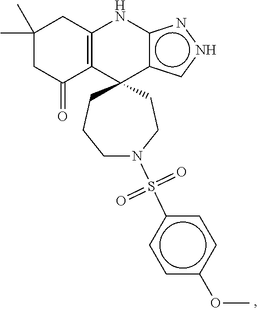 | 932641 |
| 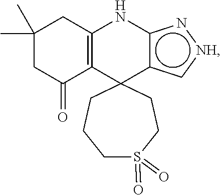 | 927343 |
| 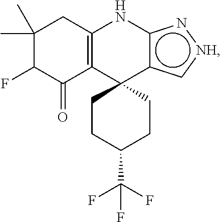 | 917134 |
| 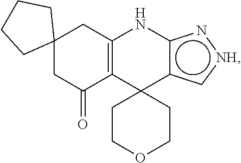 | 893308 |
| 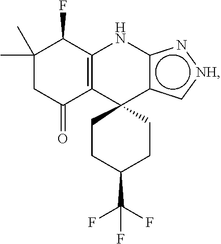 | 918284 |
| 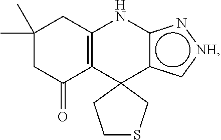 | 883973 |
| 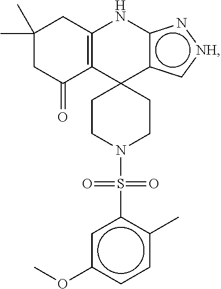 | 947245 |
| 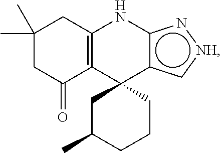 | 874532 |
| 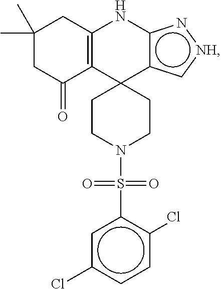 | 948122 |
| 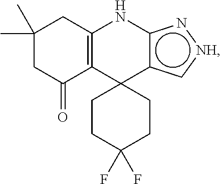 | 883972 |
| 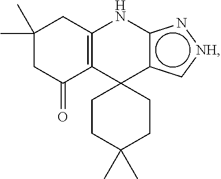 | 884107 |
| 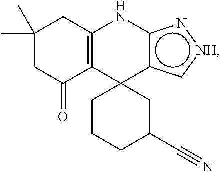 | 923675 |
| 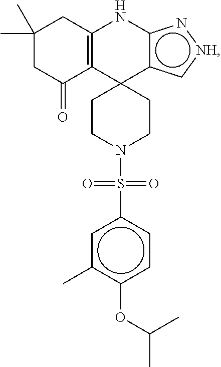 | 945678 |
| 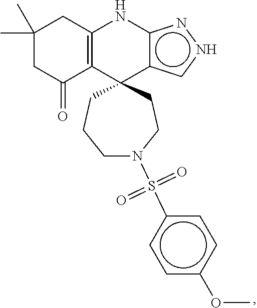 | 932661 |
| 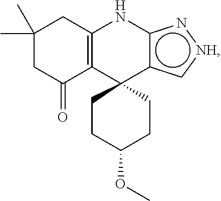 | 884119 |
| 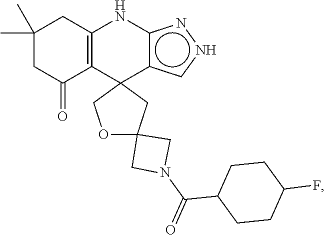 | 947267 |
| 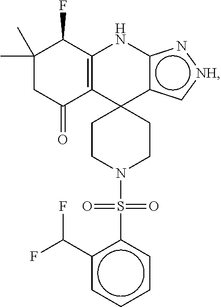 | 951846 |
| 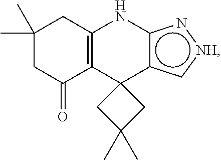 | 883864 |
| 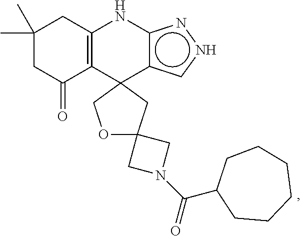 | 947308 |
| 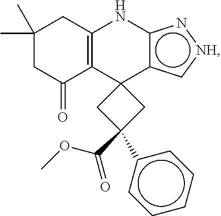 | 942344 |
| 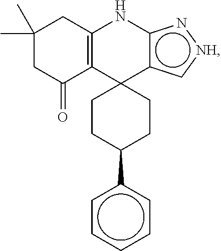 | 874975 |
| 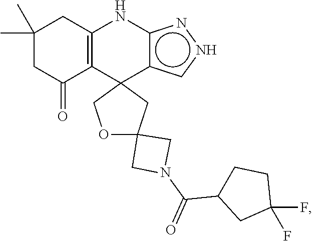 | 947265 |
| 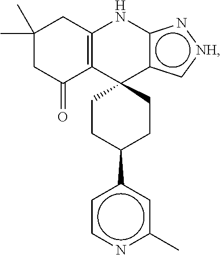 | 948416 |
| 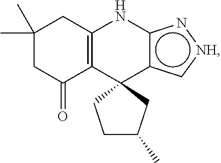 | 897750 |
| 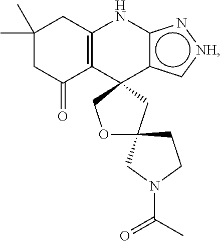 | 948319 |
| 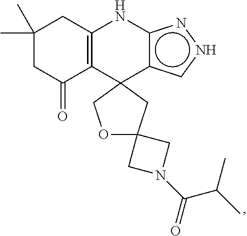 | 947304 |
| 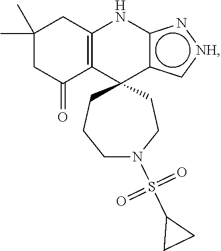 | 932639 |
| 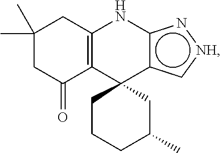 | 893320 |
| 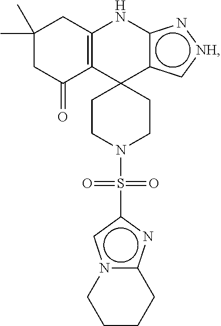 | 947472 |
| 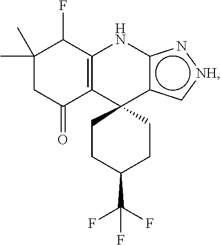 | 917135 |
| 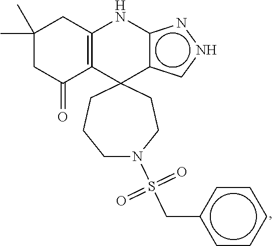 | 932632 |
| 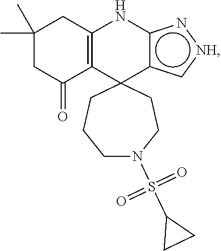 | 917137 |
| 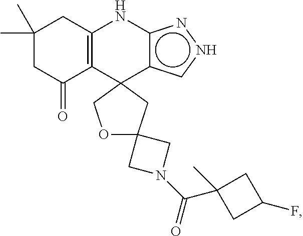 | 947271 |
| 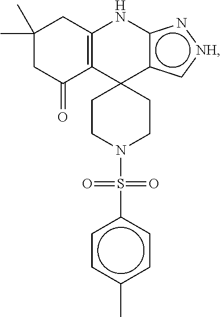 | 947459 |
| 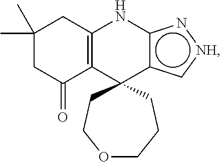 | 874535 |
| 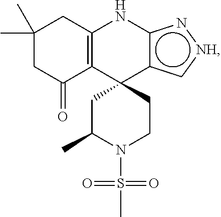 | 926827 |
| 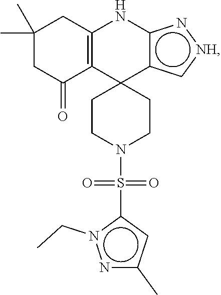 | 947609 |
| 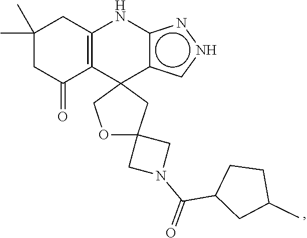 | 947268 |
| 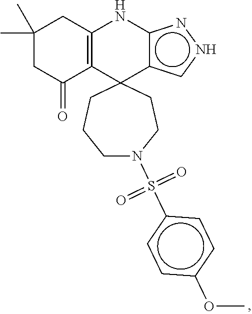 | 917141 |
| 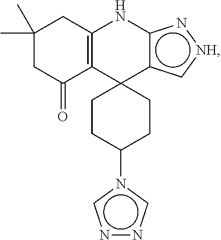 | 943894 |
| 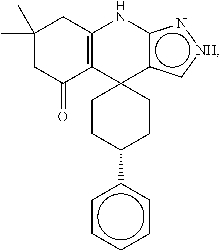 | 912084 |
| 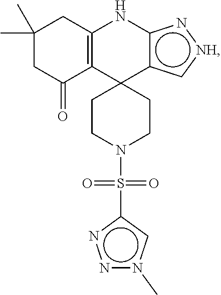 | 947898 |
| 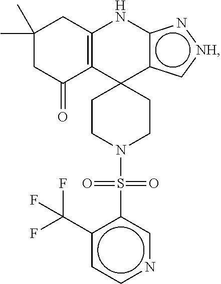 | 947463 |
| 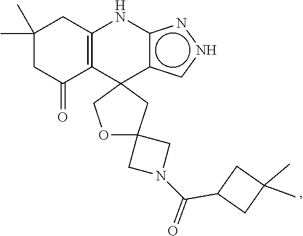 | 947282 |
| 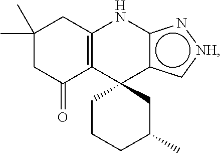 | 893321 |
| 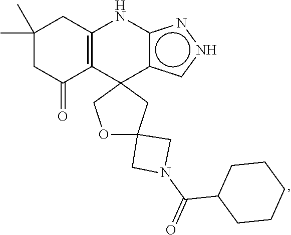 | 947291 |
| 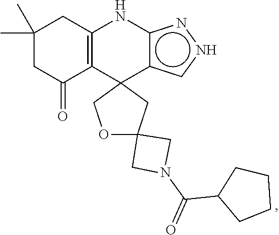 | 917232 |
| 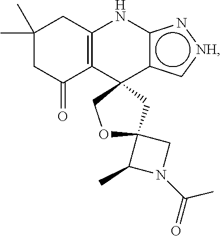 | 948438 |
| 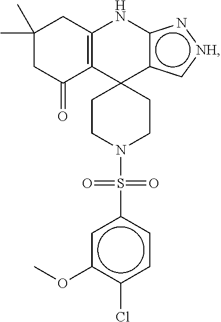 | 945669 |
| 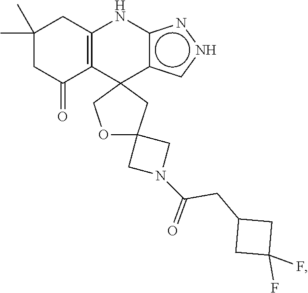 | 947278 |
| 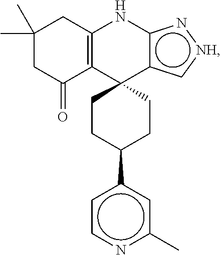 | 948415 |
| 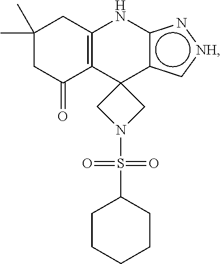 | 918275 |
| 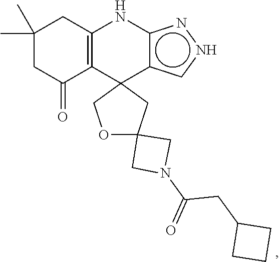 | 947299 |
| 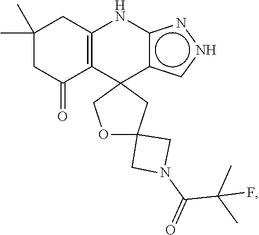 | 947276 |
| 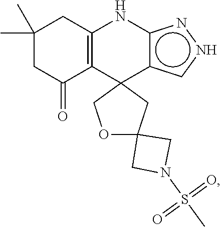 | 913057 |
| 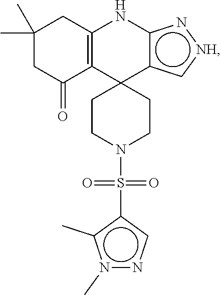 | 947460 |
| 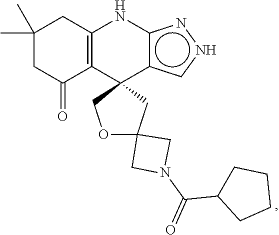 | 943957 |
| 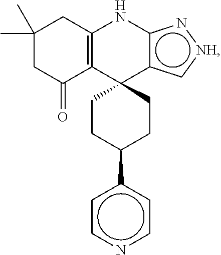 | 949321 |
| 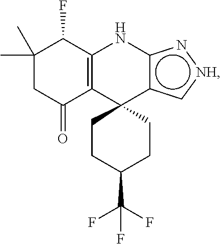 | 918285 |
| 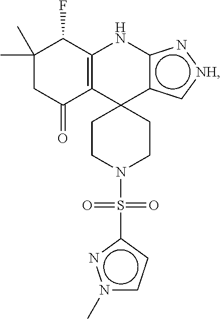 | 951708 |
| 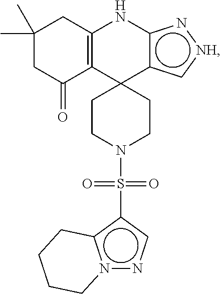 | 947474 |
| 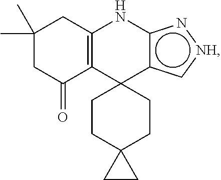 | 893312 |
| 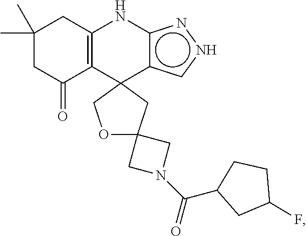 | 947486 |
| 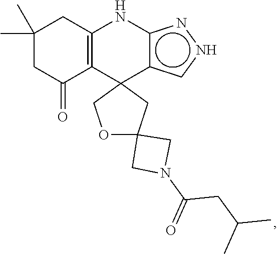 | 947302 |
| 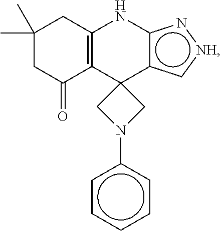 | 948298 |
| 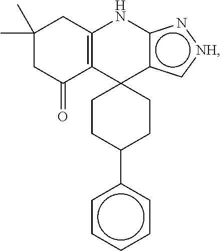 | 877320 |
| 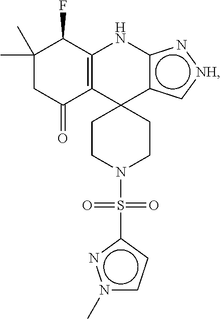 | 951709 |
| 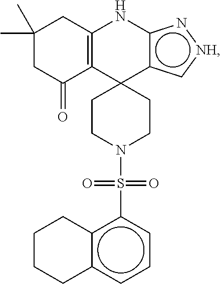 | 947470 |
| 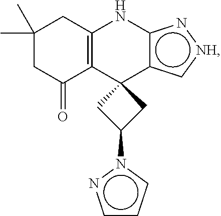 | 884005 |
| 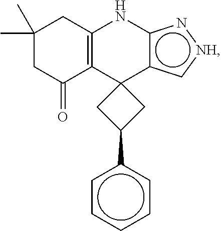 | 917081 |
| 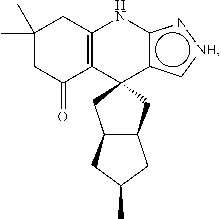 | 943898 |
| 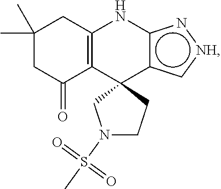 | 942462 |
| 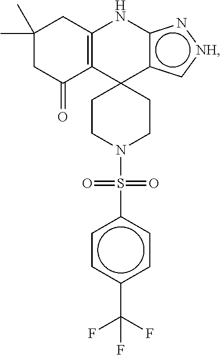 | 947256 |
| 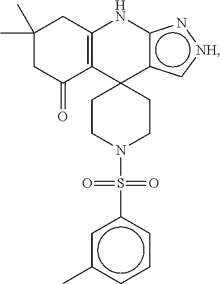 | 947261 |
| 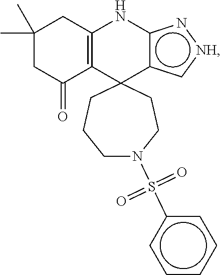 | 889578 |
| 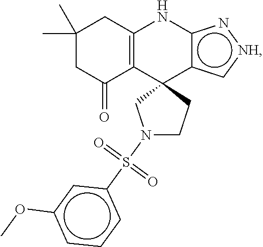 | 951790 |
| 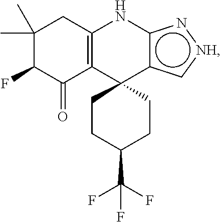 | 918282 |
| 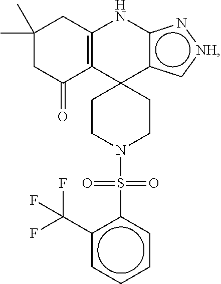 | 945667 |
| 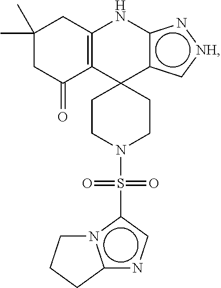 | 947467 |
| 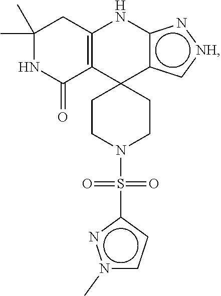 | 943961 |
| 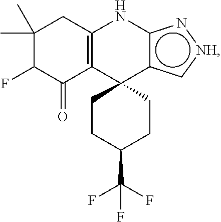 | 917133 |
| 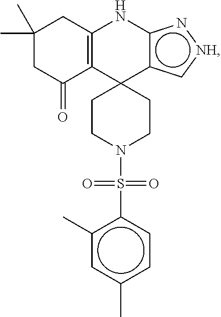 | 947468 |
| 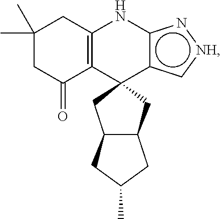 | 943896 |
| 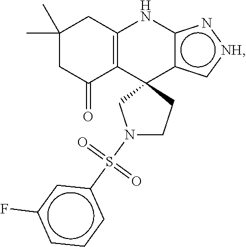 | 951862 |
| 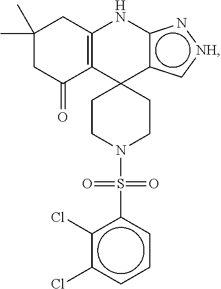 | 947249 |
| 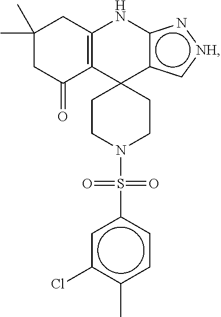 | 947241 |
| 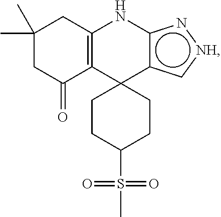 | 913356 |
| 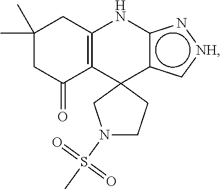 | 912092 |
| 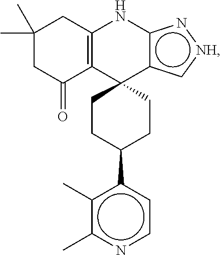 | 950478 |
| 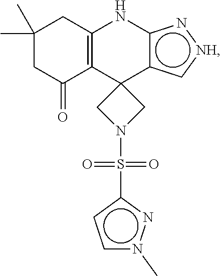 | 913260 |
| 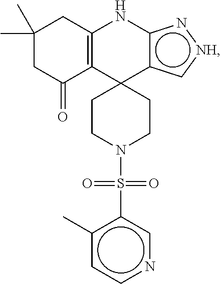 | 945675 |
| 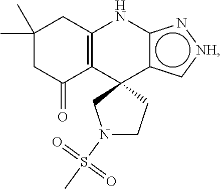 | 942432 |
| 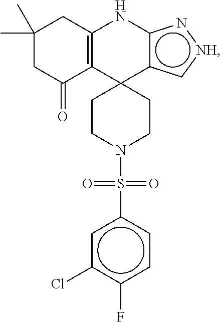 | 948117 |
| 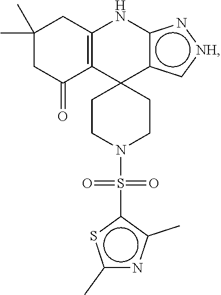 | 947259 |
| 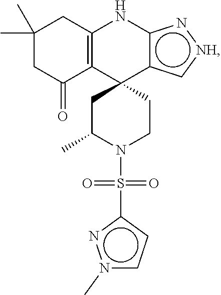 | 917161 |
| 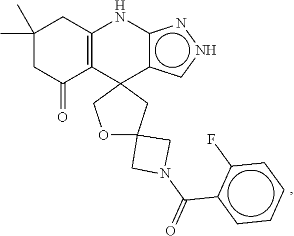 | 947289 |
| 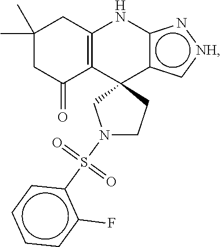 | 945719 |
| 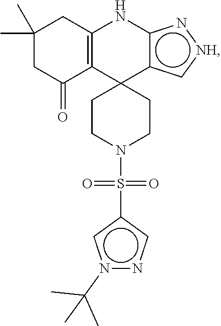 | 945677 |
| 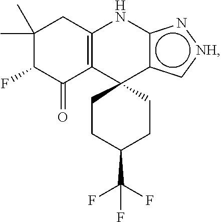 | 918283 |
| 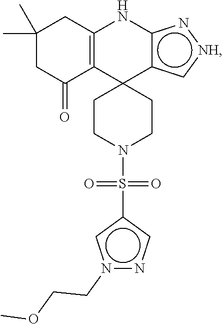 | 947477 |
| 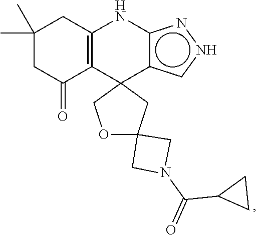 | 947297 |
| 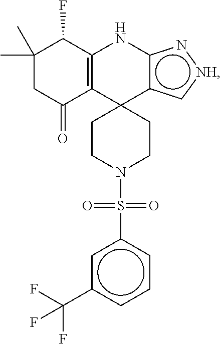 | 951713 |
| 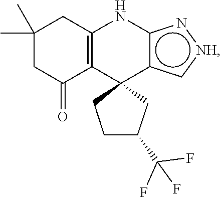 | 952436 |
| 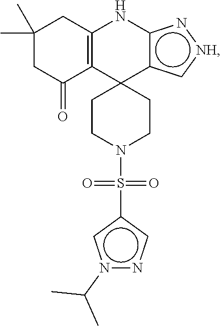 | 947607 |
| 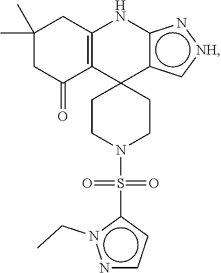 | 945664 |
| 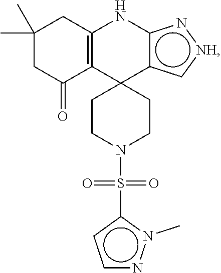 | 932649 |
| 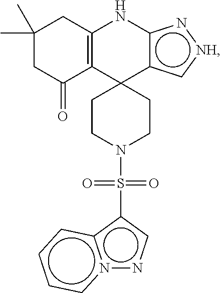 | 947615 |
| 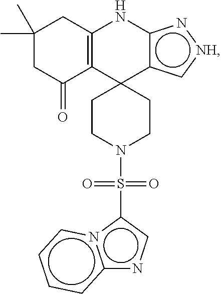 | 947610 |
| 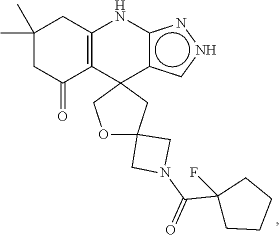 | 947277 |
| 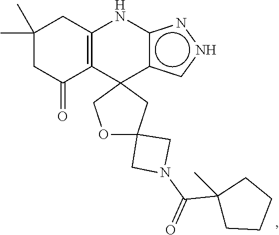 | 947307 |
| 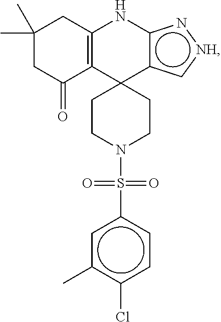 | 947475 |
| 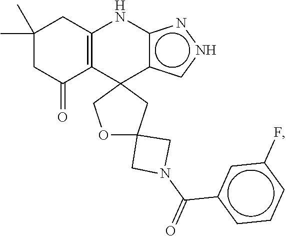 | 947283 |
| 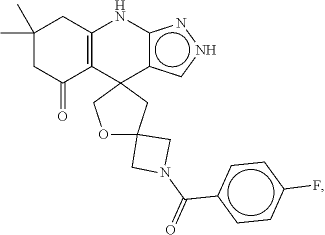 | 947292 |
| 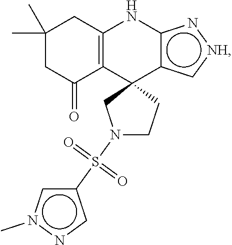 | 943483 |
| 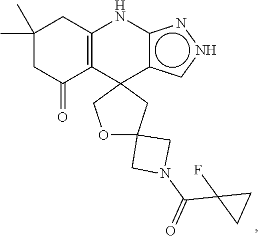 | 947487 |
| 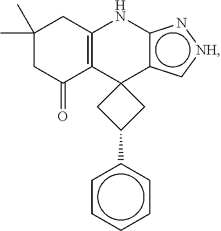 | 917082 |
| 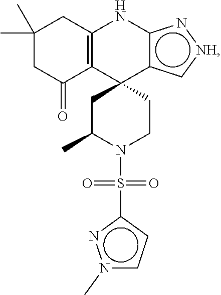 | 926815 |
| 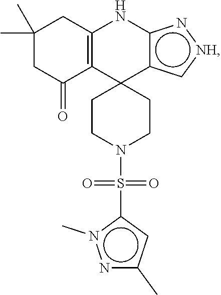 | 947608 |
| 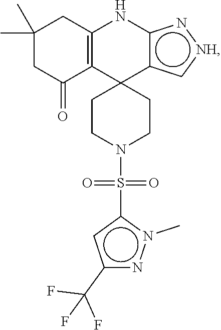 | 947606 |
| 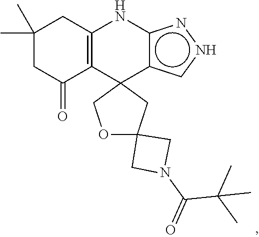 | 947303 |
| 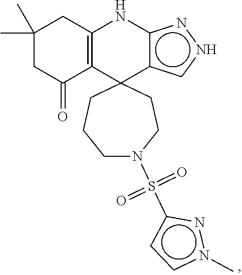 | 917138 |
| 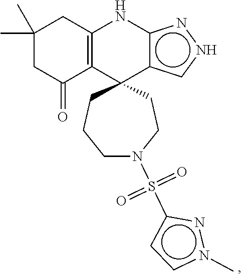 | 932640 |
| 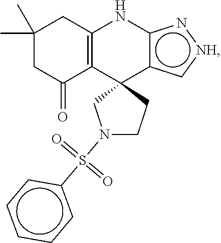 | 951788 |
| 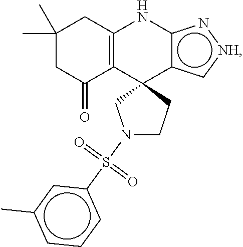 | 951864 |
| 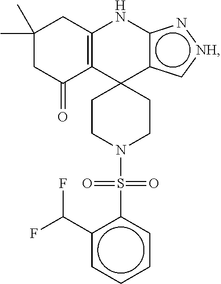 | 945657 |
| 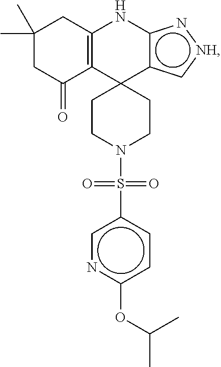 | 945676 |
| 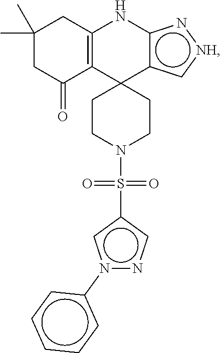 | 947904 |
| 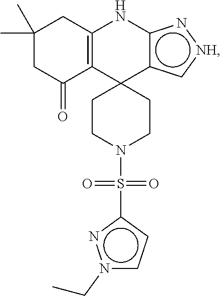 | 945671 |
| 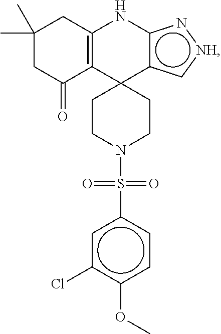 | 947612 |
| 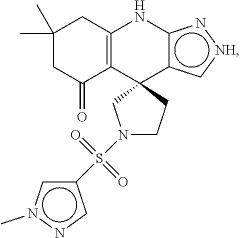 | 951726 |
| 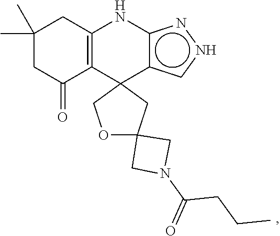 | 947300 |
| 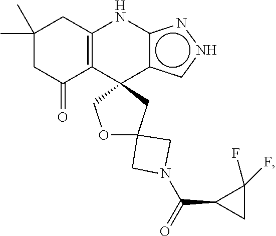 | 948219 |
| 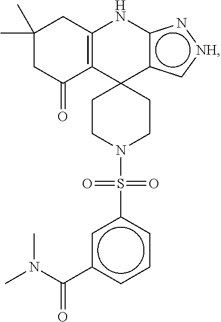 | 947247 |
| 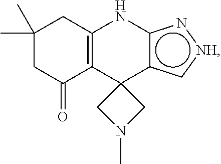 | 913264 |
| 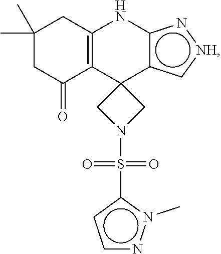 | 932648 |
| 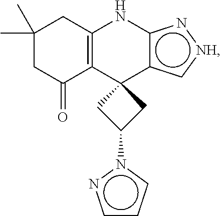 | 884004 |
| 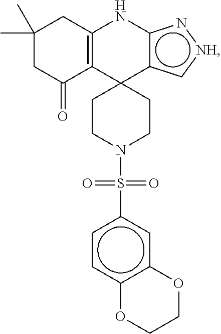 | 947900 |
| 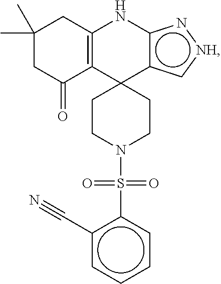 | 947479 |
| 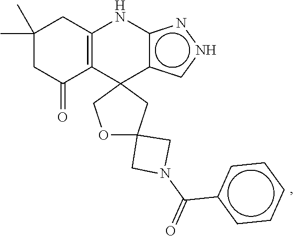 | 917231 |
| 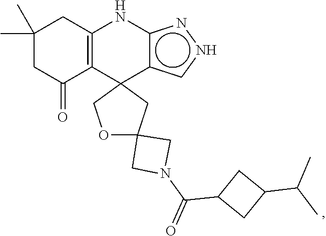 | 947273 |
| 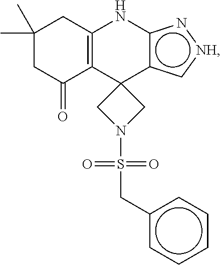 | 943543 |
| 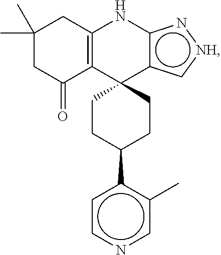 | 949319 |
| 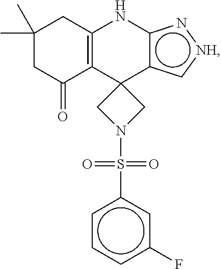 | 918278 |
| 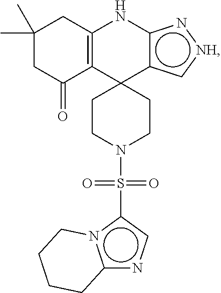 | 947480 |
| 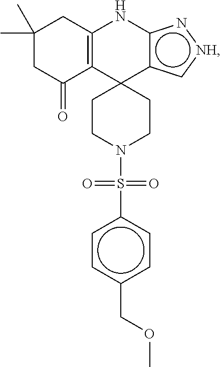 | 947250 |
| 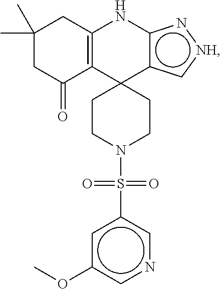 | 947465 |
| 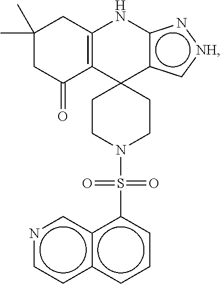 | 947901 |
| 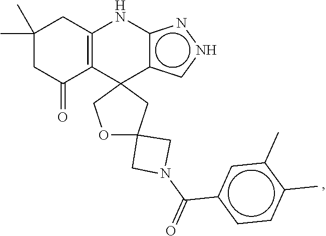 | 947286 |
| 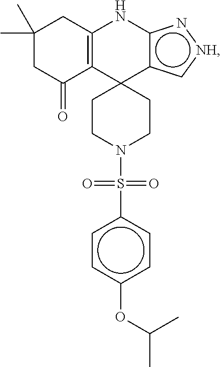 | 945673 |
| 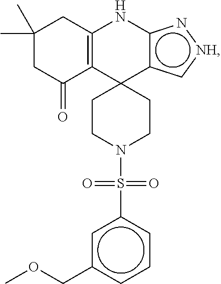 | 947605 |
| 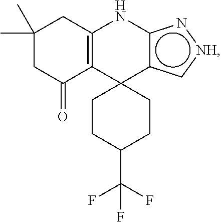 | 865193 |
| 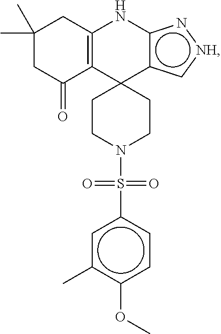 | 945680 |
| 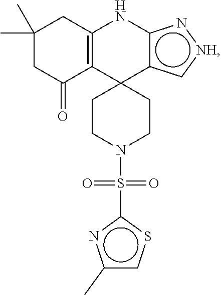 | 947897 |
| 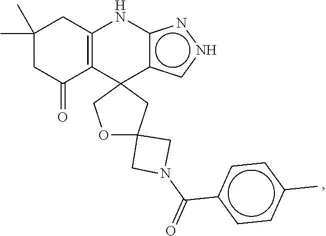 | 947290 |
| 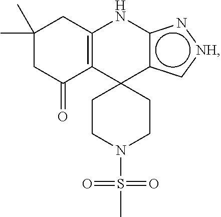 | 880776 |
| 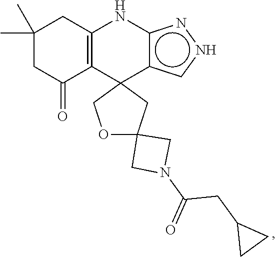 | 947298 |
| 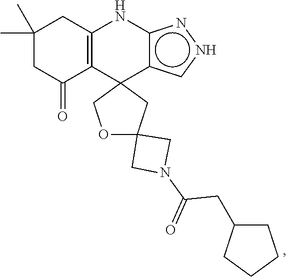 | 947293 |
| 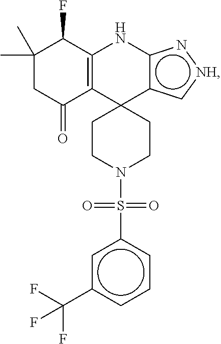 | 951703 |
| 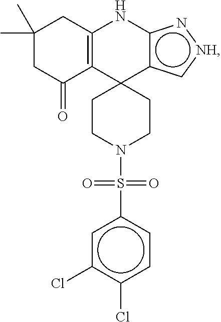 | 945660 |
| 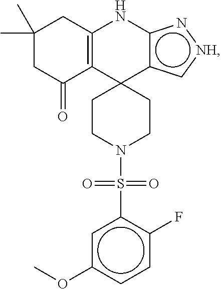 | 945665 |
| 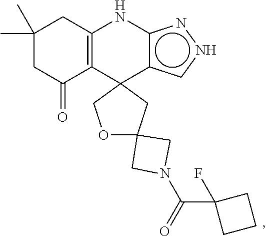 | 947279 |
| 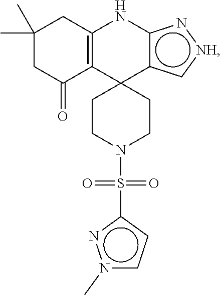 | 901471 |
| 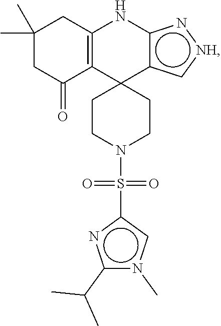 | 945663 |
| 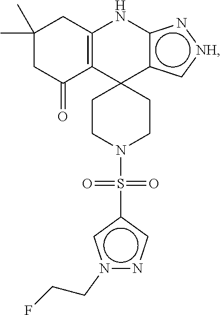 | 947903 |
| 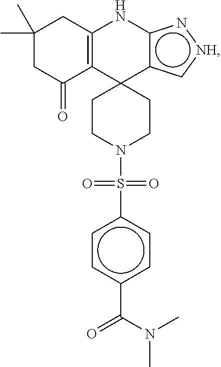 | 945659 |
| 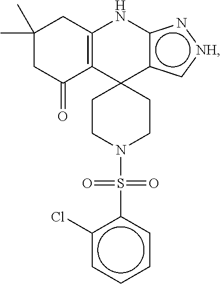 | 945655 |
| 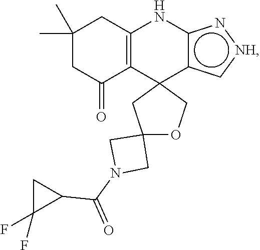 | 947272 |
| 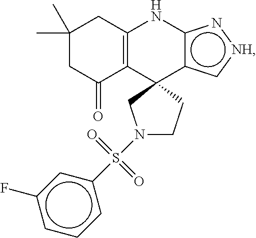 | 943488 |
| 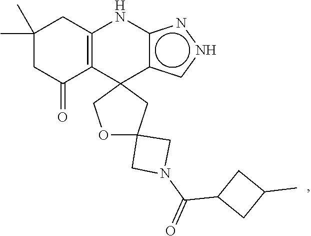 | 947266 |
| 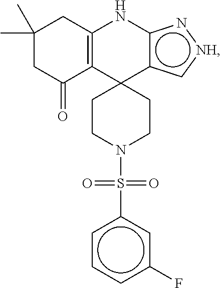 | 901466 |
| 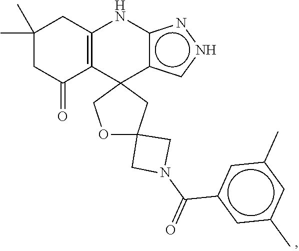 | 947296 |
| 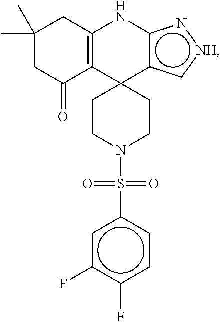 | 948119 |
| 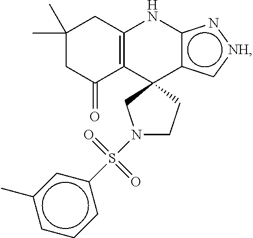 | 943489 |
| 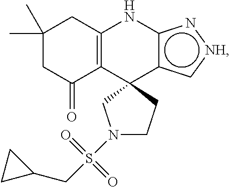 | 951724 |
| 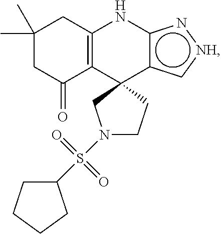 | 943482 |
| 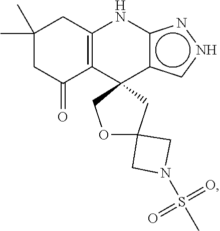 | 926808 |
| 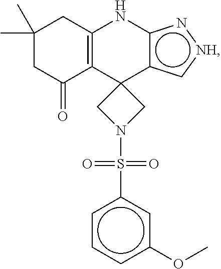 | 918276 |
| 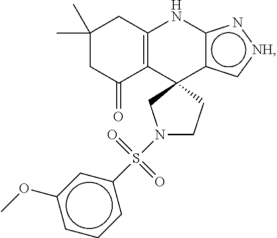 | 943486 |
| 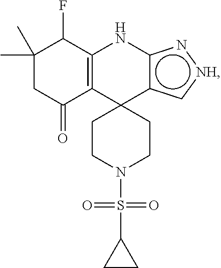 | 950814 |
| 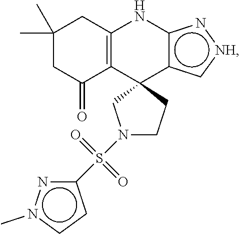 | 945714 |
| 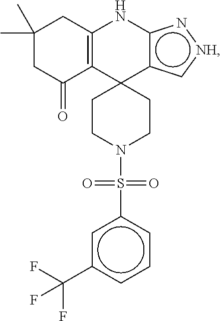 | 947244 |
| 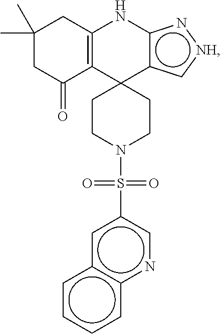 | 947462 |
| 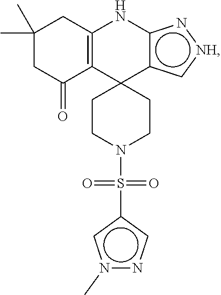 | 901467 |
| 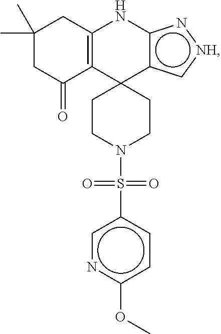 | 945666 |
| 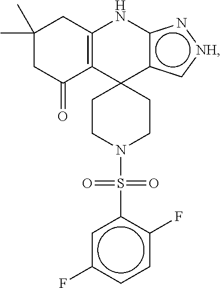 | 948121 |
| 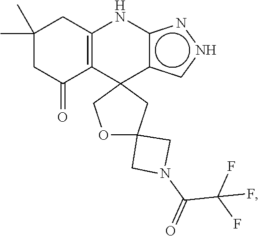 | 947280 |
| 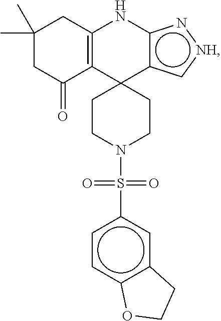 | 913251 |
| 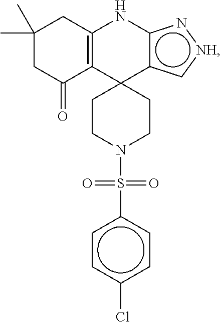 | 945661 |
| 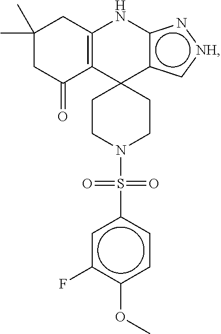 | 947478 |
| 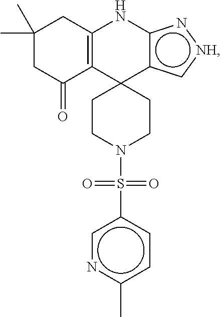 | 947482 |
| 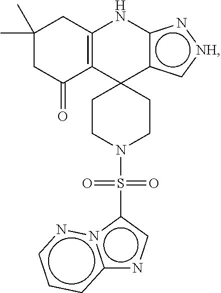 | 948120 |
| 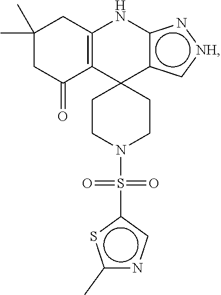 | 947899 |
| 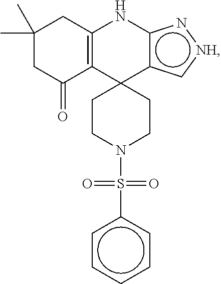 | 874530 |
| 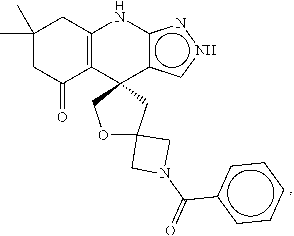 | 943955 |
| 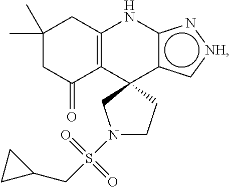 | 943481 |
| 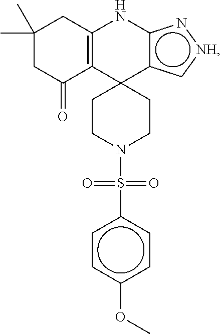 | 917143 |
| 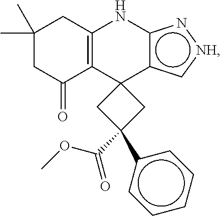 | 942366 |
| 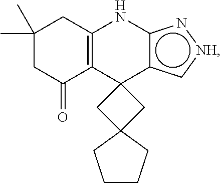 | 874477 |
| 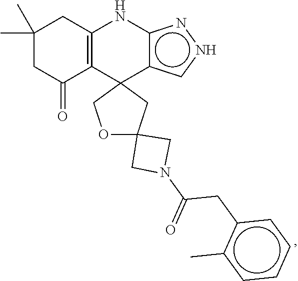 | 947295 |
| 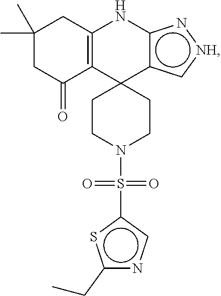 | 947252 |
| 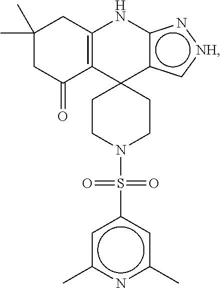 | 947616 |
| 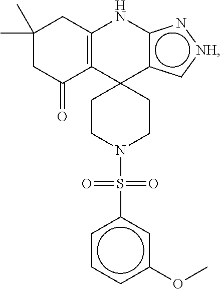 | 901468 |
| 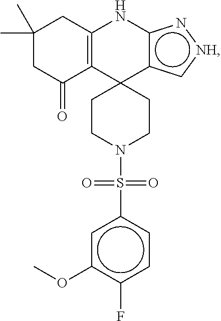 | 947263 |
| 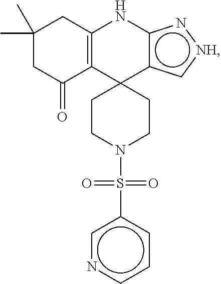 | 947613 |
| 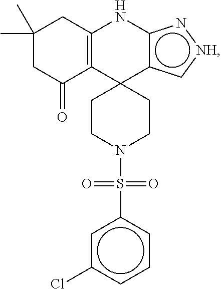 | 947243 |
| 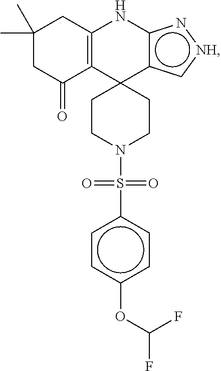 | 947246 |
| 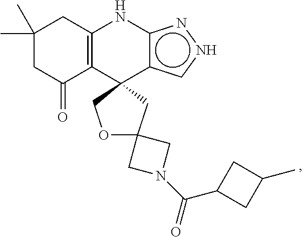 | 947733 |
| 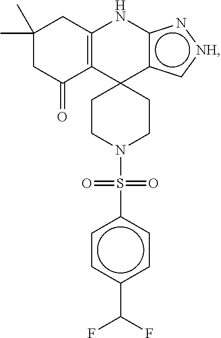 | 947248 |
| 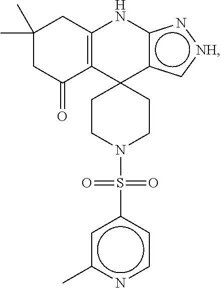 | 947481 |
| 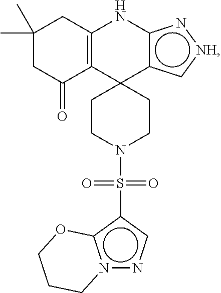 | 947483 |
| 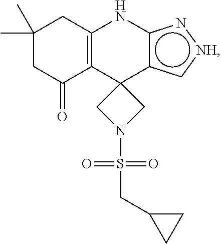 | 929440 |
| 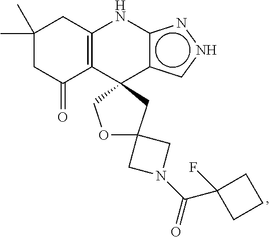 | 947735 |
| 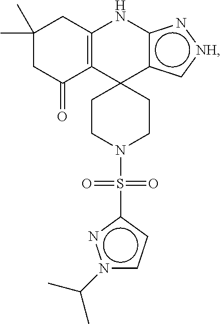 | 947258 |
| 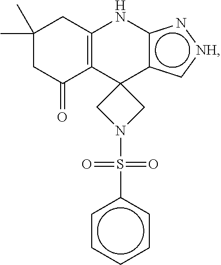 | 901474 |
| 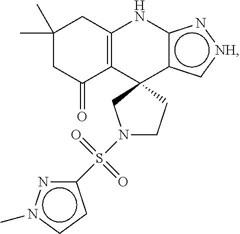 | 943484 |
| 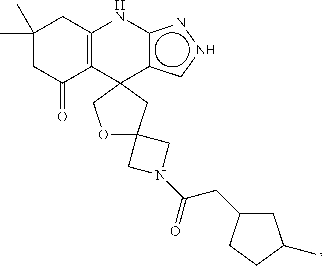 | 947269 |
| 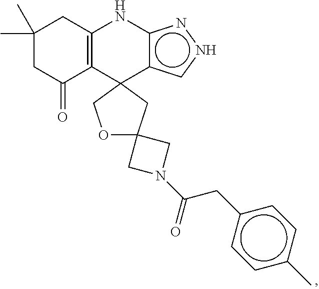 | 947281 |
| 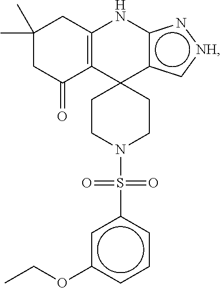 | 947253 |
| 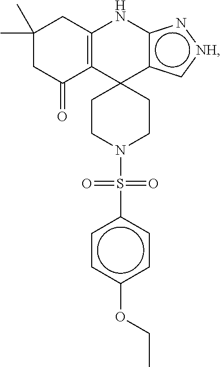 | 945670 |
| 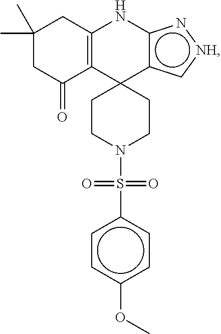 | 901469 |
| 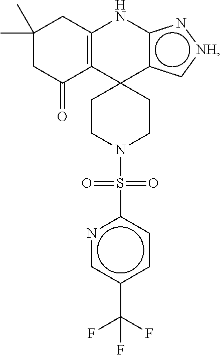 | 947902 |
| 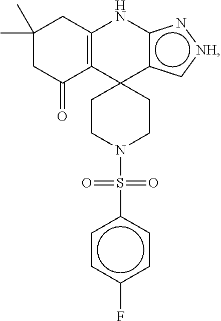 | 893311 |
| 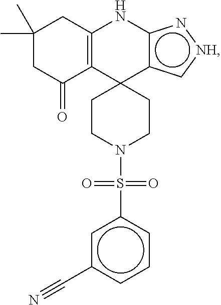 | 947254 |
| 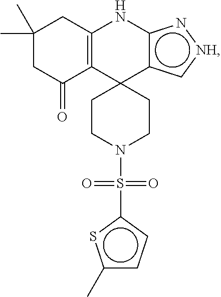 | 945672 |
| 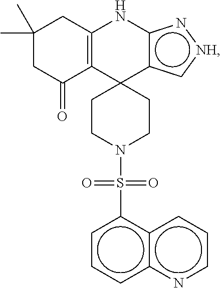 | 947469 |
| 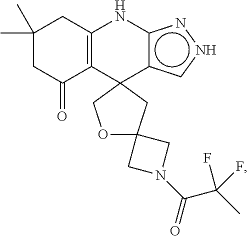 | 947275 |
| 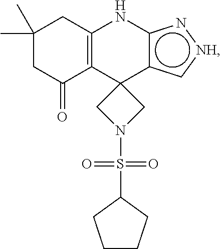 | 917139 |
| 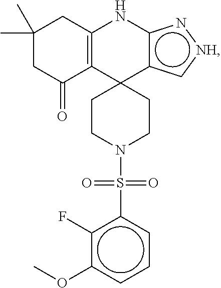 | 945656 |
| 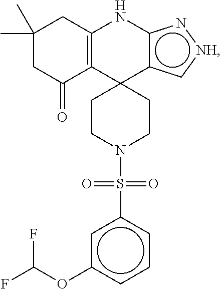 | 947242 |
| 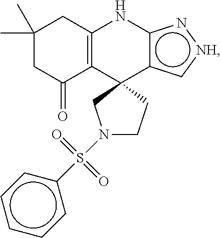 | 943485 |
| 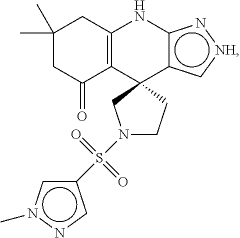 | 951725 |
| 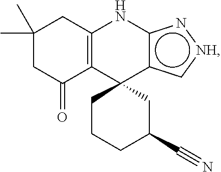 | 945805 |
| 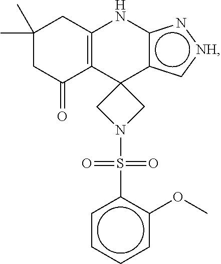 | 913261 |
| 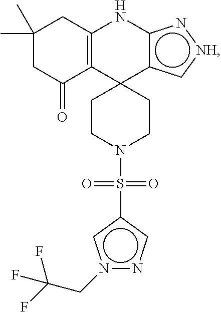 | 948123 |
| 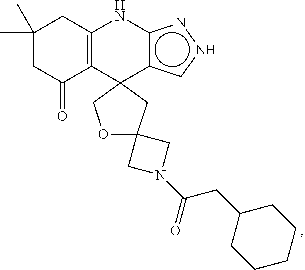 | 947305 |
| 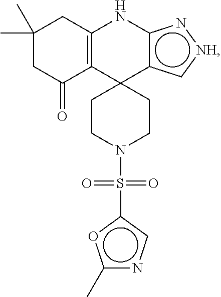 | 947611 |
| 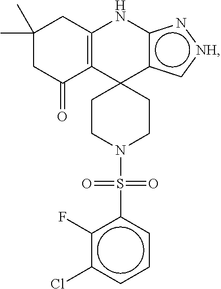 | 948118 |
| 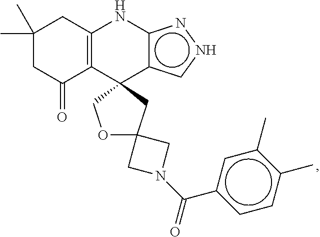 | 948038 |
| 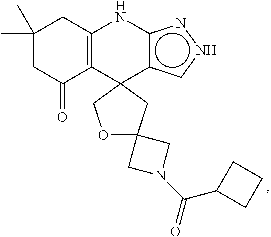 | 947288 |
| 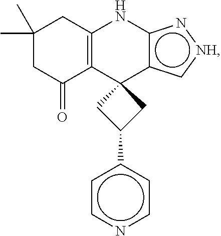 | 889665 |
| 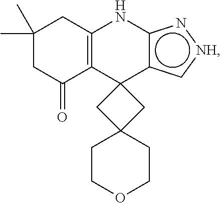 | 874534 |
| 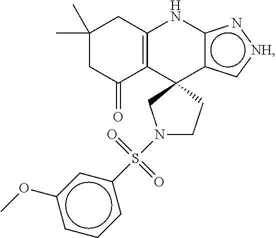 | 951789 |
| 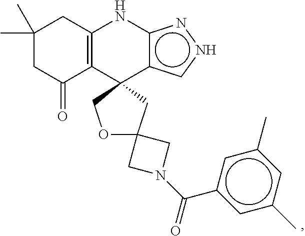 | 948223 |
| 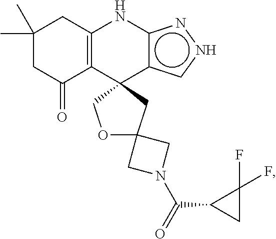 | 948215 |
| 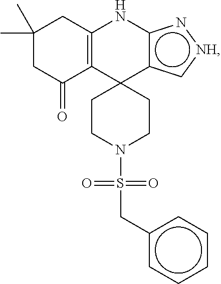 | 929437 |
| 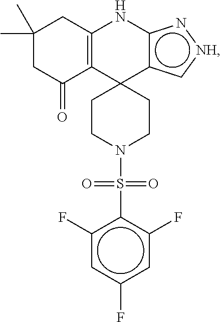 | 947484 |
| 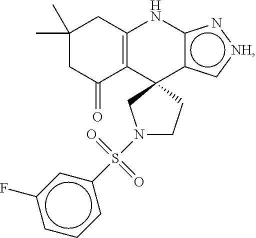 | 951861 |
| 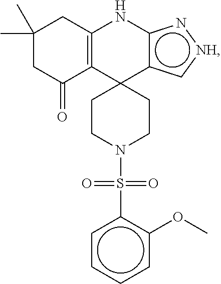 | 893310 |
| 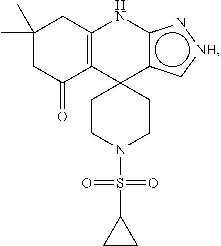 | 901470 |
| 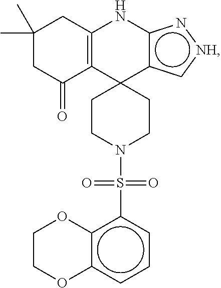 | 947464 |
| 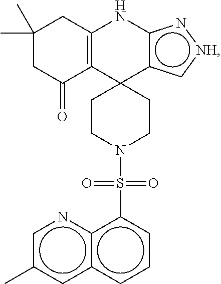 | 947614 |
| 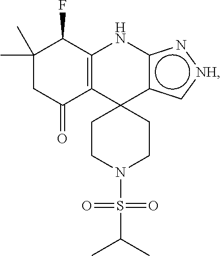 | 951701 |
| 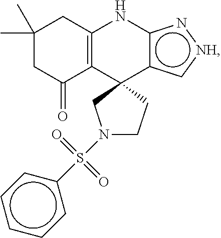 | 951787 |
| 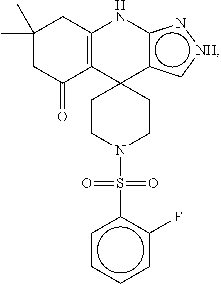 | 901465 |
| 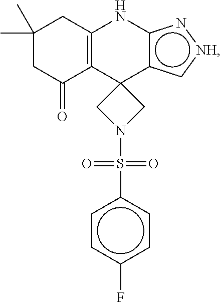 | 917144 |
| 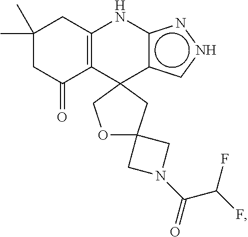 | 947485 |
| 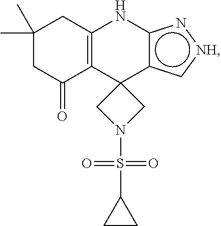 | 913259 |
| 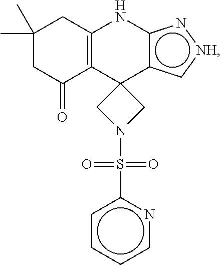 | 913262 |
| 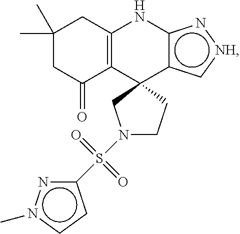 | 945712 |
| 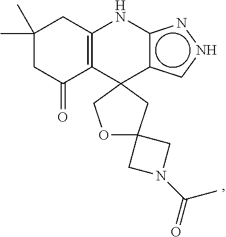 | 913058 |
| 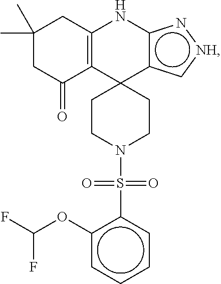 | 945668 |
| 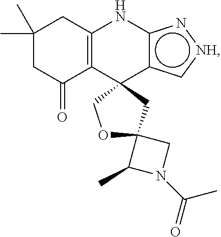 | 948440 |
| 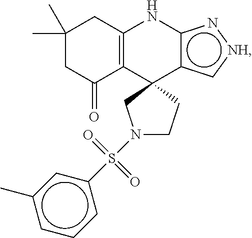 | 951863 |
| 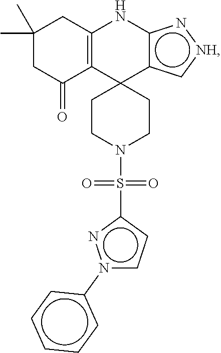 | 947461 |
| 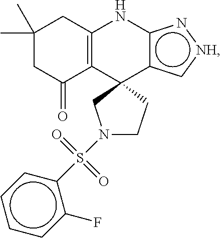 | 943487 |
| 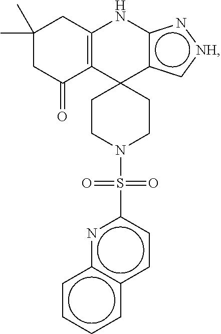 | 947476 |
| 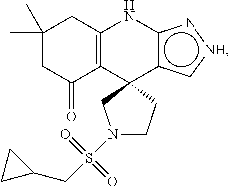 | 951723 |
| 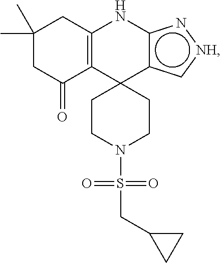 | 909207 |
| 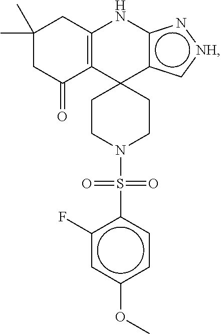 | 947255 |
| 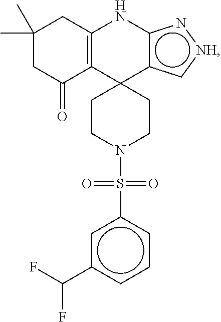 | 947264 |
| 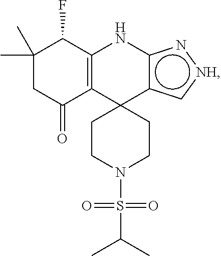 | 951714 |
| 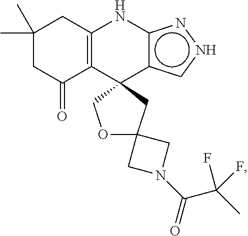 | 947838 |
| 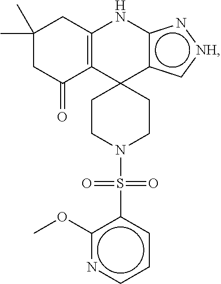 | 947260 |
| 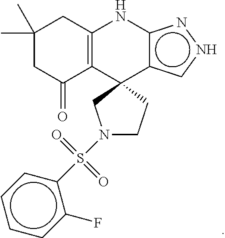 | 945718 |

79. The compound of claim 58, or a pharmaceutically acceptable salt, solvate, hydrate, thereof, wherein the compound is any one of the formulae shown in Table 5A:
TABLE 5A

| | |
|---|---|
| 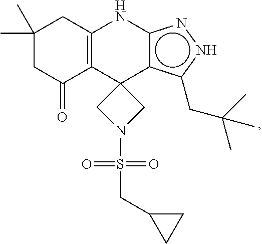 | 90285 |
| 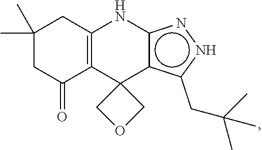 | 90709 |
| 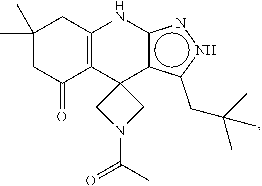 | 90991 |
| 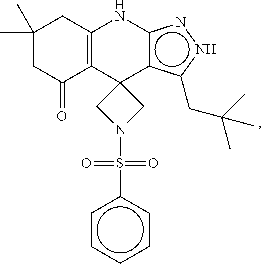 | 92737 |
| 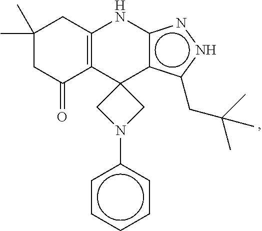 | 93429 |
| 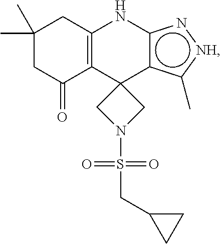 | 107828 |
| 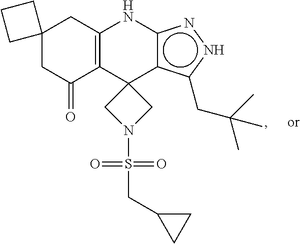 | 107829 |
| 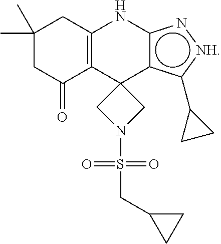 | 109152 |

80. The compound of claim 58, or a pharmaceutically acceptable salt, solvate, hydrate, polymorph, co-crystal, tautomer, stereoisomer, isotopically labeled compound, or prodrug thereof, wherein the compound is any one of the formulae shown in Table 6:
TABLE 6

| | |
|---|---|
| 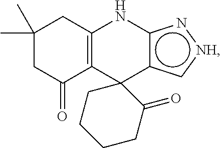 | 912105 |
| 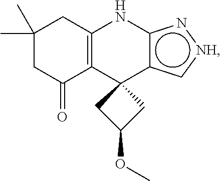 | 901472 |
| 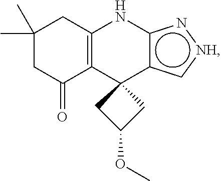 | 901473 |
| 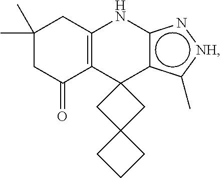 | 877295 |
| 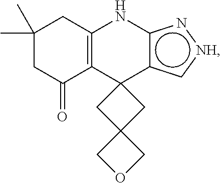 | 912168 |
| 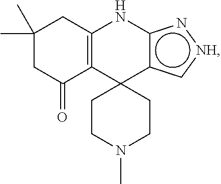 | 826714 |
| 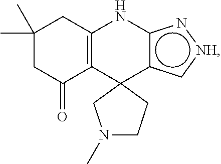 | 841867 |
| 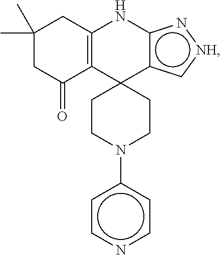 | 880765 |
| 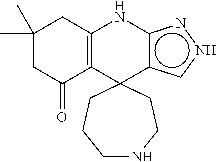 | 889571 |
| 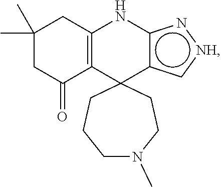 | 893313 |
| 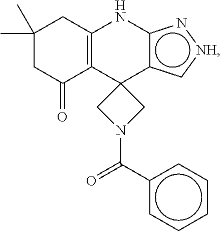 | 893315 |
| 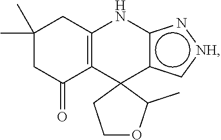 | 911958 |
| 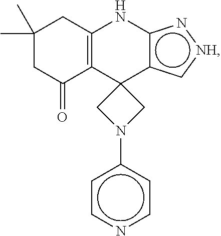 | 912077 |
| 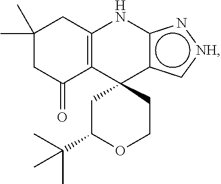 | 912107 |
| 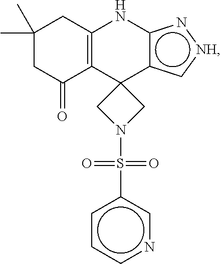 | 913265 |
| 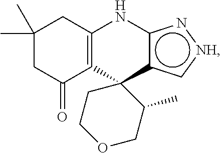 | 924310 |
| 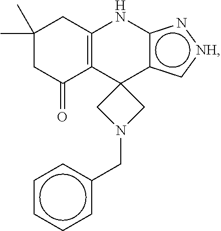 | 929436 |
| 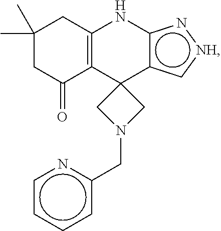 | 929438 |
| 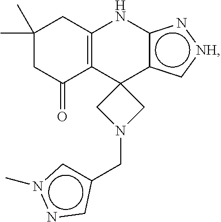 | 929439 |
| 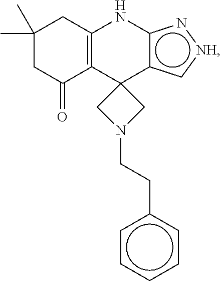 | 929441 |
| 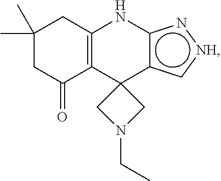 | 929448 |
| 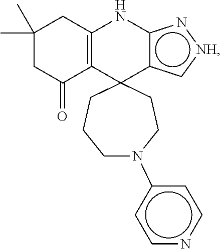 | 932633 |
| 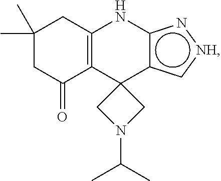 | 932650 |
| 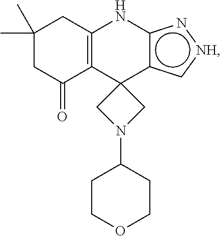 | 932651 |
| 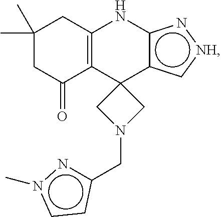 | 932652 |
| 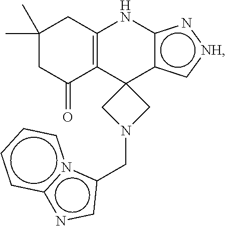 | 932654 |
| 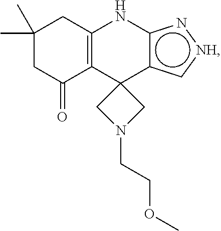 | 943544 |
| 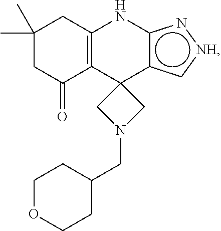 | 943545 |
| 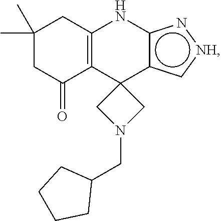 | 943914 |

81. The compound of claim 58, or a pharmaceutically acceptable salt, solvate, hydrate, polymorph, co-crystal, tautomer, stereoisomer, isotopically labeled compound, or prodrug thereof, wherein the compound is any one of the formulae shown in Table 6A:
TABLE 6A

| 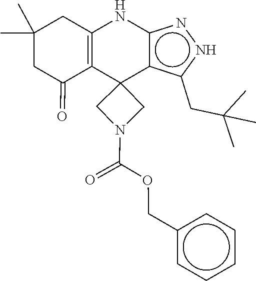 | 90330 |
|---|---|
| or | |
| 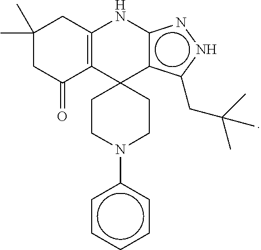 | 92315 |

82. The compound of claim 58, or a pharmaceutically acceptable salt, solvate, hydrate, polymorph, co-crystal, tautomer, stereoisomer, isotopically labeled compound, or prodrug thereof, wherein the compound is not a compound of claim 80 or 81.

83. A compound, or a pharmaceutically acceptable salt, solvate, hydrate, polymorph, co-crystal, tautomer, stereoisomer, isotopically labeled compound, or prodrug thereof, wherein the compound is of Formula IV-A or IV-B: 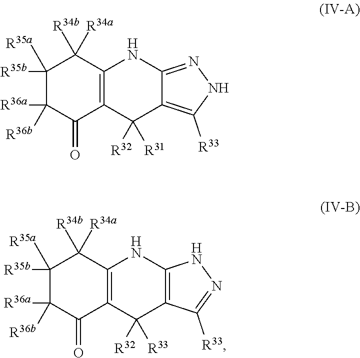
wherein:
R³¹ is 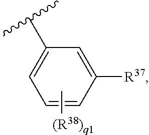
optionally substituted naphthyl, optionally substituted furanyl, optionally substituted thiophenyl, optionally substituted pyrrolyl, optionally substituted imidazolyl, optionally substituted pyrazolyl, optionally substituted oxazolyl, optionally substituted isoxazolyl, optionally substituted thiazolyl, optionally substituted isothiazolyl, optionally substituted triazolyl, optionally substituted oxadiazolyl, optionally substituted thiadiazolyl, optionally substituted tetrazolyl, optionally substituted pyridinyl, optionally optionally substituted triazinyl, optionally substituted tetrazinyl, optionally substituted bicyclic heteroaryl, optionally substituted carbocyclyl, or optionally substituted heterocyclyl;
R³² is optionally substituted alkyl, optionally substituted alkenyl, or optionally substituted alkynyl;
each of R³³ and R³⁸ is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(R^{D})₃, —Osi(R^{D})₂OR^{D}, —Osi(R^{D})(OR^{D})₂, or —Osi(OR^{D})₃;
each of R^{34a}, R^{34b}, R^{35a}, R^{35b}, R^{36a}, and R^{36b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each instance of R^{D} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{D} attached to the same intervening atom are joined together with the intervening atom to form an optionally substituted, monocyclic, heterocyclic or heteroaryl ring;
or R^{34a} and R^{34b} are taken together with their intervening atom to form an optionally substituted, monocyclic, carbocyclic or heterocyclic ring;
or R^{34b} and R^{35a} are taken together with their intervening atom to form an optionally substituted, monocyclic, carbocyclic or heterocyclic ring;
or R^{35a} and R^{35b} are taken together with their intervening atom to form an optionally substituted, monocyclic, carbocyclic or heterocyclic ring;
or R^{35b} and R^{36a} are taken together with their intervening atom to form an optionally substituted, monocyclic, carbocyclic or heterocyclic ring; and
or R^{36a} and R^{36b} are taken together with their intervening atom to form an optionally substituted, monocyclic, carbocyclic or heterocyclic ring;
q1 is 0, 1, 2, 3, or 4; and
R³⁷ is —Br, —I, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(R^{D}) s, —OSi(R^{D})₂OR^{D}, —OSi(R^{D})(OR^{D})₂, or —OSi(OR^{D})₃;
provided that R³⁷ is not-C(CH₃)₃, —CF₃, —CN, or —C(═O) NH₂; and
provided that each of Formulae IV-A and IV-B is not

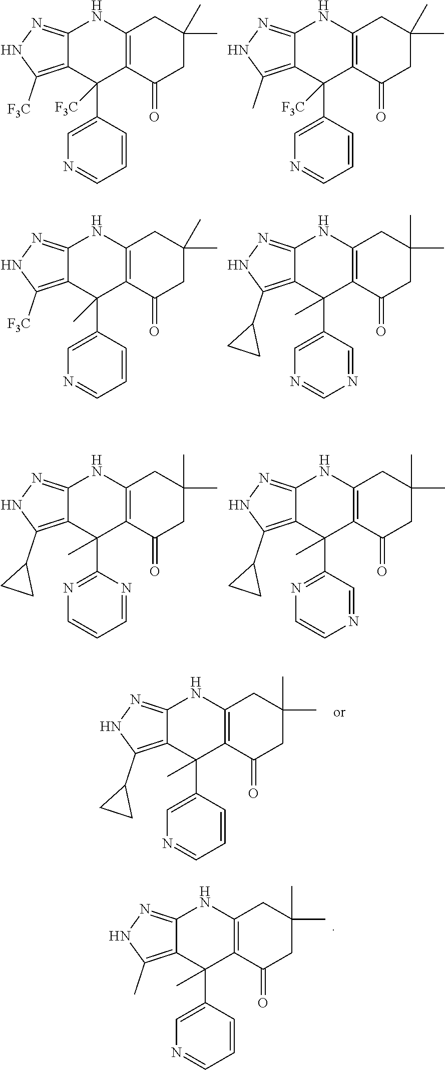

84. The compound of claim 83, or a pharmaceutically acceptable salt, solvate, hydrate, polymorph, co-crystal, tautomer, stereoisomer, isotopically labeled compound, or prodrug thereof, wherein each of R^{36a} and R^{36b} is independently hydrogen, halogen, or optionally substituted alkyl.

85. The compound of claim 84, or a pharmaceutically acceptable salt, solvate, hydrate, polymorph, co-crystal, tautomer, stereoisomer, isotopically labeled compound, or prodrug thereof, wherein at least one of R^{36a} and R^{36b} is fluorine.

86. The compound of claim 84, or a pharmaceutically acceptable salt, solvate, hydrate, polymorph, co-crystal, tautomer, stereoisomer, isotopically labeled compound, or prodrug thereof, wherein R^{36a} and R^{36b} are hydrogen.

87. The compound of any one of claims 83-86, or a pharmaceutically acceptable salt, solvate, hydrate, polymorph, co-crystal, tautomer, stereoisomer, isotopically labeled compound, or prodrug thereof, wherein each of R^{35a} and R^{35b} is independently hydrogen, halogen, or optionally substituted alkyl.

88. The compound of claim 87, or a pharmaceutically acceptable salt, solvate, hydrate, polymorph, co-crystal, tautomer, stereoisomer, isotopically labeled compound, or prodrug thereof, wherein R^{35a} and R^{35b} are —CH₃.

89. The compound of any one of claims 83-86, or a pharmaceutically acceptable salt, solvate, hydrate, polymorph, co-crystal, tautomer, stereoisomer, isotopically labeled compound, or prodrug thereof, wherein R^{35a} and R^{35b} are taken together with their intervening atom to form an optionally substituted, monocyclic, carbocyclic or heterocyclic ring.

90. The compound of any one of claims 83-89, or a pharmaceutically acceptable salt, solvate, prodrug thereof, wherein each of R^{34a} and R^{34b} is independently hydrogen, halogen, or optionally substituted alkyl.

91. The compound of claim 90, or a pharmaceutically acceptable salt, solvate, hydrate, polymorph, co-crystal, tautomer, stereoisomer, isotopically labeled compound, or prodrug thereof, wherein at least one of R^{34a} and R^{34b} is fluorine.

92. The compound of claim 90, or a pharmaceutically acceptable salt, solvate, hydrate, polymorph, co-crystal, tautomer, stereoisomer, isotopically labeled compound, or prodrug thereof, wherein R^{34a} and R^{34b} are hydrogen.

93. The compound of any one of claims 83-92, or a pharmaceutically acceptable salt, solvate, hydrate, polymorph, co-crystal, tautomer, stereoisomer, isotopically labeled compound, or prodrug thereof, wherein R³¹ is

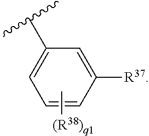

94. The compound of any one of claims 83-93, or a pharmaceutically acceptable salt, solvate, hydrate, polymorph, co-crystal, tautomer, stereoisomer, isotopically labeled compound, or prodrug thereof, wherein R³⁷ is —Br, optionally substituted alkyl, optionally substituted heteroalkyl, optionally substituted carbocyclyl, optionally substituted heterocyclyl, optionally substituted aryl, optionally substituted heteroaryl, —OR^{D}, —SR^{D}, —N(R^{D})₂, —S(═O)₂R^{D}, —NR^{D}C(═O)R^{D}, or —NR^{D}S(═O)₂SR^{D}.

95. The compound of claim 94, or a pharmaceutically acceptable salt, solvate, hydrate, polymorph, co-crystal, tautomer, stereoisomer, isotopically labeled compound, or prodrug thereof, wherein R³⁷ is optionally substituted heteroaryl comprising one or more N atoms.

96. The compound of claim 95, or a pharmaceutically acceptable salt, solvate, hydrate, polymorph, co-crystal, tautomer, stereoisomer, isotopically labeled compound, or prodrug thereof, wherein R³⁷ is optionally substituted pyridinyl or optionally substituted pyrazolyl.

97. The compound of claim 94, or a pharmaceutically acceptable salt, solvate, hydrate, thereof, wherein R³⁷ is optionally substituted heterocyclyl.

98. The compound of any one of claims 83-97, or a pharmaceutically acceptable salt, solvate, hydrate, polymorph, co-crystal, tautomer, stereoisomer, isotopically labeled compound, or prodrug thereof, wherein q1 is 0.

99. The compound of any one of claims 83-98, or a pharmaceutically acceptable salt, solvate, hydrate, polymorph, co-crystal, tautomer, stereoisomer, isotopically labeled compound, or prodrug thereof, wherein R³² is optionally substituted alkyl.

100. The compound of claim 99, or a pharmaceutically acceptable salt, solvate, hydrate, polymorph, co-crystal, tautomer, stereoisomer, isotopically labeled compound, or prodrug thereof, wherein R³² is unsubstituted C₁-C₄ alkyl.

101. The compound of any one of claims 83-100, or a pharmaceutically acceptable salt, solvate, hydrate, polymorph, co-crystal, tautomer, stereoisomer, isotopically labeled compound, or prodrug thereof, wherein R³³ is hydrogen, optionally substituted C₁-C₆ alkyl, or halogen.

102. The compound of claim 101, or a pharmaceutically acceptable salt, solvate, hydrate, polymorph, co-crystal, tautomer, stereoisomer, isotopically labeled compound, or prodrug thereof, wherein R³³ is hydrogen, fluorine, —CH₃, —CH₂F, —CHF₂, or —CF₃.

103. The compound of claim 101, or a pharmaceutically acceptable salt, solvate, hydrate, polymorph, co-crystal, tautomer, stereoisomer, isotopically labeled compound, or prodrug thereof, wherein R³³ is C₁₋₄ haloalkyl.

104. The compound of claim 83, or a pharmaceutically acceptable salt, solvate, hydrate, polymorph, co-crystal, tautomer, stereoisomer, isotopically labeled compound, or prodrug thereof, wherein the compound is of the formula;

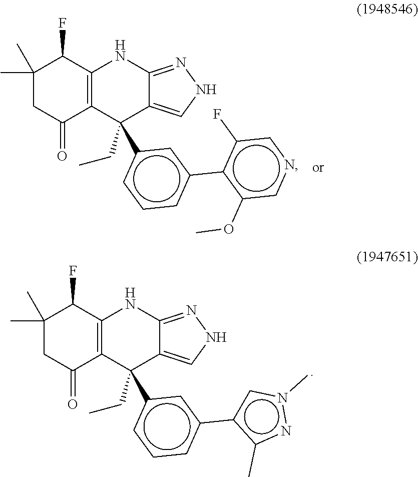

105. The compound of claim 83, or a pharmaceutically acceptable salt, solvate, hydrate, polymorph, co-crystal, tautomer, stereoisomer, isotopically labeled compound, or prodrug thereof, wherein the compound is any one of the formulae shown in Table 7:
TABLE 7

| | |
|---|---|
| 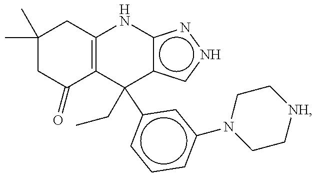 | 806604 |
| 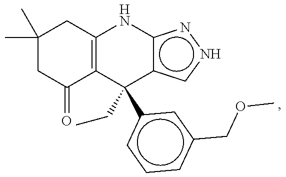 | 810362 |
| 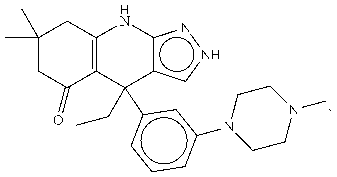 | 810379 |
| 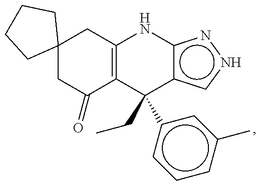 | 801313 |
| 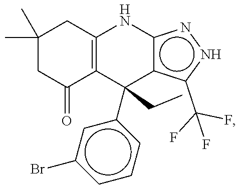 | 810365 |
| 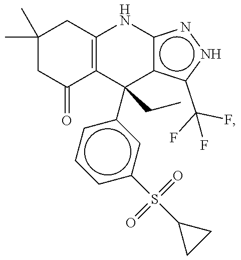 | 945713 |
| 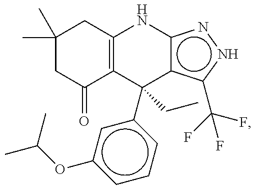 | 947867 |
| 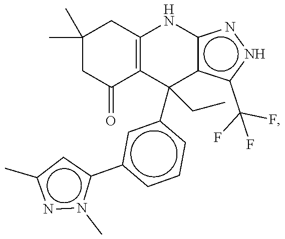 | 945532 |
| 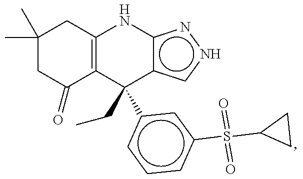 | 912171 |
| 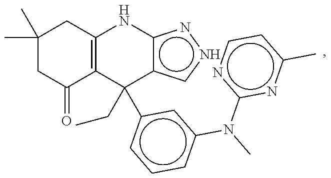 | 942642 |
| 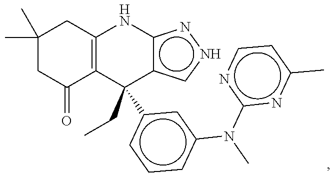 | 945328 |
| 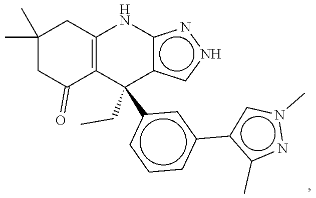 | 945546 |
| 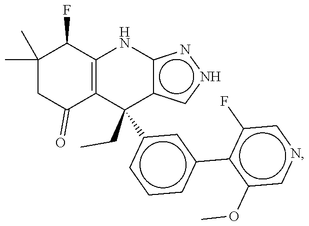 | 948545 |
| 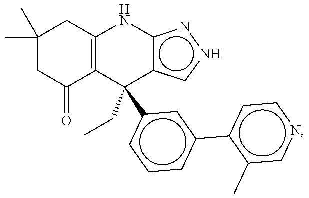 | 913047 |
| 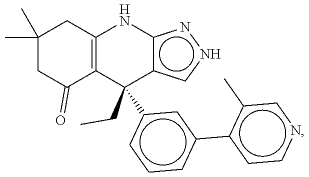 | 943990 |
| 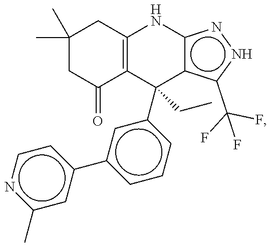 | 917291 |
| 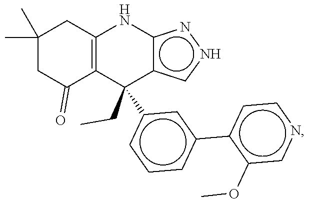 | 945481 |
| 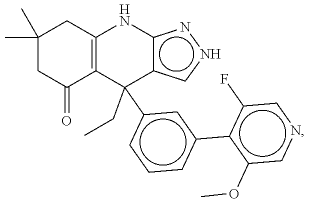 | 945704 |
| 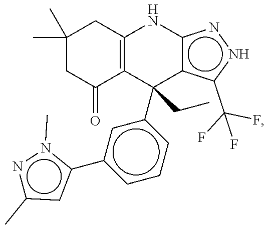 | 947384 |
| 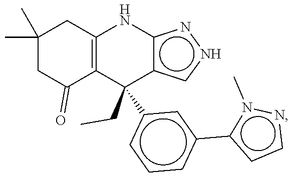 | 911992 |
| 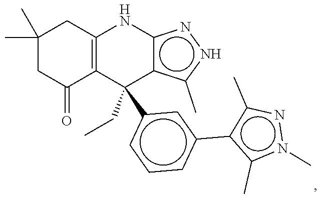 | 913043 |
| 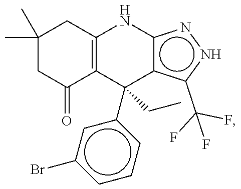 | 810366 |
| 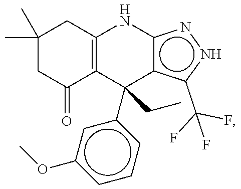 | 884117 |
| 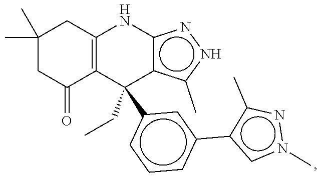 | 913035 |
| 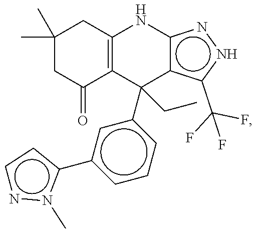 | 945531 |
| 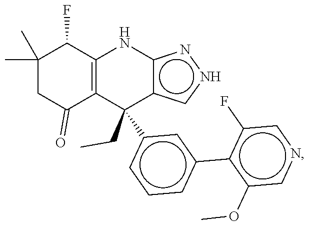 | 948546 |
| 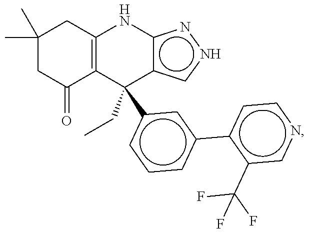 | 943913 |
| 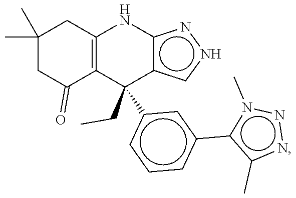 | 946099 |
| 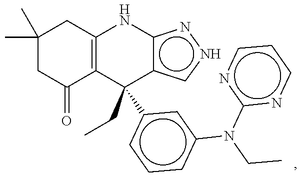 | 947333 |
| 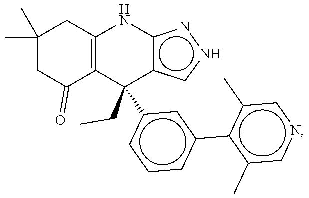 | 947658 |
| 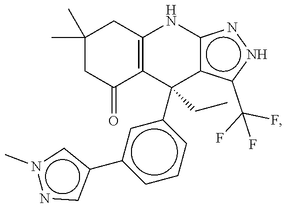 | 811565 |
| 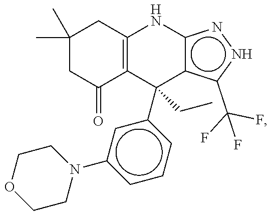 | 816885 |
| 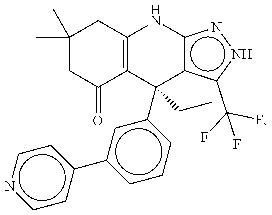 | 917293 |
| 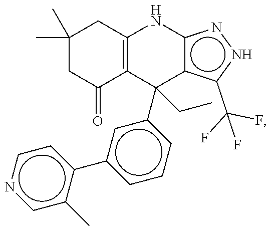 | 945527 |
| 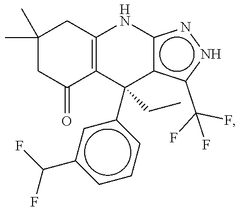 | 945710 |
| 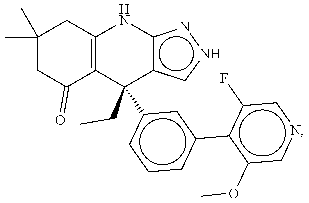 | 947511 |
| 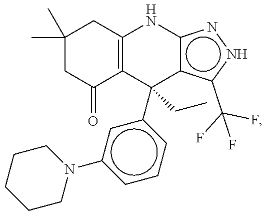 | 816800 |
| 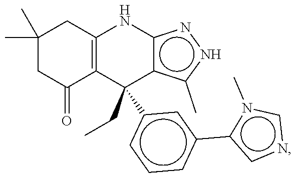 | 913032 |
| 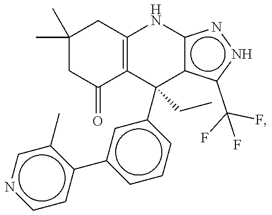 | 947324 |
| 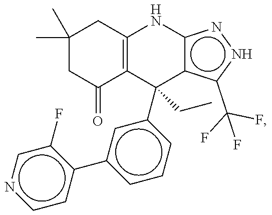 | 947326 |
| 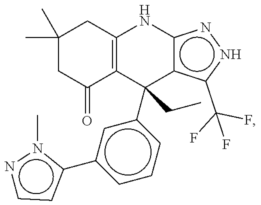 | 947372 |
| 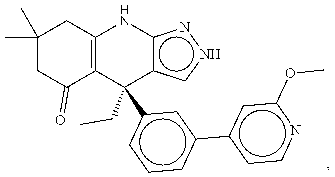 | 911989 |
| 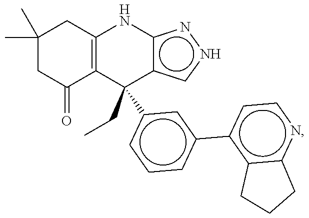 | 945619 |
| 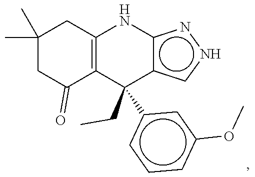 | 811561 |
| 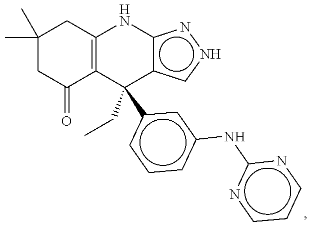 | 945958 |
| 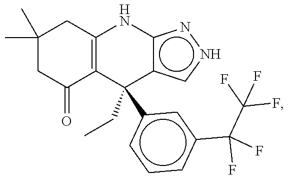 | 949268 |
| 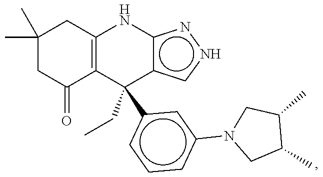 | 918327 |
| 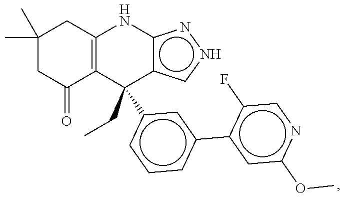 | 944035 |
| 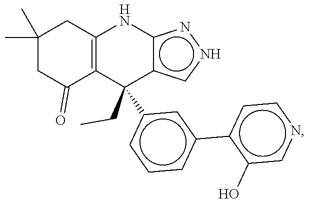 | 945728 |
| 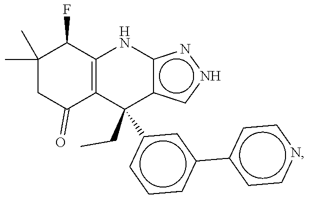 | 959342 |
| 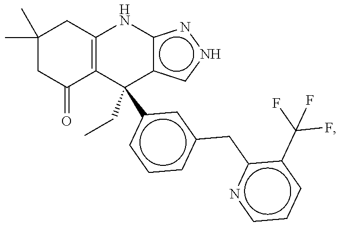 | 947665 |
| 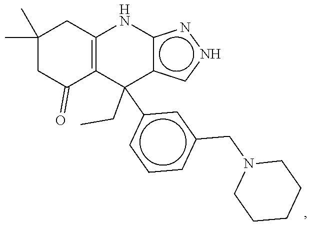 | 819560 |
| 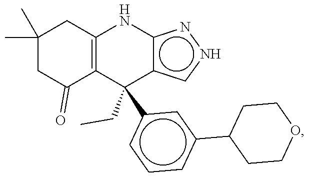 | 819639 |
| 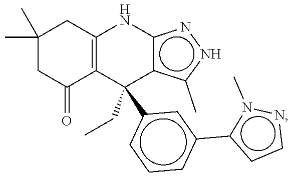 | 913046 |
| 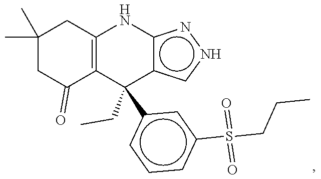 | 929452 |
| 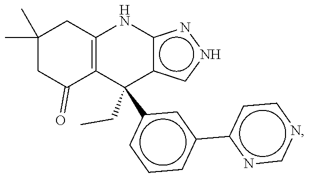 | 810489 |
| 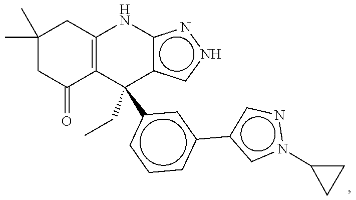 | 913041 |
| 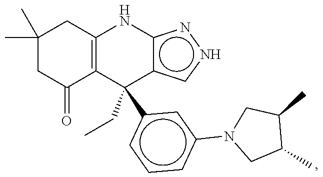 | 924543 |
| 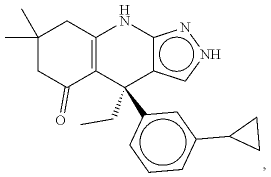 | 811558 |
| 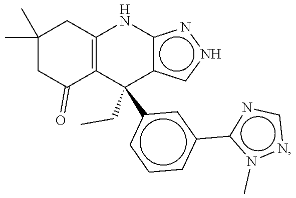 | 945482 |
| 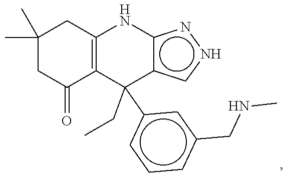 | 826651 |
| 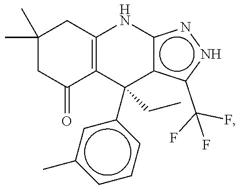 | 883872 |
| 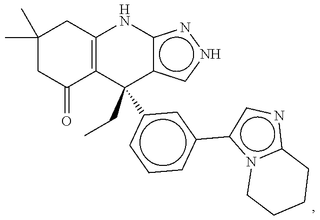 | 945618 |
| 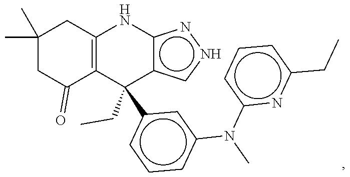 | 945726 |
| 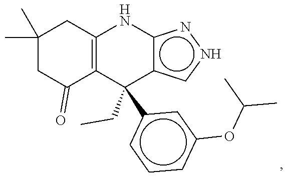 | 811563 |
| 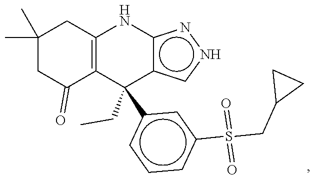 | 917151 |
| 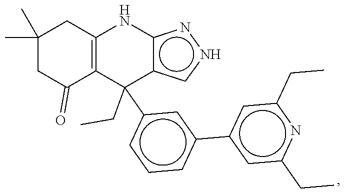 | 929445 |
| 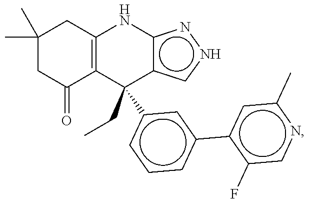 | 927339 |
| 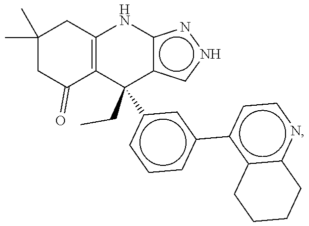 | 944036 |
| 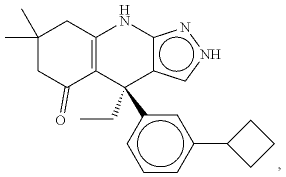 | 949514 |
| 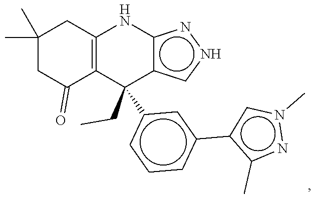 | 945545 |
| 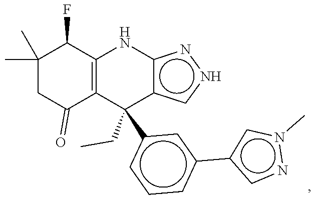 | 957510 |
| 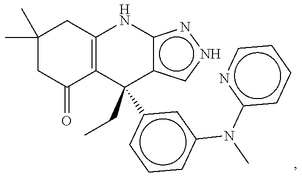 | 875608 |
| 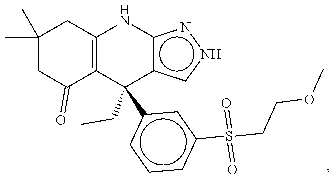 | 943546 |
| 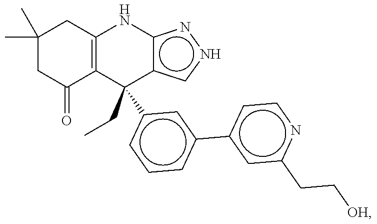 | 947816 |
| 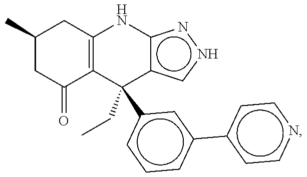 | 874315 |
| 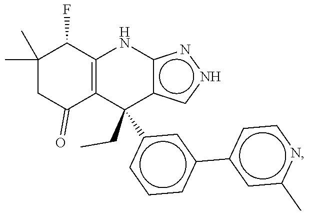 | 946101 |
| 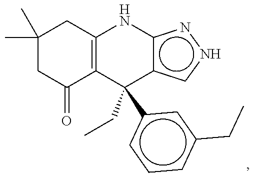 | 949391 |
| 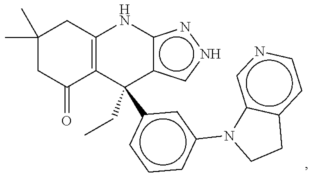 | 912004 |
| 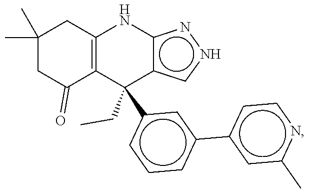 | 865170 |
| 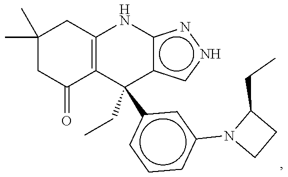 | 911972 |
| 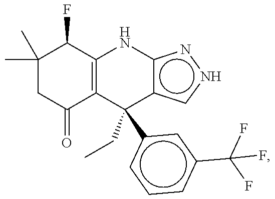 | 837803 |
| 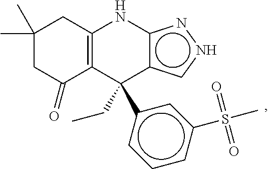 | 826719 |
| 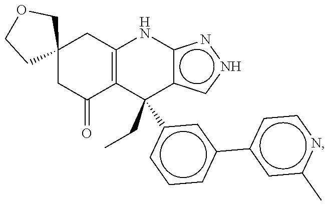 | 947412 |
| 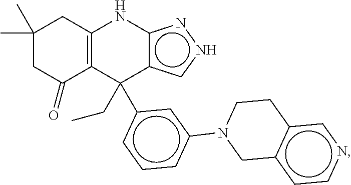 | 929449 |
| 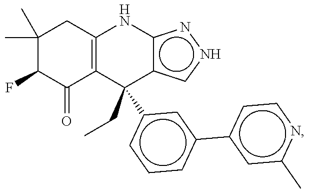 | 945486 |
| 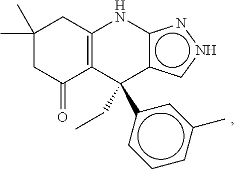 | 789561 |
| 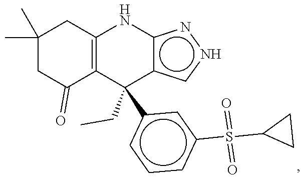 | 9121272 |
| 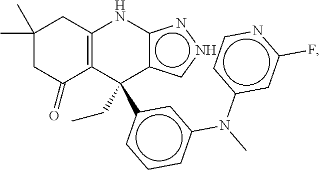 | 929453 |
| 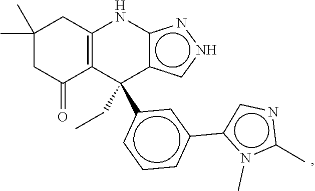 | 945612 |
| 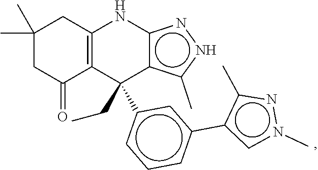 | 913034 |
| 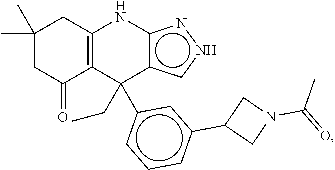 | 948779 |
| 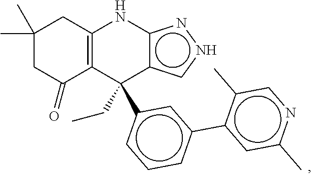 | 943515 |
| 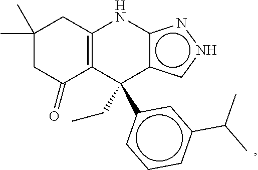 | 810375 |
| 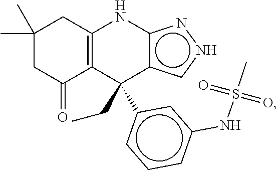 | 927341 |
| 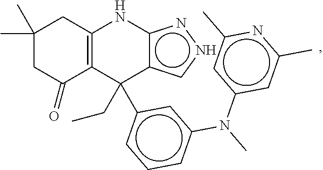 | 913256 |
| 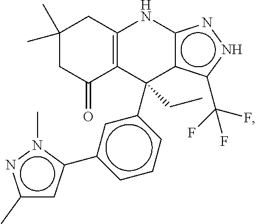 | 947383 |
| 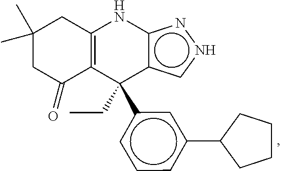 | 949399 |
| 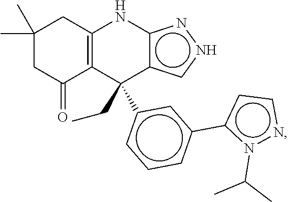 | 946092 |
| 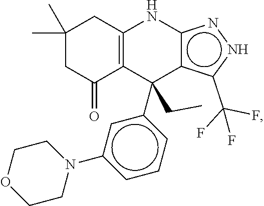 | 816886 |
| 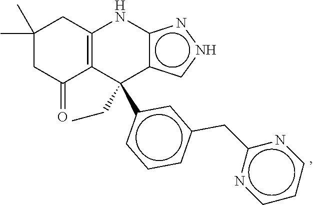 | 947499 |
| 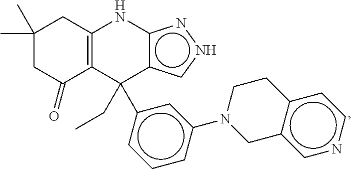 | 917148 |
| 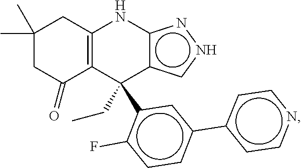 | 893324 |
| 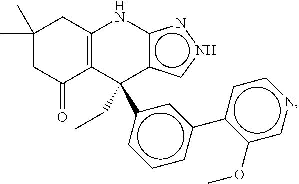 | 945471 |
| 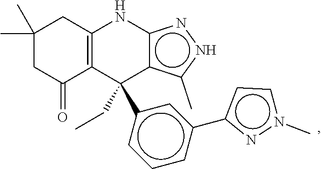 | 913039 |
| 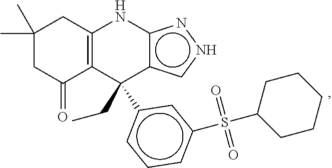 | 943547 |
| 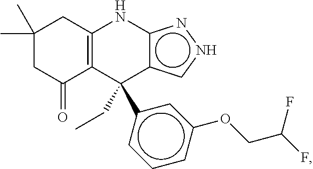 | 949113 |
| 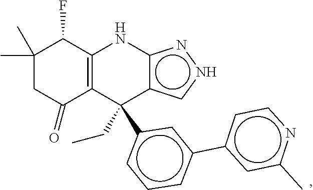 | 946102 |
| 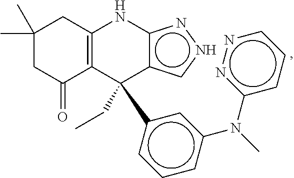 | 911976 |
| 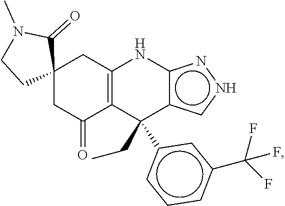 | 950330 |
| 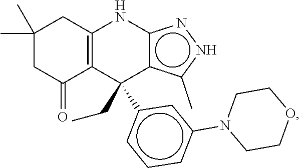 | 889582 |
| 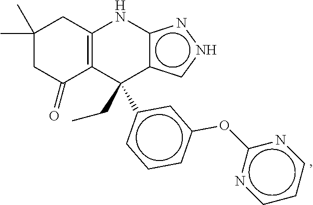 | 947818 |
| 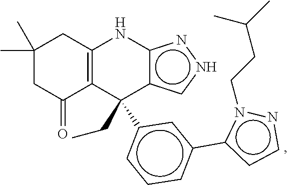 | 946096 |
| 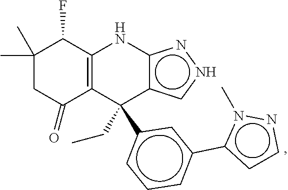 | 957509 |
| 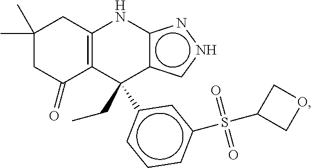 | 946003 |
| 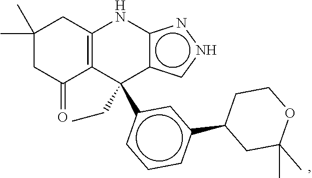 | 912090 |
| 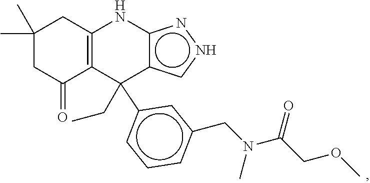 | 865196 |
| 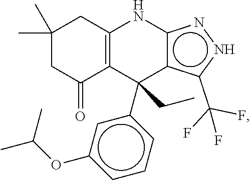 | 947872 |
| 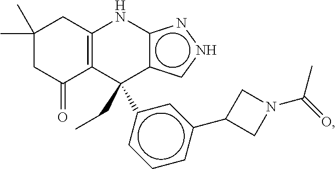 | 949131 |
| 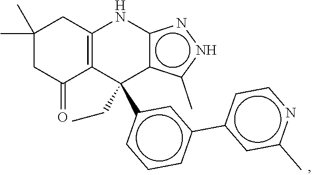 | 913051 |
| 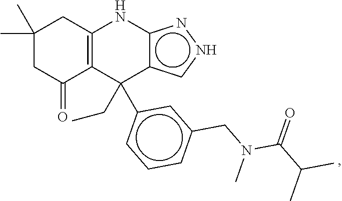 | 865195 |
| 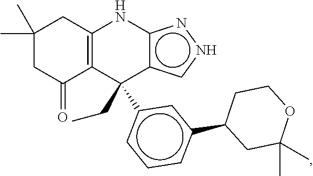 | 912088 |
| 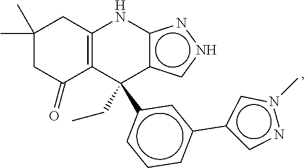 | 819576 |
| 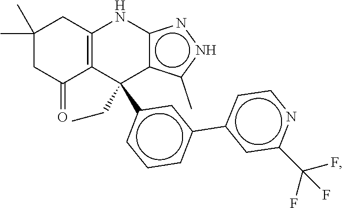 | 913049 |
| 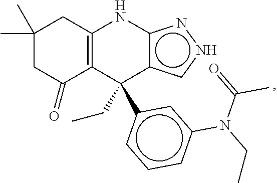 | 909215 |
| 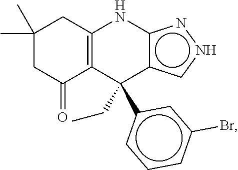 | 790734 |
| 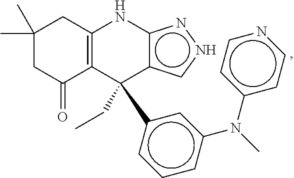 | 874261 |
| 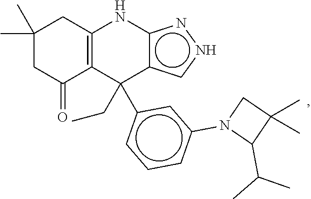 | 902969 |
| 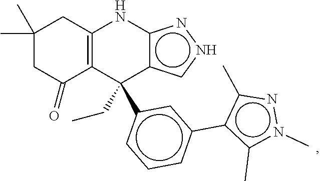 | 911991 |
| 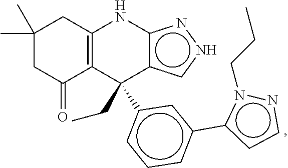 | 946094 |
| 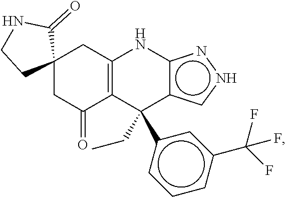 | 950331 |
| 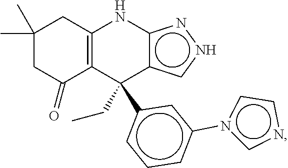 | 874533 |
| 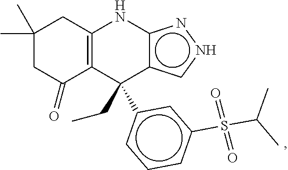 | 913134 |
| 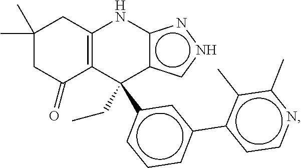 | 943517 |
| 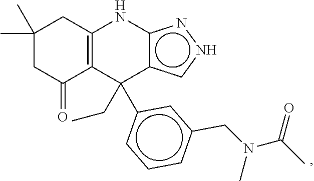 | 865194 |
| 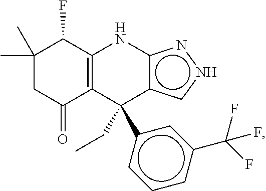 | 837753 |
| 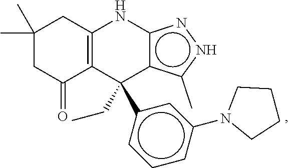 | 883875 |
| 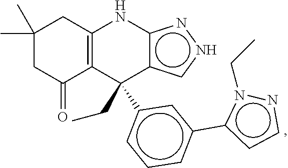 | 946090 |
| 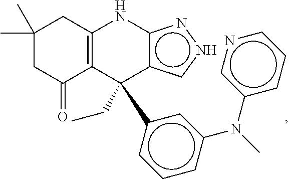 | 874299 |
| 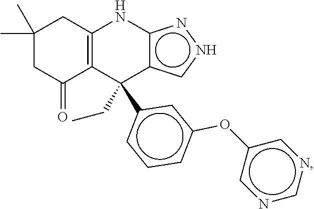 | 945939 |
| 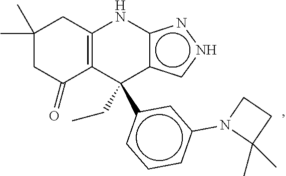 | 913141 |
| 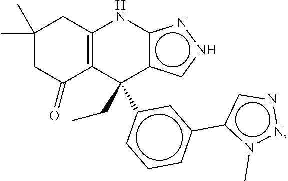 | 947824 |
| 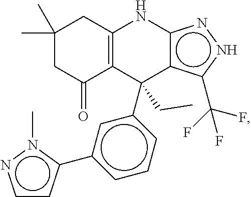 | 947365 |
| 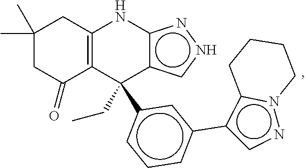 | 945544 |
| 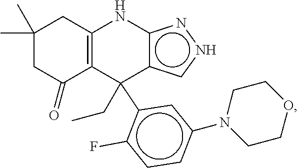 | 909203 |
| 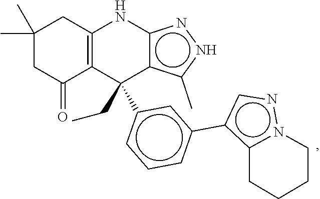 | 913033 |
| 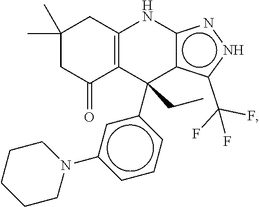 | 816799 |
| 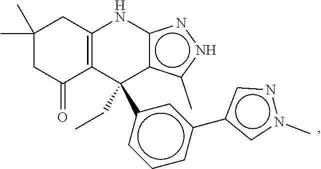 | 913038 |
| 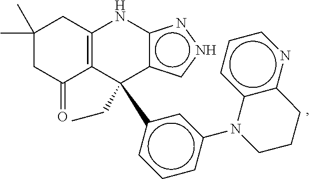 | 917079 |
| 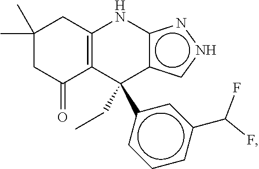 | 826716 |
| 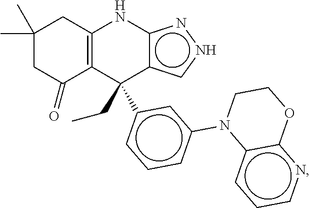 | 932643 |
| 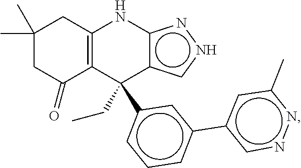 | 943995 |
| 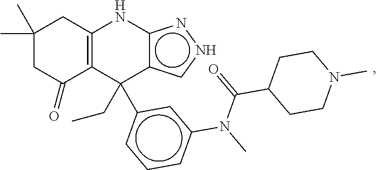 | 912167 |
| 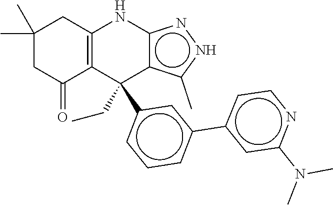 | 913048 |
| 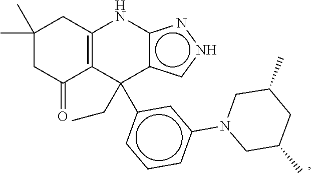 | 885237 |
| 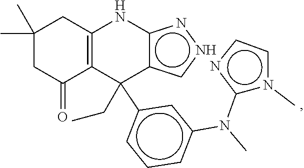 | 885402 |
| 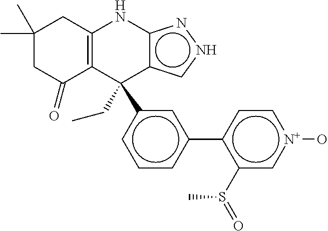 | 947571 |
| 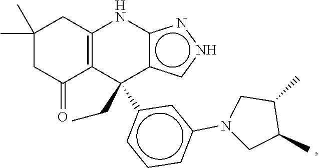 | 924312 |
| 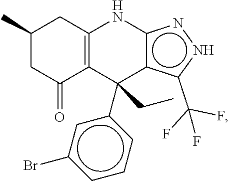 | 828850 |
| 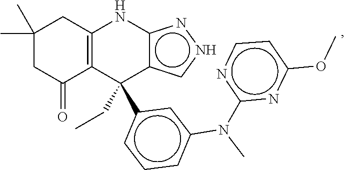 | 947337 |
| 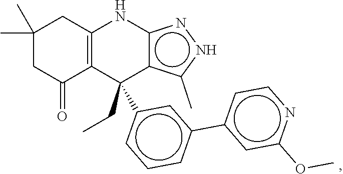 | 913016 |
| 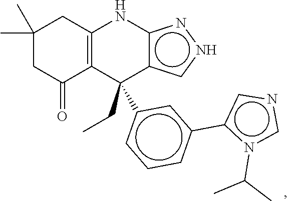 | 945729 |
| 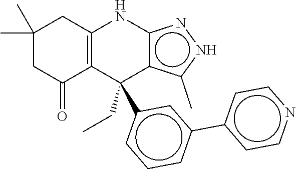 | 913054 |
| 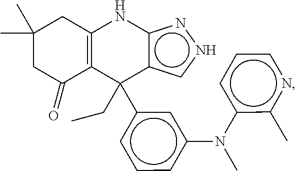 | 885355 |
| 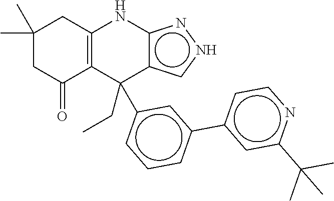 | 947490 |
| 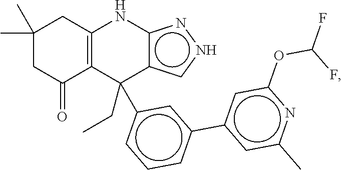 | 945463 |
| 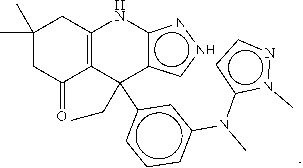 | 889604 |
| 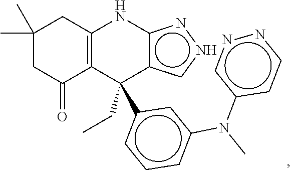 | 915630 |
| 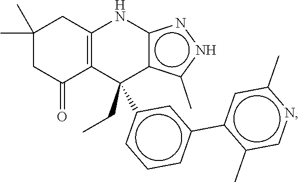 | 913019 |
| 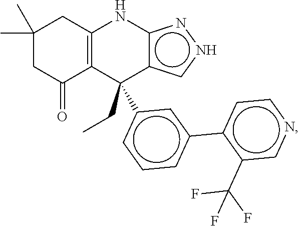 | 943918 |
| 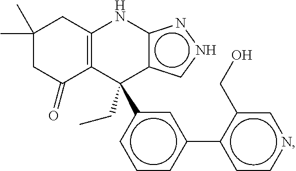 | 945479 |
| 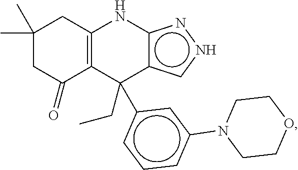 | 801318 |
| 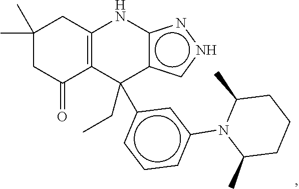 | 865172 |
| 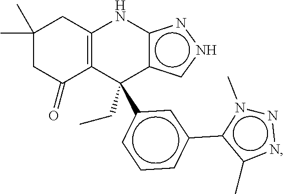 | 946100 |
| 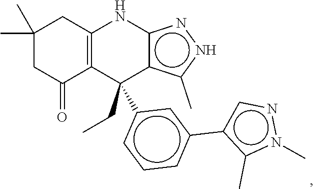 | 913024 |
| 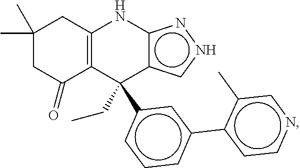 | 943991 |
| 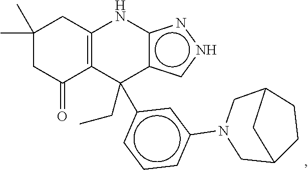 | 885099 |
| 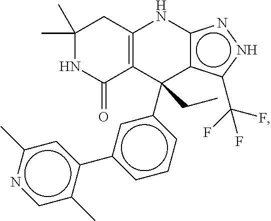 | 947364 |
| 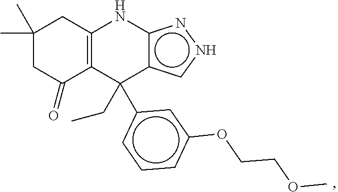 | 801446 |
| 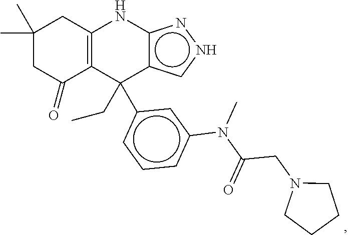 | 909202 |
| 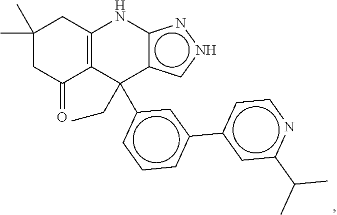 | 930569 |
| 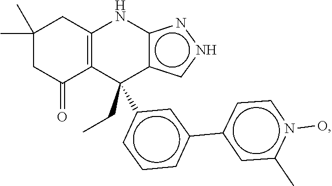 | 946055 |
| 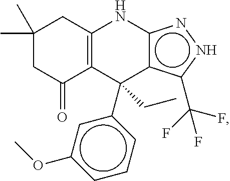 | 884118 |
| 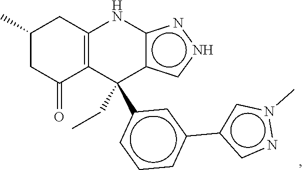 | 823724 |
| 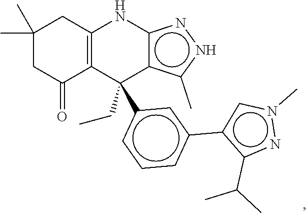 | 913040 |
| 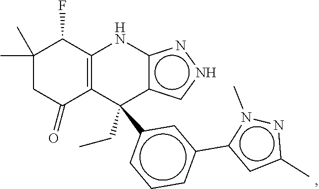 | 947657 |
| 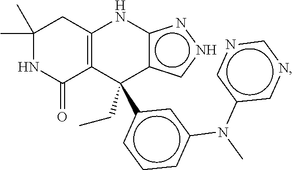 | 915401 |
| 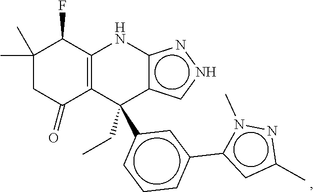 | 947656 |
| 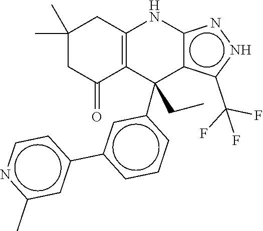 | 917289 |
| 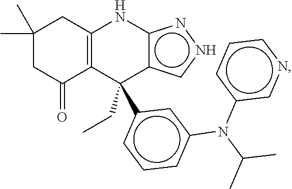 | 917087 |
| 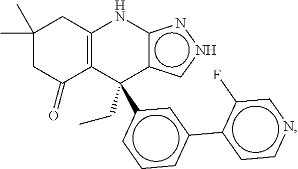 | 943519 |
| 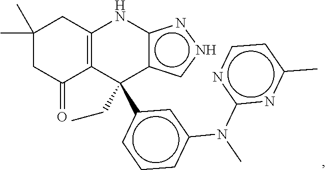 | 945329 |
| 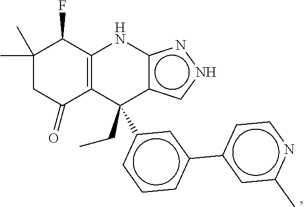 | 946103 |
| 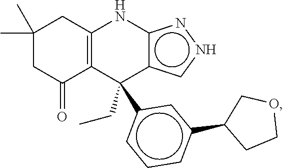 | 889565 |
| 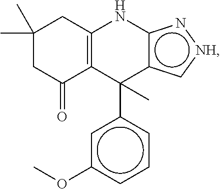 | 792342 |
| 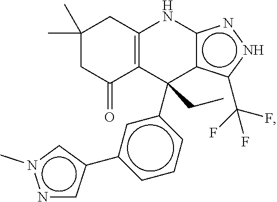 | 811567 |
| 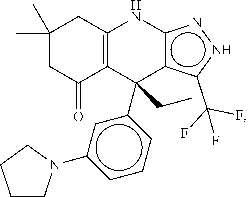 | 819575 |
| 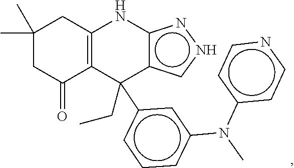 | 853910 |
| 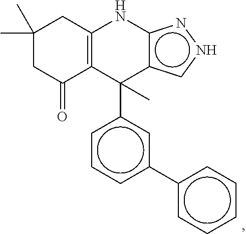 | 789513 |
| 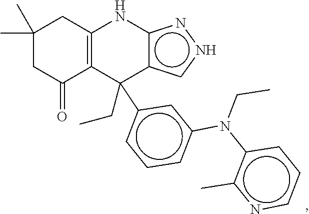 | 885367 |
| 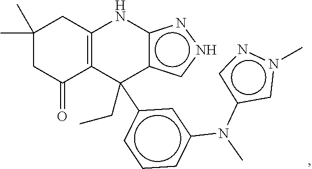 | 885382 |
| 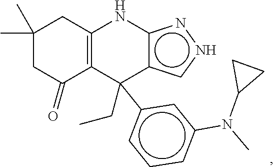 | 945466 |
| 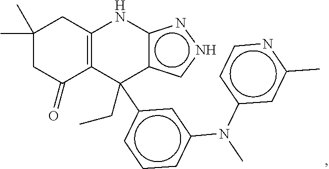 | 917149 |
| 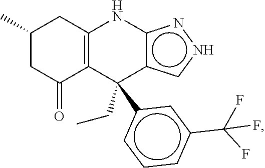 | 810452 |
| 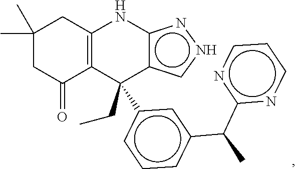 | 948057 |
| 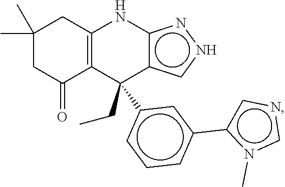 | 945620 |
| 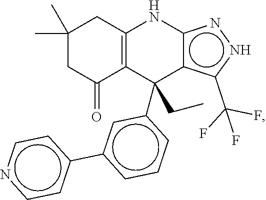 | 917292 |
| 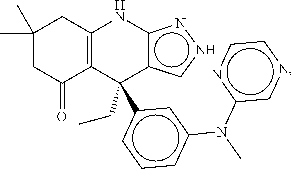 | 915340 |
| 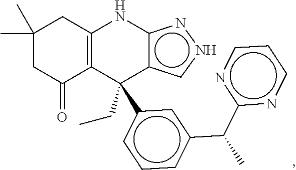 | 948061 |
| 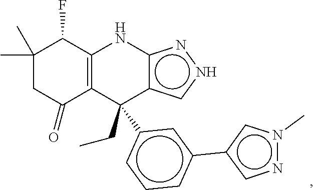 | 957504 |
| 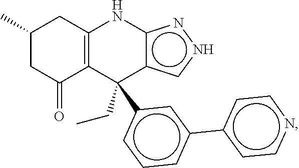 | 874316 |
| 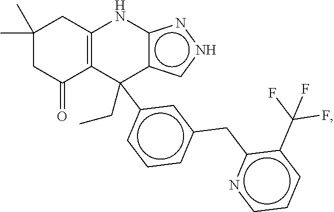 | 947548 |
| 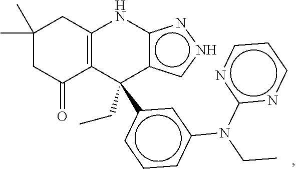 | 947334 |
| 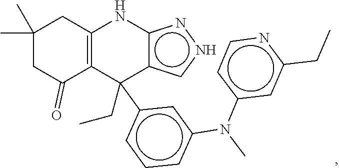 | 945600 |
| 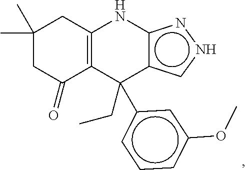 | 801447 |
| 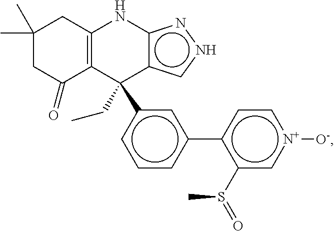 | 947570 |
| 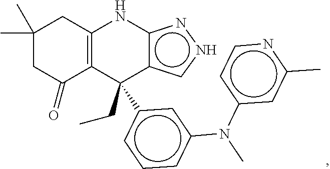 | 932645 |
| 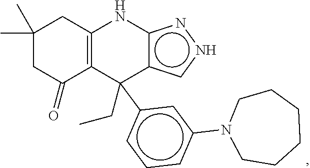 | 885088 |
| 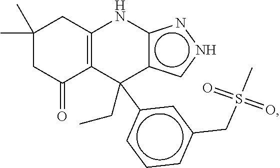 | 945601 |
| 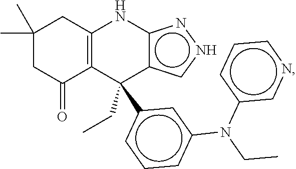 | 917089 |
| 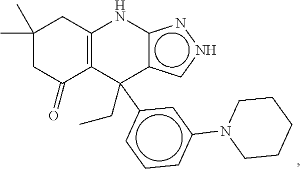 | 801320 |
| 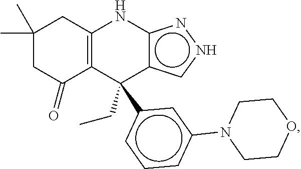 | 810363 |
| 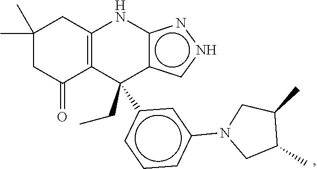 | 924504 |
| 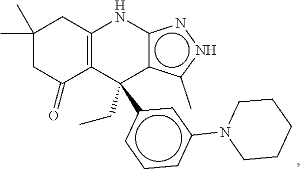 | 884115 |
| 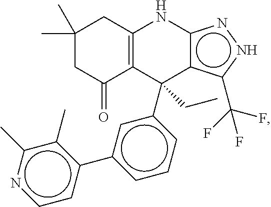 | 947328 |
| 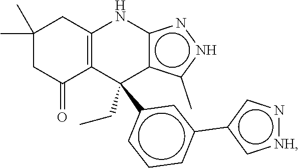 | 913044 |
| 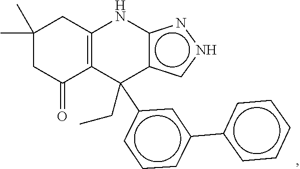 | 789514 |
| 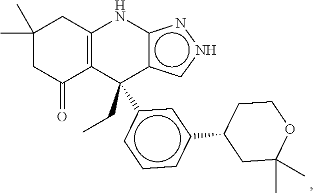 | 912089 |
| 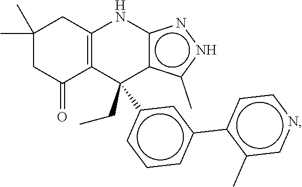 | 913022 |
| 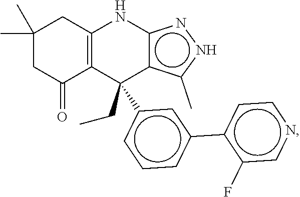 | 913017 |
| 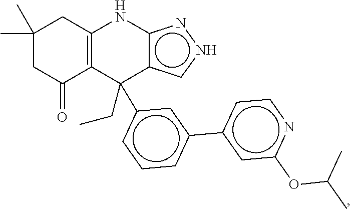 | 932626 |
| 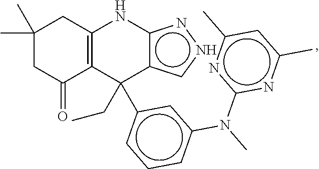 | 926304 |
| 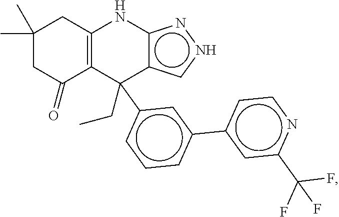 | 913350 |
| 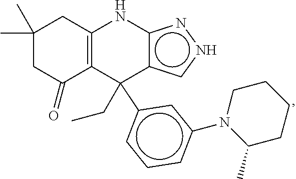 | 885217 |
| 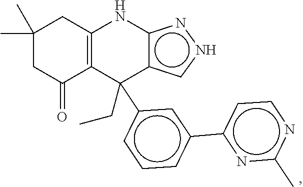 | 948088 |
| 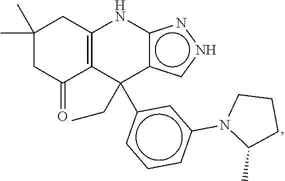 | 885188 |
| 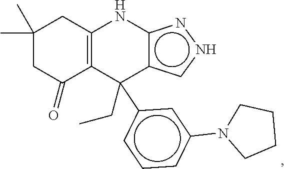 | 801317 |
| 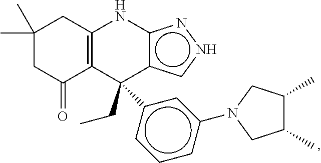 | 918326 |
| 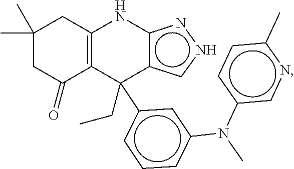 | 918272 |
| 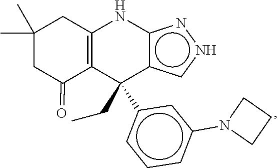 | 889631 |
| 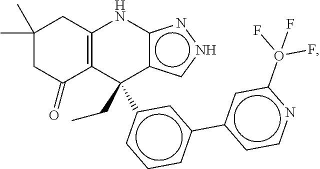 | 943988 |
| 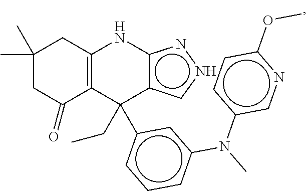 | 913252 |
| 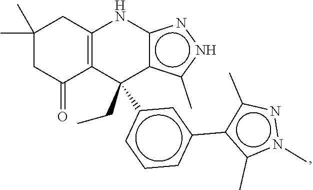 | 913026 |
| 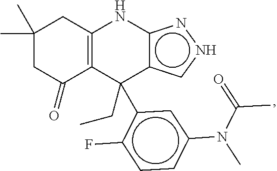 | 913124 |
| 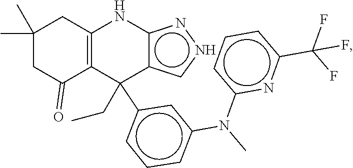 | 913122 |
| 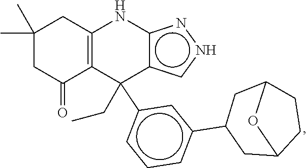 | 889622 |
| 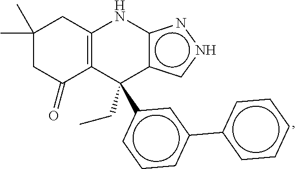 | 801368 |
| 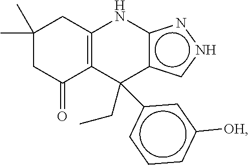 | 930334 |
| 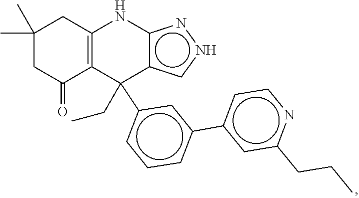 | 945467 |
| 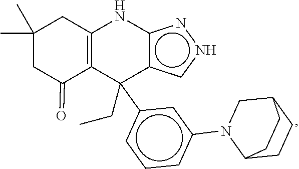 | 885120 |
| 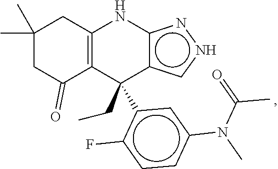 | 929450 |
| 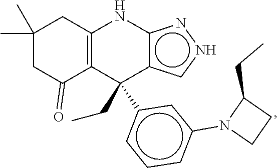 | 911969 |
| 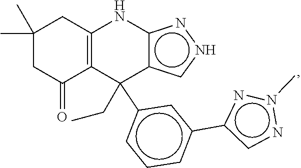 | 947492 |
| 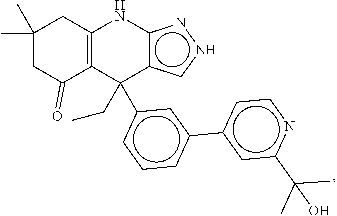 | 943536 |
| 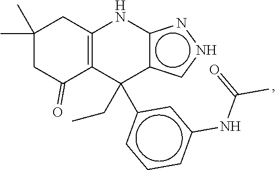 | 877310 |
| 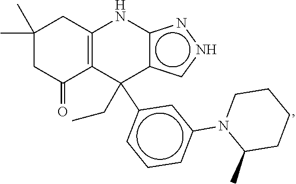 | 885228 |
| 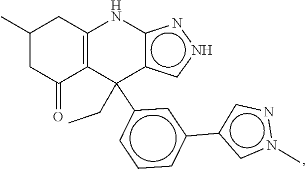 | 816752 |
| 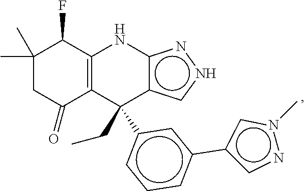 | 957505 |
| 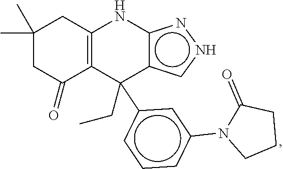 | 865171 |
| 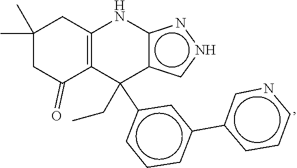 | 791272 |
| 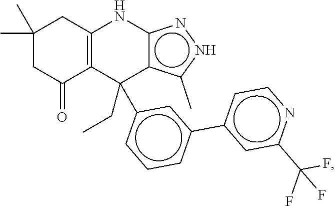 | 883989 |
| 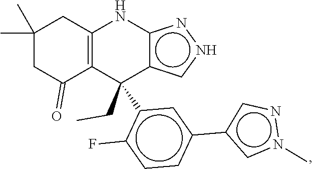 | 909220 |
| 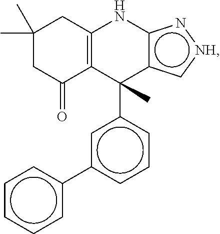 | 801367 |
| 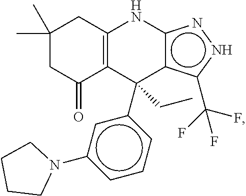 | 819574 |
| 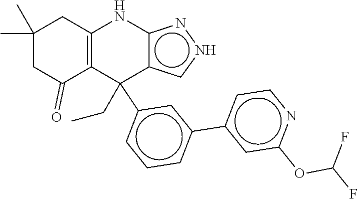 | 932629 |
| 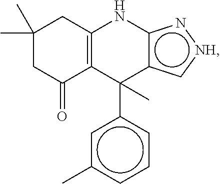 | 785736 |
| 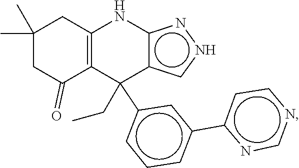 | 806637 |
| 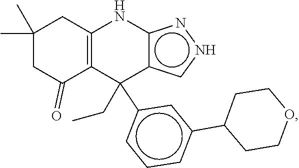 | 791273 |
| 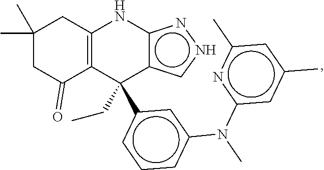 | 945610 |
| 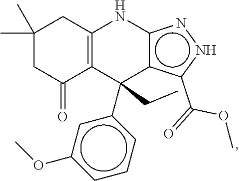 | 901480 |
| 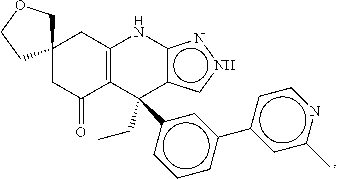 | 947407 |
| 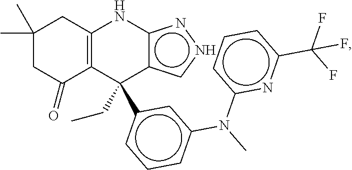 | 927338 |
| 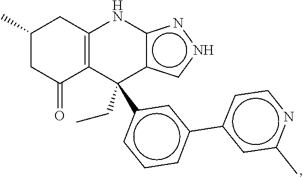 | 932657 |
| 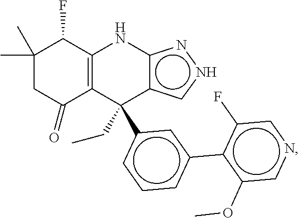 | 948548 |
| 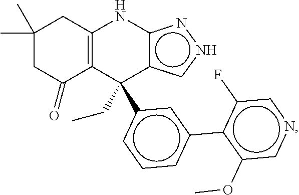 | 947512 |
| 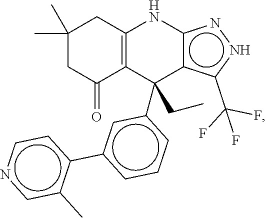 | 947325 |
| 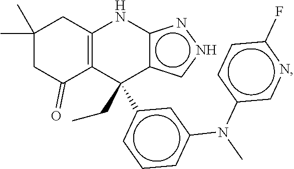 | 932647 |
| 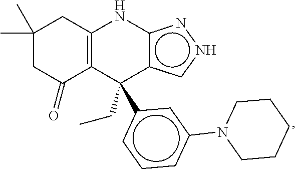 | 810369 |
| 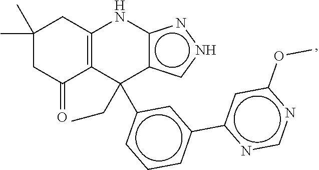 | 948093 |
| 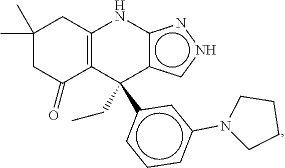 | 810510 |
| 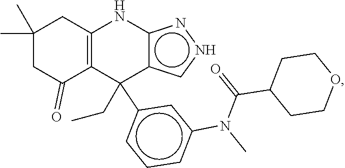 | 912070 |
| 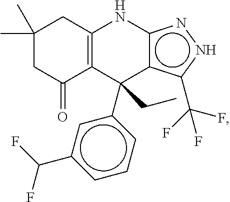 | 945708 |
| 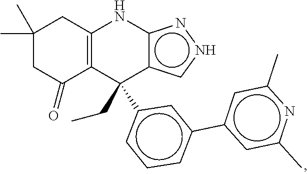 | 913222 |
| 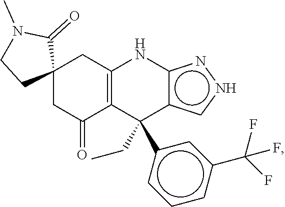 | 950326 |
| 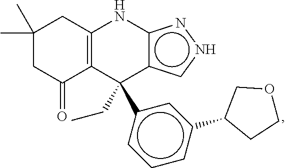 | 889558 |
| 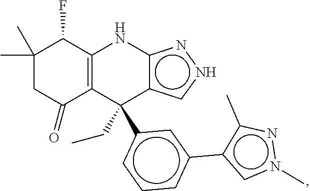 | 947653 |
| 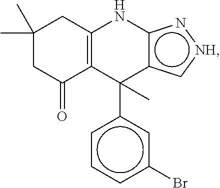 | 789277 |
| 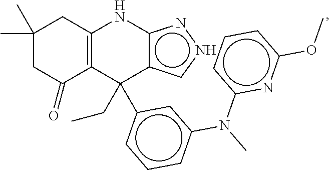 | 913121 |
| 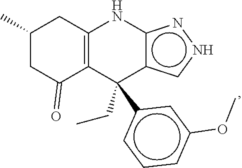 | 811559 |
| 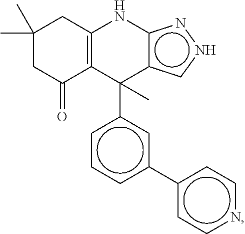 | 790730 |
| 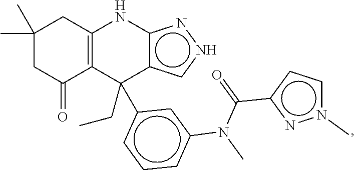 | 917130 |
| 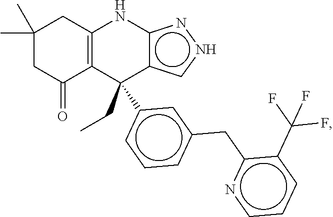 | 947664 |
| 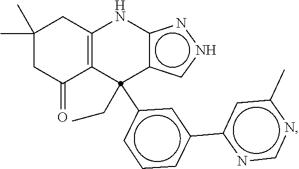 | 948089 |
| 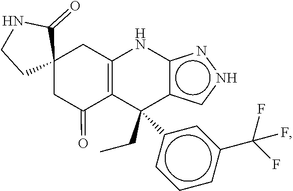 | 950333 |
| 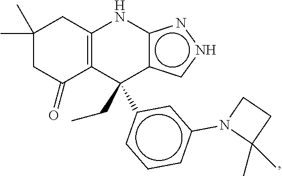 | 913140 |
| 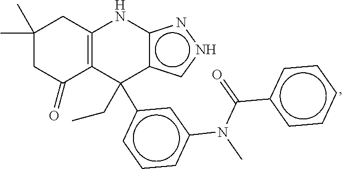 | 909201 |
| 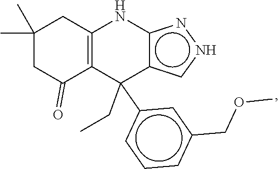 | 801323 |
| 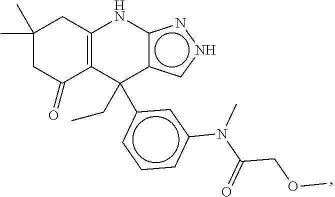 | 889580 |
| 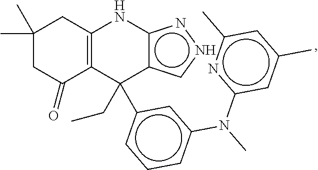 | 943538 |
| 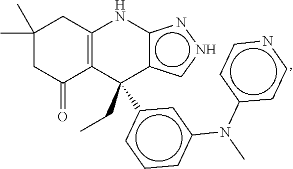 | 874260 |
| 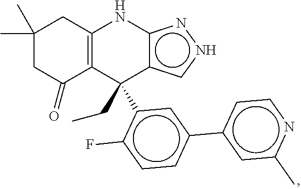 | 909212 |
| 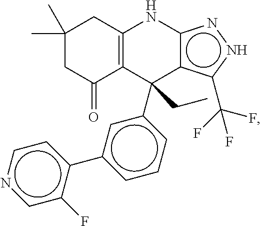 | 947327 |
| 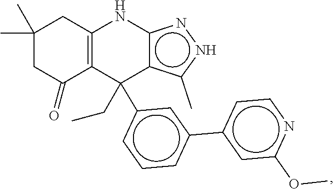 | 883985 |
| 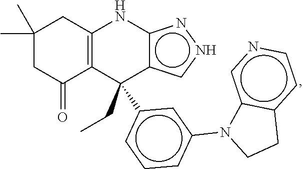 | 912003 |
| 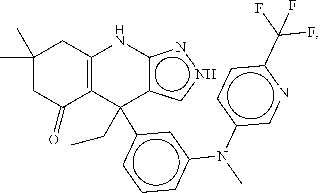 | 932634 |
| 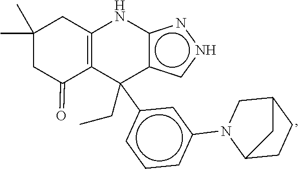 | 885110 |
| 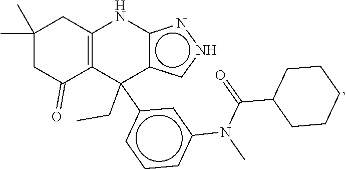 | 912065 |
| 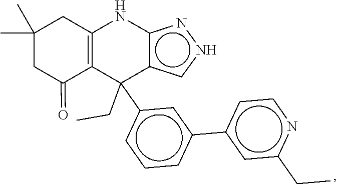 | 947493 |
| 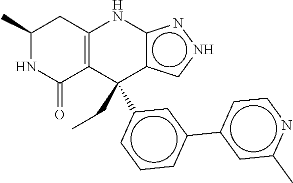 | 932658 |
| 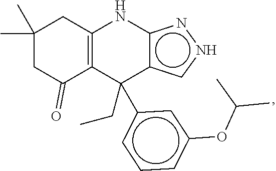 | 801448 |
| 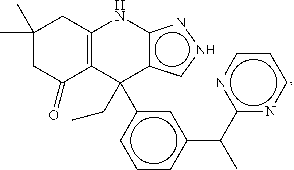 | 947434 |
| 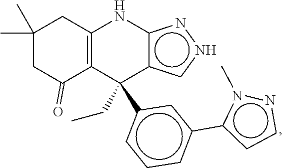 | 911993 |
| 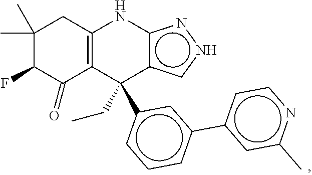 | 945475 |
| 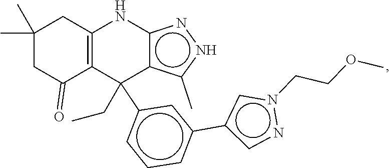 | 883996 |
| 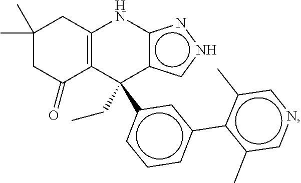 | 947659 |
| 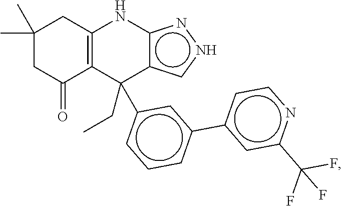 | 913348 |
| 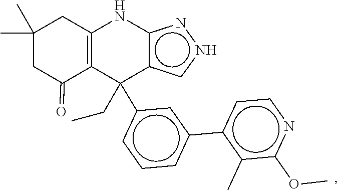 | 932628 |
| 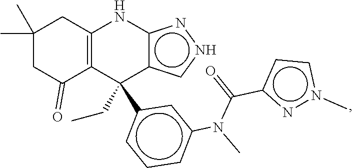 | 929442 |
| 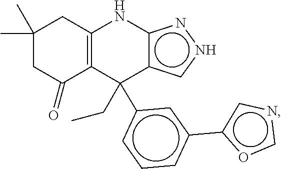 | 944025 |
| 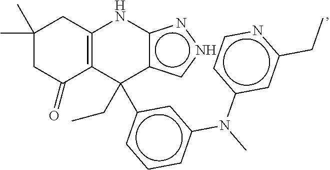 | 917147 |
| 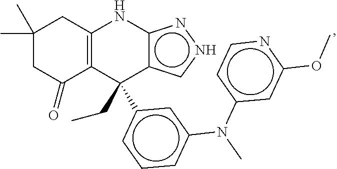 | 932642 |
| 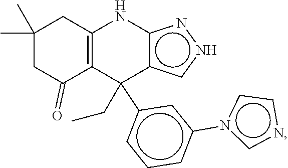 | 854926 |
| 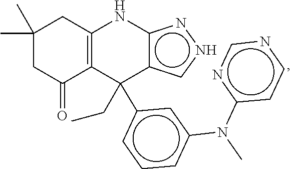 | 902970 |
| 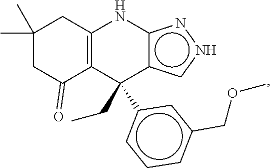 | 810360 |
| 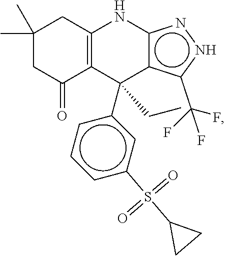 | 945715 |
| 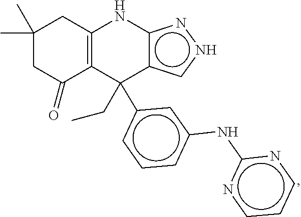 | 913334 |
| 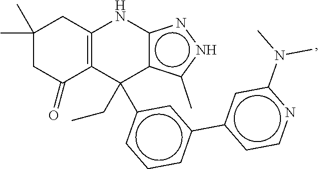 | 883990 |
| 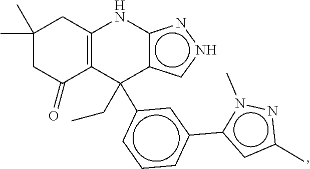 | 929402 |
| 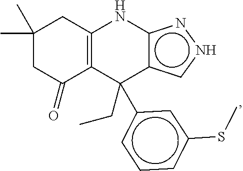 | 810508 |
| 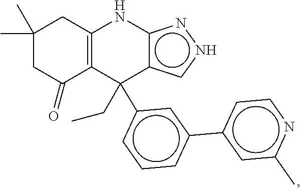 | 840857 |
| 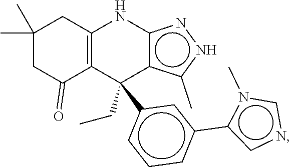 | 913037 |
| 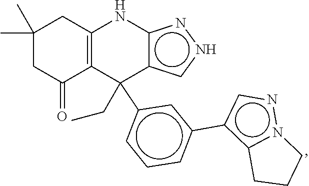 | 947494 |
| 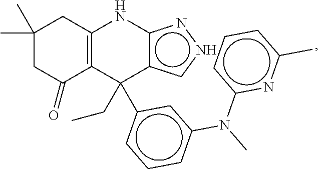 | 913255 |
| 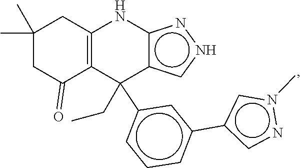 | 816871 |
| 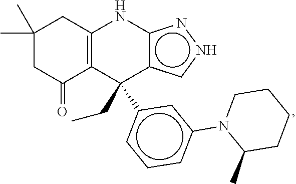 | 917209 |
| 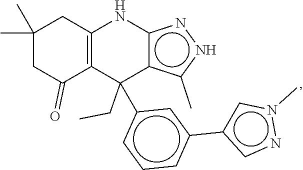 | 884000 |
| 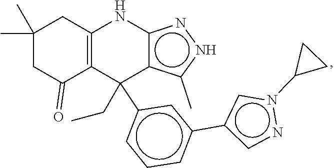 | 883997 |
| 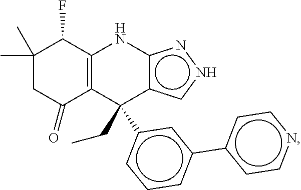 | 959341 |
| 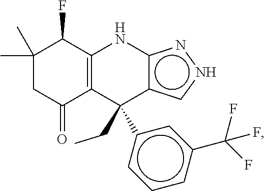 | 837683 |
| 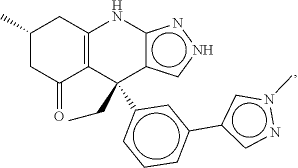 | 823720 |
| 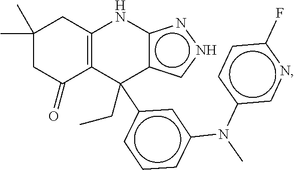 | 917152 |
| 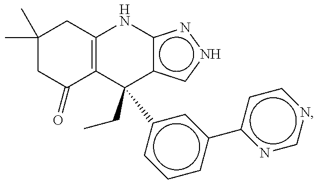 | 810488 |
| 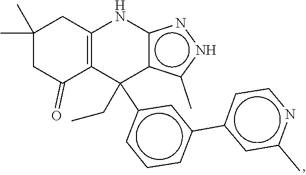 | 883987 |
| 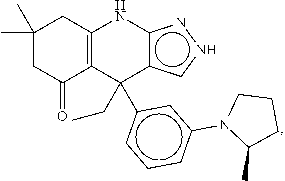 | 885204 |
| 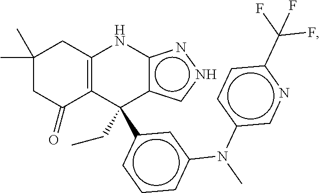 | 945474 |
| 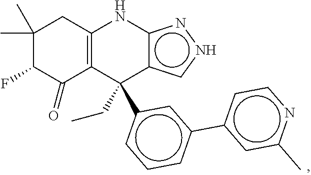 | 945476 |
| 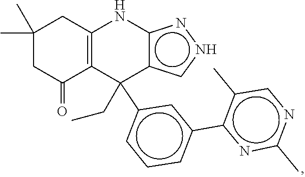 | 948091 |
| 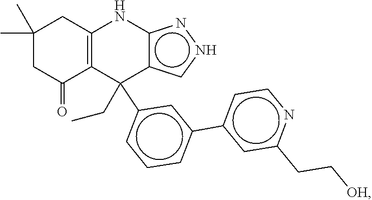 | 945992 |
| 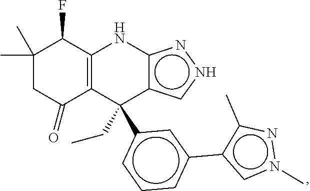 | 947652 |
| 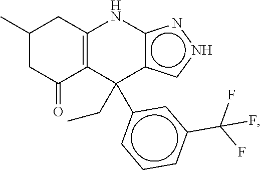 | 801397 |
| 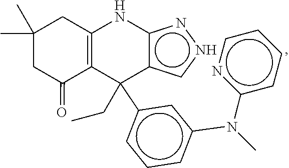 | 874294 |
| 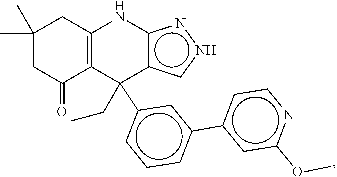 | 883850 |
| 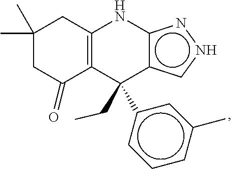 | 789560 |
| 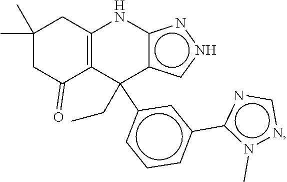 | 943532 |
| 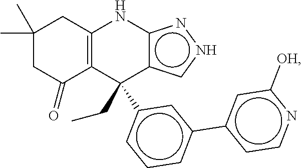 | 943992 |
| 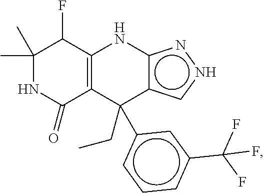 | 819622 |
| 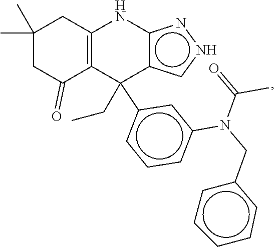 | 884110 |
| 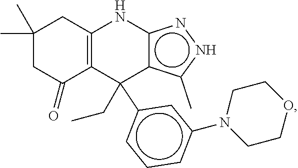 | 874312 |
| 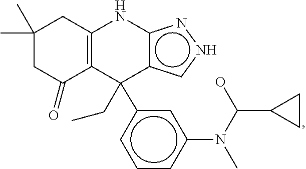 | 912067 |
| 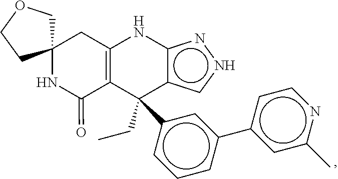 | 947406 |
| 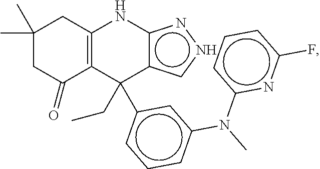 | 918274 |
| 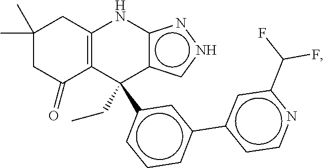 | 943919 |
| 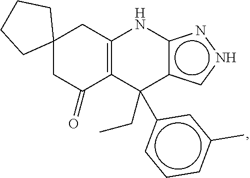 | 789565 |
| 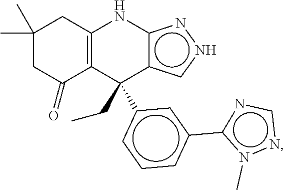 | 945472 |
| 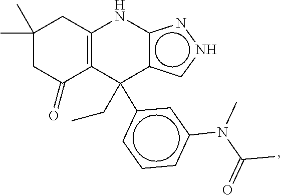 | 801319 |
| 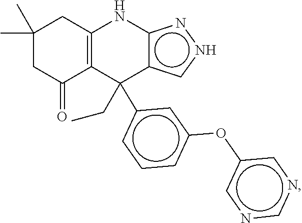 | 917212 |
| 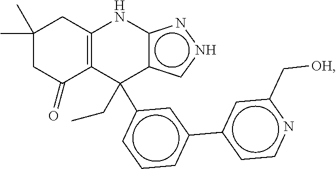 | 943540 |
| 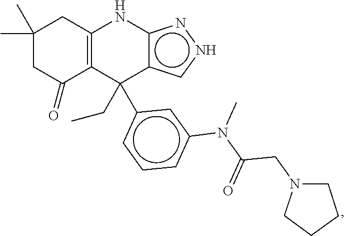 | 913125 |
| 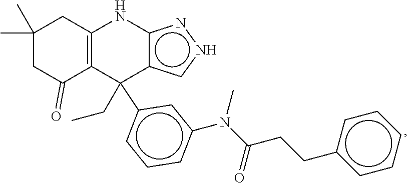 | 912066 |
| 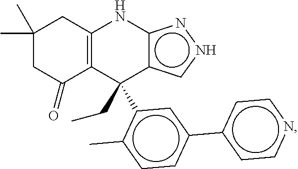 | 893323 |
| 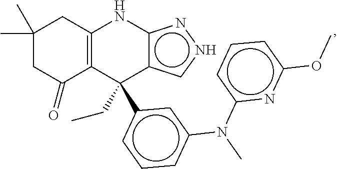 | 932666 |
| 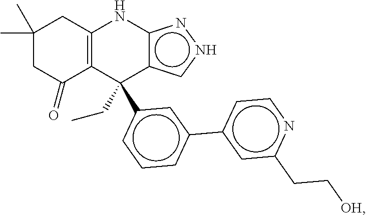 | 947810 |
| 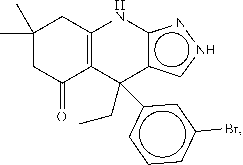 | 789312 |
| 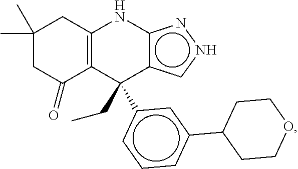 | 819638 |
| 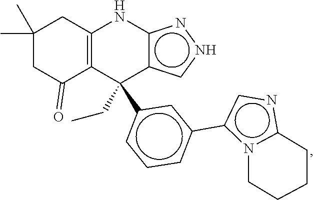 | 945607 |
| 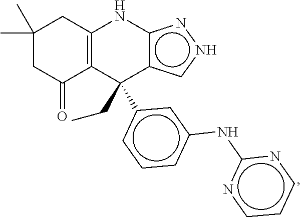 | 945943 |
| 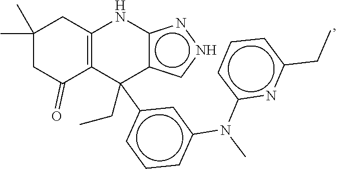 | 944023 |
| 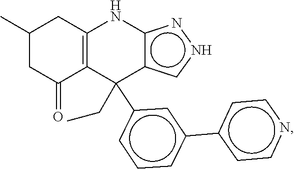 | 856947 |
| 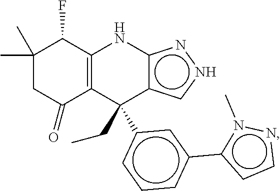 | 957506 |
| 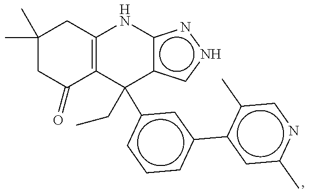 | 913346 |
| 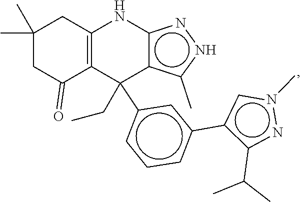 | 883998 |
| 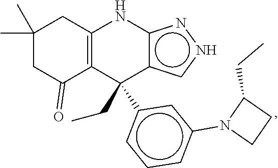 | 911970 |
| 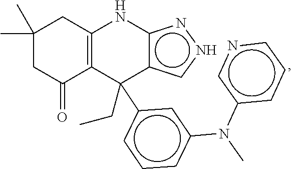 | 865168 |
| 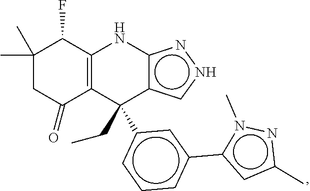 | 947655 |
| 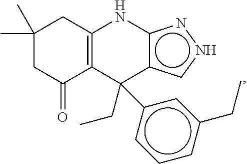 | 949083 |
| 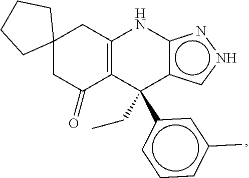 | 801312 |
| 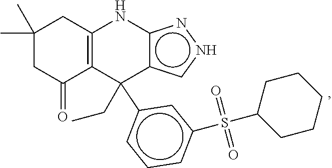 | 918273 |
| 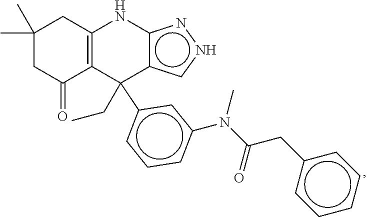 | 912071 |
| 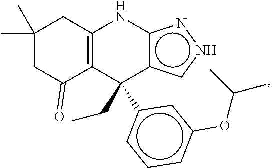 | 811564 |
| 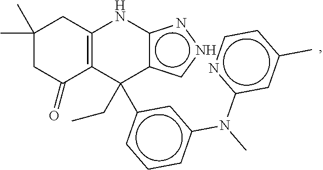 | 943537 |
| 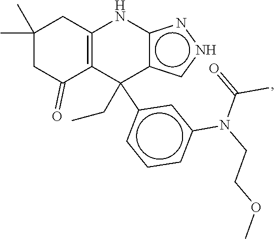 | 893306 |
| 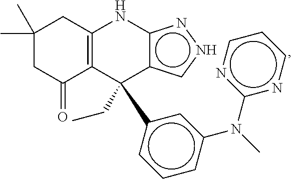 | 911981 |
| 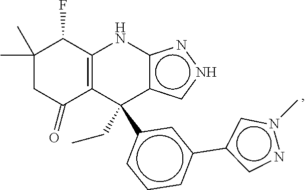 | 957511 |
| 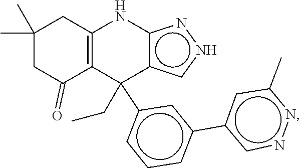 | 926844 |
| 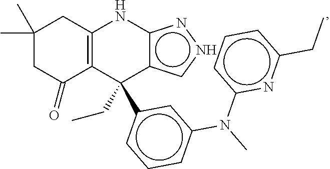 | 945730 |
| 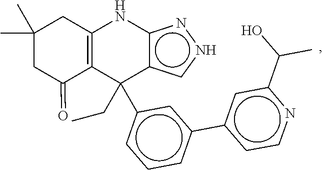 | 944029 |
| 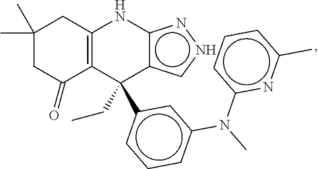 | 932664 |
| 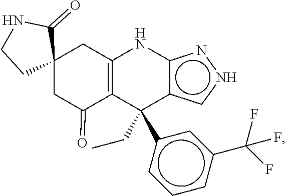 | 950324 |
| 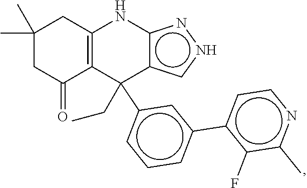 | 918281 |
| 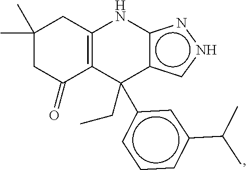 | 801322 |
| 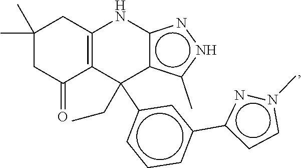 | 883999 |
| 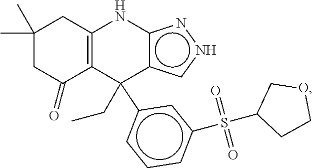 | 944018 |
| 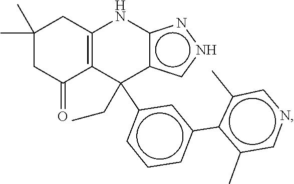 | 945703 |
| 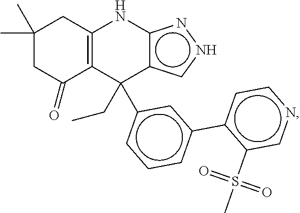 | 947569 |
| 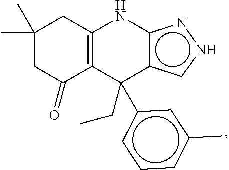 | 785738 |
| 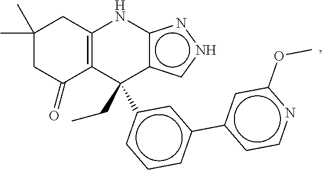 | 911988 |
| 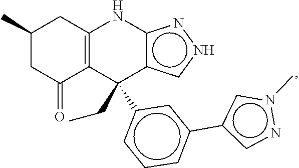 | 823721 |
| 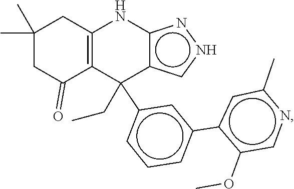 | 945468 |
| 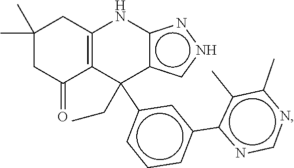 | 948090 |
| 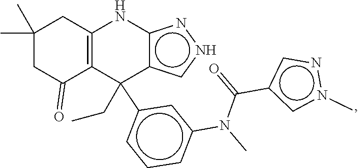 | 912068 |
| 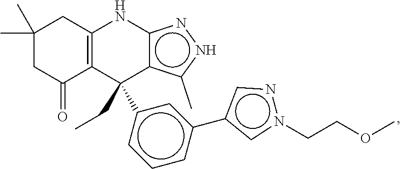 | 913027 |
| 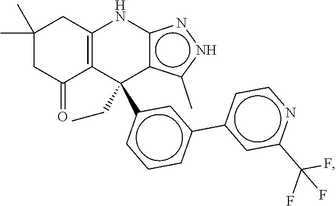 | 913020 |
| 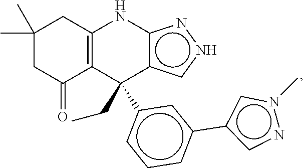 | 819577 |
| 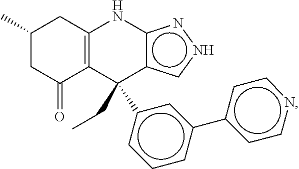 | 874318 |
| 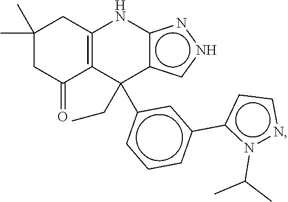 | 929399 |
| 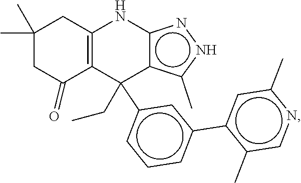 | 883988 |
| 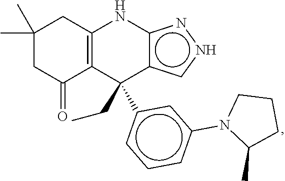 | 918321 |
| 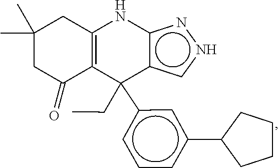 | 949082 |
| 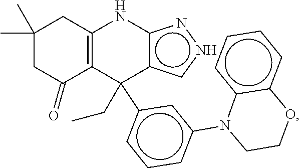 | 918280 |
| 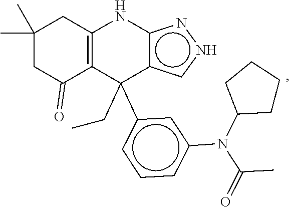 | 912069 |
| 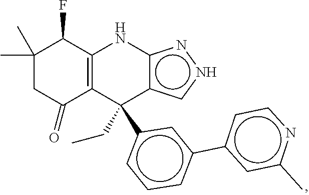 | 946104 |
| 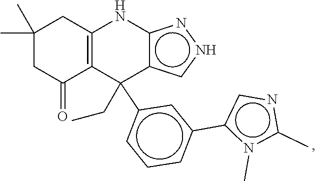 | 943531 |
| 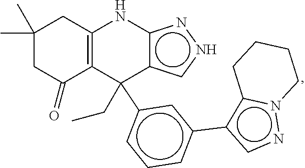 | 926836 |
| 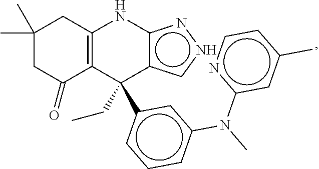 | 945609 |
| 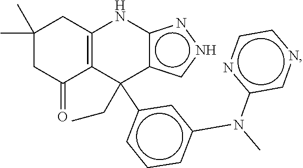 | 902971 |
| 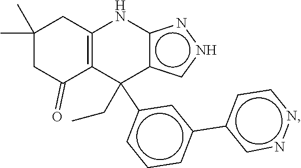 | 927342 |
| 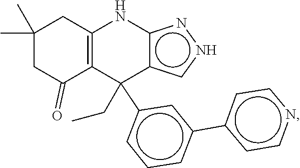 | 883984 |
| 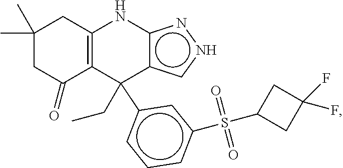 | 944019 |
| 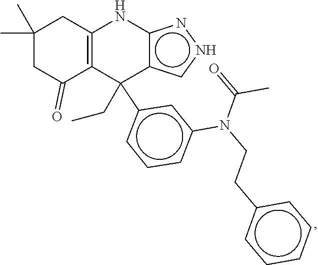 | 912064 |
| 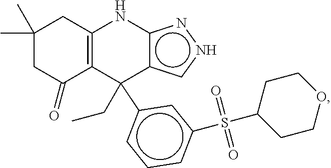 | 927344 |
| 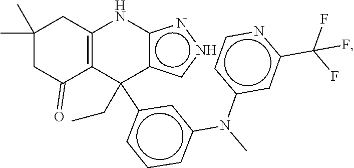 | 913254 |
| 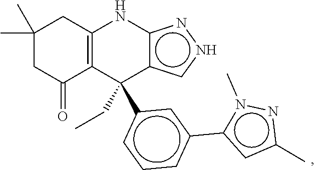 | 946097 |
| 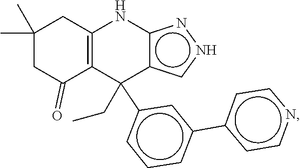 | 791271 |
| 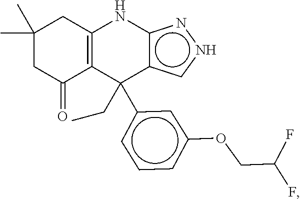 | 948793 |
| 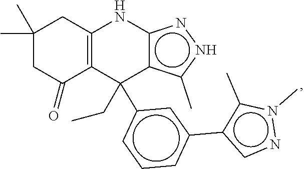 | 883993 |
| 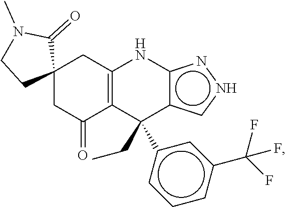 | 950329 |
| 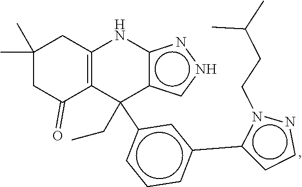 | 929401 |
| 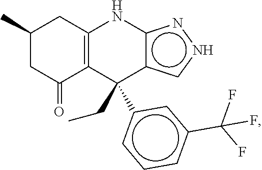 | 810451 |
| 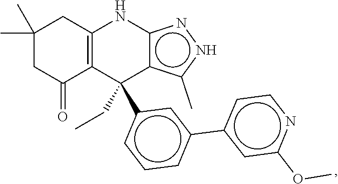 | 913053 |
| 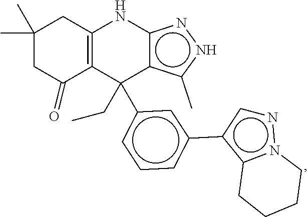 | 884002 |
| 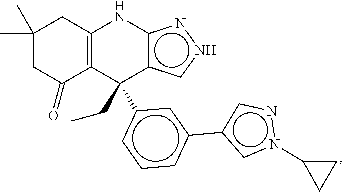 | 913028 |
| 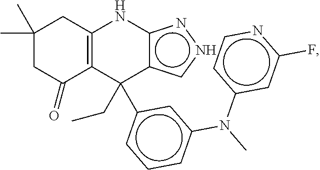 | 917146 |
| 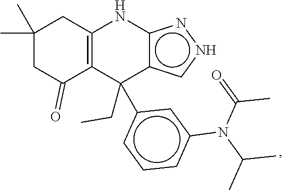 | 893305 |
| 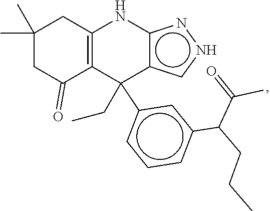 | 909205 |
| 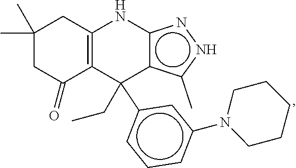 | 877311 |
| 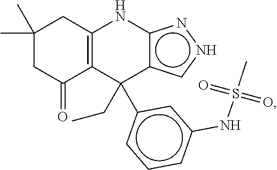 | 913253 |
| 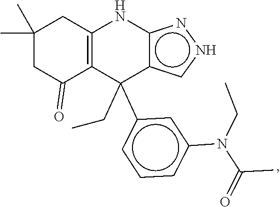 | 884109 |
| 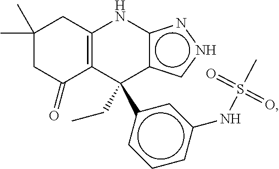 | 927348 |
| 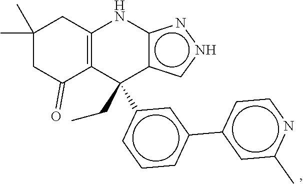 | 865169 |
| 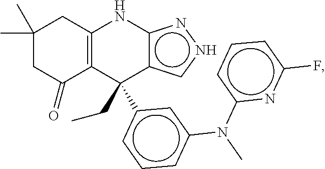 | 932636 |
| 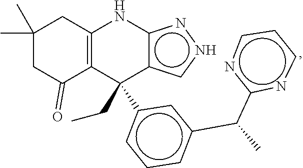 | 948058 |
| 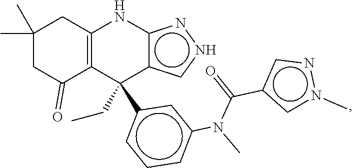 | 913310 |
| 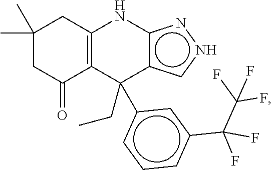 | 948615 |
| 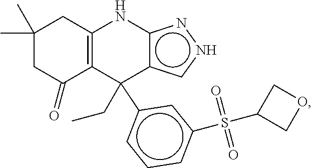 | 944017 |
| 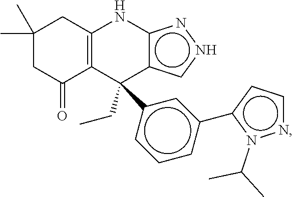 | 946091 |
| 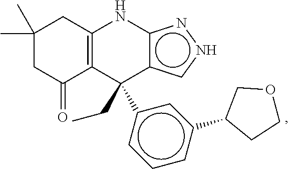 | 889556 |
| 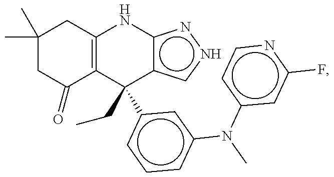 | 929444 |
| 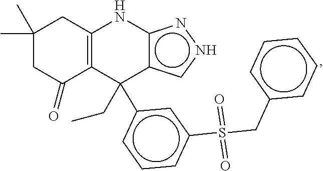 | 913258 |
| 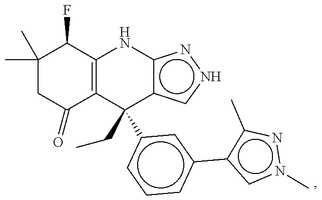 | 947650 |
| 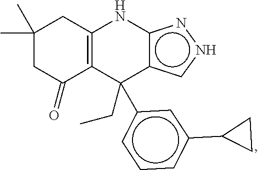 | 801316 |
| 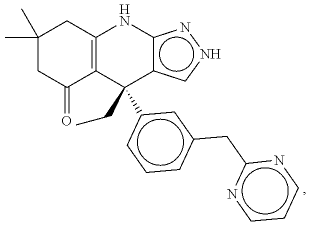 | 947498 |
| 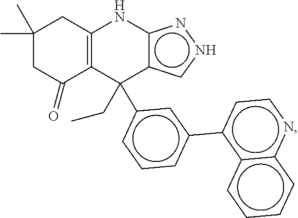 | 932631 |
| 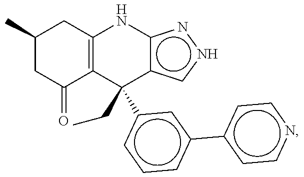 | 874317 |
| 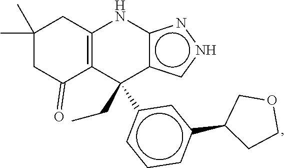 | 889557 |
| 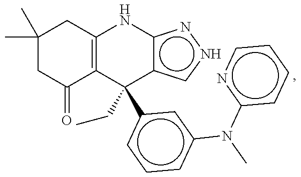 | 875568 |
| 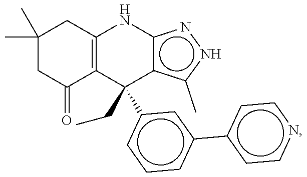 | 913015 |
| 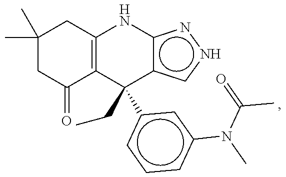 | 810358 |
| 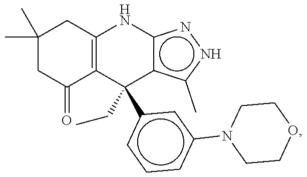 | 889581 |
| 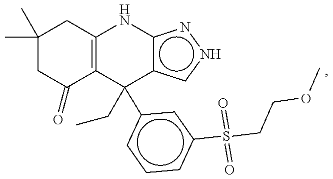 | 918271 |
| 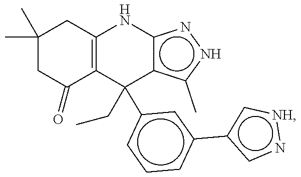 | 883994 |
| 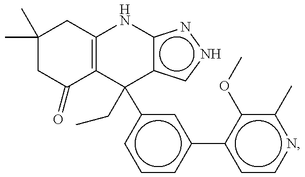 | 945458 |
| 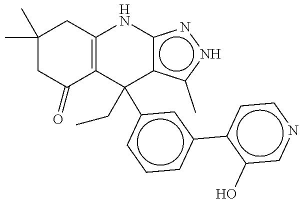 | 944024 |
| 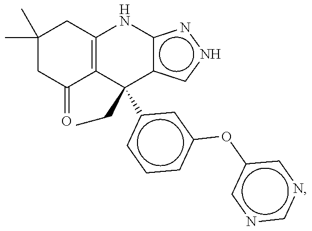 | 945933 |
| 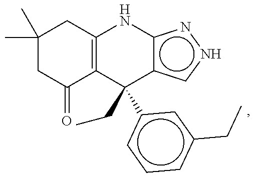 | 949401 |
| 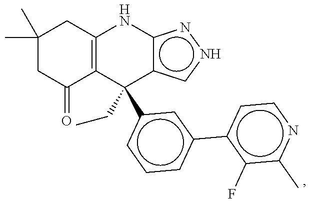 | 927347 |
| 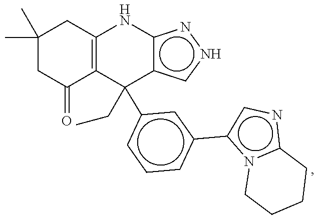 | 943535 |
| 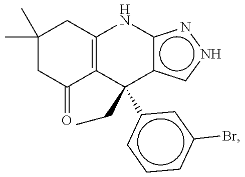 | 790733 |
| 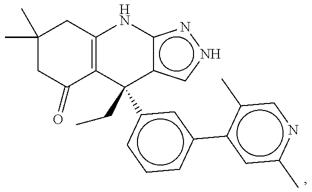 | 943514 |
| 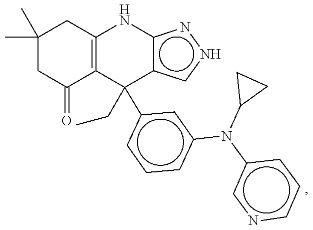 | 913274 |
| 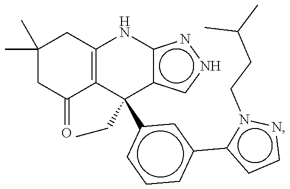 | 946095 |
| 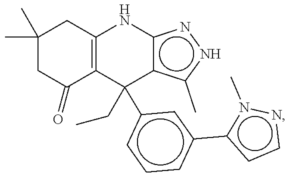 | 883992 |
| 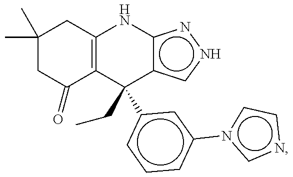 | 874531 |
| 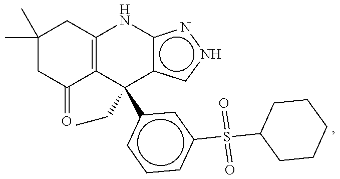 | 943541 |
| 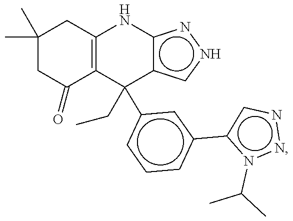 | 947397 |
| 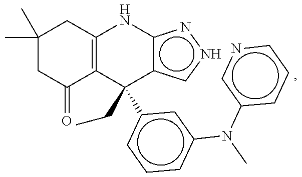 | 874298 |
| 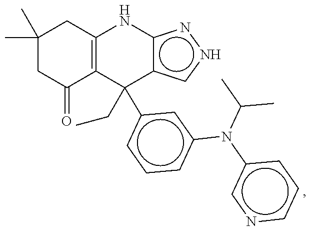 | 885344 |
| 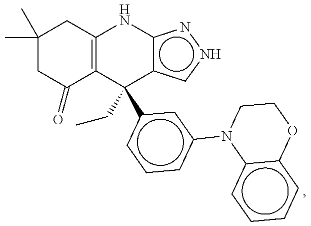 | 932655 |
| 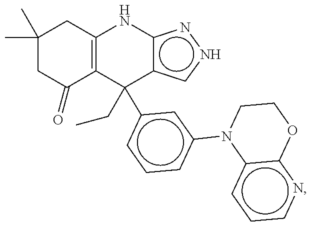 | 918279 |
| 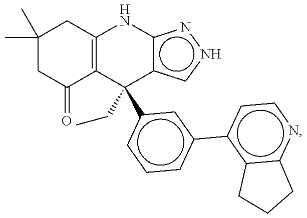 | 945606 |
| 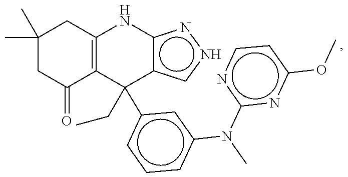 | 929387 |
| 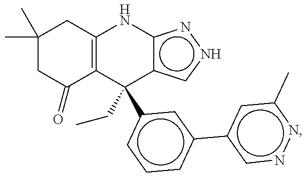 | 943994 |
| 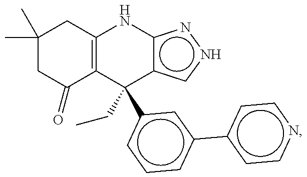 | 801413 |
| 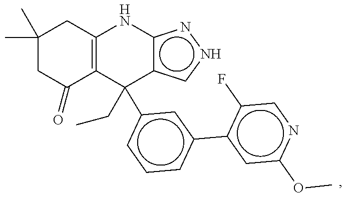 | 932630 |
| 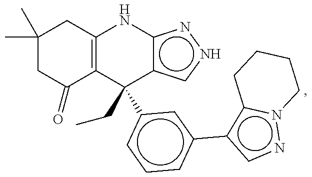 | 945543 |
| 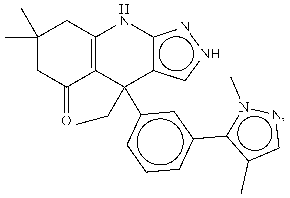 | 947491 |
| 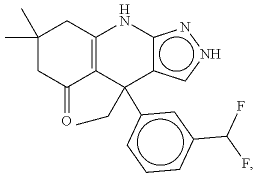 | 819564 |
| 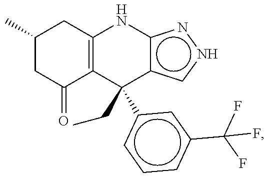 | 810450 |
| 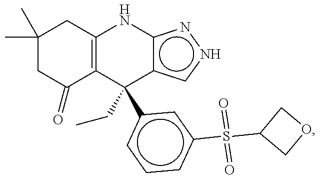 | 945998 |
| 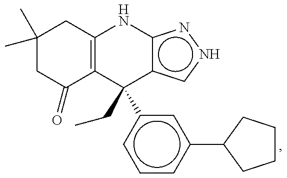 | 949397 |
| 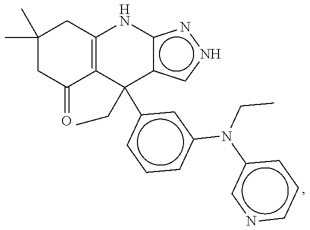 | 885331 |
| 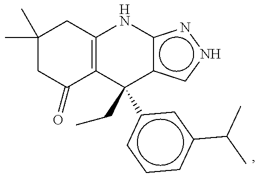 | 810373 |
| 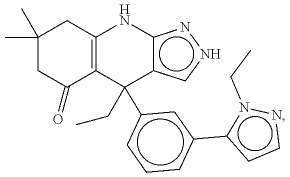 | 929398 |
| 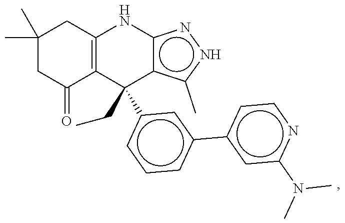 | 913021 |
| 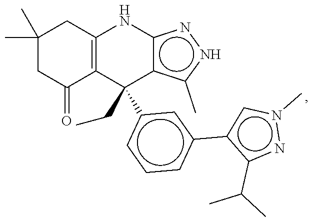 | 913029 |
| 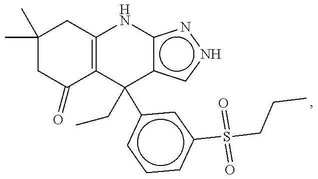 | 917131 |
| 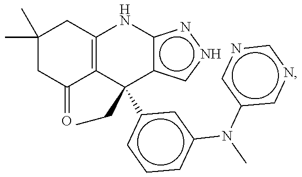 | 915400 |
| 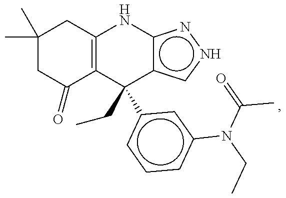 | 909214 |
| 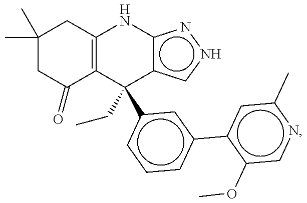 | 945995 |
| 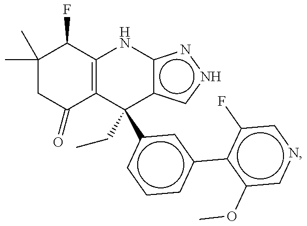 | 948547 |
| 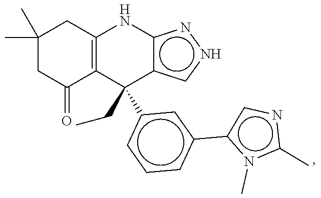 | 945613 |
| 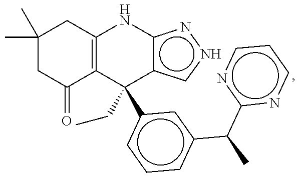 | 948060 |
| 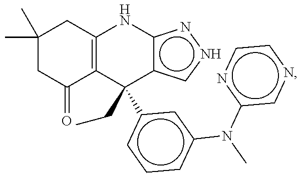 | 915316 |
| 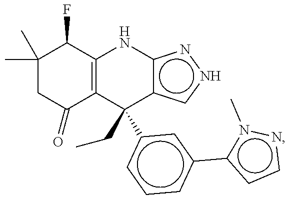 | 957507 |
| 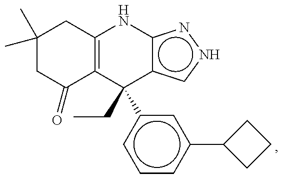 | 949521 |
| 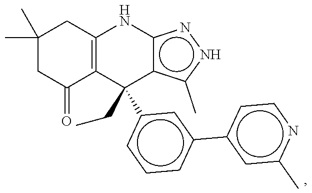 | 913018 |
| 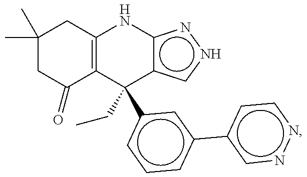 | 929454 |
| 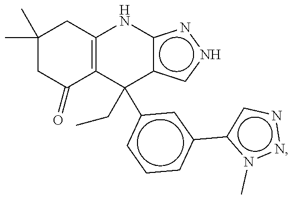 | 947398 |
| 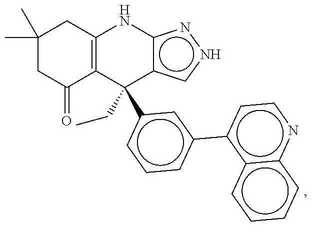 | 944031 |
| 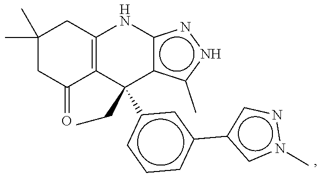 | 913031 |
| 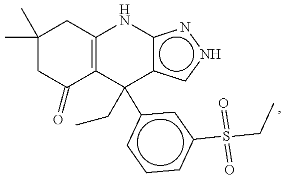 | 889579 |
| 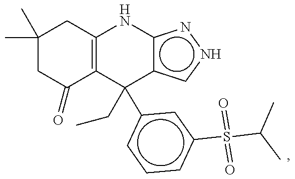 | 912169 |
| 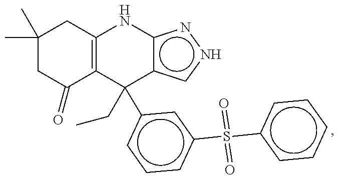 | 913123 |
| 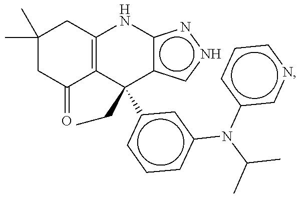 | 917086 |
| 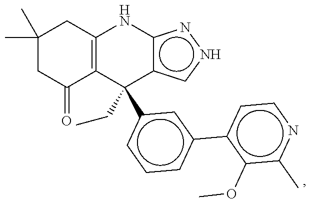 | 945997 |
| 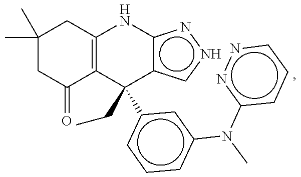 | 911975 |
| 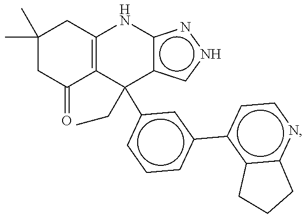 | 943534 |
| 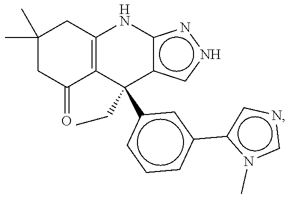 | 945605 |
| 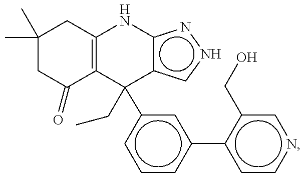 | 943539 |
| 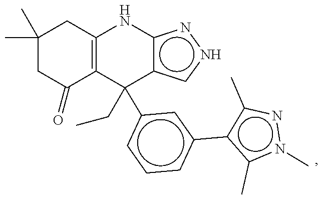 | 883851 |
| 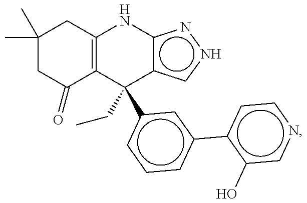 | 945724 |
| 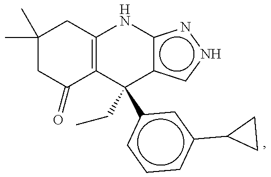 | 811557 |
| 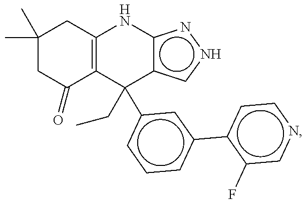 | 883986 |
| 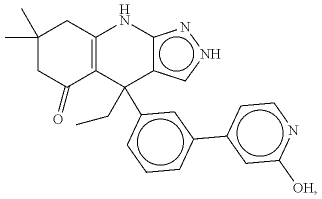 | 913352 |
| 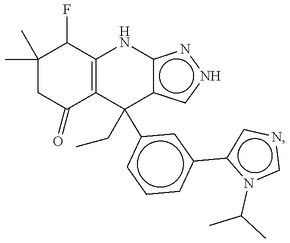 | 963155 |
| 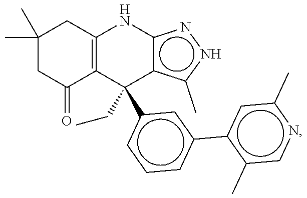 | 913050 |
| 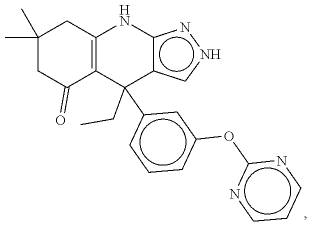 | 947400 |
| 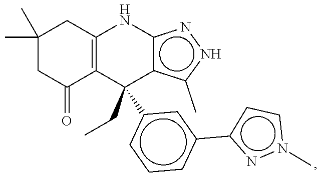 | 913030 |
| 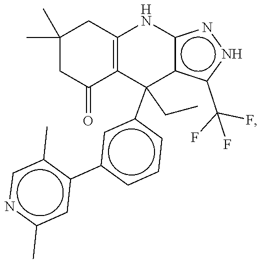 | 945530 |
| 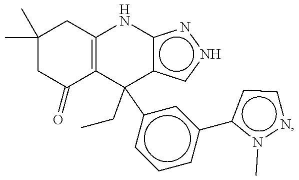 | 883852 |
| 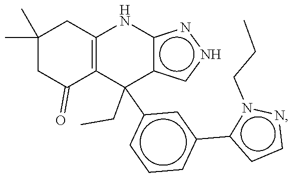 | 929400 |
| 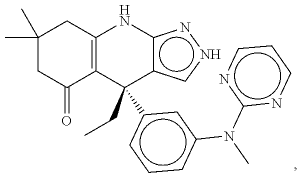 | 911980 |
| 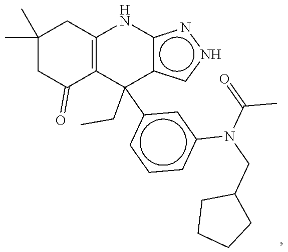 | 912166 |
| 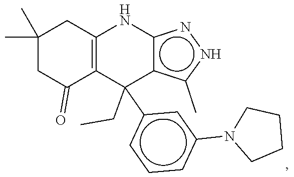 | 874314 |
| 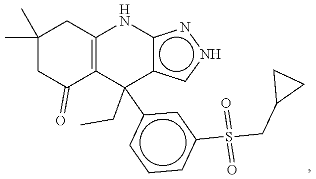 | 913257 |
| 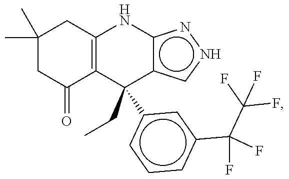 | 949103 |
| 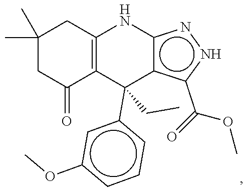 | 901479 |
| 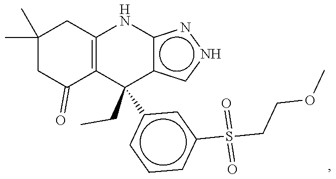 | 943542 |
| 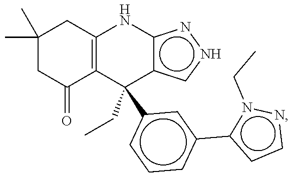 | 946089 |
| 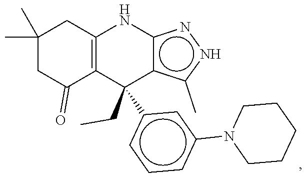 | 884116 |
| 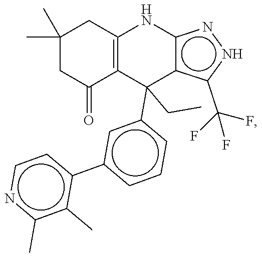 | 945529 |
| 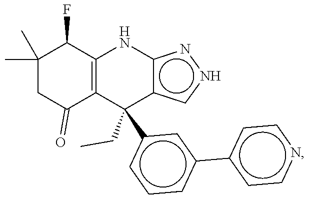 | 959343 |
| 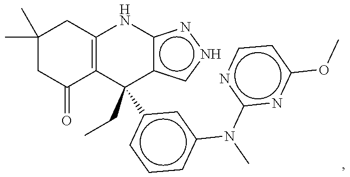 | 947336 |
| 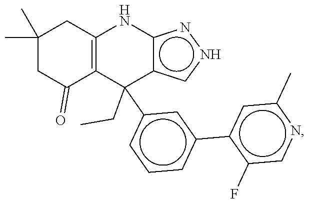 | 917132 |
| 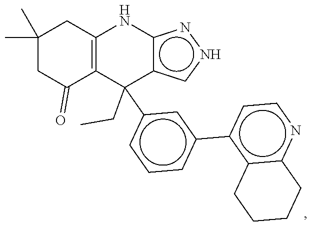 | 932625 |
| 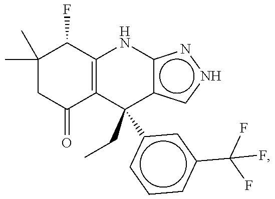 | 837646 |
| 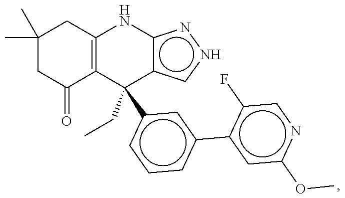 | 944032 |
| 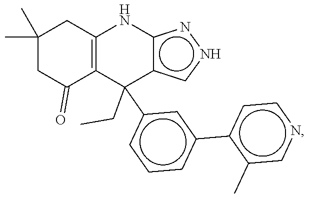 | 913351 |
| 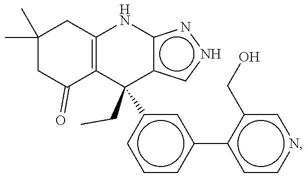 | 945469 |
| 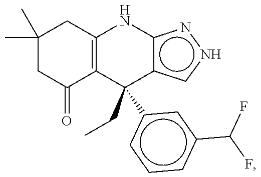 | 826717 |
| 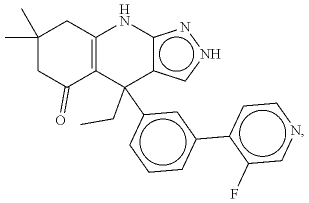 | 913349 |
| 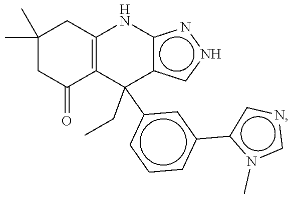 | 943533 |
| 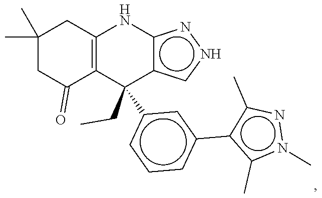 | 911990 |
| 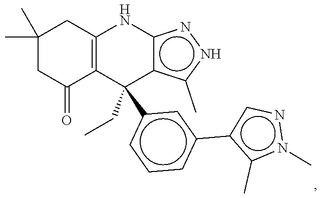 | 913045 |
| 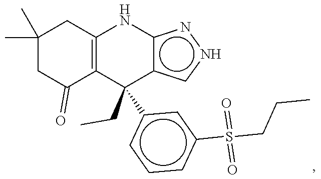 | 929447 |
| 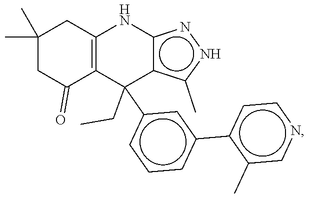 | 883991 |
| 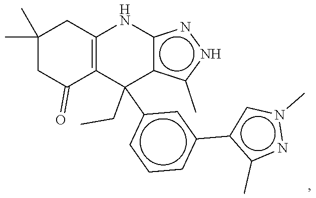 | 884003 |
| 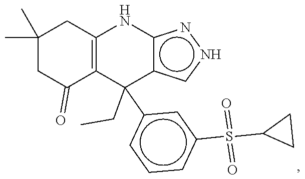 | 909204 |
| 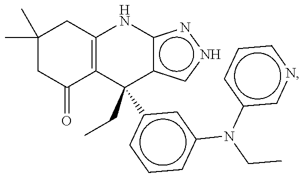 | 917088 |
| 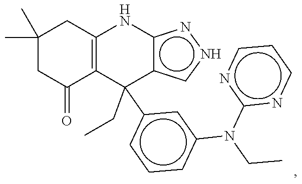 | 919840 |
| 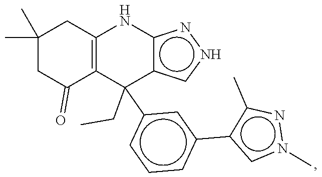 | 926840 |
| 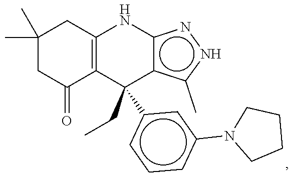 | 883874 |
| 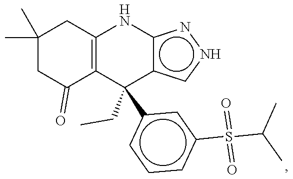 | 913135 |
| 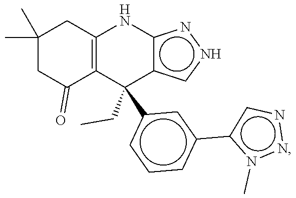 | 947802 |
| 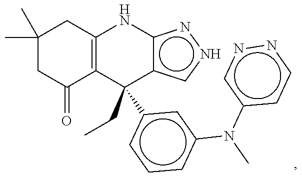 | 943492 |
| 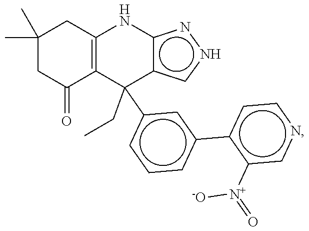 | 947631 |
| 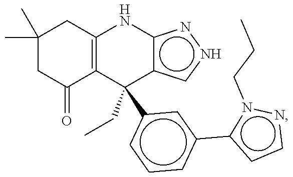 | 946093 |
| 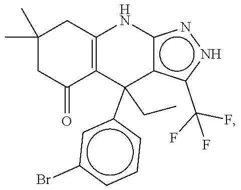 | 801324 |
| 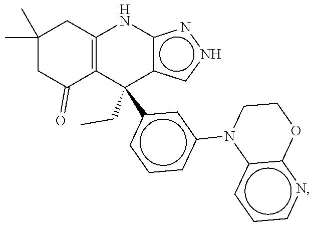 | 932663 |
| 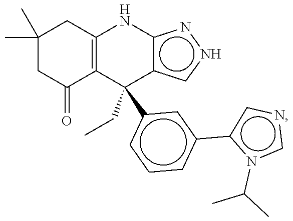 | 945725 |
| 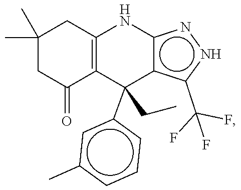 | 883873 |
| 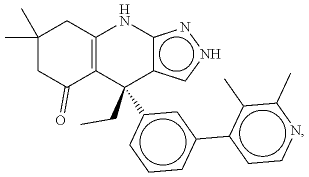 | 943516 |
| 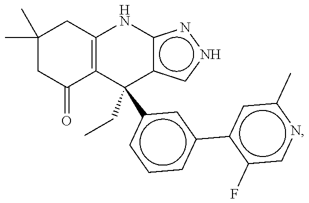 | 927346 |
| 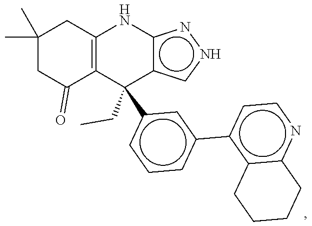 | 944033 |
| 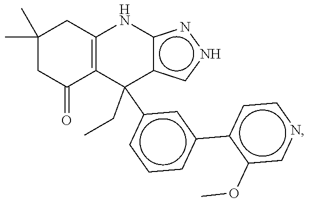 | 932627 |
| 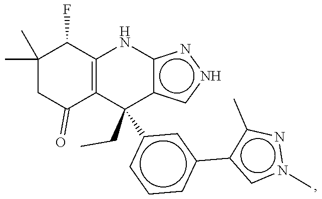 | 947651 |
| 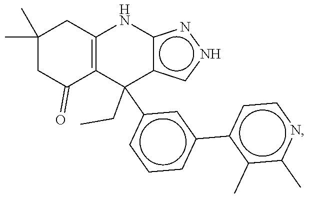 | 913347 |
| 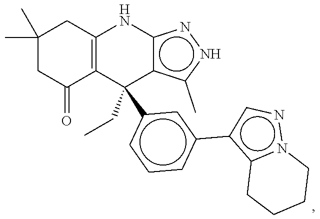 | 913036 |
| 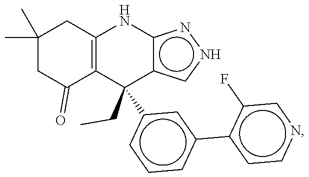 | 943518 |
| 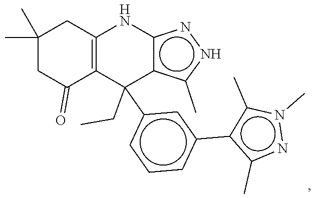 | 883995 |
| 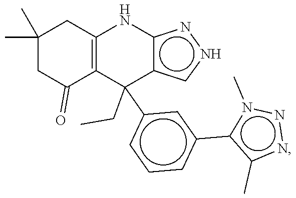 | 943996 |
| 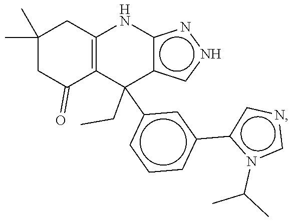 | 944026 |
| 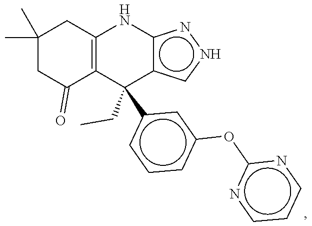 | 947808 |
| 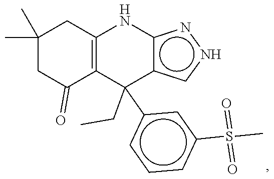 | 819561 |
| 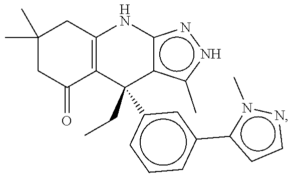 | 913023 |
| 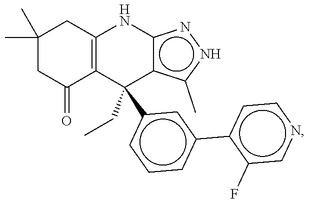 | 913052 |
| 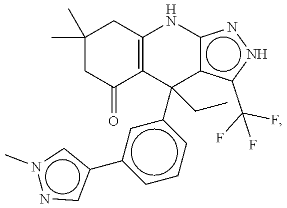 | 801445 |
| 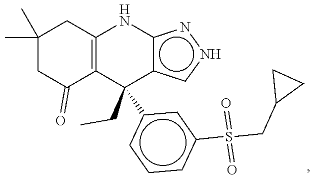 | 917150 |
| 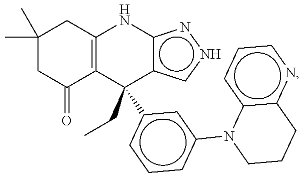 | 917076 |
| 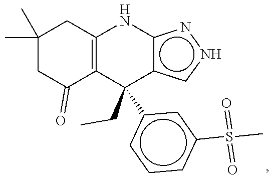 | 826718 |
| 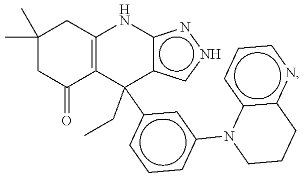 | 889639 |
| 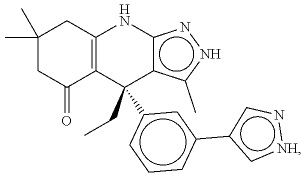 | 913025 |
| 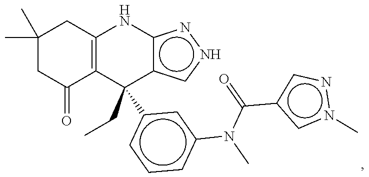 | 913311 |
| 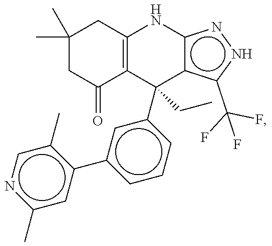 | 947363 |
| 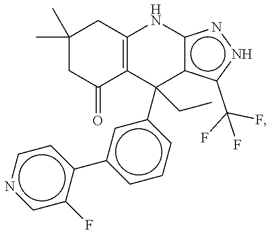 | 945528 |
| 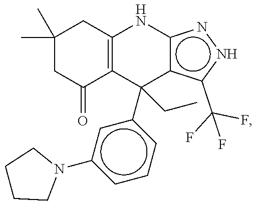 | 810506 |
| 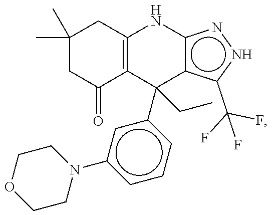 | 810507 |
| 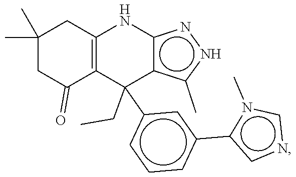 | 884001 |
| 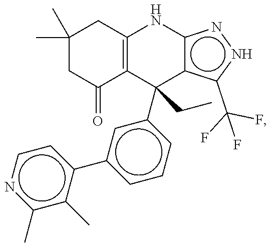 | 947329 |
| 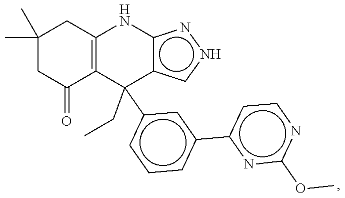 | 948092 |
| 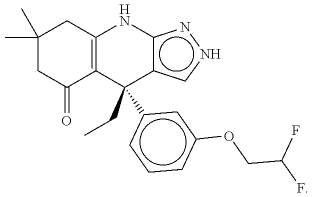 | 949104 |

106. The compound of claim 83, or a pharmaceutically acceptable salt, solvate, hydrate, polymorph, co-crystal, tautomer, stereoisomer, isotopically labeled compound, or prodrug thereof, wherein the compound is any one of the formulae shown in Table 7A:
TABLE 7A

| 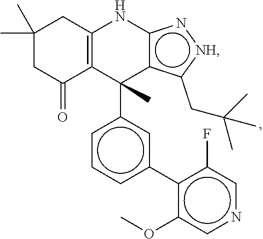 | 108954 |
|---|---|
| 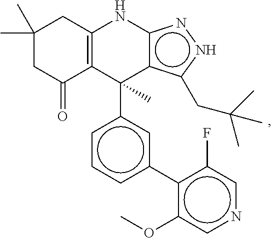 | 108956 |
| 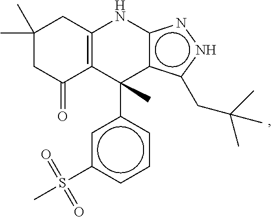 | 114687 |
| 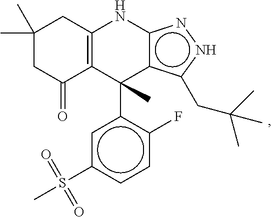 | 125976 |
| 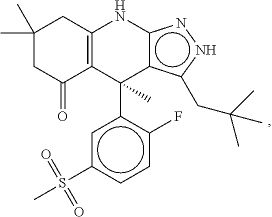 | 125977 |
| 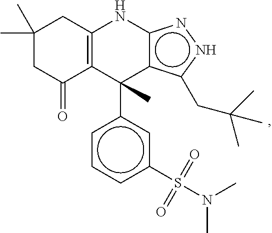 | 126012 |
| 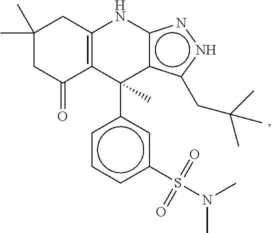 | 126013 |
| 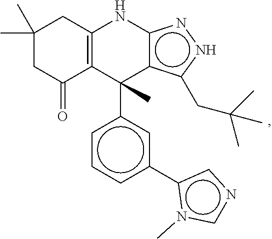 | 126014 |
| 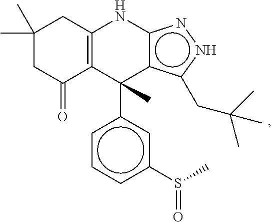 | 126190 |
| 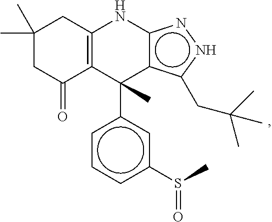 | 126191 |
| 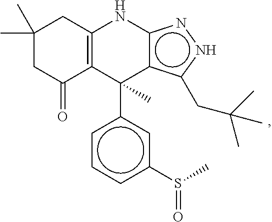 | 126192 |
| 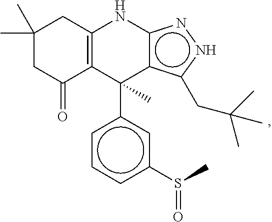 | 126193 |
| 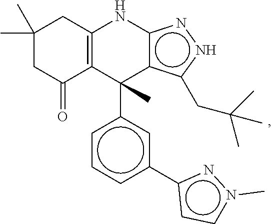 | 126366 |
| 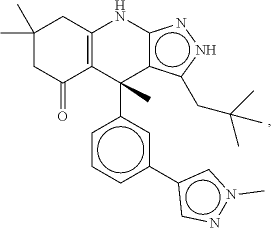 | 126393 |
| 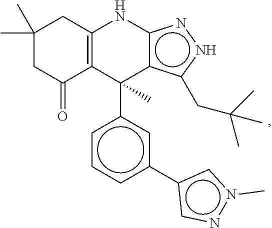 | 126394 |
| 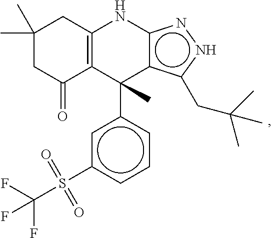 | 126395 |
| 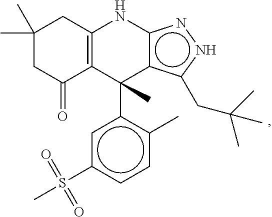 | 126397 |
| 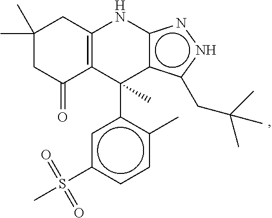 | 126398 |
| 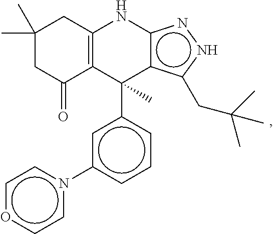 | 126399 |
| or | |
| 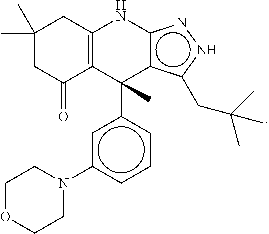 | 126400 |

107. The compound of claim 83, or a pharmaceutically acceptable salt, solvate, hydrate, polymorph, co-crystal, tautomer, stereoisomer, isotopically labeled compound, or prodrug thereof, wherein the compound is any one of the formulae shown in Table 8:
TABLE 8

| | |
|---|---|
| 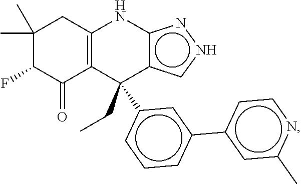 | 945485 |
| 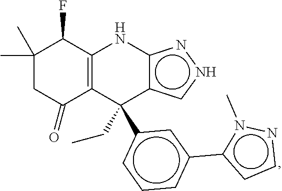 | 957508 |
| 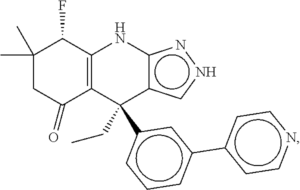 | 959344 |
| 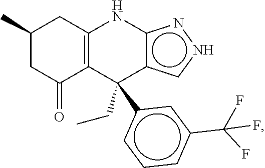 | 810453 |
| 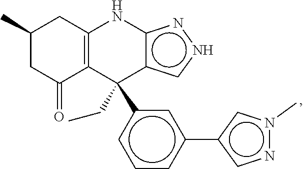 | 823726 |
| 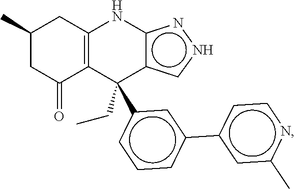 | 932637 |
| 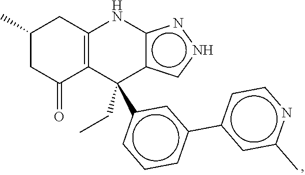 | 932638 |
| 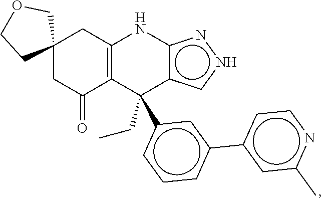 | 947411 |
| 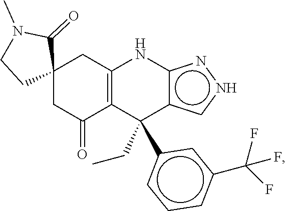 | 950325 |
| 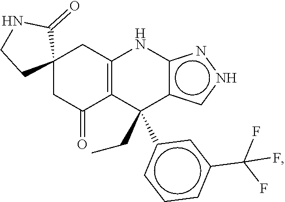 | 950332 |
| 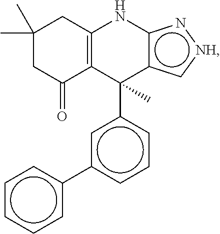 | 801366 |
| 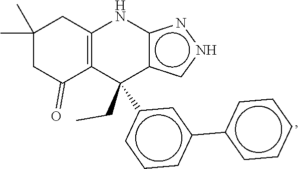 | 801369 |
| 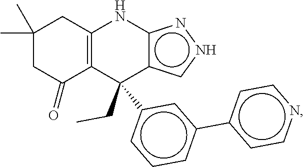 | 801412 |
| 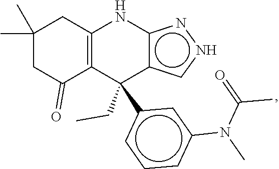 | 810359 |
| 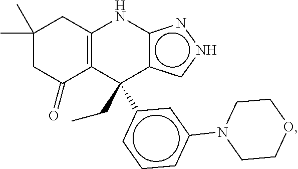 | 810364 |
| 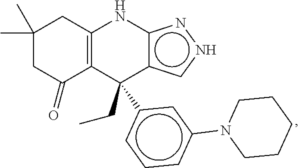 | 810371 |
| 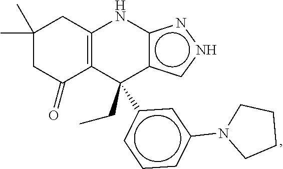 | 810509 |
| 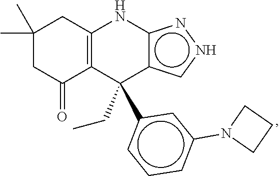 | 889632 |
| 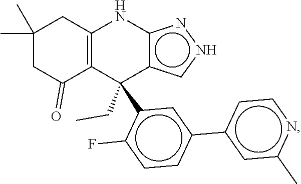 | 909213 |
| 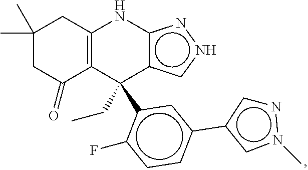 | 909223 |
| 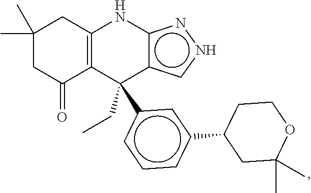 | 912091 |
| 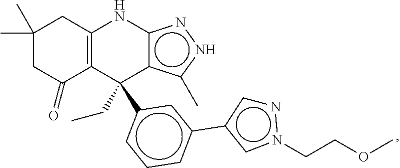 | 913042 |
| 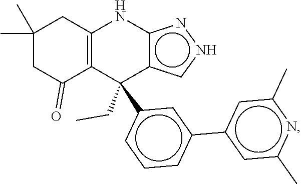 | 913223 |
| 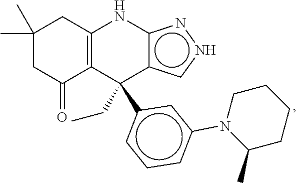 | 917210 |
| 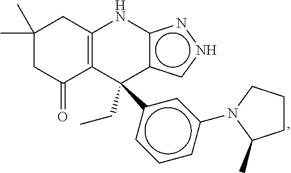 | 918322 |
| 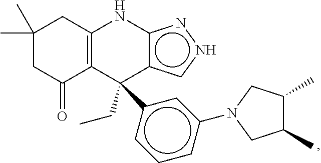 | 924520 |
| 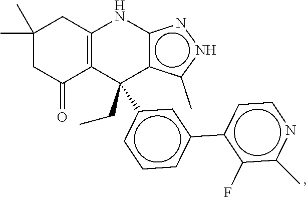 | 927340 |
| 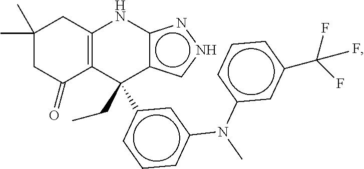 | 927345 |
| 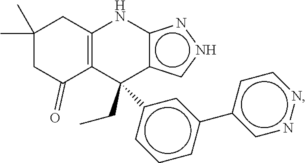 | 929443 |
| 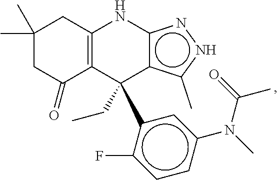 | 929451 |
| 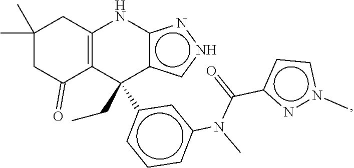 | 929455 |
| 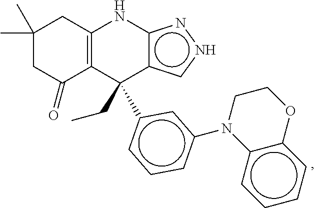 | 932635 |
| 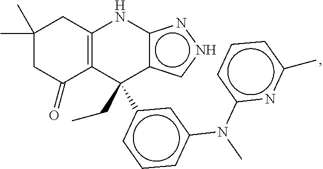 | 932644 |
| 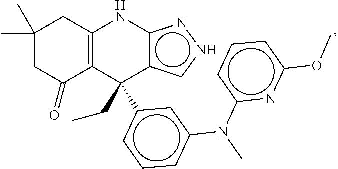 | 932646 |
| 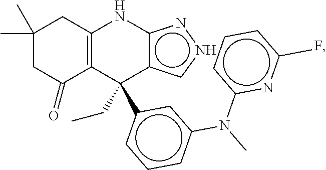 | 932656 |
| 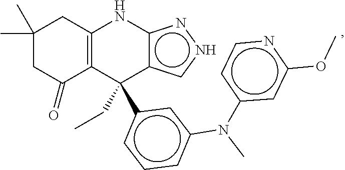 | 932662 |
| 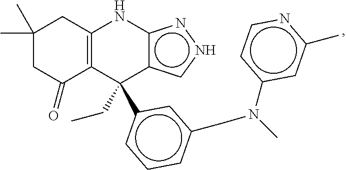 | 932665 |
| 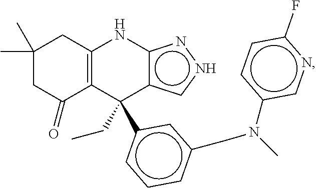 | 932667 |
| 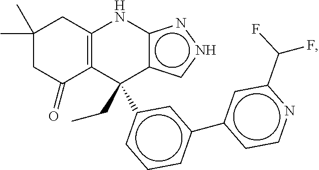 | 943915 |
| 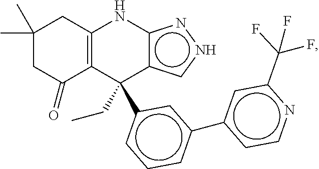 | 943989 |
| 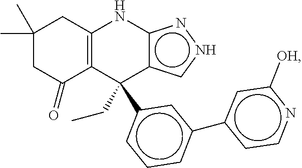 | 943993 |
| 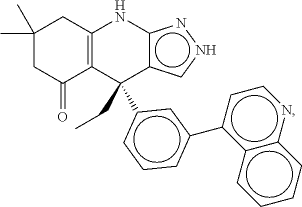 | 944034 |
| 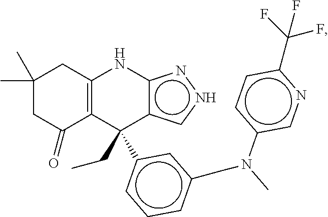 | 945484 |
| 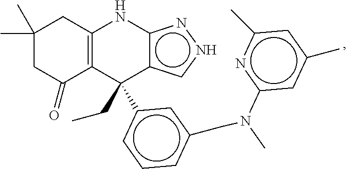 | 945615 |
| 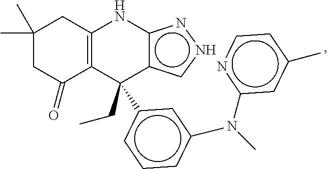 | 945616 |
| 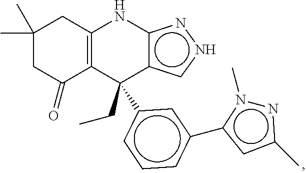 | 946000 |
| 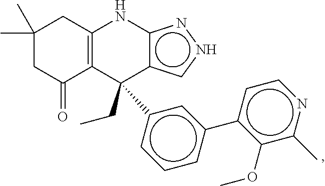 | 946002 |
| 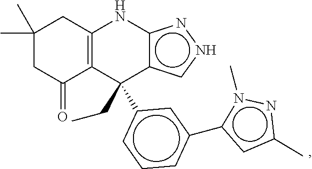 | 946098 |
| 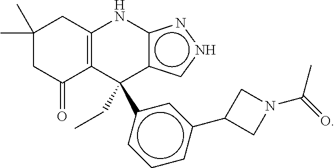 | 949132 |

108. The compound of claim 83, or a pharmaceutically acceptable salt, solvate, hydrate, polymorph, co-crystal, tautomer, stereoisomer, isotopically labeled compound, or prodrug thereof, wherein the compound is any one of the formulae shown in Table 8A:
TABLE 8A

| | |
|---|---|
| 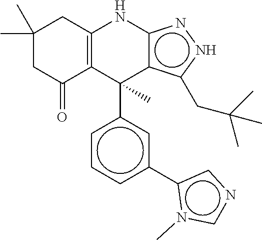 | 126015 |
| 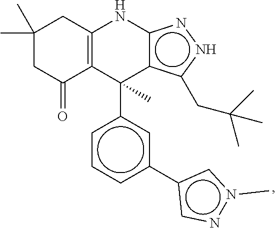 | 126367 |

or

| | |
|---|---|
| 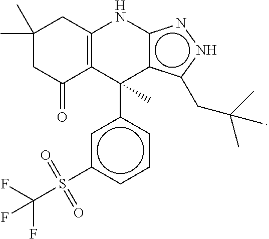 | 126396 |

109. The compound of claim 83, or a pharmaceutically acceptable salt, solvate, hydrate, polymorph, co-crystal, tautomer, stereoisomer, isotopically labeled compound, or prodrug thereof, wherein the compound is not a compound of claim 107 or 108.

110. A compound, or a pharmaceutically acceptable salt, solvate, hydrate, polymorph, co-crystal, tautomer, stereoisomer, isotopically labeled compound, or prodrug thereof, wherein the compound is of Formula V-A or V-B: 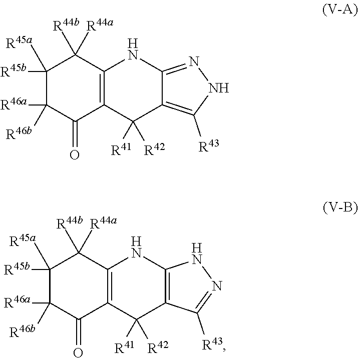
wherein:
R⁴¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R⁴² is optionally substituted alkyl, optionally substituted alkenyl, or optionally substituted alkynyl;
or R⁴¹ and R⁴² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
R⁴³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{E}, —SCN, —SR^{E}, —SSR^{E}, —N₃, —NO, —N(R^{E})₂, —NO₂, —C(═O)R^{E}, —C(═O)OR^{E}, —C(═O)SR^{E}, —C(═O)N(R^{E})₂, —C(═NR^{E})R^{E}, —C(═NR^{E})OR^{E}, —C(═NR^{E})SR^{E}, —C(═NR^{E})N(R^{E})₂, —S(═O)R^{E}, —S(═O)OR^{E}, —S(═O)SR^{E}, —S(═O)N(R^{E})₂, —S(═O)₂R^{E}, —S(═O)₂OR^{E}, —S(═O)₂SR^{E}, —S(═O)₂N(R^{E})₂, —OC(═O)R^{E}, —OC(═O)OR^{E}, —OC(═O)SR^{E}, —OC(═O)N(R^{E}), —OC(═NR^{E})R^{E}, —OC(═NR^{E})OR^{E}, —OC(═NR^{E})SR^{E}, —OC(═NR^{E})N(R^{E})₂, —OS(═O)R^{E}, —OS(═O)OR^{E}, —OS(═O)SR^{E}, —OS(═O)N(R^{E})₂, —OS(═O)₂R^{E}, —OS(═O)₂OR^{E}, —OS(═O)₂SR^{E}, —OS(═O)₂N(R^{E})₂, —ON(R^{E})₂, —SC(═O)R^{E}, —SC(═O)OR^{E}, —SC(═O)SR^{E}, —SC(═O)N(R^{E})₂, —SC(═NR^{E})R^{E}, —SC(═NR^{E})OR^{E}, —SC(═NR^{E})SR^{E}, —SC(═NR^{E})N(R^{E})₂, —NR^{E}C(═O)R^{E}, —NR^{E}C(═O)OR^{E}, —NR^{E}C(═O)SR^{E}, —NR^{E}C(═O)N(R^{E})₂, —NR^{E}C(═NR^{E})R^{E}, —NR^{E}C(═NR^{E})OR^{E}, —NR^{E}C(═NR^{E})SR^{E}, —NR^{E}C(═NR^{E})N(R^{E})₂, —NR^{E}S(═O)R^{E}, —NR^{E}S(═O)OR^{E}, —NR^{E}S(═O)SR^{E}, —NR^{E}S(═O)N(R^{E})₂, —NR^{E}S(═O)₂R^{E}, —NR^{E}S(═O)₂OR^{E}, —NR^{E}S(═O)₂SR^{E}, —NR^{E}S(═O)₂N(R^{E})₂, —Si(R^{E})₃, —Si(R^{E})₂OR^{E}, —Si(R^{F})(OR^{F})₂, —Si(OR^{E})₃, —OSi(R^{E})₃, —OSi(R^{E})₂OR^{E}, —OSi(R^{E})(OR^{E})₂, or —OSi(OR^{E})₃;
each of R^{44a}, R^{44b}, R^{45a}, R^{45b}, R^{46a}, and R^{46b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or —OR^{E}; and
each instance of R^{E} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{E} attached to the same intervening atom are joined together with the intervening atom to form an optionally substituted, monocyclic, heterocyclic or heteroaryl ring;
or R^{44a} and R^{44b} are taken together with their intervening atom to form an optionally substituted, monocyclic, carbocyclic or heterocyclic ring;
or R^{44b} and R^{45a} are taken together with their intervening atom to form an optionally substituted, monocyclic, carbocyclic or heterocyclic ring;
or R^{45a} and R^{45b} are taken together with their intervening atom to form an optionally substituted, monocyclic, carbocyclic or heterocyclic ring;
or R^{45b} and R^{46a} are taken together with their intervening atom to form an optionally substituted, monocyclic, carbocyclic or heterocyclic ring;
or R^{46a} and R^{46b} are taken together with their intervening atom to form an optionally substituted, monocyclic, carbocyclic or heterocyclic ring;
provided that at least one of R^{44a}, R^{44b}, R^{46a}, and R^{46b} is not hydrogen.

111. The compound of claim 110, or a pharmaceutically acceptable salt, solvate, hydrate, polymorph, co-crystal, tautomer, stereoisomer, isotopically labeled compound, or prodrug thereof, wherein each of R^{46a} and R^{4b}h is independently hydrogen, halogen, optionally substituted alkyl, or —OR^{E}.

112. The compound of claim 110 or 111, or a pharmaceutically acceptable salt, solvate, hydrate, polymorph, co-crystal, tautomer, stereoisomer, isotopically labeled compound, or prodrug thereof, wherein one of R^{46a} and R^{46b} is hydrogen.

113. The compound of any one of claims 110-112, or a pharmaceutically acceptable salt, solvate, hydrate, polymorph, co-crystal, tautomer, stereoisomer, isotopically labeled compound, or prodrug thereof, wherein each of R^{45a} and R^{45b} is independently hydrogen, halogen, or optionally substituted alkyl.

114. The compound of claim 113, or a pharmaceutically acceptable salt, solvate, hydrate, polymorph, co-crystal, tautomer, stereoisomer, isotopically labeled compound, or prodrug thereof, wherein R^{45a} and R^{45b} are —CH₃.

115. The compound of any one of claims 110-114, or a pharmaceutically acceptable salt, solvate, hydrate, polymorph, co-crystal, tautomer, stereoisomer, isotopically labeled compound, or prodrug thereof, wherein each of R^{44a} and R^{44b} is independently hydrogen, halogen, or optionally substituted alkyl.

116. The compound of claim 115, or a pharmaceutically acceptable salt, solvate, hydrate, polymorph, co-crystal, tautomer, stereoisomer, isotopically labeled compound, or prodrug thereof, wherein one of R^{44a} and R^{44b} is hydrogen.

117. The compound of any one of claims 110-116, or a pharmaceutically acceptable salt, solvate, hydrate, polymorph, co-crystal, tautomer, stereoisomer, isotopically labeled compound, or prodrug thereof, wherein R⁴¹ is optionally substituted aryl.

118. The compound of claim 117, or a pharmaceutically acceptable salt, solvate, hydrate, polymorph, co-crystal, tautomer, stereoisomer, isotopically labeled compound, or prodrug thereof, wherein R⁴¹ is 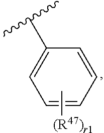
wherein:
each instance of R⁴⁷ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(O)R^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —OSi(R^{D})₃, —OSi(R^{D})₂OR^{D}, —OSi(R^{D})(OR^{D})₂, or —OSi(OR^{D})₃;
r1 is 0, 1, 2, 3, 4, or 5.

119. The compound of claim 118, or a pharmaceutically acceptable salt, solvate, hydrate, thereof, wherein R⁴¹ is

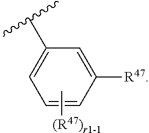

120. The compound of claim 118 or 119, or a pharmaceutically acceptable salt, solvate, hydrate, polymorph, co-crystal, tautomer, stereoisomer, isotopically labeled compound, or prodrug thereof, wherein r1 is 1.

121. The compound of any one of claims 110-120, or a pharmaceutically acceptable salt, solvate, hydrate, polymorph, co-crystal, tautomer, stereoisomer, isotopically labeled compound, or prodrug thereof, wherein R⁴² is optionally substituted alkyl.

122. The compound of claim 121, or a pharmaceutically acceptable salt, solvate, hydrate, polymorph, co-crystal, tautomer, stereoisomer, isotopically labeled compound, or prodrug thereof, wherein R⁴² is unsubstituted C₁-C₄ alkyl.

123. The compound of any one of claims 110-116, or a pharmaceutically acceptable salt, solvate, hydrate, polymorph, co-crystal, tautomer, stereoisomer, isotopically labeled compound, or prodrug thereof, wherein R⁴¹ and R⁴² are taken together with their intervening atom to form an optionally substituted, monocyclic, carbocyclic ring.

124. The compound of any one of claims 110-116, or a pharmaceutically acceptable salt, solvate, hydrate, polymorph, co-crystal, tautomer, stereoisomer, isotopically labeled compound, or prodrug thereof, wherein R⁴¹ and R⁴² are taken together with their intervening atom to form an optionally substituted, monocyclic, heterocyclic ring.

125. The compound of claim 124, or a pharmaceutically acceptable salt, solvate, hydrate, polymorph, co-crystal, tautomer, stereoisomer, isotopically labeled compound, or prodrug thereof, wherein R⁴¹ and R⁴² are taken together with their intervening atom to form an optionally substituted cyclohexyl, piperidinyl, or tetrahydropyranyl.

126. The compound of any one of claims 110-125, or a pharmaceutically acceptable salt, solvate, hydrate, polymorph, co-crystal, tautomer, stereoisomer, isotopically labeled compound, or prodrug thereof, wherein R⁴³ is hydrogen, optionally substituted C₁-C₆ alkyl, or halogen.

127. The compound of claim 126, or a pharmaceutically acceptable salt, solvate, hydrate, polymorph, co-crystal, tautomer, stereoisomer, isotopically labeled compound, or prodrug thereof, wherein R⁴³ is hydrogen, fluorine, —CH₃, —CH₂F, —CHF₂, or —CF₃.

128. The compound of claim 110, or a pharmaceutically acceptable salt, solvate, hydrate, polymorph, co-crystal, tautomer, stereoisomer, isotopically labeled compound, or prodrug thereof, wherein the compound is of the formula:

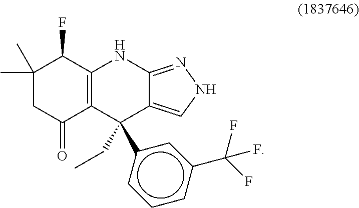

129. The compound of claim 110, or a pharmaceutically acceptable salt, solvate, hydrate, polymorph, co-crystal, tautomer, stereoisomer, isotopically labeled compound, or prodrug thereof, wherein the compound is any one of the formulae shown in Table 9:
TABLE 9 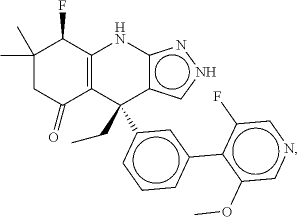
948545 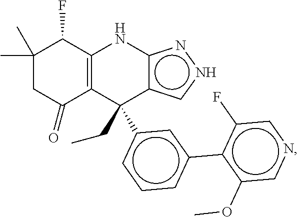
948546 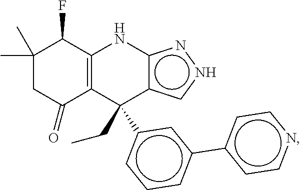
959342 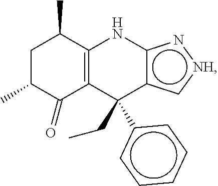
865199 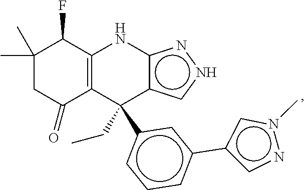
957510 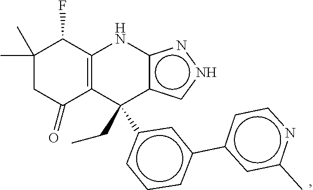
946101 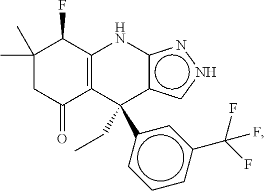
837803 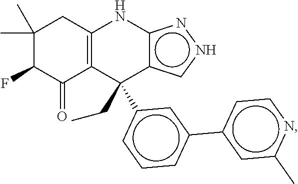
945486 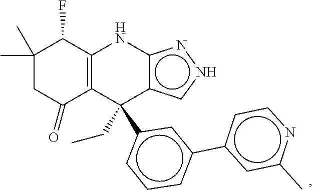
946102 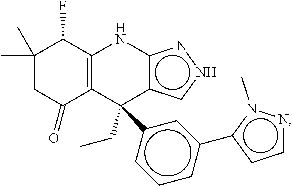
957509 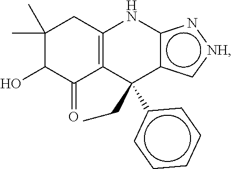
946056 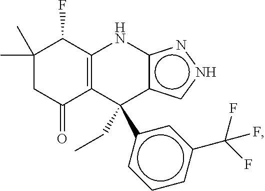
837753 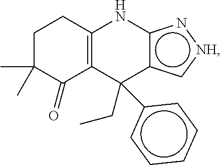
835310 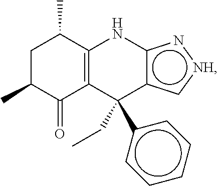
865198 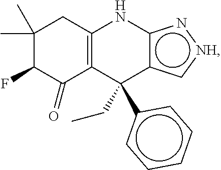
957403 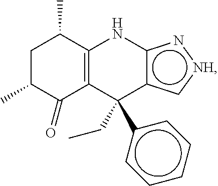
909217 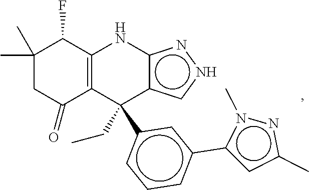
947657 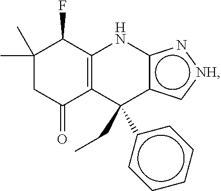
957197 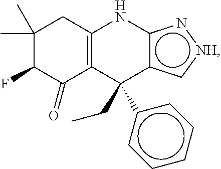
957405 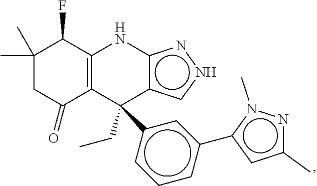
947656 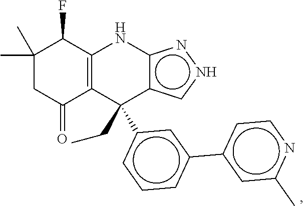
946103 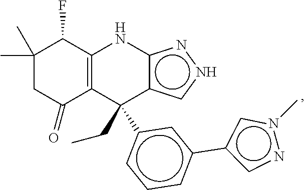
957504 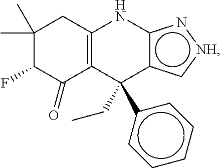
826766 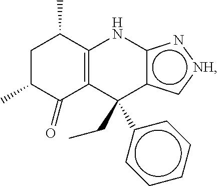
909216 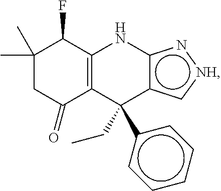
957200 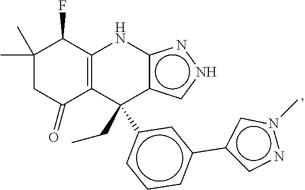
957505 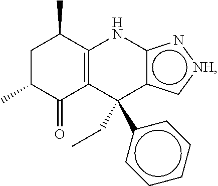
865201 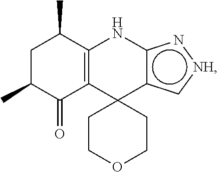
893316 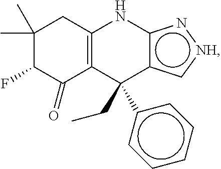
826768 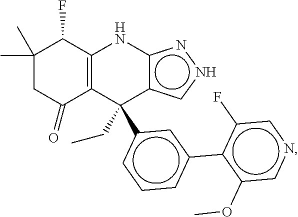
948548 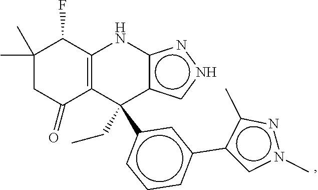
947653 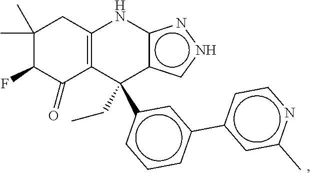
945475 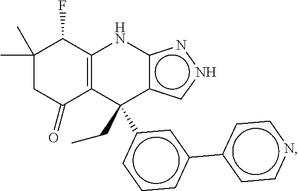
959341 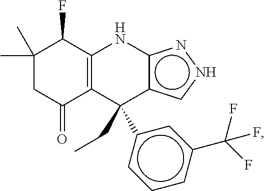
837683 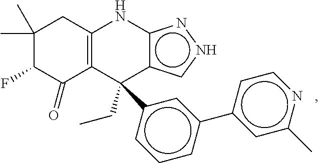
945476 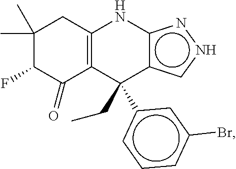
835314 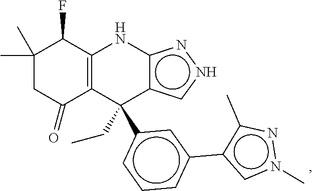
947652 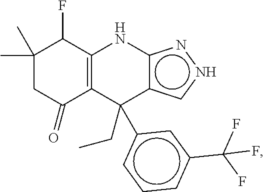
819622 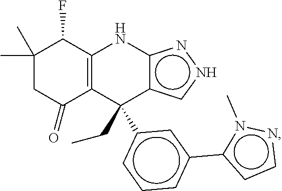
957506 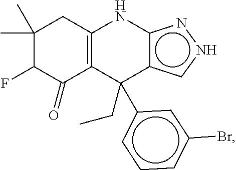
826764 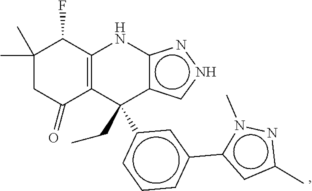
947655 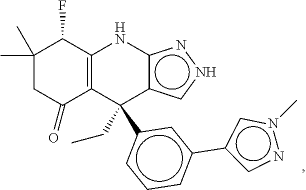
957511 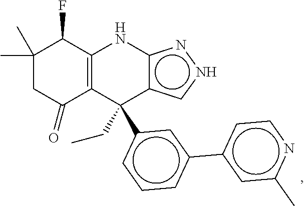
946104 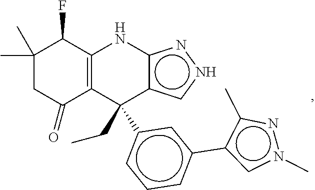
947650 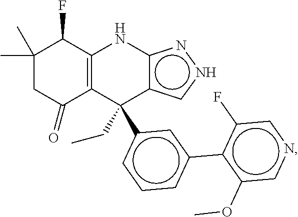
948547 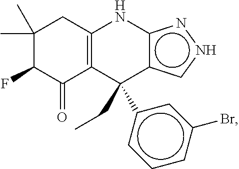
835315 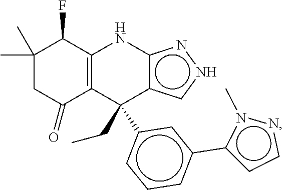
957507 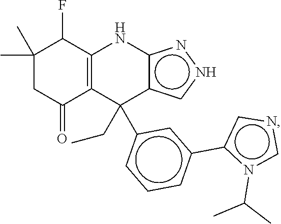
963155 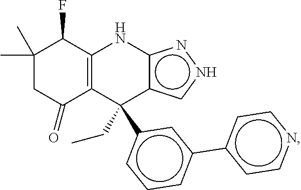
959343 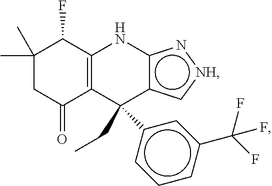
837646 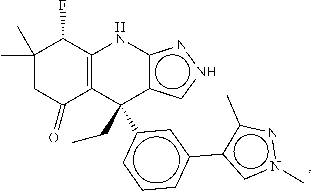
947651 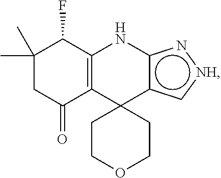
883833 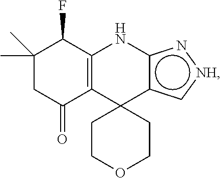
883834 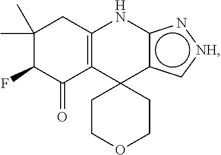
883836 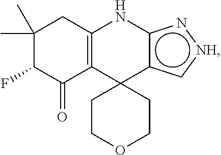
883835 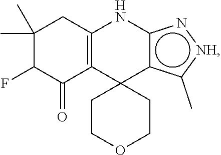
912072 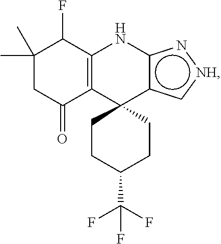
917136 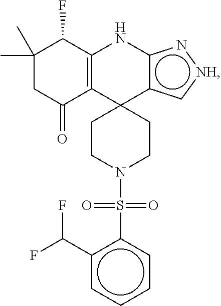
951839 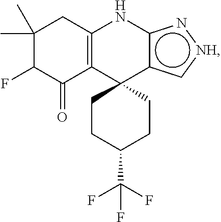
917134 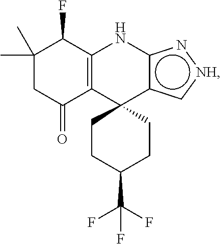
918284 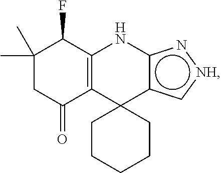
951705 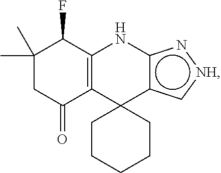
951705 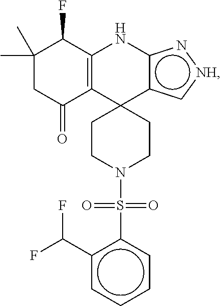
951846 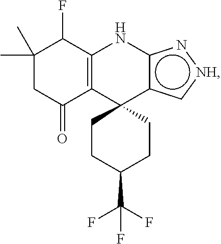
917135 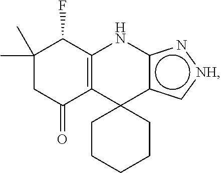
951711 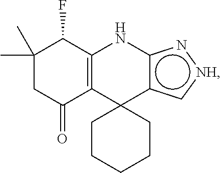
951711 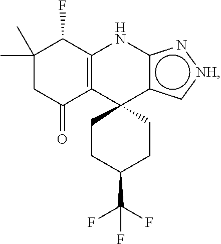
918285 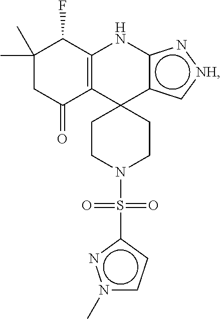
951708 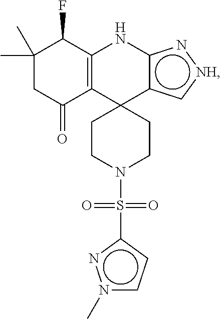
951709 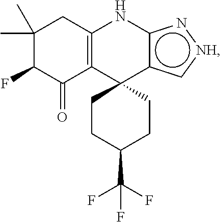
918282 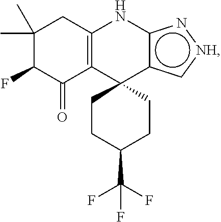
917133 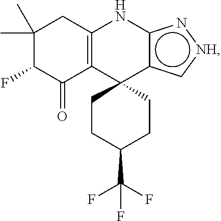
918283 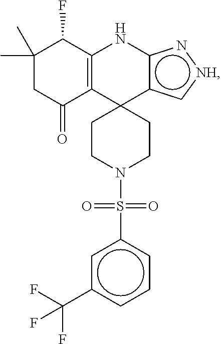
951713 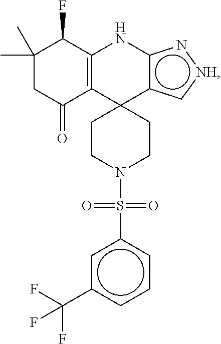
951703 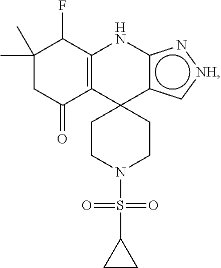
950814 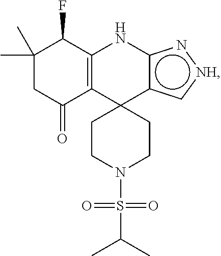
951701

130. The compound of claim 110, or a pharmaceutically acceptable salt, solvate, hydrate, polymorph, co-crystal, tautomer, stereoisomer, isotopically labeled compound, or prodrug thereof, wherein the compound is any one of the formulae shown in Table 10:
TABLE 10

| | |
|---|---|
| 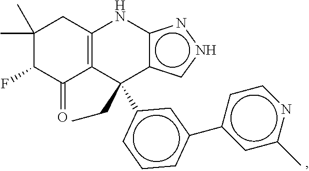 | 945485 |
| 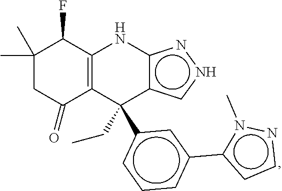 | 957508 |
| 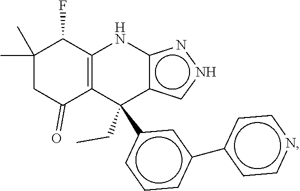 | 959344 |
| 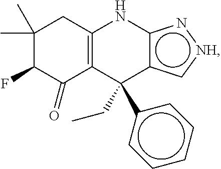 | 826767 |
| 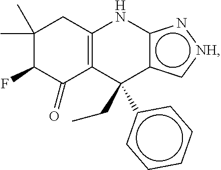 | 826769 |
| 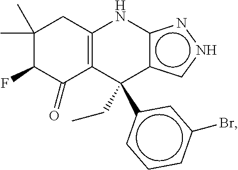 | 835312 |
| 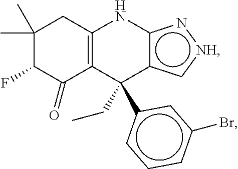 | 835313 |
| 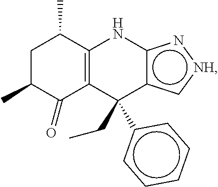 | 865202 |
| 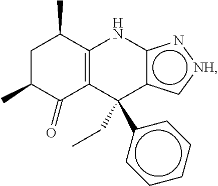 | 909218 |
| 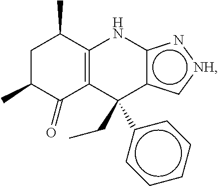 | 909219 |
| 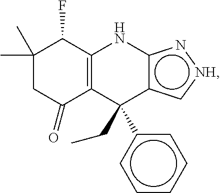 | 957196 |
| 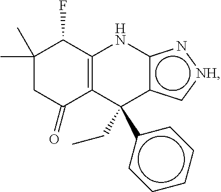 | 957199 |
| 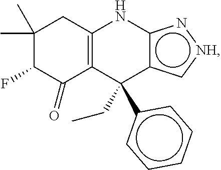 | 957404 |
| 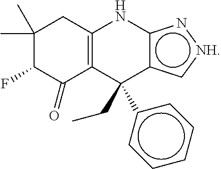 | 957406 |

131. The compound of claim 110, or a pharmaceutically acceptable salt, solvate, hydrate, polymorph, co-crystal, tautomer, stereoisomer, isotopically labeled compound, or prodrug thereof, wherein the compound is not a compound of claim 130.

132. A compound, or a pharmaceutically acceptable salt, solvate, hydrate, polymorph, co-crystal, tautomer, stereoisomer, isotopically labeled compound, or prodrug thereof, wherein the compound is of Formula VI-A or VI-B: 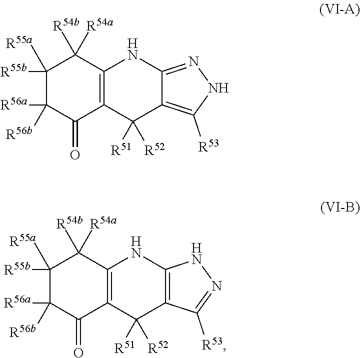
wherein:
R⁵¹ is optionally substituted alkyl, optionally substituted alkenyl, optionally substituted alkynyl, optionally substituted carbocyclyl, optionally substituted heterocyclyl, optionally substituted aryl, or optionally substituted heteroaryl;
R⁵² is optionally substituted alkyl, optionally substituted alkenyl, or optionally substituted alkynyl;
or R⁵¹ and R⁵² are taken together with their intervening atom to form an optionally substituted, monocyclic, carbocyclic or heterocyclic ring, which is optionally fused to an optionally substituted, aryl, heteroaryl, carbocyclic, or heterocyclic ring and/or optionally forms a spiro linkage with an optionally substituted, carbocyclic or heterocyclic ring;
R⁵³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{F}, —SCN, —SR^{F}, —SSR^{F}, —N₃, —NO, —N(R^{F})₂, —NO₂, —C(═O)R^{F}, —C(═O)OR^{F}, —C(═O)SR^{F}, —C(═O)N(R^{F})₂, —C(═NR^{F})R^{F}, —C(═NR^{F})OR^{F}, —C(═NR^{F})SR^{F}, —C(═NR^{F})N(R^{F})₂, —S(═O)R^{F}, —S(═O)OR^{F}, —S(═O)SR^{F}, —S(═O)N(R^{F})₂, —S(═O)₂R^{F}, —S(═O)₂OR^{F}, —S(═O)₂SR^{F}, —S(═O)₂N(R^{F})₂, —OC(═O)R^{F}, —OC(═O)OR^{F}, —OC(═O)SR^{F}, —OC(═O)N(R^{F})₂, —OC(═NR^{F})R^{F}, —OC(═NR^{F})OR^{F}, —OC(═NR^{F})SR^{F}, —OC(═NR^{F})N(R^{F})₂, —OS(═O)R^{F}, —OS(═O)OR^{F}, —OS(═O)SR^{F}, —OS(═O)N(R^{F})₂, —OS(═O)₂R^{F}, —OS(═O)₂OR^{F}, —OS(═O)₂SR^{F}, —OS(═O)₂N(R^{F})₂, —ON(R^{F})₂, —SC(═O)R^{F}, —SC(═O)OR^{F}, —SC(═O)SR^{F}, —SC(═O)N(R^{F})₂, —SC(═NR^{F})R^{F}, —SC(═NR^{F})OR^{F}, —SC(═NR^{F})SR^{F}, —SC(═NR^{F})N(R^{F})₂, —NR^{F}C(═O)R^{F}, —NR^{F}C(═O)OR^{F}, —NR^{F}C(═O)SR^{F}, —NR^{F}C(═O)N(R^{F})₂, —NR^{F}C(═NR^{F})R^{F}, —NR^{F}C(═NR^{F})OR^{F}, —NR^{F}C(═NR^{F})SR^{F}, —NR^{F}C(═NR^{F})N(R^{F})₂, —NR^{F}S(═O)R^{F}, —NR^{F}S(═O)OR^{F}, —NR^{F}S(═O)SR^{F}, —NR^{F}S(═O)N(R^{F})₂, —NR^{F}S(═O)₂R^{F}, —NR^{F}S(═O)₂OR^{F}, —NR^{F}S(═O)₂SR^{F}, —NR^{F}S(═O)₂N(R^{F})₂, —Si(R^{F})₃, —Si(R^{F})₂OR^{F}, —Si(R^{F})(OR^{F})₂, —Si(OR^{F})₃, —OSi(R^{F})₃, —Osi(R^{F})₂OR^{F}, —Osi(R^{F})(OR^{F})₂, or —Osi(OR^{F})₃;
each of R^{54a}, R^{54b}, R^{56a}, and R^{56b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl;
each instance of R^{F} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R¹ attached to the same intervening atom are joined together with the intervening atom to form an optionally substituted, monocyclic, heterocyclic or heteroaryl ring; and
R^{55a} and R^{55b} are independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{F}, —SCN, —SR^{F}, —SSR^{F}, —N₃, —NO, —N(R^{F})₂, —NO₂, —C(═O)R^{F}, —C(═O)OR^{F}, —C(═O)SR^{F}, —C(═O)N(R^{F})₂, —C(═NR^{F})R^{F}, —C(═NR^{F})OR^{F}, —C(═NR^{F})SR^{F}, —C(═NR^{F})N(R^{F})₂, —S(═O)R^{F}, —S(═O)OR^{F}, —S(═O)SR^{F}, —S(═O)N(R^{F})₂, —S(═O)₂R^{F}, —S(═O)₂OR^{F}, —S(═O)₂SR^{F}, —S(═O)₂N(R^{F})₂, —OC(═O)R^{F}, —OC(═O)OR^{F}, —OC(═O)SR^{E}, —OC(═O)N(R^{F})₂, —OC(═NR^{F})R^{F}, —OC(═NR^{F})OR^{F}, —OC(═NR^{F})SR^{F}, —OC(═NR^{F})N(R^{F})₂, —OS(═O)R^{F}, —OS(═O)OR^{F}, —OS(═O)SR^{F}, —OS(═O)N(R^{F})₂, —OS(═O)₂R^{F}, —OS(═O)₂OR^{F}, —OS(═O)₂SR^{F}, —OS(═O)₂N(R^{F})₂, —ON(R^{F})₂, —SC(═O)R^{F}, —SC(═O)OR^{F}, —SC(═O)SR^{F}, —SC(═O)N(R^{F})₂, —SC(═NR^{F})R^{F}, —SC(═NR^{F})OR^{F}, —SC(═NR^{F})SR^{F}, —SC(═NR^{F})N(R^{F})₂, —NR^{F}C(═O)R^{F}, —NR^{F}C(═O)OR^{F}, —NR^{F}C(═O)SR^{F}, —NR^{F}C(═O)N(R^{F})₂, —NR^{F}C(═NR^{F})R^{F}, —NR^{F}C(═NR^{F})OR^{F}, —NR^{F}C(═NR^{F})SR^{F}, —NR^{F}C(═NR^{F})N(R^{F})₂, —NR^{F}S(═O)R^{F}, —NR^{F}S(═O)OR^{F}, —NR^{F}S(═O)SR^{F}, —NR^{F}S(═O)N(R^{F})₂, —NR^{F}S(═O)₂R^{F}, —NR^{F}S(═O)₂OR^{F}, —NR^{F}S(═O)₂SR^{F}, —NR^{F}S(═O)₂N(R^{F})₂, —Si(R^{F})₃, —Si(R^{F})₂OR^{F}, —Si(R^{F})(OR^{F})₂, —Si(OR^{F})₃, —Osi(R^{F})₂, —Osi(R^{F})₂OR^{F}, —Osi(R^{F})(OR^{F})₂, or —Osi(OR^{F})₃;
or R^{54a} and R^{54b} are taken together with their intervening atom to form an optionally substituted, monocyclic, carbocyclic or heterocyclic ring;
or R^{54b} and R^{55a} are taken together with their intervening atom to form an optionally substituted, monocyclic, carbocyclic or heterocyclic ring;
or R^{55a} and R^{55b} are taken together with their intervening atom to form an optionally substituted, monocyclic, 5- to 7-membered heterocyclic ring;
or R^{55b} and R^{56a} are taken together with their intervening atom to form an optionally substituted, monocyclic, carbocyclic or heterocyclic ring;
or R^{56a} and R^{56b} are taken together with their intervening atom to form an optionally substituted, monocyclic, carbocyclic or heterocyclic ring;
provided that at least one of R^{55a} and R^{55b} is not —CH₃.

133. The compound of claim 132, or a pharmaceutically acceptable salt, solvate, hydrate, polymorph, co-crystal, tautomer, stereoisomer, isotopically labeled compound, or prodrug thereof, wherein each of R^{56a} and R⁵⁶⁵ is independently hydrogen, halogen, or optionally substituted alkyl.

134. The compound of claim 133, or a pharmaceutically acceptable salt, solvate, hydrate, polymorph, co-crystal, tautomer, stereoisomer, isotopically labeled compound, or prodrug thereof, wherein one of R^{56a} and R^{56b} is hydrogen.

135. The compound of any one of claims 132-134, or a pharmaceutically acceptable salt, solvate, hydrate, polymorph, co-crystal, tautomer, stereoisomer, isotopically labeled compound, or prodrug thereof, wherein R^{55a} and R^{55b} are independently hydrogen, halogen, optionally substituted alkyl, optionally substituted heteroalkyl, or optionally substituted aryl.

136. The compound of claim 135, or a pharmaceutically acceptable salt, solvate, hydrate, polymorph, co-crystal, tautomer, stereoisomer, isotopically labeled compound, or prodrug thereof, wherein one of R^{55a} and R^{55b} is hydrogen.

137. The compound of claim 135 or 136, or a pharmaceutically acceptable salt, solvate, hydrate, polymorph, co-crystal, tautomer, stereoisomer, isotopically labeled compound, or prodrug thereof, wherein R^{55a} and R^{55b} are both hydrogen.

138. The compound of any one of claims 132-134, or a pharmaceutically acceptable salt, solvate, hydrate, polymorph, co-crystal, tautomer, stereoisomer, isotopically labeled compound, or prodrug thereof, wherein R^{55a} and R^{55b} are taken together with their intervening atom to form an optionally substituted, monocyclic, 5- to 7-membered heterocyclic ring.

139. The compound of any one of claims 132-138, or a pharmaceutically acceptable salt, solvate, hydrate, polymorph, co-crystal, tautomer, stereoisomer, isotopically labeled compound, or prodrug thereof, wherein each of R^{54a} and R^{54b} is independently hydrogen, halogen, or optionally substituted alkyl.

140. The compound of claim 139, or a pharmaceutically acceptable salt, solvate, hydrate, polymorph, co-crystal, tautomer, stereoisomer, isotopically labeled compound, or prodrug thereof, wherein one of R^{54a} and R^{54b} is hydrogen.

141. The compound of any one of claims 132-140, or a pharmaceutically acceptable salt, solvate, hydrate, polymorph, co-crystal, tautomer, stereoisomer, isotopically labeled compound, or prodrug thereof, wherein R⁵¹ is optionally substituted aryl.

142. The compound of claim 141, or a pharmaceutically acceptable salt, solvate, hydrate, polymorph, co-crystal, tautomer, stereoisomer, isotopically labeled compound, or prodrug thereof, wherein R⁵¹ is 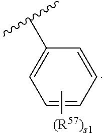
wherein:
each instance of R⁵⁷ is independently halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{D}, —SCN, —SR^{D}, —SSR^{D}, —N₃, —NO, —N(R^{D})₂, —NO₂, —C(═O)R^{D}, —C(═O)OR^{D}, —C(═O)SR^{D}, —C(═O)N(R^{D})₂, —C(═NR^{D})R^{D}, —C(═NR^{D})OR^{D}, —C(═NR^{D})SR^{D}, —C(═NR^{D})N(R^{D})₂, —S(═O)R^{D}, —S(═O)OR^{D}, —S(═O)SR^{D}, —S(═O)N(R^{D})₂, —S(═O)₂R^{D}, —S(═O)₂OR^{D}, —S(═O)₂SR^{D}, —S(═O)₂N(R^{D})₂, —OC(═O)R^{D}, —OC(═O)OR^{D}, —OC(═O)SR^{D}, —OC(═O)N(R^{D})₂, —OC(═NR^{D})R^{D}, —OC(═NR^{D})OR^{D}, —OC(═NR^{D})SR^{D}, —OC(═NR^{D})N(R^{D})₂, —OS(═O)R^{D}, —OS(═O)OR^{D}, —OS(═O)SR^{D}, —OS(═O)N(R^{D})₂, —OS(═O)₂R^{D}, —OS(═O)₂OR^{D}, —OS(═O)₂SR^{D}, —OS(═O)₂N(R^{D})₂, —ON(R^{D})₂, —SC(═O)R^{D}, —SC(═O)OR^{D}, —SC(═O)SR^{D}, —SC(═O)N(R^{D})₂, —SC(═NR^{D})R^{D}, —SC(═NR^{D})OR^{D}, —SC(═NR^{D})SR^{D}, —SC(═NR^{D})N(R^{D})₂, —NR^{D}C(═O)R^{D}, —NR^{D}C(═O)OR^{D}, —NR^{D}C(═O)SR^{D}, —NR^{D}C(═O)N(R^{D})₂, —NR^{D}C(═NR^{D})R^{D}, —NR^{D}C(═NR^{D})OR^{D}, —NR^{D}C(═NR^{D})SR^{D}, —NR^{D}C(═NR^{D})N(R^{D})₂, —NR^{D}S(═O)R^{D}, —NR^{D}S(═O)OR^{D}, —NR^{D}S(═O)SR^{D}, —NR^{D}S(═O)N(R^{D})₂, —NR^{D}S(═O)₂R^{D}, —NR^{D}S(═O)₂OR^{D}, —NR^{D}S(═O)₂SR^{D}, —NR^{D}S(═O)₂N(R^{D})₂, —Si(R^{D})₃, —Si(R^{D})₂OR^{D}, —Si(R^{D})(OR^{D})₂, —Si(OR^{D})₃, —Osi(R^{D})₃, —Osi(R^{D})₂OR^{D}, —Osi(R^{D})(OR^{D})₂, or —Osi(OR^{D})₃;
s1 is 0, 1, 2, 3, 4, or 5.

143. The compound of claim 142, or a pharmaceutically acceptable salt, solvate, hydrate, thereof, wherein R⁵¹ is

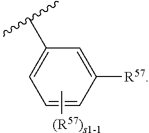

144. The compound of claim 142 or 143, or a pharmaceutically acceptable salt, solvate, hydrate, polymorph, co-crystal, tautomer, stereoisomer, isotopically labeled compound, or prodrug thereof, wherein s1 is 1.

145. The compound of any one of claims 132-144, or a pharmaceutically acceptable salt, solvate, hydrate, polymorph, co-crystal, tautomer, stereoisomer, isotopically labeled compound, or prodrug thereof, wherein R⁵² is optionally substituted alkyl.

146. The compound of claim 145, or a pharmaceutically acceptable salt, solvate, hydrate, polymorph, co-crystal, tautomer, stereoisomer, isotopically labeled compound, or prodrug thereof, wherein R⁵² is unsubstituted C₁-C₄ alkyl.

147. The compound of any one of claims 132-140, or a pharmaceutically acceptable salt, solvate, hydrate, polymorph, co-crystal, tautomer, stereoisomer, isotopically labeled compound, or prodrug thereof, wherein R⁵¹ and R⁵² are taken together with their intervening atom to form an optionally substituted, monocyclic, carbocyclic ring.

148. The compound of any one of claims 132-140, or a pharmaceutically acceptable salt, solvate, hydrate, polymorph, co-crystal, tautomer, stereoisomer, isotopically labeled compound, or prodrug thereof, wherein R⁵¹ and R⁵² are taken together with their intervening atom to form an optionally substituted, monocyclic, heterocyclic ring.

149. The compound of claim 148, or a pharmaceutically acceptable salt, solvate, hydrate, polymorph, co-crystal, tautomer, stereoisomer, isotopically labeled compound, or prodrug thereof, wherein R⁵¹ and R⁵² are taken together with their intervening atom to form an optionally substituted tetrahydropyranyl.

150. The compound of any one of claims 132-149, or a pharmaceutically acceptable salt, solvate, hydrate, polymorph, co-crystal, tautomer, stereoisomer, isotopically labeled compound, or prodrug thereof, wherein R⁵³ is hydrogen, optionally substituted C₁-C₆ alkyl, or halogen.

151. The compound of claim 150, or a pharmaceutically acceptable salt, solvate, hydrate, polymorph, co-crystal, tautomer, stereoisomer, isotopically labeled compound, or prodrug thereof, wherein R⁵³ is hydrogen, fluorine, —CH₃, —CH₂F, —CHF₂, or —CF₃.

152. The compound of claim 132, or a pharmaceutically acceptable salt, solvate, hydrate, polymorph, co-crystal, tautomer, stereoisomer, isotopically labeled compound, or prodrug thereof, wherein the compound is any one of the formulae shown in Table 11:
TABLE 11

| | |
|---|---|
| 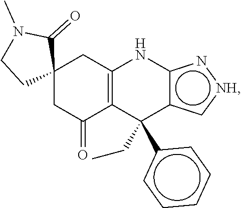 | 949753 |
| 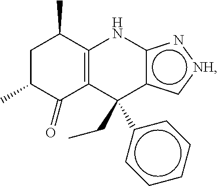 | 865199 |
| 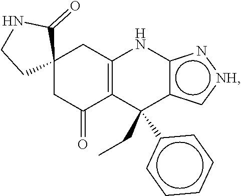 | 950161 |
| 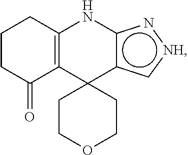 | 884111 |
| 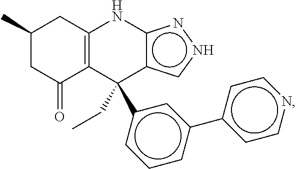 | 874315 |
| 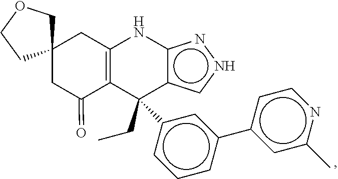 | 947412 |
| 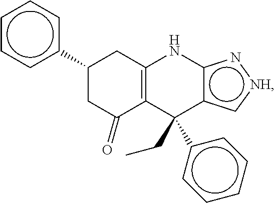 | 791265 |
| 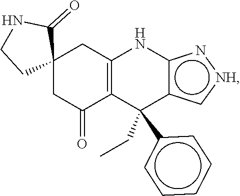 | 950160 |
| 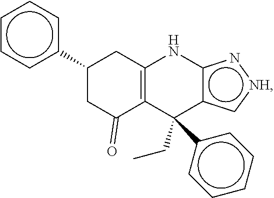 | 791264 |
| 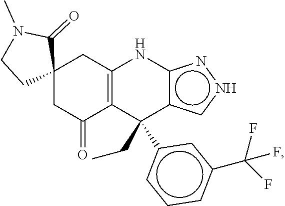 | 950330 |
| 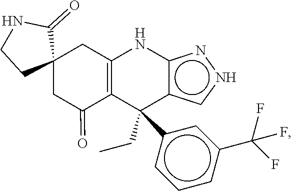 | 950331 |
| 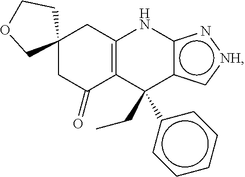 | 816884 |
| 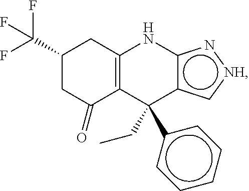 | 806608 |
| 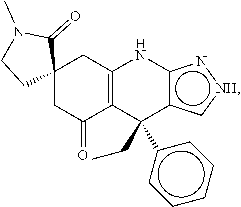 | 949760 |
| 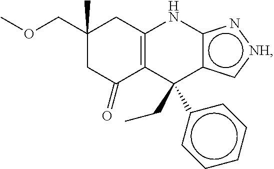 | 816875 |
| 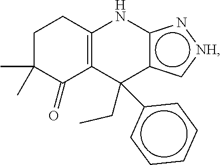 | 835310 |
| 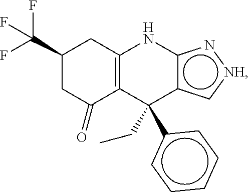 | 806609 |
| 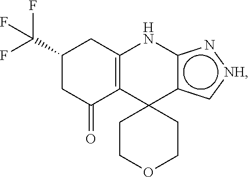 | 901477 |
| 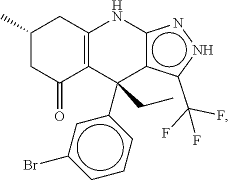 | 828849 |
| 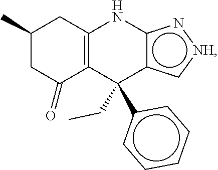 | 789013 |
| 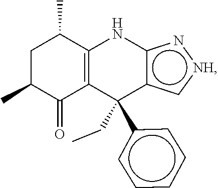 | 865198 |
| 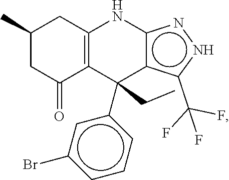 | 828850 |
| 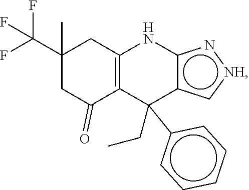 | 816796 |
| 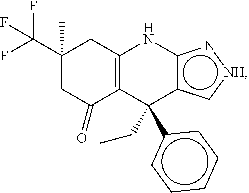 | 819569 |
| 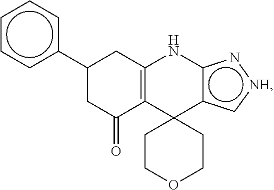 | 893309 |
| 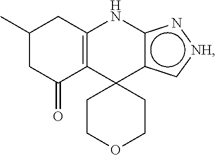 | 884112 |
| 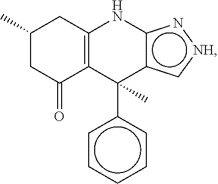 | 789334 |
| 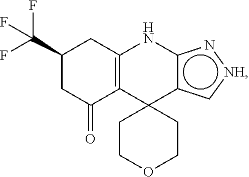 | 901478 |
| 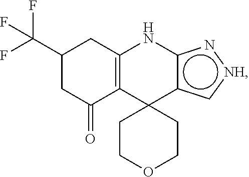 | 889572 |
| 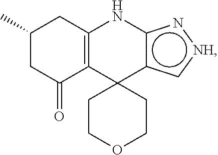 | 912078 |
| 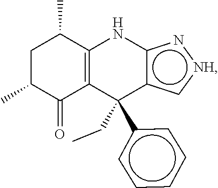 | 909217 |
| 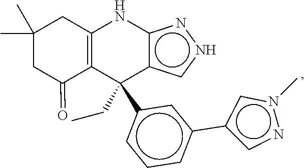 | 823724 |
| 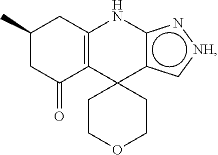 | 912079 |
| 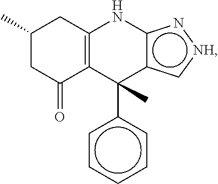 | 789332 |
| 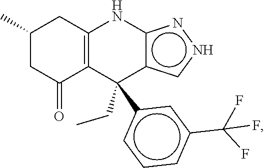 | 810452 |
| 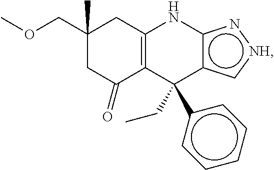 | 816873 |
| 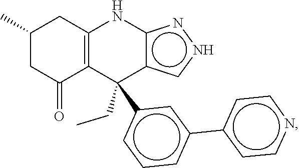 | 874316 |
| 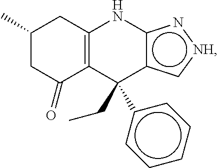 | 789010 |
| 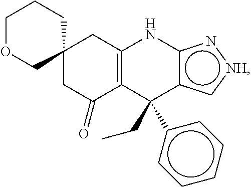 | 816878 |
| 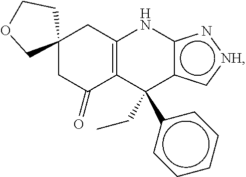 | 816881 |
| 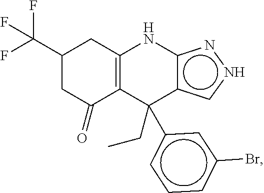 | 819565 |
| 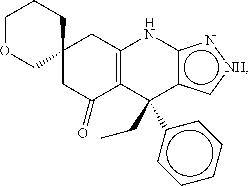 | 816879 |
| 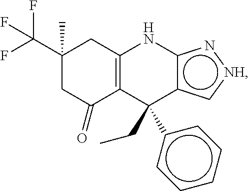 | 819568 |
| 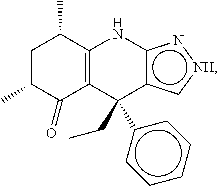 | 909216 |
| 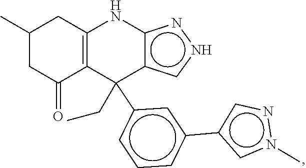 | 816752 |
| 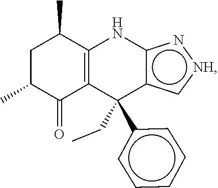 | 865201 |
| 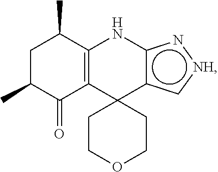 | 893316 |
| 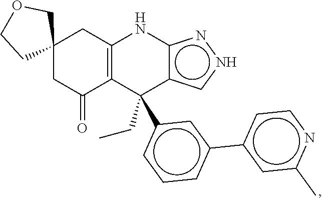 | 947407 |
| 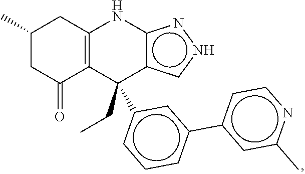 | 932657 |
| 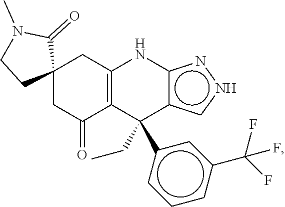 | 950326 |
| 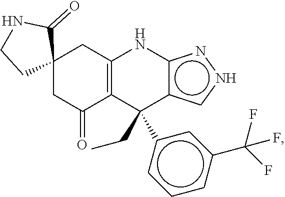 | 950333 |
| 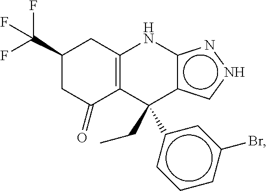 | 826653 |
| 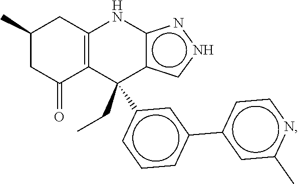 | 932658 |
| 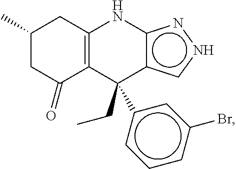 | 810486 |
| 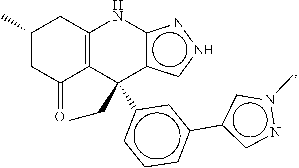 | 823720 |
| 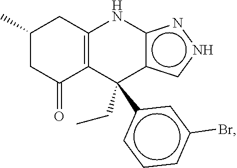 | 810487 |
| 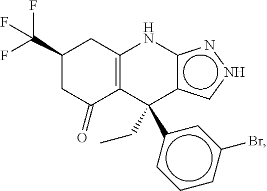 | 826652 |
| 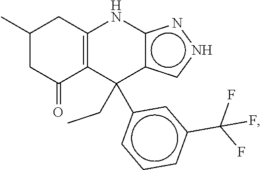 | 801397 |
| 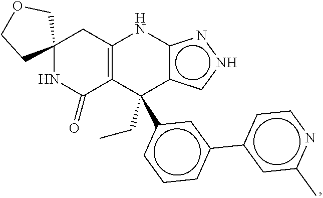 | 947406 |
| 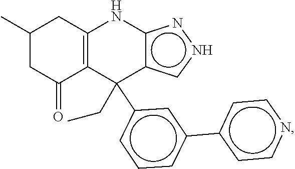 | 856947 |
| 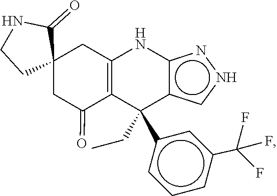 | 950324 |
| 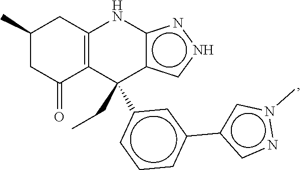 | 823721 |
| 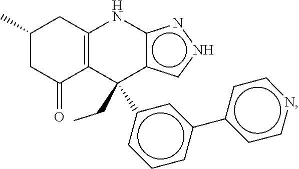 | 874318 |
| 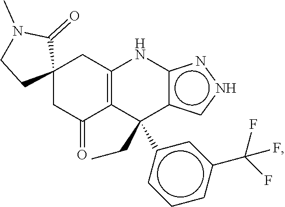 | 950329 |
| 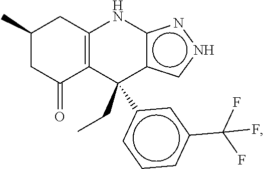 | 810451 |
| 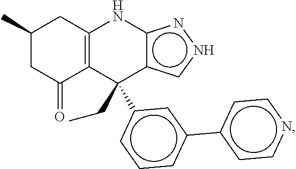 | 874317 |
| 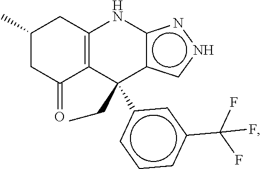 | 810450 |
| 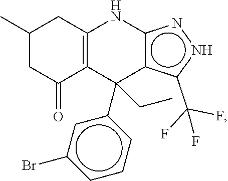 | 826715 |
| 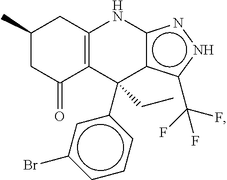 | 828848 |
| 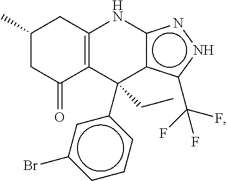 | 828847 |
| 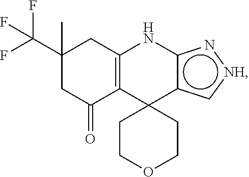 | 901458 |
| 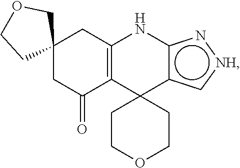 | 901476 |
| 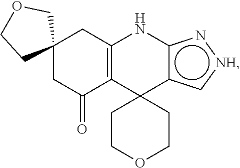 | 901475 |
| 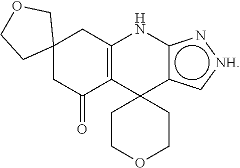 | 889573 |

153. The compound of claim 132, or a pharmaceutically acceptable salt, solvate, hydrate, polymorph, co-crystal, tautomer, stereoisomer, isotopically labeled compound, or prodrug thereof, wherein the compound is any one of the formulae shown in Table 12:
TABLE 12 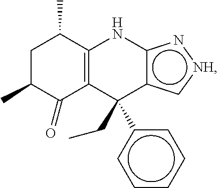
865202 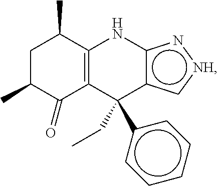
909218 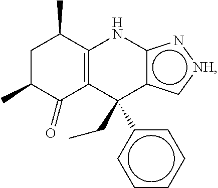
909219 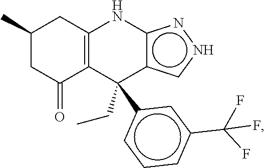
810453 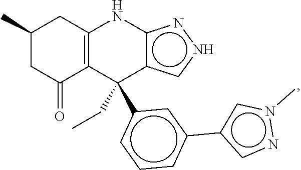
823726 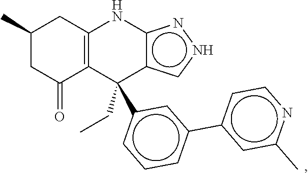
932637 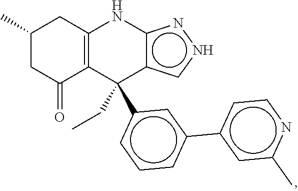
932638 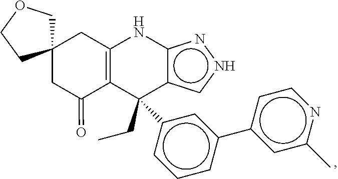
947411 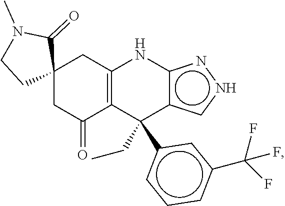
950325 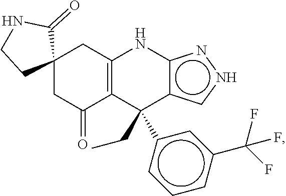
950332 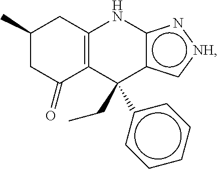
789009 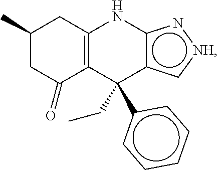
789011 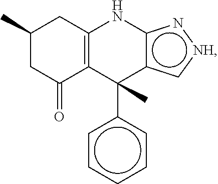
789331 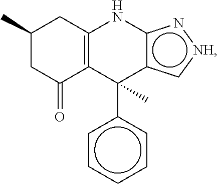
789333 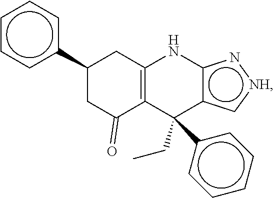
791262 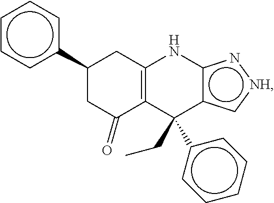
791263 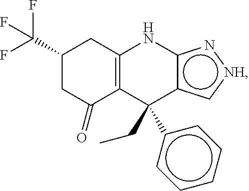
806607 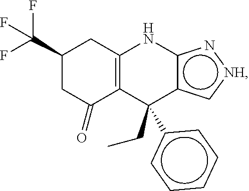
806611 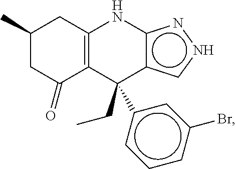
810484 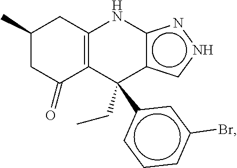
810485 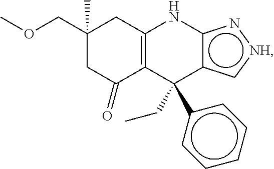
816872 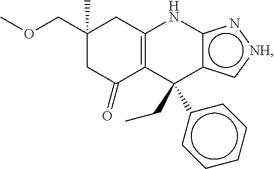
816874 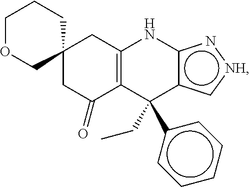
816876 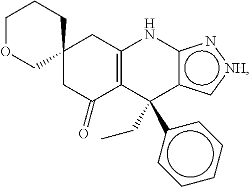
816877 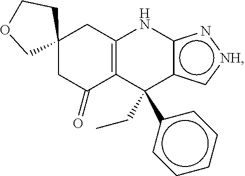
816880 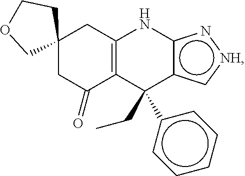
816882 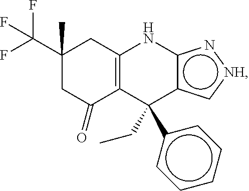
819570 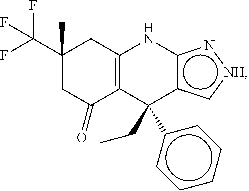
819571 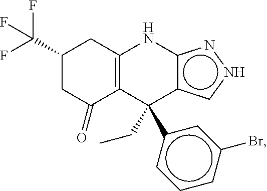
826654 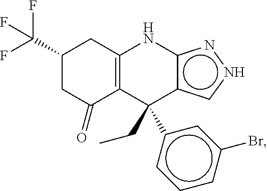
826655 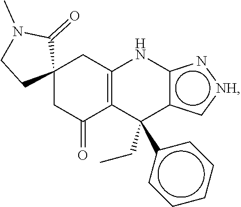
949754 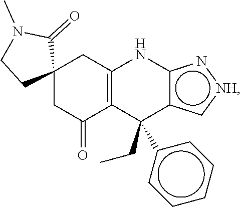
949759 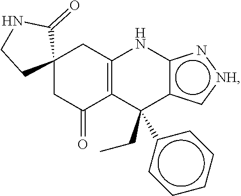
950164 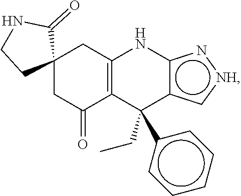
950165.

154. The compound of claim 132, or a pharmaceutically acceptable salt, solvate, hydrate, polymorph, co-crystal, tautomer, stereoisomer, isotopically labeled compound, or prodrug thereof, wherein the compound is not a compound of claim 153.

155. A compound of any one of the formulae shown in Table 13:
TABLE 13

| | |
|---|---|
| 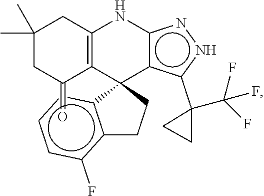 | 816839 |
| 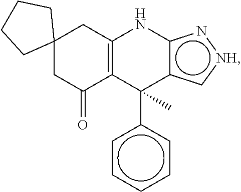 | 792339 |
| 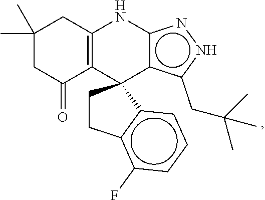 | 791256 |
| 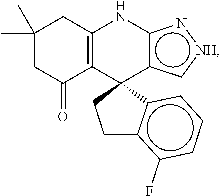 | 806632 |
| 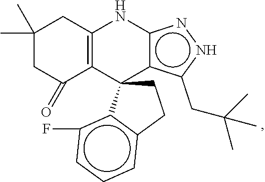 | 801351 |
| 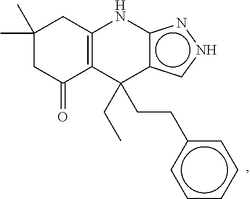 | 819563 |
| 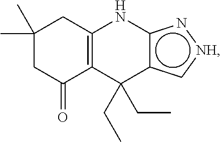 | 810505 |
| 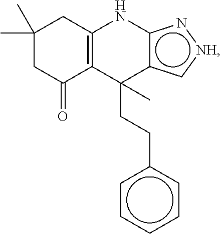 | 819562 |
| 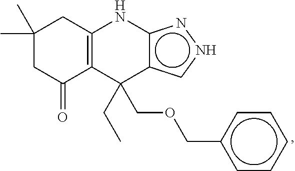 | 874313 |
| 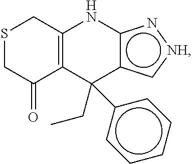 | 801406 |
| 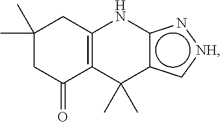 | 786065 |
| 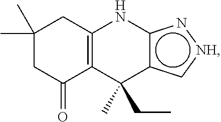 | 884019 |
| 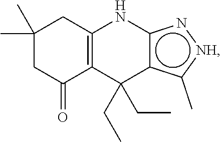 | 786061 |
| 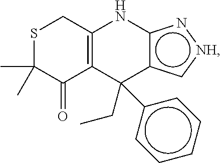 | 862028 |
| 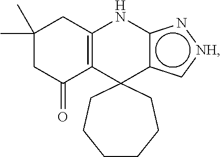 | 883978 |
| 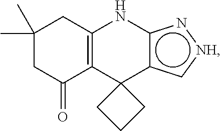 | 874509 |
| 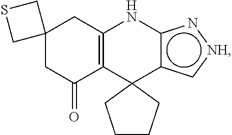 | 913441 |
| 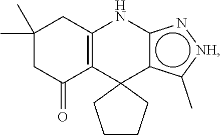 | 786072 |
| 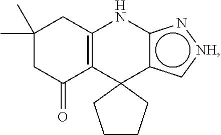 | 790666 |
| 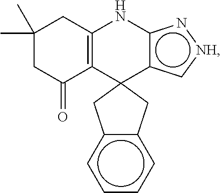 | 897752 |
| 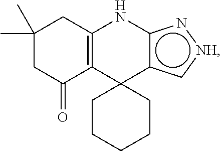 | 826650 |
| 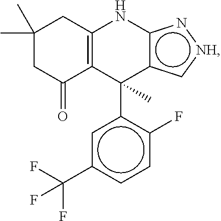 | 816797 |
| 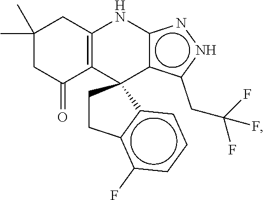 | 816837 |
| 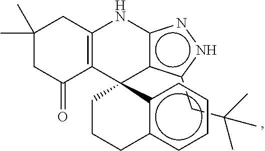 | 811596 |
| 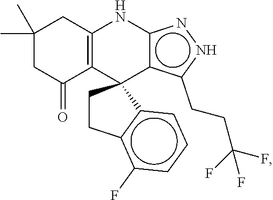 | 874323 |
| 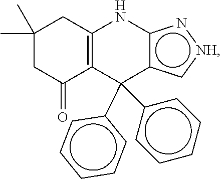 | 789461 |
| 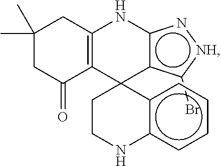 | 810460 |
| 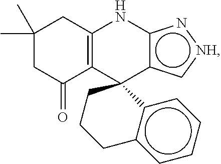 | 930565 |
| 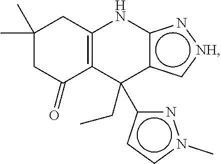 | 790619 |
| 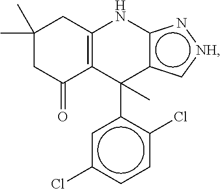 | 806605 |
| 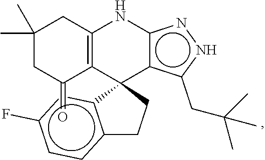 | 789504 |
| 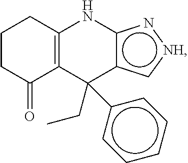 | 789497 |
| 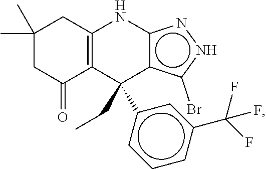 | 810464 |
| 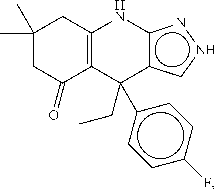 | 772965 |
| 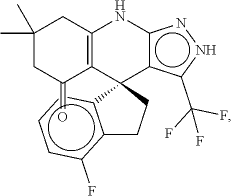 | 806568 |
| 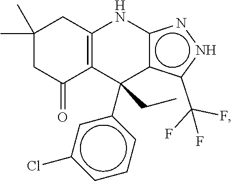 | 801309 |
| 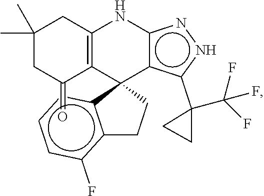 | 806840 |
| 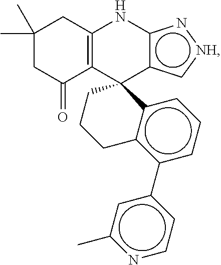 | 945550 |
| 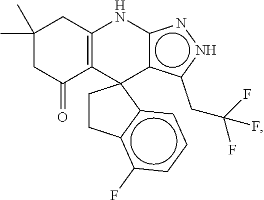 | 810590 |
| 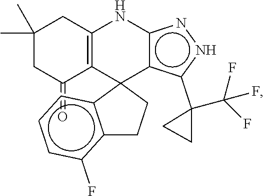 | 810591 |
| 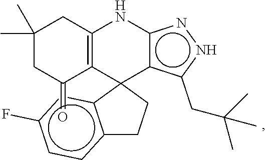 | 784440 |
| 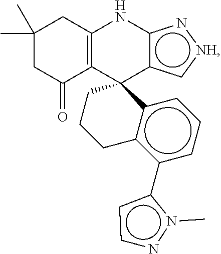 | 945552 |
| 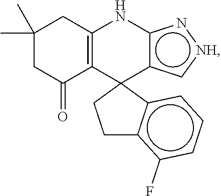 | 801357 |
| 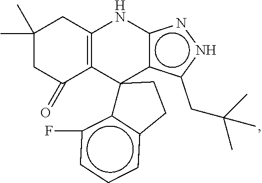 | 789583 |
| 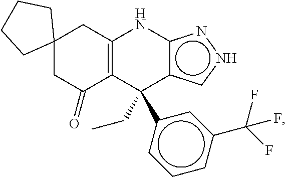 | 801310 |
| 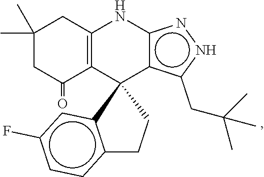 | 789505 |
| 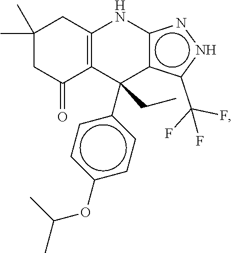 | 947873 |
| 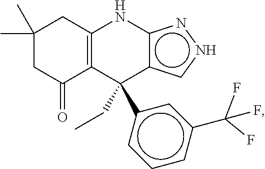 | 790755 |
| 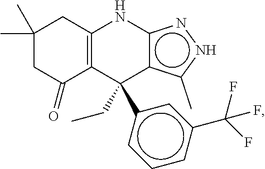 | 810368 |
| 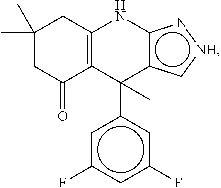 | 785735 |
| 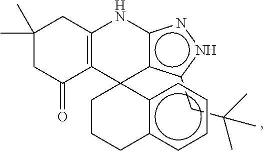 | 810495 |
| 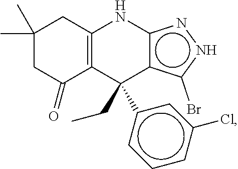 | 801315 |
| 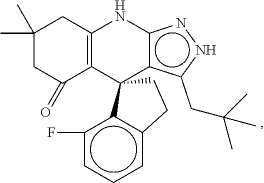 | 801352 |
| 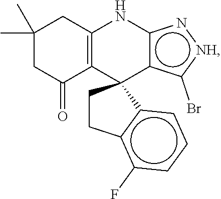 | 806938 |
| 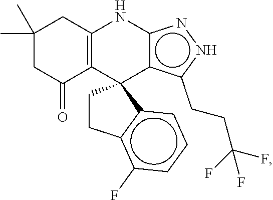 | 874324 |
| 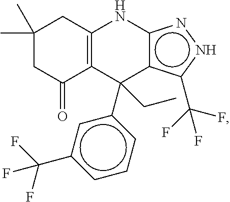 | 789584 |
| 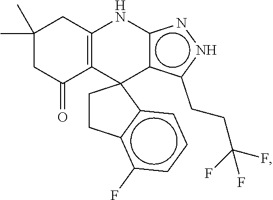 | 841868 |
| 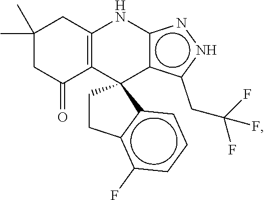 | 816838 |
| 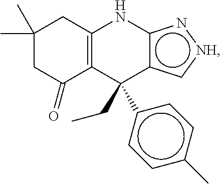 | 789457 |
| 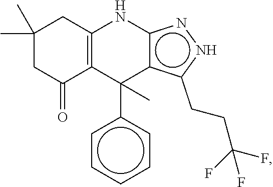 | 828845 |
| 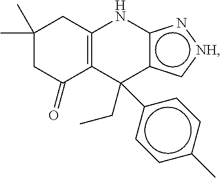 | 785739 |
| 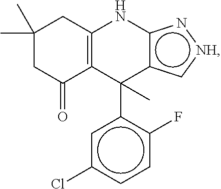 | 810377 |
| 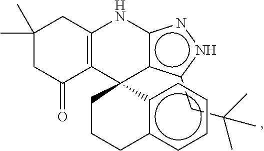 | 811595 |
| 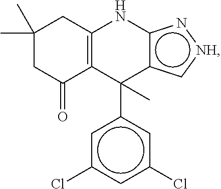 | 789337 |
| 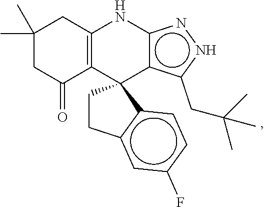 | 782425 |
| 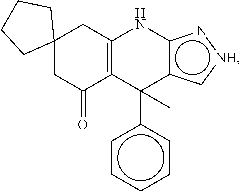 | 789460 |
| 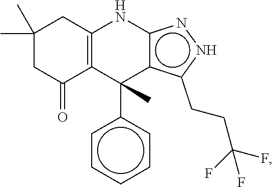 | 856949 |
| 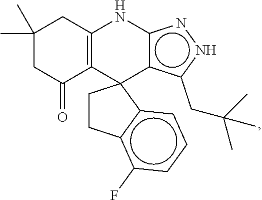 | 789524 |
| 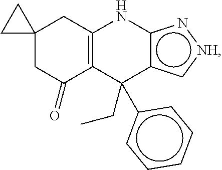 | 789330 |
| 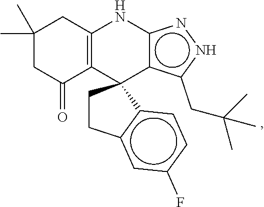 | 782424 |
| 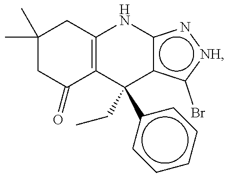 | 792341 |
| 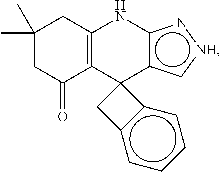 | 917226 |
| 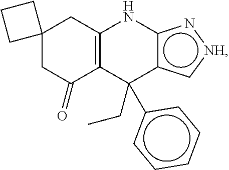 | 811555 |
| 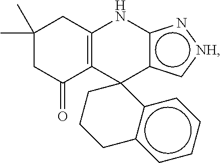 | 913219 |
| 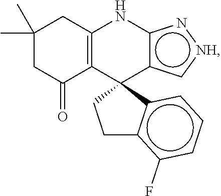 | 806633 |
| 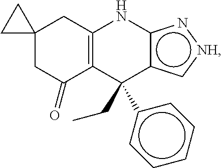 | 790615 |
| 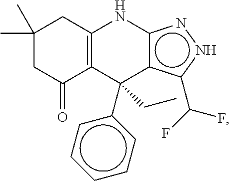 | 874511 |
| 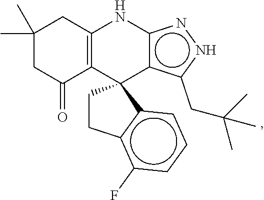 | 791257 |
| 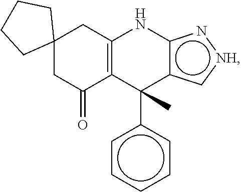 | 792338 |
| 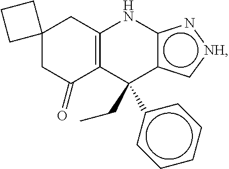 | 819573 |
| 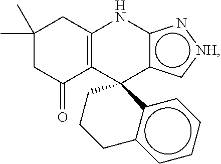 | 930566 |
| 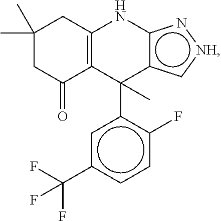 | 810376 |
| 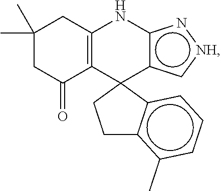 | 806603 |
| 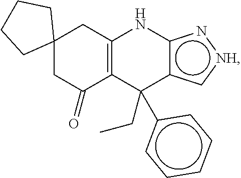 | 785737 |
| 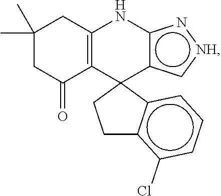 | 816795 |
| 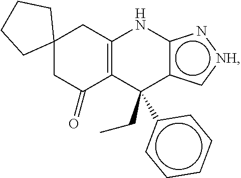 | 789562 |
| 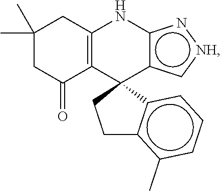 | 913136 |
| 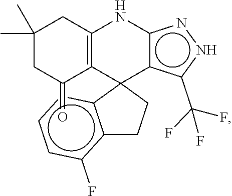 | 801356 |
| 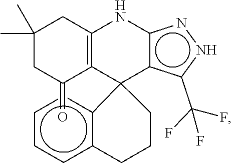 | 790745 |
| 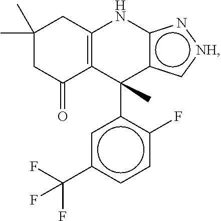 | 816798 |
| 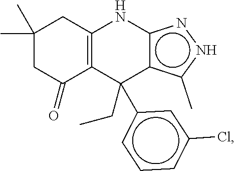 | 789336 |
| 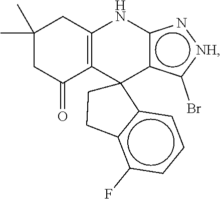 | 801358 |
| 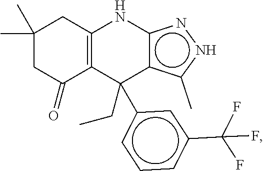 | 801321 |
| 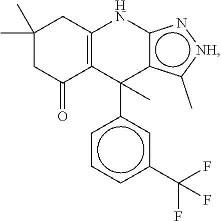 | 811556 |
| 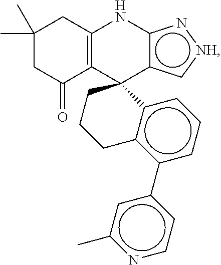 | 945549 |
| 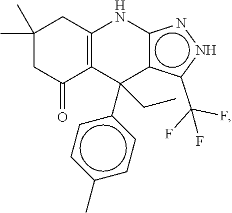 | 883866 |
| 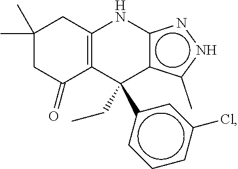 | 790612 |
| 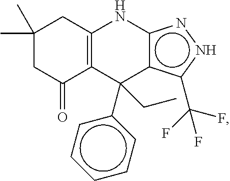 | 792343 |
| 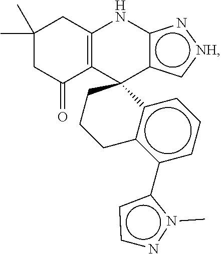 | 945551 |
| 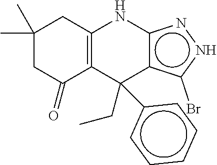 | 789459 |
| 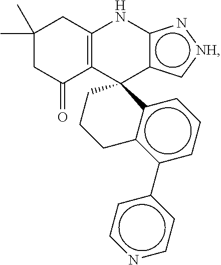 | 945548 |
| 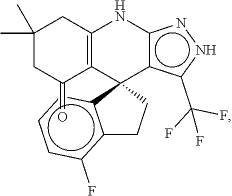 | 806569 |
| 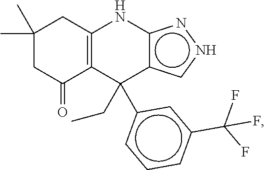 | 785762 |
| 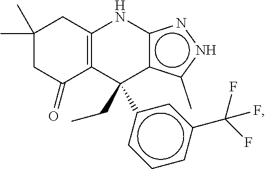 | 810367 |
| 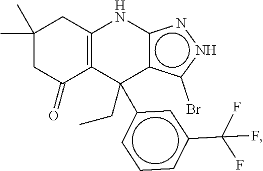 | 802358 |
| 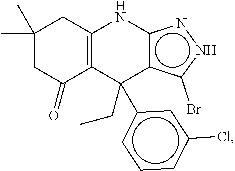 | 789564 |
| 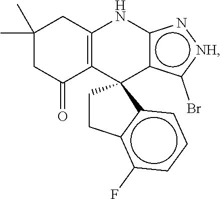 | 806939 |
| 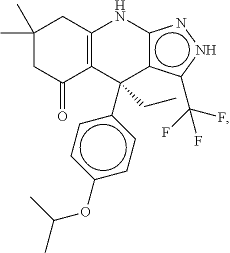 | 947868 |
| 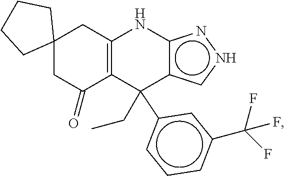 | 790617 |
| 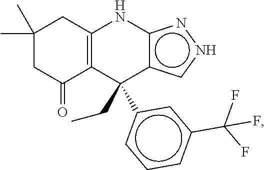 | 790754 |
| 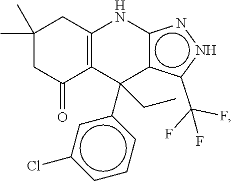 | 789566 |
| 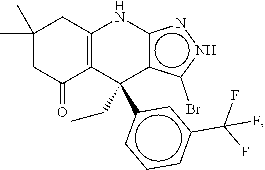 | 810466 |
| 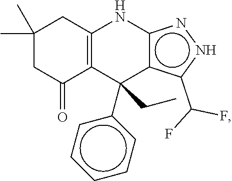 | 874510 |
| 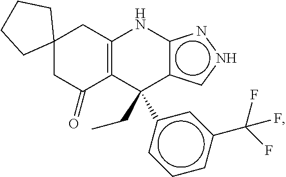 | 801311 |
| 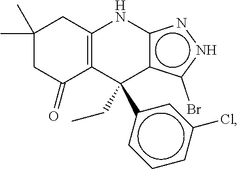 | 801314 |
| 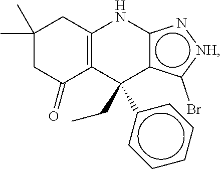 | 792340 |
| 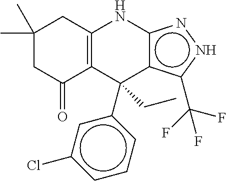 | 801308 |
| 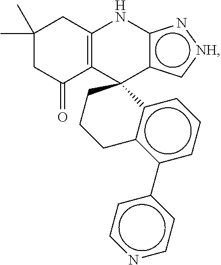 | 945547 |

or a pharmaceutically acceptable salt, solvate, hydrate, polymorph, co-crystal, tautomer, stereoisomer, isotopically labeled compound, or prodrug thereof.

156. A compound of any one of the formulae shown in Table 13A:
TABLE 13A 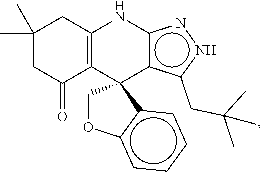
117093 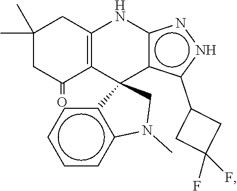
119047 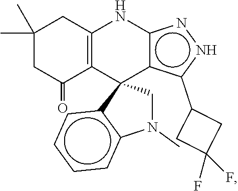
119048 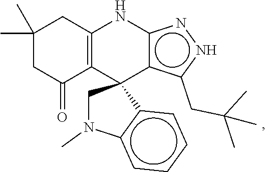
125978 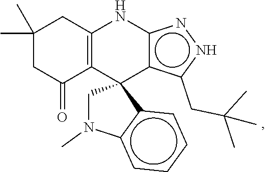
125979 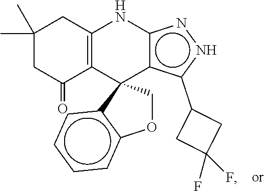
127429 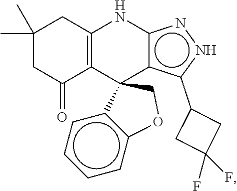
127430
or a pharmaceutically acceptable salt, solvate, hydrate, polymorph, co-crystal, tautomer, stereoisomer, isotopically labeled compound, or prodrug thereof.

157. A compound of any one of the formulae shown in Table 14:
TABLE 14 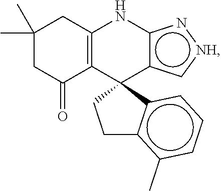
913137 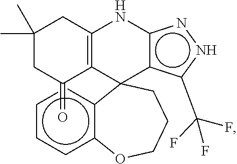
790632 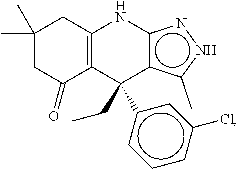
790611 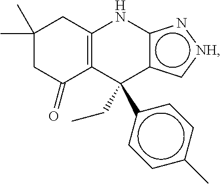
789458 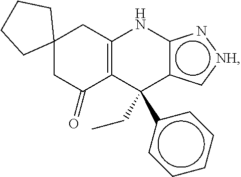
789563 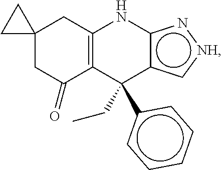
790616 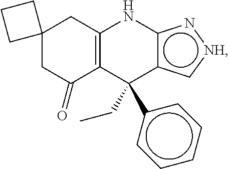
819572 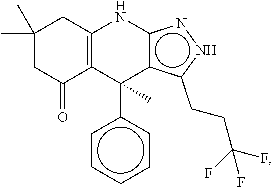
856948 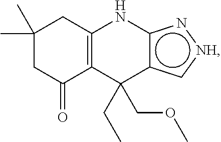
841866 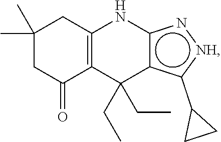
877294 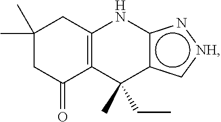
884020 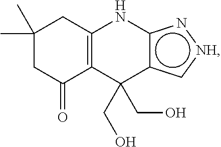
889570 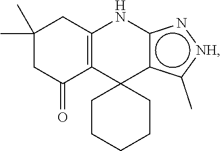
877288 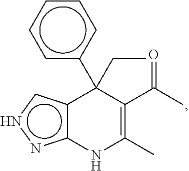
806940 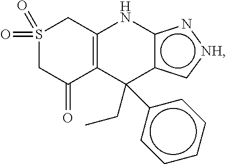
811512 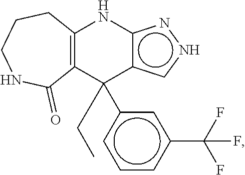
913355
or a pharmaceutically acceptable salt, solvate, hydrate, polymorph, co-crystal, tautomer, stereoisomer, isotopically labeled compound, or prodrug thereof.

158. A compound of the formula: 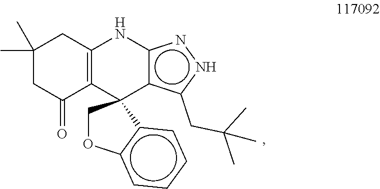
or a pharmaceutically acceptable salt, solvate, hydrate, polymorph, co-crystal, tautomer, stereoisomer, isotopically labeled compound, or prodrug thereof.

159. The compound of any one of claims 1-158, or a pharmaceutically acceptable salt thereof.

160. A pharmaceutical composition comprising:
the compound of any one of claims 1-159, or a pharmaceutically acceptable salt, solvate, hydrate, polymorph, co-crystal, tautomer, stereoisomer, isotopically labeled compound, or prodrug thereof, and
optionally a pharmaceutically acceptable excipient.

161. The pharmaceutical composition of claim 160 further comprising an additional pharmaceutical agent.

162. A kit comprising:
the compound of any one of claims 1-159, or a pharmaceutically acceptable salt, solvate, hydrate, polymorph, co-crystal, tautomer, stereoisomer, isotopically labeled compound, or prodrug thereof, or the pharmaceutical composition of claim 160 or 161; and
instructions for using the compound, or pharmaceutically acceptable salt, solvate, hydrate, polymorph, co-crystal, tautomer, stereoisomer, isotopically labeled compound, or prodrug thereof, or pharmaceutical composition.

163. A method of treating a disease in a subject in need thereof, the method comprising administering to the subject an effective amount of:
the compound of any one of claims 1-159, or pharmaceutically acceptable salt, solvate, hydrate, polymorph, co-crystal, tautomer, stereoisomer, isotopically labeled compound, or prodrug thereof;
a compound of the formula: 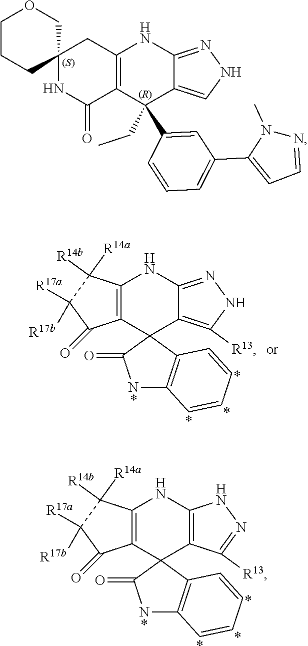
or a pharmaceutically acceptable salt, solvate, hydrate, polymorph, co-crystal, tautomer,
each instance of the atoms marked with * is independently optionally substituted; [symbol]
is a single or double bond;
R¹³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{B}, —SCN, —SR^{B}, —SSR^{B}, —N₃, —NO, —N(R^{B})₂, —NO₂, —C(═O)R^{B}, —C(═O)OR^{B}, —C(═O)SR^{B}, —C(═O)N(R^{B})₂, —C(═NR^{B})R^{B}, —C(═NR^{B})OR^{B}, —C(═NR^{B})SR^{B}, —C(═NR^{B})N(R^{B})₂, —S(═O)R^{B}, —S(═O)OR^{B}, —S(═O)SR^{B}, —S(═O)N(R^{B})₂, —S(═O)₂R^{B}, —S(═O)₂OR^{B}, —S(═O)₂SR^{B}, —S(═O)₂N(R^{B})₂, —OC(═O)R^{B}, —OC(═O)OR^{B}, —OC(═O)SR^{B}, —OC(═O)N(R^{B})₂, —OC(═NR^{B})R^{B}—OC(═NR^{B})OR^{B}, —OC(═NR^{B})SR^{B}, —OC(═NR^{B})N(R^{B})₂, —OS(═O)R^{B}, —OS(═O)OR^{B}, —OS(═O)SR^{B}, —OS(═O)N(R^{B})₂, —OS(═O)₂R^{B}, —OS(═O)₂OR^{B}, —OS(═O)₂SR^{B}, —OS(═O)₂N(R^{B})₂, —ON(R^{B})₂, —SC(═O)R^{B}, —SC(═O)OR^{B}, —SC(═O)SR^{B}, —SC(═O)N(R^{B})₂, —SC(═NR^{B})R^{B}, —SC(═NR^{B})OR^{B}, —SC(═NR^{B})SR^{B}, —SC(═NR^{B})N(R^{B})₂, —NR^{B}C(═O)R^{B}, —NR^{B}C(═O)OR^{B}, —NR^{B}C(═O)SR^{B}, —NR^{B}C(═O)N(R^{B})₂, —NR^{B}C(═NR^{B})R^{B}, —NR^{B}C(═NR^{B})OR^{B}, —NR^{B}C(═NR^{B})SR^{B}, —NR^{B}C(═NR^{B})N(R^{B})₂, —NR^{B}S(═O)R^{B}, —NR^{B}S(═O)OR^{B}, —NR^{B}S(═O)SR^{B}, —NR^{B}S(═O)N(R^{B})₂, —NR^{B}S(═O)₂R^{B}, —NR^{B}S(═O)₂OR^{B}, —NR^{B}S(═O)₂SR^{B}, —NR^{B}S(═O)₂N(R^{B})₂, —Si(R^{B})₃, —Si(R^{B})₂OR^{B}, —Si(R^{B})(OR^{B})₂, —Si(OR^{B})₃, —OSi(R^{B})₃, —OSi(R^{B})₂OR^{B}, —OSi(R^{B})(OR^{B})₂, or —OSi(OR^{B})₃;
each of R^{14a}, R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl; and
each instance of R^{B} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{B} attached to the same intervening atom are joined together with the intervening atom to form an optionally substituted, monocyclic, heterocyclic or heteroaryl ring; or
the pharmaceutical composition of claim 160 or 161.

164. A method of preventing a disease in a subject in need thereof, the method comprising administering to the subject an effective amount of:
the compound of any one of claims 1-159, or pharmaceutically acceptable salt, solvate, hydrate, polymorph, co-crystal, tautomer, stereoisomer, isotopically labeled compound, or prodrug thereof;
a compound of the formula: 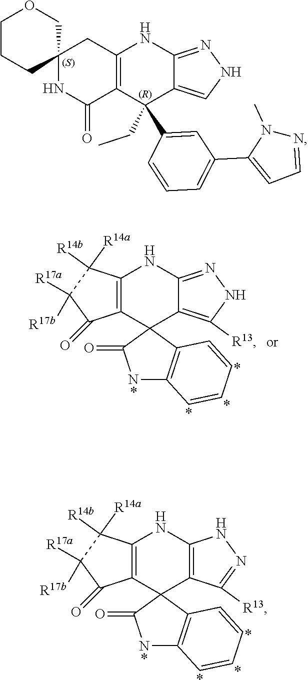
or a pharmaceutically acceptable salt, solvate, hydrate, polymorph, co-crystal, tautomer, stereoisomer, isotopically labeled compound, or prodrug thereof, wherein:
each instance of the atoms marked with * is independently optionally substituted; [symbol]
is a single or double bond;
R¹³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{B}, —SCN, —SR^{B}, —SSR^{B}, —N₃, —NO, —N(R^{B})₂, —NO₂, —C(═O)R^{B}, —C(═O)OR^{B}, —C(═O)SR^{B}, —C(═O)N(R^{B})₂, —C(═NR^{B})R^{B}, —C(═NR^{B})OR^{B}, —C(═NR^{B})SR^{B}, —C(═NR^{B})N(R^{B})₂, —S(═O)R^{B}, —S(═O)OR^{B}, —S(═O)SR^{B}, —S(═O)N(R^{B})₂, —S(═O)₂R^{B}, —S(═O)₂OR^{B}, —S(═O)₂SR^{B}, —S(═O)₂N(R^{B})₂, —OC(═O)R^{B}, —OC(═O)OR^{B}, —OC(═O)SR^{B}, —OC(═O)N(R^{B})₂, —OC(═NR^{B})R^{B}, —OC(═NR^{B})OR^{B}, —OC(═NR^{B})SR^{B}, —OC(═NR^{B})N(R^{B})₂, —OS(═O)R^{B}, —OS(═O)OR^{B}, —OS(═O)SR^{B}, —OS(═O)N(R^{B})₂, —OS(═O)₂R^{B}, —OS(═O)OR^{B}, —OS(═O)₂SR^{B}, —OS(═O)₂N(R^{B})₂, —ON(R^{B})₂, —SC(═O)R^{B}, —SC(═O)OR^{B}, —SC(═O)SR^{B}, —SC(═O)N(R^{B})₂, —SC(═NR^{B})R^{B}, —SC(═NR^{B})OR^{B}, —SC(═NR^{B})SR^{B}, —SC(═NR^{B})N(R^{B})₂, —NR^{B}C(═O)R^{B}, —NR^{B}C(═O)OR^{B}, —NR^{B}C(═O)SR^{B}, —NR^{B}C(═O)N(R^{B})₂, —NR^{B}C(═NR^{B})R^{B}, —NR^{B}C(═NR^{B})OR^{B}, —NR^{B}C(═NR^{B})SR^{B}, —NR^{B}C(═NR^{B})N(R^{B})₂, —NR^{B}S(═O)R^{B}, —NR^{B}S(═O)OR^{B}, —NR^{B}S(═O)SR^{B}, —NR^{B}S(═O)N(R^{B})₂, —NR^{B}S(═O)₂R^{B}, —NR^{B}S(═O)₂OR^{B}, —NR^{D}S(═O)₂SR^{B}, —NR^{B}S(═O)₂N(R^{B})₂, —Si(R^{B})₃, —Si(R^{B})₂OR^{B}, —Si(R^{B})(OR^{B})₂, —Si(OR^{B})₃, —OSi(R^{B})₃, —OSi(R^{B})₂OR^{B}, —OSi(R^{B})(OR^{B})₂, or —OSi(OR^{B})₃;
each of R^{14a}, R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl; and
each instance of R^{B} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{B} attached to the same intervening atom are joined together with the intervening atom to form an optionally substituted, monocyclic, heterocyclic or heteroaryl ring; or
the pharmaceutical composition of claim 160 or 161.

165. The method of claim 163 or 164, wherein the disease is associated with aberrantly high activity and/or production of a glycogen synthase kinase 3 (GSK3).

166. The method of any one of claims 163-165, wherein the effective amount is effective in inhibiting the activity and/or production of the GSK3.

167. The method of any one of claims 163-166, wherein the disease is associated with aberrantly high activity and/or production of catenin beta-1.

168. The method of any one of claims 163-167, wherein the disease is associated with a mutation and/or overexpression of the CTNNB1 gene.

169. The method of any one of claims 163-168, wherein the disease is a mental or behavioral disease.

170. The method of claim 169, wherein the disease is fragile X syndrome.

171. The method of claim 169, wherein the disease is autism.

172. The method of claim 169, wherein the disease is schizophrenia.

173. The method of claim 169, wherein the disease is bipolar disorder.

174. The method of claim 169, wherein the disease is attention deficit hyperactivity disorder.

175. The method of any one of claims 163-168, wherein the disease is a neurological disease.

176. The method of claim 175, wherein the disease is seizure.

177. The method of claim 175, wherein the disease is Alzheimer's disease.

178. The method of claim 175, wherein the disease is Huntington's disease.

179. The method of claim 175, wherein the disease is Parkinson's disease.

180. The method of claim 175, wherein the disease is amyotrophic lateral sclerosis.

181. The method of any one of claims 163-168, wherein the disease is a cancer.

182. The method of claim 181, wherein the disease is a hematological malignancy.

183. The method of claim 181, wherein the disease is leukemia.

184. The method of claim 181, wherein the disease is acute myeloid leukemia.

185. The method of claim 181, wherein the disease is acute lymphoblastic leukemia.

186. The method of claim 181, wherein the disease is colon cancer.

187. The method of claim 181, wherein the disease is pancreatic cancer.

188. The method of any one of claims 163-168, wherein the disease is a metabolic disease.

189. The method of claim 188, wherein the disease is diabetes.

190. The method of claim 188, wherein the disease is Type II diabetes.

191. The method of claim 188, wherein the disease is obesity.

192. A method of inhibiting the activity and/or production of a glycogen synthase kinase 3 (GSK3) in a subject in need thereof, the method comprising administering to the subject an effective amount of:
the compound of any one of claims 1-159, or pharmaceutically acceptable salt, solvate, hydrate, polymorph, co-crystal, tautomer, stereoisomer, isotopically labeled compound, or prodrug thereof,
a compound of the formula: 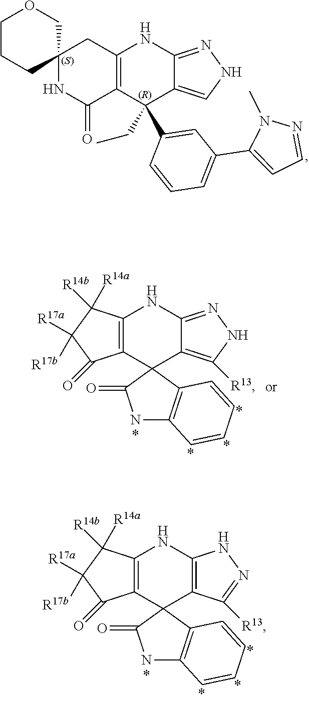
or a pharmaceutically acceptable salt, solvate, hydrate, polymorph, co-crystal, tautomer, stereoisomer, isotopically labeled compound, or prodrug thereof, wherein:
each instance of the atoms marked with * is independently optionally substituted; [symbol]
is a single or double bond;
R¹³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{B}, —SCN, —SR^{B}, —SSR^{B}, —N₃, —NO, —N(R^{B})₂, —NO₂, —C(═O)R^{B}, —C(═O)OR^{B}, —C(═O)SR^{B}, —C(═O)N(R^{B})₂, —C(═NR^{B})R^{B}, —C(═NR^{B})OR^{B}, —C(═NR^{B})SR^{B}, —C(═NR^{B})N(R^{B})₂, —S(═O)R^{B}, —S(═O)OR^{B}, —S(═O)SR^{B}, —S(═O)N(R^{B})₂, —S(═O)₂R^{B}, —S(═O)₂OR^{B}, —S(═O)₂SR^{B}, —S(═O)₂N(R^{B})₂, —OC(═O)R^{B}, —OC(═O)OR^{B}, —OC(═O)SR^{B}, —OC(═O)N(R^{B})₂, —OC(═NR^{B})R^{B}, —OC(═NR^{B})OR^{B}, —OC(═NR^{B})SR^{B}, —OC(═NR^{B})N(R^{B})₂, —OS(═O)R^{B}, —OS(═O)OR^{B}, —OS(═O)SR^{B}, —OS(═O)N(R^{B})₂, —OS(═O)₂R^{B}, —OS(═O)₂OR^{B}, —OS(═O)₂SR^{B}, —OS(═O)₂N(R^{B})₂, —ON(R^{B})₂, —SC(═O)R^{B}, —SC(═O)OR^{B}, —SC(═O)SR^{B}, —SC(═O)N(R^{B})₂, —SC(═NR^{B})R^{B}, —SC(═NR^{B})OR^{B}, —SC(═NR^{B})SR^{B}, —SC(═NR^{B})N(R^{B})₂, —NR^{B}C(═O)R^{B}, —NR^{B}C(═O)OR^{B}, —NR^{B}C(═O)SR^{B}, —NR^{B}C(═O)N(R^{B})₂, —NR^{B}C(═NR^{B})R^{B}, —NR^{B}C(═NR^{B})OR^{B}, —NR^{B}C(═NR^{B})SR^{B}, —NR^{B}C(═NR^{B})N(R³)₂, —NR^{B}S(═O)R^{B}, —NR^{B}S(═O)OR^{B}, —NR^{B}S(═O)SR^{B}, —NR^{B}S(═O)N(R^{B})₂, —NR^{B}S(═O)₂R^{B}, —NR^{B}S(═O)₂OR^{B}, —NR^{B}S(═O)₂SR^{B}, —NR^{B}S(═O)₂N(R^{B})₂, —Si(R^{B})₃, —Si(R^{B})₂OR^{B}, —Si(R^{B})(OR^{B})₂, —Si(OR^{B})₃, —OSi(R^{B})₃, —OSi(R^{B})₂OR^{B}, —OSi(R^{B})(OR^{B})₂, or —OSi(OR^{B})₃;
each of R¹⁴³, R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl; and
each instance of R^{B} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{B} attached to the same intervening atom are joined together with the intervening atom to form an optionally substituted, monocyclic, heterocyclic or heteroaryl ring; or
the pharmaceutical composition of claim 160 or 161.

193. The method of any one of claims 163-192, wherein the subject is a human.

194. The method of claim 193, wherein the subject is a human aged 18 and older.

195. A method of inhibiting the activity and/or production of a glycogen synthase kinase 3 (GSK3) in a cell, tissue, or biological sample the method comprising contacting the cell, tissue, or biological sample with an effective amount of:
the compound of any one of claims 1-159, or pharmaceutically acceptable salt, solvate, hydrate, polymorph, co-crystal, tautomer, stereoisomer, isotopically labeled compound, or prodrug thereof;
a compound of the formula: 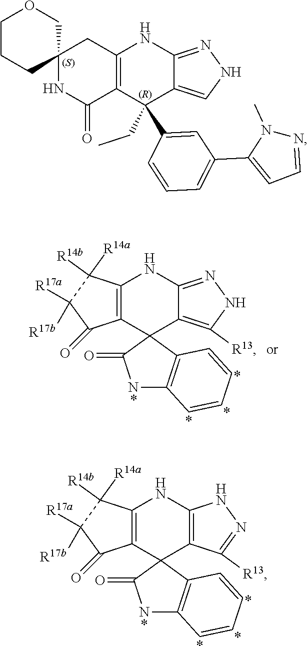
or a pharmaceutically acceptable salt, solvate, hydrate, polymorph, co-crystal, tautomer, each instance of the atoms marked with * is independently optionally substituted; [symbol]
is a single or double bond;
R¹³ is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{B}, —SCN, —SR^{B}, —SSR^{B}, —N₃, —NO, —N(R^{B})₂, —NO₂, —C(═O)R^{B}, —C(═O)OR^{B}, —C(═O)SR^{B}, —C(═O)N(R^{B})₂, —C(═NR^{B})R^{B}, —C(═NR^{B})OR^{B}, —C(═NR^{B})SR^{B}, —C(═NR^{B})N(R^{B})₂, —S(═O)R^{B}, —S(═O)OR^{B}, —S(═O)SR^{B}, —S(═O)N(R^{B}), —S(═O)₂R^{B}, —S(═O)₂OR^{B}, —S(═O)₂SR^{B}, —S(═O)₂N(R^{B})₂, —OC(═O)R^{B}, —OC(═O)OR^{B}, —OC(═O)SR^{B}, —OC(═O)N(R^{B})₂, —OC(═NR^{B})R^{B}, —OC(═NR^{B})OR^{B}, —OC(═NR^{B})SR^{B}, —OC(═NR^{B})N(R^{B})₂, —OS(═O)R^{B}, —OS(═O)OR^{B}, —OS(═O)SR^{B}, —OS(═O)N(R^{B})₂, —OS(═O)₂R^{B}, —OS(═O)₂OR^{B}, —OS(═O)₂SR^{B}, —OS(═O)₂N(R^{B})₂, —ON(R^{B})₂, —SC(═O)R^{B}, —SC(═O)OR^{B}, —SC(═O)SR^{B}, —SC(═O)N(R^{B})₂, —SC(═NR^{B})R^{B}, —SC(═NR^{B})OR^{B}, —SC(═NR^{B})SR^{B}, —SC(═NR^{B})N(R^{B})₂, —NR^{B}C(═O)R^{B}, —NR^{B}C(═O)OR^{B}, —NR^{B}C(═O)SR^{B}, —NR^{B}C(═O)N(R^{B})₂, —NR^{B}C(═NR^{B})R^{B}, —NR^{B}C(═NR^{B})OR^{B}, —NR^{B}C(═NR^{B})SR^{B}, —NR^{B}C(═NR^{B})N(R^{B})₂, —NR^{B}S(═O)R^{B}, —NR^{B}S(═O)OR^{B}, —NR^{B}S(═O)SR^{B}, —NR^{B}S(═O)N(R^{B})₂, —NR^{B}S(═O)₂R^{B}, —NR^{B}S(═O)₂OR^{B}, —NR^{B}S(═O)₂SR^{B}, —NR^{B}S(═O)₂N(R^{B})₂, —Si(R^{B})₃, —Si(R^{B})₂OR^{B}, —Si(R^{B})(OR^{B})₂, —Si(OR^{B})₃, —OSi(R^{B})₃, —OSi(R^{E})₂OR^{B}, —OSi(R^{B})(OR^{B})₂, or —OSi(OR^{B})₃;
each of R^{14a}, R^{14b}, R^{17a}, and R^{17b} is independently hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, or optionally substituted heteroaryl; and
each instance of R^{E} is independently hydrogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two instances of R^{B} attached to the same intervening atom are joined together with the intervening atom to form an optionally substituted, monocyclic, heterocyclic or heteroaryl ring; or
the pharmaceutical composition of claim 160 or 161.

196. The method of claim 195, wherein the cell, tissue, or biological sample is in vitro.

197. The method of any one of claims 163-196, wherein the GSK3 is glycogen synthase kinase 3 α (GSK3α).

198. The method of claim 197, wherein the compound, or pharmaceutically acceptable salt, solvate, hydrate, polymorph, co-crystal, tautomer, stereoisomer, isotopically labeled compound, or prodrug thereof, is at least three times more selective for inhibiting the activity and/or production of GSK3α than glycogen synthase kinase 3 β (GSK3β) in an in vitro assay.

199. The method of any one of claims 163-196, wherein the GSK3 is glycogen synthase kinase 3 β (GSK3β).

200. The method of claim 199, wherein the compound, or pharmaceutically acceptable salt, solvate, hydrate, polymorph, co-crystal, tautomer, stereoisomer, isotopically labeled compound, or prodrug thereof, is at least three times more selective for inhibiting the activity and/or production of GSK3β than glycogen synthase kinase 3 α (GSK3α) in an in vitro assay.
